(19) United States
(12) Patent Application Publication
(10) Pub. No.: US 2026/0242768 A1
(43) Pub. Date: Aug. 20, 2026

(54) ACETYL-COENZYME A CARBOXYLASE MUTANTS

(71) Applicants: CIBUS US LLC, San Diego, CA (US); CIBUS EUROPE B.V., Breda (NL)

(72) Inventors: Gregory F.W. GOCAL, San Diego, CA (US); Peter R. BEETHAM, San Diego, CA (US); Keith A. WALKER, San Diego, CA (US); Steven L. SANDERS, San Diego, CA (US); Dolendro NAMEIRAKPAM, San Diego, CA (US)

(73) Assignees: CIBUS US LLC, San Diego, CA (US); CIBUS EUROPE B.V., Breda (NL)

(21) Appl. No.: 19/471,143

(22) PCT Filed: Mar. 31, 2024
(86) PCT No.: PCT/US2024/022430

§ 371 Date: Sep. 30, 2025

Related U.S. Application Data
(60) Provisional application No. 63/493,442, filed on Mar. 31, 2023.

(51) Int. Cl. C12N 9/00; C12N 15/82

(52) U.S. Cl. CPC C12N 9/93; C12N 15/8274; C12Y 604/01002

(57) ABSTRACT

The present disclosure provides plants and/or plant cells bearing one or more mutations in an acetyl-coenzyme A carboxylase (ACCase) gene, and methods of making and using such plants. In some embodiments, the plants and/or plant cells are resistant or tolerant to an herbicide which targets acetyl-coenzyme A carboxylase.

### CROSS-REFERENCE TO RELATED APPLICATIONS

The present invention claims the benefit of priority to U.S. Provisional Application Ser. No. 63/493,442, filed on Mar. 31, 2023, the contents of which are incorporated herein by reference in its entirety.

### FIELD

The present disclosure relates to compositions and methods pertaining to novel plant genes and gene products, and to plants having one or more gene mutations. In particular, the present disclosure provides acetyl-coenzyme A carboxylase (ACCase) genes and plants and/or plant cells bearing one or more mutations in an ACCase gene, as well as methods of making and using such plants. In some embodiments, the plant has increased resistance or tolerance to ACCase inhibitor herbicides including clethodim.

### BACKGROUND

Acetyl-CoA carboxylase or acetyl-coenzyme A carboxylase (ACCase) is a biotin-dependent enzyme that catalyzes the irreversible carboxylation of acetyl-CoA to produce malonyl-CoA through its two catalytic activities, biotin carboxylase (BC) and carboxyltransferase (CT). ACCase is a multi-subunit enzyme in most prokaryotes and in the chloroplasts of most plants and algae, whereas it is a large, multi-domain enzyme in the endoplasmic reticulum. The most important function of ACCase is to provide the malonyl-CoA substrate for the biosynthesis of fatty acids.

ACCase inhibitors are a broadly used class of herbicides that preferentially kill grasses. ACCase inhibitors specifically target the plastidal form of the ACCase enzyme present in grass species.

PCT application WO/2015/139008 (US2015/020622), hereby incorporated by reference in its entirety, discloses methods to cause targeted changes to a DNA sequence. PCT application WO 2011/028833 (US2013/0111618), hereby incorporated by reference in its entirety, discloses herbicide-tolerant plants and methods of producing and treating herbicide-tolerant plants.

### BRIEF SUMMARY

The present disclosure is based at least in part on the discovery that certain mutations in the ACCase gene in a plant may result in certain advantages or desirable properties; for example, in some embodiments, may result in resistance to herbicides.

Accordingly, provided herein include a plant or plant cell having one or more mutations in the ACCase gene and methods of generating such a plant or plant cell. In some embodiments the plant or plant cells are non-transgenic. In certain embodiments the plant has increased resistance or tolerance to an herbicide, for example an herbicide that is a member of the aryloxyphenoxypropanoate, cyclohexanedione, and/or phenylpyrazoline families of herbicides. In various embodiments such plant or plant cell exhibits substantially normal growth or development of the plant, its organs, tissues or cells, in the presence of an herbicide as compared to the corresponding wild-type plant or cell.

Unless specifically indicated otherwise, the blackgrass (Alopecurus myosuroides) amino acid numbering depicted in SEQ ID NO: 2 is used herein as the reference for plastidal ACCase. Those of ordinary skill in the art are able to identify analogous corresponding amino acid residues in ACCase genes of ACCase homologs, including orthologs (ACCase from different species) and paralogs (ACCase from the same species). An example of such is shown in the below in which the ACCase protein sequences in Alopecurus myosuroides (FIG. 1B and SEQ ID NO: 2), Oryza sativa indica (FIG. 2B and SEQ ID NO: 4) and Oryza sativa japonica (FIG. 3 and SEQ ID NO: 6) are compared, and the listed corresponding amino acid positions identified.

Exemplary Amino Acid Positions

| Am (blackgrass | | |
|---|---|---|
| (Alopecurus | | |
| myosuroides) | Os Indica | Os Japonica |
| L1543 | L1548 | L1548 |
| V1529 | V1534 | V1534 |
| R1672 | R1683 | R1670 |
| D1681 | D1692 | D1679 |
| I1781 | I1792 | I1779 |
| S1784 | S1795 | S1782 |
| A1785 | A1796 | A1783 |
| L1818 | L1829 | L1816 |
| K1840 | K1851 | K1838 |
| M1974 | M1985 | M1972 |
| A1957 | A1968 | A1955 |
| V2075 | V2086 | V2073 |
| D2078 | D2089 | D2076 |
| P2083 | P2094 | P2081 |
| C2088 | C2099 | C2086 |
| N2097 | N2108 | N2095 |
| V2098 | V2109 | V2096 |
| E2112 | E2123 | E2110 |
| I2149 | I2161 | I2148 |
| A2226 | A2238 | A2225 |
| R2257 | M2264 | M2251 |
| N2262 | N2269 | N2256 |

In conjunction with the various aspects, embodiments, compositions, and methods disclosed herein, a plant or plant cell may include a mutated ACCase gene wherein a position of the ACCase gene is changed from the plastidal ACCase amino acid residue to the cytosolic ACCase amino acid residue.

In one aspect provided is a plant or plant cell having a mutated ACCase gene wherein the gene encodes an ACCase protein including one or more substitutions at one or more amino acid positions corresponding to a position or position combinations selected from the group consisting of the mutations listed on Table 1 and/or methods of making such a plant and or plant cell. In one aspect provided is a plant or plant cell having a mutated ACCase gene wherein the gene encodes an ACCase protein comprising one or more substitutions at one or more amino acid positions corresponding to a position or position combinations selected from the group consisting of the mutations listed on Table 2 and/or methods of making such a plant and or plant cell. In one aspect provided is a plant or plant cell having a mutated ACCase gene wherein the gene encodes an ACCase protein comprising one or more substitutions at one or more amino acid positions corresponding to a position or position combinations selected from the group consisting of the mutations listed on Table 3 and/or methods of making such a plant and or plant cell.

In certain embodiments, a plant or plant cell may include a mutated ACCase gene wherein the gene encodes an ACCase protein comprising a substitution at one or more amino acid positions corresponding to a position selected from the group consisting of N1460, V1529, L1543, L1555, C1575, S1605, G1605-6A*, N1607, L1649, R1672, D1681, F1683, V1715, D1725, R1734, M1738, T1739, A1752, Q1756, I1766, K1772, N1780, I1781, G1783, S1784, A1785, A1786, S1792, I1811, L1818, Q1824, A1837, K1840, V1864, P1870, S1922, K1930, K1938, E1946, A1957, V1966, I1967, M1974, M1975, V1992, W1999, A2004, W2027, E2039, I2041, V2049, A2059, A2067, W2074, V2075, D2078, S2079, K2080, I2081, P2083, R2085, C2088, K2095, G2096, N2097, V2098, E2112, D2127, 12149, E2150, K2154, Q2155, L2159, E2169, A2178, A2179, V2182, F2196, H2223, A2226, K2232, A2241, S2244, F2253, V2254, R2257, N2262, K2264, I2267, S2276, S2280, S2297, L2299, K2302, and K2317 of SEQ ID NO. 2.

In certain embodiments, a plant or plant cell may include a mutated ACCase gene wherein the gene encodes an ACCase protein comprising a substitution corresponding to a substitution selected from the group consisting of N1460S, V1529M, L1543F, L1543V, L1555P, C1575S, S1605Y, G1605-6A*, N1607S, L1649M, R1672I, D1681E, D1681G, F1683L, V1715A, D1725E, R1734G, M1738L, T1739S, A1752T, Q1756P, Q1756E, I1766V, K1772E, K1772N, N1780D, I1781L, I1781T, I1781V, I1781A, G1783C, S1784G, S1784N, A1785G, A1786P, S1792L, I1811N, L1818H, L1818I, Q1824P, A1837V, K1840M, K1840R, V1864F, P1870R, S1922G, K1930N, K1938R, E1946G, E1946D, E1946V, A1957T, V1966L, I1967V, M1974T, M1975T, V1992D, W1999C, W1999G, W1999L, W1999S, A2004V, W2027C, W2027R, E2039D, E2039G, I2041D, I2041N, I2041V, V2049F, V2049I, V2049L, V2049T, V2049C, V2049A, V2049S, A2059V, A2067S, W2074L, V2075M, V2075G, V2075I, V2075L, D2078G, D2078K, D2078T, D2078V, S2079F, S2079P, K2080E, P2083N, P2083Q, P2083S, P2083T, R2085S, C2088M, C2088N, C2088R, C2088W, C2088F, C2088G, C2088H, C2088K, C2088S, C2088T, C2088L, C2088V, K2095E, K2095N, G2096A, G2096S, N2097D, N2097S, N2097R, N2097K, N2097E, N2097H, N2097A, N2097G, N2097C, N2097P, N2097L, N2097I, N2097M, N2097F, N2097W, N2097Q, N2097T, N2097Y, V2098A, V2098H, V2098P, V2098S, V2098G, V2098C, E2112G, E2112V, D2127N, I2149K, I2149V, E2150K, A2151T, K2154R, Q2155L, L2159P, E2169D, A2178I, A2179T, V2182A, F2196Y, H2223R, A2226S, K2232N, A2241T, F2253I, V2254I, N2262K, K2264R, I2267V, S2276F, S2276P, S2280P, S2297C, L2299I, K2302Q, and K2317T of SEQ ID NO: 2.

In certain embodiments, a plant or plant cell may include a mutated ACCase gene wherein the gene encodes an ACCase protein comprising a substitution corresponding to a substitution selected from the group consisting of V1529M, L1543F, L1543V, R1672I, D1681E, D1681G, S1784G, S1784N, A1785G, L1818H, L1818I, K1840M, K1840R, A1957T, M1974T, M1975T, D2078G, P2083T, N2097S, V2098G, E2112V, I2149K, A2226S, and N2262K of SEQ ID NO: 2. In certain embodiments, a plant or plant cell may include a mutated ACCase gene wherein the gene encodes an ACCase protein comprising a substitution corresponding to a substitution selected from the group consisting of V1529M, L1543F, L1543V, L1555P, C1575S, S1605Y, G1605-6A*, L1649M, R1672I, D1681E, D1681G, F1683L, V1715A, D1725E, A1752T, I1766V, K1772E, S1784G, S1784N, L1818H, L1818I, K1840M, K1840R, K1930N, A1957T, V1966L, M1974T, M1975T, V1992D, A2067S, R2085S, E2112V, D2127N, I2149K, E2150K, A2151T, K2154R, Q2155L, L2159P, I2267V, E2169D, A2178I, A2179T, V2182A, F2196Y, H2223R, A2226S, K2232N, F2253I, V2254I, N2262K, S2276F, S2276P, S2280P, S2297C, L2299I, K2302Q, and K2317T of SEQ ID NO: 2.

In certain embodiments, a plant or plant cell may include a mutated ACCase gene wherein the gene encodes an ACCase protein comprising a substitution corresponding to N1460S of SEQ ID NO: 2. In certain embodiments, a plant or plant cell may include a mutated ACCase gene wherein the gene encodes an ACCase protein comprising a substitution corresponding to V1529M of SEQ ID NO: 2. In certain embodiments, a plant or plant cell may include a mutated ACCase gene wherein the gene encodes an ACCase protein comprising a substitution corresponding to L1543F of SEQ ID NO: 2. In certain embodiments, a plant or plant cell may include a mutated ACCase gene wherein the gene encodes an ACCase protein comprising a substitution corresponding to L1543V of SEQ ID NO: 2. In certain embodiments, a plant or plant cell may include a mutated ACCase gene wherein the gene encodes an ACCase protein comprising a substitution corresponding to L1555P of SEQ ID NO: 2. In certain embodiments, a plant or plant cell may include a mutated ACCase gene wherein the gene encodes an ACCase protein comprising a substitution corresponding to C1575S of SEQ ID NO: 2. In certain embodiments, a plant or plant cell may include a mutated ACCase gene wherein the gene encodes an ACCase protein comprising a substitution corresponding to S1605Y of SEQ ID NO: 2. In certain embodiments, a plant or plant cell may include a mutated ACCase gene wherein the gene encodes an ACCase protein comprising a substitution corresponding to G1605-6A* as described herein. In certain embodiments, a plant or plant cell may include a mutated ACCase gene wherein the gene encodes an ACCase protein comprising a substitution corresponding to N1607S of SEQ ID NO: 2. In certain embodiments, a plant or plant cell may include a mutated ACCase gene wherein the gene encodes an ACCase protein comprising a substitution corresponding to L1649M of SEQ ID NO: 2. In certain embodiments, a plant or plant cell may include a mutated ACCase gene wherein the gene encodes an ACCase protein comprising a substitution corresponding to R16721 of SEQ ID NO: 2. In certain embodiments, a plant or plant cell may include a mutated ACCase gene wherein the gene encodes an ACCase protein comprising a substitution corresponding to D1681E of SEQ ID NO: 2. In certain embodiments, a plant or plant cell may include a mutated ACCase gene wherein the gene encodes an ACCase protein comprising a substitution corresponding to D1681G of SEQ ID NO: 2. In certain embodiments, a plant or plant cell may include a mutated ACCase gene wherein the gene encodes an ACCase protein comprising a substitution corresponding to F1683L of SEQ ID NO: 2. In certain embodiments, a plant or plant cell may include a mutated ACCase gene wherein the gene encodes an ACCase protein comprising a substitution corresponding to V1715A of SEQ ID NO: 2. In certain embodiments, a plant or plant cell may include a mutated ACCase gene wherein the gene encodes an ACCase protein comprising a substitution corresponding to D1725E of SEQ ID NO: 2. In certain embodiments, a plant or plant cell may include a mutated ACCase gene wherein the gene encodes an ACCase protein comprising a substitution corresponding to R1734G of SEQ ID NO: 2. In certain embodiments, a plant or plant cell may include a mutated ACCase gene wherein the gene encodes an ACCase protein comprising a substitution corresponding to M1738L of SEQ ID NO: 2. In certain embodiments, a plant or plant cell may include a mutated ACCase gene wherein the gene encodes an ACCase protein comprising a substitution corresponding to T1739S of SEQ ID NO: 2. In certain embodiments, a plant or plant cell may include a mutated ACCase gene wherein the gene encodes an ACCase protein comprising a substitution corresponding to A1752T of SEQ ID NO: 2. In certain embodiments, a plant or plant cell may include a mutated ACCase gene wherein the gene encodes an ACCase protein comprising a substitution corresponding to Q1756P of SEQ ID NO: 2. In certain embodiments, a plant or plant cell may include a mutated ACCase gene wherein the gene encodes an ACCase protein comprising a substitution corresponding to Q1756E of SEQ ID NO: 2. In certain embodiments, a plant or plant cell may include a mutated ACCase gene wherein the gene encodes an ACCase protein comprising a substitution corresponding to I1766V of SEQ ID NO: 2. In certain embodiments, a plant or plant cell may include a mutated ACCase gene wherein the gene encodes an ACCase protein comprising a substitution corresponding to K1772E of SEQ ID NO: 2. In certain embodiments, a plant or plant cell may include a mutated ACCase gene wherein the gene encodes an ACCase protein comprising a substitution corresponding to K1772N of SEQ ID NO: 2. In certain embodiments, a plant or plant cell may include a mutated ACCase gene wherein the gene encodes an ACCase protein comprising a substitution corresponding to N1780D of SEQ ID NO: 2. In certain embodiments, a plant or plant cell may include a mutated ACCase gene wherein the gene encodes an ACCase protein comprising a substitution corresponding to I1781L of SEQ ID NO: 2. In certain embodiments, a plant or plant cell may include a mutated ACCase gene wherein the gene encodes an ACCase protein comprising a substitution corresponding to I1781T of SEQ ID NO: 2. In certain embodiments, a plant or plant cell may include a mutated ACCase gene wherein the gene encodes an ACCase protein comprising a substitution corresponding to I1781V of SEQ ID NO: 2. In certain embodiments, a plant or plant cell may include a mutated ACCase gene wherein the gene encodes an ACCase protein comprising a substitution corresponding to I1781A of SEQ ID NO: 2. In certain embodiments, a plant or plant cell may include a mutated ACCase gene wherein the gene encodes an ACCase protein comprising a substitution corresponding to G1783C of SEQ ID NO: 2. In certain embodiments, a plant or plant cell may include a mutated ACCase gene wherein the gene encodes an ACCase protein comprising a substitution corresponding to S1784G of SEQ ID NO: 2. In certain embodiments, a plant or plant cell may include a mutated ACCase gene wherein the gene encodes an ACCase protein comprising a substitution corresponding to S1784N of SEQ ID NO: 2. In certain embodiments, a plant or plant cell may include a mutated ACCase gene wherein the gene encodes an ACCase protein comprising a substitution corresponding to A1785G of SEQ ID NO: 2. In certain embodiments, a plant or plant cell may include a mutated ACCase gene wherein the gene encodes an ACCase protein comprising a substitution corresponding to A1786P of SEQ ID NO: 2. In certain embodiments, a plant or plant cell may include a mutated ACCase gene wherein the gene encodes an ACCase protein comprising a substitution corresponding to S1792L of SEQ ID NO: 2. In certain embodiments, a plant or plant cell may include a mutated ACCase gene wherein the gene encodes an ACCase protein comprising a substitution corresponding to I1811N of SEQ ID NO: 2. In certain embodiments, a plant or plant cell may include a mutated ACCase gene wherein the gene encodes an ACCase protein comprising a substitution corresponding to L1818H of SEQ ID NO: 2. In certain embodiments, a plant or plant cell may include a mutated ACCase gene wherein the gene encodes an ACCase protein comprising a substitution corresponding to L1818I of SEQ ID NO: 2. In certain embodiments, a plant or plant cell may include a mutated ACCase gene wherein the gene encodes an ACCase protein comprising a substitution corresponding to Q1824P of SEQ ID NO: 2. In certain embodiments, a plant or plant cell may include a mutated ACCase gene wherein the gene encodes an ACCase protein comprising a substitution corresponding to A1837V of SEQ ID NO: 2. In certain embodiments, a plant or plant cell may include a mutated ACCase gene wherein the gene encodes an ACCase protein comprising a substitution corresponding to K1840M of SEQ ID NO: 2. In certain embodiments, a plant or plant cell may include a mutated ACCase gene wherein the gene encodes an ACCase protein comprising a substitution corresponding to K1840R of SEQ ID NO: 2. In certain embodiments, a plant or plant cell may include a mutated ACCase gene wherein the gene encodes an ACCase protein comprising a substitution corresponding to V1864F of SEQ ID NO: 2. In certain embodiments, a plant or plant cell may include a mutated ACCase gene wherein the gene encodes an ACCase protein comprising a substitution corresponding to P1870R of SEQ ID NO: 2. In certain embodiments, a plant or plant cell may include a mutated ACCase gene wherein the gene encodes an ACCase protein comprising a substitution corresponding to S1922G of SEQ ID NO: 2. In certain embodiments, a plant or plant cell may include a mutated ACCase gene wherein the gene encodes an ACCase protein comprising a substitution corresponding to K1930N, of SEQ ID NO: 2. In certain embodiments, a plant or plant cell may include a mutated ACCase gene wherein the gene encodes an ACCase protein comprising a substitution corresponding to K1938R of SEQ ID NO: 2. In certain embodiments, a plant or plant cell may include a mutated ACCase gene wherein the gene encodes an ACCase protein comprising a substitution corresponding to E1946V, of SEQ ID NO: 2. In certain embodiments, a plant or plant cell may include a mutated ACCase gene wherein the gene encodes an ACCase protein comprising a substitution corresponding to E1946G, of SEQ ID NO: 2. In certain embodiments, a plant or plant cell may include a mutated ACCase gene wherein the gene encodes an ACCase protein comprising a substitution corresponding to E1946D, of SEQ ID NO: 2. In certain embodiments, a plant or plant cell may include a mutated ACCase gene wherein the gene encodes an ACCase protein comprising a substitution corresponding to A1957T of SEQ ID NO: 2. In certain embodiments, a plant or plant cell may include a mutated ACCase gene wherein the gene encodes an ACCase protein comprising a substitution corresponding to V1966L of SEQ ID NO: 2. In certain embodiments, a plant or plant cell may include a mutated ACCase gene wherein the gene encodes an ACCase protein comprising a substitution corresponding to 11967V of SEQ ID NO: 2. In certain embodiments, a plant or plant cell may include a mutated ACCase gene wherein the gene encodes an ACCase protein comprising a substitution corresponding to M1974T of SEQ ID NO: 2. In certain embodiments, a plant or plant cell may include a mutated ACCase gene wherein the gene encodes an ACCase protein comprising a substitution corresponding to M1975T of SEQ ID NO: 2. In certain embodiments, a plant or plant cell may include a mutated ACCase gene wherein the gene encodes an ACCase protein comprising a substitution corresponding to V1992D of SEQ ID NO: 2. In certain embodiments, a plant or plant cell may include a mutated ACCase gene wherein the gene encodes an ACCase protein comprising a substitution corresponding to W1999C, of SEQ ID NO: 2. In certain embodiments, a plant or plant cell may include a mutated ACCase gene wherein the gene encodes an ACCase protein comprising a substitution corresponding to W1999G, of SEQ ID NO: 2. In certain embodiments, a plant or plant cell may include a mutated ACCase gene wherein the gene encodes an ACCase protein comprising a substitution corresponding to W1999L, of SEQ ID NO: 2. In certain embodiments, a plant or plant cell may include a mutated ACCase gene wherein the gene encodes an ACCase protein comprising a substitution corresponding to W1999S, of SEQ ID NO: 2. In certain embodiments, a plant or plant cell may include a mutated ACCase gene wherein the gene encodes an ACCase protein comprising a substitution corresponding to A2004V, of SEQ ID NO: 2. In certain embodiments, a plant or plant cell may include a mutated ACCase gene wherein the gene encodes an ACCase protein comprising a substitution corresponding to W2027C, of SEQ ID NO: 2. In certain embodiments, a plant or plant cell may include a mutated ACCase gene wherein the gene encodes an ACCase protein comprising a substitution corresponding to W2027R, of SEQ ID NO: 2. In certain embodiments, a plant or plant cell may include a mutated ACCase gene wherein the gene encodes an ACCase protein comprising a substitution corresponding to E2039D, of SEQ ID NO: 2. In certain embodiments, a plant or plant cell may include a mutated ACCase gene wherein the gene encodes an ACCase protein comprising a substitution corresponding to E2039G, of SEQ ID NO: 2. In certain embodiments, a plant or plant cell may include a mutated ACCase gene wherein the gene encodes an ACCase protein comprising a substitution corresponding to 12041D, of SEQ ID NO: 2. In certain embodiments, a plant or plant cell may include a mutated ACCase gene wherein the gene encodes an ACCase protein comprising a substitution corresponding to I2041N, of SEQ ID NO: 2. In certain embodiments, a plant or plant cell may include a mutated ACCase gene wherein the gene encodes an ACCase protein comprising a substitution corresponding to I2041V, of SEQ ID NO: 2. In certain embodiments, a plant or plant cell may include a mutated ACCase gene wherein the gene encodes an ACCase protein comprising a substitution corresponding to V2049I of SEQ ID NO: 2. In certain embodiments, a plant or plant cell may include a mutated ACCase gene wherein the gene encodes an ACCase protein comprising a substitution corresponding to V2049F of SEQ ID NO: 2. In certain embodiments, a plant or plant cell may include a mutated ACCase gene wherein the gene encodes an ACCase protein comprising a substitution corresponding to V2049L of SEQ ID NO: 2. In certain embodiments, a plant or plant cell may include a mutated ACCase gene wherein the gene encodes an ACCase protein comprising a substitution corresponding to V2049T of SEQ ID NO: 2. In certain embodiments, a plant or plant cell may include a mutated ACCase gene wherein the gene encodes an ACCase protein comprising a substitution corresponding to V2049C of SEQ ID NO: 2. In certain embodiments, a plant or plant cell may include a mutated ACCase gene wherein the gene encodes an ACCase protein comprising a substitution corresponding to V2049A of SEQ ID NO: 2. In certain embodiments, a plant or plant cell may include a mutated ACCase gene wherein the gene encodes an ACCase protein comprising a substitution corresponding to V2049S of SEQ ID NO: 2. In certain embodiments, a plant or plant cell may include a mutated ACCase gene wherein the gene encodes an ACCase protein comprising a substitution corresponding to A2059V of SEQ ID NO: 2. In certain embodiments, a plant or plant cell may include a mutated ACCase gene wherein the gene encodes an ACCase protein comprising a substitution corresponding to A2067S of SEQ ID NO: 2. In certain embodiments, a plant or plant cell may include a mutated ACCase gene wherein the gene encodes an ACCase protein comprising a substitution corresponding to W2074L of SEQ ID NO: 2. In certain embodiments, a plant or plant cell may include a mutated ACCase gene wherein the gene encodes an ACCase protein comprising a substitution corresponding to V2075I of SEQ ID NO: 2. In certain embodiments, a plant or plant cell may include a mutated ACCase gene wherein the gene encodes an ACCase protein comprising a substitution corresponding to V2075L of SEQ ID NO: 2. In certain embodiments, a plant or plant cell may include a mutated ACCase gene wherein the gene encodes an ACCase protein comprising a substitution corresponding to V2075M of SEQ ID NO: 2. In certain embodiments, a plant or plant cell may include a mutated ACCase gene wherein the gene encodes an ACCase protein comprising a substitution corresponding to V2075G of SEQ ID NO: 2. In certain embodiments, a plant or plant cell may include a mutated ACCase gene wherein the gene encodes an ACCase protein comprising a substitution corresponding to D2078G of SEQ ID NO: 2. In certain embodiments, a plant or plant cell may include a mutated ACCase gene wherein the gene encodes an ACCase protein comprising a substitution corresponding to D2078K of SEQ ID NO: 2. In certain embodiments, a plant or plant cell may include a mutated ACCase gene wherein the gene encodes an ACCase protein comprising a substitution corresponding to D2078T of SEQ ID NO: 2. In certain embodiments, a plant or plant cell may include a mutated ACCase gene wherein the gene encodes an ACCase protein comprising a substitution corresponding to D2078V of SEQ ID NO: 2. In certain embodiments, a plant or plant cell may include a mutated ACCase gene wherein the gene encodes an ACCase protein comprising a substitution corresponding to S2079F of SEQ ID NO: 2. In certain embodiments, a plant or plant cell may include a mutated ACCase gene wherein the gene encodes an ACCase protein comprising a substitution corresponding to S2079P of SEQ ID NO: 2. In certain embodiments, a plant or plant cell may include a mutated ACCase gene wherein the gene encodes an ACCase protein comprising a substitution corresponding to K2080E of SEQ ID NO: 2. In certain embodiments, a plant or plant cell may include a mutated ACCase gene wherein the gene encodes an ACCase protein comprising a substitution corresponding to P2083N of SEQ ID NO: 2. In certain embodiments, a plant or plant cell may include a mutated ACCase gene wherein the gene encodes an ACCase protein comprising a substitution corresponding to P2083Q of SEQ ID NO: 2. In certain embodiments, a plant or plant cell may include a mutated ACCase gene wherein the gene encodes an ACCase protein comprising a substitution corresponding to P2083S of SEQ ID NO: 2. In certain embodiments, a plant or plant cell may include a mutated ACCase gene wherein the gene encodes an ACCase protein comprising a substitution corresponding to P2083T of SEQ ID NO: 2. In certain embodiments, a plant or plant cell may include a mutated ACCase gene wherein the gene encodes an ACCase protein comprising a substitution corresponding to R2085S of SEQ ID NO: 2. In certain embodiments, a plant or plant cell may include a mutated ACCase gene wherein the gene encodes an ACCase protein comprising a substitution corresponding to C2088M of SEQ ID NO: 2. In certain embodiments, a plant or plant cell may include a mutated ACCase gene wherein the gene encodes an ACCase protein comprising a substitution corresponding to C2088N of SEQ ID NO: 2. In certain embodiments, a plant or plant cell may include a mutated ACCase gene wherein the gene encodes an ACCase protein comprising a substitution corresponding to C2088R of SEQ ID NO: 2. In certain embodiments, a plant or plant cell may include a mutated ACCase gene wherein the gene encodes an ACCase protein comprising a substitution corresponding to C2088W of SEQ ID NO: 2. In certain embodiments, a plant or plant cell may include a mutated ACCase gene wherein the gene encodes an ACCase protein comprising a substitution corresponding to C2088F of SEQ ID NO: 2. In certain embodiments, a plant or plant cell may include a mutated ACCase gene wherein the gene encodes an ACCase protein comprising a substitution corresponding to C2088G of SEQ ID NO: 2. In certain embodiments, a plant or plant cell may include a mutated ACCase gene wherein the gene encodes an ACCase protein comprising a substitution corresponding to C2088H of SEQ ID NO: 2. In certain embodiments, a plant or plant cell may include a mutated ACCase gene wherein the gene encodes an ACCase protein comprising a substitution corresponding to C2088K of SEQ ID NO: 2. In certain embodiments, a plant or plant cell may include a mutated ACCase gene wherein the gene encodes an ACCase protein comprising a substitution corresponding to C2088S of SEQ ID NO: 2. In certain embodiments, a plant or plant cell may include a mutated ACCase gene wherein the gene encodes an ACCase protein comprising a substitution corresponding to C2088T of SEQ ID NO: 2. In certain embodiments, a plant or plant cell may include a mutated ACCase gene wherein the gene encodes an ACCase protein comprising a substitution corresponding to C2088L of SEQ ID NO: 2. In certain embodiments, a plant or plant cell may include a mutated ACCase gene wherein the gene encodes an ACCase protein comprising a substitution corresponding to C2088V of SEQ ID NO: 2. In certain embodiments, a plant or plant cell may include a mutated ACCase gene wherein the gene encodes an ACCase protein comprising a substitution corresponding to K2095E of SEQ ID NO: 2. In certain embodiments, a plant or plant cell may include a mutated ACCase gene wherein the gene encodes an ACCase protein comprising a substitution corresponding to K2095N of SEQ ID NO: 2. In certain embodiments, a plant or plant cell may include a mutated ACCase gene wherein the gene encodes an ACCase protein comprising a substitution corresponding to G2096A of SEQ ID NO: 2. In certain embodiments, a plant or plant cell may include a mutated ACCase gene wherein the gene encodes an ACCase protein comprising a substitution corresponding to G2096S of SEQ ID NO: 2. In certain embodiments, a plant or plant cell may include a mutated ACCase gene wherein the gene encodes an ACCase protein comprising a substitution corresponding to N2097D of SEQ ID NO: 2. In certain embodiments, a plant or plant cell may include a mutated ACCase gene wherein the gene encodes an ACCase protein comprising a substitution corresponding to N2097S, of SEQ ID NO: 2. In certain embodiments, a plant or plant cell may include a mutated ACCase gene wherein the gene encodes an ACCase protein comprising a substitution corresponding to N2097R, of SEQ ID NO: 2. In certain embodiments, a plant or plant cell may include a mutated ACCase gene wherein the gene encodes an ACCase protein comprising a substitution corresponding to N2097K, of SEQ ID NO: 2. In certain embodiments, a plant or plant cell may include a mutated ACCase gene wherein the gene encodes an ACCase protein comprising a substitution corresponding to N2097E, of SEQ ID NO: 2. In certain embodiments, a plant or plant cell may include a mutated ACCase gene wherein the gene encodes an ACCase protein comprising a substitution corresponding to N2097H, of SEQ ID NO: 2. In certain embodiments, a plant or plant cell may include a mutated ACCase gene wherein the gene encodes an ACCase protein comprising a substitution corresponding to N2097A, of SEQ ID NO: 2. In certain embodiments, a plant or plant cell may include a mutated ACCase gene wherein the gene encodes an ACCase protein comprising a substitution corresponding to N2097G, of SEQ ID NO: 2. In certain embodiments, a plant or plant cell may include a mutated ACCase gene wherein the gene encodes an ACCase protein comprising a substitution corresponding to N2097C, of SEQ ID NO: 2. In certain embodiments, a plant or plant cell may include a mutated ACCase gene wherein the gene encodes an ACCase protein comprising a substitution corresponding to N2097P, of SEQ ID NO: 2. In certain embodiments, a plant or plant cell may include a mutated ACCase gene wherein the gene encodes an ACCase protein comprising a substitution corresponding to N2097L, of SEQ ID NO: 2. In certain embodiments, a plant or plant cell may include a mutated ACCase gene wherein the gene encodes an ACCase protein comprising a substitution corresponding to N2097I, of SEQ ID NO: 2. In certain embodiments, a plant or plant cell may include a mutated ACCase gene wherein the gene encodes an ACCase protein comprising a substitution corresponding to N2097M, of SEQ ID NO: 2. In certain embodiments, a plant or plant cell may include a mutated ACCase gene wherein the gene encodes an ACCase protein comprising a substitution corresponding to N2097F, of SEQ ID NO: 2. In certain embodiments, a plant or plant cell may include a mutated ACCase gene wherein the gene encodes an ACCase protein comprising a substitution corresponding to N2097W, of SEQ ID NO: 2. In certain embodiments, a plant or plant cell may include a mutated ACCase gene wherein the gene encodes an ACCase protein comprising a substitution corresponding to N2097Q, of SEQ ID NO: 2. In certain embodiments, a plant or plant cell may include a mutated ACCase gene wherein the gene encodes an ACCase protein comprising a substitution corresponding to N2097T, of SEQ ID NO: 2. In certain embodiments, a plant or plant cell may include a mutated ACCase gene wherein the gene encodes an ACCase protein comprising a substitution corresponding to N2097Y, of SEQ ID NO: 2. In certain embodiments, a plant or plant cell may include a mutated ACCase gene wherein the gene encodes an ACCase protein comprising a substitution corresponding to V2098A of SEQ ID NO: 2. In certain embodiments, a plant or plant cell may include a mutated ACCase gene wherein the gene encodes an ACCase protein comprising a substitution corresponding to V2098G, of SEQ ID NO: 2. In certain embodiments, a plant or plant cell may include a mutated ACCase gene wherein the gene encodes an ACCase protein comprising a substitution corresponding to V2098H, of SEQ ID NO: 2. In certain embodiments, a plant or plant cell may include a mutated ACCase gene wherein the gene encodes an ACCase protein comprising a substitution corresponding to V2098P, of SEQ ID NO: 2. In certain embodiments, a plant or plant cell may include a mutated ACCase gene wherein the gene encodes an ACCase protein comprising a substitution corresponding to V2098S, of SEQ ID NO: 2. In certain embodiments, a plant or plant cell may include a mutated ACCase gene wherein the gene encodes an ACCase protein comprising a substitution corresponding to V2098C, of SEQ ID NO: 2. In certain embodiments, a plant or plant cell may include a mutated ACCase gene wherein the gene encodes an ACCase protein comprising a substitution corresponding to E2112G, of SEQ ID NO: 2. In certain embodiments, a plant or plant cell may include a mutated ACCase gene wherein the gene encodes an ACCase protein comprising a substitution corresponding to E2112V of SEQ ID NO: 2. In certain embodiments, a plant or plant cell may include a mutated ACCase gene wherein the gene encodes an ACCase protein comprising a substitution corresponding to D2127N of SEQ ID NO: 2. In certain embodiments, a plant or plant cell may include a mutated ACCase gene wherein the gene encodes an ACCase protein comprising a substitution corresponding to 12149K of SEQ ID NO: 2. In certain embodiments, a plant or plant cell may include a mutated ACCase gene wherein the gene encodes an ACCase protein comprising a substitution corresponding to I2149V, of SEQ ID NO: 2. In certain embodiments, a plant or plant cell may include a mutated ACCase gene wherein the gene encodes an ACCase protein comprising a substitution corresponding to E2150K of SEQ ID NO: 2. In certain embodiments, a plant or plant cell may include a mutated ACCase gene wherein the gene encodes an ACCase protein comprising a substitution corresponding to A2151T of SEQ ID NO: 2. In certain embodiments, a plant or plant cell may include a mutated ACCase gene wherein the gene encodes an ACCase protein comprising a substitution corresponding to K2154R of SEQ ID NO: 2. In certain embodiments, a plant or plant cell may include a mutated ACCase gene wherein the gene encodes an ACCase protein comprising a substitution corresponding to Q2155L of SEQ ID NO: 2. In certain embodiments, a plant or plant cell may include a mutated ACCase gene wherein the gene encodes an ACCase protein comprising a substitution corresponding to L2159P of SEQ ID NO: 2. In certain embodiments, a plant or plant cell may include a mutated ACCase gene wherein the gene encodes an ACCase protein comprising a substitution corresponding to E2169D of SEQ ID NO: 2. In certain embodiments, a plant or plant cell may include a mutated ACCase gene wherein the gene encodes an ACCase protein comprising a substitution corresponding to A21781 of SEQ ID NO: 2. In certain embodiments, a plant or plant cell may include a mutated ACCase gene wherein the gene encodes an ACCase protein comprising a substitution corresponding to A2179T of SEQ ID NO: 2. In certain embodiments, a plant or plant cell may include a mutated ACCase gene wherein the gene encodes an ACCase protein comprising a substitution corresponding to V2182A of SEQ ID NO: 2. In certain embodiments, a plant or plant cell may include a mutated ACCase gene wherein the gene encodes an ACCase protein comprising a substitution corresponding to F2196Y of SEQ ID NO: 2. In certain embodiments, a plant or plant cell may include a mutated ACCase gene wherein the gene encodes an ACCase protein comprising a substitution corresponding to H2223R of SEQ ID NO: 2. In certain embodiments, a plant or plant cell may include a mutated ACCase gene wherein the gene encodes an ACCase protein comprising a substitution corresponding to A2226S of SEQ ID NO: 2. In certain embodiments, a plant or plant cell may include a mutated ACCase gene wherein the gene encodes an ACCase protein comprising a substitution corresponding to K2232N of SEQ ID NO: 2. In certain embodiments, a plant or plant cell may include a mutated ACCase gene wherein the gene encodes an ACCase protein comprising a substitution corresponding to A2241T, of SEQ ID NO: 2. In certain embodiments, a plant or plant cell may include a mutated ACCase gene wherein the gene encodes an ACCase protein comprising a substitution corresponding to F22531 of SEQ ID NO: 2. In certain embodiments, a plant or plant cell may include a mutated ACCase gene wherein the gene encodes an ACCase protein comprising a substitution corresponding to V2254I of SEQ ID NO: 2. In certain embodiments, a plant or plant cell may include a mutated ACCase gene wherein the gene encodes an ACCase protein comprising a substitution corresponding to N2262K of SEQ ID NO: 2. In certain embodiments, a plant or plant cell may include a mutated ACCase gene wherein the gene encodes an ACCase protein comprising a substitution corresponding to K2264R, of SEQ ID NO: 2. In certain embodiments, a plant or plant cell may include a mutated ACCase gene wherein the gene encodes an ACCase protein comprising a substitution corresponding to I2267V of SEQ ID NO: 2. In certain embodiments, a plant or plant cell may include a mutated ACCase gene wherein the gene encodes an ACCase protein comprising a substitution corresponding to S2276F of SEQ ID NO: 2. In certain embodiments, a plant or plant cell may include a mutated ACCase gene wherein the gene encodes an ACCase protein comprising a substitution corresponding to S2276P of SEQ ID NO: 2. In certain embodiments, a plant or plant cell may include a mutated ACCase gene wherein the gene encodes an ACCase protein comprising a substitution corresponding to S2280P of SEQ ID NO: 2. In certain embodiments, a plant or plant cell may include a mutated ACCase gene wherein the gene encodes an ACCase protein comprising a substitution corresponding to S2297C of SEQ ID NO: 2. In certain embodiments, a plant or plant cell may include a mutated ACCase gene wherein the gene encodes an ACCase protein comprising a substitution corresponding to L22991 of SEQ ID NO: 2. In certain embodiments, a plant or plant cell may include a mutated ACCase gene wherein the gene encodes an ACCase protein comprising a substitution corresponding to K2302Q of SEQ ID NO: 2. In certain embodiments, a plant or plant cell may include a mutated ACCase gene wherein the gene encodes an ACCase protein comprising a substitution corresponding to K2317T of SEQ ID NO: 2.

In certain embodiments, a plant or plant cell may include a mutated ACCase gene wherein the gene encodes an ACCase protein comprising a substitution corresponding to N1460S of SEQ ID NO: 2 and an additional substitution or substitution combination such as provided herein (for example a mutation or mutation combination provided in Table 1, Table 2, or Table 3). In certain embodiments, a plant or plant cell may include a mutated ACCase gene wherein the gene encodes an ACCase protein comprising a substitution corresponding to V1529M of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to L1543F of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to L1543V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to L1555P of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C1575S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S1605Y of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to G1605-6A* of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N1607S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to L1649M of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to R16721 of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to D1681E of SEQ ID NO: 2. In certain embodiments, a plant or plant cell may include a mutated ACCase gene wherein the gene encodes an ACCase protein comprising a substitution corresponding to D1681G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to F1683L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V1715A of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to D1725E of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to R1734G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to M1738L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to T1739S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A1752T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to Q1756P of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to Q1756E of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to I1766V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K1772E of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K1772N of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N1780D of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to I1781L of SEQ ID NO: 2. In certain embodiments, a plant or plant cell may include a mutated ACCase gene wherein the gene encodes an ACCase protein comprising a substitution corresponding to I1781T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to I1781V of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to I1781A of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to G1783C of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S1784G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S1784N of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A1785G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A1786P of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S1792L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to I1811N of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to L1818H of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to L1818I of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to Q1824P of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A1837V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K1840M of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K1840R of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V1864F of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to P1870R of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to S1922G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K1930N, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K1938R of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E1946G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E1946D of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E1946V, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A1957T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V1966L of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to I1967V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to M1974T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to M1975T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V1992D of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to W1999C of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to W1999G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to W1999L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to W1999S of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to A2004V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to W2027C of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to W2027R of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E2039D of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E2039G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to I2041D of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to 12041N of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to I2041V of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to V2049F of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2049I of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2049L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2049T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2049C of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2049A of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2049S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A2059V of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to A2067S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A2074L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2075G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2075M of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2075I of SEQ ID NO: 2 and an additional substitution or substitution combination such as provided herein (for example a substitution or substitution combination provided in Table 1, Table 2 or Table 3). In certain embodiments, a plant or plant cell may include a mutated ACCase gene wherein the gene encodes an ACCase protein comprising a substitution corresponding to V2075L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to D2078G of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to D2078K of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to D2078T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to D2078V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S2079F of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S2079P of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K2080E of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to P2083N of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to P2083Q of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to P2083S of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to P2083T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to R2085S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088M of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088N of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088R of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088W of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088F of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088G of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to C2088H of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088K of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to C2088V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K2095E of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K2095N of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to G2096A of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to G2096S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097D of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097S, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097R, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097K, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097E, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097H, of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to N2097A, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097G, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097C, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097P, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097L, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097I, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097M, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097F, of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to N2097W, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097Q, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097T, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2097Y, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2098A of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2098G, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2098H, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2098P, of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to V2098S, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2098C, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E2112G, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E2112V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to D2127N of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to I2149K of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E2150K of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A2151T of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to K2154R of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to Q2155L of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to L2159P of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to E2169D of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A21781 of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A2179T of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2182A of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to F2196Y of SEQ ID NO: 2 and an additional substitution or substitution combination such as provided herein (for example a certain embodiments, a plant or plant cell may include a mutated ACCase gene wherein the gene encodes an ACCase protein comprising a substitution corresponding to H2223R of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to A2226S of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K2232N of SEQ ID NO: 2. In certain embodiments, a plant or plant cell may include a mutated ACCase gene wherein the gene encodes an ACCase protein comprising a substitution corresponding to A2241T, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to F2253I of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to V2254I of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to N2262K of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K2264R, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to I2267V of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S2276F, of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S2276P of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S2280P of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to S2297C of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to L22991 of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K2302Q of SEQ ID NO: 2 and an additional substitution or substitution combination such as provided herein (for example a substitution or substitution combination provided in Table 1, Table 2, or Table 3). In certain embodiments, a plant or plant cell may include a mutated ACCase gene wherein the gene encodes an ACCase protein comprising a substitution corresponding to K2317T of SEQ ID NO: 2 and an additional substitution or substitution combination such as provided herein (for example a substitution or substitution combination provided in Table 1, Table 2, or Table 3).

In certain embodiments, a plant or plant cell provided herein may include a mutated ACCase gene wherein the gene encodes an ACCase protein including a mutation at an amino acid position corresponding to D2078 of SEQ ID NO: 2 (i.e., wherein the encoded protein has any amino acid other than an aspartate at the position corresponding to D2078 of SEQ ID NO: 2). In certain embodiments, a plant or plant cell may include a mutated ACCase gene wherein the gene encodes an ACCase protein comprising a substitution corresponding to D2078G of SEQ ID NO: 2. In certain embodiments, a plant or plant cell may include a mutated ACCase gene wherein the gene encodes an ACCase protein comprising a substitution corresponding to D2078K of SEQ ID NO: 2. In certain embodiments, a plant or plant cell may include a mutated ACCase gene wherein the gene encodes an ACCase protein comprising a substitution corresponding to D2078T of SEQ ID NO: 2. In certain embodiments, a plant or plant cell may include a mutated ACCase gene wherein the gene encodes an ACCase protein comprising a substitution corresponding to D2078V of SEQ ID NO: 2. In some embodiments, a plant or plant cell provided herein may include a mutated ACCase gene wherein the gene encodes an ACCase protein including a mutation at an amino acid position corresponding to D2078 of SEQ ID NO: 2 (such as a substitution as provided herein) in combination with an additional ACCase mutation or mutations as provided herein. In some embodiments, a plant or plant cell provided herein may include a mutated ACCase gene wherein the gene encodes an ACCase protein including a mutation at an amino acid position corresponding to D2078 of SEQ ID NO: 2 (such as a substitution as provided herein) in combination with an additional ACCase mutation or mutations as provided in Table 1. In some embodiments, a plant or plant cell provided herein may include a mutated ACCase gene wherein the gene encodes an ACCase protein including a mutation at an amino acid position corresponding to D2078 of SEQ ID NO: 2 (such as a substitution as provided herein) in combination with an additional ACCase mutation or mutations as provided in Table 2. In some embodiments, a plant or plant cell provided herein may include a mutated ACCase gene wherein the gene encodes an ACCase protein including a mutation at an amino acid position corresponding to D2078 of SEQ ID NO: 2 (such as a substitution as provided herein) in combination with an additional ACCase mutation as provided in Table 3. In some embodiments, a plant or plant cell provided herein may include a mutated ACCase gene wherein the gene encodes an ACCase protein including a mutation at an amino acid position corresponding to D2078 of SEQ ID NO: 2 (such as a substitution as provided herein) in combination with an additional ACCase mutation at an amino acid position corresponding to P2083 of SEQ ID NO: 2.

In certain embodiments, a plant or plant cell provided herein may include a mutated ACCase gene wherein the gene encodes an ACCase protein including a mutation at an amino acid position corresponding to P2083 of SEQ ID NO: 2 (i.e., wherein the encoded protein has any amino acid other than a proline at the position corresponding to P2083 of SEQ ID NO: 2). In certain embodiments, a plant or plant cell may include a mutated ACCase gene wherein the gene encodes an ACCase protein comprising a substitution corresponding to P2083N of SEQ ID NO: 2. In certain embodiments, a plant or plant cell may include a mutated ACCase gene wherein the gene encodes an ACCase protein comprising a substitution corresponding to P2083Q of SEQ ID NO: 2. In certain embodiments, a plant or plant cell may include a mutated ACCase gene wherein the gene encodes an ACCase protein comprising a substitution corresponding to P2083S of SEQ ID NO: 2. In certain embodiments, a plant or plant cell may include a mutated ACCase gene wherein the gene encodes an ACCase protein comprising a substitution corresponding to P2083T of SEQ ID NO: 2. In some embodiments, a plant or plant cell provided herein may include a mutated ACCase gene wherein the gene encodes an ACCase protein including a mutation at an amino acid position corresponding to P2083 of SEQ ID NO: 2 (such as a substitution as provided herein) in combination with an additional ACCase mutation or mutations as provided herein. In some embodiments, a plant or plant cell provided herein may include a mutated ACCase gene wherein the gene encodes an ACCase protein including a mutation at an amino acid position corresponding to P2083 of SEQ ID NO: 2 (such as a substitution as provided herein) in combination with an additional ACCase mutation or mutations as provided in Table 1. In some embodiments, a plant or plant cell provided herein may include a mutated ACCase gene wherein the gene encodes an ACCase protein including a mutation at an amino acid position corresponding to P2083 of SEQ ID NO: 2 (such as a substitution as provided herein) in combination with an additional ACCase mutation or mutations as provided in Table 2. In some embodiments, a plant or plant cell provided herein may include a mutated ACCase gene wherein the gene encodes an ACCase protein including a mutation at an amino acid position corresponding to P2083 of SEQ ID NO: 2 (such as a substitution as provided herein) in combination with an additional ACCase mutation as provided in Table 3. In some embodiments, a plant or plant cell provided herein may include a mutated ACCase gene wherein the gene encodes an ACCase protein including a mutation at an amino acid position corresponding to P2083 of SEQ ID NO: 2 (such as a substitution as provided herein) in combination with an additional ACCase mutation at an amino acid position corresponding to D2078 of SEQ ID NO: 2.

In some embodiments, a plant or plant cell provided herein may have two or more mutations in an ACCase gene such that the mutated gene encodes a protein comprising at least two amino acid substitutions at the amino acid positions corresponding to D2078 and P2083 of SEQ ID NO: 2. In some embodiments, the substitution at the amino acid position corresponding to position D2078 of SEQ ID NO: 2 is selected from the group including, but not limited to: D2078G, D2078K, D2078T, and D2078V. In some embodiments, the substitution at the amino acid position corresponding to position D2078 of SEQ ID NO: 2 is D2078G. In some embodiments, the substitution at the amino acid position corresponding to position P2083 of SEQ ID NO: 2 is selected from the group including, but not limited to: P2083T, P2083S, P2083N, and P2083Q. In some embodiments, the substitution at the amino acid position corresponding to position P2083 of SEQ ID NO: 2 is P2083T. In some embodiments, a plant or plant cell provided herein may have two or more mutations in an ACCase gene such that the mutated gene encodes a protein comprising at least two amino acid substitutions at the amino acid positions corresponding to D2078G and P2083T of SEQ ID NO: 2.

In some embodiments, a plant or plant cell provided herein may have two or more mutations in an ACCase gene such that the mutated gene encodes a protein comprising at least two amino acid substitutions at the amino acid positions corresponding to D2089 and P2094 of SEQ ID NO: 4 or SEQ ID NO: 954. In some embodiments, the substitution at the amino acid position corresponding to position D2089 of SEQ ID NO: 4 or SEQ ID NO: 954 is selected from the group including, but not limited to: D2089G, D2089K, D2089T, and D2089V. In some embodiments, the substitution at the amino acid position corresponding to position D2089 of SEQ ID NO: 4 or SEQ ID NO: 954 is D2089G. In some embodiments, the substitution at the amino acid position corresponding to position P2094 of SEQ ID NO: 4 or SEQ ID NO: 954 is selected from the group including, but not limited to: P2094T, P2094S, P2094N, and P2094Q. In some embodiments, the substitution at the amino acid position corresponding to position P2094 of SEQ ID NO: 4 or SEQ ID NO: 954 is P2094T. In some embodiments, a plant or plant cell provided herein may have two or more mutations in an ACCase gene such that the mutated gene encodes a protein comprising at least two amino acid substitutions at the amino acid positions corresponding to D2089G and P2094T of SEQ ID NO: 4 or SEQ ID NO: 954.

In some embodiments, a plant or plant cell provided herein may have two or more mutations in an ACCase gene such that the mutated gene encodes a protein comprising at least two amino acid substitutions at the amino acid positions corresponding to D2076 and P2081 of SEQ ID NO: 6. In some embodiments, the substitution at the amino acid position corresponding to position D2076 of SEQ ID NO: 6 is selected from the group including, but not limited to: D2076G, D2076K, D2076T, and D2076V. In some embodiments, the substitution at the amino acid position corresponding to position D2076 of SEQ ID NO: 2 is D2076G. In some embodiments, the substitution at the amino acid position corresponding to position P2081 of SEQ ID NO: 6 is selected from the group including, but not limited to: P2081T, P2081S, P2081N, and P2081Q. In some embodiments, the substitution at the amino acid position corresponding to position P2081 of SEQ ID NO: 6 is P2081T. In some embodiments, a plant or plant cell provided herein may have two or more mutations in an ACCase gene such that the mutated gene encodes a protein comprising at least two amino acid substitutions at the amino acid positions corresponding to D2076G and P2081T of SEQ ID NO: 6.

The present disclosure also relates to a method for producing a plant having a mutated ACCase gene that substantially maintains the catalytic activity of the wild-type protein regardless of the presence or absence of an herbicide of the aryloxyphenoxypropanoate, cyclohexanedione, and/or phenylpyrazoline families. The method may include introducing into a plant cell an ACCase gene that produces a gene product having one or more of the amino acid changes described herein or by mutating or engineering an ACCase gene such that the gene produces a gene product having one or more of the amino acid changes described herein. The method may include introducing into a plant cell an oligonucleobase with a targeted mutation(s) in the ACCase gene that produces a gene product having one or more of the amino acid changes described herein.

The method further includes identifying a cell, seed, or plant having a mutated ACCase gene and to culturing and regeneration methods to obtain a plant that produces seeds, henceforth a “fertile plant”, and the production of seeds and additional plants from such a fertile plant including descendant (progeny) plants that contain the mutated ACCase gene. By way of example, a D2078G mutation (numbering based on SEQ ID NO: 2) can exhibit normal plant growth and strong herbicide tolerance but sterility due to poor pollen viability and inability to produce seeds by self-pollination. A D2078G/P2083T (numbering based on SEQ ID NO: 2) double mutation can exhibit the normal growth rate and herbicide tolerance, but regain fertility.

The disclosure in various embodiments is also directed to novel mutations in the ACCase gene and resulting novel gene product that confer resistance or tolerance to a member of the aryloxyphenoxypropanoate, cyclohexanedione, and/or phenylpyrazoline families, e.g., one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof, to an plant or wherein the mutated ACCase has substantially the same enzymatic activity as compared to wild-type ACCase.

In conjunction with the various aspects, embodiments, compositions, and methods disclosed herein, a plant or plant cell may express a mutated ACCase protein wherein one or more residues of the ACCase gene are changed from the plastidal amino acid residue to the cytosolic amino acid residue.

In a related aspect provided is a plant or plant cell expressing a mutated ACCase protein comprising one or more substitutions at one or more amino acid positions corresponding to a position or position combinations selected from the group consisting of the mutations listed on Table 1 and/or methods of making such a plant and or plant cell. In one embodiment, the ACCase protein comprises a substitution at one or more amino acid positions corresponding to a position or position combinations selected from the group consisting of the substitutions listed on Table 2. In one aspect provided is a plant or plant cell expressing a mutated ACCase protein comprising a substitution at one or more amino acid positions corresponding to a position or position combinations selected from the group consisting of the substitutions listed on Table 3 and/or methods of making such a plant and or plant cell.

In certain embodiments, a plant or plant cell may express a protein comprising one or more substitutions at one or more amino acid positions corresponding to a position selected from the group consisting of N1460, V1529, L1543, L1555, C1575, S1605, G1605-6A*, N1607, L1649, R1672, D1681, F1683, V1715, D1725, R1734, M1738, T1739, A1752, Q1756, I1766, K1772, N1780, I1781, G1783, S1784, A1785, A1786, S1792, I1811, L1818, Q1824, A1837, K1840, V1864, P1870, S1922, K1930, K1938, E1946, A1957, V1966, I1967, M1974, M1975, V1992, W1999, A2004, W2027, E2039, I2041, V2049, A2059, A2067, W2074, V2075, D2078, S2079, K2080, I2081, P2083, R2085, C2088, K2095, G2096, N2097, V2098, E2112, D2127, I2149, E2150, K2154, Q2155, L2159, E2169, A2178, A2179, V2182, F2196, H2223, A2226, K2232, A2241, S2244, F2253, V2254, R2257, N2262, K2264, I2267, S2276, S2280, S2297, L2299, K2302, and K2317 of SEQ ID NO: 2.

In certain embodiments, a plant or plant cell expresses a mutated ACCase protein comprising a substitution at an amino acid position corresponding to a position selected from the group consisting of N1460S, V1529M, L1543F, L1543V, L1555P, C1575S, S1605Y, G1605-6A*, N1607S, L1649M, R1672I, D1681E, D1681G, F1683L, V1715A, D1725E, R1734G, M1738L, T1739S, A1752T, Q1756P, Q1756E, I1766V, K1772E, K1772N, N1780D, I1781L, I1781T, I1781V, I1781A, G1783C, S1784G, S1784N, A1785G, A1786P, S1792L, I1811N, L1818H, L1818I, Q1824P, A1837V, K1840M, K1840R, V1864F, P1870R, S1922G, K1930N, K1938R, E1946G, E1946D, E1946V, A1957T, V1966L, I1967V, M1974T, M1975T, V1992D, W1999C, W1999G, W1999L, W1999S, A2004V, W2027C, W2027R, E2039D, E2039G, I2041D, I2041N, I2041V, V2049F, V2049I, V2049L, V2049T, V2049C, V2049A, V2049S, A2059V, A2067S, W2074L, V2075M, V2075G, V2075I, V2075L, D2078G, D2078K, D2078T, D2078V, S2079F, S2079P, K2080E, P2083N, P2083Q, P2083S, P2083T, R2085S, C2088M, C2088N, C2088R, C2088W, C2088F, C2088G, C2088H, C2088K, C2088S, C2088T, C2088L, C2088V, K2095E, K2095N, G2096A, G2096S, N2097D, N2097S, N2097R, N2097K, N2097E, N2097H, N2097A, N2097G, N2097C, N2097P, N2097L, N2097I, N2097M, N2097F, N2097W, N2097Q, N2097T, N2097Y, V2098A, V2098H, V2098P, V2098S, V2098G, V2098C, E2112G, E2112V, D2127N, I2149K, I2149V, E2150K, A2151T, K2154R, Q2155L, L2159P, E2169D, A2178I, A2179T, V2182A, F2196Y, H2223R, A2226S, K2232N, A2241T, F2253I, V2254I, N2262K, K2264R, I2267V, S2276F, S2276P, S2280P, S2297C, L2299I, K2302Q, and K2317T of SEQ ID NO: 2.

In certain embodiments, a plant or plant cell expresses a mutated ACCase protein comprising one or more substitutions corresponding to one or more substitutions selected from the group consisting of V1529M, L1543F, L1543V, R1672I, D1681E, D1681G, S1784G, S1784N, A1785G, L1818H, L1818I, K1840M, K1840R, A1957T, M1974T, M1975T, D2078G, P2083T, N2097S, V2098G, E2112V, I2149K, A2226S, and N2262K of SEQ ID NO: 2. In certain embodiments, a plant or plant cell expresses a mutated ACCase protein comprising one or more substitutions corresponding to one or more substitutions selected from the group consisting of V1529M, L1543F, L1543V, L1555P, C1575S, S1605Y, G1605-6A*, L1649M, R1672I, D1681E, D1681G, F1683L, V1715A, D1725E, A1752T, I1766V, K1772E, S1784G, S1784N, L1818H, L1818I, K1840M, K1840R, K1930N, A1957T, V1966L, M1974T, M1975T, V1992D, A2067S, R2085S, E2112V, D2127N, I2149K, E2150K, A2151T, K2154R, Q2155L, L2159P, I2267V, E2169D, A2178I, A2179T, V2182A, F2196Y, H2223R, A2226S, K2232N, F2253I, V2254I, N2262K, S2276F, S2276P, S2280P, S2297C, L2299I, K2302Q, and K2317T of SEQ ID NO: 2.

In certain embodiments, a plant or plant cell expresses a mutated ACCase protein comprising an amino acid substitution corresponding to N1460S of SEQ ID NO: 2. In certain embodiments, a plant or plant cell expresses a mutated ACCase protein comprising an amino acid substitution corresponding to V1529M of SEQ ID NO: 2. In certain embodiments, a plant or plant cell expresses expresses a mutated ACCase protein comprising an amino acid substitution corresponding to L1543F of SEQ ID NO: 2. In certain embodiments, a plant or plant cell expresses a mutated ACCase protein comprising an amino acid substitution corresponding to L1543V of SEQ ID NO: 2. In certain embodiments, a plant or plant cell expresses a mutated ACCase protein comprising an amino acid substitution corresponding to L1555P of SEQ ID NO: 2. In certain embodiments, a plant or plant cell expresses a mutated ACCase protein comprising an amino acid substitution corresponding to C1575S of SEQ ID NO: 2. In certain embodiments, a plant or plant cell expresses a mutated ACCase protein comprising an amino acid substitution corresponding to S1605Y of SEQ ID NO: 2. In certain embodiments, a plant or plant cell expresses a mutated ACCase protein comprising an amino acid substitution corresponding to G1605-6A* as described herein. In certain embodiments, a plant or plant cell expresses a mutated ACCase protein comprising an amino acid substitution corresponding to N1607S of SEQ ID NO: 2. In certain embodiments, a plant or plant cell expresses a mutated ACCase protein comprising an amino acid substitution corresponding to L1649M of SEQ ID NO: 2. In certain embodiments, a plant or plant cell expresses a mutated ACCase protein comprising an amino acid substitution corresponding to R1672I of SEQ ID NO: 2. In certain embodiments, a plant or plant cell expresses a mutated ACCase protein comprising an amino acid substitution corresponding to D1681E of SEQ ID NO: 2. In certain embodiments, a plant or plant cell expresses a mutated ACCase protein comprising an amino acid substitution corresponding to D1681G of SEQ ID NO: 2. In certain embodiments, a plant or plant cell expresses a mutated ACCase protein comprising an amino acid substitution corresponding to F1683L of SEQ ID NO: 2. In certain embodiments, a plant or plant cell expresses a mutated ACCase protein comprising an amino acid substitution corresponding to V1715A of SEQ ID NO: 2. In certain embodiments, a plant or plant cell expresses a mutated ACCase protein comprising an amino acid substitution corresponding to D1725E of SEQ ID NO: 2. In certain embodiments, a plant or plant cell expresses a mutated ACCase protein comprising an amino acid substitution corresponding to R1734G of SEQ ID NO: 2. In certain embodiments, a plant or plant cell expresses a mutated ACCase protein comprising an amino acid substitution corresponding to M1738L of SEQ ID NO: 2. In certain embodiments, a plant or plant cell expresses a mutated ACCase protein comprising an amino acid substitution corresponding to T1739S of SEQ ID NO: 2. In certain embodiments, a plant or plant cell expresses a mutated ACCase protein comprising an amino acid substitution corresponding to A1752T of SEQ ID NO: 2. In certain embodiments, a plant or plant cell expresses a mutated ACCase protein comprising an amino acid substitution corresponding to Q1756P of SEQ ID NO: 2. In certain embodiments, a plant or plant cell expresses a mutated ACCase protein comprising an amino acid substitution corresponding to Q1756E of SEQ ID NO: 2. In certain embodiments, a plant or plant cell expresses a mutated ACCase protein comprising an amino acid substitution corresponding to 11766V of SEQ ID NO: 2. In certain embodiments, a plant or plant cell expresses a mutated ACCase protein comprising an amino acid substitution corresponding to K1772E of SEQ ID NO: 2. In certain embodiments, a plant or plant cell expresses a mutated ACCase protein comprising an amino acid substitution corresponding to K1772N of SEQ ID NO: 2. In certain embodiments, a plant or plant cell expresses a mutated ACCase protein comprising an amino acid substitution corresponding to N1780D of SEQ ID NO: 2. In certain embodiments, a plant or plant cell expresses a mutated ACCase protein comprising an amino acid substitution corresponding to I1781L of SEQ ID NO: 2. In certain embodiments, a plant or plant cell expresses a mutated ACCase protein comprising an amino acid substitution corresponding to I1781T of SEQ ID NO: 2. In certain embodiments, a plant or plant cell expresses a mutated ACCase protein comprising an amino acid substitution corresponding to I1781V of SEQ ID NO: 2. In certain embodiments, a plant or plant cell expresses a mutated ACCase protein comprising an amino acid substitution corresponding to I1781A of SEQ ID NO: 2. In certain embodiments, a plant or plant cell expresses a mutated ACCase protein comprising an amino acid substitution corresponding to G1783C of SEQ ID NO: 2. In certain embodiments, a plant or plant cell expresses a mutated ACCase protein comprising an amino acid substitution corresponding to S1784G of SEQ ID NO: 2. In certain embodiments, a plant or plant cell expresses a mutated ACCase protein comprising an amino acid substitution corresponding to S1784N of SEQ ID NO: 2. In certain embodiments, a plant or plant cell expresses a mutated ACCase protein comprising an amino acid substitution corresponding to A1785G of SEQ ID NO: 2. In certain embodiments, a plant or plant cell expresses a mutated ACCase protein comprising an amino acid substitution corresponding to A1786P of SEQ ID NO: 2. In certain embodiments, a plant or plant cell expresses a mutated ACCase protein comprising an amino acid substitution corresponding to S1792L of SEQ ID NO: 2. In certain embodiments, a plant or plant cell expresses a mutated ACCase protein comprising an amino acid substitution corresponding to I1811N of SEQ ID NO: 2. In certain embodiments, a plant or plant cell expresses a mutated ACCase protein comprising an amino acid substitution corresponding to L1818H of SEQ ID NO: 2. In certain embodiments, a plant or plant cell expresses a mutated ACCase protein comprising an amino acid substitution corresponding to L1818I of SEQ ID NO: 2. In certain embodiments, a plant or plant cell expresses a mutated ACCase protein comprising an amino acid substitution corresponding to Q1824P of SEQ ID NO: 2. In certain embodiments, a plant or plant cell expresses a mutated ACCase protein comprising an amino acid substitution corresponding to A1837V of SEQ ID NO: 2. In certain embodiments, a plant or plant cell expresses a mutated ACCase protein comprising an amino acid substitution corresponding to K1840M of SEQ ID NO: 2. In certain embodiments, a plant or plant cell expresses a mutated ACCase protein comprising an amino acid substitution corresponding to K1840R of SEQ ID NO: 2. In certain embodiments, a plant or plant cell expresses a mutated ACCase protein comprising an amino acid substitution corresponding to V1864F of SEQ ID NO: 2. In certain embodiments, a plant or plant cell expresses a mutated ACCase protein comprising an amino acid substitution corresponding to P1870R of SEQ ID NO: 2. In certain embodiments, a plant or plant cell expresses a mutated ACCase protein comprising an amino acid substitution corresponding to S1922G of SEQ ID NO: 2. In certain embodiments, a plant or plant cell expresses a mutated ACCase protein comprising an amino acid substitution corresponding to K1930N, of SEQ ID NO: 2. In certain embodiments, a plant or plant cell expresses a mutated ACCase protein comprising an amino acid substitution corresponding to K1938R of SEQ ID NO: 2. In certain embodiments, a plant or plant cell expresses a mutated ACCase protein comprising an amino acid substitution corresponding to E1946V, of SEQ ID NO: 2. In certain embodiments, a plant or plant cell expresses a mutated ACCase protein comprising an amino acid substitution corresponding to E1946G, of SEQ ID NO: 2. In certain embodiments, a plant or plant cell expresses a mutated ACCase protein comprising an amino acid substitution corresponding to E1946D, of SEQ ID NO: 2. In certain embodiments, a plant or plant cell expresses a mutated ACCase protein comprising an amino acid substitution corresponding to A1957T of SEQ ID NO: 2. In certain embodiments, a plant or plant cell expresses a mutated ACCase protein comprising an amino acid substitution corresponding to V1966L of SEQ ID NO: 2. In certain embodiments, a plant or plant cell expresses a mutated ACCase protein comprising an amino acid substitution corresponding to 11967V of SEQ ID NO: 2. In certain embodiments, a plant or plant cell expresses a mutated ACCase protein comprising an amino acid substitution corresponding to M1974T of SEQ ID NO: 2. In certain embodiments, a plant or plant cell expresses a mutated ACCase protein comprising an amino acid substitution corresponding to M1975T of SEQ ID NO: 2. In certain embodiments, a plant or plant cell expresses a mutated ACCase protein comprising an amino acid substitution corresponding to V1992D of SEQ ID NO: 2. In certain embodiments, a plant or plant cell expresses a mutated ACCase protein comprising an amino acid substitution corresponding to W1999C, of SEQ ID NO: 2. In certain embodiments, a plant or plant cell expresses a mutated ACCase protein comprising an amino acid substitution corresponding to W1999G, of SEQ ID NO: 2. In certain embodiments, a plant or plant cell expresses a mutated ACCase protein comprising an amino acid substitution corresponding to W1999L, of SEQ ID NO: 2. In certain embodiments, a plant or plant cell expresses a mutated ACCase protein comprising an amino acid substitution corresponding to W1999S, of SEQ ID NO: 2. In certain embodiments, a plant or plant cell expresses a mutated ACCase protein comprising an amino acid substitution corresponding to A2004V, of SEQ ID NO: 2. In certain embodiments, a plant or plant cell expresses a mutated ACCase protein comprising an amino acid substitution corresponding to W2027C, of SEQ ID NO: 2. In certain embodiments, a plant or plant cell expresses a mutated ACCase protein comprising an amino acid substitution corresponding to W2027R, of SEQ ID NO: 2. In certain embodiments, a plant or plant cell expresses a mutated ACCase protein comprising an amino acid substitution corresponding to E2039D, of SEQ ID NO: 2. In certain embodiments, a plant or plant cell expresses a mutated ACCase protein comprising an amino acid substitution corresponding to E2039G, of SEQ ID NO: 2. In certain embodiments, a plant or plant cell expresses a mutated ACCase protein comprising an amino acid substitution corresponding to I2041D, of SEQ ID NO: 2. In certain embodiments, a plant or plant cell expresses a mutated ACCase protein comprising an amino acid substitution corresponding to 12041N, of SEQ ID NO: 2. In certain embodiments, a plant or plant cell expresses a mutated ACCase protein comprising an amino acid substitution corresponding to I2041V, of SEQ ID NO: 2. In certain embodiments, a plant or plant cell expresses a mutated ACCase protein comprising an amino acid substitution corresponding to V20491 of SEQ ID NO: 2. In certain embodiments, a plant or plant cell expresses a mutated ACCase protein comprising an amino acid substitution corresponding to V2049F of SEQ ID NO: 2. In certain embodiments, a plant or plant cell expresses a mutated ACCase protein comprising an amino acid substitution corresponding to V2049L of SEQ ID NO: 2. In certain embodiments, a plant or plant cell expresses a mutated ACCase protein comprising an amino acid substitution corresponding to V2049T of SEQ ID NO: 2. In certain embodiments, a plant or plant cell expresses a mutated ACCase protein comprising an amino acid substitution corresponding to V2049C of SEQ ID NO: 2. In certain embodiments, a plant or plant cell expresses a mutated ACCase protein comprising an amino acid substitution corresponding to V2049A of SEQ ID NO: 2. In certain embodiments, a plant or plant cell expresses a mutated ACCase protein comprising an amino acid substitution corresponding to V2049S of SEQ ID NO: 2. In certain embodiments, a plant or plant cell expresses a mutated ACCase protein comprising an amino acid substitution corresponding to A2059V of SEQ ID NO: 2. In certain embodiments, a plant or plant cell expresses a mutated ACCase protein comprising an amino acid substitution corresponding to A2067S of SEQ ID NO: 2. In certain embodiments, a plant or plant cell expresses a mutated ACCase protein comprising an amino acid substitution corresponding to W2074L of SEQ ID NO: 2. In certain embodiments, a plant or plant cell expresses a mutated ACCase protein comprising an amino acid substitution corresponding to V2075I of SEQ ID NO: 2. In certain embodiments, a plant or plant cell expresses a mutated ACCase protein comprising an amino acid substitution corresponding to V2075L of SEQ ID NO: 2. In certain embodiments, a plant or plant cell expresses a mutated ACCase protein comprising an amino acid substitution corresponding to V2075M of SEQ ID NO: 2. In certain embodiments, a plant or plant cell expresses a mutated ACCase protein comprising an amino acid substitution corresponding to V2075G of SEQ ID NO: 2. In certain embodiments, a plant or plant cell expresses a mutated ACCase protein comprising an amino acid substitution corresponding to D2078G of SEQ ID NO: 2. In certain embodiments, a plant or plant cell expresses a mutated ACCase protein comprising an amino acid substitution corresponding to D2078K of SEQ ID NO: 2. In certain embodiments, a plant or plant cell expresses a mutated ACCase protein comprising an amino acid substitution corresponding to D2078T of SEQ ID NO: 2. In certain embodiments, a plant or plant cell expresses a mutated ACCase protein comprising an amino acid substitution corresponding to D2078V of SEQ ID NO: 2. In certain embodiments, a plant or plant cell expresses a mutated ACCase protein comprising an amino acid substitution corresponding to S2079F of SEQ ID NO: 2. In certain embodiments, a plant or plant cell expresses a mutated ACCase protein comprising an amino acid substitution corresponding to S2079P of SEQ ID NO: 2. In certain embodiments, a plant or plant cell expresses a mutated ACCase protein comprising an amino acid substitution corresponding to K2080E of SEQ ID NO: 2. In certain embodiments, a plant or plant cell expresses a mutated ACCase protein comprising an amino acid substitution corresponding to P2083N of SEQ ID NO: 2. In certain embodiments, a plant or plant cell expresses a mutated ACCase protein comprising an amino acid substitution corresponding to P2083Q of SEQ ID NO: 2. In certain embodiments, a plant or plant cell expresses a mutated ACCase protein comprising an amino acid substitution corresponding to P2083S of SEQ ID NO: 2. In certain embodiments, a plant or plant cell expresses a mutated ACCase protein comprising an amino acid substitution corresponding to P2083T of SEQ ID NO: 2. In certain embodiments, a plant or plant cell expresses a mutated ACCase protein comprising an amino acid substitution corresponding to R2085S of SEQ ID NO: 2. In certain embodiments, a plant or plant cell expresses a mutated ACCase protein comprising an amino acid substitution corresponding to C2088M of SEQ ID NO: 2. In certain embodiments, a plant or plant cell expresses a mutated ACCase protein comprising an amino acid substitution corresponding to C2088N of SEQ ID NO: 2. In certain embodiments, a plant or plant cell expresses a mutated ACCase protein comprising an amino acid substitution corresponding to C2088R of SEQ ID NO: 2. In certain embodiments, a plant or plant cell expresses a mutated ACCase protein comprising an amino acid substitution corresponding to C2088W of SEQ ID NO: 2. In certain embodiments, a plant or plant cell expresses a mutated ACCase protein comprising an amino acid substitution corresponding to C2088F of SEQ ID NO: 2. In certain embodiments, a plant or plant cell expresses a mutated ACCase protein comprising an amino acid substitution corresponding to C2088G of SEQ ID NO: 2. In certain embodiments, a plant or plant cell expresses a mutated ACCase protein comprising an amino acid substitution corresponding to C2088H of SEQ ID NO: 2. In certain embodiments, a plant or plant cell expresses a mutated ACCase protein comprising an amino acid substitution corresponding to C2088K of SEQ ID NO: 2. In certain embodiments, a plant or plant cell expresses a mutated ACCase protein comprising an amino acid substitution corresponding to C2088S of SEQ ID NO: 2. In certain embodiments, a plant or plant cell expresses a mutated ACCase protein comprising an amino acid substitution corresponding to C2088T of SEQ ID NO: 2. In certain embodiments, a plant or plant cell expresses a mutated ACCase protein comprising an amino acid substitution corresponding to C2088L of SEQ ID NO: 2. In certain embodiments, a plant or plant cell expresses a mutated ACCase protein comprising an amino acid substitution corresponding to C2088V of SEQ ID NO: 2. In certain embodiments, a plant or plant cell expresses a mutated ACCase protein comprising an amino acid substitution corresponding to K2095E of SEQ ID NO: 2. In certain embodiments, a plant or plant cell expresses a mutated ACCase protein comprising an amino acid substitution corresponding to K2095N of SEQ ID NO: 2. In certain embodiments, a plant or plant cell expresses a mutated ACCase protein comprising an amino acid substitution corresponding to G2096A of SEQ ID NO: 2. In certain embodiments, a plant or plant cell expresses a mutated ACCase protein comprising an amino acid substitution corresponding to G2096S of SEQ ID NO: 2. In certain embodiments, a plant or plant cell expresses a mutated ACCase protein comprising an amino acid substitution corresponding to N2097D of SEQ ID NO: 2. In certain embodiments, a plant or plant cell expresses a mutated ACCase protein comprising an amino acid substitution corresponding to N2097S, of SEQ ID NO: 2. In certain embodiments, a plant or plant cell expresses a mutated ACCase protein comprising an amino acid substitution corresponding to N2097R, of SEQ ID NO: 2. In certain embodiments, a plant or plant cell expresses a mutated ACCase protein comprising an amino acid substitution corresponding to N2097K, of SEQ ID NO: 2. In certain embodiments, a plant or plant cell expresses a mutated ACCase protein comprising an amino acid substitution corresponding to N2097E, of SEQ ID NO: 2. In certain embodiments, a plant or plant cell expresses a mutated ACCase protein comprising an amino acid substitution corresponding to N2097H, of SEQ ID NO: 2. In certain embodiments, a plant or plant cell expresses a mutated ACCase protein comprising an amino acid substitution corresponding to N2097A, of SEQ ID NO: 2. In certain embodiments, a plant or plant cell expresses a mutated ACCase protein comprising an amino acid substitution corresponding to N2097G, of SEQ ID NO: 2. In certain embodiments, a plant or plant cell expresses a mutated ACCase protein comprising an amino acid substitution corresponding to N2097C, of SEQ ID NO: 2. In certain embodiments, a plant or plant cell expresses a mutated ACCase protein comprising an amino acid substitution corresponding to N2097P, of SEQ ID NO: 2. In certain embodiments, a plant or plant cell expresses a mutated ACCase protein comprising an amino acid substitution corresponding to N2097L, of SEQ ID NO: 2. In certain embodiments, a plant or plant cell expresses a mutated ACCase protein comprising an amino acid substitution corresponding to N2097I, of SEQ ID NO: 2. In certain embodiments, a plant or plant cell expresses a mutated ACCase protein comprising an amino acid substitution corresponding to N2097M, of SEQ ID NO: 2. In certain embodiments, a plant or plant cell expresses a mutated ACCase protein comprising an amino acid substitution corresponding to N2097F, of SEQ ID NO: 2. In certain embodiments, a plant or plant cell expresses a mutated ACCase protein comprising an amino acid substitution corresponding to N2097W, of SEQ ID NO: 2. In certain embodiments, a plant or plant cell expresses a mutated ACCase protein comprising an amino acid substitution corresponding to N2097Q, of SEQ ID NO: 2. In certain embodiments, a plant or plant cell expresses a mutated ACCase protein comprising an amino acid substitution corresponding to N2097T, of SEQ ID NO: 2. In certain embodiments, a plant or plant cell expresses a mutated ACCase protein comprising an amino acid substitution corresponding to N2097Y, of SEQ ID NO: 2. In certain embodiments, a plant or plant cell expresses a mutated ACCase protein comprising an amino acid substitution corresponding to V2098A of SEQ ID NO: 2. In certain embodiments, a plant or plant cell expresses a mutated ACCase protein comprising an amino acid substitution corresponding to V2098G, of SEQ ID NO: 2. In certain embodiments, a plant or plant cell expresses a mutated ACCase protein comprising an amino acid substitution corresponding to V2098H, of SEQ ID NO: 2. In certain embodiments, a plant or plant cell expresses a mutated ACCase protein comprising an amino acid substitution corresponding to V2098P, of SEQ ID NO: 2. In certain embodiments, a plant or plant cell expresses a mutated ACCase protein comprising an amino acid substitution corresponding to V2098S, of SEQ ID NO: 2. In certain embodiments, a plant or plant cell expresses a mutated ACCase protein comprising an amino acid substitution corresponding to V2098C, of SEQ ID NO: 2. In certain embodiments, a plant or plant cell expresses a mutated ACCase protein comprising an amino acid substitution corresponding to E2112G, of SEQ ID NO: 2. In certain embodiments, a plant or plant cell expresses a mutated ACCase protein comprising an amino acid substitution corresponding to E2112V of SEQ ID NO: 2. In certain embodiments, a plant or plant cell expresses a mutated ACCase protein comprising an amino acid substitution corresponding to D2127N of SEQ ID NO: 2. In certain embodiments, a plant or plant cell expresses a mutated ACCase protein comprising an amino acid substitution corresponding to 12149K of SEQ ID NO: 2. In certain embodiments, a plant or plant cell expresses a mutated ACCase protein comprising an amino acid substitution corresponding to 12149V, of SEQ ID NO: 2. In certain embodiments, a plant or plant cell expresses a mutated ACCase protein comprising an amino acid substitution corresponding to E2150K of SEQ ID NO: 2. In certain embodiments, a plant or plant cell expresses a mutated ACCase protein comprising an amino acid substitution corresponding to A2151T of SEQ ID NO: 2. In certain embodiments, a plant or plant cell expresses a mutated ACCase protein comprising an amino acid substitution corresponding to K2154R of SEQ ID NO: 2. In certain embodiments, a plant or plant cell expresses a mutated ACCase protein comprising an amino acid substitution corresponding to Q2155L of SEQ ID NO: 2. In certain embodiments, a plant or plant cell expresses a mutated ACCase protein comprising an amino acid substitution corresponding to L2159P of SEQ ID NO: 2. In certain embodiments, a plant or plant cell expresses a mutated ACCase protein comprising an amino acid substitution corresponding to E2169D of SEQ ID NO: 2. In certain embodiments, a plant or plant cell expresses a mutated ACCase protein comprising an amino acid substitution corresponding to A21781 of SEQ ID NO: 2. In certain embodiments, a plant or plant cell expresses a mutated ACCase protein comprising an amino acid substitution corresponding to A2179T of SEQ ID NO: 2. In certain embodiments, a plant or plant cell expresses a mutated ACCase protein comprising an amino acid substitution corresponding to V2182A of SEQ ID NO: 2. In certain embodiments, a plant or plant cell expresses a mutated ACCase protein comprising an amino acid substitution corresponding to F2196Y of SEQ ID NO: 2. In certain embodiments, a plant or plant cell expresses a mutated ACCase protein comprising an amino acid substitution corresponding to H2223R of SEQ ID NO: 2. In certain embodiments, a plant or plant cell expresses a mutated ACCase protein comprising an amino acid substitution corresponding to A2226S of SEQ ID NO: 2. In certain embodiments, a plant or plant cell expresses a mutated ACCase protein comprising an amino acid substitution corresponding to K2232N of SEQ ID NO: 2. In certain embodiments, a plant or plant cell expresses a mutated ACCase protein comprising an amino acid substitution corresponding to A2241T, of SEQ ID NO: 2. In certain embodiments, a plant or plant cell expresses a mutated ACCase protein comprising an amino acid substitution corresponding to F2253I of SEQ ID NO: 2. In certain embodiments, a plant or plant cell expresses a mutated ACCase protein comprising an amino acid substitution corresponding to V2254I of SEQ ID NO: 2. In certain embodiments, a plant or plant cell expresses a mutated ACCase protein comprising an amino acid substitution corresponding to N2262K of SEQ ID NO: 2. In certain embodiments, a plant or plant cell expresses a mutated ACCase protein comprising an amino acid substitution corresponding to K2264R, of SEQ ID NO: 2. In certain embodiments, a plant or plant cell expresses a mutated ACCase protein comprising an amino acid substitution corresponding to I2267V of SEQ ID NO: 2. In certain embodiments, a plant or plant cell expresses a mutated ACCase protein comprising an amino acid substitution corresponding to S2276F of SEQ ID NO: 2. In certain embodiments, a plant or plant cell expresses a mutated ACCase protein comprising an amino acid substitution corresponding to S2276P of SEQ ID NO: 2. In certain embodiments, a plant or plant cell expresses a mutated ACCase protein comprising an amino acid substitution corresponding to S2280P of SEQ ID NO: 2. In certain embodiments, a plant or plant cell expresses a mutated ACCase protein comprising an amino acid substitution corresponding to S2297C of SEQ ID NO: 2. In certain embodiments, a plant or plant cell expresses a mutated ACCase protein comprising an amino acid substitution corresponding to L22991 of SEQ ID NO: 2. In certain embodiments, a plant or plant cell expresses a mutated ACCase protein comprising an amino acid substitution corresponding to K2302Q of SEQ ID NO: 2. In certain embodiments, a plant or plant cell expresses a mutated ACCase protein comprising an amino acid substitution corresponding to K2317T of SEQ ID NO: 2.

In certain embodiments, a plant or plant cell expresses a mutated ACCase protein comprising an amino acid substitution corresponding to N1460S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V1529M of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to L1543F of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to L1543V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to L1555P of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C1575S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S1605Y of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to G1605-6A* of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N1607S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to L1649M of SEQ ID NO: 2 and an additional substitution or substitution combination such as provided herein (for example a substitution embodiments, a plant or plant cell expresses a mutated ACCase protein comprising an amino acid substitution corresponding to R1672I of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to D1681E of SEQ ID NO: 2. In certain embodiments, a plant or plant cell expresses a mutated ACCase protein comprising an amino acid substitution corresponding to D1681G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to F1683L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V1715A of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to D1725E of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to R1734G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to M1738L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to T1739S of SEQ ID NO: 2 and an additional substitution or substitution combination such as provided herein (for example a substitution embodiments, a plant or plant cell expresses a mutated ACCase protein comprising an amino acid substitution corresponding to A1752T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to Q1756P of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to Q1756E of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to 11766V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K1772E of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K1772N of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N1780D of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to I1781L of SEQ ID NO: 2. In certain embodiments, a plant or plant cell expresses a mutated ACCase protein comprising an amino acid substitution corresponding to I1781T of SEQ ID NO: 2 and an additional substitution or substitution combination such as provided herein (for example a substitution embodiments, a plant or plant cell expresses a mutated ACCase protein comprising an amino acid substitution corresponding to I1781V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to I1781A of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to G1783C of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S1784G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S1784N of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A1785G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A1786P of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S1792L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to I1811N of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to L1818H of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to L1818I of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to Q1824P of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A1837V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K1840M of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K1840R of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V1864F of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to P1870R of SEQ ID NO: 2 and an additional substitution or substitution combination such as provided herein (for example a substitution embodiments, a plant or plant cell expresses a mutated ACCase protein comprising an amino acid substitution corresponding to S1922G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K1930N, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K1938R of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E1946G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E1946D of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E1946V, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A1957T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V1966L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to I1967V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to M1974T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to M1975T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V1992D of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to W1999C of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to W1999G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to W1999L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to W1999S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A2004V of SEQ ID NO: 2 and an additional substitution or substitution combination such as provided herein (for example a substitution embodiments, a plant or plant cell expresses a mutated ACCase protein comprising an amino acid substitution corresponding to W2027C of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to W2027R of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E2039D of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E2039G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to I2041D of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to I2041N of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to 12041V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2049F of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V20491 of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2049L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2049T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2049C of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2049A of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2049S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A2059V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A2067S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A2074L of SEQ ID NO: 2 and an additional substitution or substitution combination such as provided herein (for example a substitution embodiments, a plant or plant cell expresses a mutated ACCase protein comprising an amino acid substitution corresponding to V2075G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2075M of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2075I of SEQ ID NO: 2 and an additional substitution or substitution combination such as provided herein (for example a substitution or substitution combination provided in Table 1, Table 2 or Table 3). In certain embodiments, a plant or plant cell expresses a mutated ACCase protein comprising an amino acid substitution corresponding to V2075L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to D2078G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to D2078K of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to D2078T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to D2078V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S2079F of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S2079P of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K2080E of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to P2083N of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to P2083Q of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to P2083S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to P2083T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to R2085S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088M of SEQ ID NO: 2 and an additional substitution or substitution combination such as provided herein (for example a substitution embodiments, a plant or plant cell expresses a mutated ACCase protein comprising an amino acid substitution corresponding to C2088N of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088R of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088W of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088F of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088G of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088H of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088K of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to C2088V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K2095E of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K2095N of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to G2096A of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to G2096S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097D of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097S, of SEQ ID NO: 2 and an additional substitution or substitution combination such as provided herein (for example a substitution embodiments, a plant or plant cell expresses a mutated ACCase protein comprising an amino acid substitution corresponding to N2097R, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097K, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097E, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097H, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097A, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097G, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097C, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097P, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097L, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097I, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097M, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097F, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097W, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097Q, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097T, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2097Y, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2098A of SEQ ID NO: 2 and an additional substitution or substitution combination such as provided herein (for example a substitution embodiments, a plant or plant cell expresses a mutated ACCase protein comprising an amino acid substitution corresponding to V2098G, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2098H, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2098P, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2098S, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2098C, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E2112G, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E2112V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to D2127N of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to I2149K of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E2150K of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A2151T of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K2154R of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to Q2155L of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to L2159P of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to E2169D of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A21781 of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A2179T of SEQ ID NO: 2 and an additional substitution or substitution combination such as provided herein (for example a substitution embodiments, a plant or plant cell expresses a mutated ACCase protein comprising an amino acid substitution corresponding to V2182A of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to F2196Y of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to H2223R of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to A2226S of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K2232N of SEQ ID NO: 2. In certain embodiments, a plant or plant cell expresses a mutated ACCase protein comprising an amino acid substitution corresponding to A2241T, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to F2253I of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to V2254I of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to N2262K of SEQ ID NO: 2 and an additional substitution or substitution combination such as provided herein (for example a substitution embodiments, a plant or plant cell expresses a mutated ACCase protein comprising an amino acid substitution corresponding to K2264R, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to I2267V of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S2276F, of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S2276P of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S2280P of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to S2297C of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to L22991 of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K2302Q of SEQ ID NO: 2 and an additional substitution or substitution combination such as provided herein (for example a substitution or substitution combination provided in Table 1, Table 2, or Table 3). In certain embodiments, a plant or plant cell expresses a mutated ACCase protein comprising an amino acid substitution corresponding to K2317T of SEQ ID NO: 2 and an additional substitution or substitution combination such as provided herein (for example a substitution or substitution combination provided in Table 1, Table 2, or Table 3).

In certain embodiments, a plant or plant cell provided herein expresses a mutated ACCase protein comprising an amino acid substitution at a position corresponding to D2078 of SEQ ID NO: 2 (i.e., wherein the encoded protein has any amino acid other than an aspartate at the position corresponding to D2078 of SEQ ID NO: 2). In certain embodiments, a plant or plant cell expresses a mutated ACCase protein comprising an amino acid substitution at a position corresponding to D2078G of SEQ ID NO: 2. In certain embodiments, a plant or plant cell expresses a mutated ACCase protein comprising an amino acid substitution at a position corresponding to D2078K of SEQ ID NO: 2. In certain embodiments, a plant or plant cell expresses a mutated ACCase protein comprising an amino acid substitution at a position corresponding to D2078T of SEQ ID NO: 2. In certain embodiments, a plant or plant cell expresses a mutated ACCase protein comprising an amino acid substitution at a position corresponding to D2078V of SEQ ID NO: 2. In some embodiments, a plant or plant cell provided herein expresses a mutated ACCase protein comprising an amino acid substitution at a position corresponding to D2078 of SEQ ID NO: 2 (such as a substitution as provided herein) in combination with an additional ACCase mutation or mutations as provided herein. In some embodiments, a plant or plant cell provided herein expresses a mutated ACCase protein comprising an amino acid substitution at a position corresponding to D2078 of SEQ ID NO: 2 (such as a substitution as provided herein) in combination with an additional ACCase mutation or mutations as provided in Table 1. In some embodiments, a plant or plant cell provided herein expresses a mutated ACCase protein comprising an amino acid substitution at a position corresponding to D2078 of SEQ ID NO: 2 (such as a substitution as provided herein) in combination with an additional ACCase mutation or mutations as provided in Table 2. In some embodiments, a plant or plant cell provided herein expresses a mutated ACCase protein comprising an amino acid substitution at a position corresponding to D2078 of SEQ ID NO: 2 (such as a substitution as provided herein) in combination with an additional ACCase mutation as provided in Table 3. In some embodiments, a plant or plant cell provided herein expresses a mutated ACCase protein comprising an amino acid substitution at a position corresponding to D2078 of SEQ ID NO: 2 (such as a substitution as provided herein) in combination with an additional ACCase mutation at an amino acid position corresponding to P2083 of SEQ ID NO: 2.

In certain embodiments, a plant or plant cell provided herein expresses a mutated ACCase protein comprising a substitution at an amino acid position corresponding to P2083 of SEQ ID NO: 2 (i.e., wherein the encoded protein has any amino acid other than a proline at the position corresponding to P2083 of SEQ ID NO: 2). In certain embodiments, a plant or plant cell expresses a mutated ACCase protein comprising an amino acid substitution at a position corresponding to P2083N of SEQ ID NO: 2. In certain embodiments, a plant or plant cell expresses a mutated ACCase protein comprising an amino acid substitution at a position corresponding to P2083Q of SEQ ID NO: 2. In certain embodiments, a plant or plant cell expresses a mutated ACCase protein comprising an amino acid substitution at a position corresponding to P2083S of SEQ ID NO: 2. In certain embodiments, a plant or plant cell expresses a mutated ACCase protein comprising an amino acid substitution at a position corresponding to P2083T of SEQ ID NO: 2. In some embodiments, a plant or plant cell provided herein expresses a mutated ACCase protein comprising a substitution at an amino acid position corresponding to P2083 of SEQ ID NO: 2 (such as a substitution as provided herein) in combination with an additional ACCase mutation or mutations as provided herein. In some embodiments, a plant or plant cell provided herein expresses a mutated ACCase protein comprising a substitution at an amino acid position corresponding to P2083 of SEQ ID NO: 2 (such as a substitution as provided herein) in combination with an additional ACCase mutation or mutations as provided in Table 1. In some embodiments, a plant or plant cell provided herein expresses a mutated ACCase protein comprising a substitution at an amino acid position corresponding to P2083 of SEQ ID NO: 2 (such as a substitution as provided herein) in combination with an additional ACCase mutation or mutations as provided in Table 2. In some embodiments, a plant or plant cell provided herein expresses a mutated ACCase protein comprising a substitution at an amino acid position corresponding to P2083 of SEQ ID NO: 2 (such as a substitution as provided herein) in combination with an additional ACCase mutation as provided in Table 3. In some embodiments, a plant or plant cell provided herein expresses a mutated ACCase protein comprising a substitution at an amino acid position corresponding to P2083 of SEQ ID NO: 2 (such as a substitution as provided herein) in combination with an additional ACCase mutation at an amino acid position corresponding to D2078 of SEQ ID NO: 2.

In some embodiments, a plant or plant cell provided herein expresses a protein comprising at least two amino acid substitutions at the amino acid positions corresponding to D2078 and P2083 of SEQ ID NO: 2. In some embodiments, the substitution at the amino acid position corresponding to position D2078 of SEQ ID NO: 2 is selected from the group including, but not limited to: D2078G, D2078K, D2078T, and D2078V. In some embodiments, the substitution at the amino acid position corresponding to position D2078 of SEQ ID NO: 2 is D2078G. In some embodiments, the substitution at the amino acid position corresponding to position P2083 of SEQ ID NO: 2 is selected from the group including, but not limited to: P2083T, P2083S, P2083N, and P2083Q. In some embodiments, the substitution at the amino acid position corresponding to position P2083 of SEQ ID NO: 2 is P2083T. In some embodiments, a plant or plant cell provided herein expresses a protein comprising at least two amino acid substitutions at the amino acid positions corresponding to D2078G and P2083T of SEQ ID NO: 2.

In some embodiments, a plant or plant cell provided herein expresses a protein comprising at least two amino acid substitutions at the amino acid positions corresponding to D2089 and P2094 of SEQ ID NO: 4 or SEQ ID NO: 954. In some embodiments, the substitution at the amino acid position corresponding to position D2089 of SEQ ID NO: 4 or SEQ ID NO: 954 is selected from the group including, but not limited to: D2089G, D2089K, D2089T, and D2089V. In some embodiments, the substitution at the amino acid position corresponding to position D2089 of SEQ ID NO: 4 or SEQ ID NO: 954 is D2089G. In some embodiments, the substitution at the amino acid position corresponding to position P2094 of SEQ ID NO: 4 or SEQ ID NO: 954 is selected from the group including, but not limited to: P2094T, P2094S, P2094N, and P2094Q. In some embodiments, the substitution at the amino acid position corresponding to position P2094 of SEQ ID NO: 4 or SEQ ID NO: 954 is P2094T. In some embodiments, a plant or plant cell provided herein expresses a protein comprising at least two amino acid substitutions at the amino acid positions corresponding to D2089G and P2094T of SEQ ID NO: 4 or SEQ ID NO: 954.

In some embodiments, a plant or plant cell provided herein expresses a protein comprising at least two amino acid substitutions at the amino acid positions corresponding to D2076 and P2081 of SEQ ID NO: 6. In some embodiments, the substitution at the amino acid position corresponding to position D2076 of SEQ ID NO: 6 is selected from the group including, but not limited to: D2076G, D2076K, D2076T, and D2076V. In some embodiments, the substitution at the amino acid position corresponding to position D2076 of SEQ ID NO: 2 is D2076G. In some embodiments, the substitution at the amino acid position corresponding to position P2081 of SEQ ID NO: 6 is selected from the group including, but not limited to: P2081T, P2081S, P2081N, and P2081Q. In some embodiments, the substitution at the amino acid position corresponding to position P2081 of SEQ ID NO: 6 is P2081T. In some embodiments, a plant or plant cell provided herein expresses a protein comprising at least two amino acid substitutions at the amino acid positions corresponding to D2076G and P2081T of SEQ ID NO: 6.

The present disclosure also relates to a method for producing a plant having a mutated ACCase gene that substantially maintains the catalytic activity of the wild-type protein regardless of the presence or absence of an herbicide of the aryloxyphenoxypropanoate, cyclohexanedione, and/or phenylpyrazoline families. The method may include introducing into a plant cell an ACCase gene that produces a gene product having one or more of the amino acid changes described herein or by mutating or engineering an ACCase gene such that the gene produces a gene product having one or more of the amino acid changes described herein. The method may include introducing into a plant cell an oligonucleobase with a targeted mutation(s) in the ACCase gene that produces a gene product having one or more of the amino acid changes described herein. The method further includes identifying a cell, seed, or plant having a mutated ACCase gene and to culturing and regeneration methods to obtain a plant that produces seeds, henceforth a “fertile plant”, and the production of seeds and additional plants from such a fertile plant including descendant (progeny) plants that contain the mutated ACCase gene.

The present disclosure also relates to modified ACCase proteins as described herein, nucleic acides encoding such proteins, and methods of expressing such proteins.

In certain embodiments, a mutated plant as provided herein is resistant to one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof, and in certain aspects retains substantially the same catalytic activity as compared to the wild-type corresponding ACCase protein.

The disclosure is further directed to a method of selectively controlling weeds in a field. The field may include plants with the disclosed ACCase gene alterations. The method comprises application to the field of an herbicide of the aryloxyphenoxypropanoate, cyclohexanedione, and/or phenylpyrazoline families to which the plants are rendered resistant, and the weeds are controlled. A preferred herbicide may be one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof.

### BRIEF DESCRIPTION OF THE FIGURES

FIG. 1A depicts a cDNA sequence of Alopecurus myosuroides plastidal ACCase AMY310767 (SEQ ID NO: 1).

FIG. 1B depicts an amino acid sequence of Alopecurus myosuroides plastidal ACCase AMY310767 (SEQ ID NO: 2).

FIG. 2A depicts a cDNA sequence of Oryza sativa (Cyprus) plastidal ACCase Os05g22940 (SEQ ID NO: 3).

FIG. 2B depicts an amino acid sequence of Oryza sativa (Cyprus) plastidal ACCase Os05g22940 (SEQ ID NO: 4).

FIG. 2C depicts a genomic DNA-reverse complemented fragment sequence of Oryza sativa (Cyprus) plastidal ACCase Os05g22940 (SEQ ID NO: 5).

FIG. 3 depicts an amino acid sequence of Oryza sativa (Japonica) plastidal ACCase Os05g22940 (SEQ ID NO: 6).

FIG. 4A depicts a cDNA sequence of Triticum aestivum plastidal ACCase AF29895 (SEQ ID NO: 7).

FIG. 4B depicts an amino acid sequence of Triticum aestivum plastidal ACCase AF29895 (SEQ ID NO: 8).

FIG. 5A depicts a cDNA sequence of Zea mays plastidal ACCase 1a AY312171 (SEQ ID NO: 9).

FIG. 5B depicts an amino acid sequence of Zea mays plastidal ACCase 1a AY312171 (SEQ ID NO: 10).

FIG. 6A depicts a cDNA sequence of Zea mays plastidal ACCase 1b AY312172 (SEQ ID NO: 11).

FIG. 6B depicts an amino acid sequence of Zea mays plastidal ACCase 1b AY312172 (SEQ ID NO: 12.

FIG. 7A depicts a cDNA sequence of Zea mays plastidal ACCase ZMU19183 (SEQ ID NO: 13).

FIG. 7B depicts an amino acid sequence of Zea mays plastidal ACCase ZMU19183 (SEQ ID NO: 14).

FIG. 8A depicts a cDNA sequence of Setaria italica plastidal ACCase AY219174 (SEQ ID NO: 15).

FIG. 8B depicts an amino acid sequence of Setaria italica plastidal ACCase AY219174 (SEQ ID NO: 16).

FIG. 9A depicts a cDNA sequence of Setaria italica plastidal ACCase AY219175 DIM tolerant (SEQ ID NO: 17).

FIG. 9B depicts an amino acid sequence of Setaria italica plastidal ACCase AY219175 DIM tolerant (SEQ ID NO: 18).

FIG. 10A depicts a cDNA sequence of Setaria italica plastidal ACCase AF294805 (SEQ ID NO: 19).

FIG. 10B depicts an amino acid sequence of Setaria italica plastidal ACCase AF294805 (SEQ ID NO: 20).

FIG. 11A depicts a cDNA sequence of Sorghum bicolor plastidal ACCase Sb06003090 (SEQ ID NO: 21).

FIG. 11B depicts an amino acid sequence of Sorghum bicolor plastidal ACCase Sb06003090 (SEQ ID NO: 22).

FIG. 12 depicts an amino acid sequence of Brachypodium distachyon plastidal ACCase Bradi5g03860 (SEQ ID NO: 23).

FIG. 13A depicts a cDNA sequence of Zea mays plastidal ACCase NC050105.1 (SEQ ID NO: 950)

FIG. 13B depicts an amino acid sequence of Zea mays plastidal ACCase NC050105.1 (SEQ ID NO: 951)

FIG. 14A depicts a cDNA sequence of Sorghum bicolor plastidal ACCase NC012875 (SEQ ID NO: 952)

FIG. 14B depicts an amino acid sequence of Sorghum bicolor plastidal ACCase NC012875 (SEQ ID NO: 953)

FIG. 15 depicts an amino acid sequence of Oryza sativa subsp. Japonica plastidal ACCase NP001407229.1 (SEQ ID NO: 954)

FIG. 16A depicts a cDNA sequence of Hordeum vulgare plastidal ACCase NC058519.1 (SEQ ID NO: 955)

FIG. 16B depicts an amino acid sequence of Hordeum vulgare plastidal ACCase NC058519.1 (SEQ ID NO: 956)

FIG. 17A depicts a cDNA sequence of Triticum aestivum plastidal ACCase NC057799.1 (SEQ ID NO: 957)

FIG. 17B depicts an amino acid sequence of Triticum aestivum plastidal ACCase NC057799.1 (SEQ ID NO: 958)

FIG. 18A depicts a cDNA sequence of Triticum aestivum plastidal ACCase NC057798.1 (SEQ ID NO: 959)

FIG. 18B depicts an amino acid sequence of Triticum aestivum plastidal ACCase NC057798.1 (SEQ ID NO: 960)

FIG. 19A depicts a cDNA sequence of Triticum aestivum plastidal ACCase NC057797.1 (SEQ ID NO: 961)

FIG. 19B depicts an amino acid sequence of Triticum aestivum plastidal ACCase NC057797.1 (SEQ ID NO: 962)

FIG. 20A depicts a cDNA sequence of Setaria italica plastidal ACCase NC028456.1 (SEQ ID NO: 963)

FIG. 20B depicts an amino acid sequence of Setaria italica plastidal ACCase NC028456.1 (SEQ ID NO: 964)

FIG. 21A depicts a cDNA sequence of Lolium perenne plastidal ACCase NC067245.1 (SEQ ID NO: 965)

FIG. 21B depicts an amino acid sequence of Lolium perenne plastidal ACCase NC067245.1 (SEQ ID NO: 966)

FIG. 22A depicts a cDNA sequence of Panicum virgatum plastidal ACCase NC053142.1 (SEQ ID NO: 967)

FIG. 22B depicts an amino acid sequence of Panicum virgatum plastidal ACCase NC053142.1 (SEQ ID NO: 968)

FIG. 23A depicts a cDNA sequence of Avena sativa plastidal ACCase PepsiCoV2 chr4C (SEQ ID NO: 969)

FIG. 23B depicts an amino acid sequence of Avena sativa plastidal ACCase PepsiCoV2 chr4C (SEQ ID NO: 970)

FIG. 24A depicts a cDNA sequence of Avena sativa plastidal ACCase PepsiCoV2 chr5D (SEQ ID NO: 971)

FIG. 24B depicts an amino acid sequence of Avena sativa plastidal ACCase PepsiCoV2 chr5D (SEQ ID NO: 972)

FIG. 25A depicts a cDNA sequence of Avena sativa plastidal ACCase PepsiCoV2 chr6A (SEQ ID NO: 973)

FIG. 25B depicts an amino acid sequence of Avena sativa plastidal ACCase PepsiCoV2 chr6A (SEQ ID NO: 974)

FIG. 26A depicts a cDNA sequence of Poa pratensis plastidal ACCase CAMZMU010000063 (SEQ ID NO: 975)

FIG. 26B depicts an amino acid sequence of Poa pratensis plastidal ACCase CAMZMU010000063 (SEQ ID NO: 976)

FIG. 27A depicts a cDNA sequence of Poa pratensis plastidal ACCase CAMZMU010000098 (SEQ ID NO: 977)

FIG. 27B depicts an amino acid sequence of Poa pratensis plastidal ACCase CAMZMU010000098 (SEQ ID NO: 978)

### DETAILED DESCRIPTION

In various aspects and embodiments of the present disclosure, provided include a plant or plant cell having one or more ACCase mutations and/or mutation combinations, methods of making such a plant or plant cell, and methods for producing plants having desirable clethodim tolerance. In various aspects and embodiments of the present disclosure, provided include a plant or plant cell having one or more ACCase mutations and/or mutation combinations, methods of making such a plant or plant cell, and methods for producing plants having desirable fertility.

It will be readily apparent to a person skilled in the art that varying substitutions and modifications may be made to this disclosure without departing from the scope and spirit of the disclosure.

The disclosure illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

Thus, it should be understood that although the present disclosure has been specifically disclosed by preferred embodiments and optional features, modification, improvement, and variation of the disclosures disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### Acetyl-CoA Carboxylase (ACCase) Genes

The present disclosure generally relates to plants having mutations in acetyl-CoA carboxylase (ACCase) genes. In some embodiments, one or more mutations in an ACCase gene results in increased herbicide resistance or tolerance. In some embodiments, one or more mutations in an ACCase gene results in increased clethodim resistance or tolerance. A set of exemplary nucleic acid and corresponding amino acid sequences are provided in the Figures.

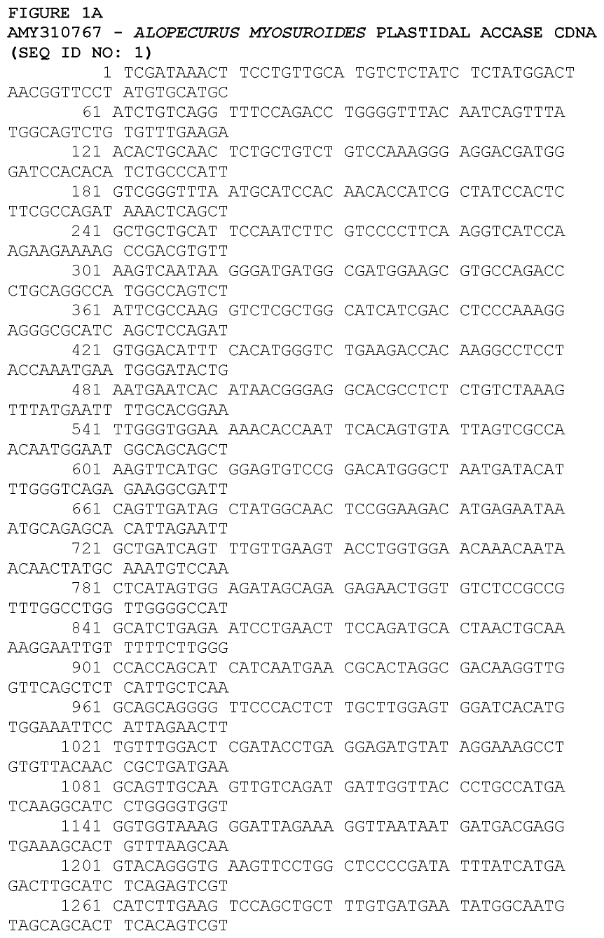

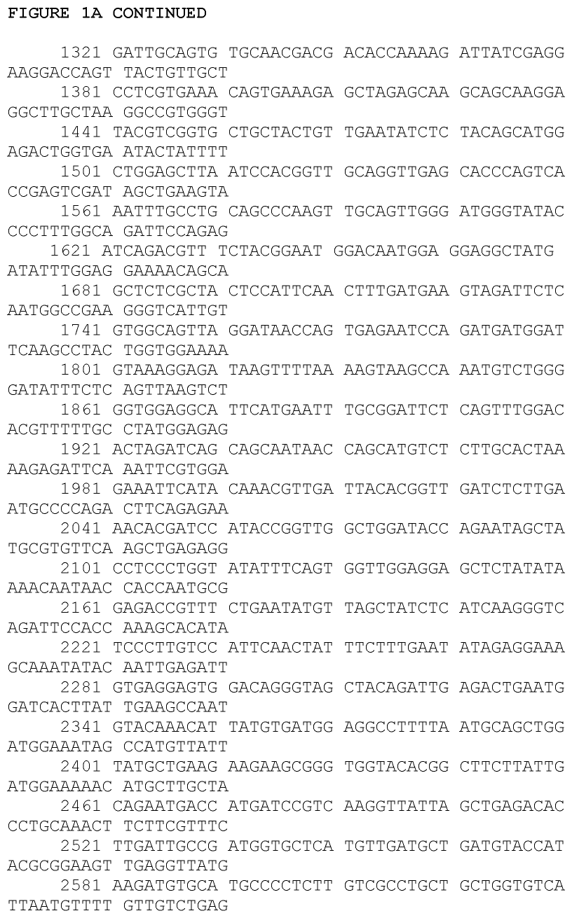

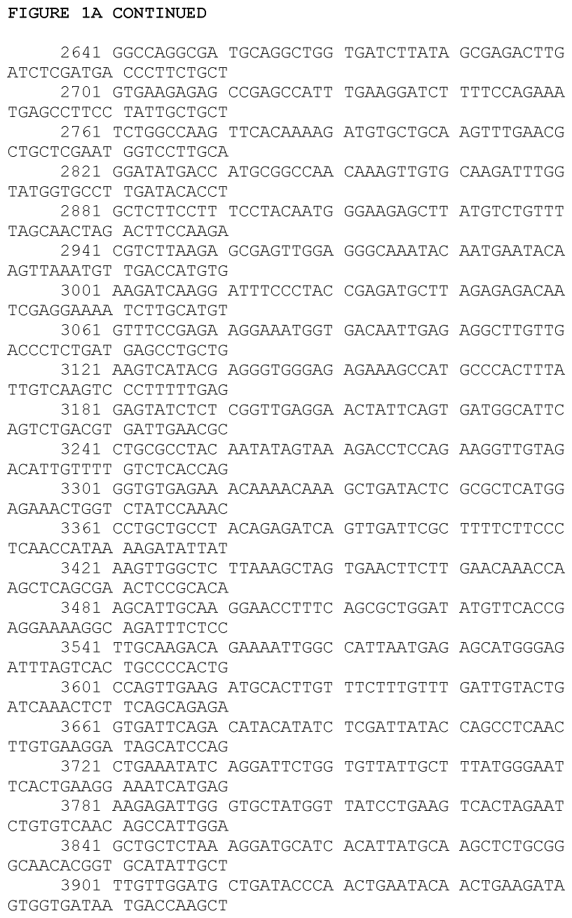

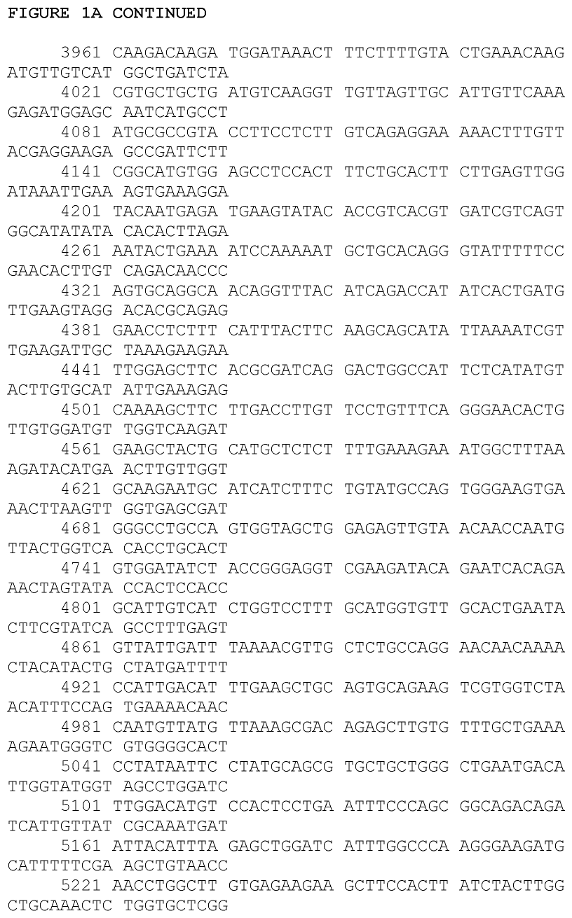

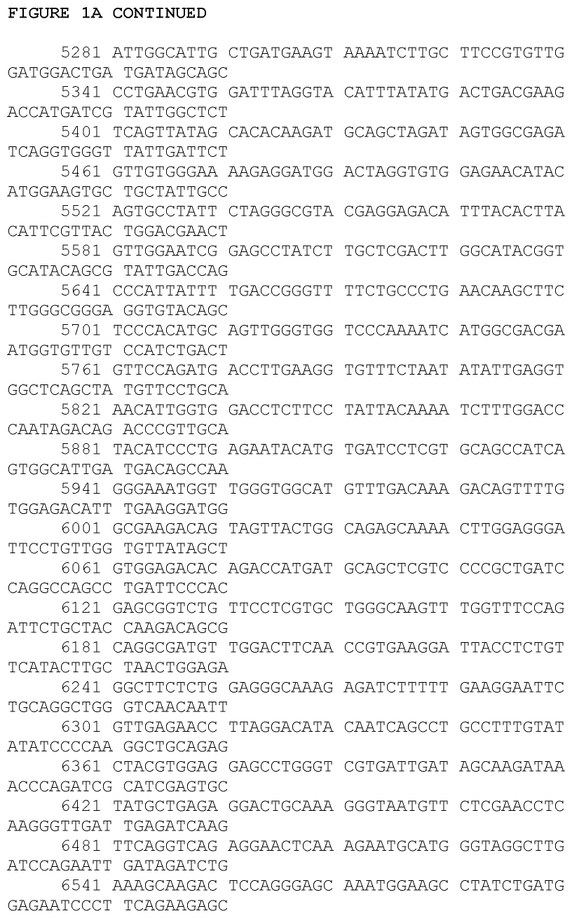

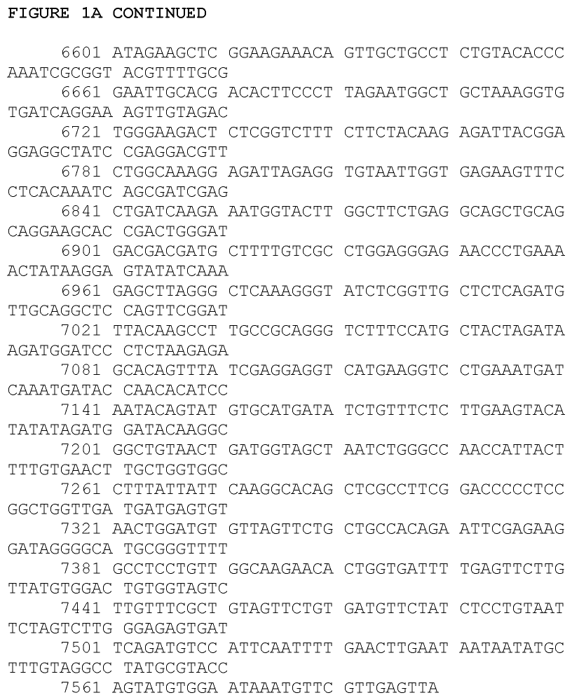

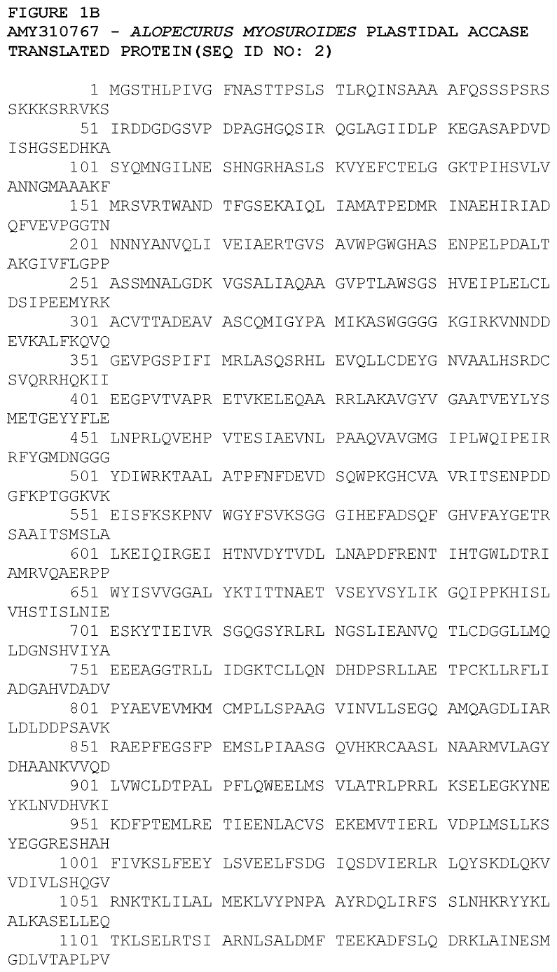

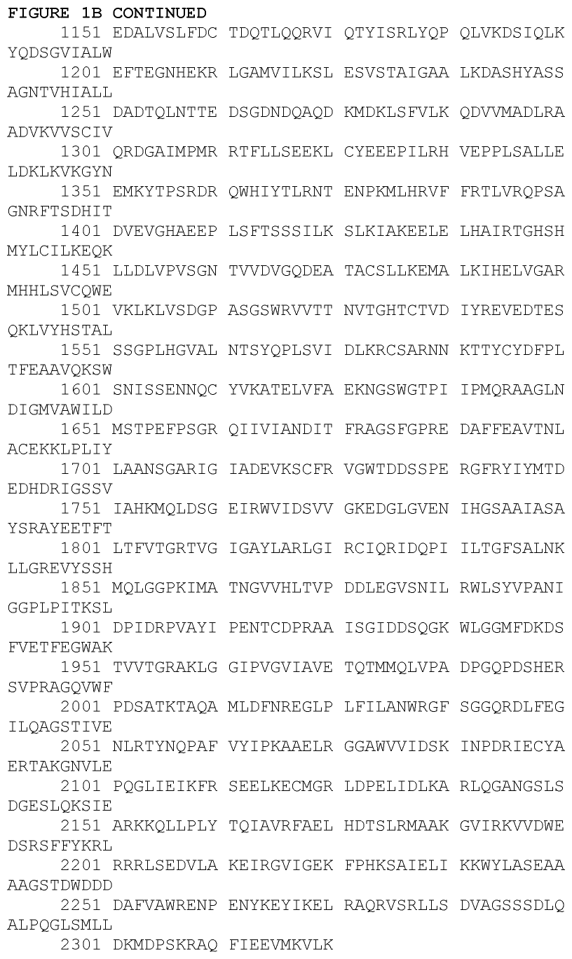

The Alopecurus myosuroides plastidal ACCase Oryza sativa ACCase gene product has the following sequence (SEQ ID NO: 2; FIG. 1B)

| | |
|---|---|
| 1 | MGSTHLPIVG FNASTTPSLS TLRQINSAAA AFQSSSPSRS SKKKSRRVKS |
| 51 | IRDDGDGSVP DPAGHGQSIR QGLAGIIDLP KEGASAPDVD ISHGSEDHKA |
| 101 | SYQMNGILNE SHNGRHASLS KVYEFCTELG GKTPIHSVLV ANNGMAAAKF |
| 151 | MRSVRTWAND TFGSEKAIQL IAMATPEDMR INAEHIRIAD QFVEVPGGTN |
| 201 | NNNYANVQLI VEIAERTGVS AVWPGWGHAS ENPELPDALT AKGIVFLGPP |
| 251 | ASSMNALGDK VGSALIAQAA GVPTLAWSGS HVEIPLELCL DSIPEEMYRK |
| 301 | ACVTTADEAV ASCQMIGYPA MIKASWGGGG KGIRKVNNDD EVKALFKQVQ |
| 351 | GEVPGSPIFI MRLASQSRHL EVQLLCDEYG NVAALHSRDC SVQRRHQKII |
| 401 | EEGPVTVAPR ETVKELEQAA RRLAKAVGYV GAATVEYLYS METGEYYFLE |
| 451 | LNPRLQVEHP VTESIAEVNL PAAQVAVGMG IPLWQIPEIR RFYGMDNGGG |
| 501 | YDIWRKTAAL ATPFNFDEVD SQWPKGHCVA VRITSENPDD GFKPTGGKVK |
| 551 | EISFKSKPNV WGYFSVKSGG GIHEFADSQF GHVFAYGETR SAAITSMSLA |
| 601 | LKEIQIRGEI HTNVDYTVDL LNAPDFRENT IHTGWLDTRI AMRVQAERPP |
| 651 | WYISVVGGAL YKTITTNAET VSEYVSYLIK GQIPPKHISL VHSTISLNIE |
| 701 | ESKYTIEIVR SGQGSYRLRL NGSLIEANVQ TLCDGGLLMQ LDGNSHVIYA |
| 751 | EEEAGGTRLL IDGKTCLLQN DHDPSRLLAE TPCKLLRFLI ADGAHVDADV |
| 801 | PYAEVEVMKM CMPLLSPAAG VINVLLSEGQ AMQAGDLIAR LDLDDPSAVK |
| 851 | RAEPFEGSFP EMSLPIAASG QVHKRCAASL NAARMVLAGY DHAANKVVQD |
| 901 | LVWCLDTPAL PFLQWEELMS VLATRLPRRL KSELEGKYNE YKLNVDHVKI |
| 951 | KDFPTEMLRE TIEENLACVS EKEMVTIERL VDPLMSLLKS YEGGRESHAH |
| 1001 | FIVKSLFEEY LSVEELFSDG IQSDVIERLR LQYSKDLQKV VDIVLSHQGV |
| 1051 | RNKTKLILAL MEKLVYPNPA AYRDQLIRFS SLNHKRYYKL ALKASELLEQ |
| 1101 | TKLSELRTSI ARNLSALDMF TEEKADFSLQ DRKLAINESM GDLVTAPLPV |
| 1151 | EDALVSLFDC TDQTLQQRVI QTYISRLYQP QLVKDSIQLK YQDSGVIALW |
| 1201 | EFTEGNHEKR LGAMVILKSL ESVSTAIGAA LKDASHYASS AGNTVHIALL |
| 1251 | DADTQLNTTE DSGDNDQAQD KMDKLSFVLK QDVVMADLRA ADVKVVSCIV |
| 1301 | QRDGAIMPMR RTFLLSEEKL CYEEEPILRH VEPPLSALLE LDKLKVKGYN |
| 1351 | EMKYTPSRDR QWHIYTLRNT ENPKMLHRVF FRTLVRQPSA GNRFTSDHIT |
| 1401 | DVEVGHAEEP LSFTSSSILK SLKIAKEELE LHAIRTGHSH MYLCILKEQK |
| 1451 | LLDLVPVSGN TVVDVGQDEA TACSLLKEMA LKIHELVGAR MHHLSVCQWE |
| 1501 | VKLKLVSDGP ASGSWRVVTT NVTGHTCTVD IYREVEDTES QKLVYHSTAL |
| 1551 | SSGPLHGVAL NTSYQPLSVI DLKRCSARNN KTTYCYDFPL TFEAAVQKSW |
| 1601 | SNISSENNQC YVKATELVFA EKNGSWGTPI IPMQRAAGLN DIGMVAWILD |
| 1651 | MSTPEFPSGR QIIVIANDIT FRAGSFGPRE DAFFEAVTNL ACEKKLPLIY |
| 1701 | LAANSGARIG IADEVKSCFR VGWTDDSSPE RGFRYIYMTD EDHDRIGSSV |
| 1751 | IAHKMQLDSG EIRWVIDSVV GKEDGLGVEN IHGSAAIASA YSRAYEETFT |
| 1801 | LTFVTGRTVG IGAYLARLGI RCIQRIDQPI ILTGFSALNK LLGREVYSSH |
| 1851 | MQLGGPKIMA TNGVVHLTVP DDLEGVSNIL RWLSYVPANI GGPLPITKSL |
| 1901 | DPIDRPVAYI PENTCDPRAA ISGIDDSQGK WLGGMFDKDS FVETFEGWAK |
| 1951 | TVVTGRAKLG GIPVGVIAVE TQTMMQLVPA DPGQPDSHER SVPRAGQVWF |
| 2001 | PDSATKTAQA MLDFNREGLP LFILANWRGF SGGQRDLFEG ILQAGSTIVE |
| 2051 | NLRTYNQPAF VYIPKAAELR GGAWVVIDSK INPDRIECYA ERTAKGNVLE |
| 2101 | PQGLIEIKFR SEELKECMGR LDPELIDLKA RLQGANGSLS DGESLQKSIE |
| 2151 | ARKKQLLPLY TQIAVRFAEL HDTSLRMAAK GVIRKVVDWE DSRSFFYKRL |
| 2201 | RRRLSEDVLA KEIRGVIGEK FPHKSAIELI KKWYLASEAA AAGSTDWDDD |
| 2251 | DAFVAWRENP ENYKEYIKEL RAQRVSRLLS DVAGSSSDLQ ALPQGLSMLL |
| 2301 | DKMDPSKRAQ FIEEVMKVLK |

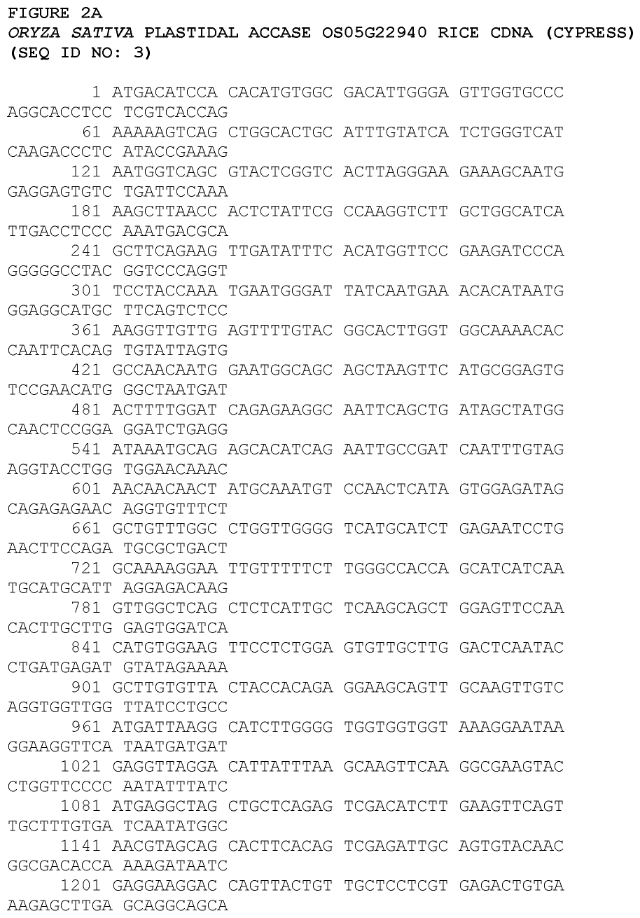

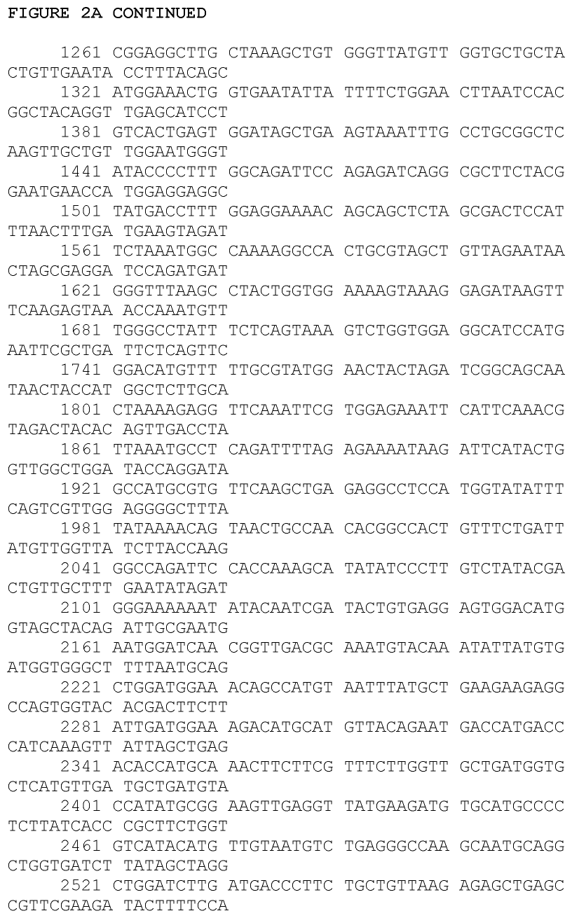

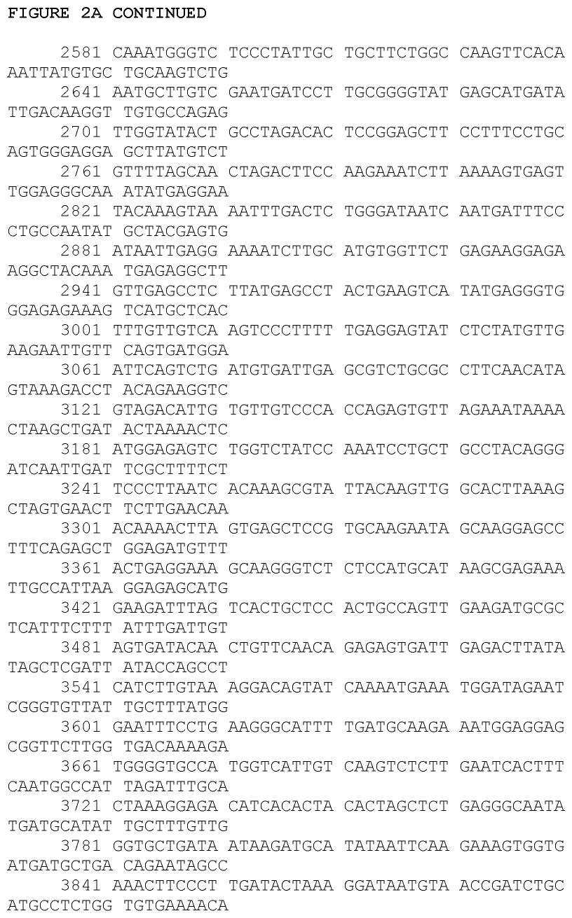

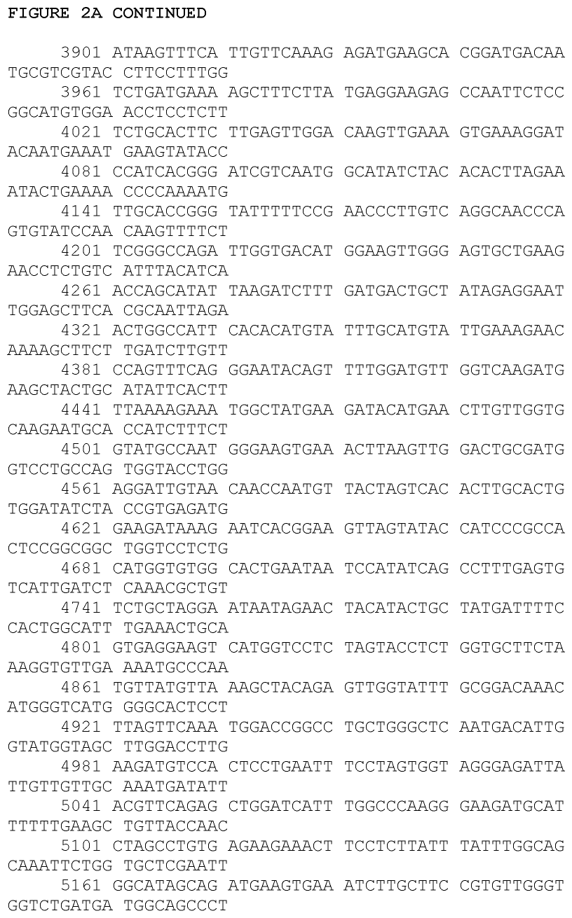

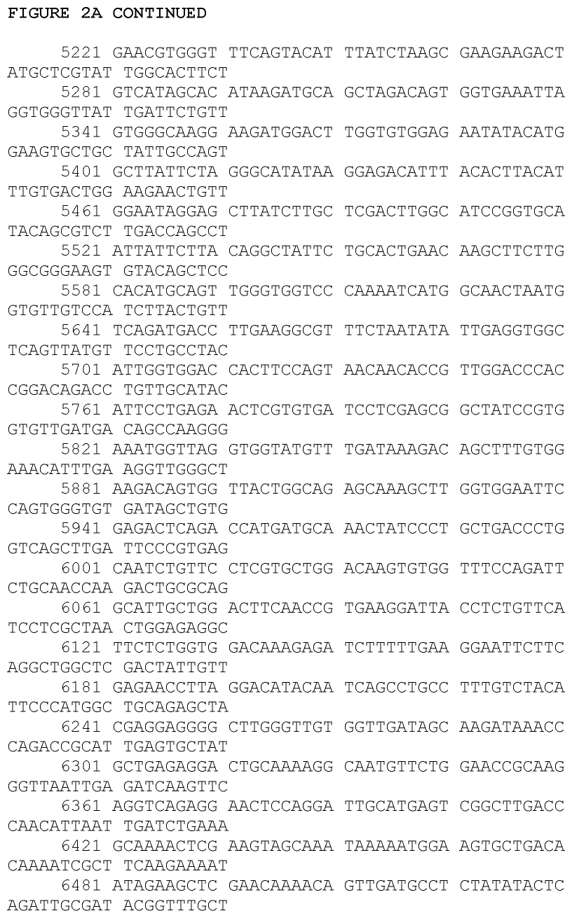

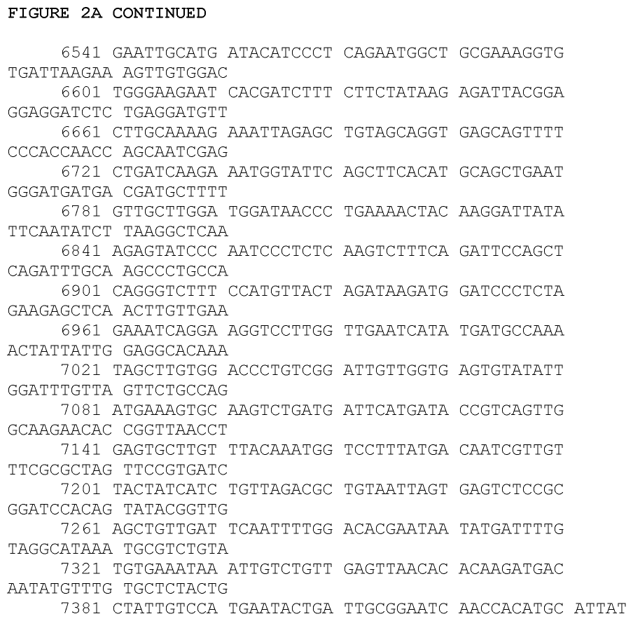

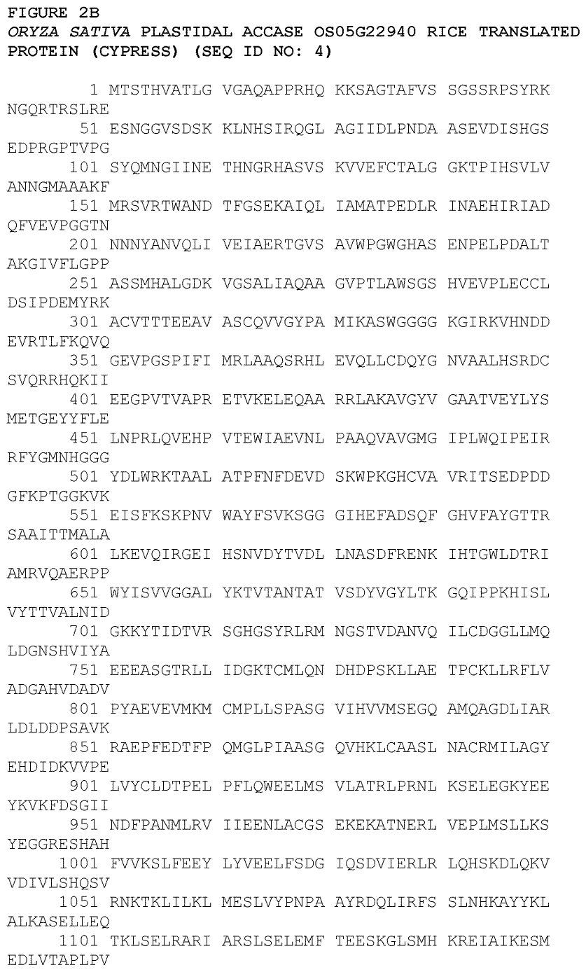

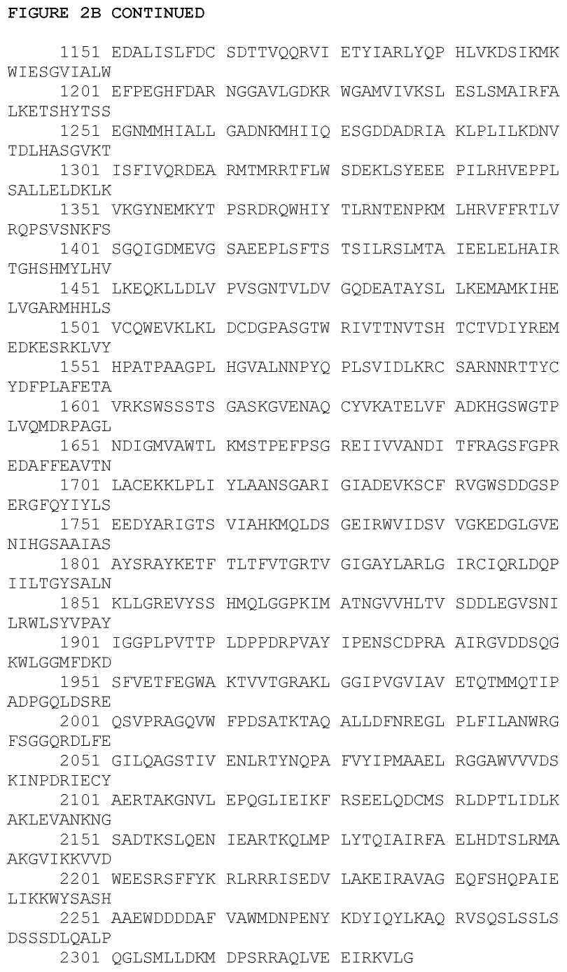

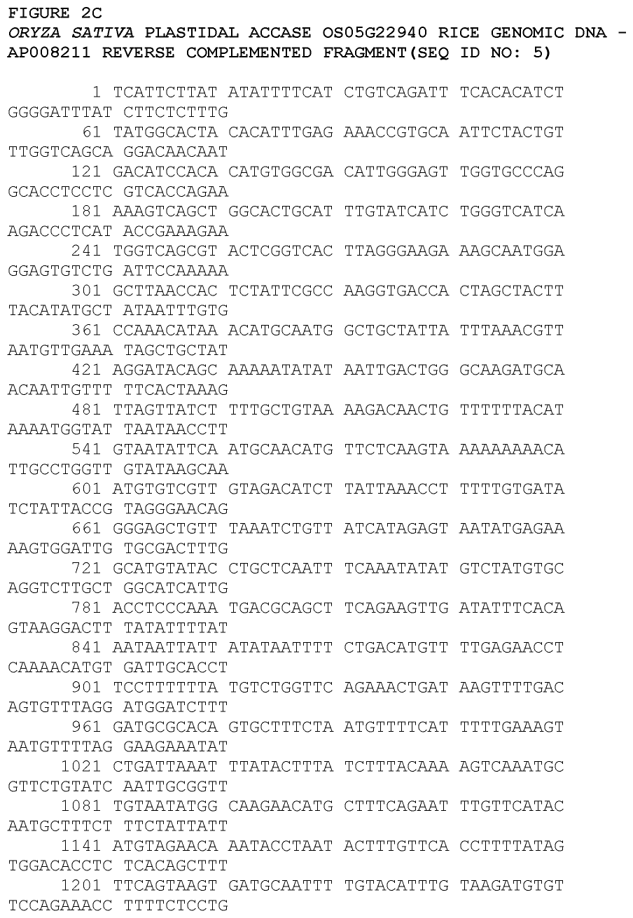

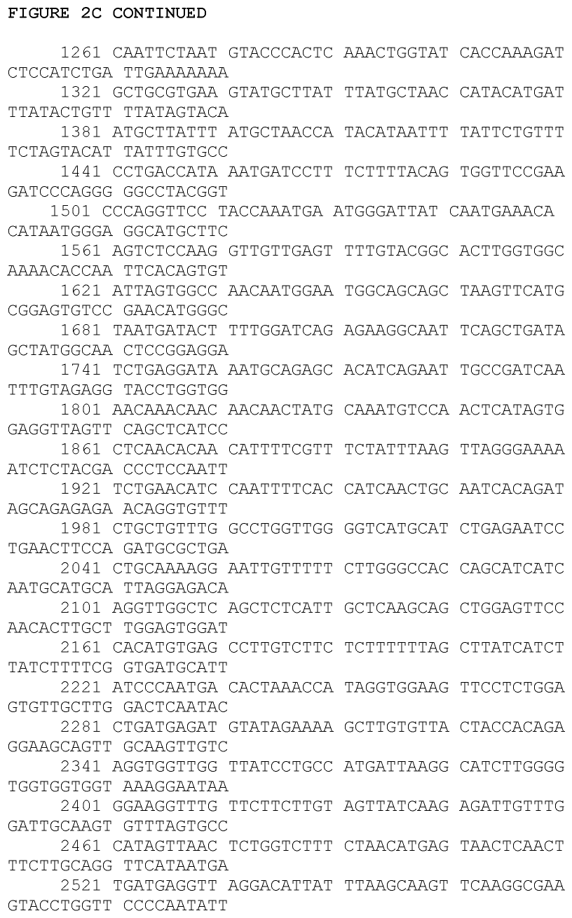

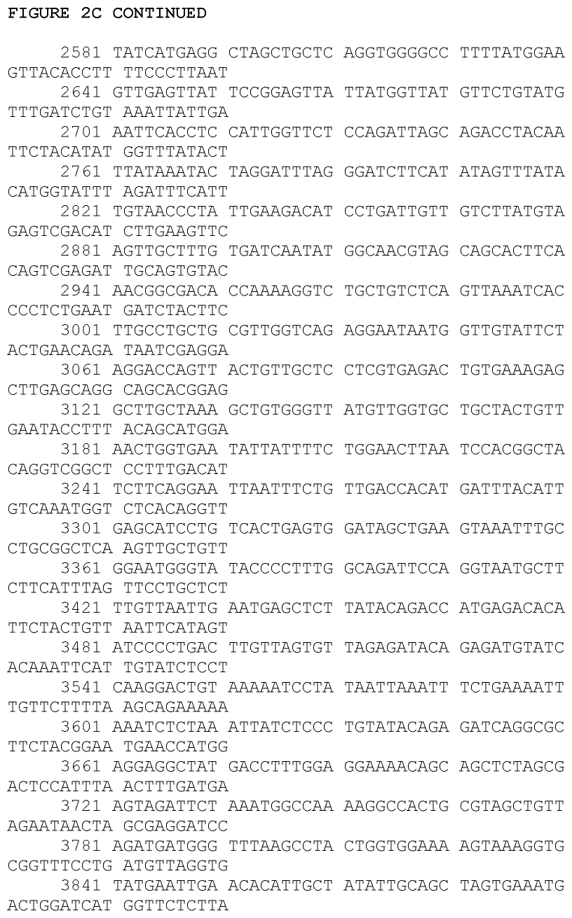

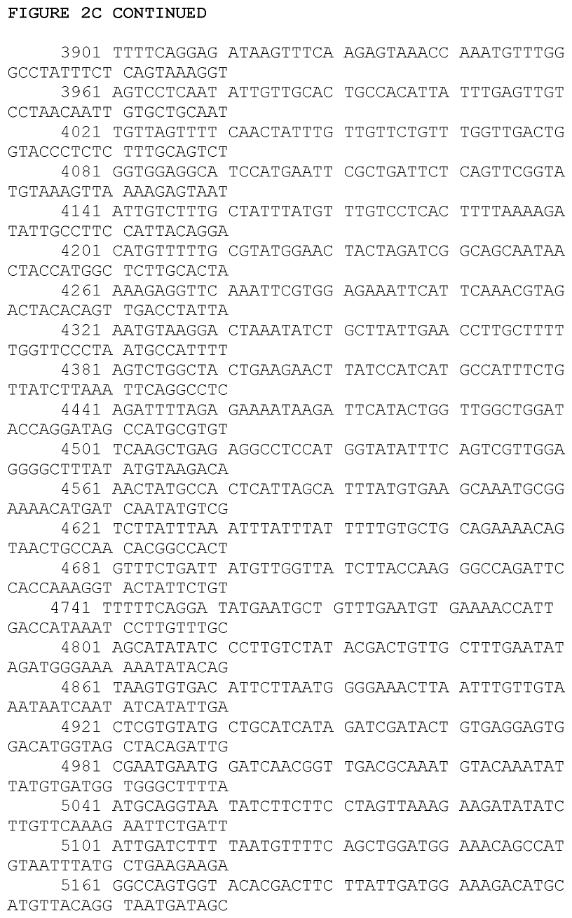

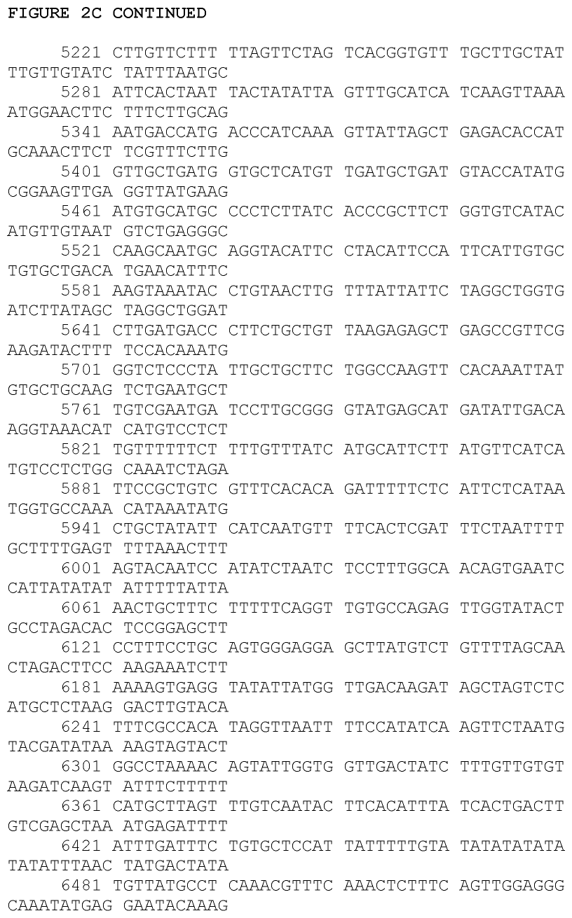

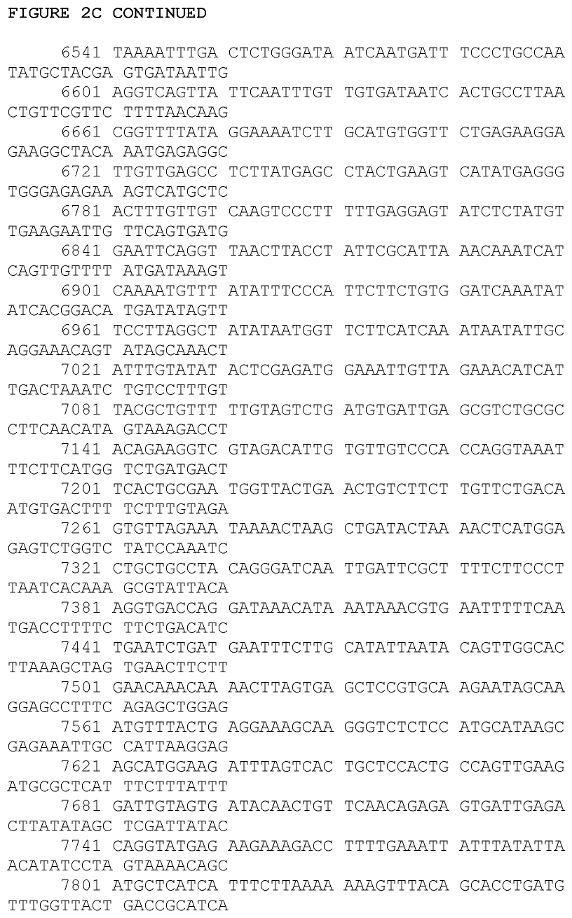

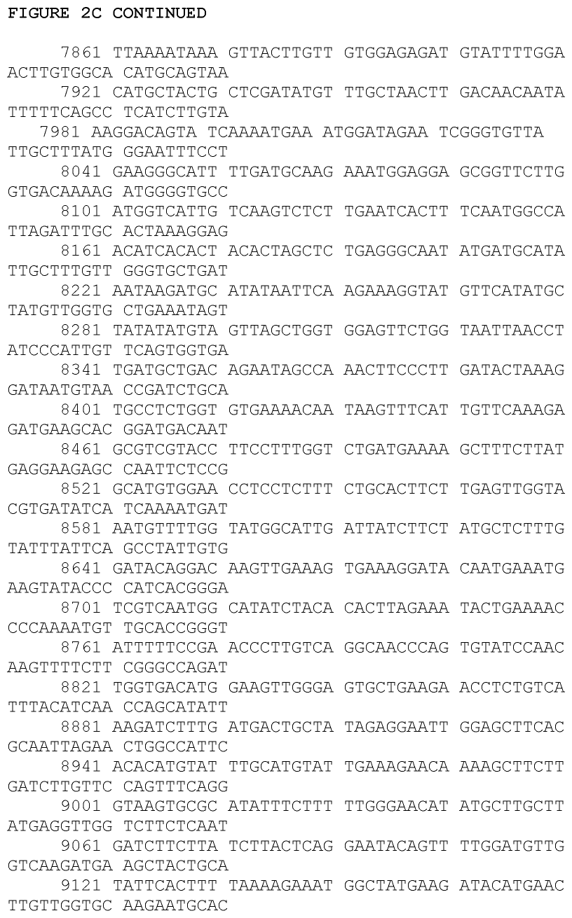

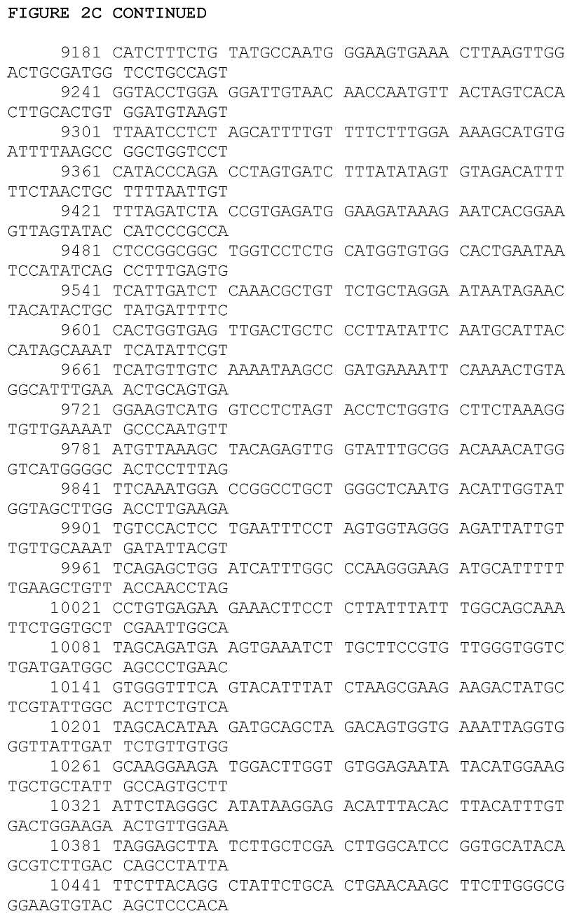

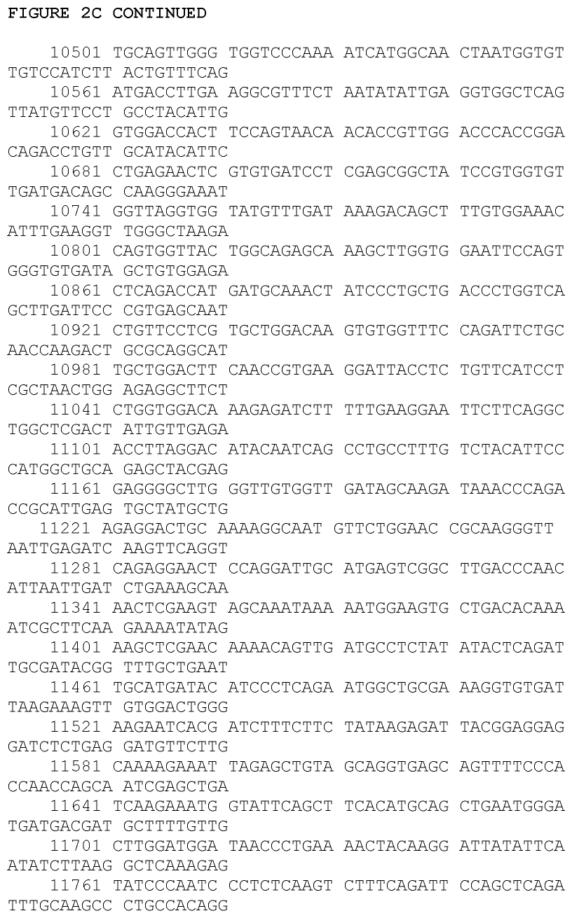

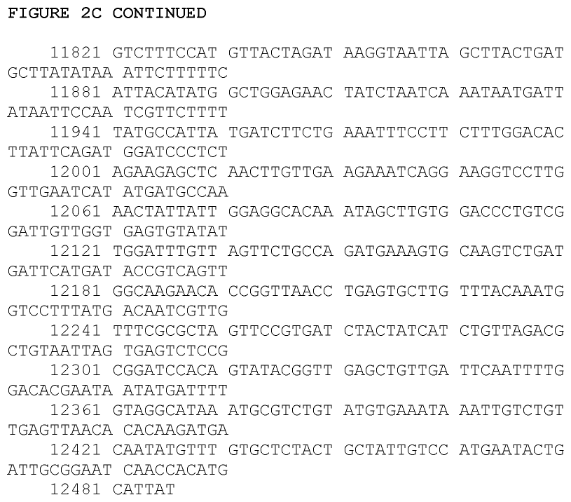

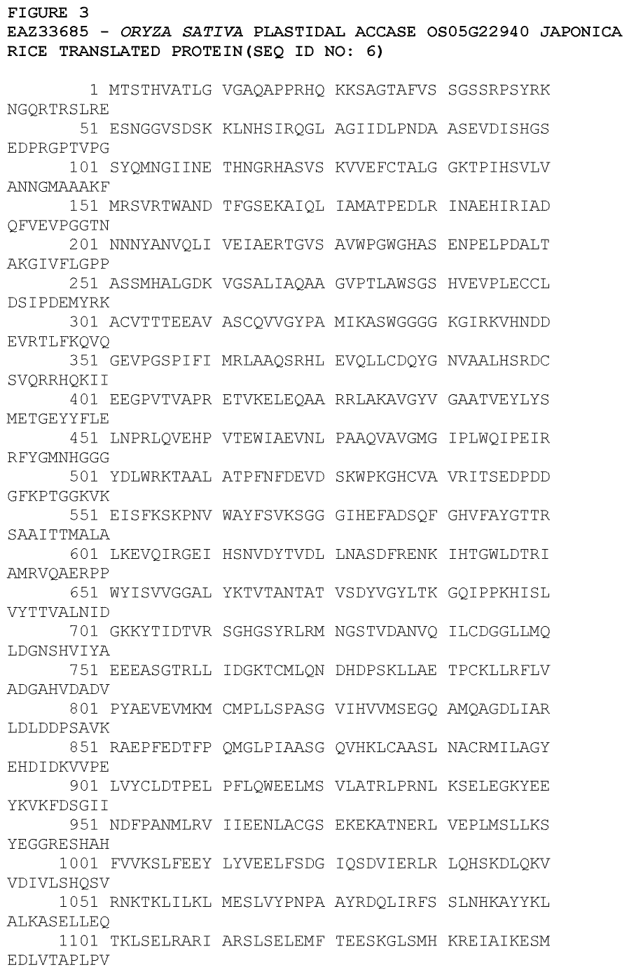

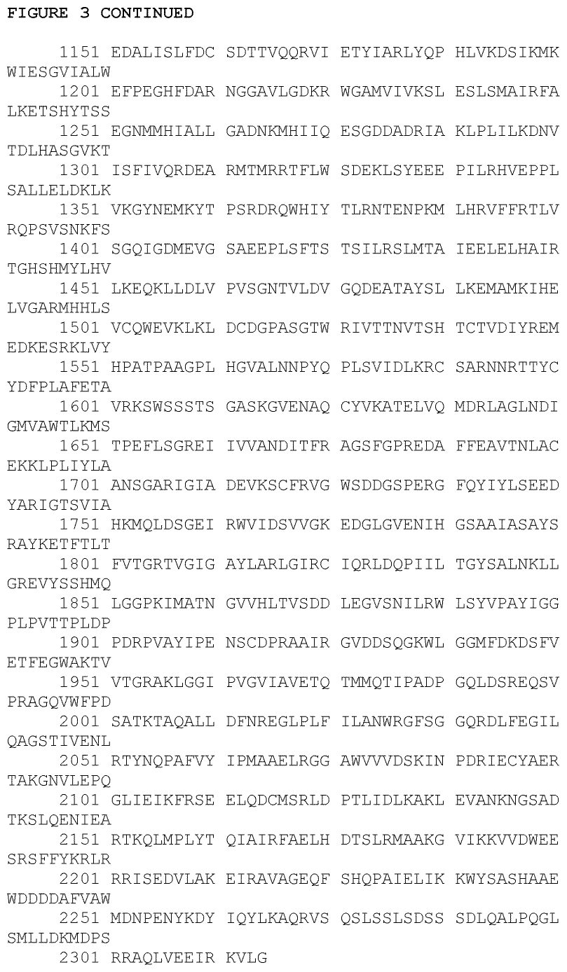

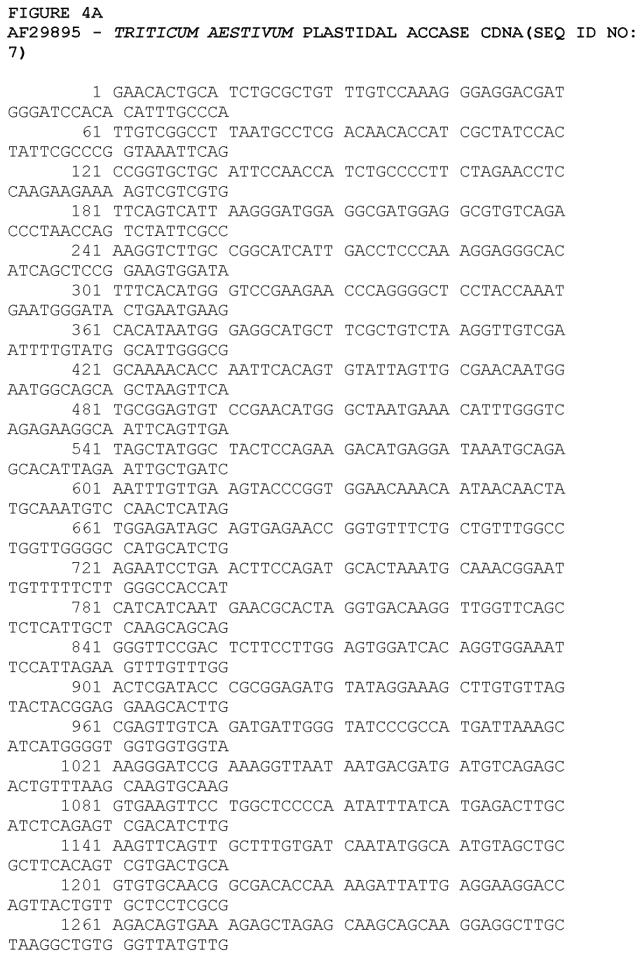

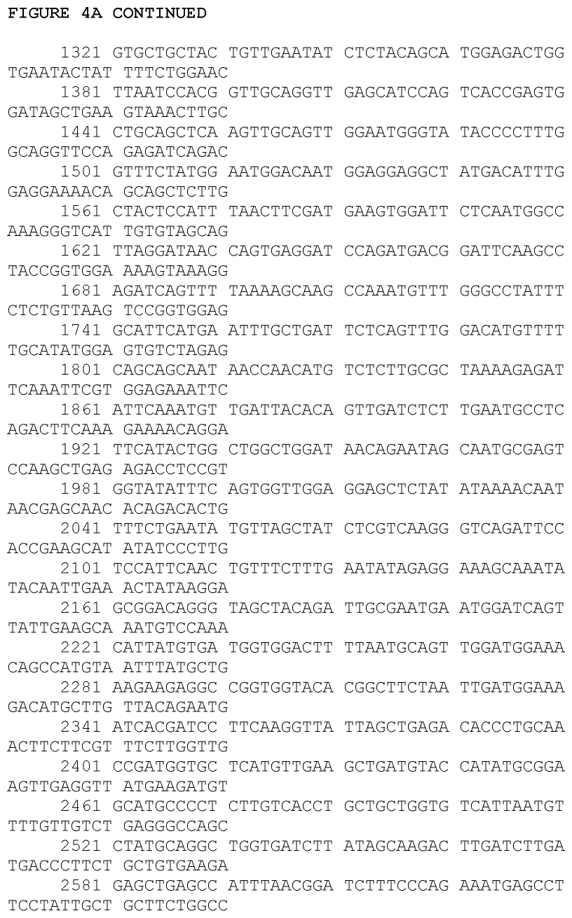

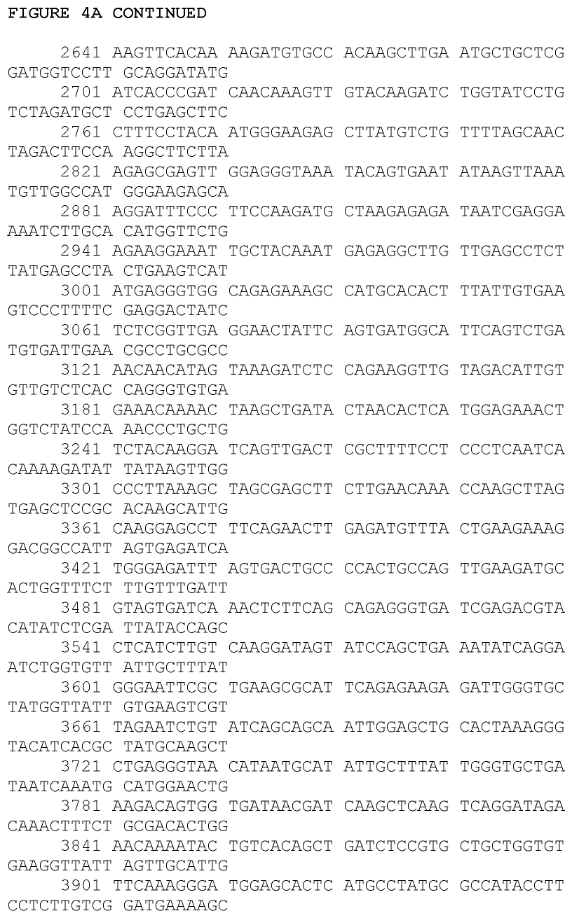

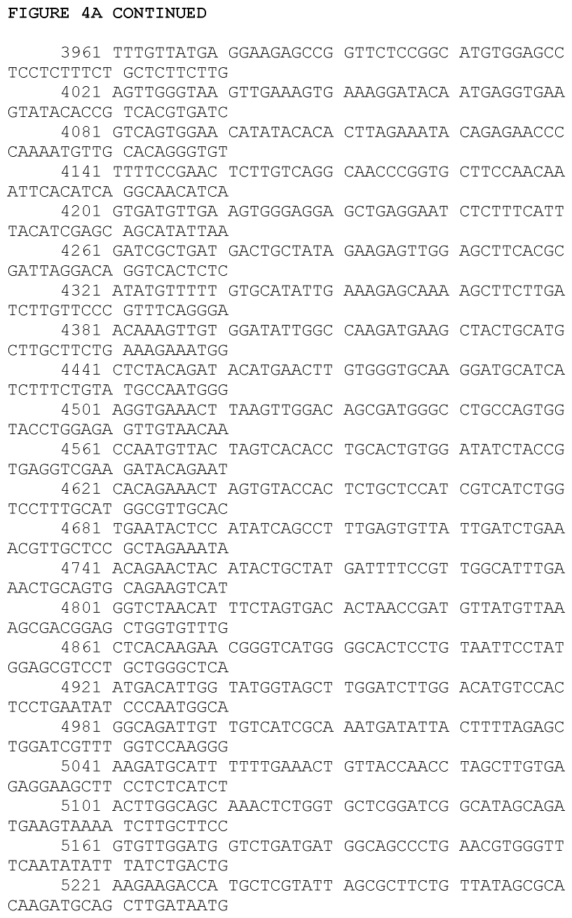

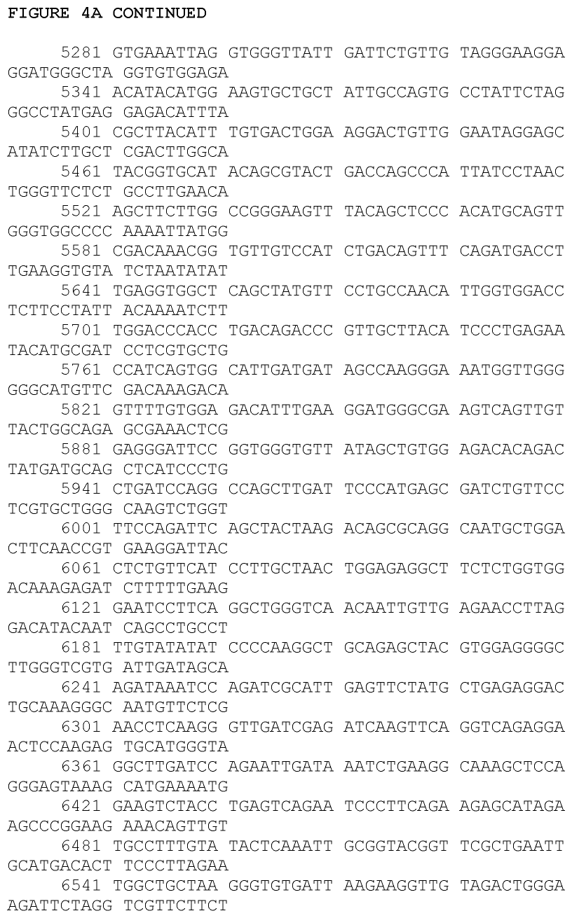

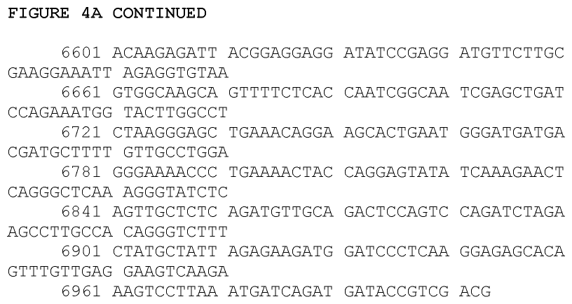

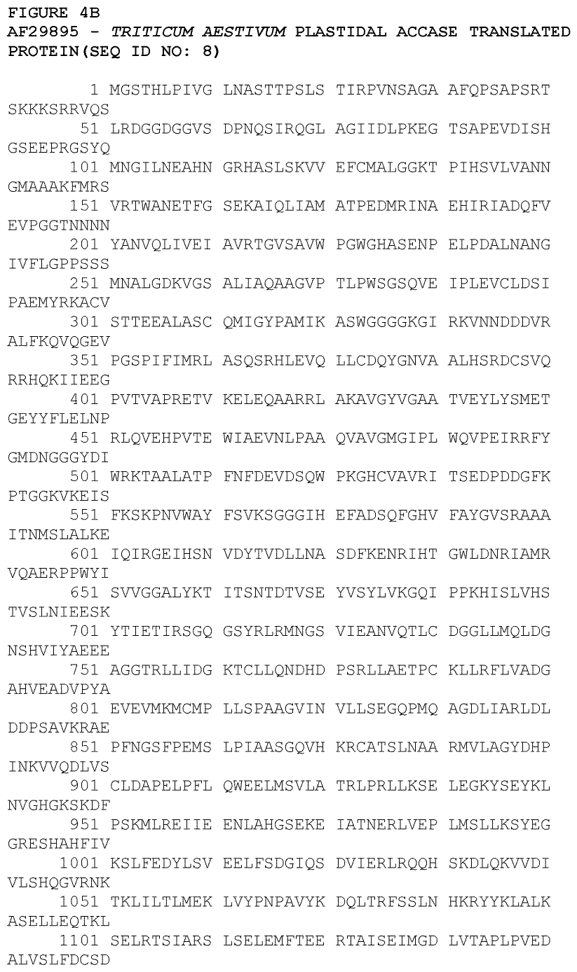

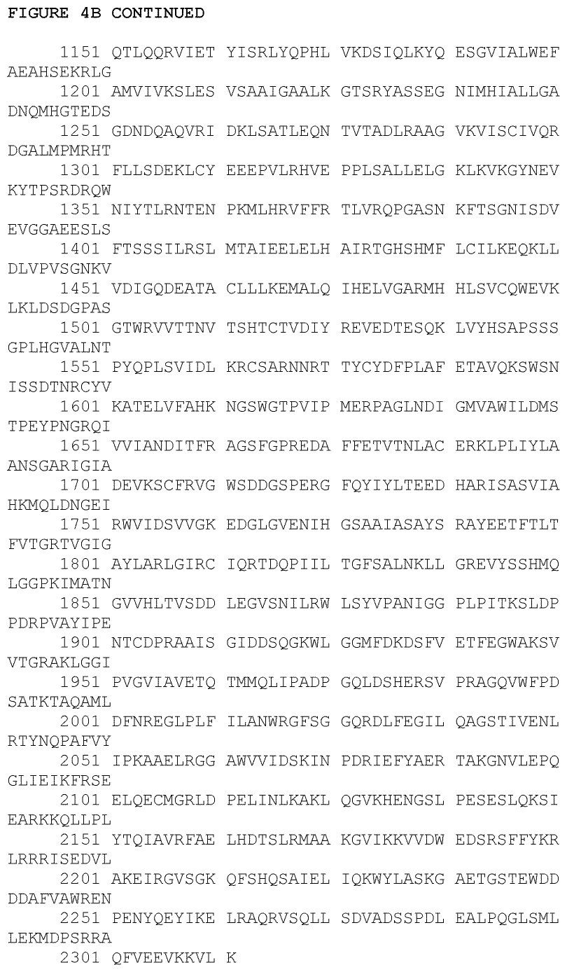

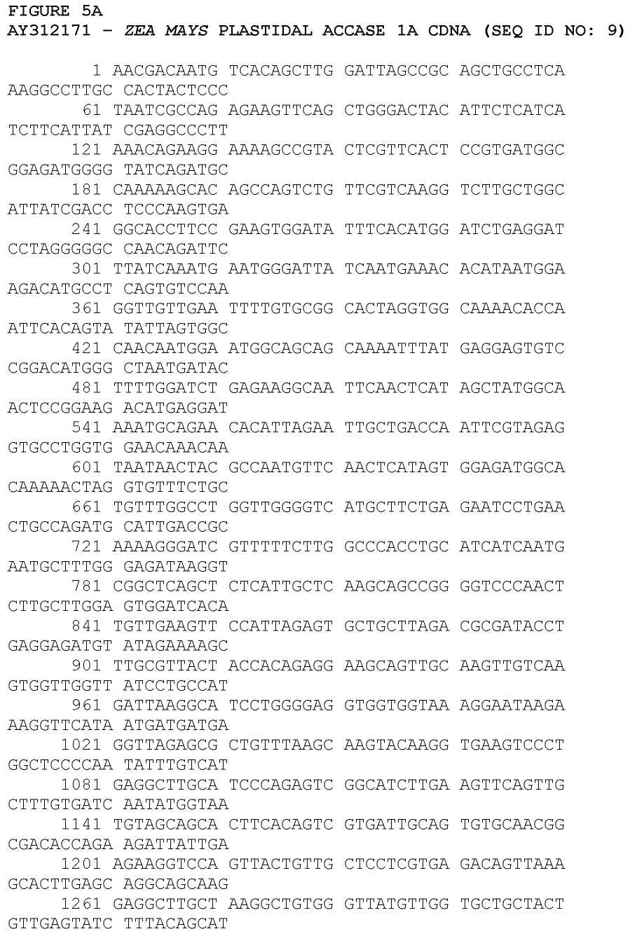

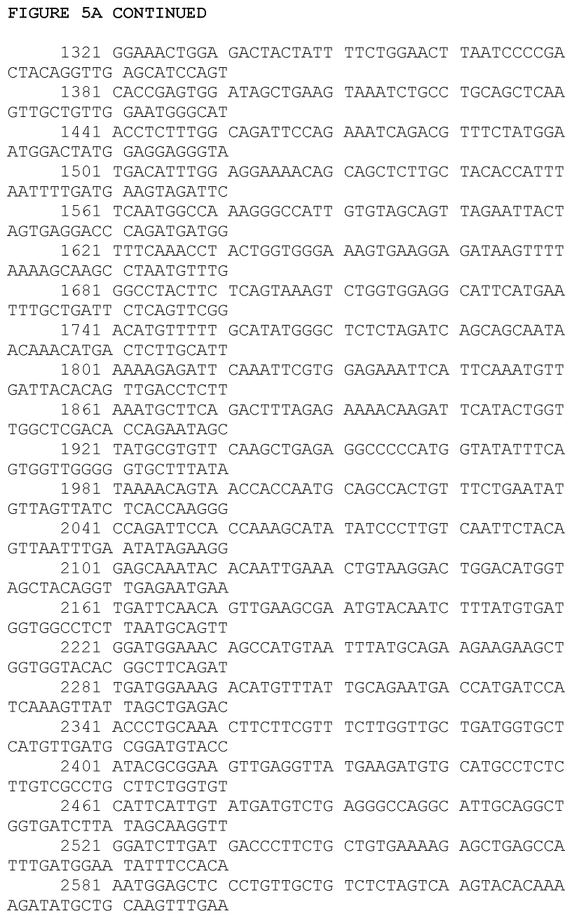

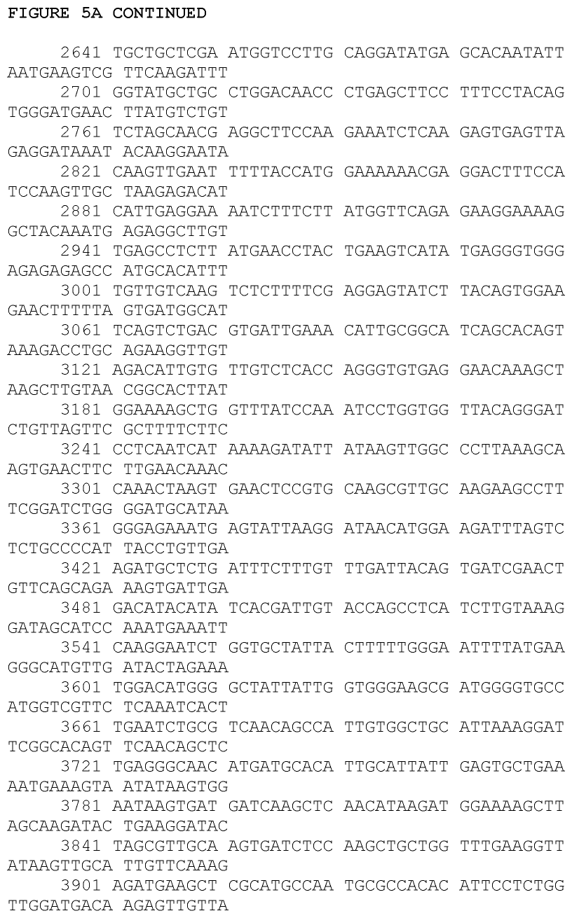

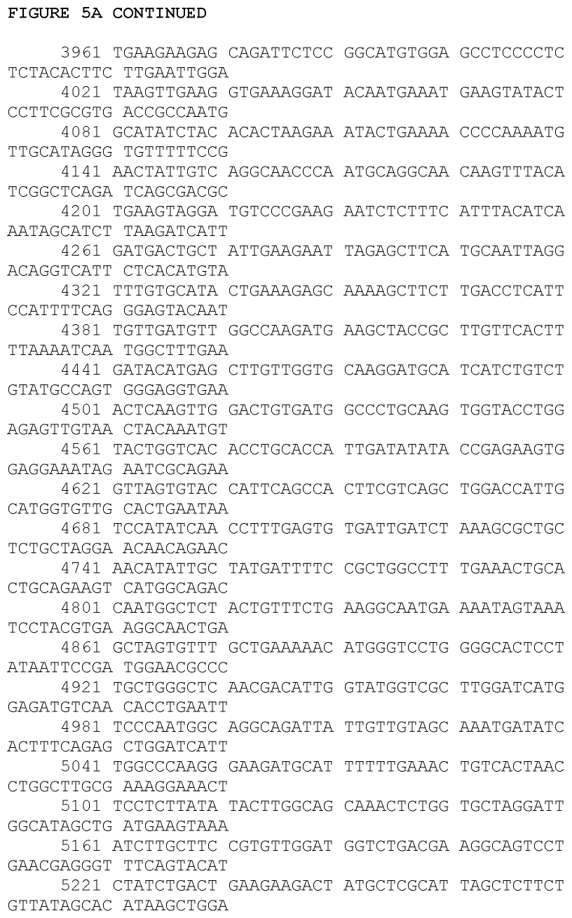

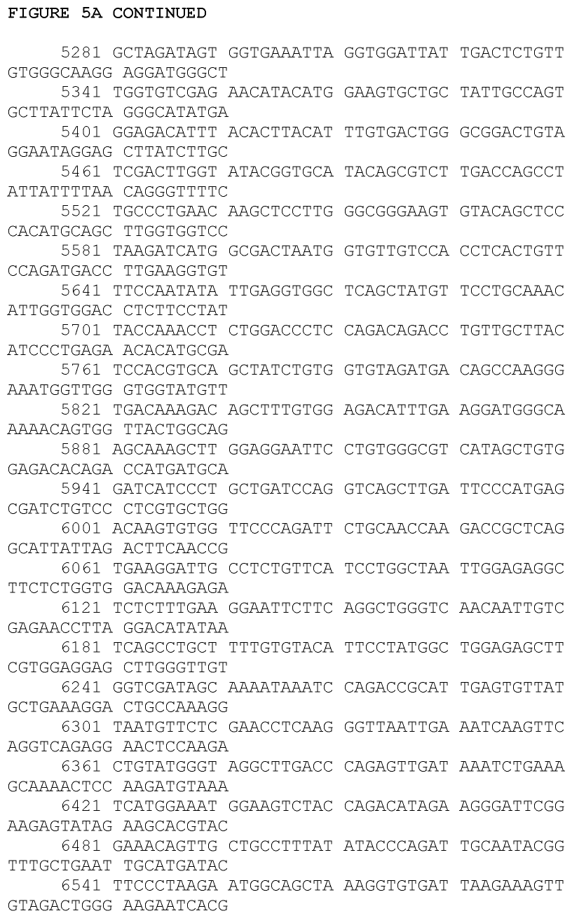

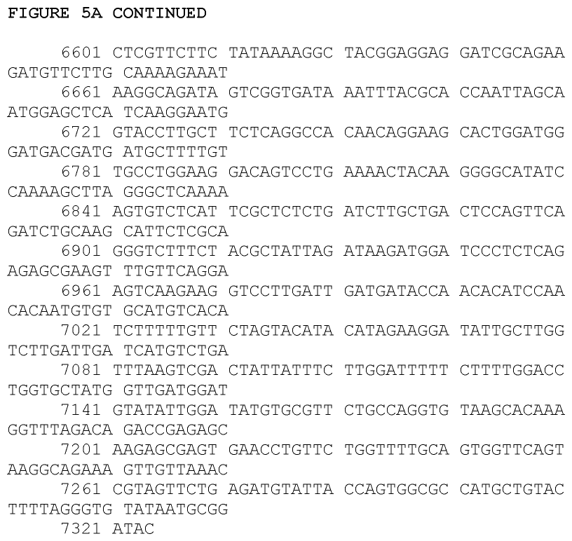

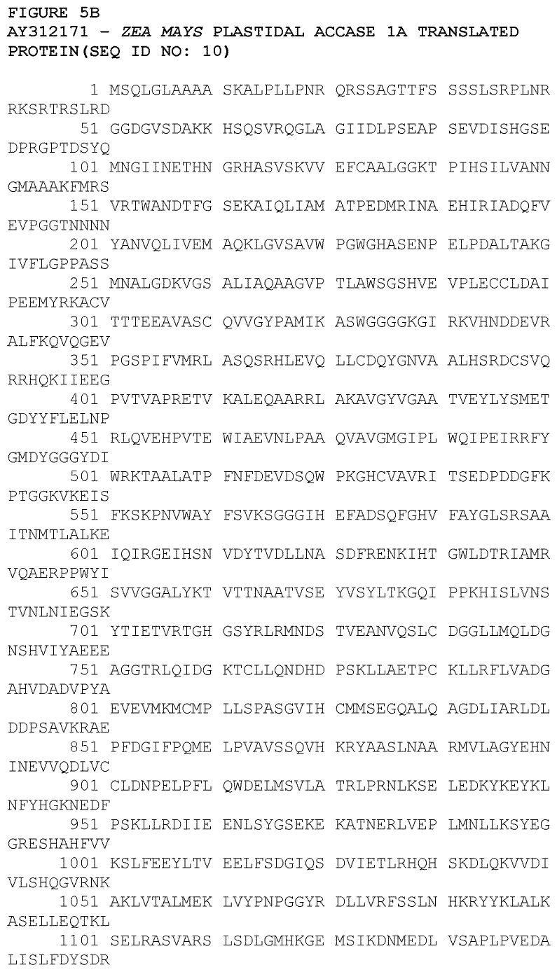

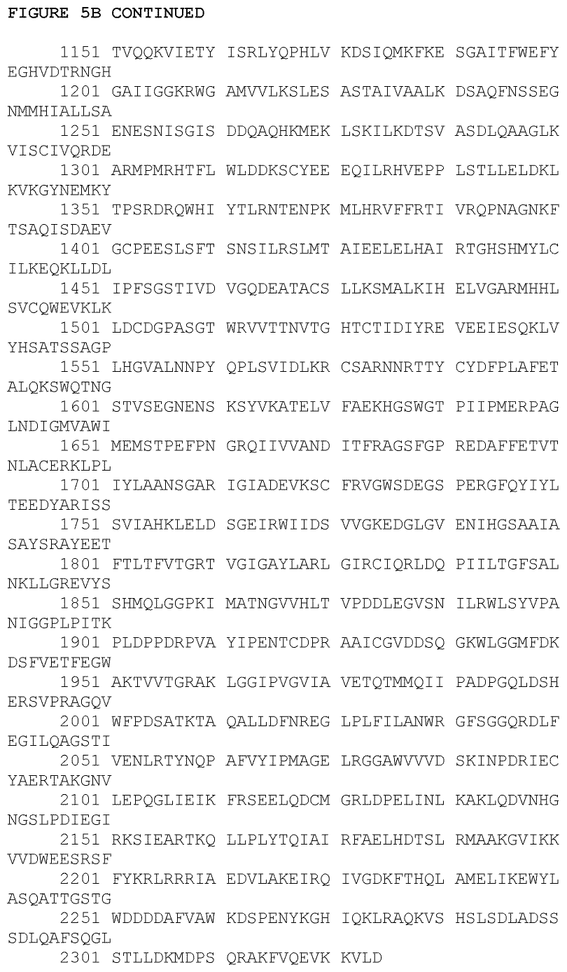

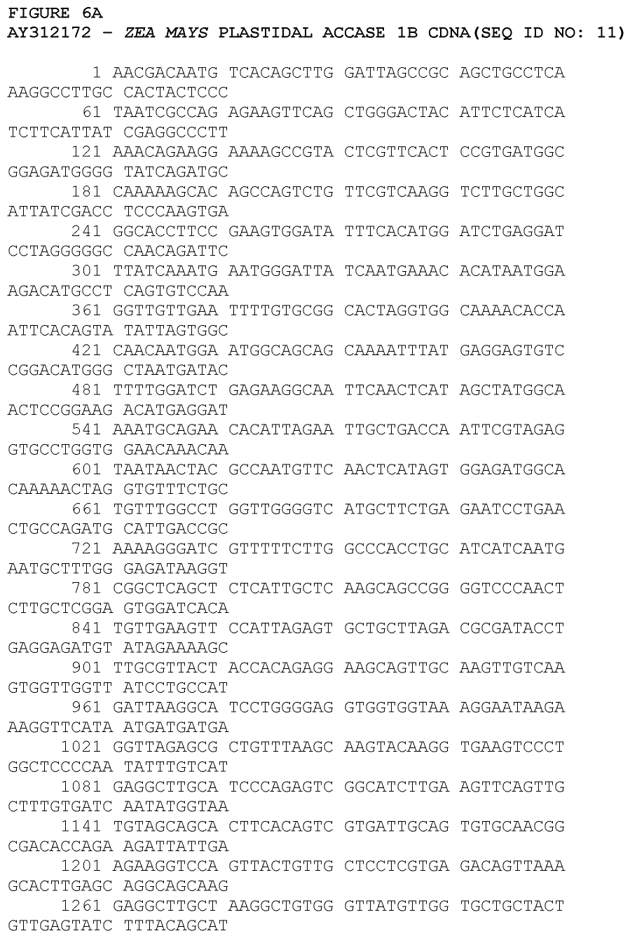

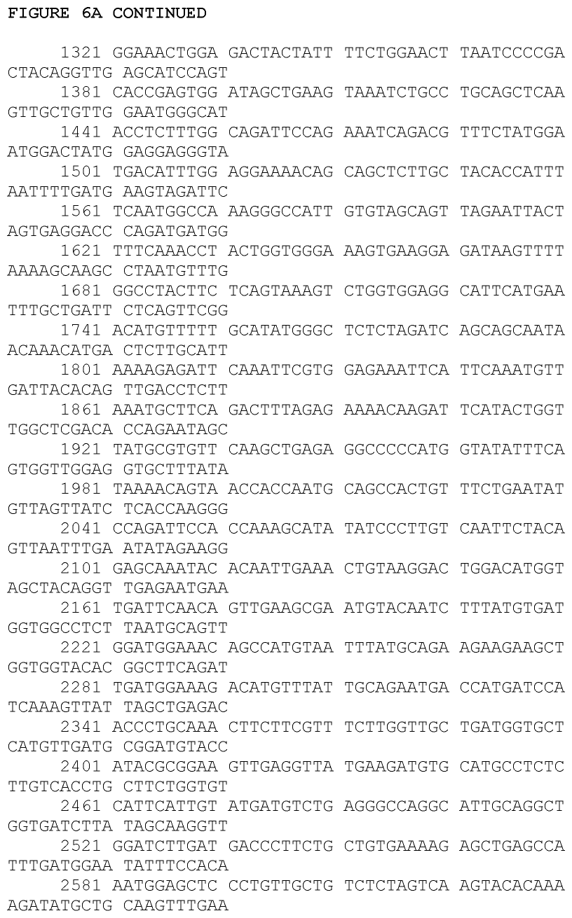

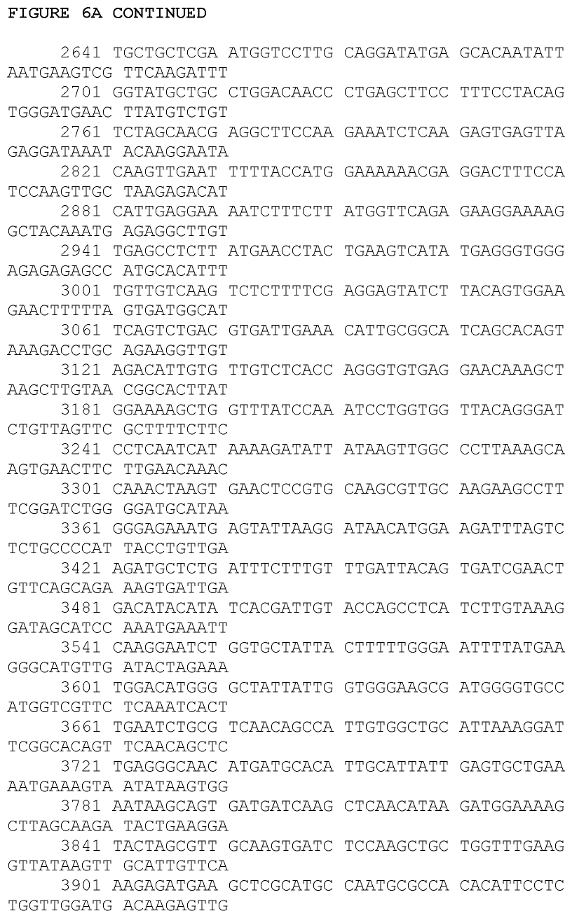

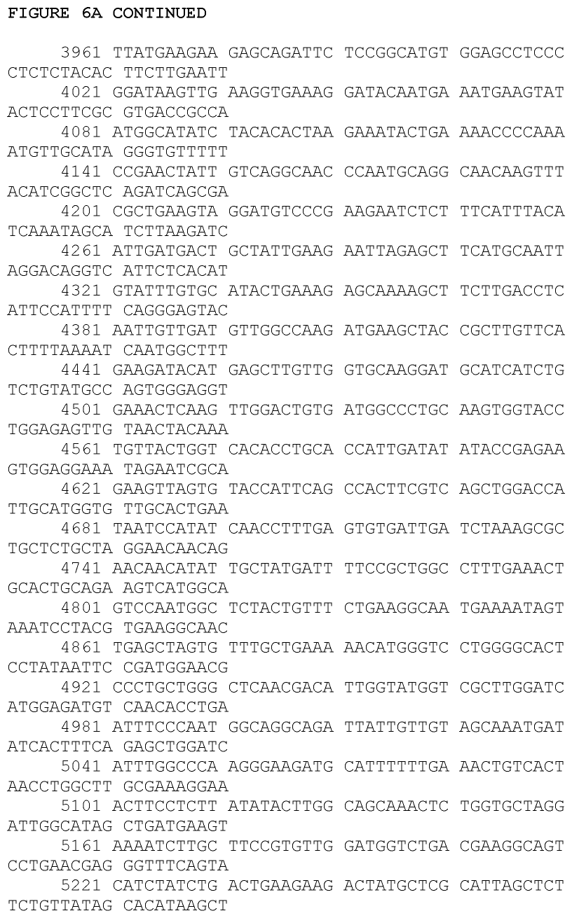

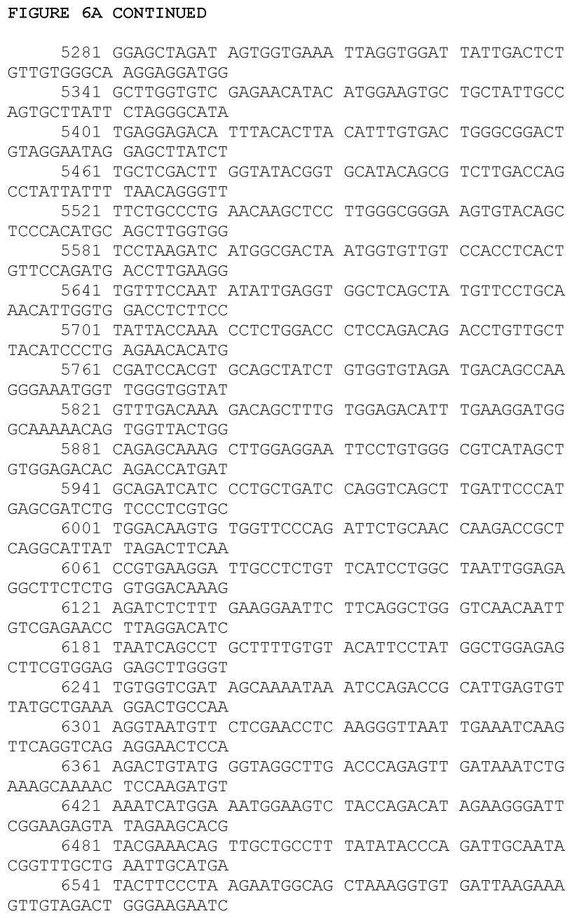

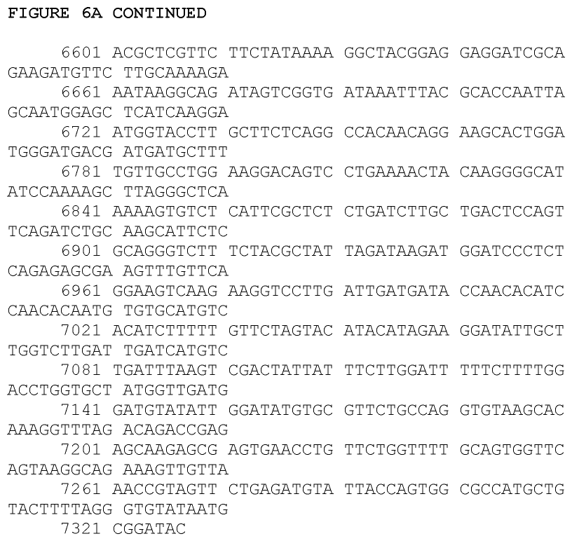

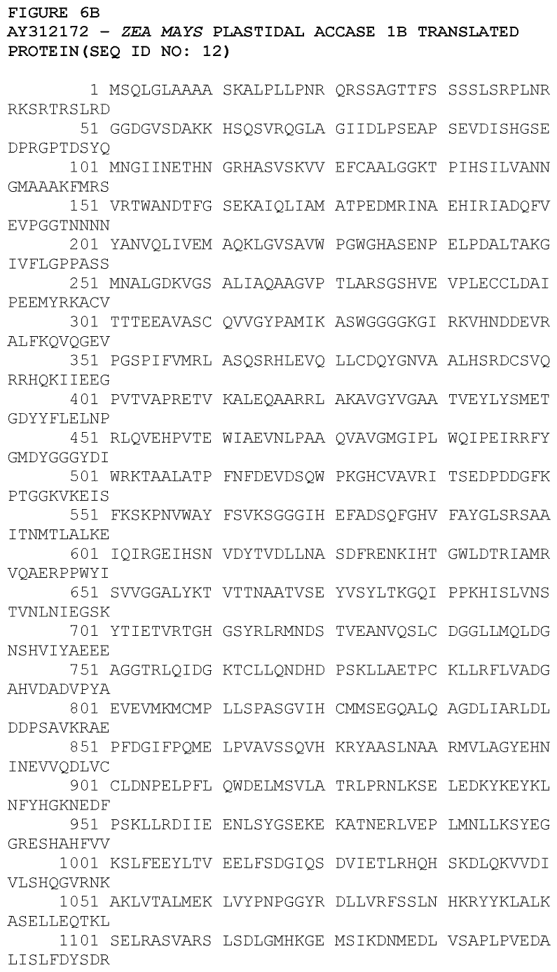

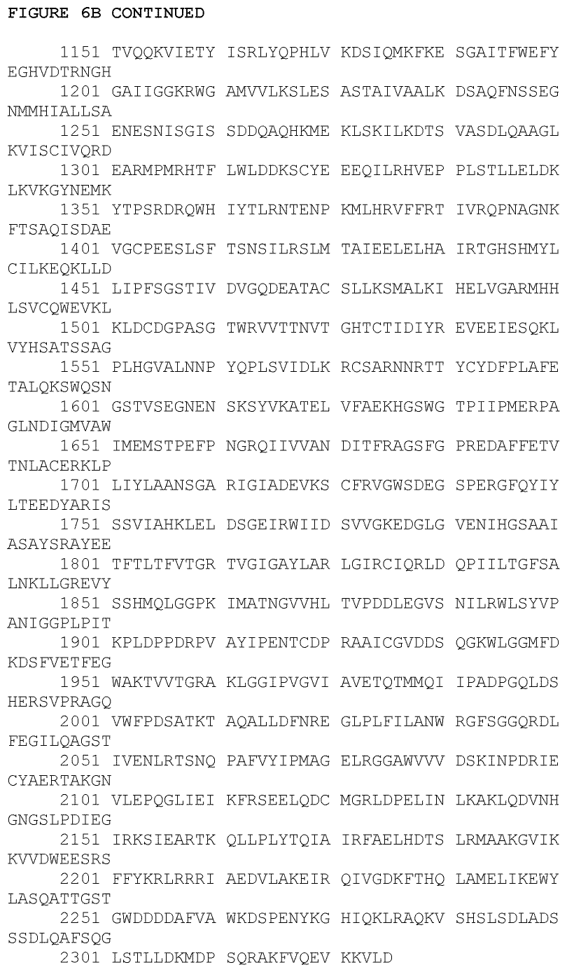

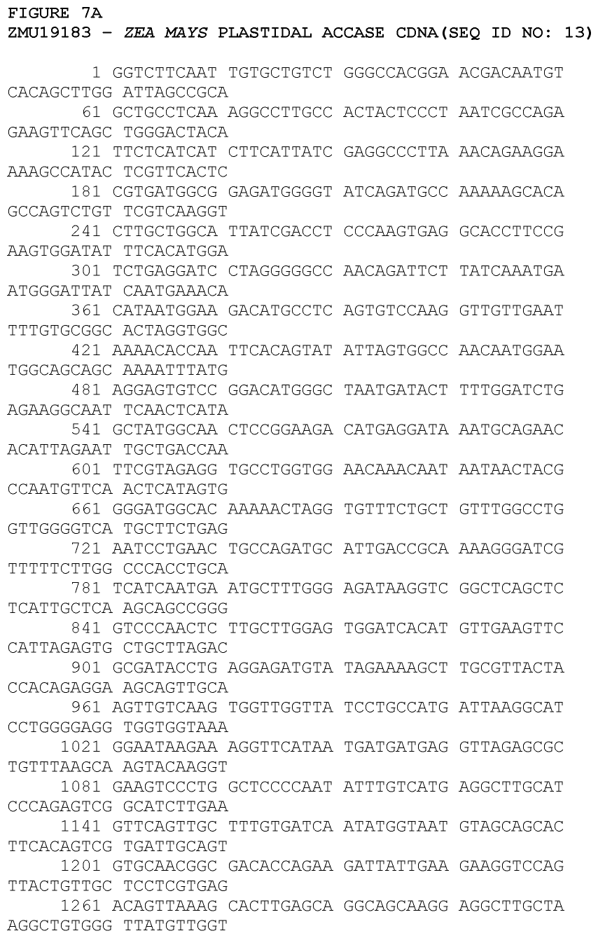

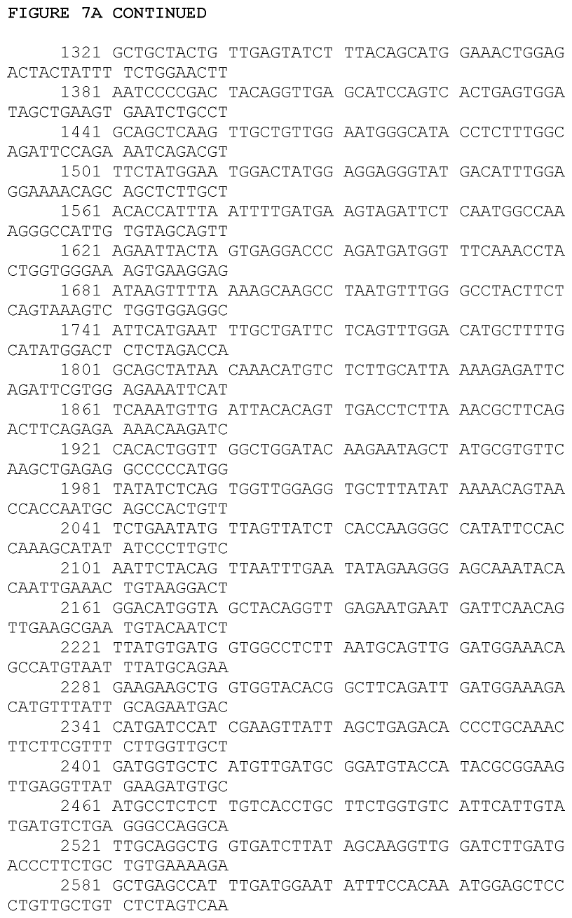

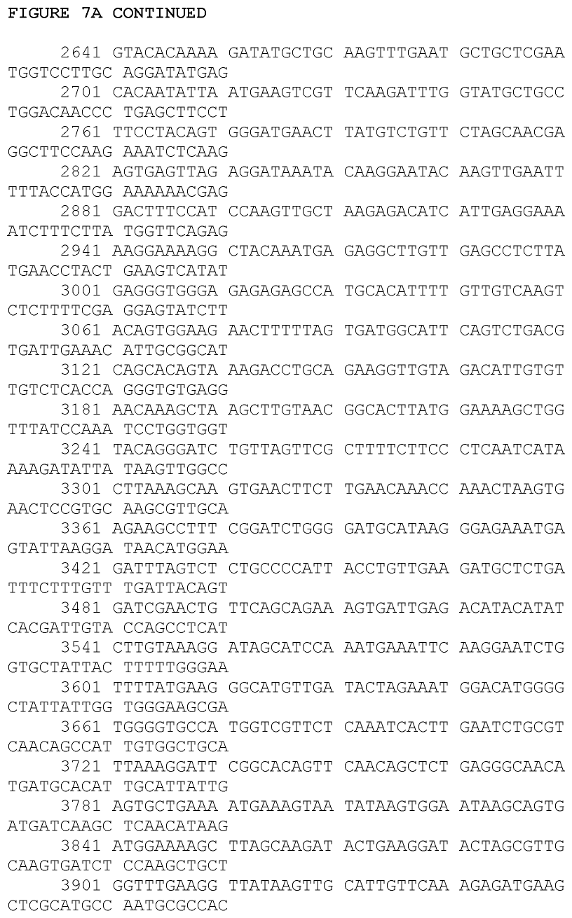

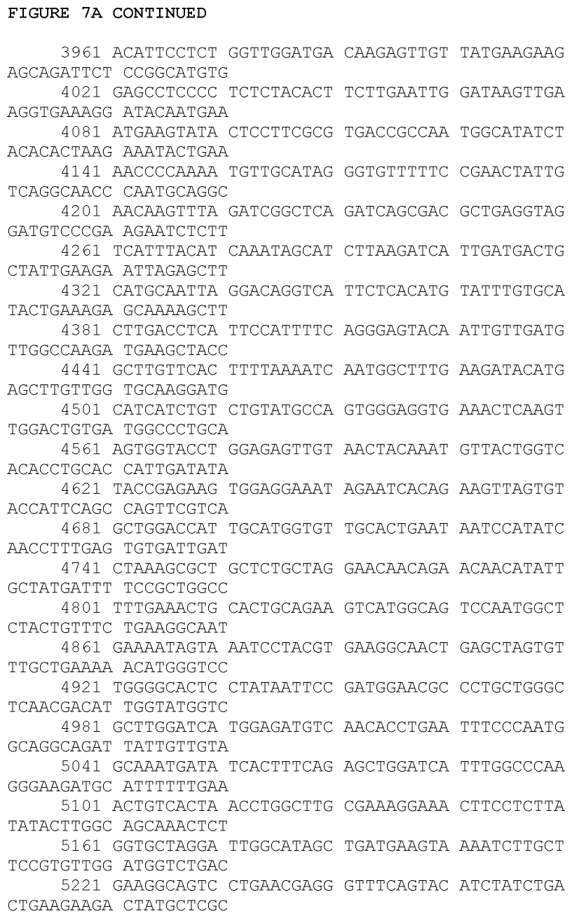

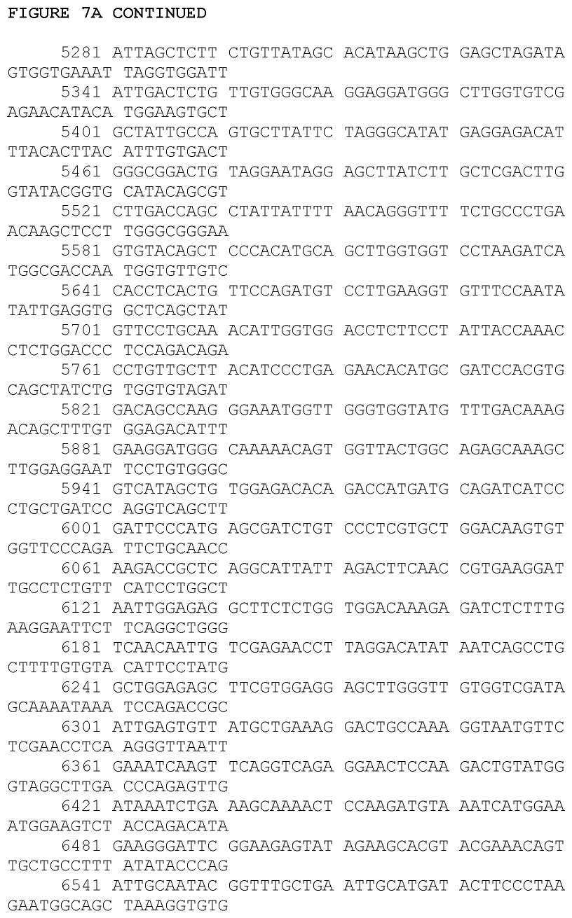

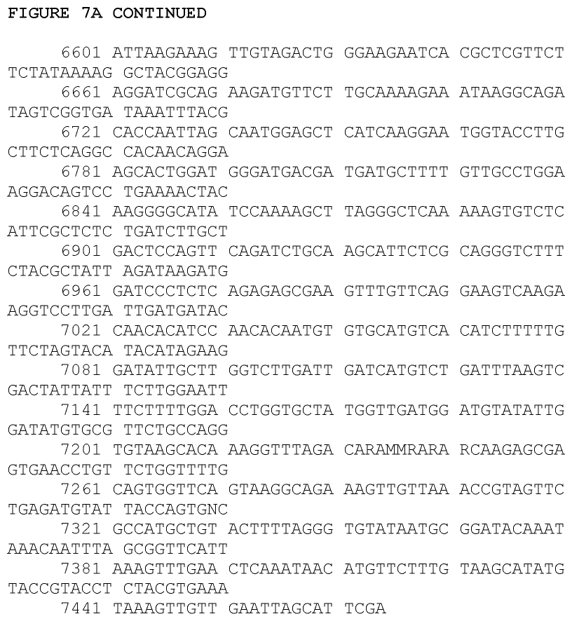

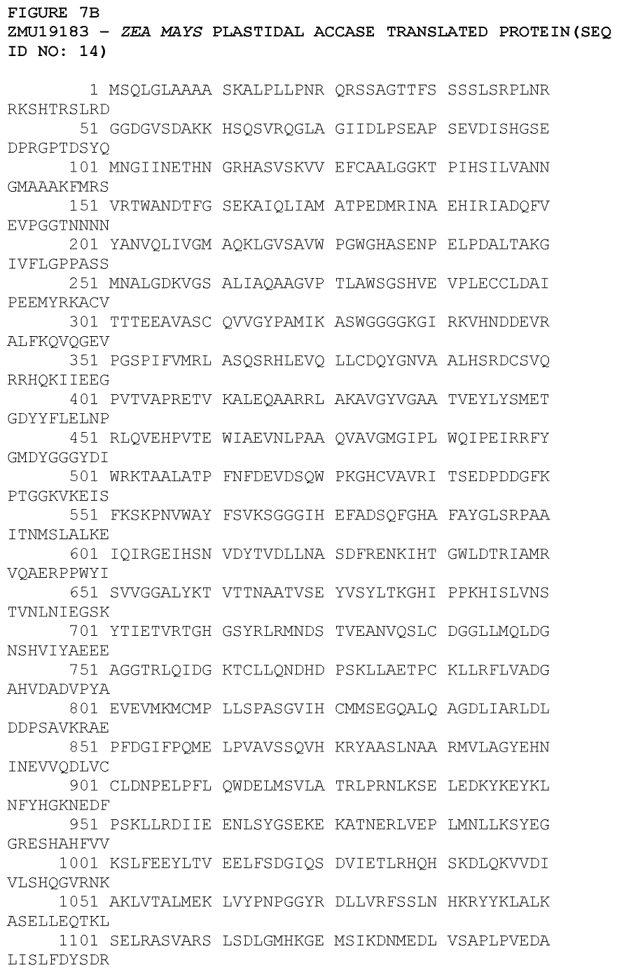

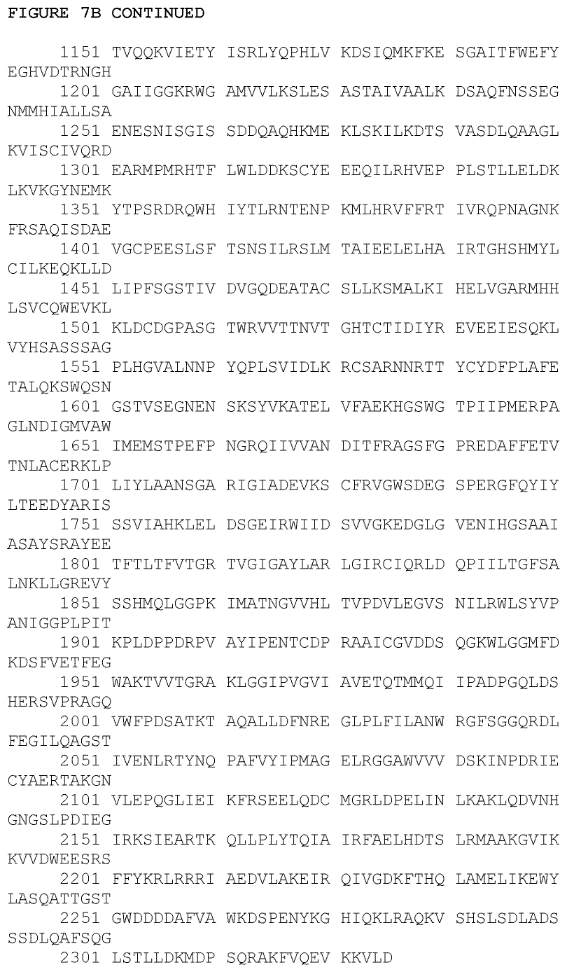

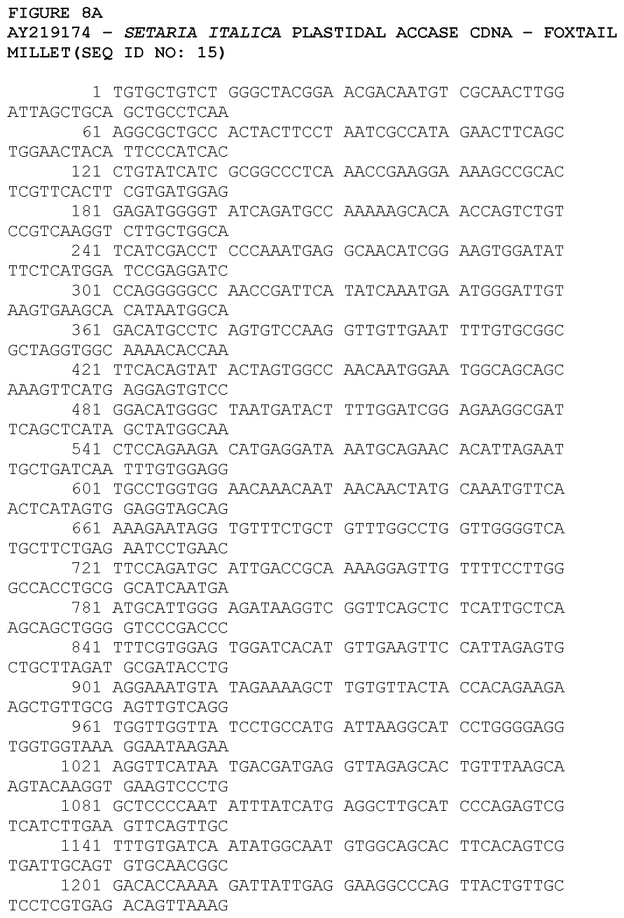

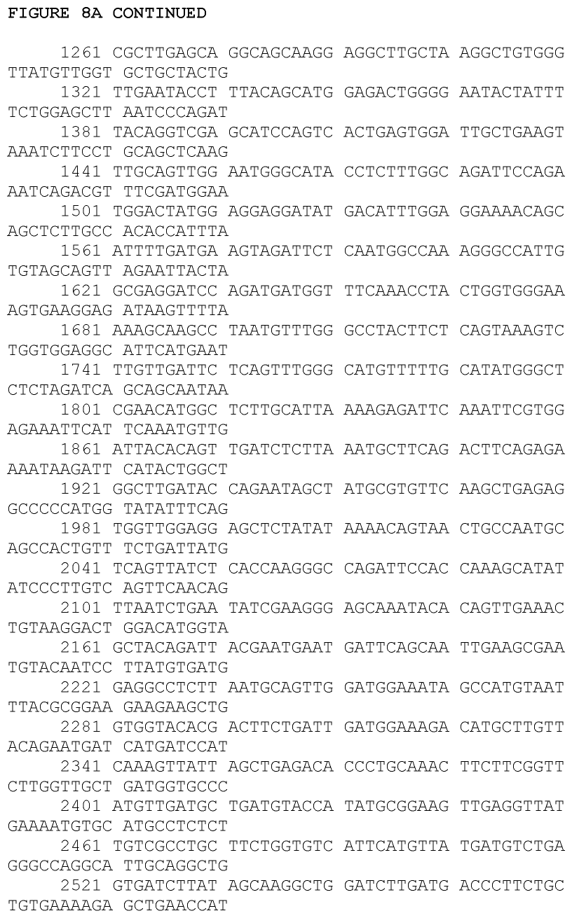

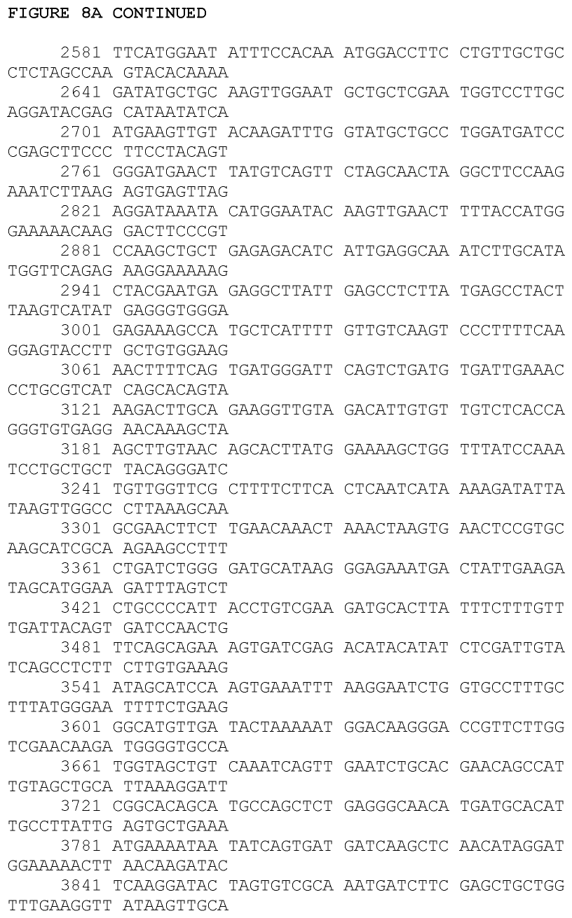

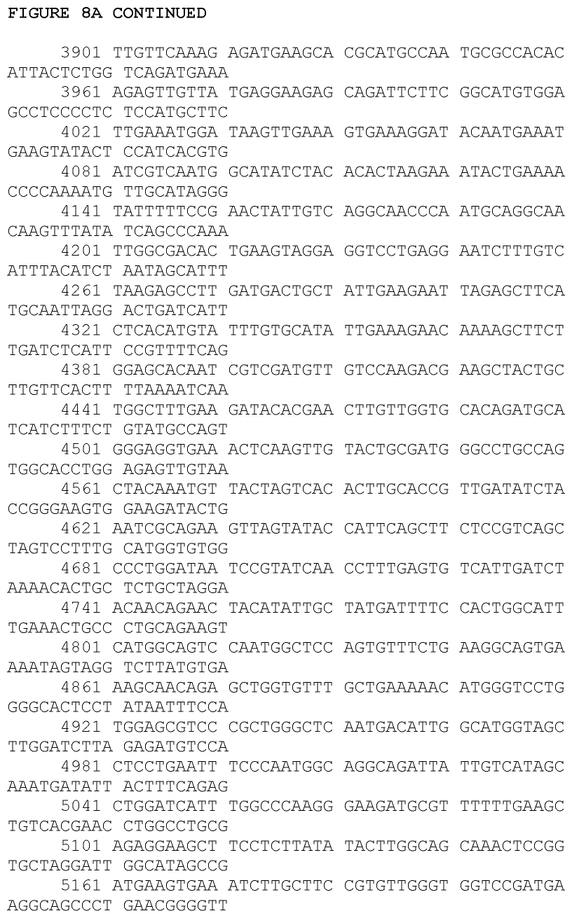

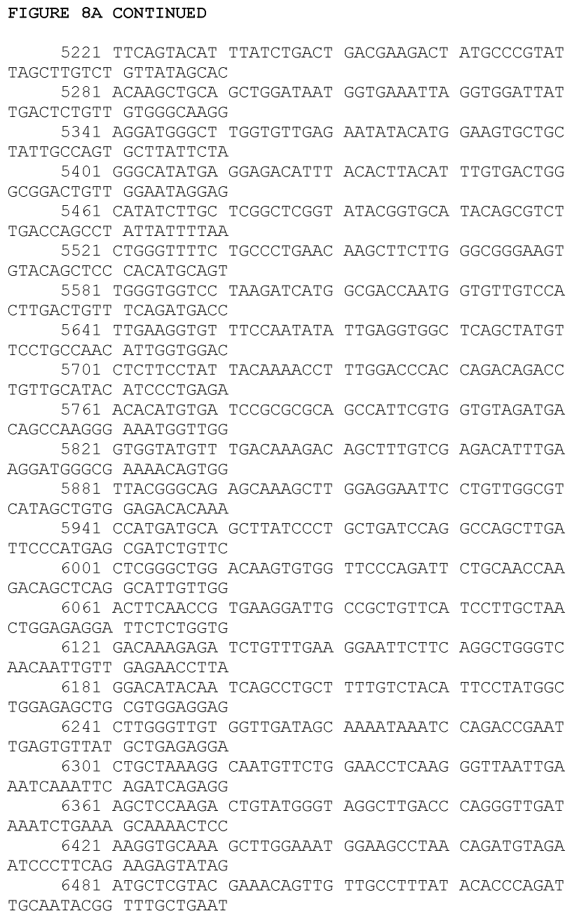

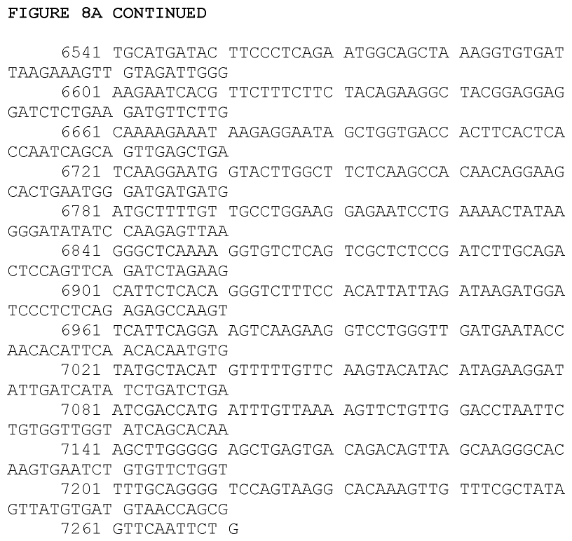

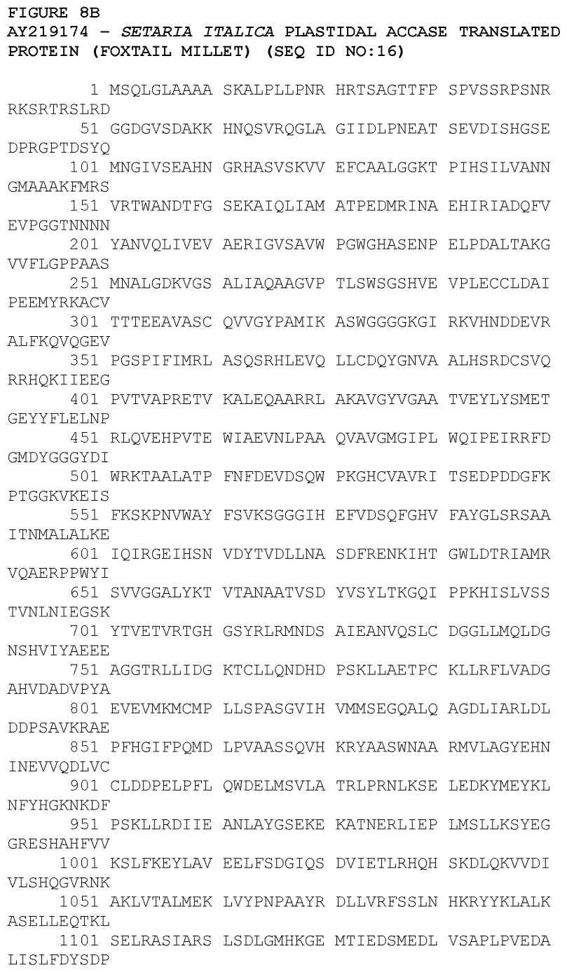

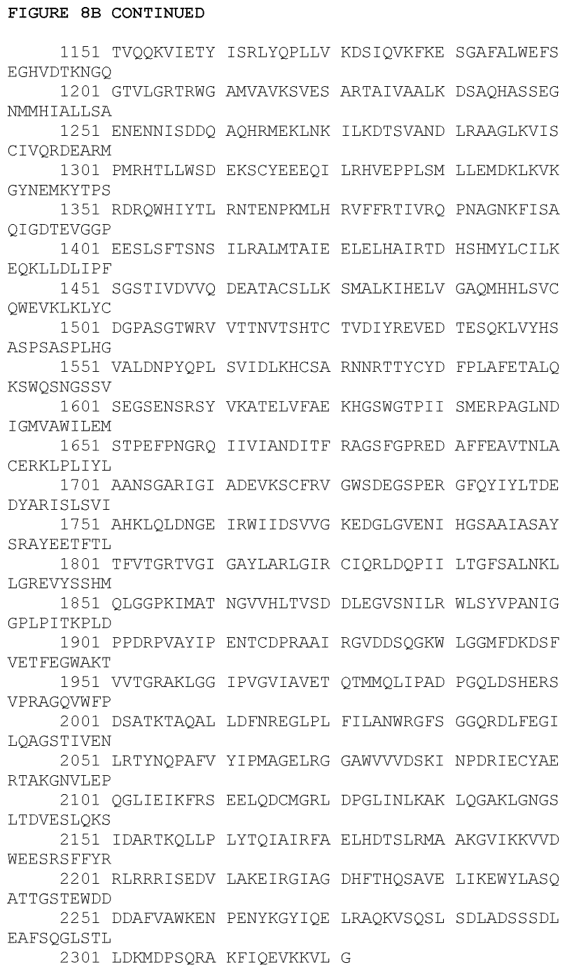

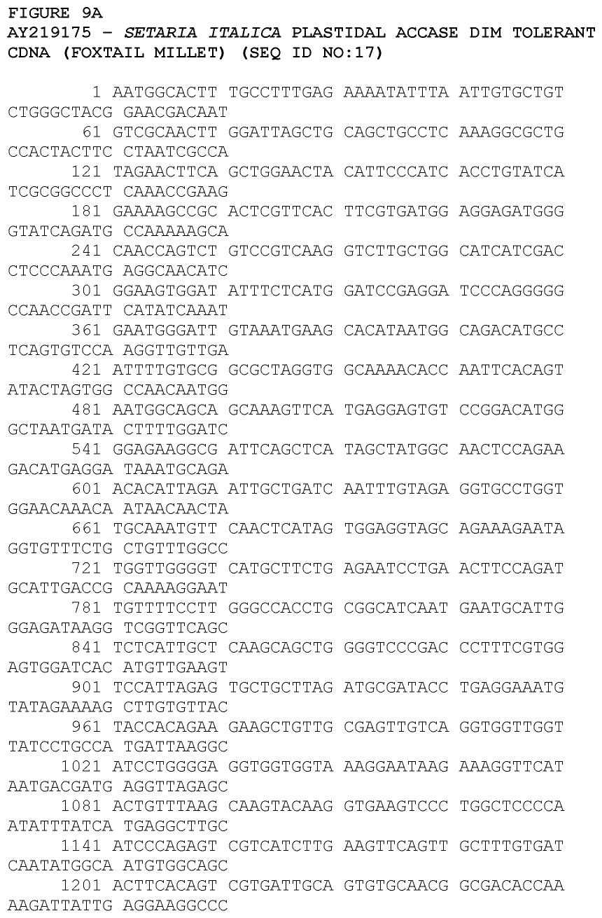

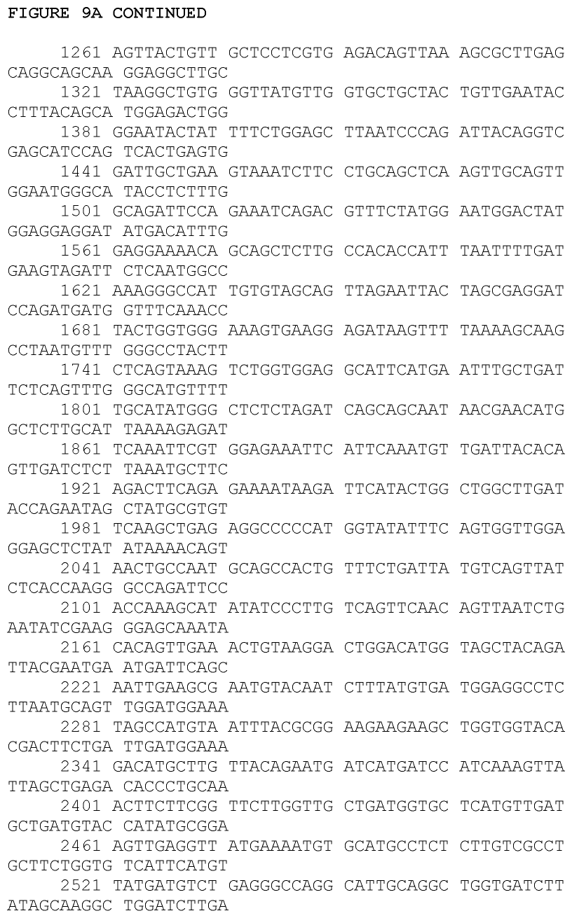

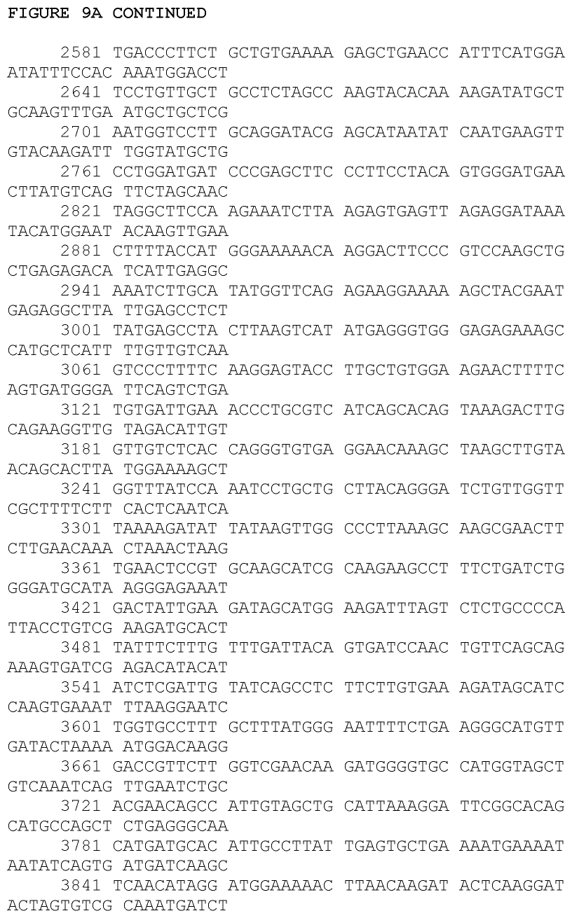

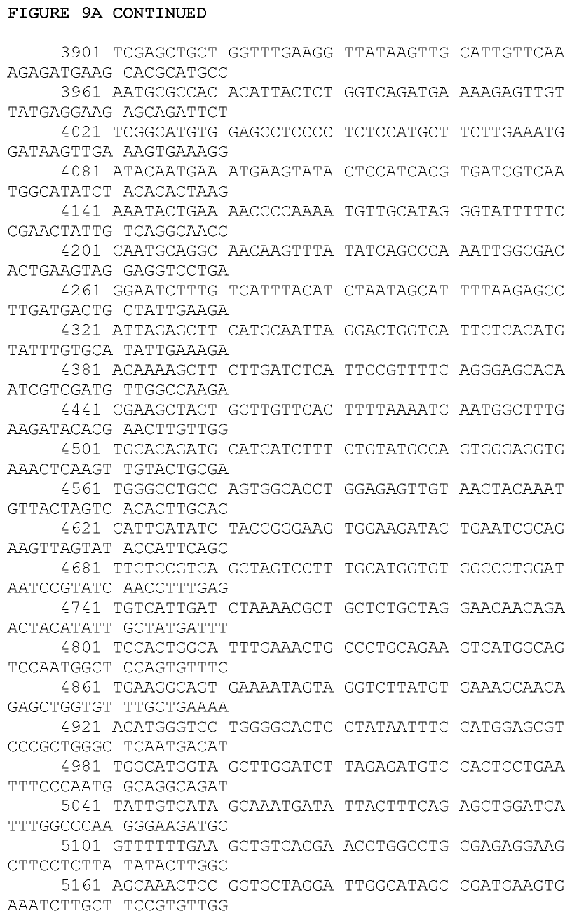

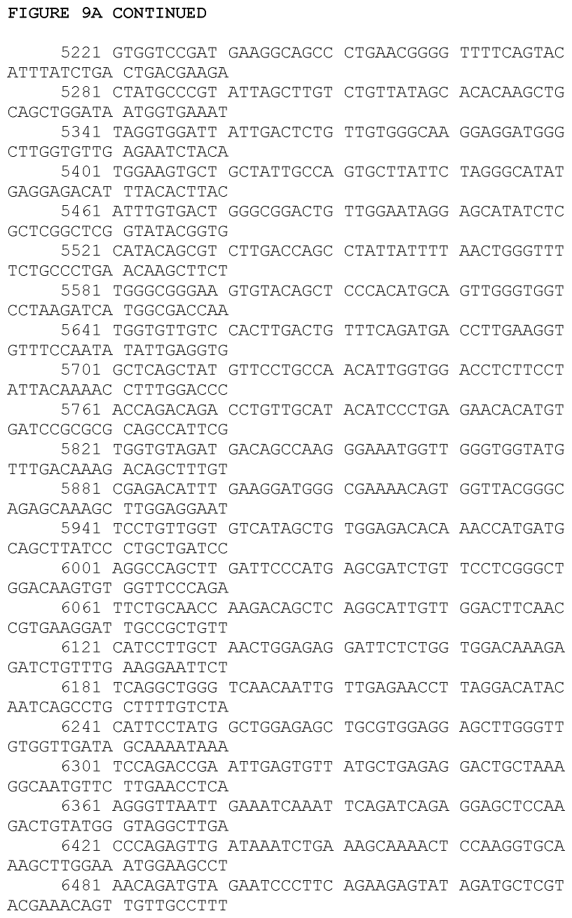

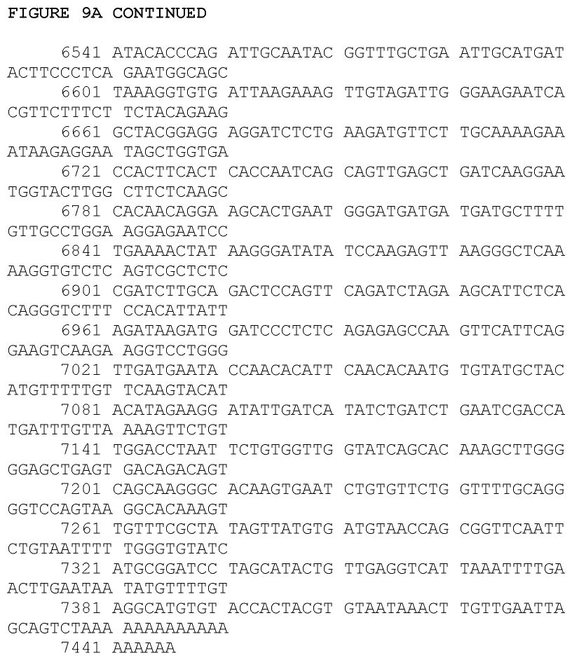

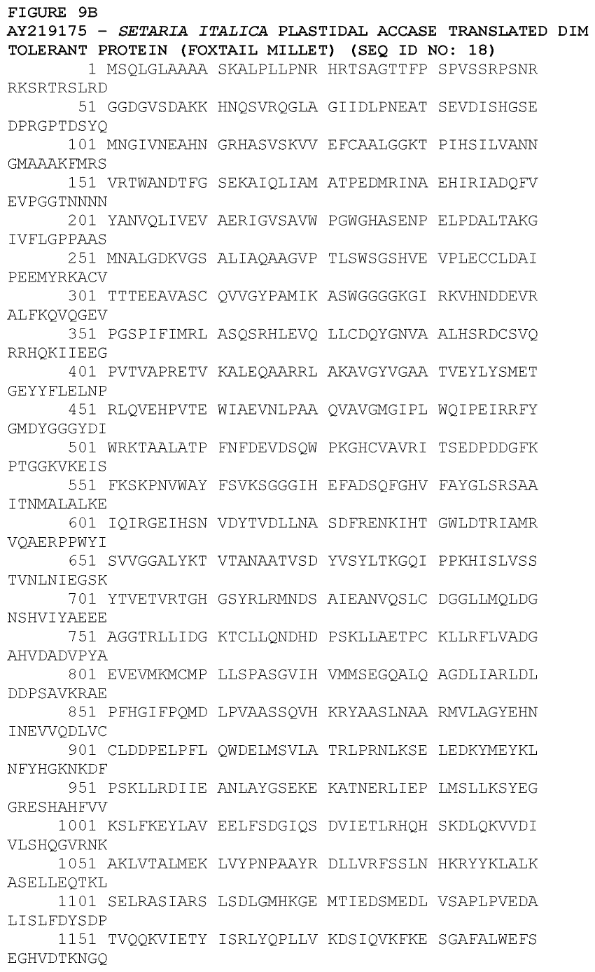

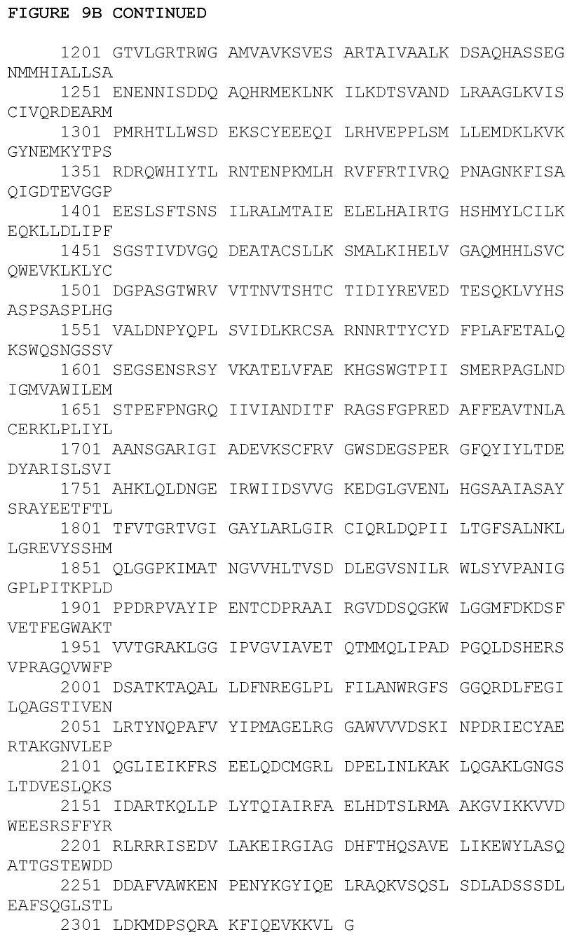

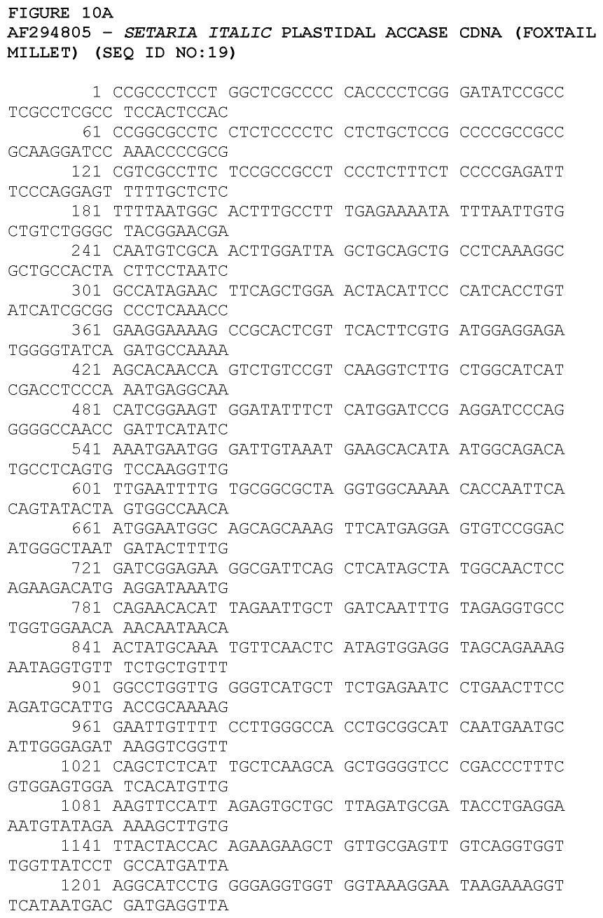

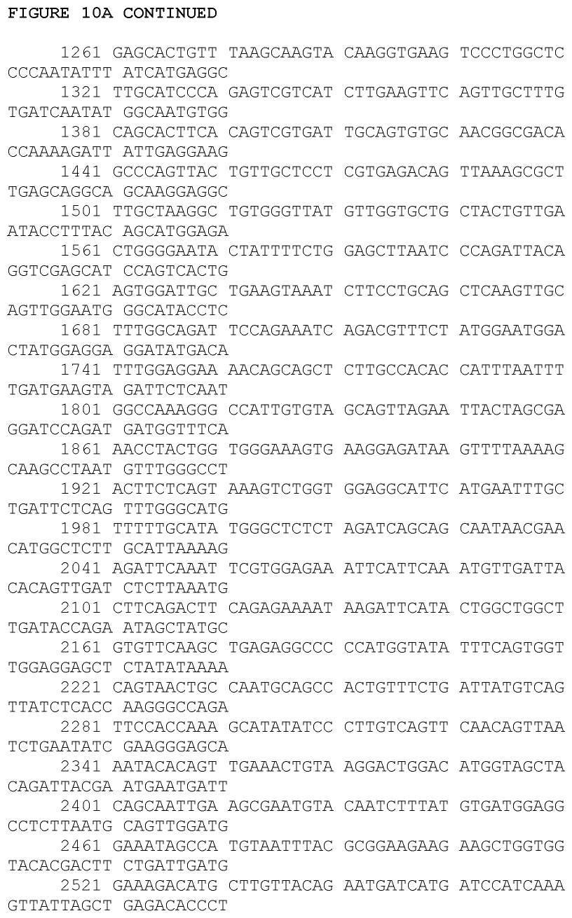

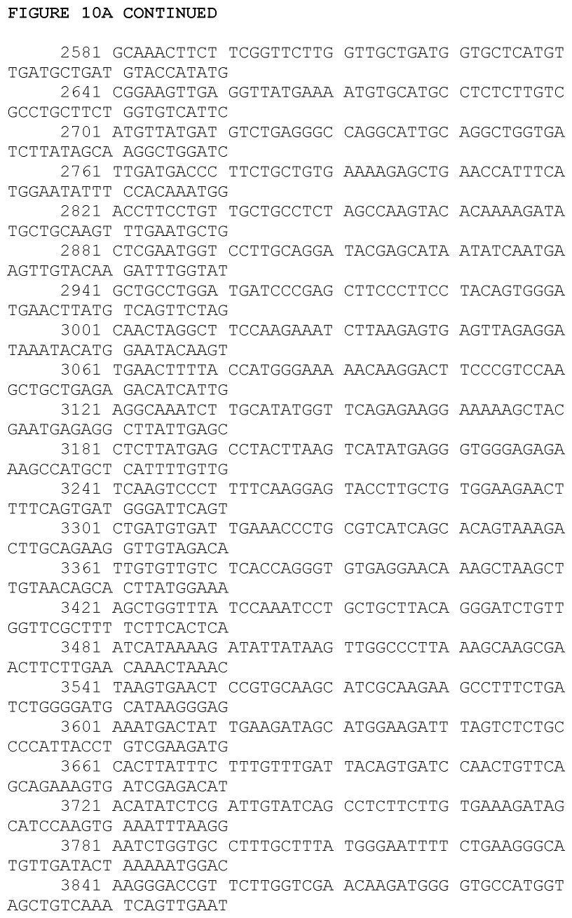

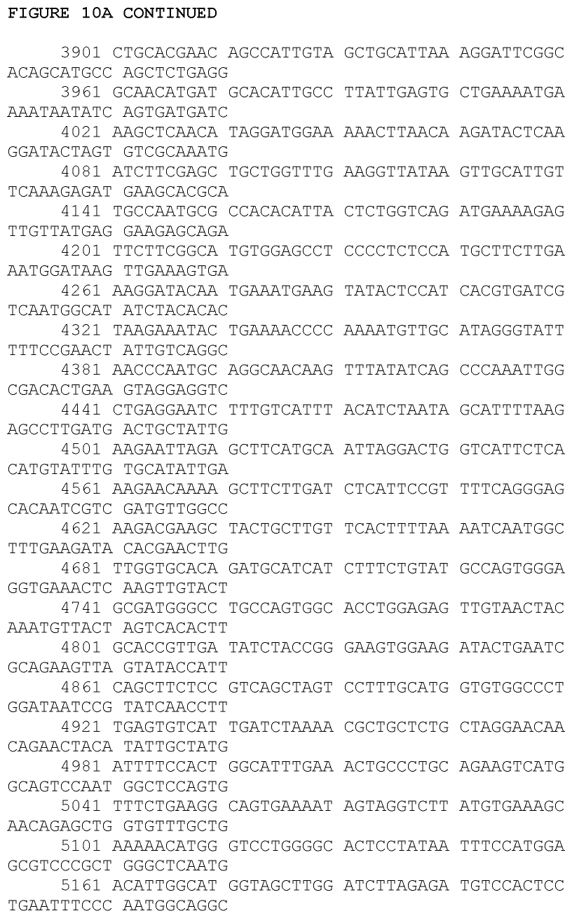

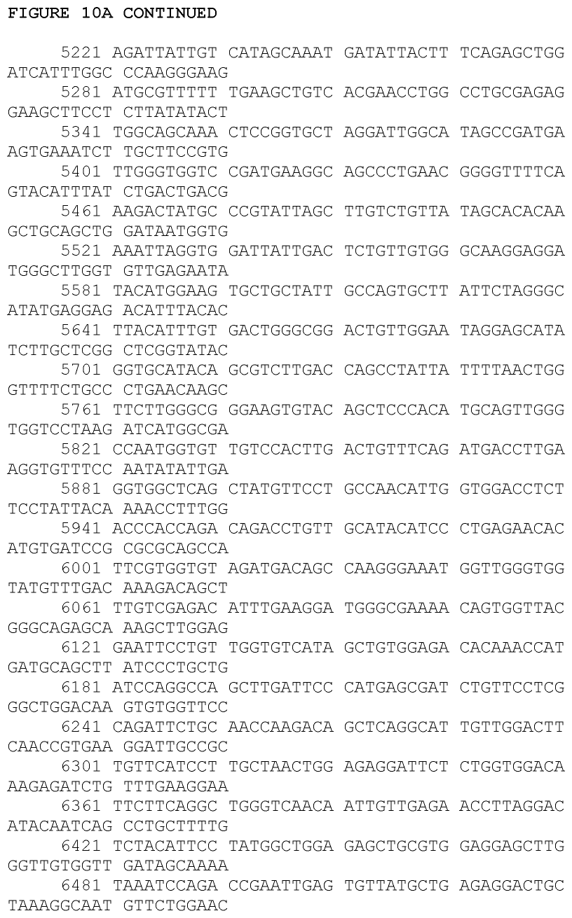

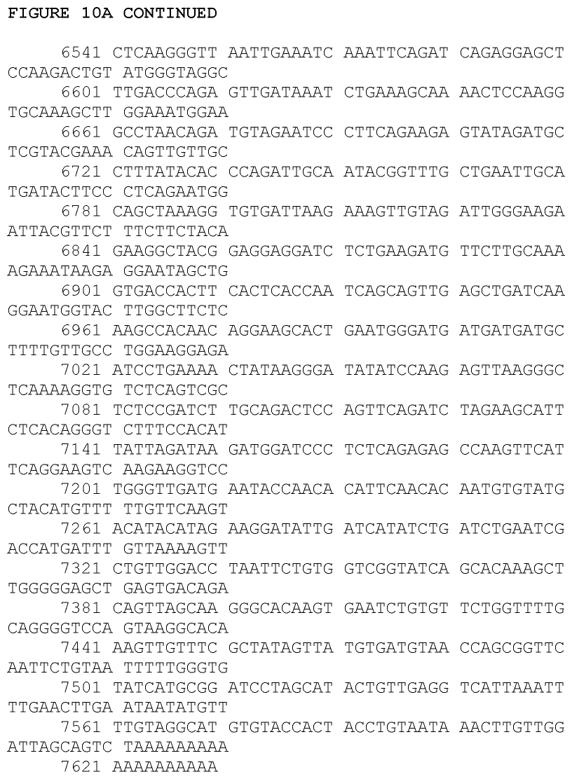

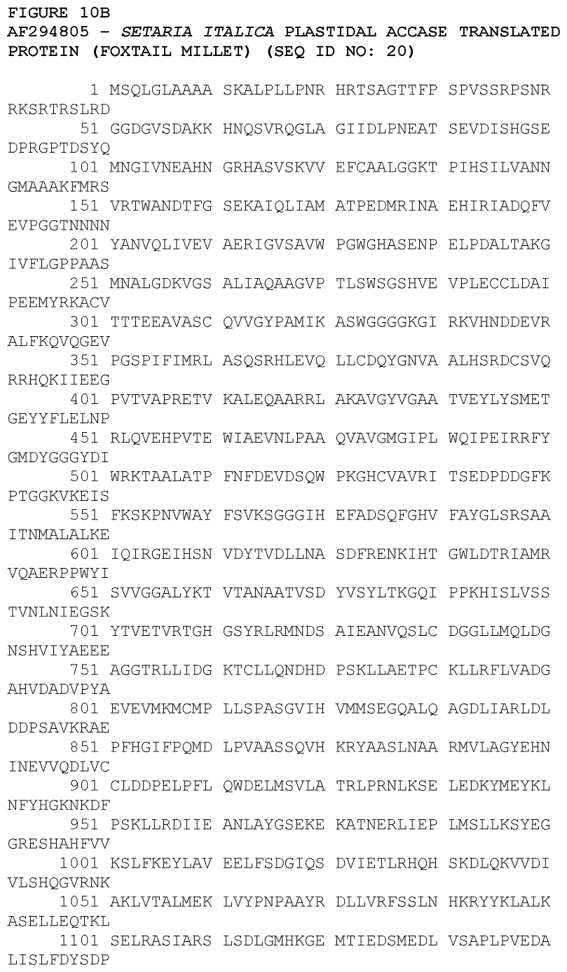

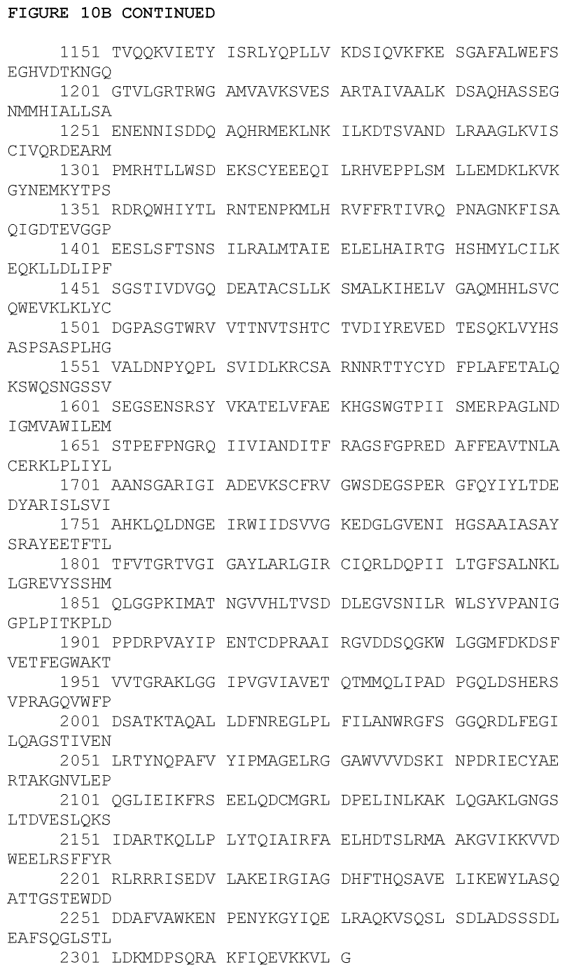

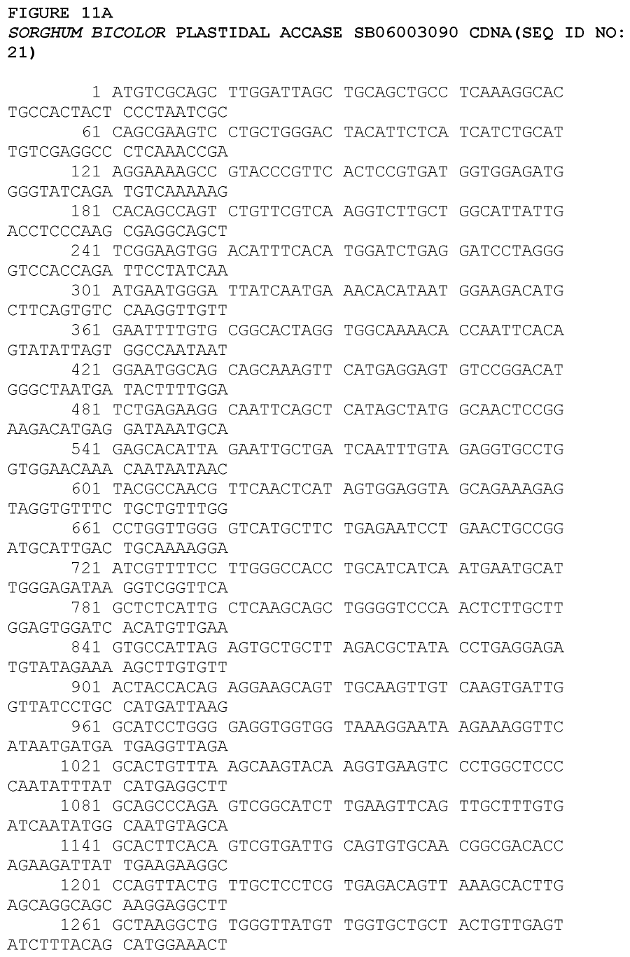

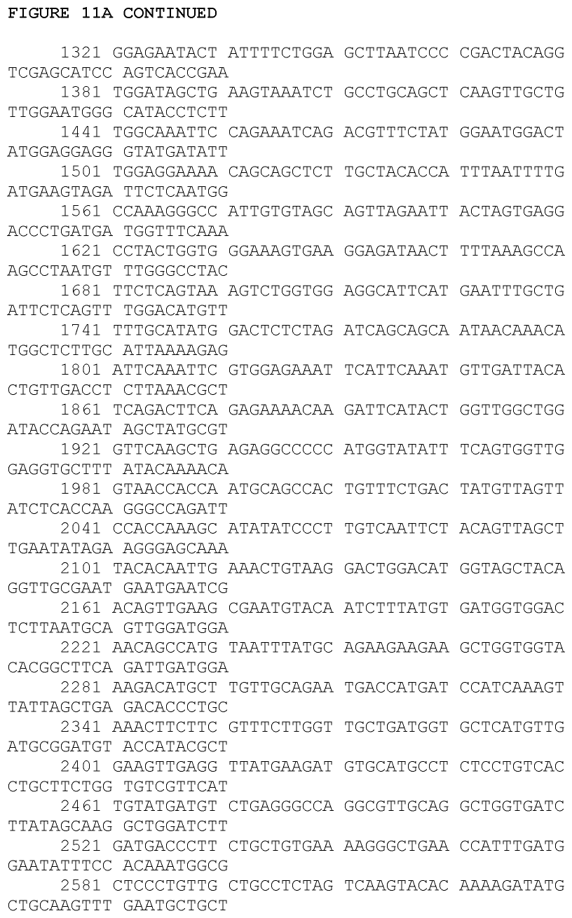

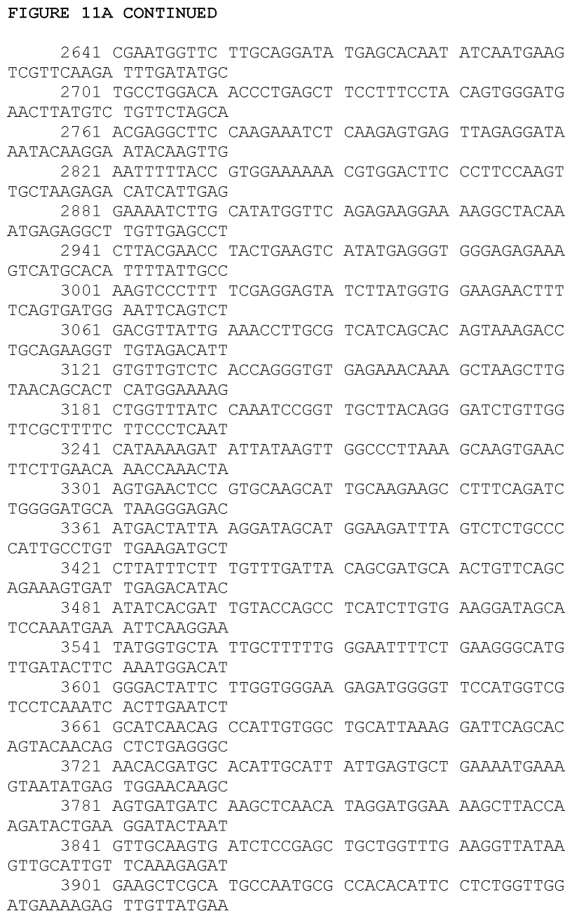

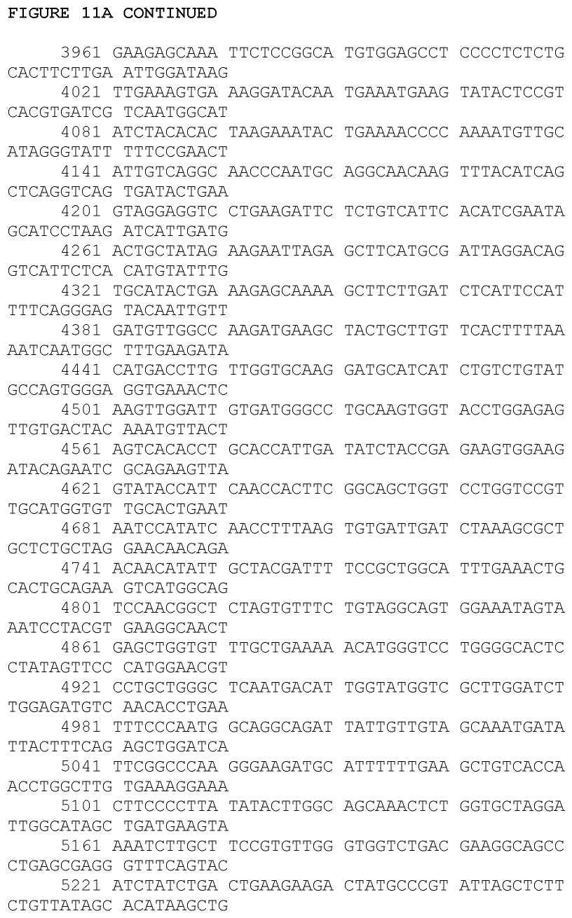

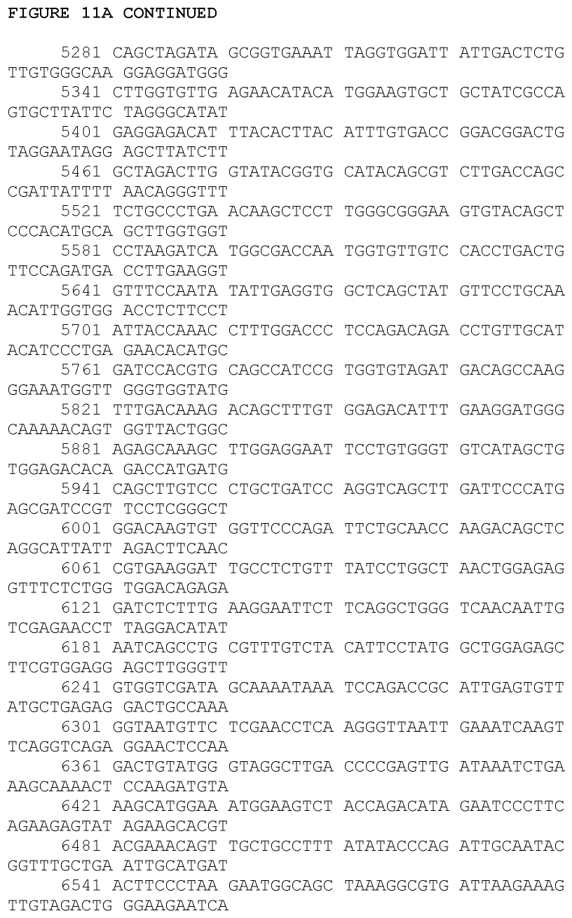

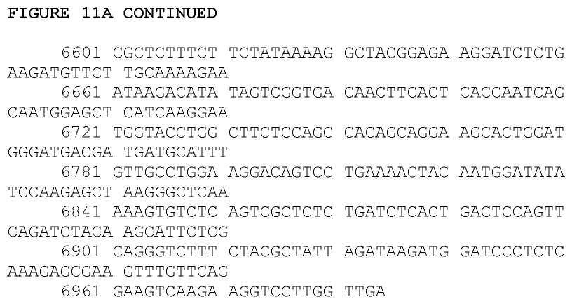

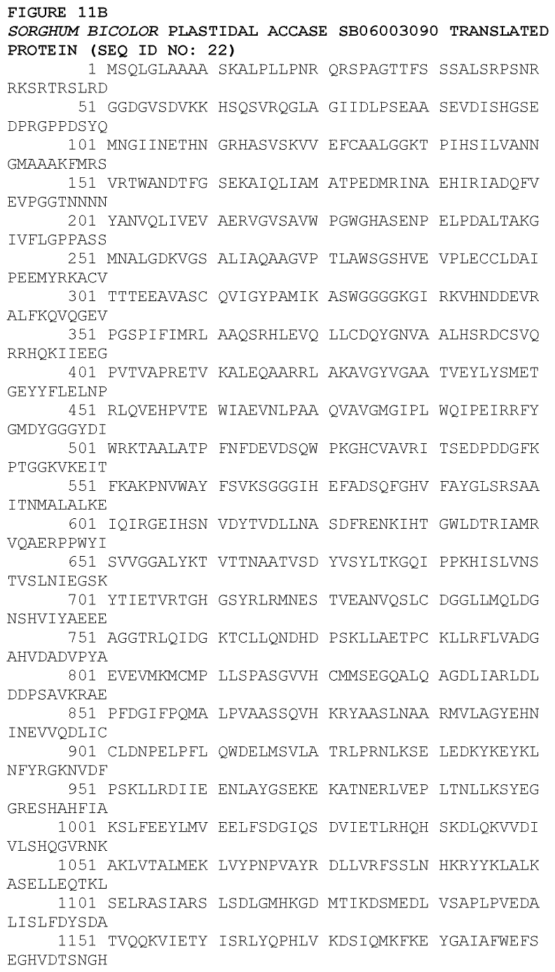

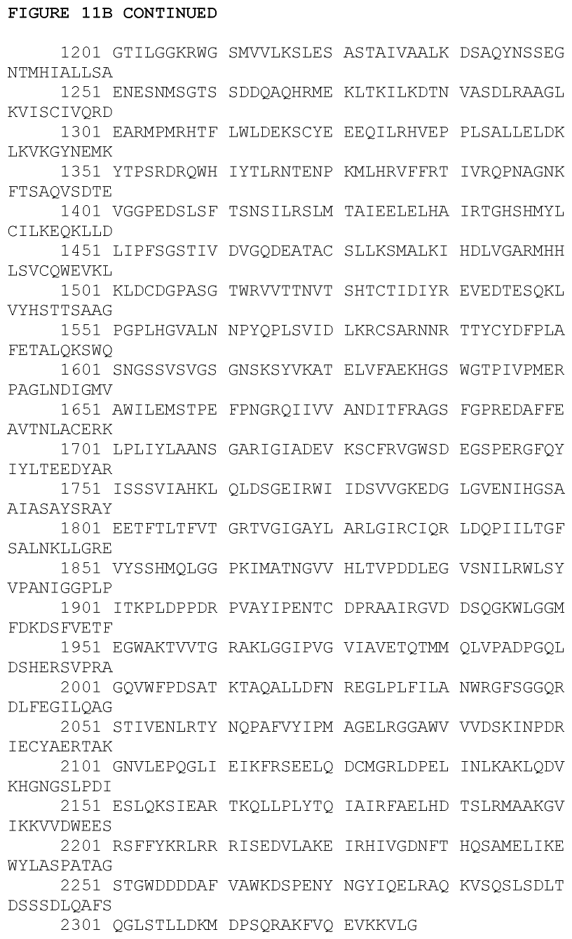

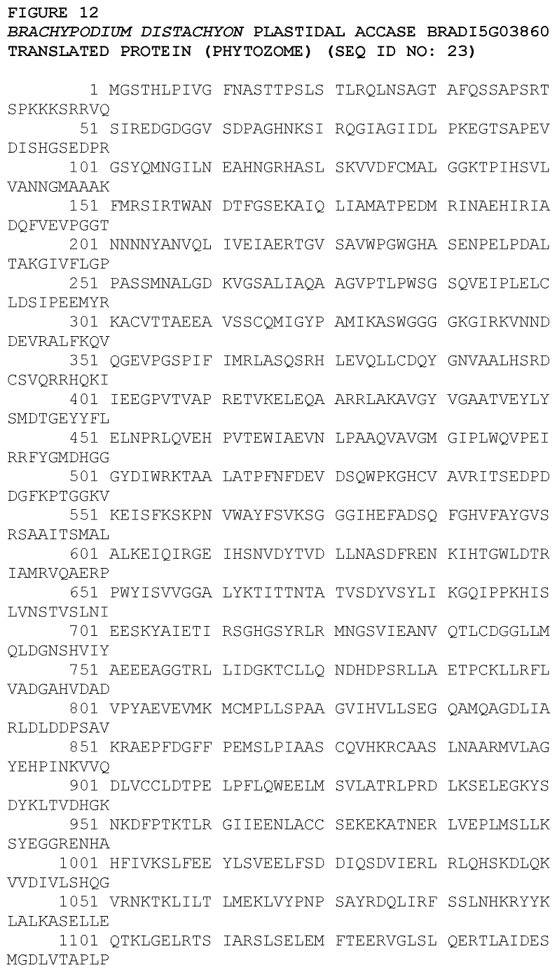

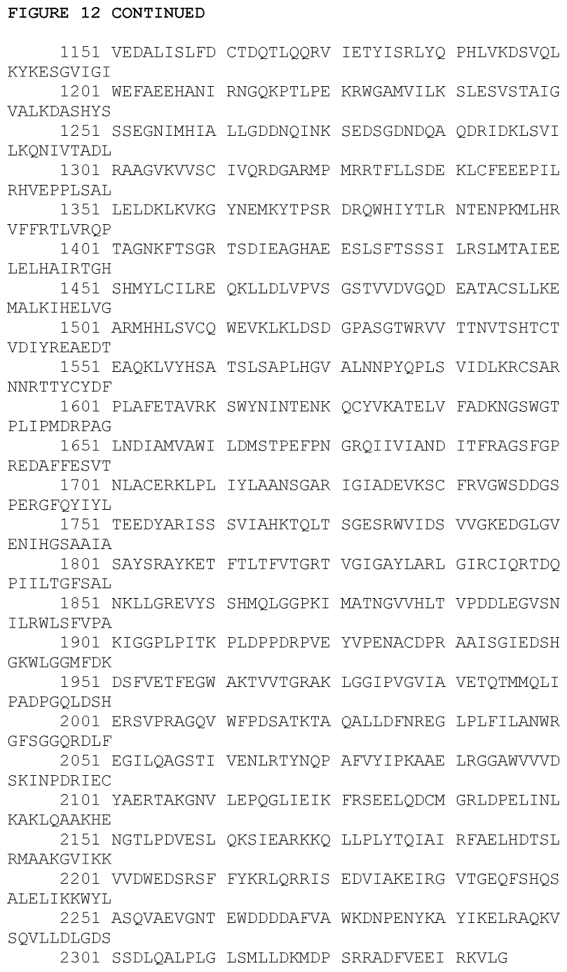

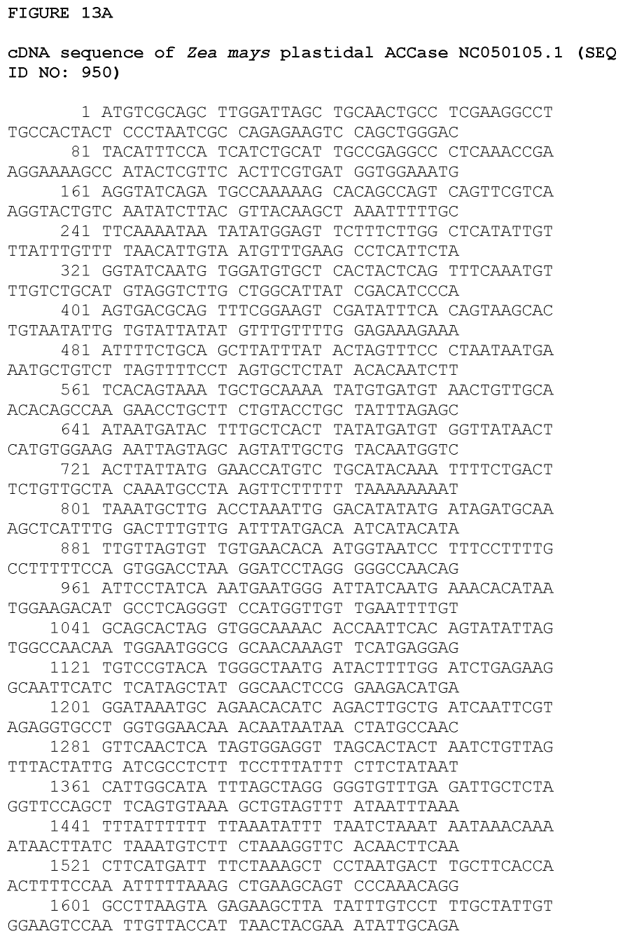

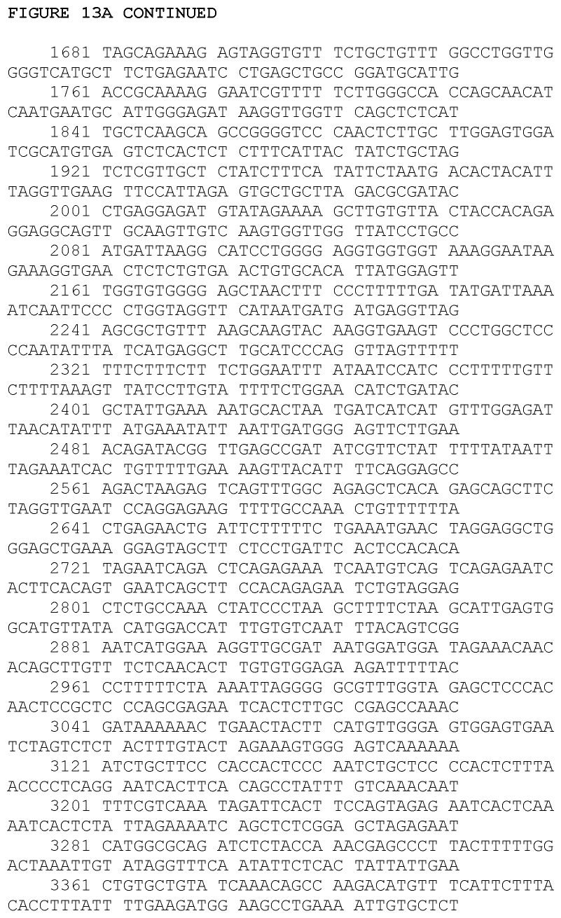

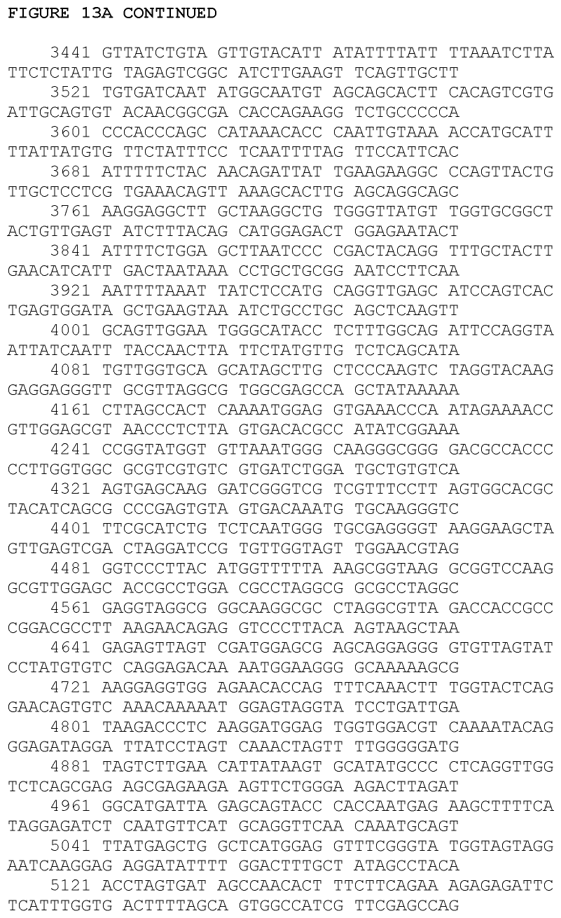

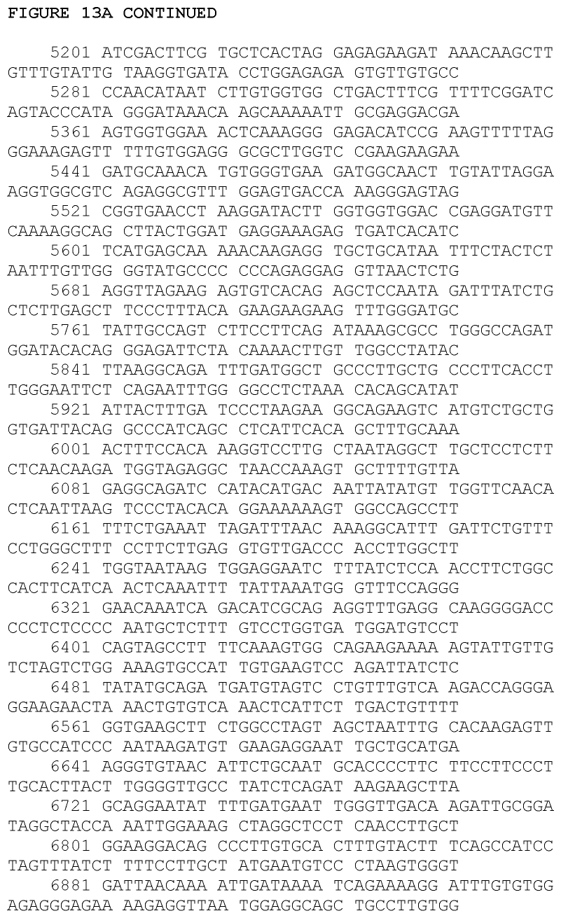

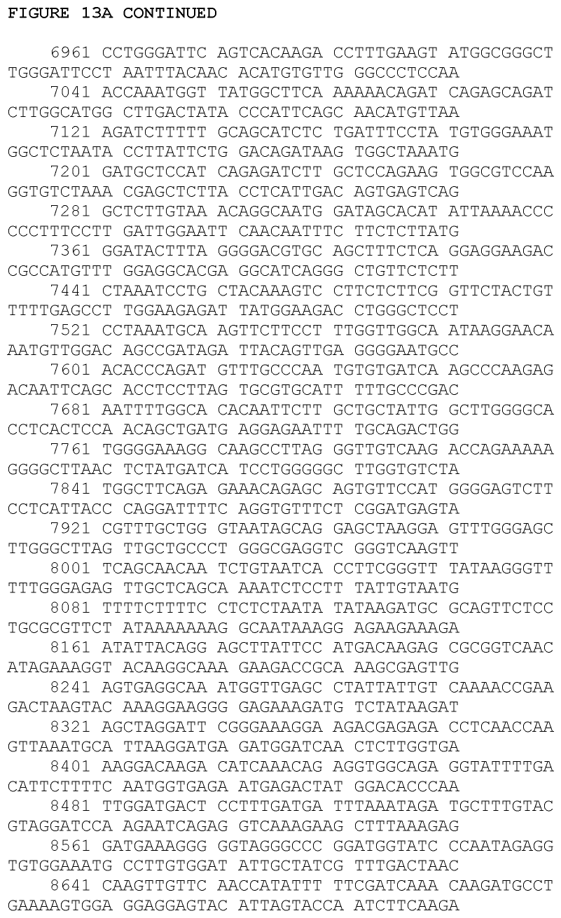

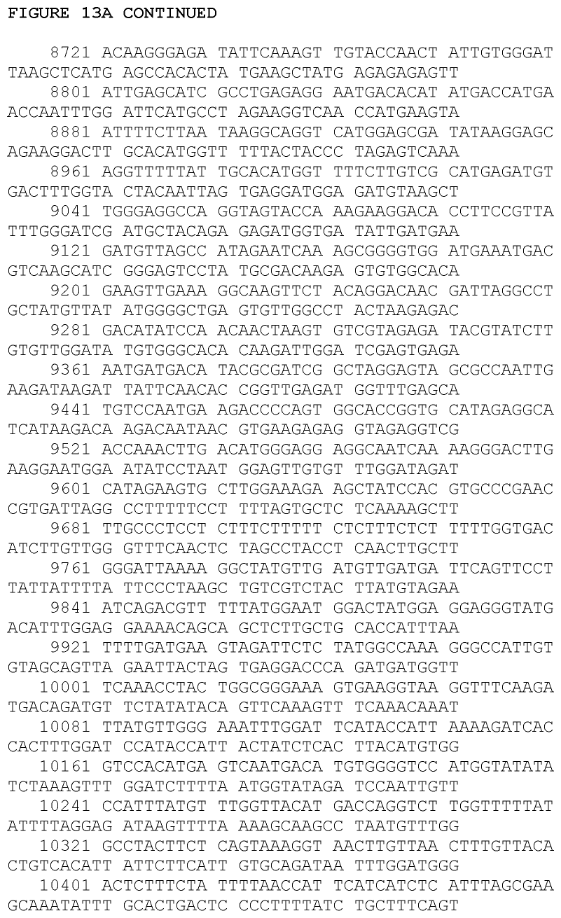

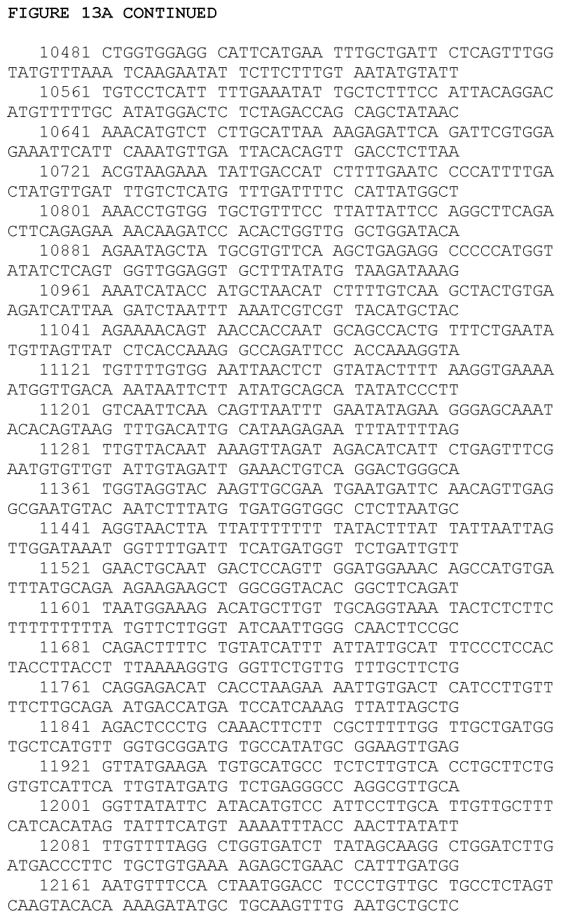

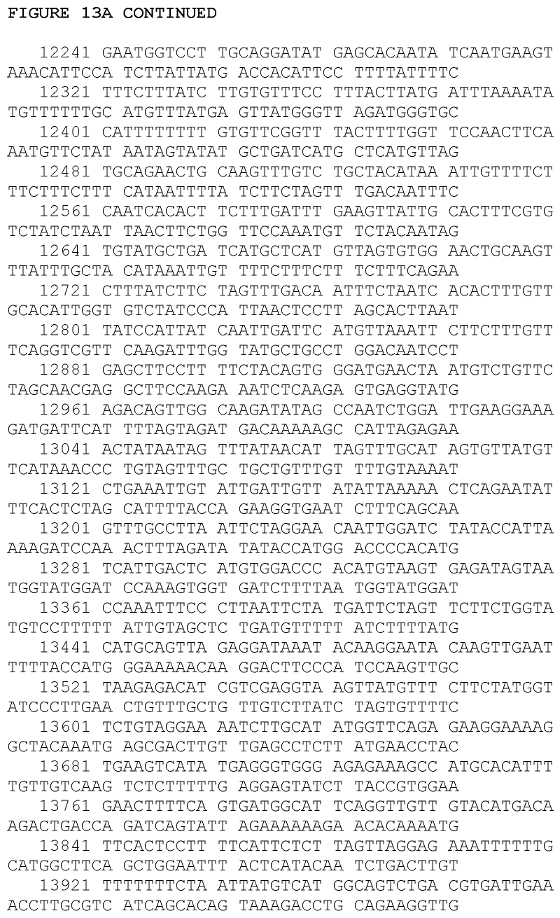

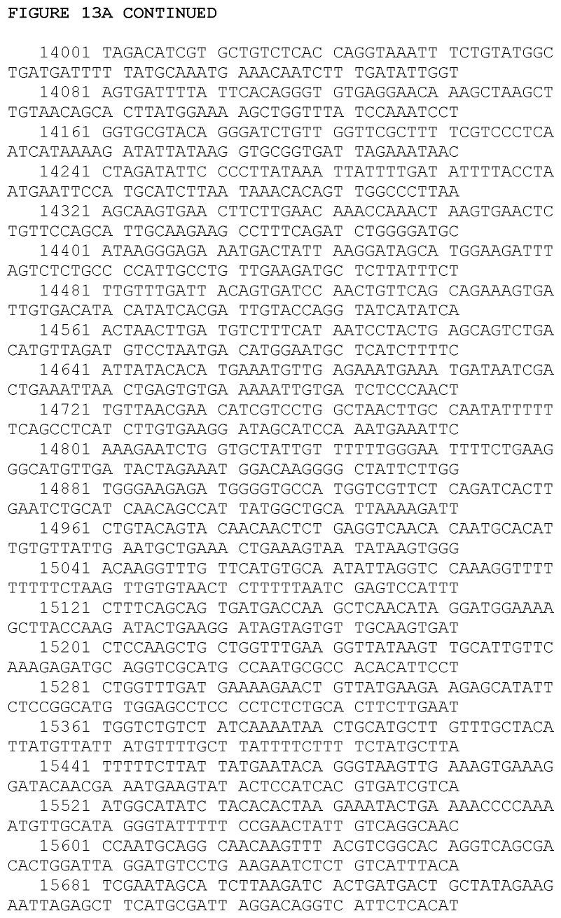

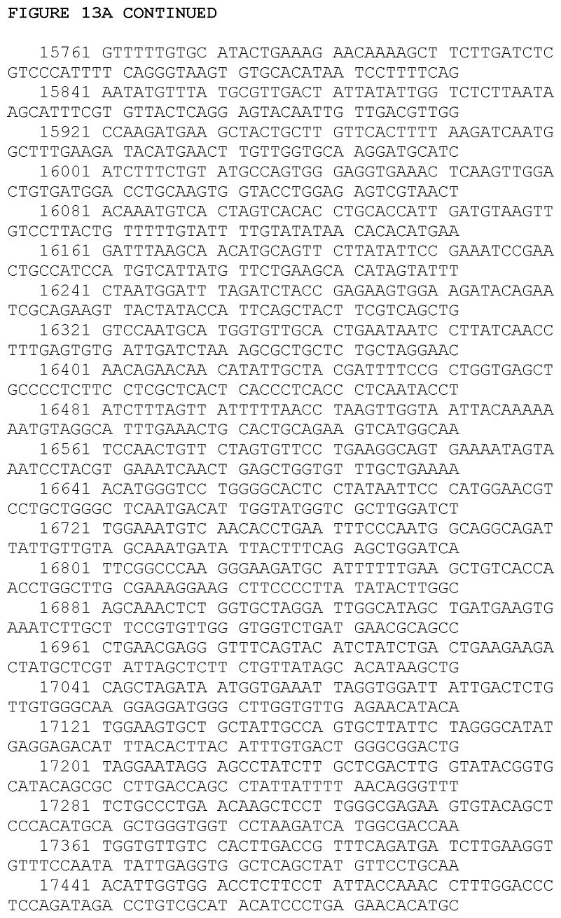

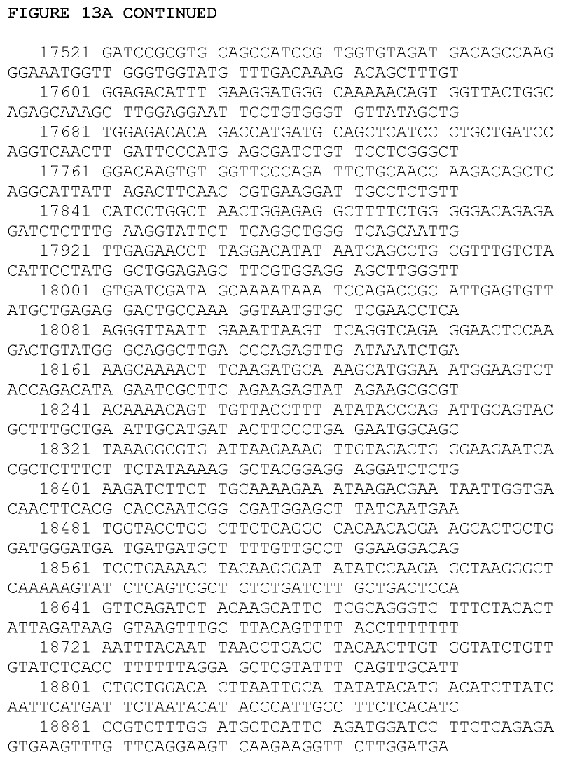

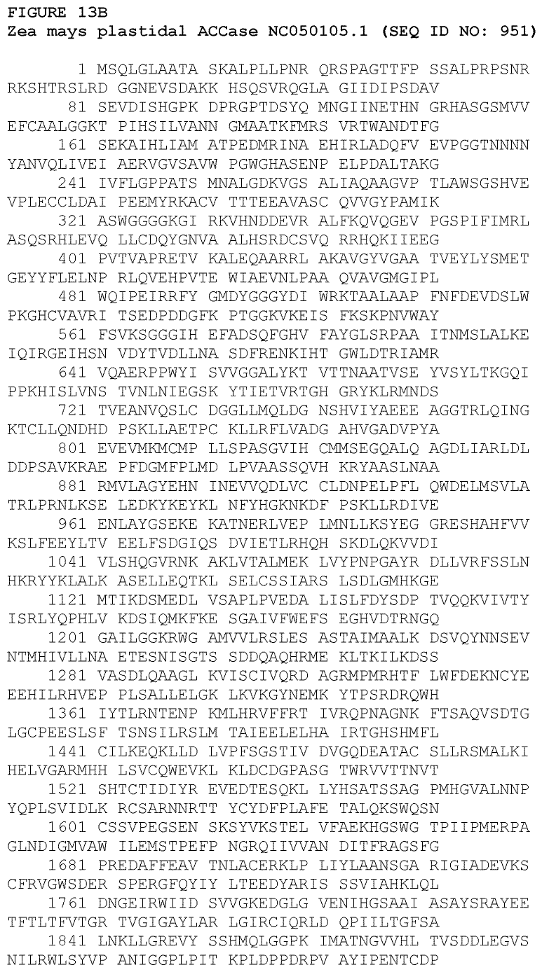

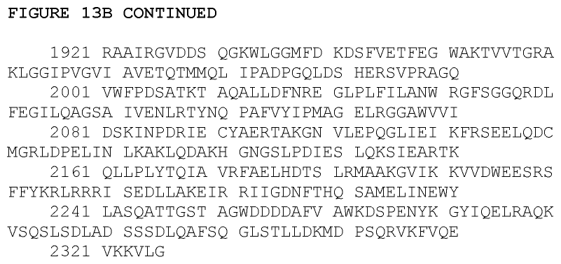

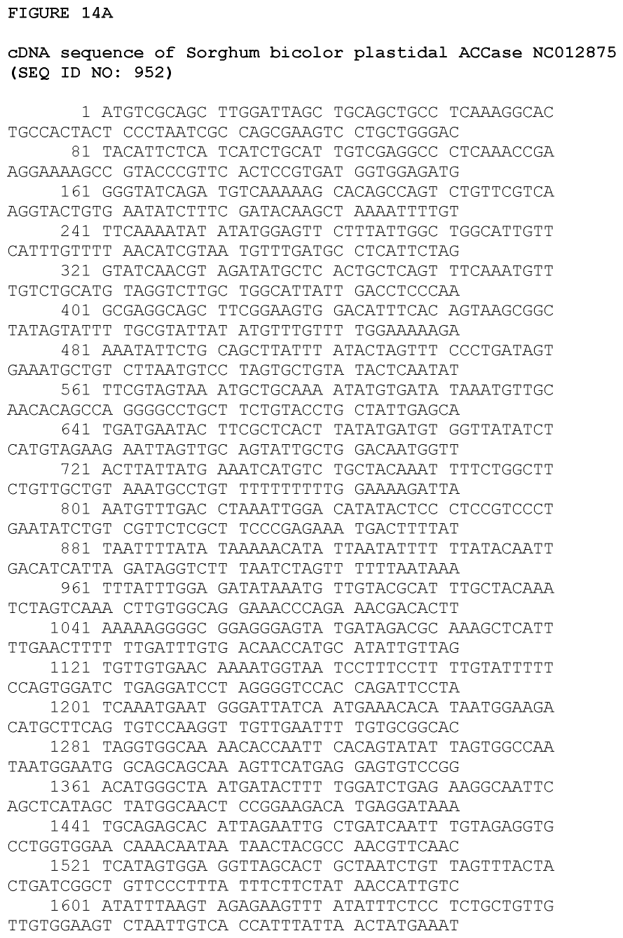

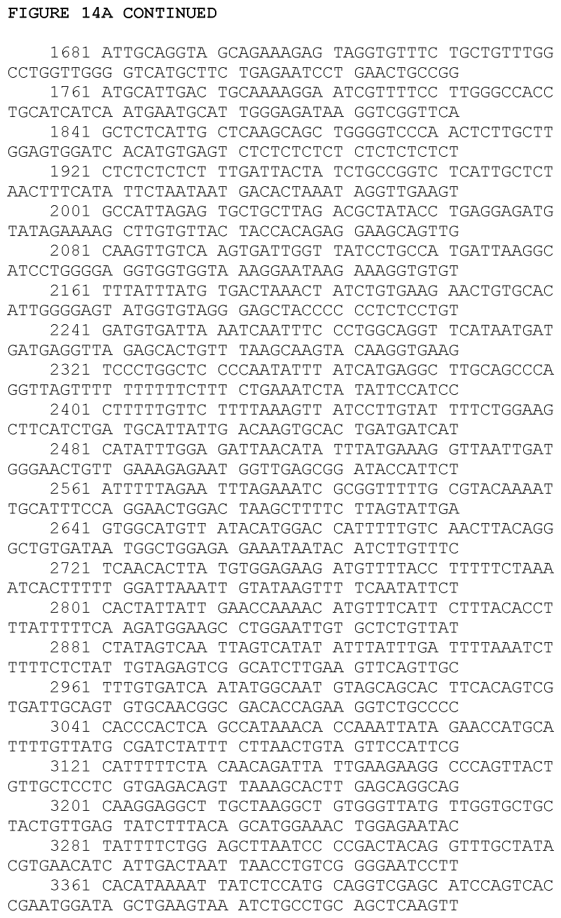

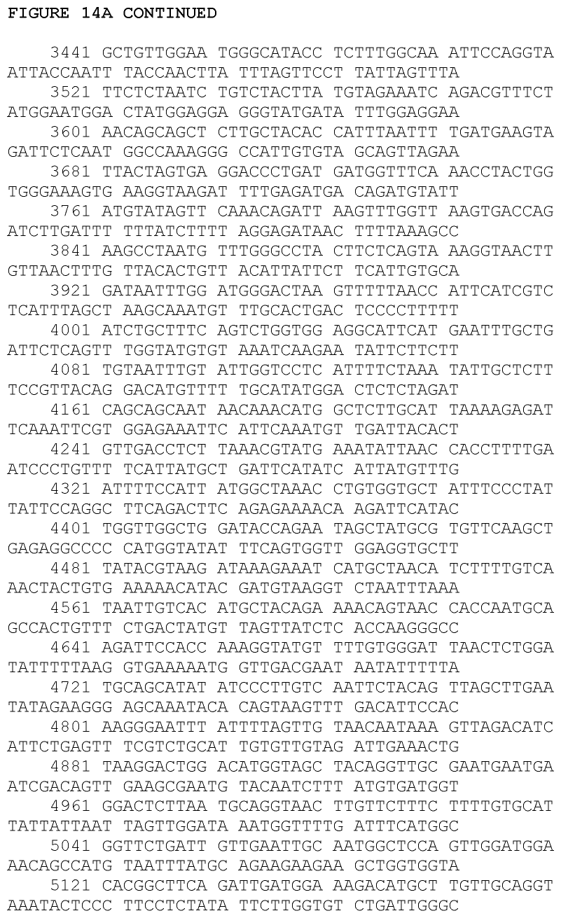

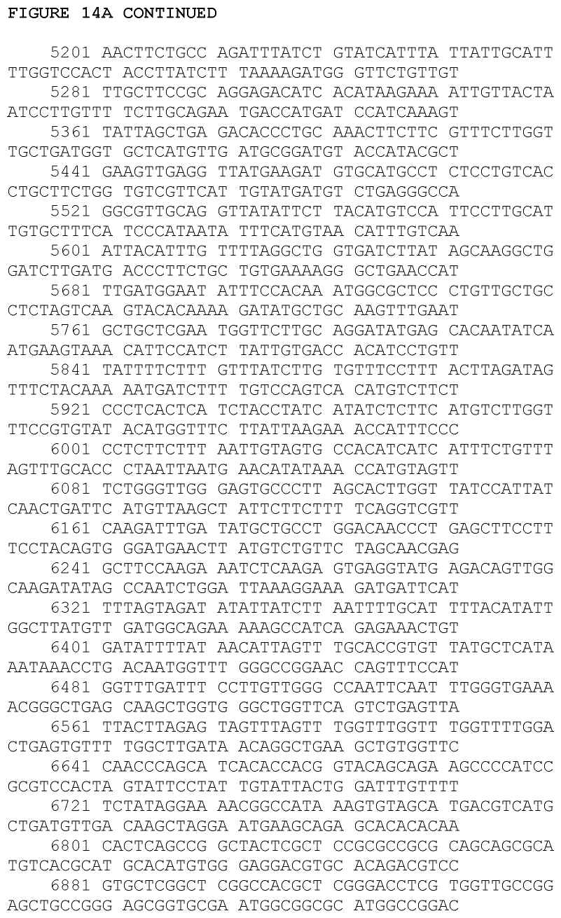

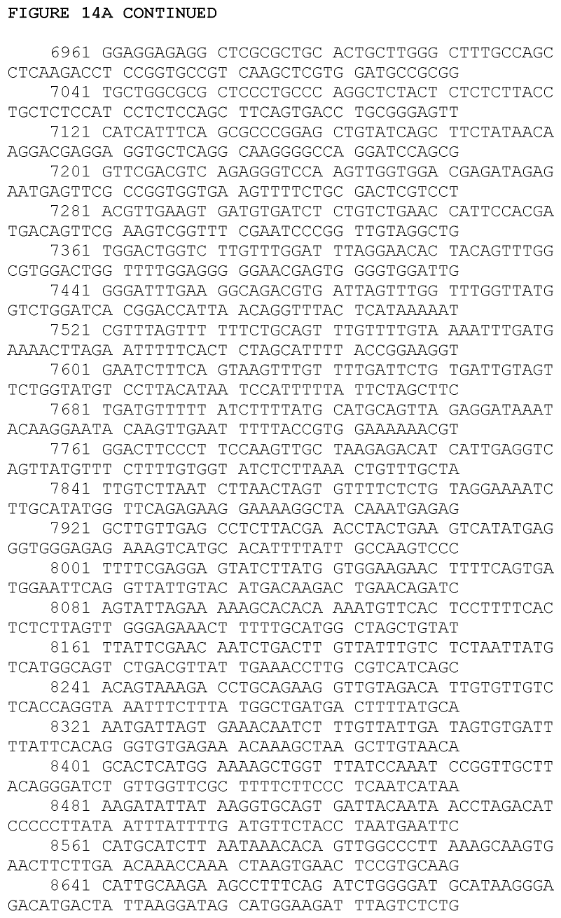

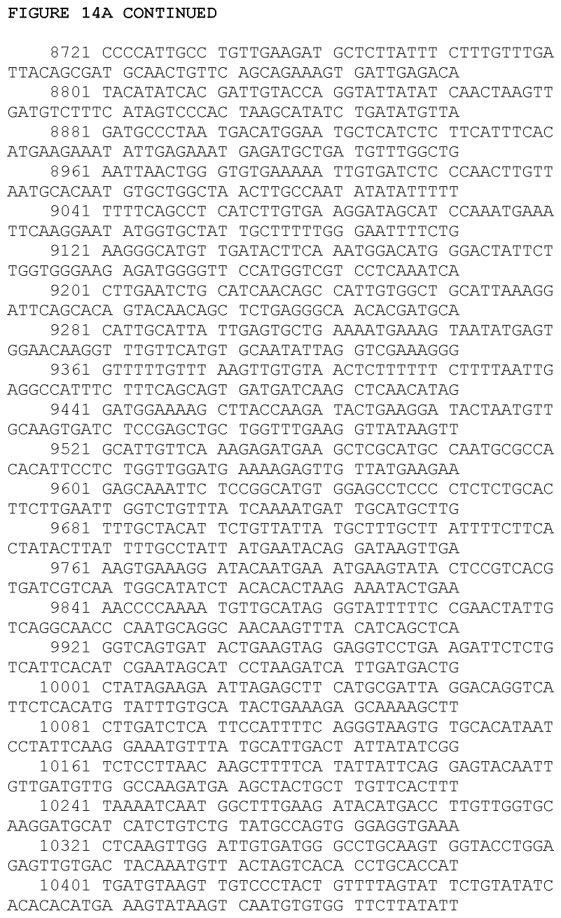

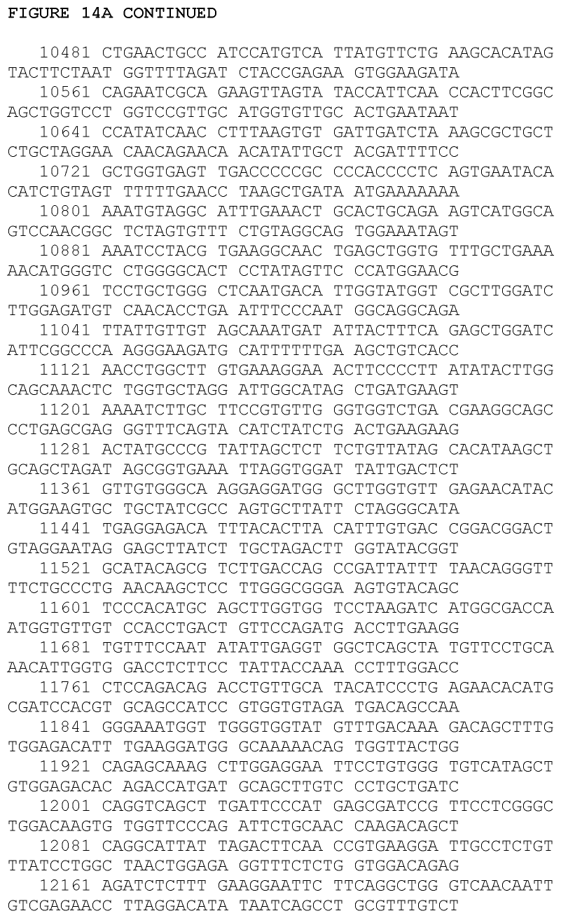

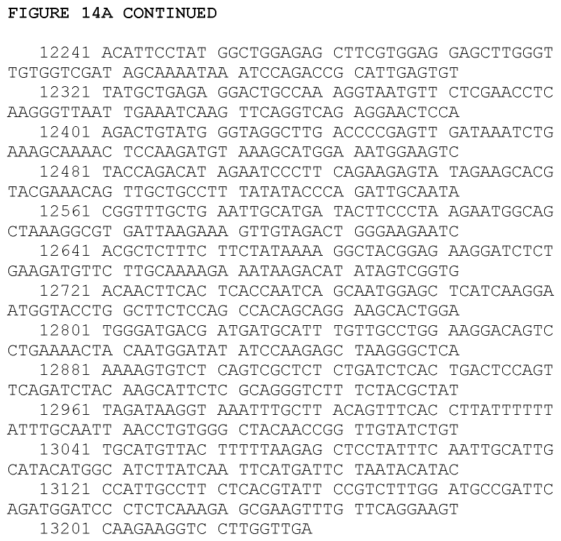

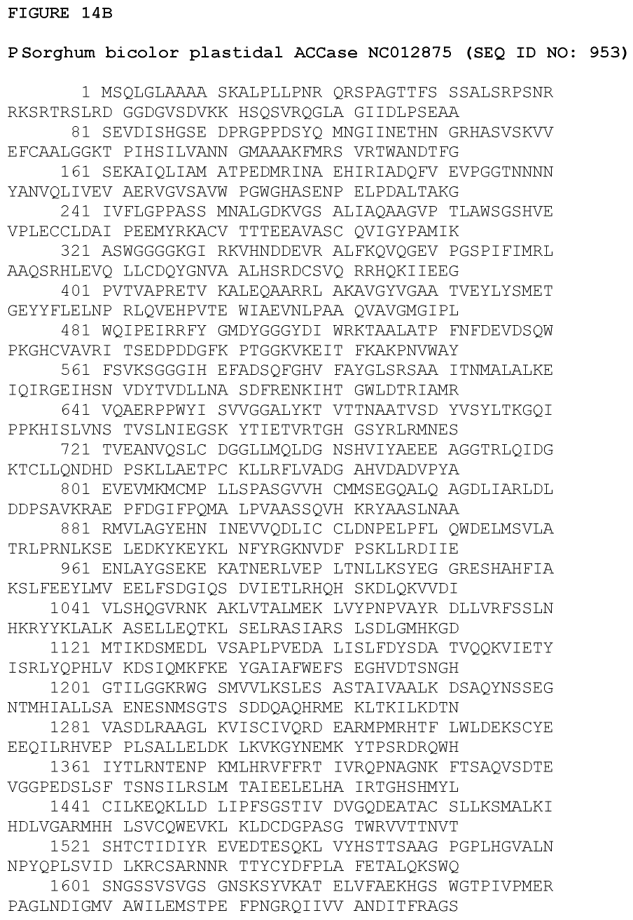

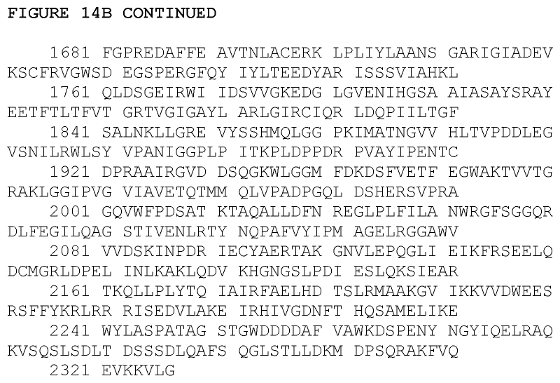

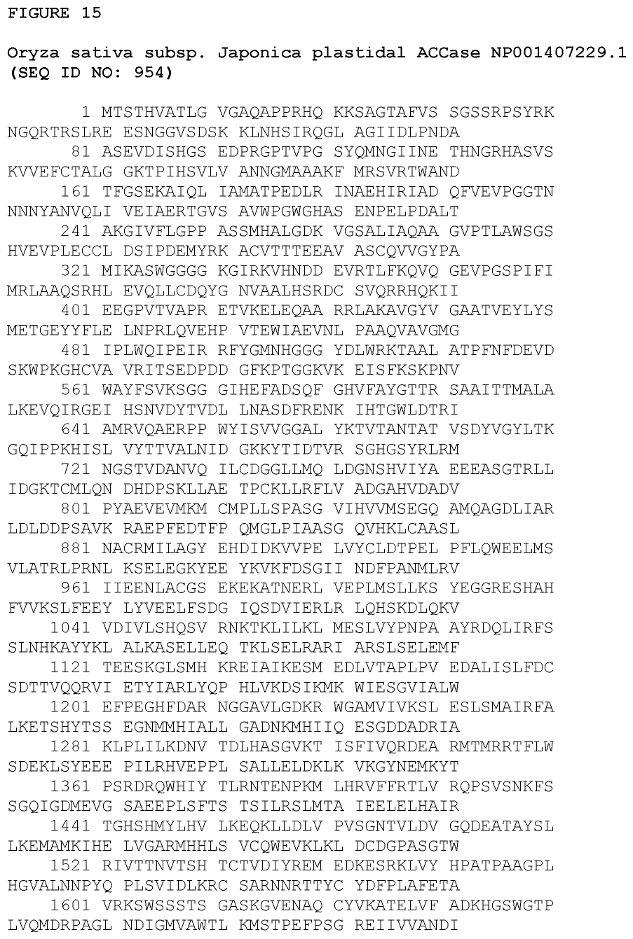

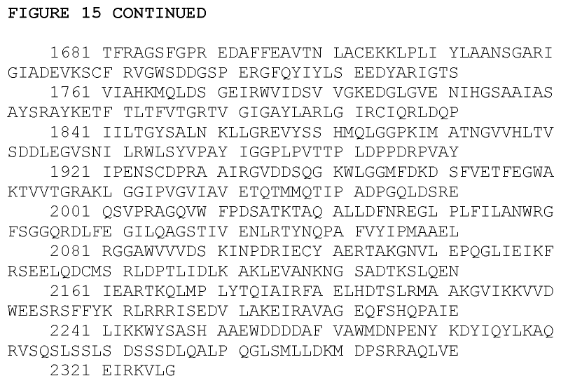

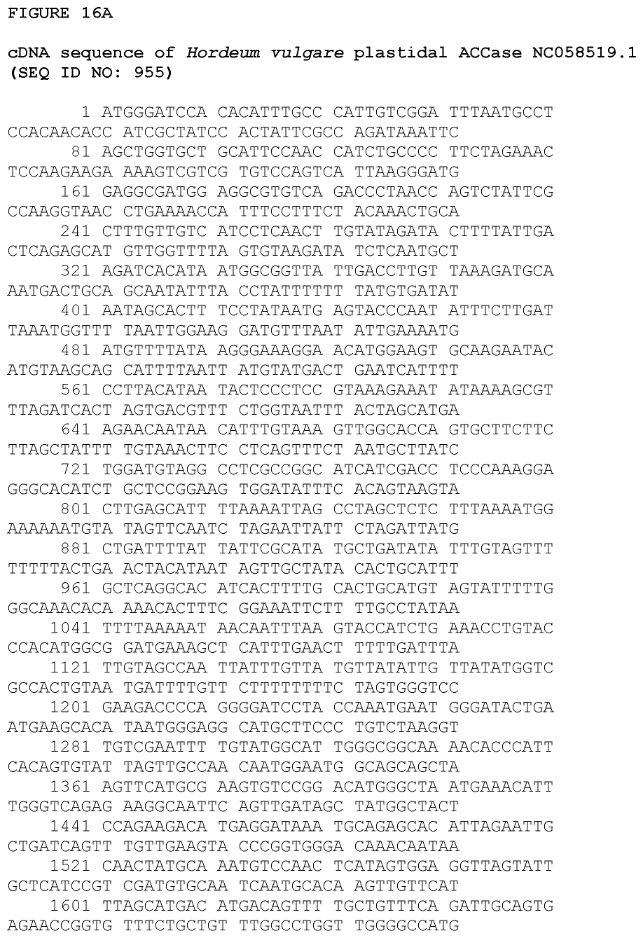

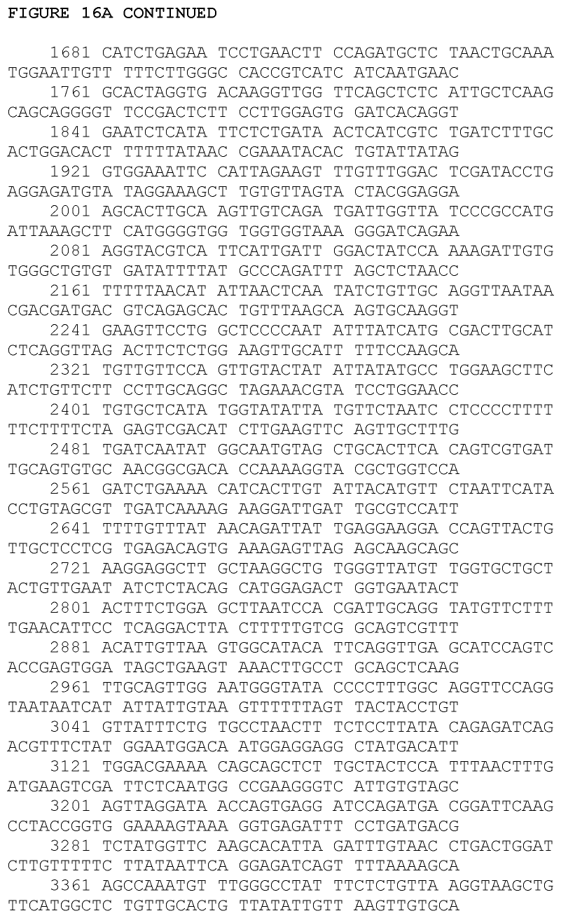

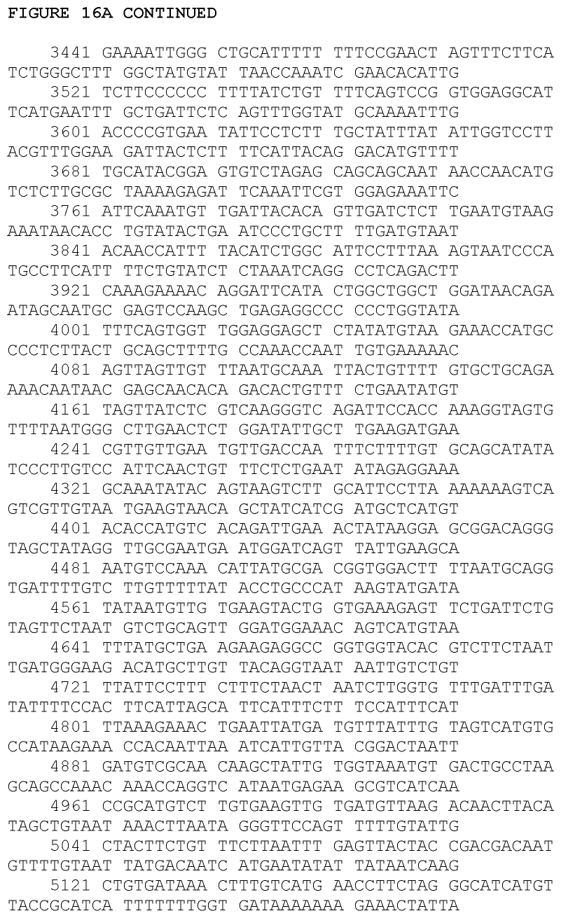

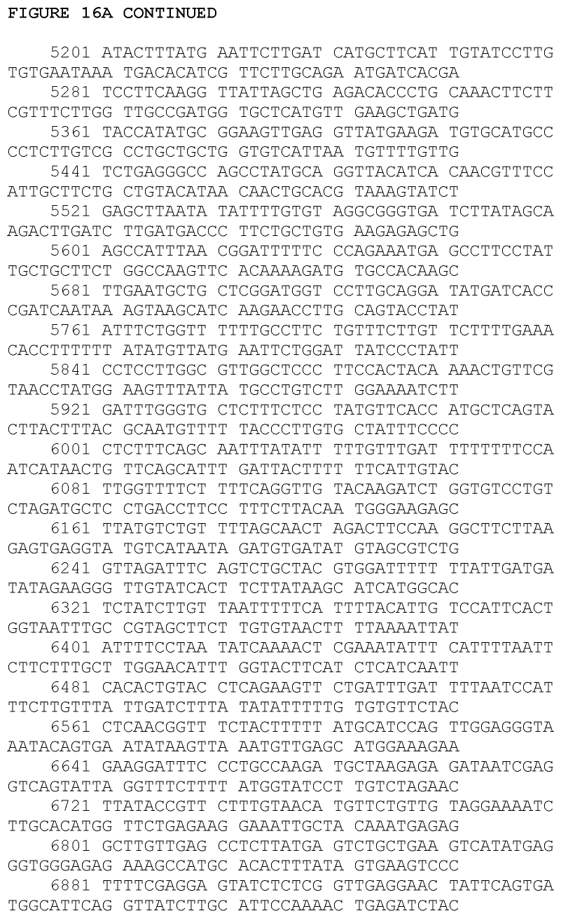

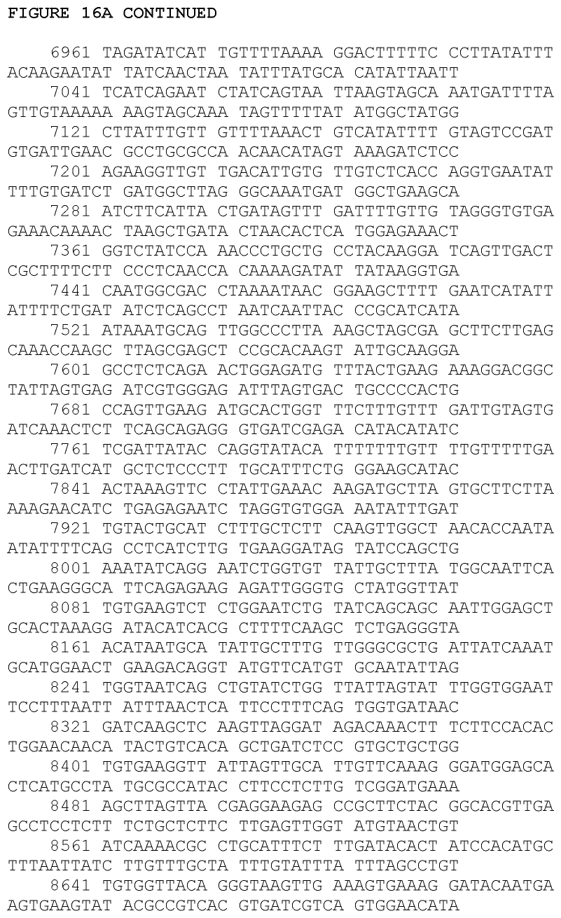

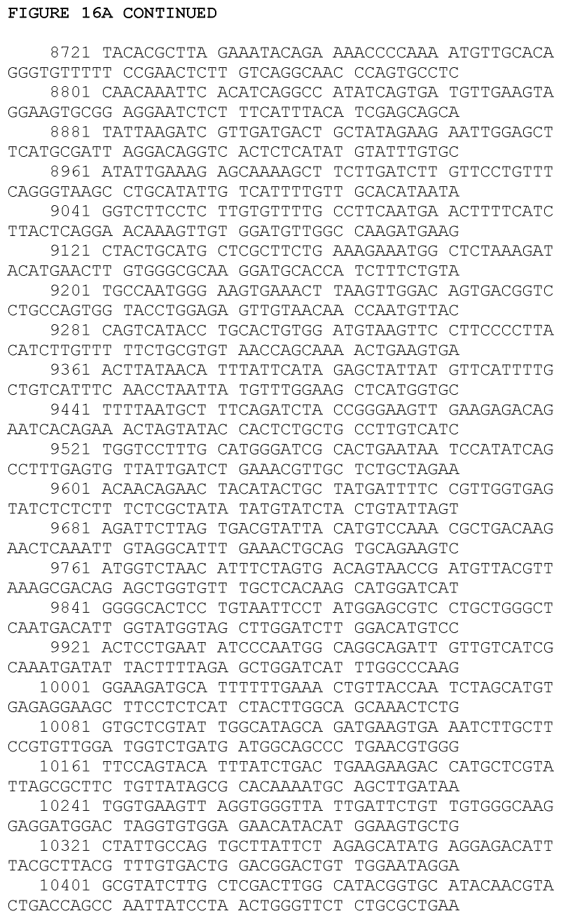

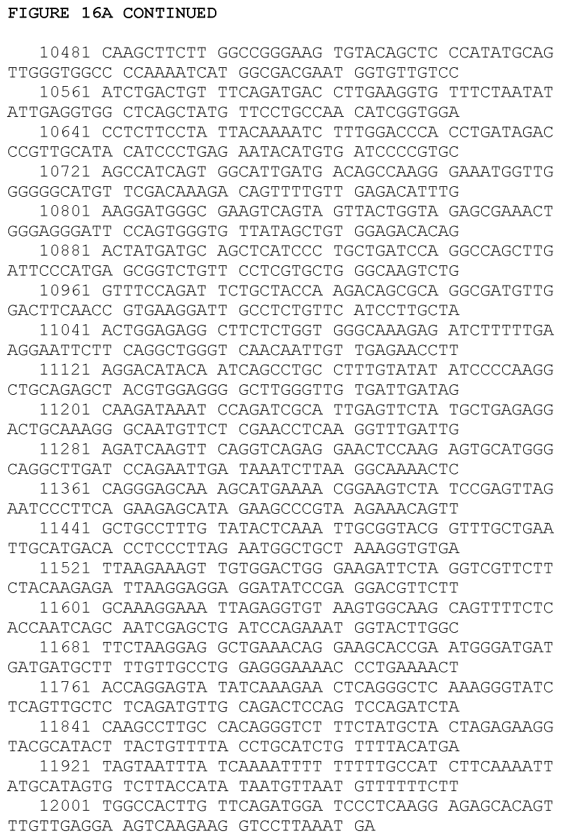

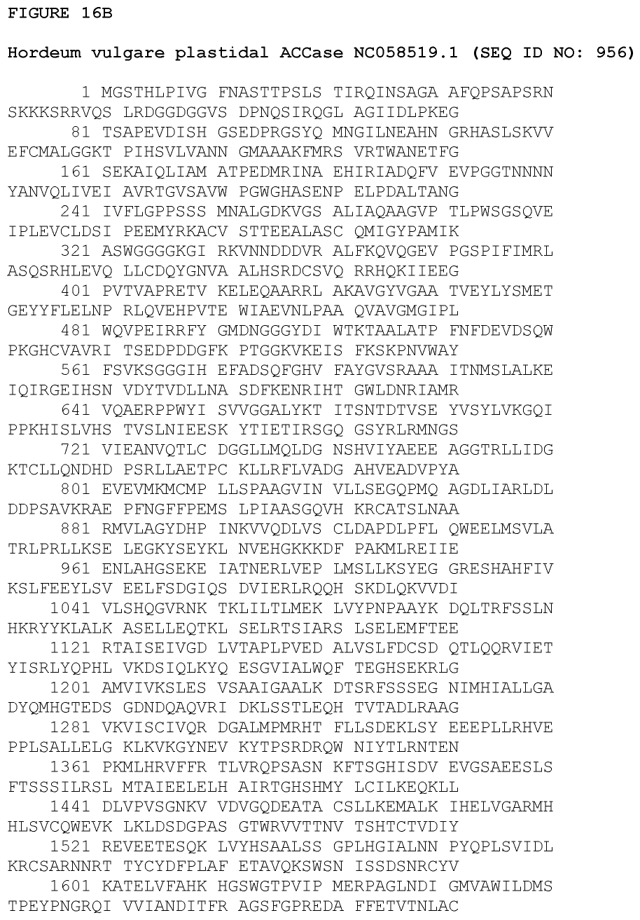

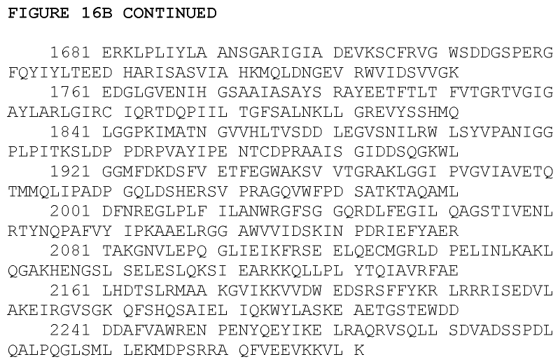

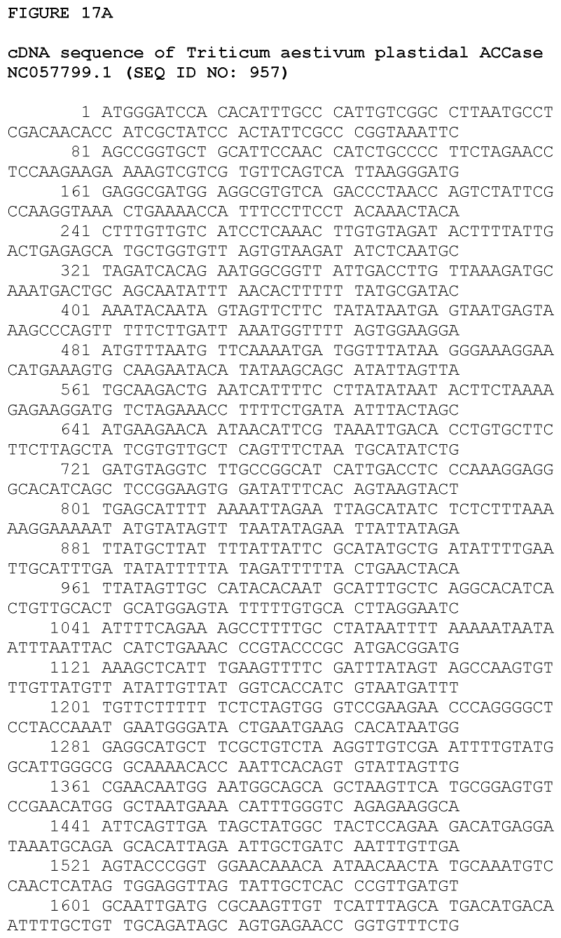

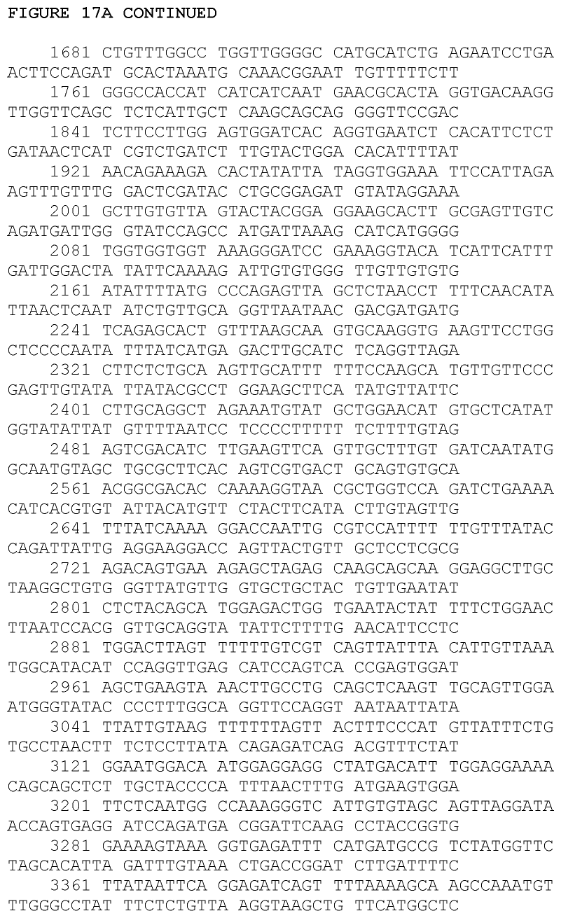

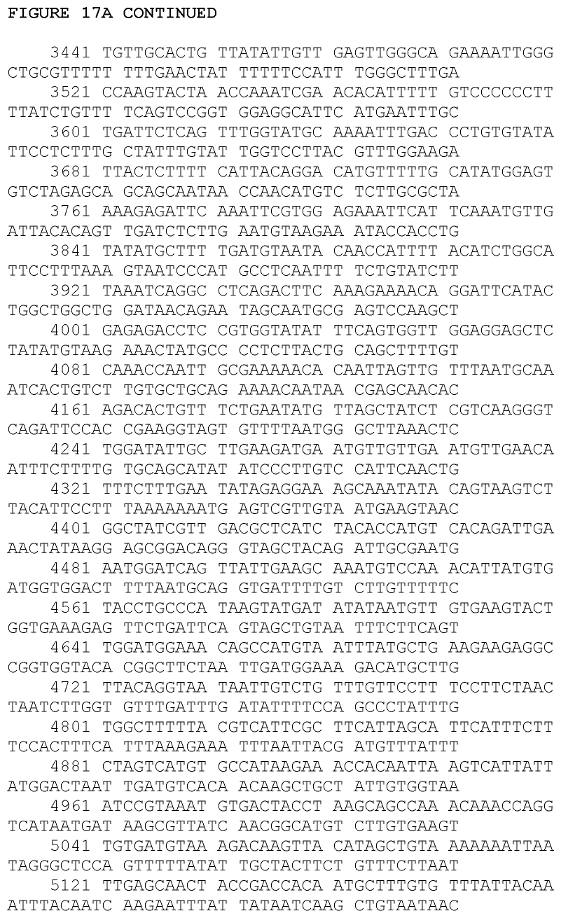

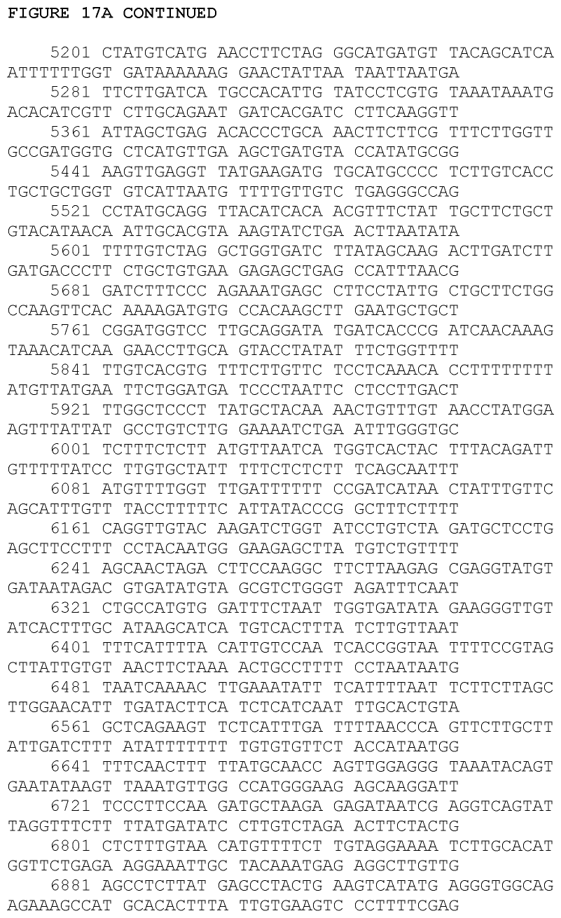

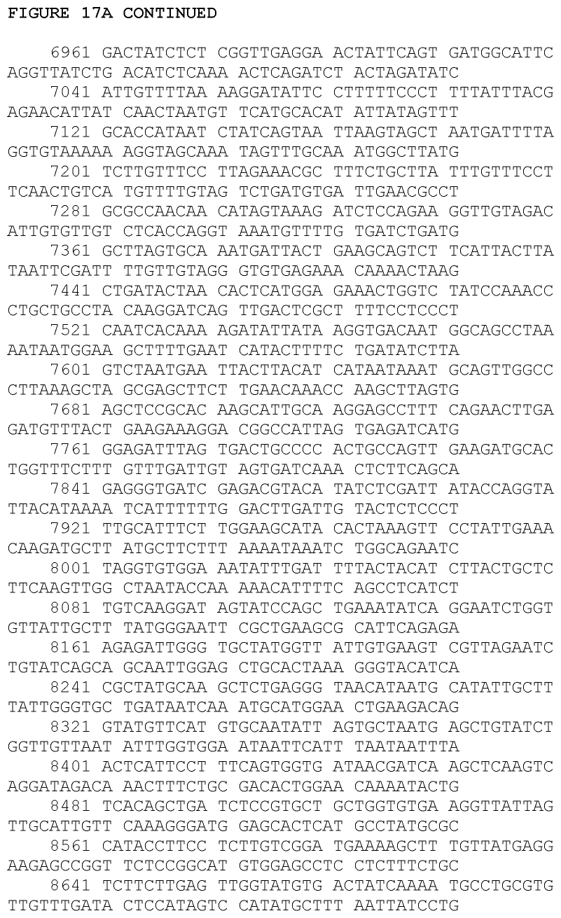

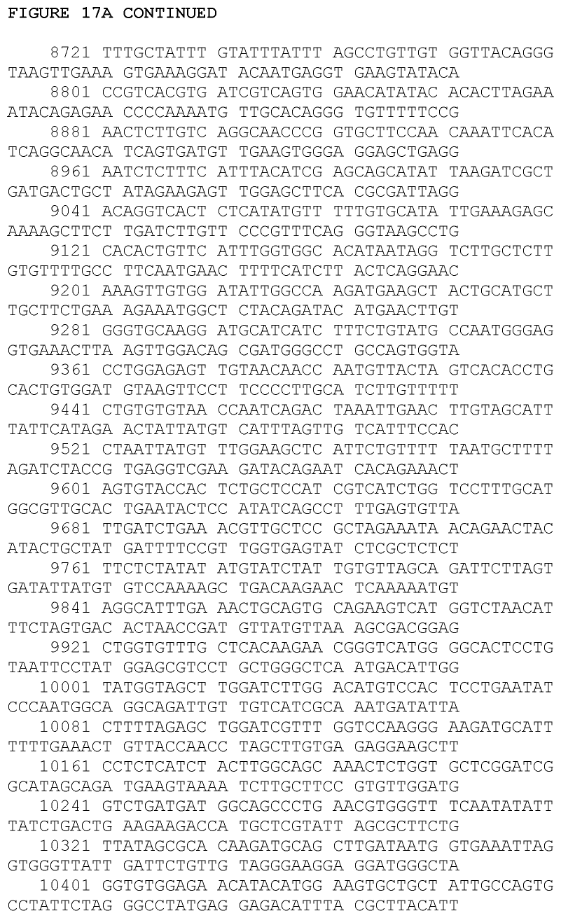

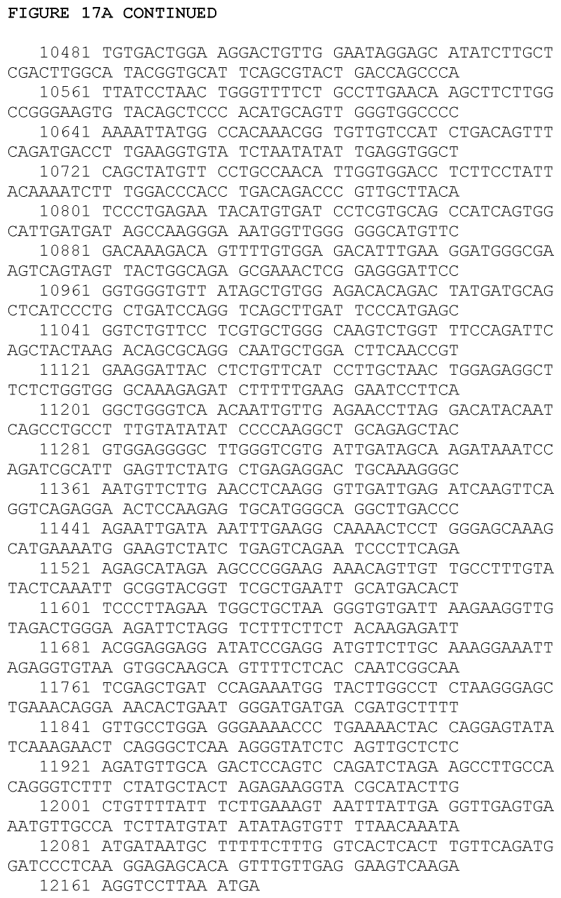

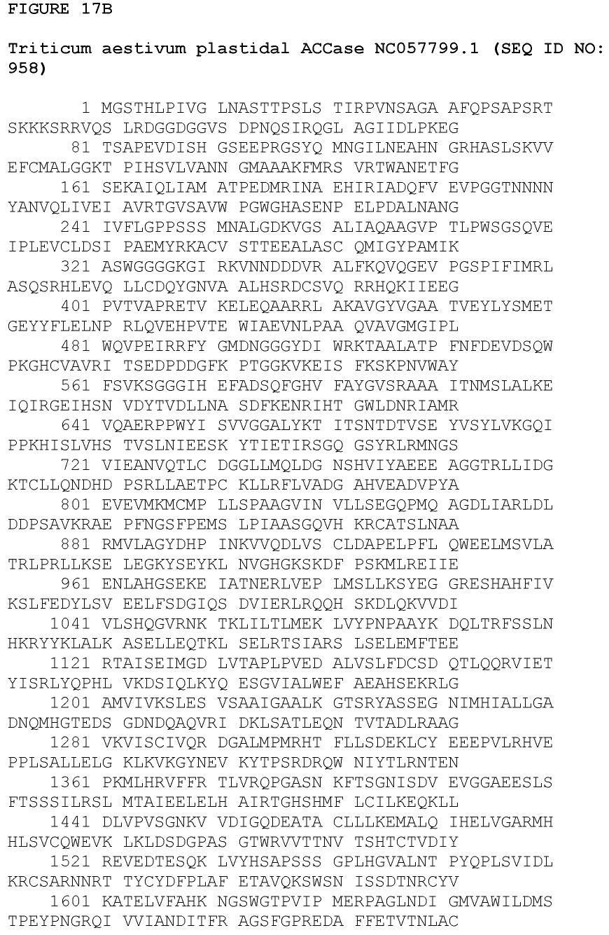

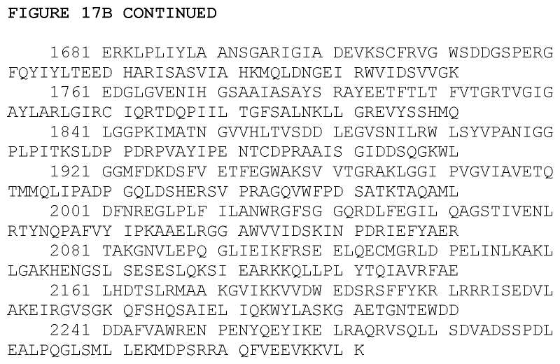

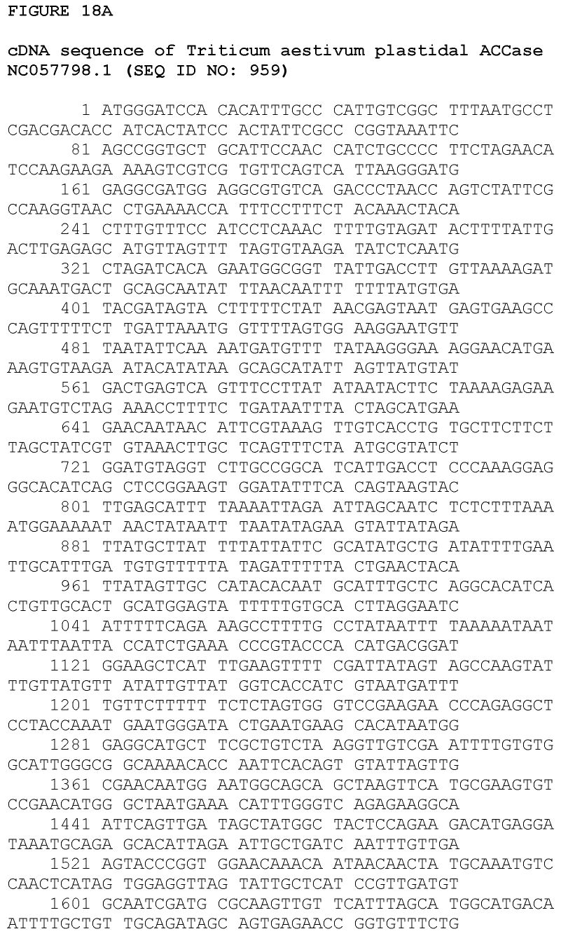

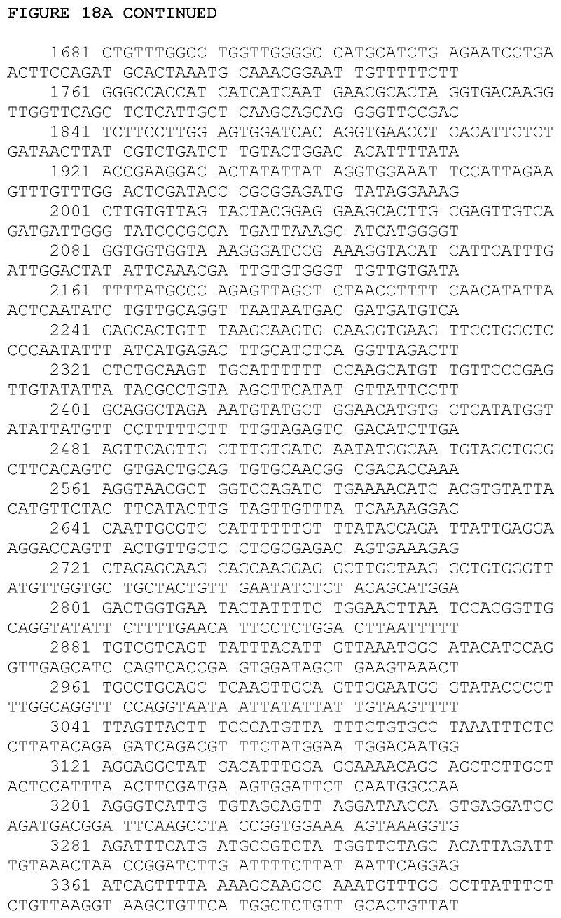

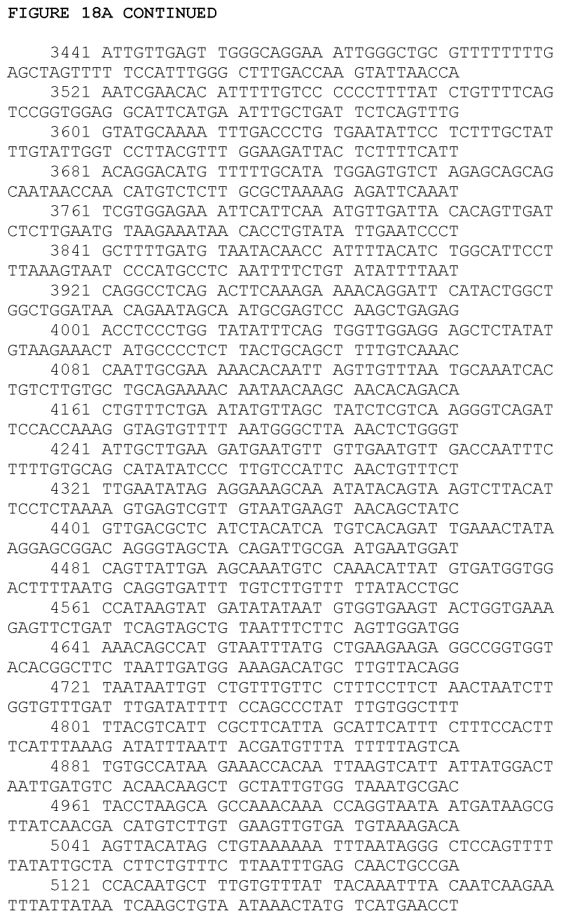

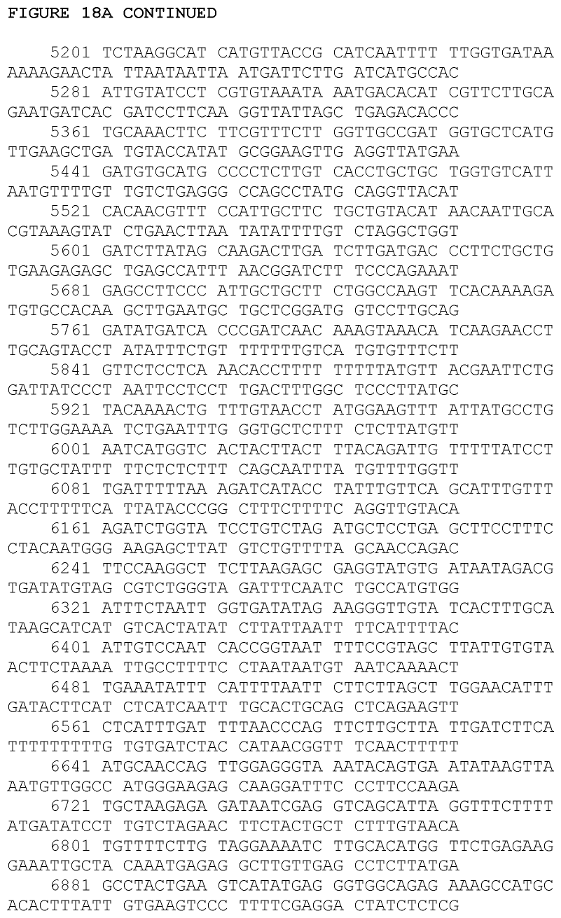

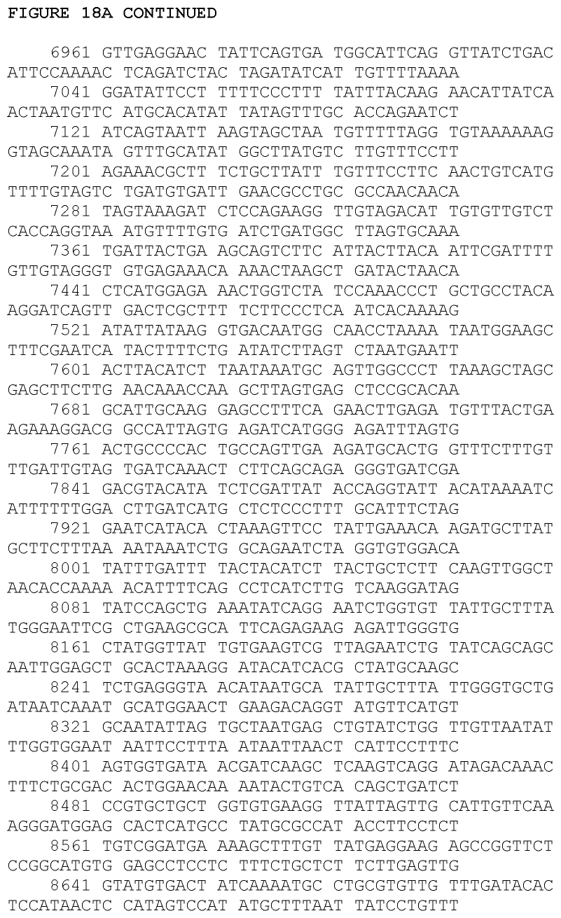

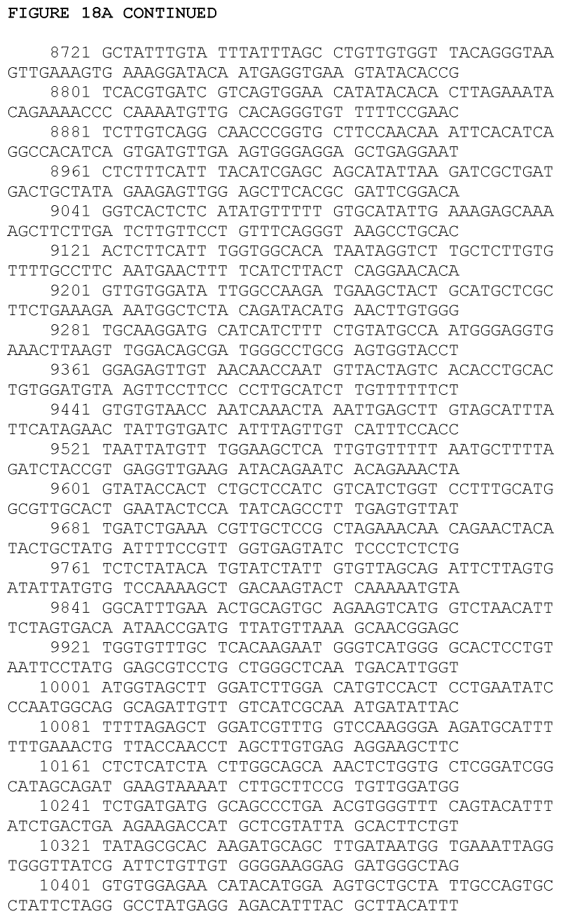

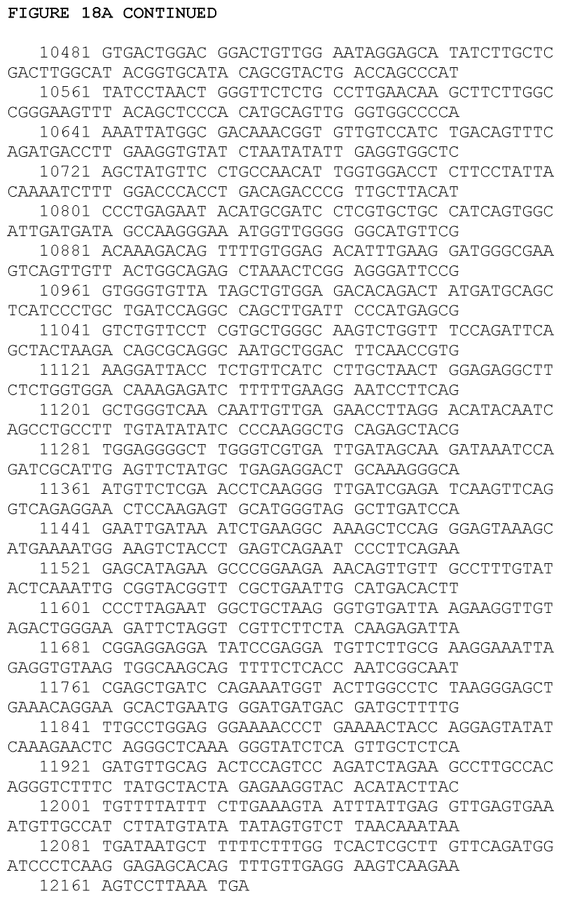

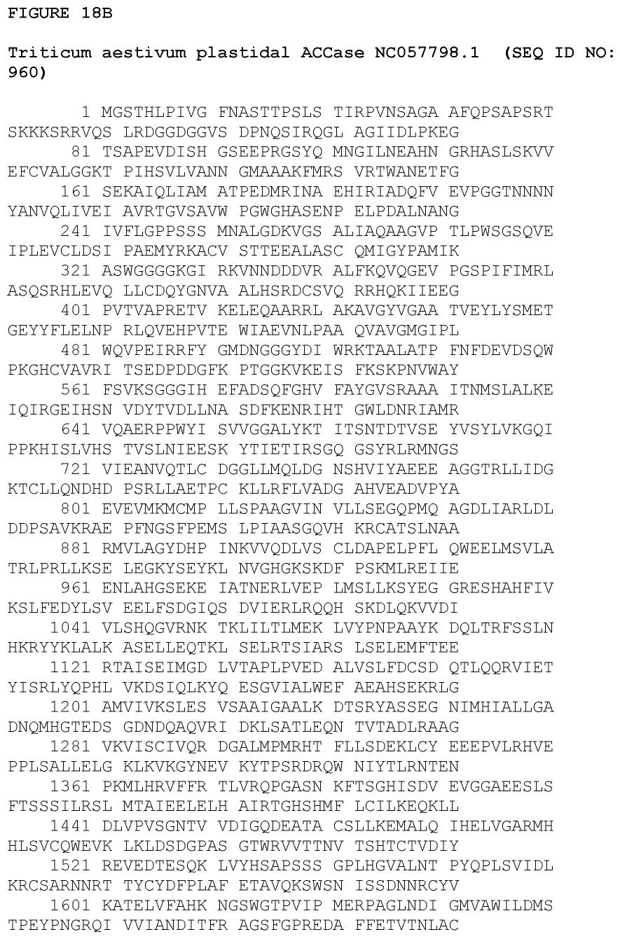

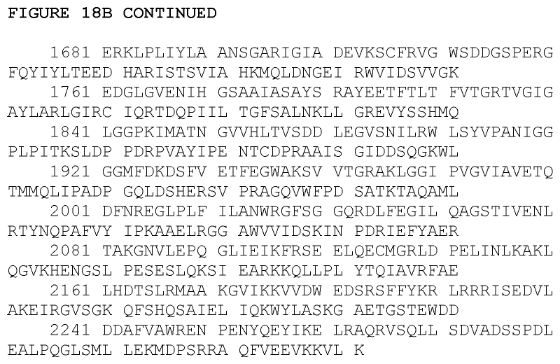

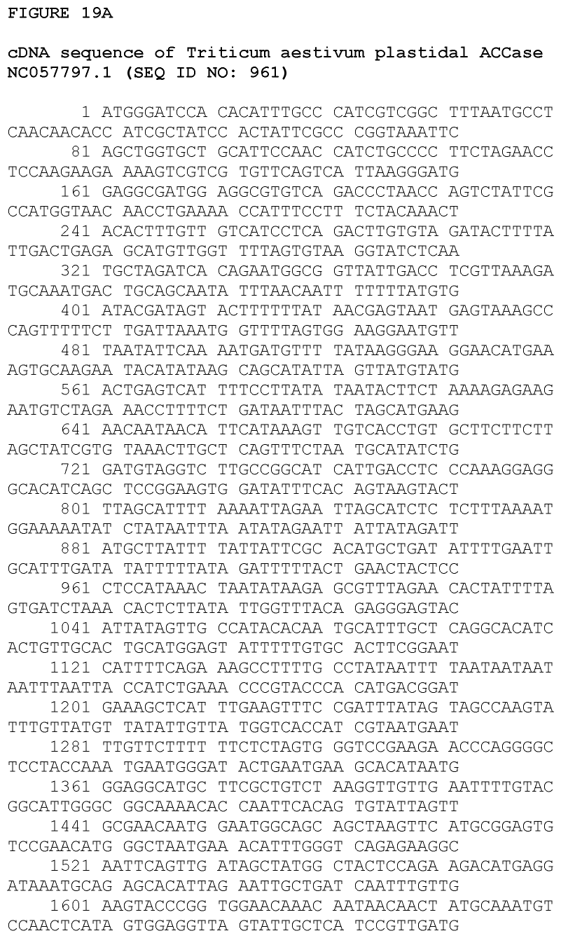

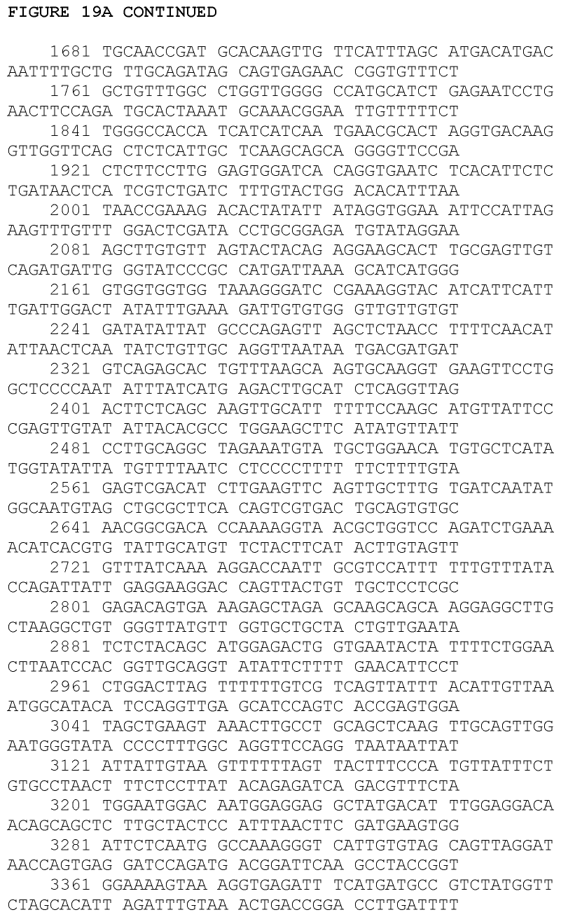

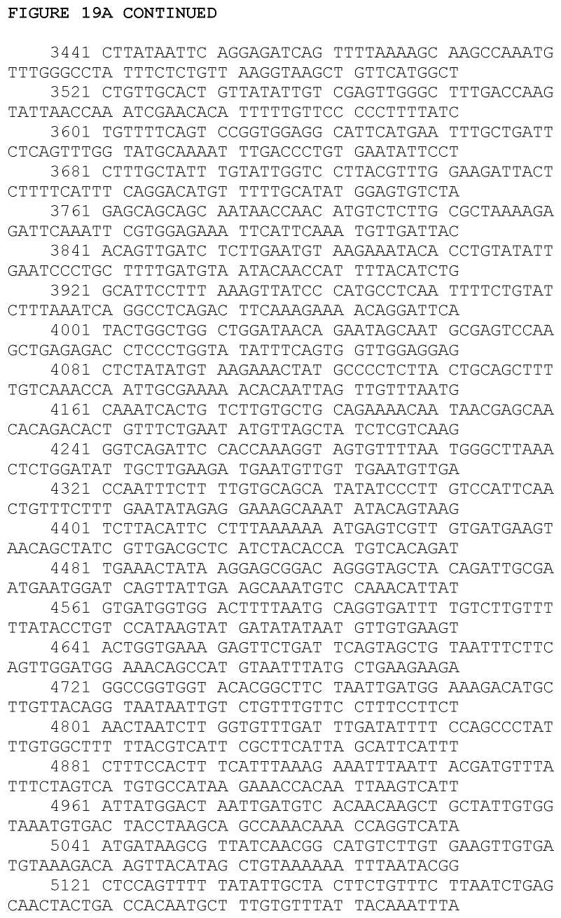

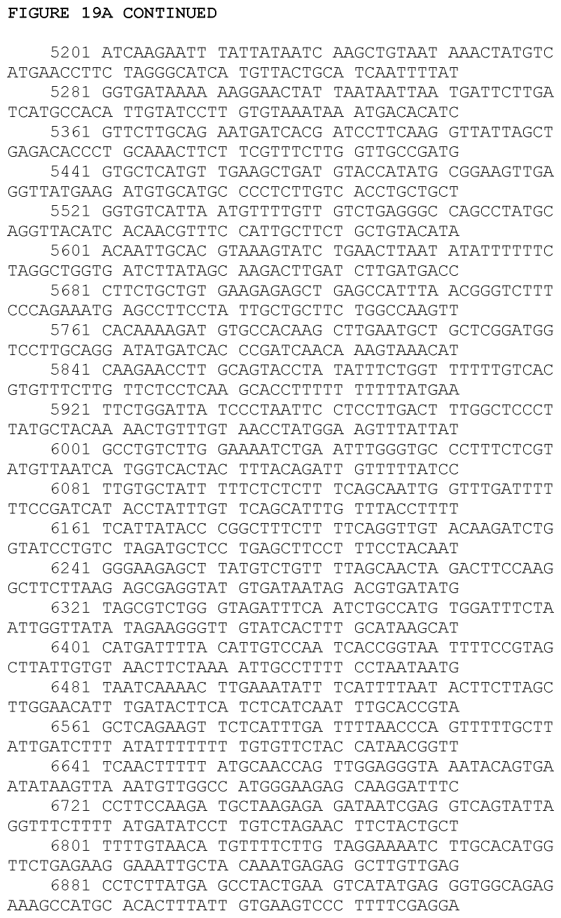

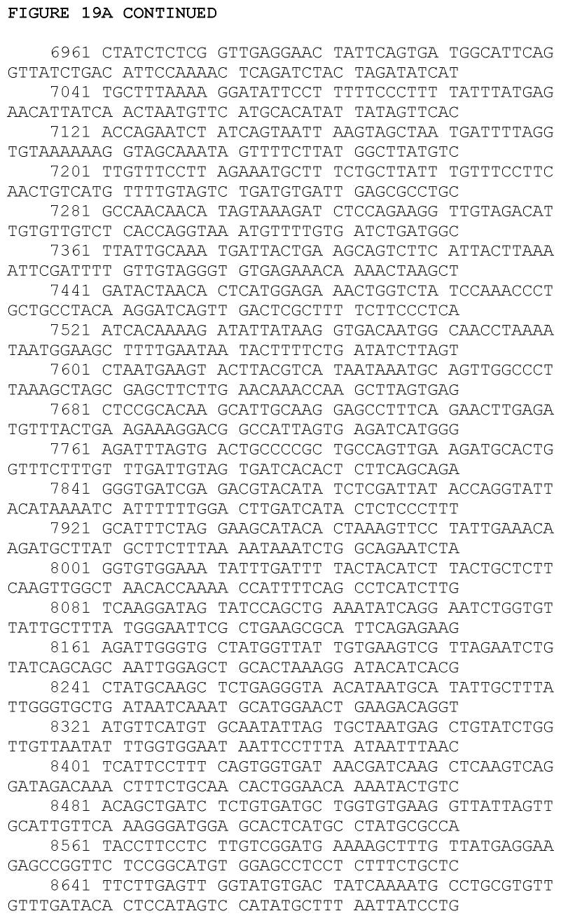

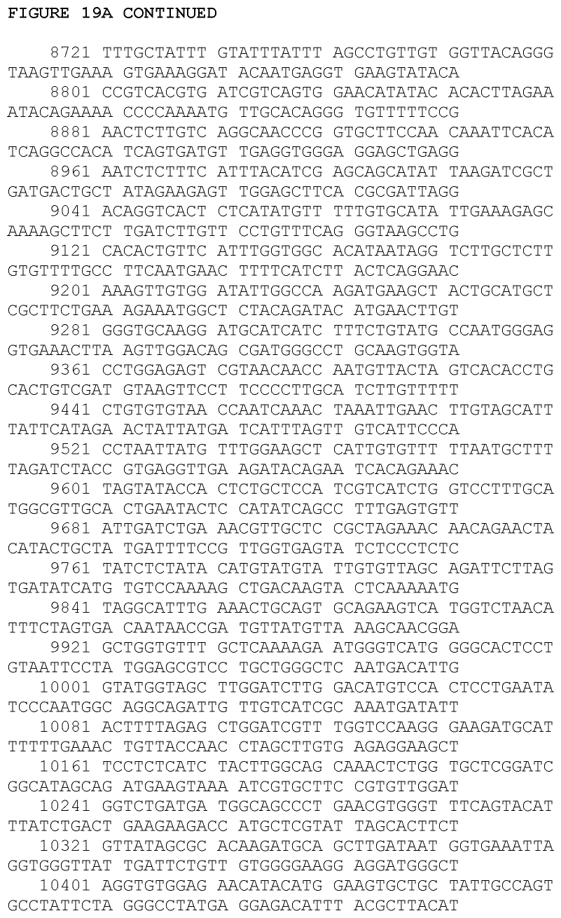

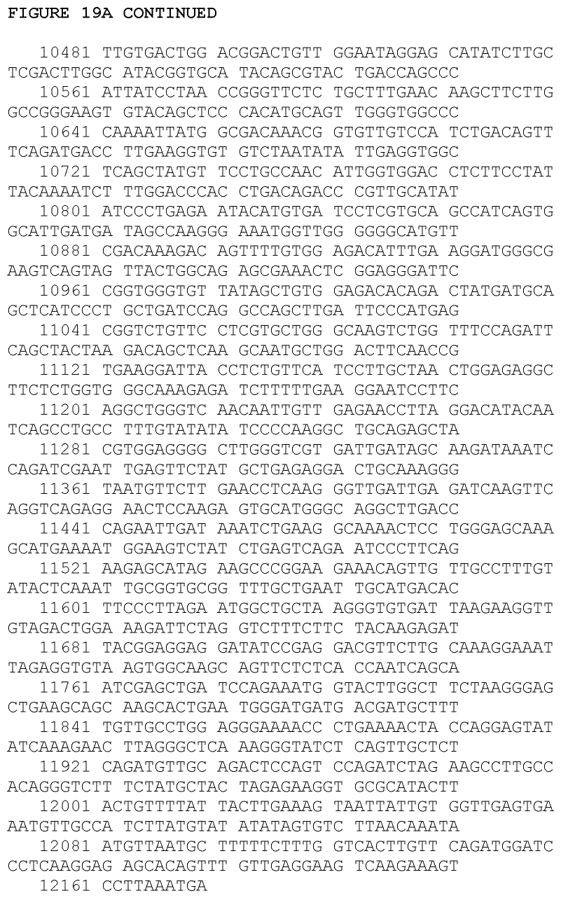

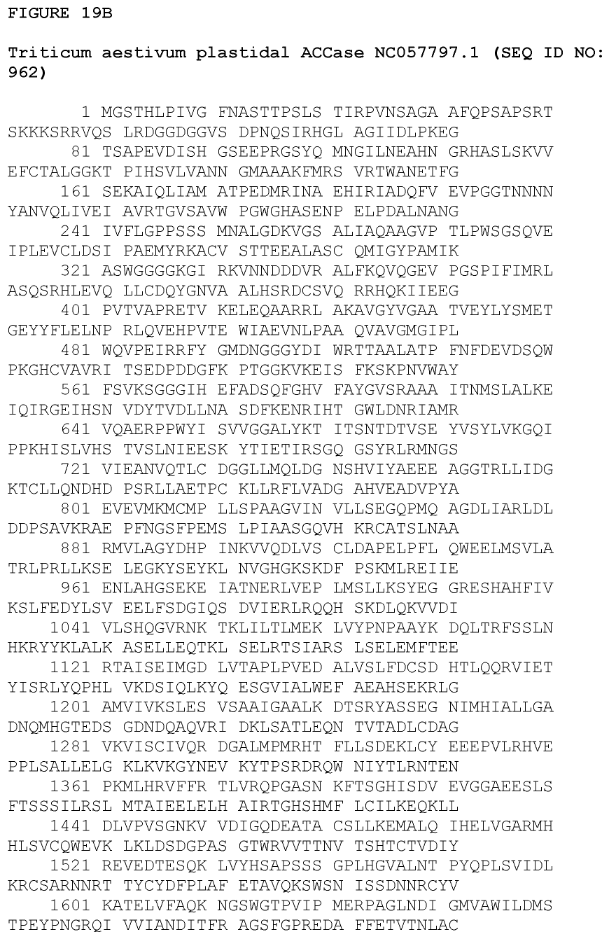

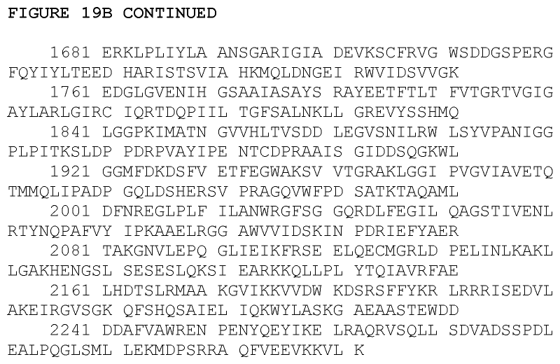

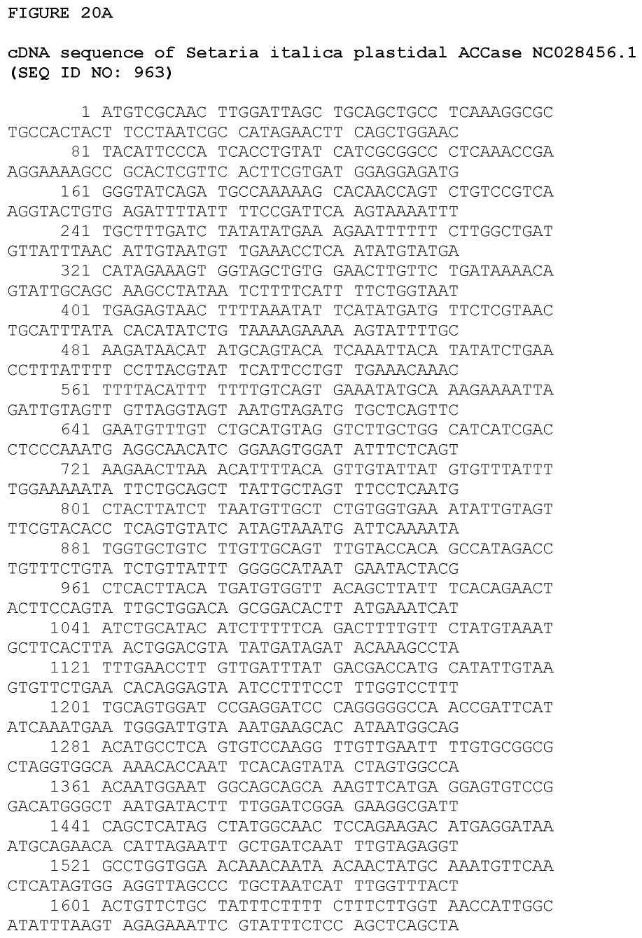

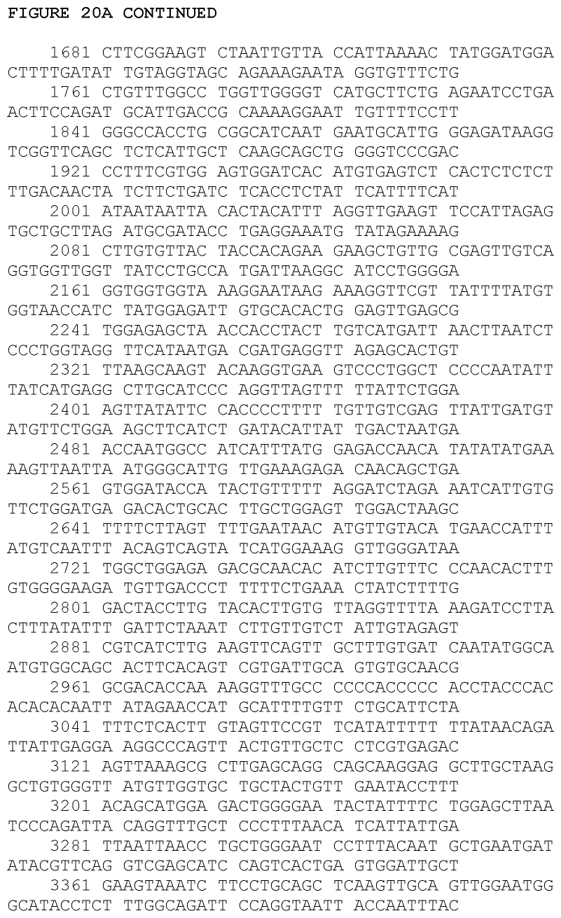

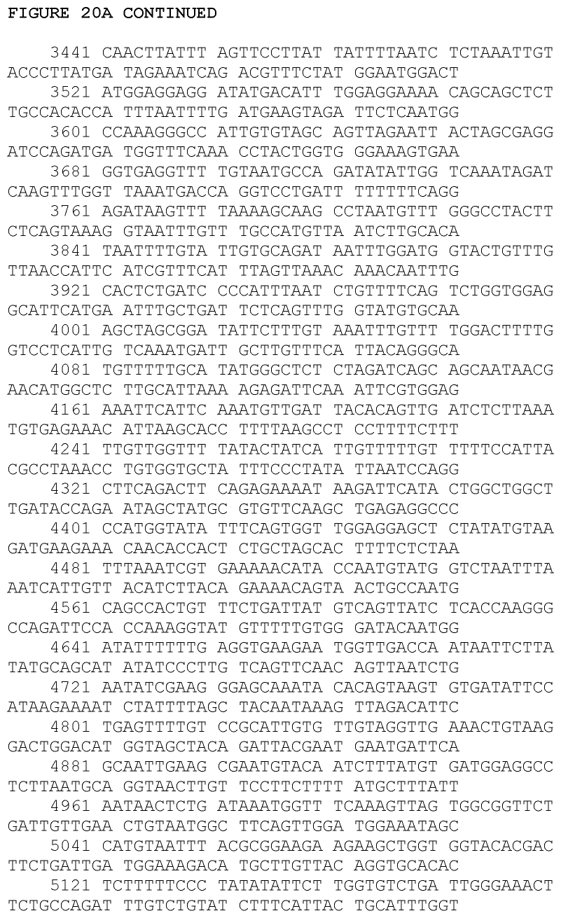

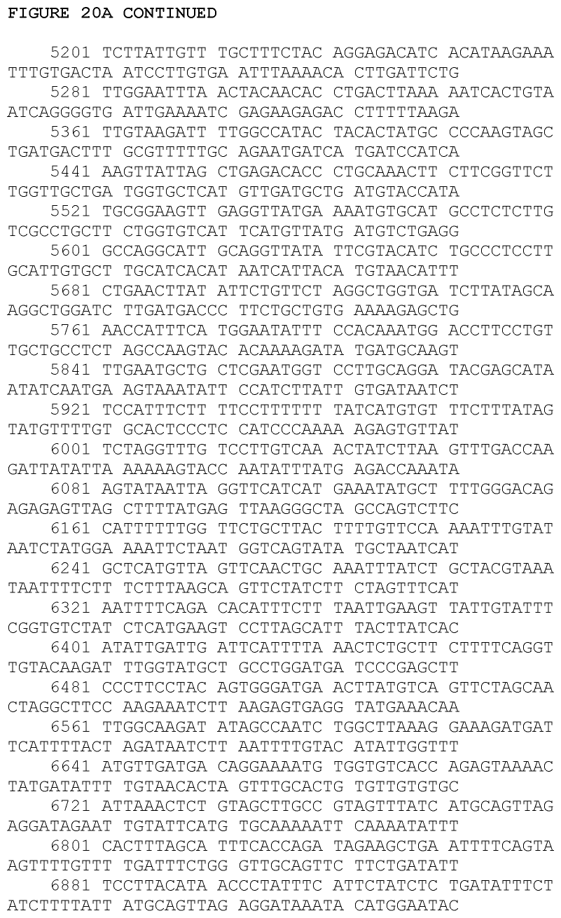

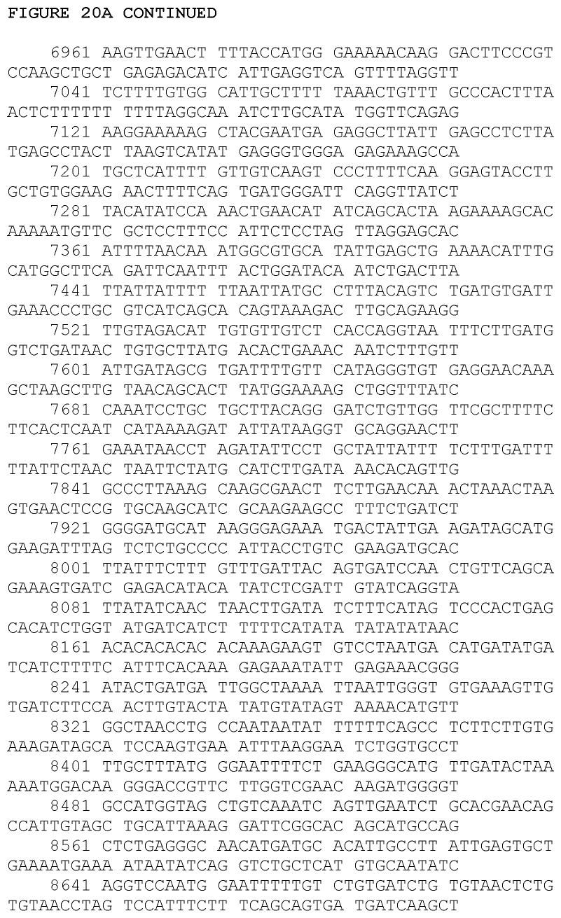

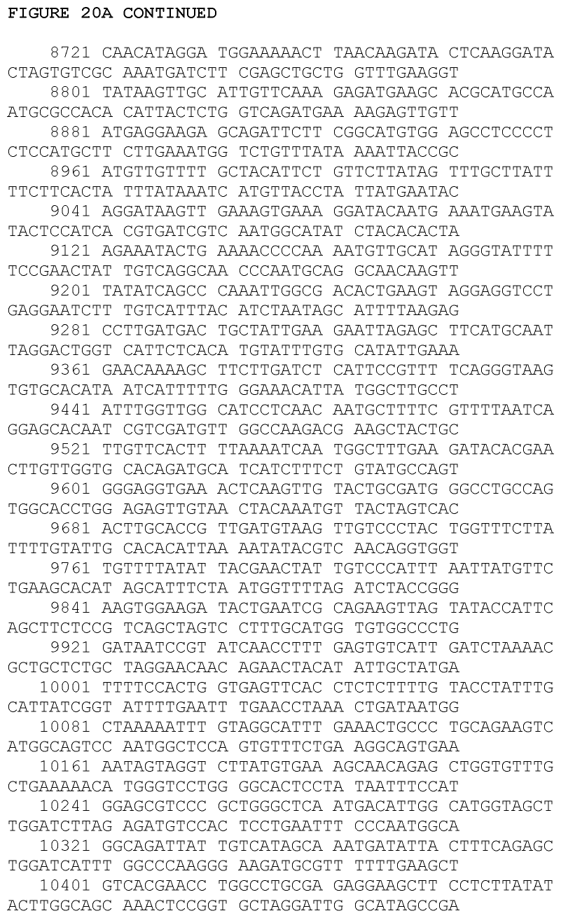

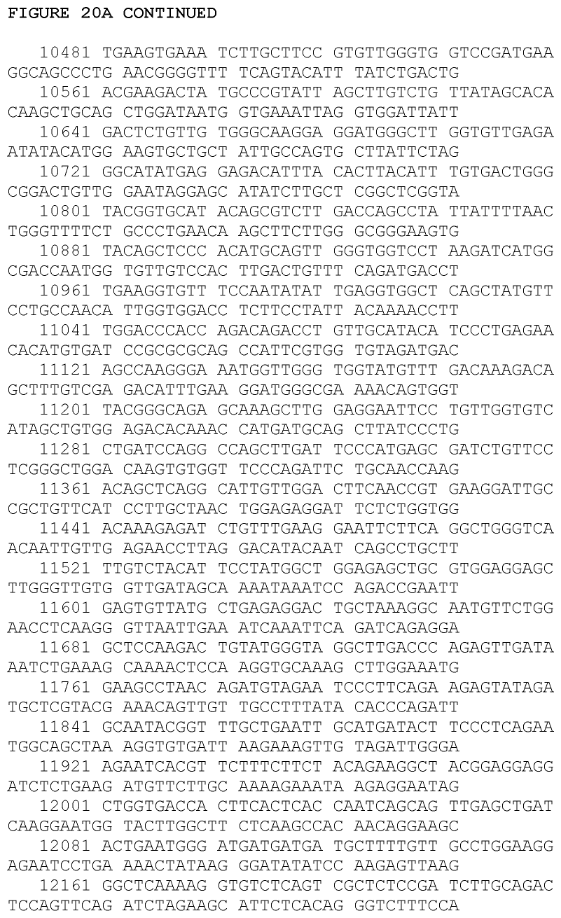

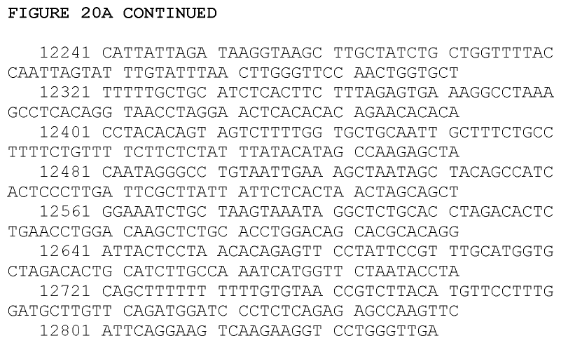

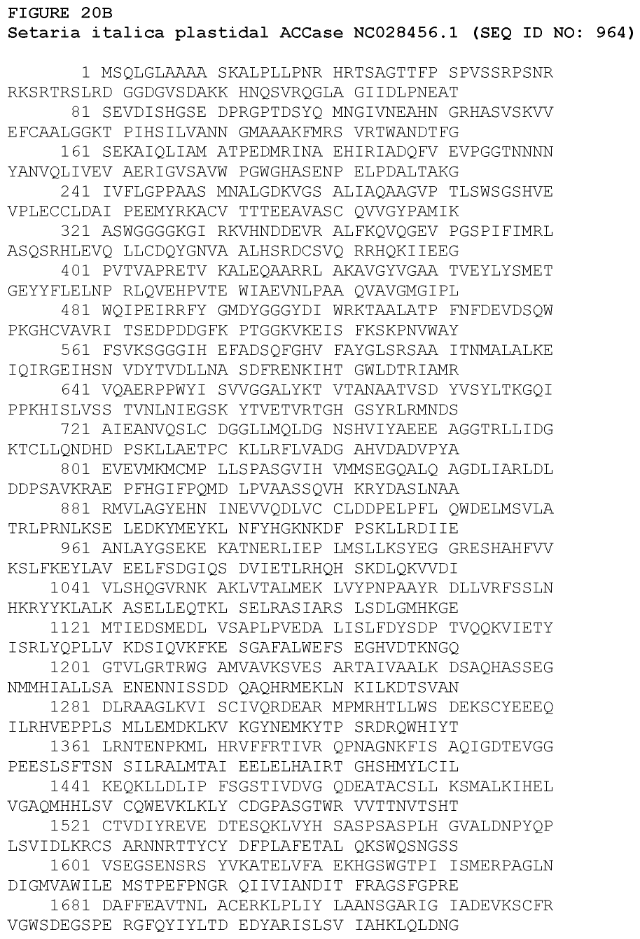

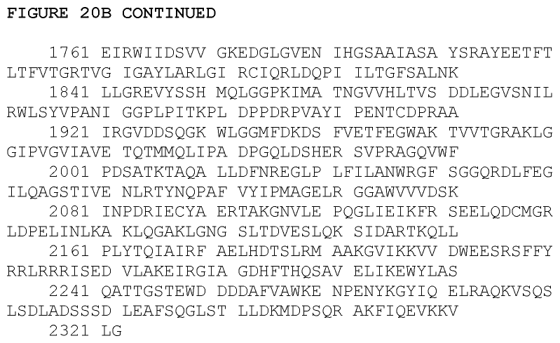

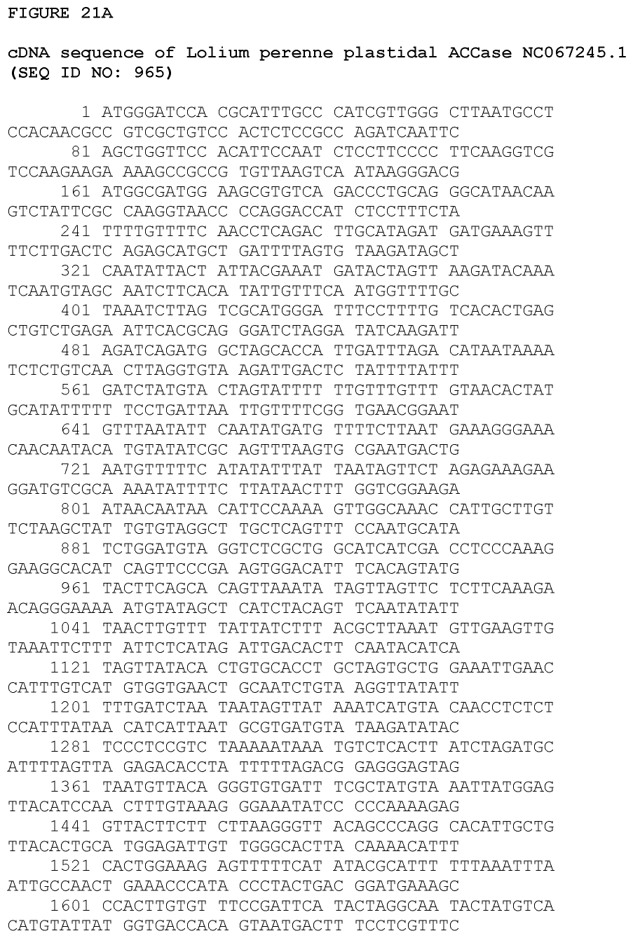

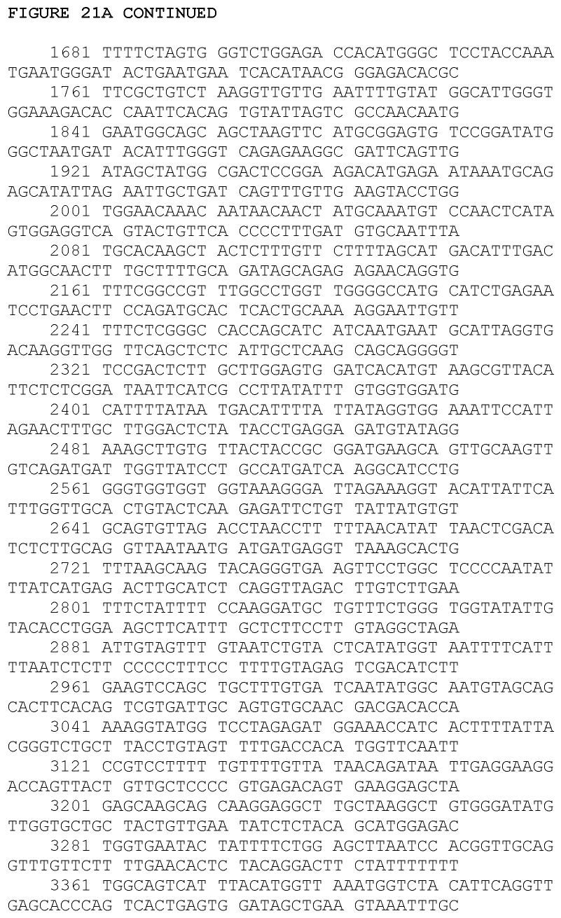

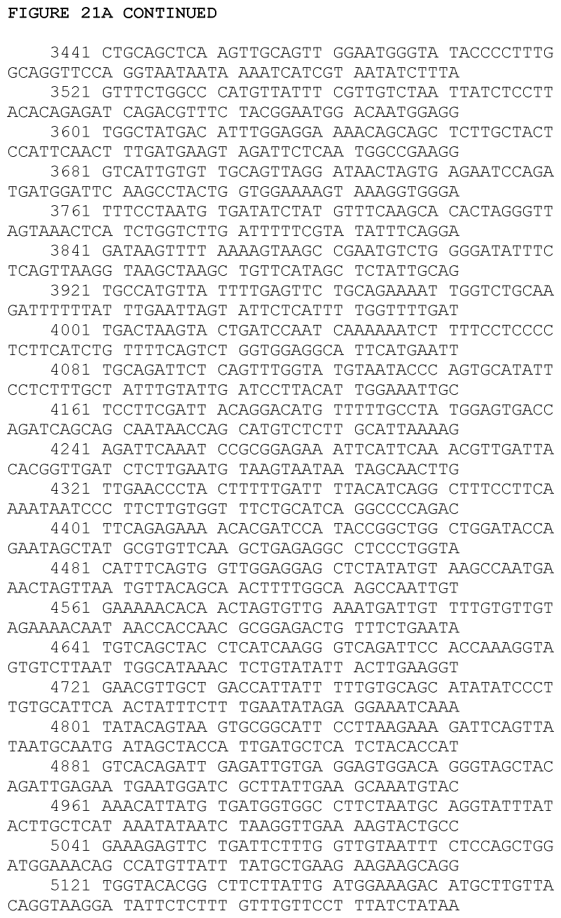

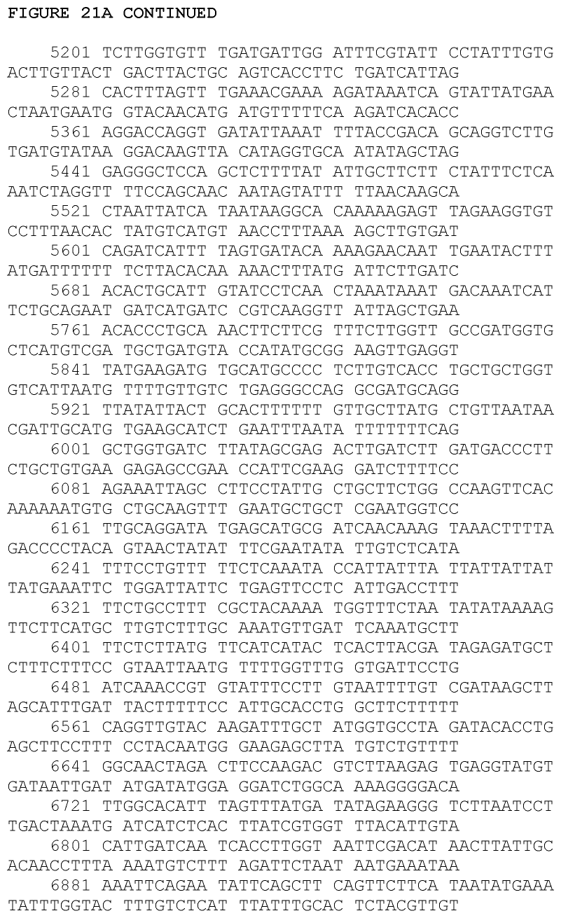

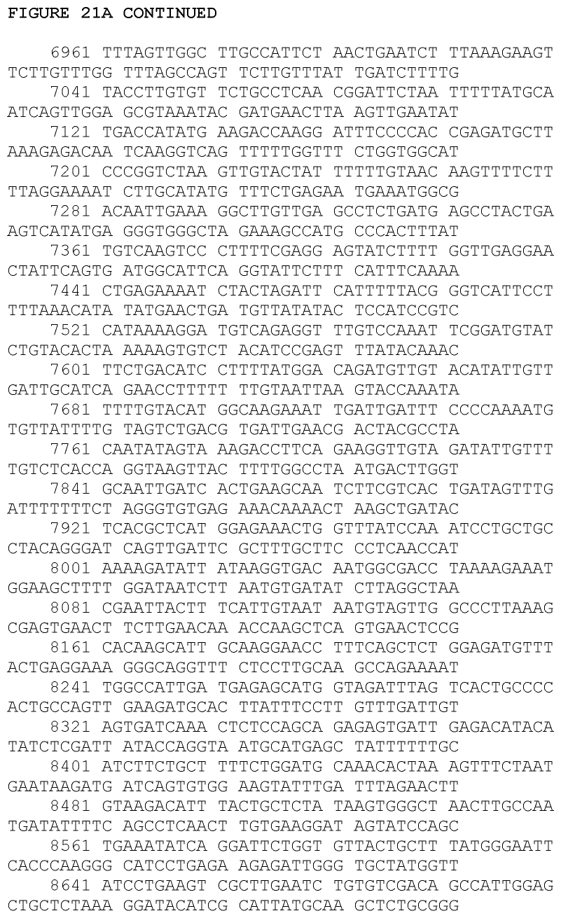

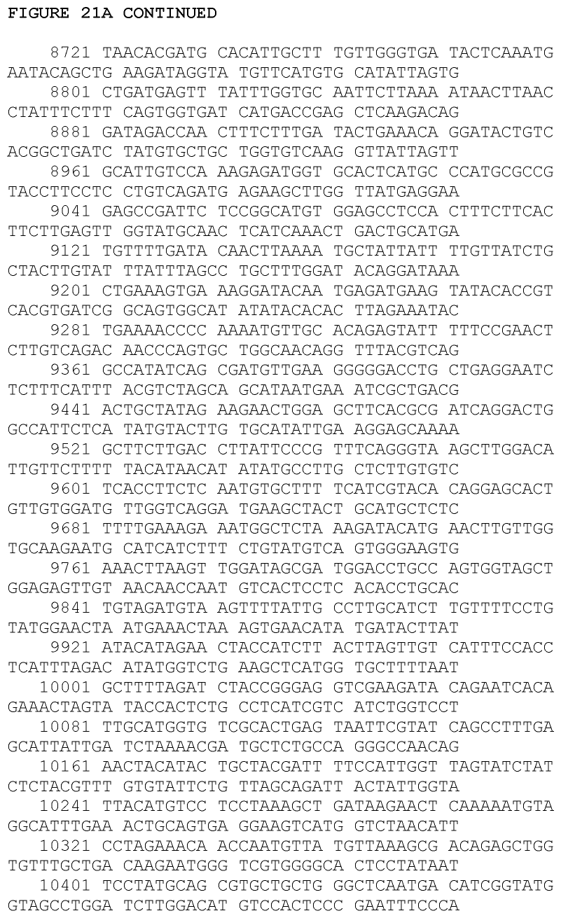

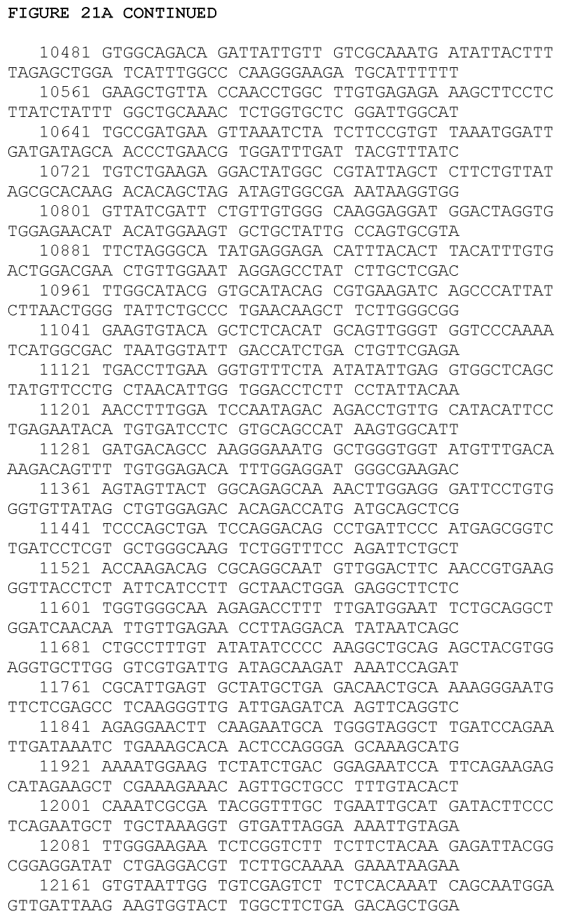

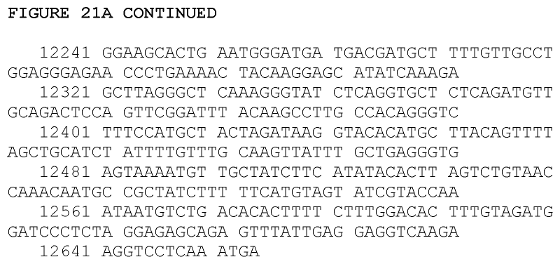

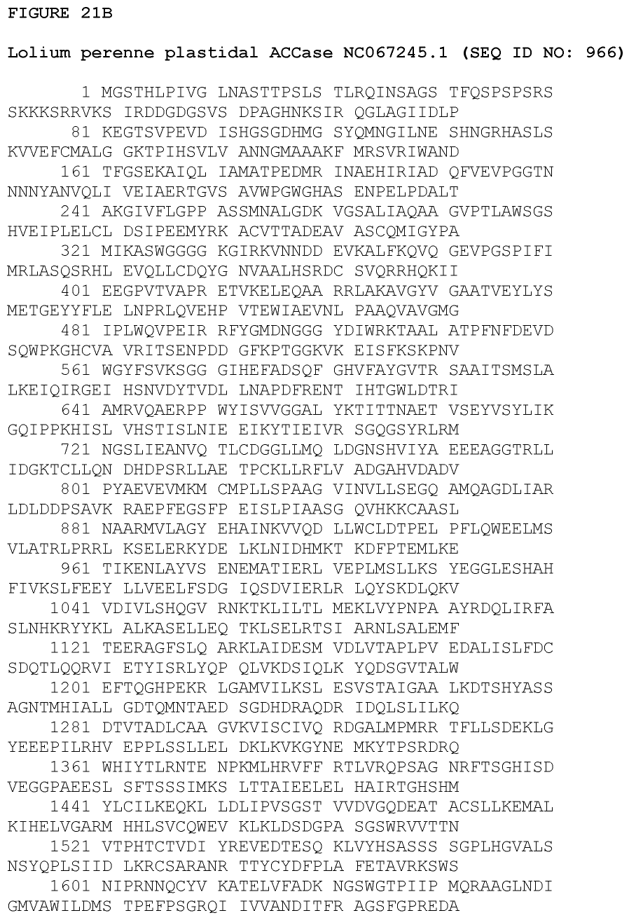

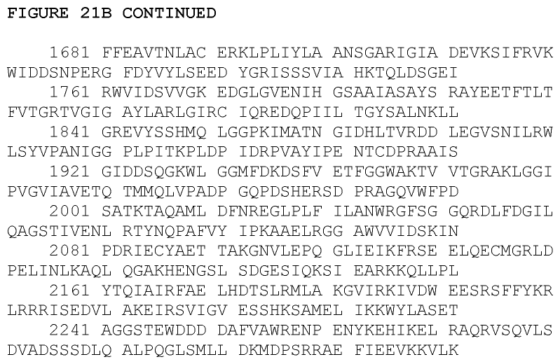

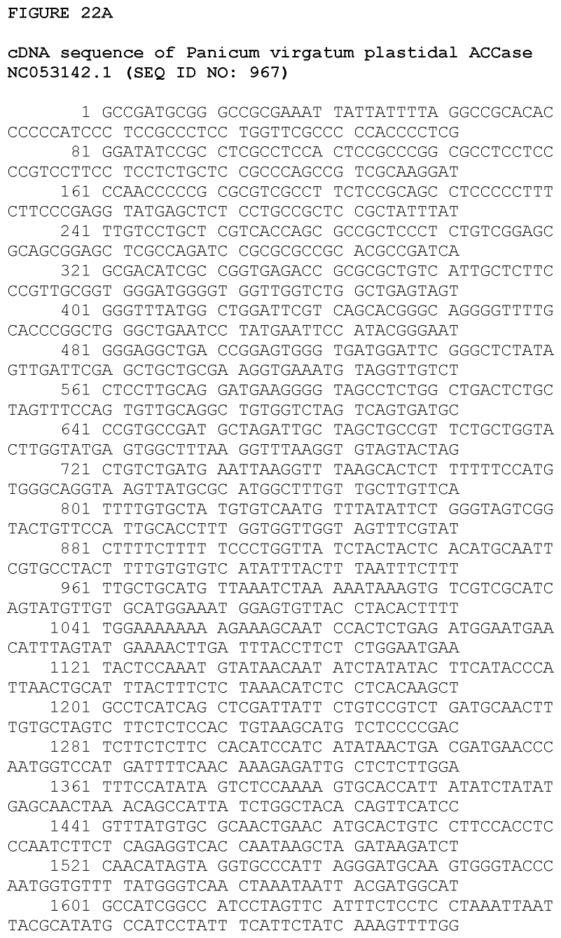

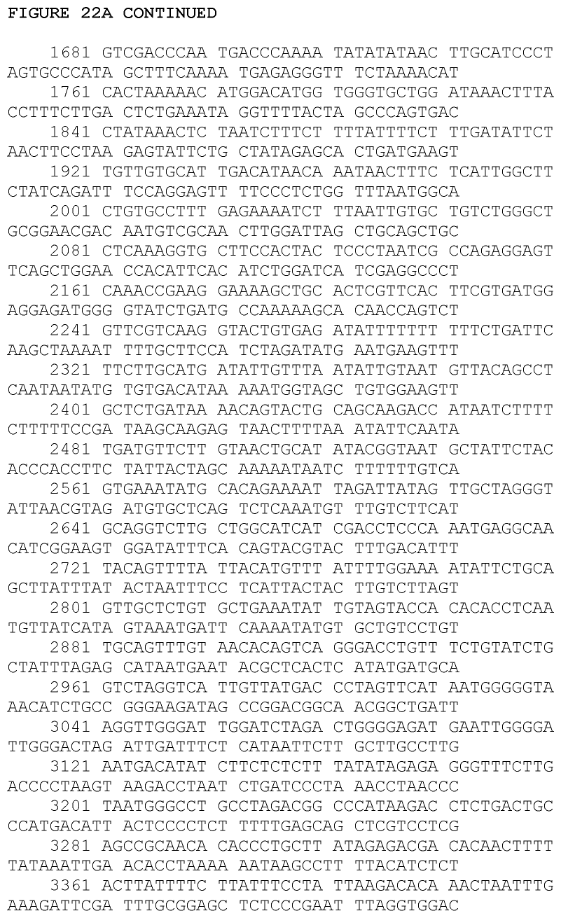

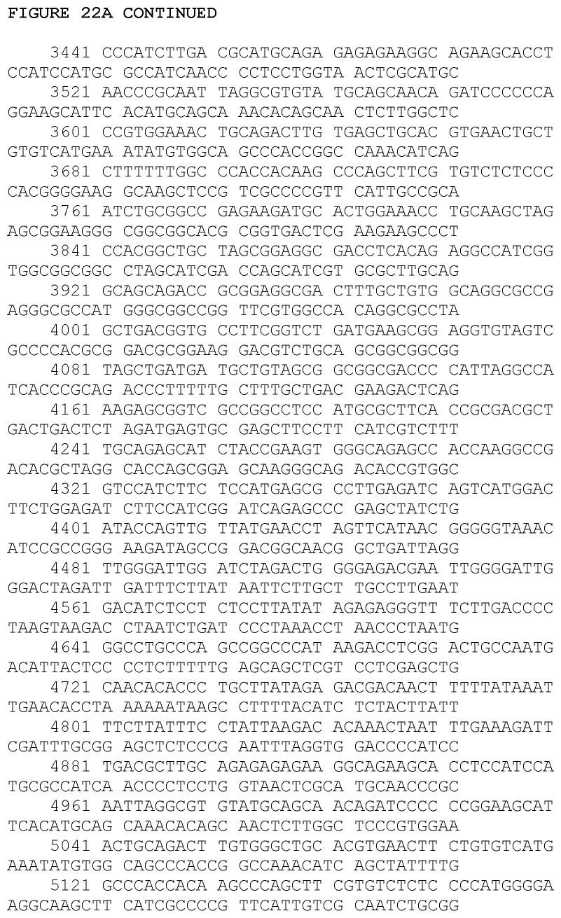

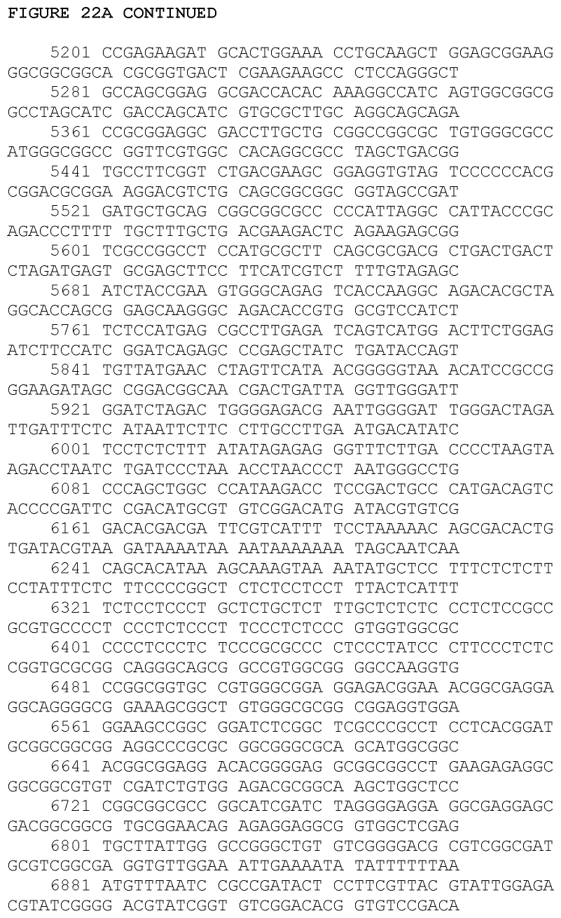

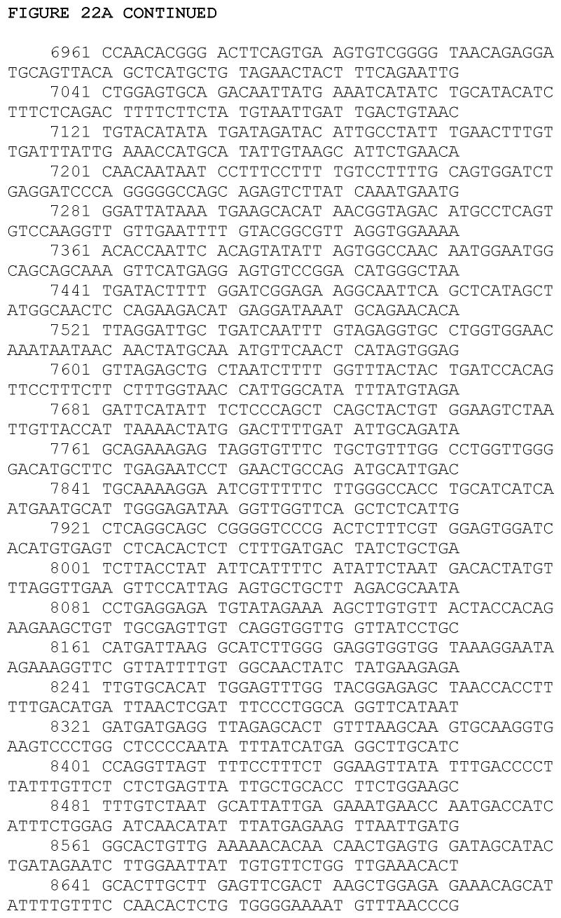

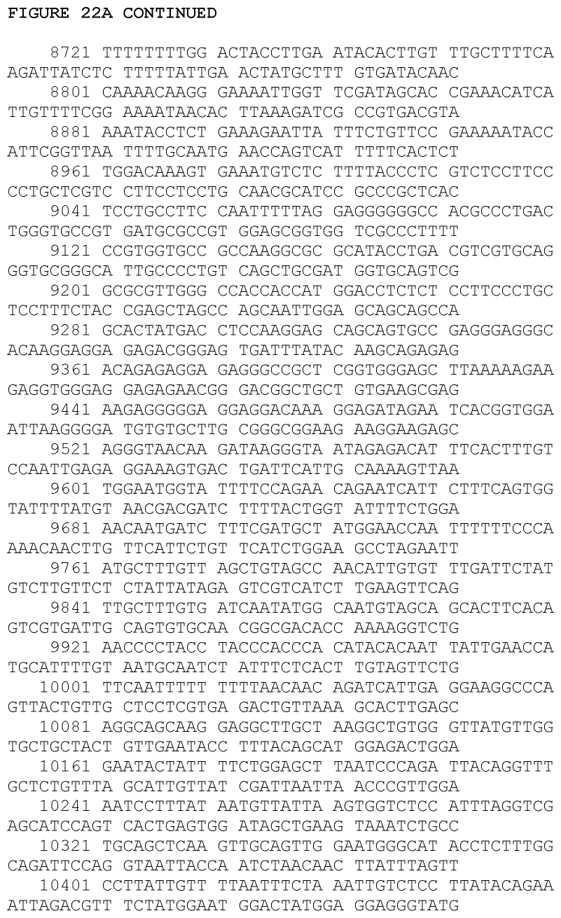

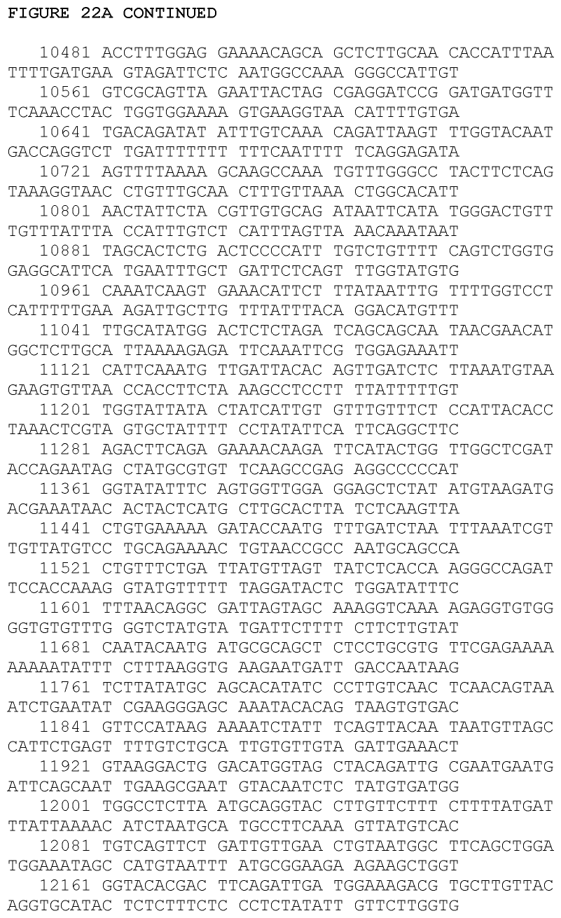

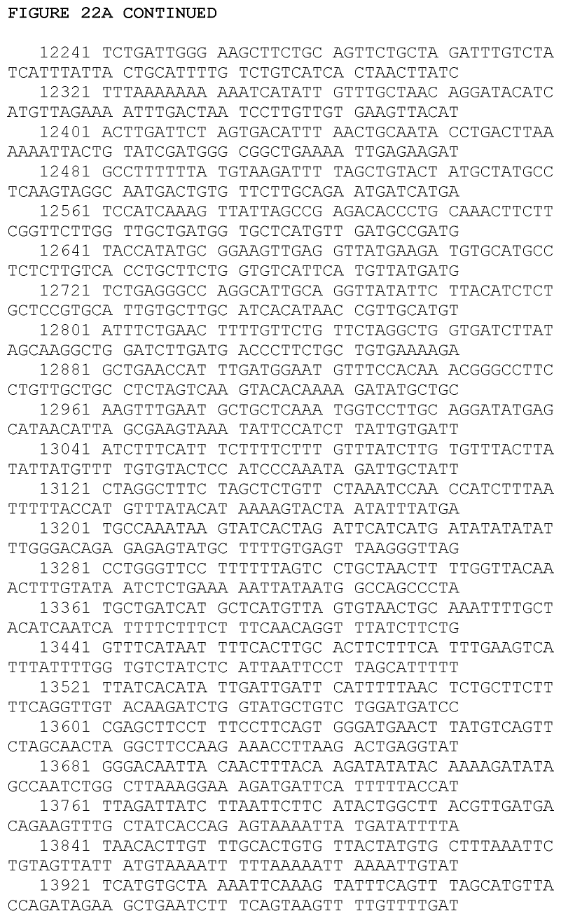

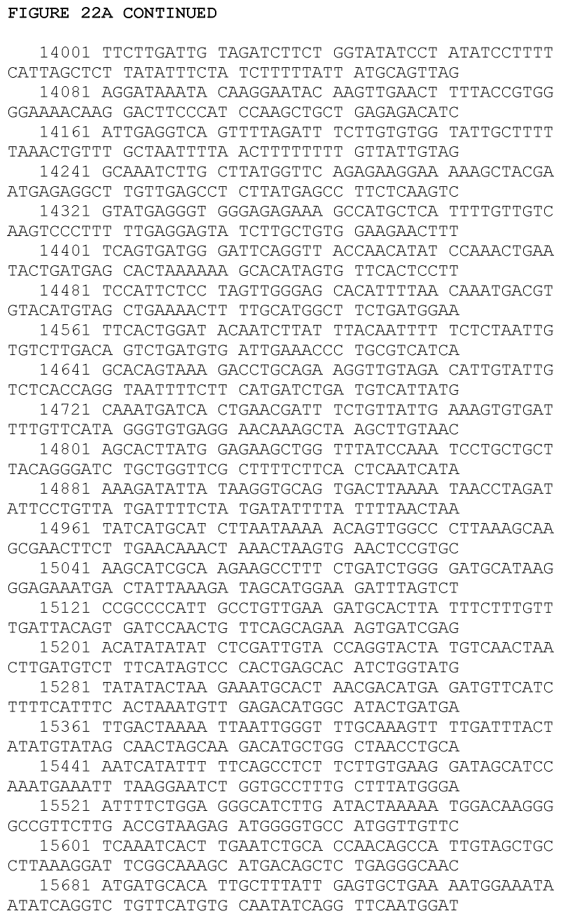

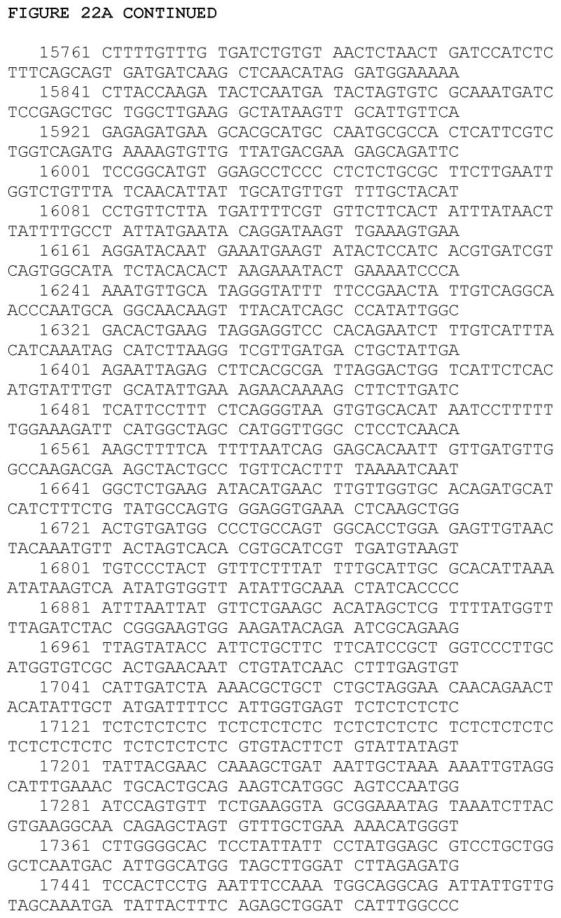

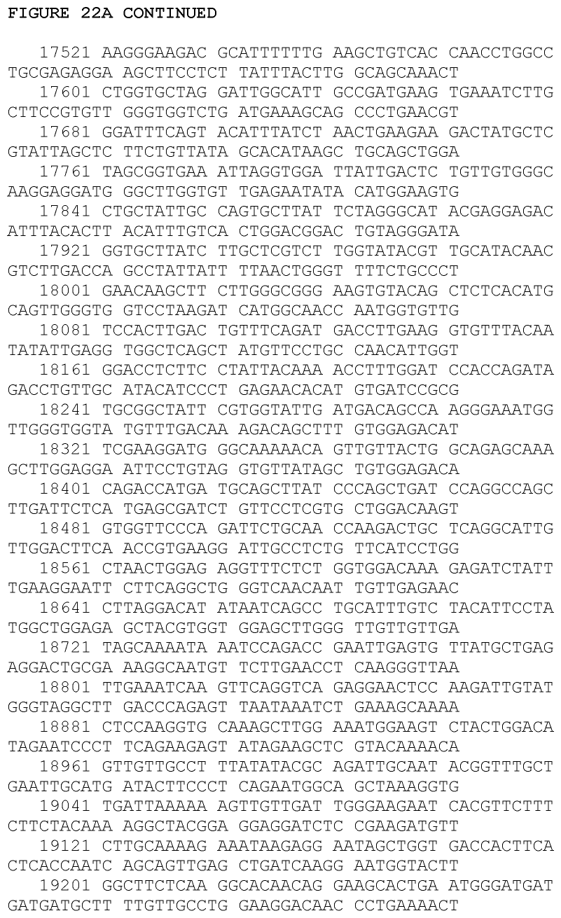

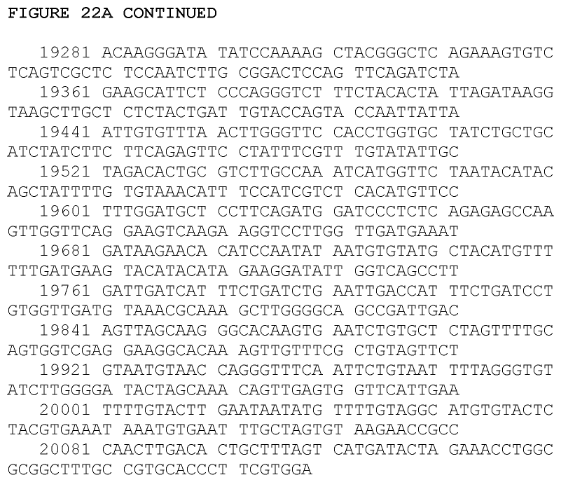

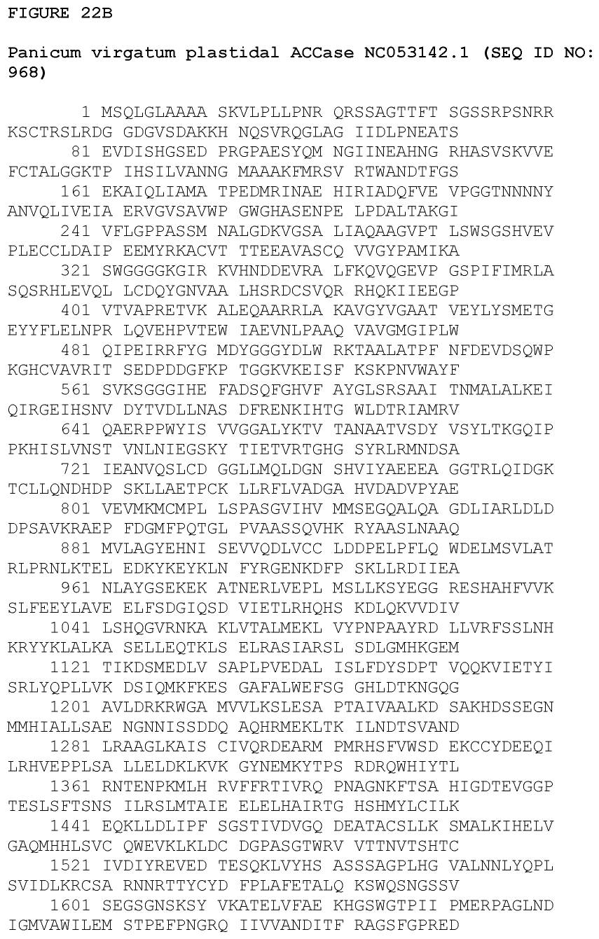

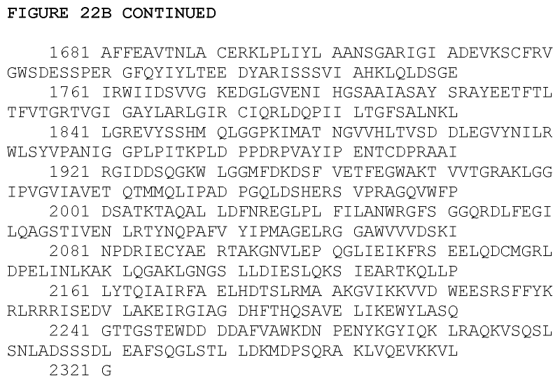

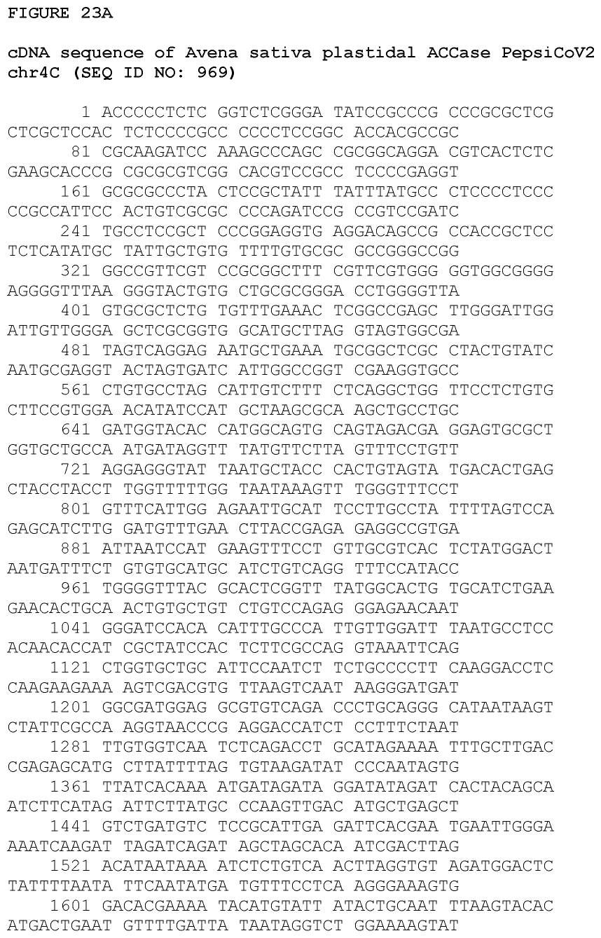

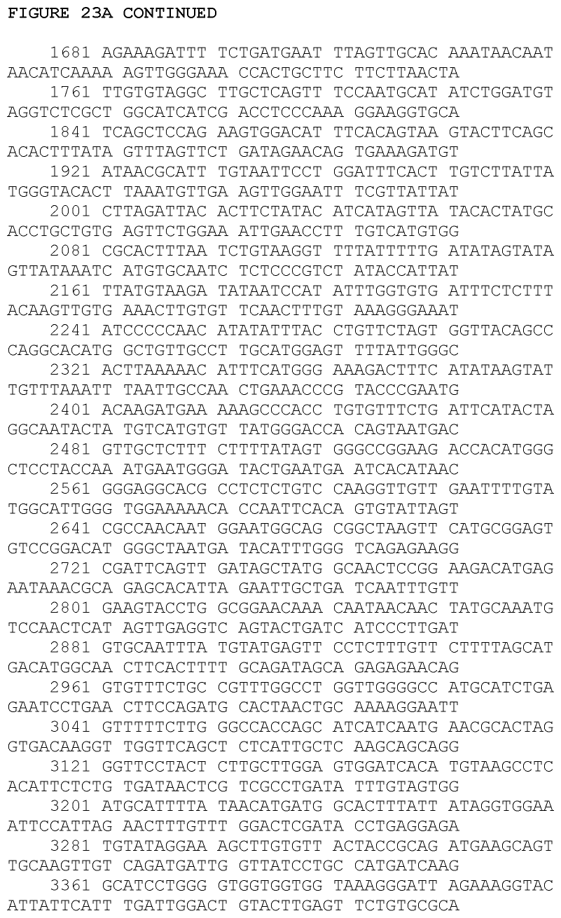

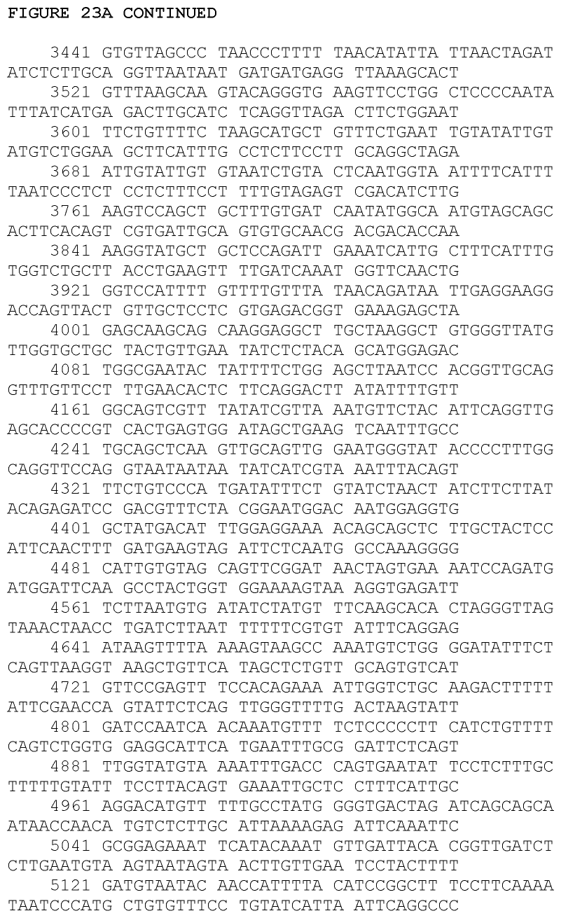

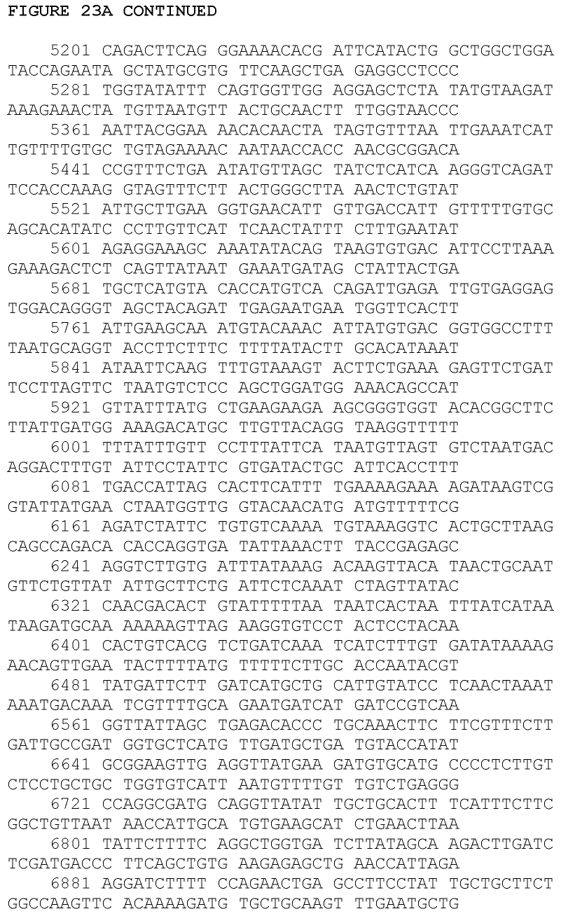

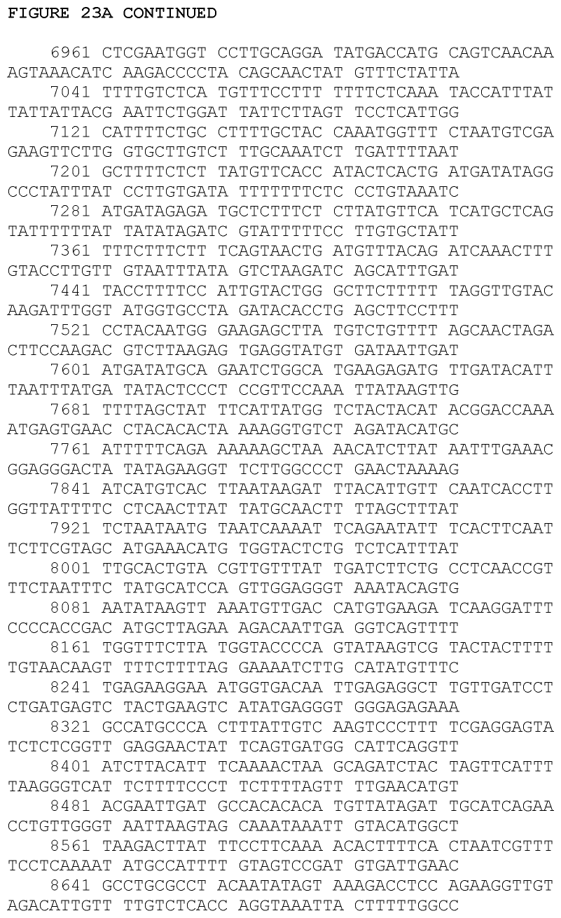

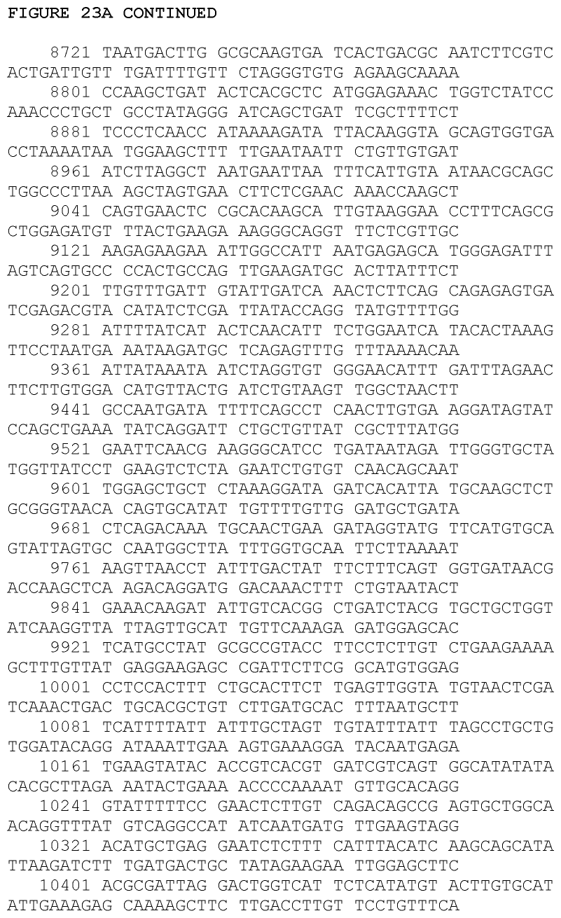

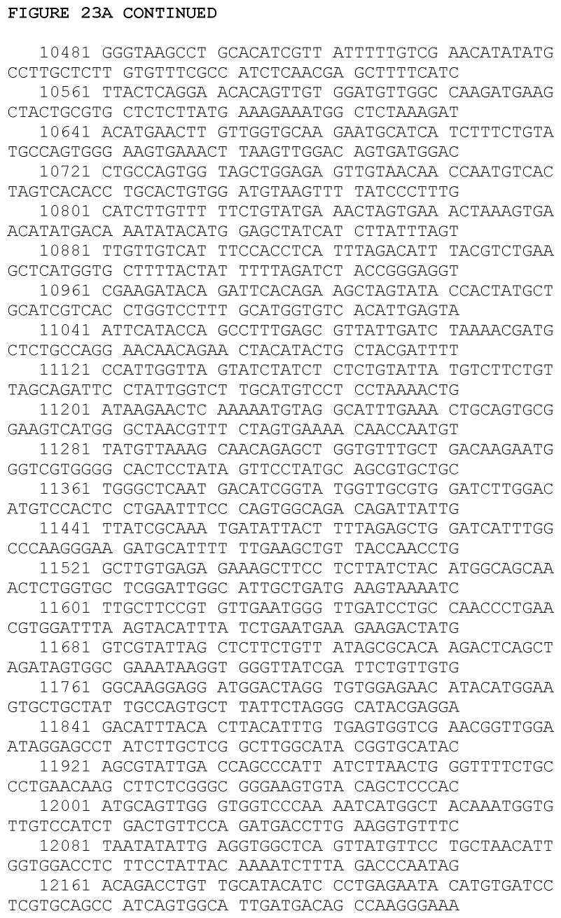

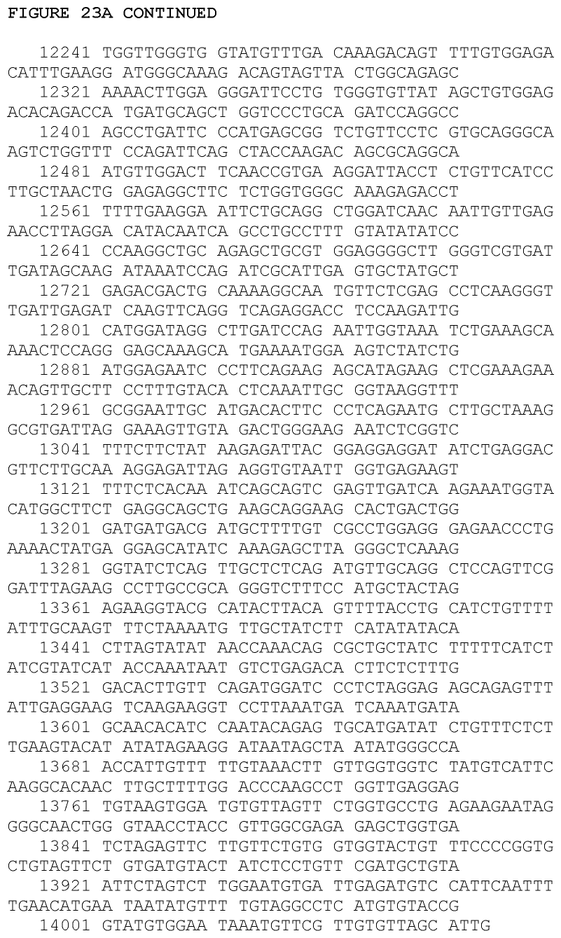

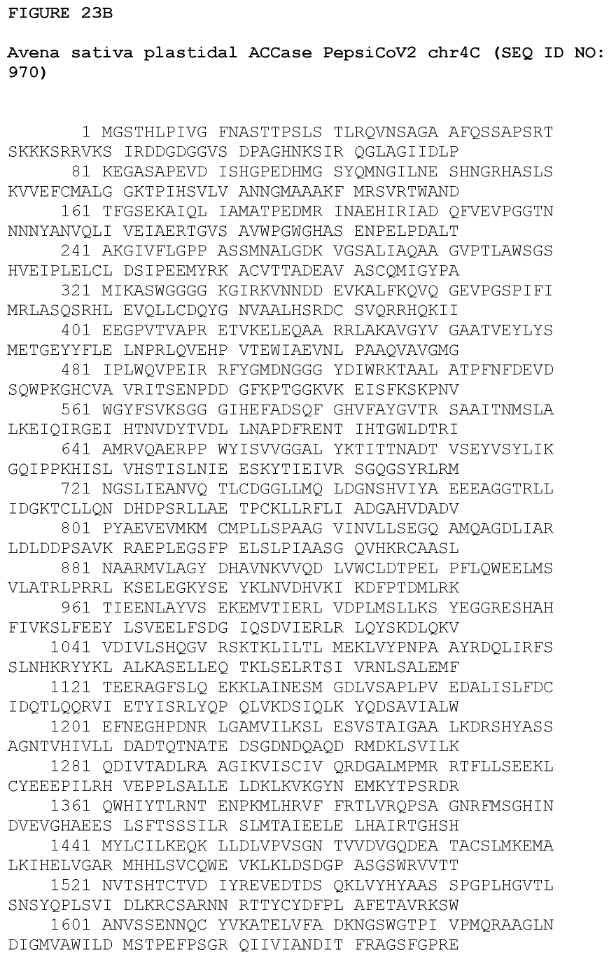

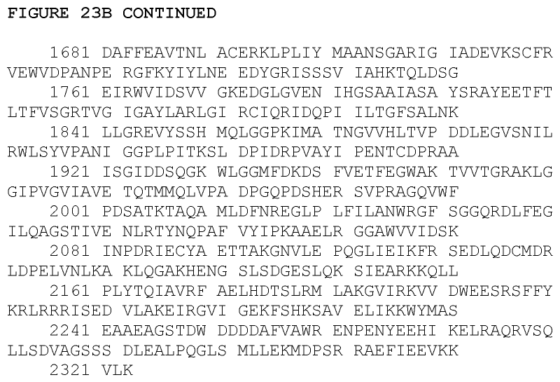

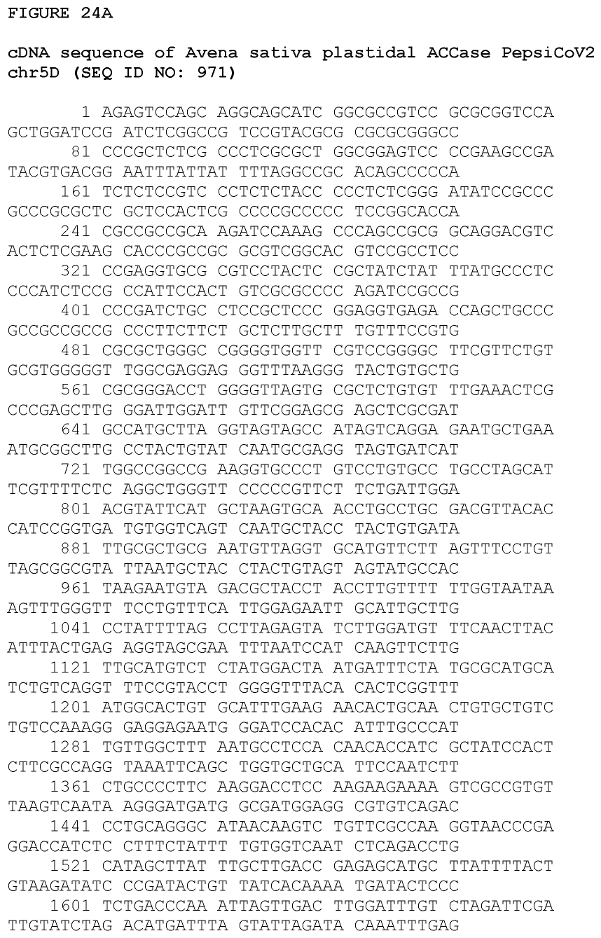

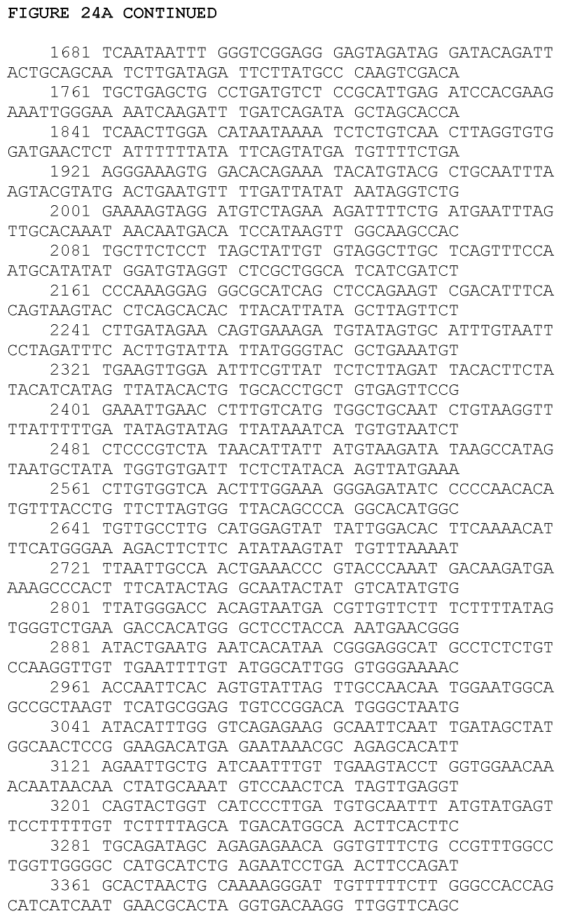

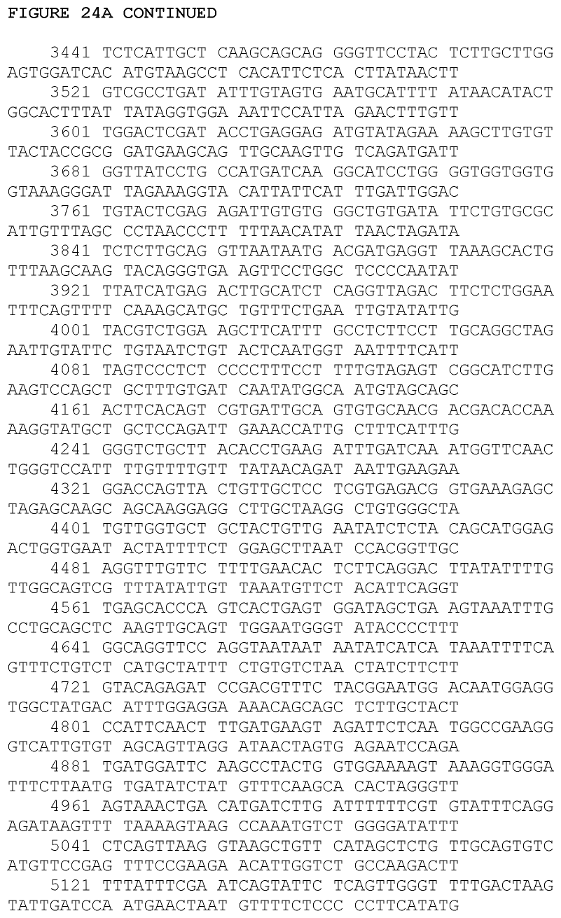

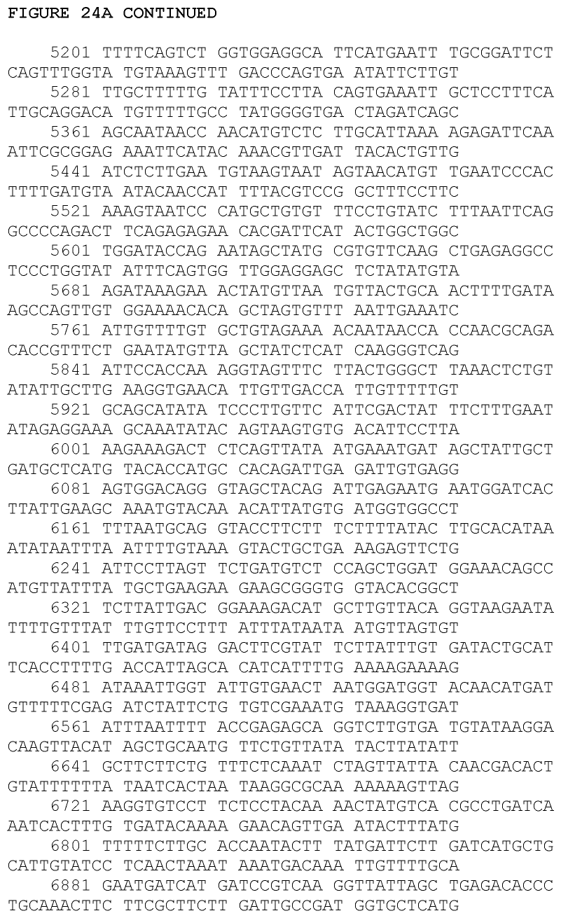

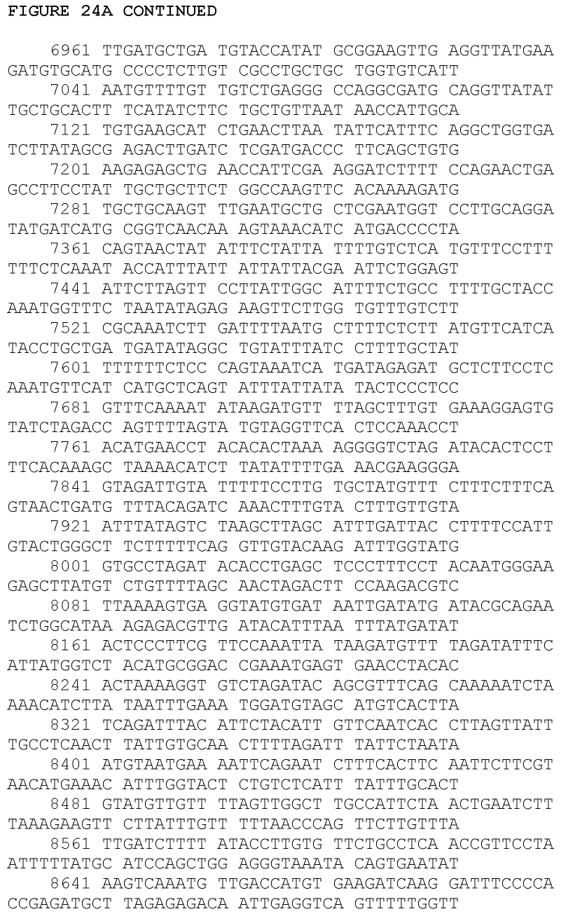

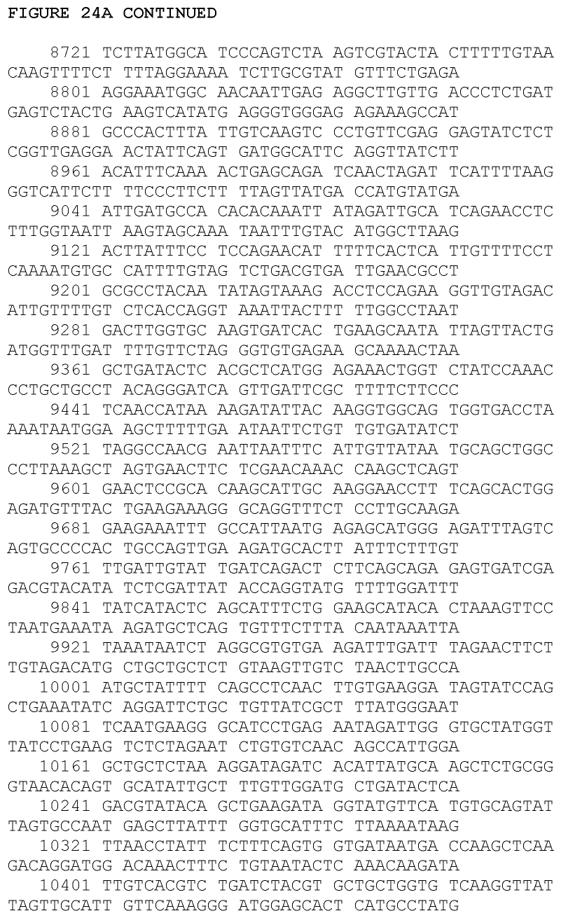

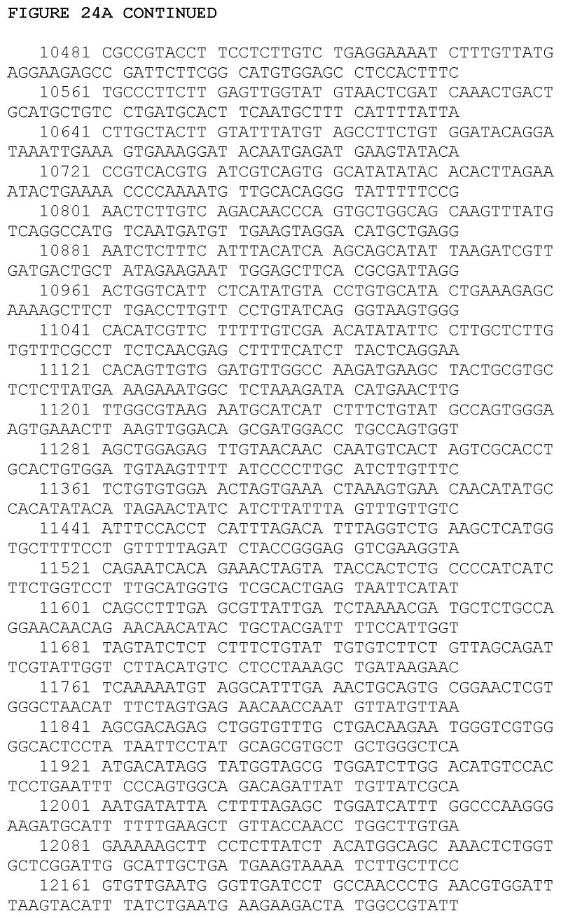

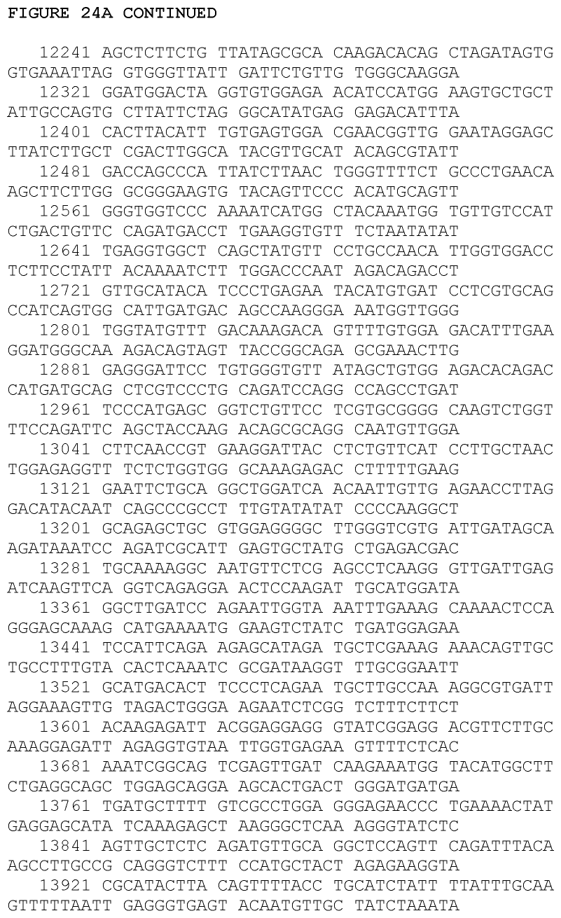

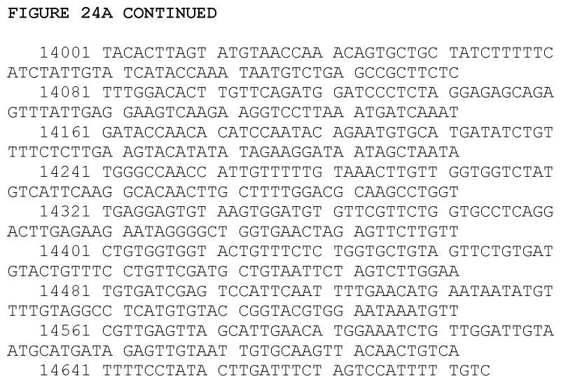

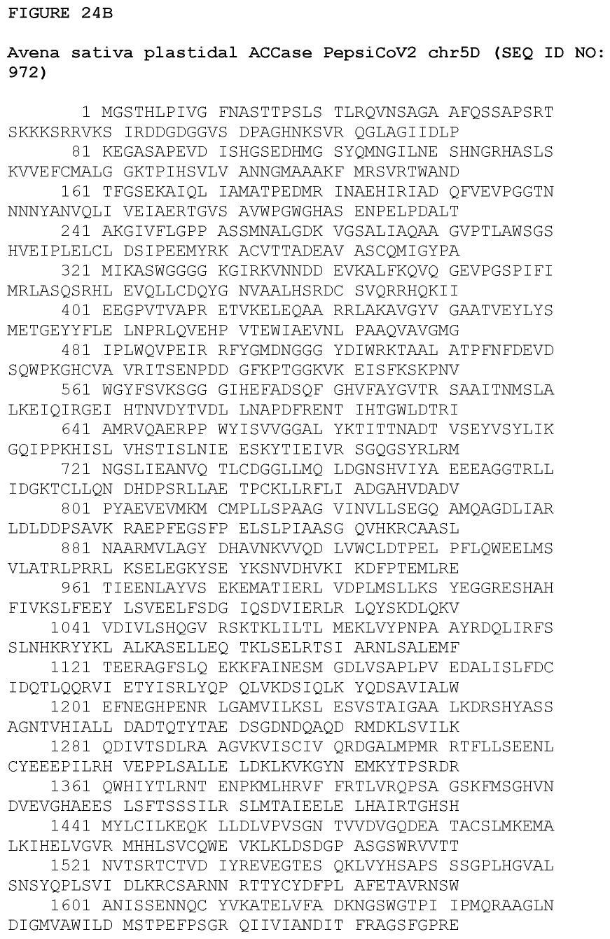

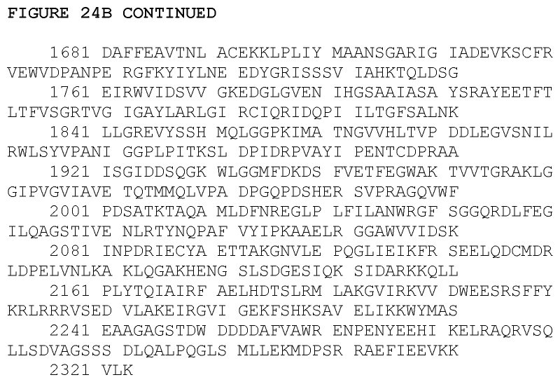

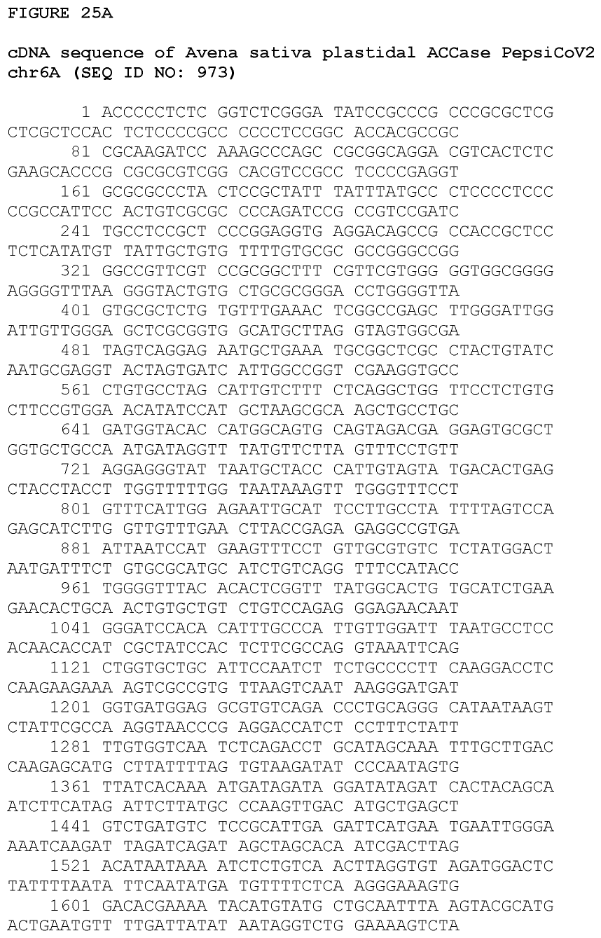

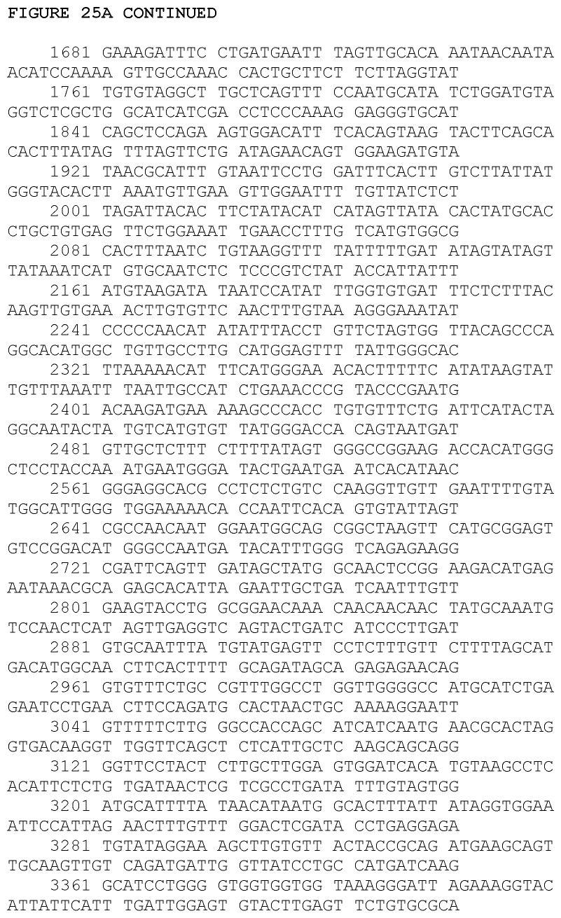

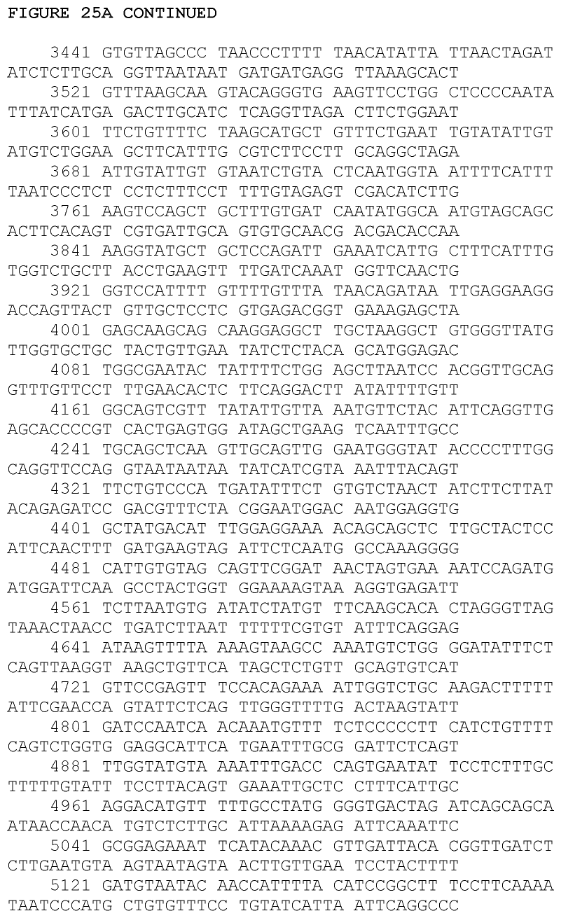

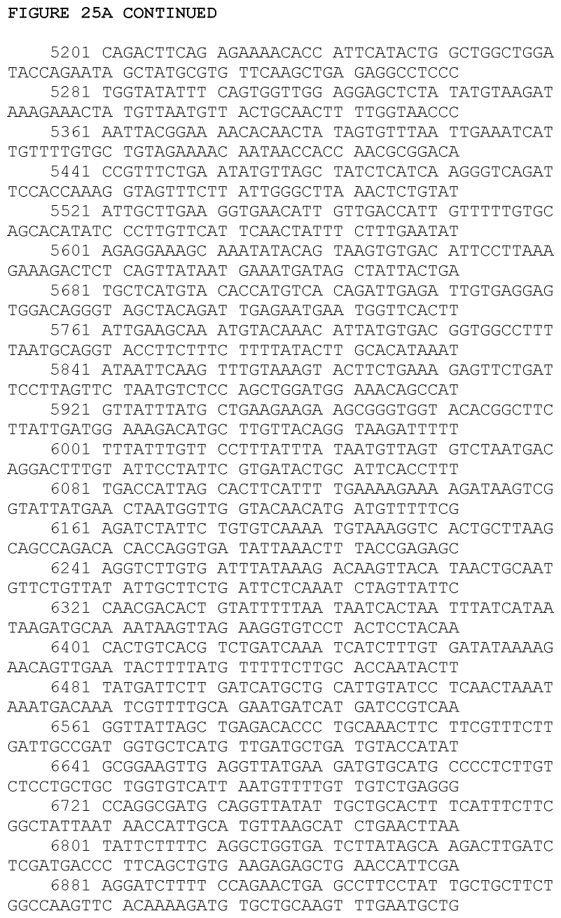

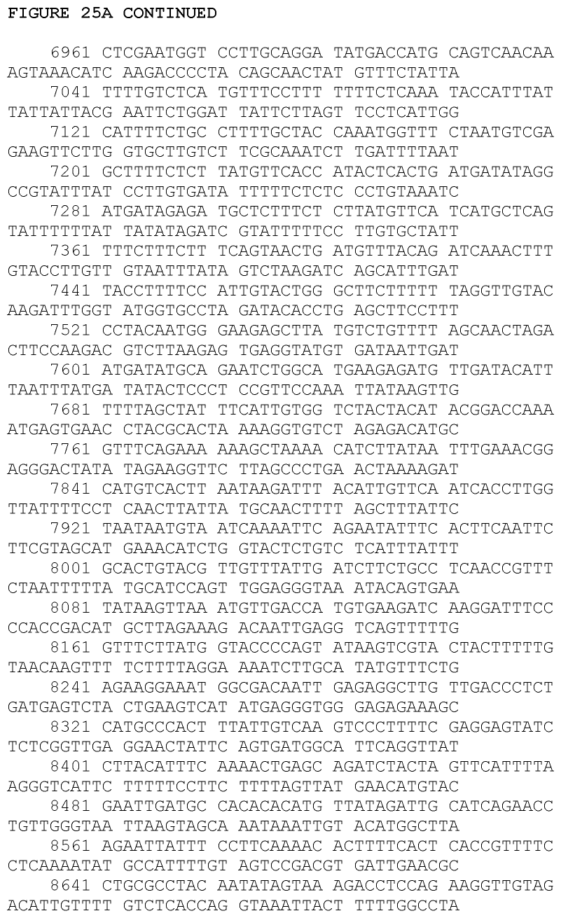

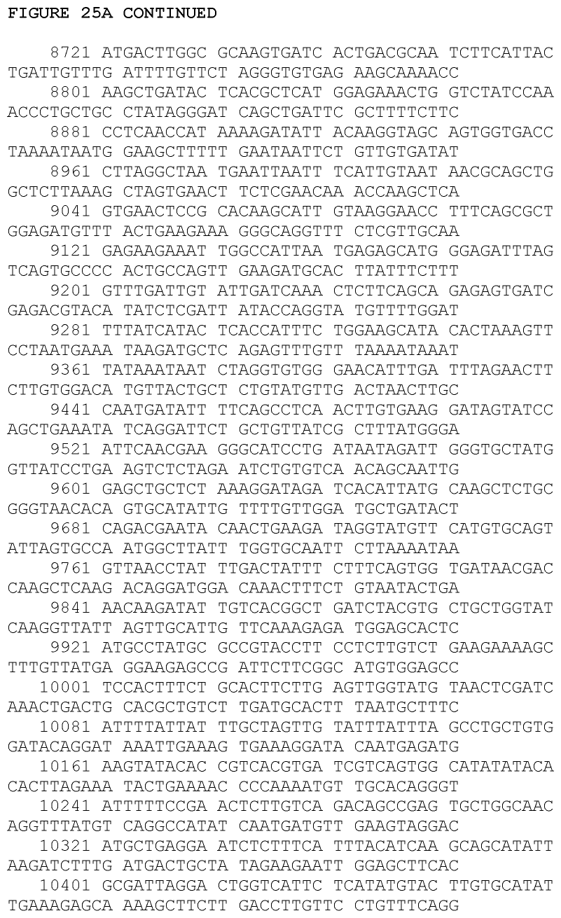

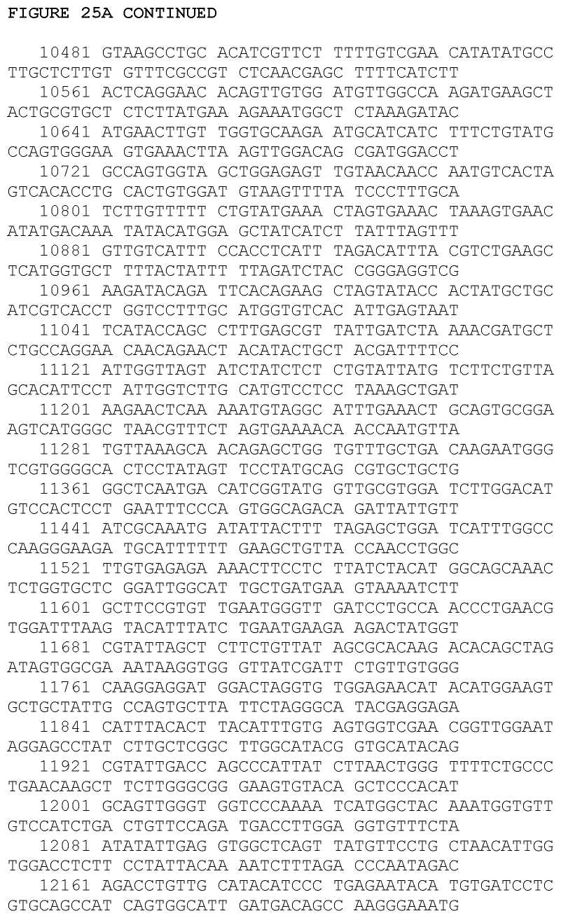

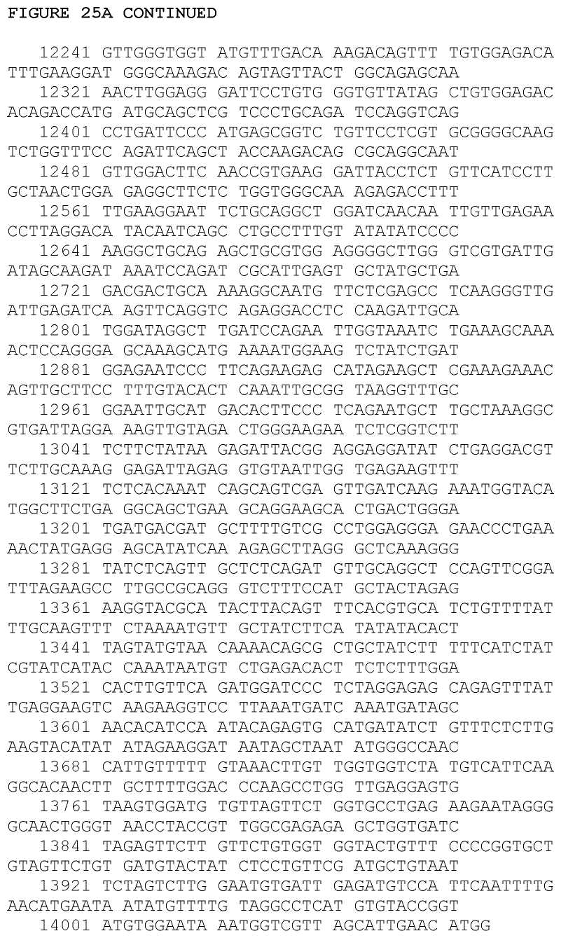

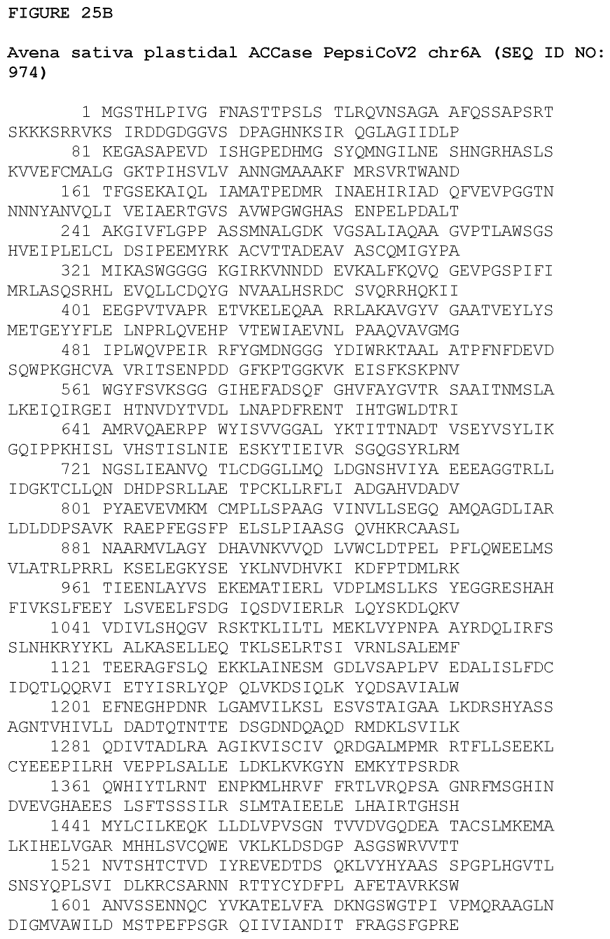

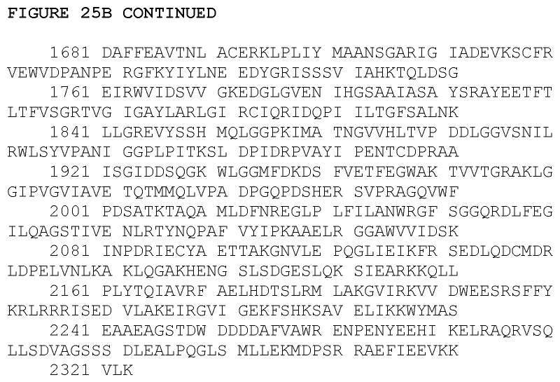

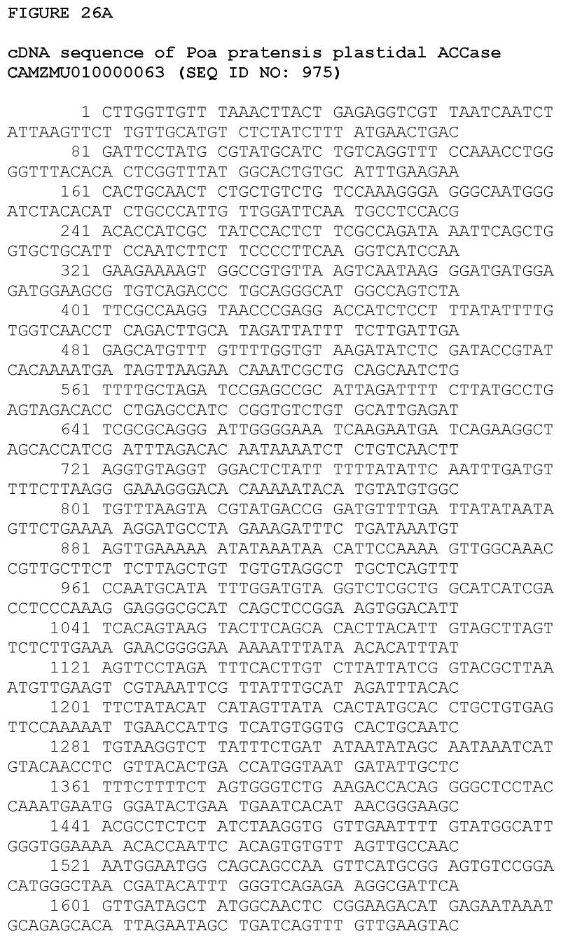

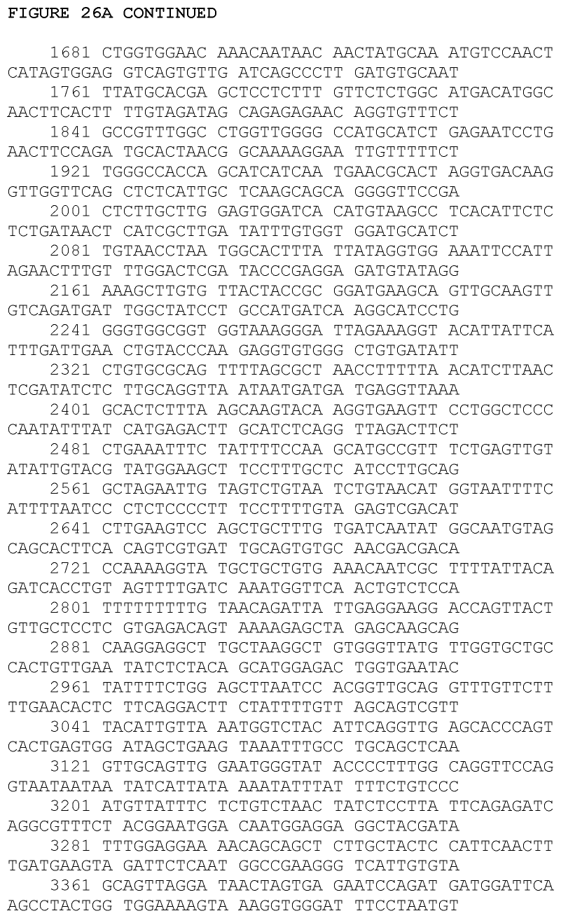

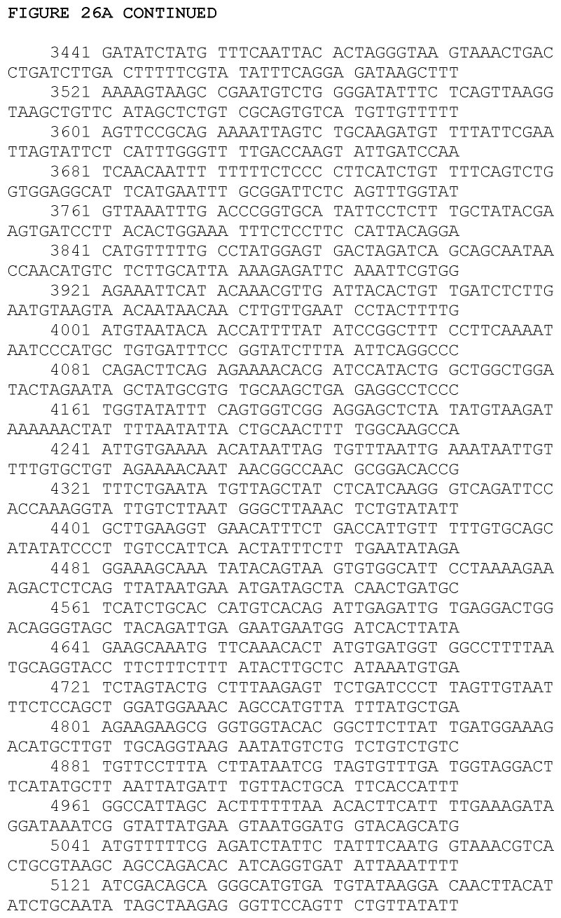

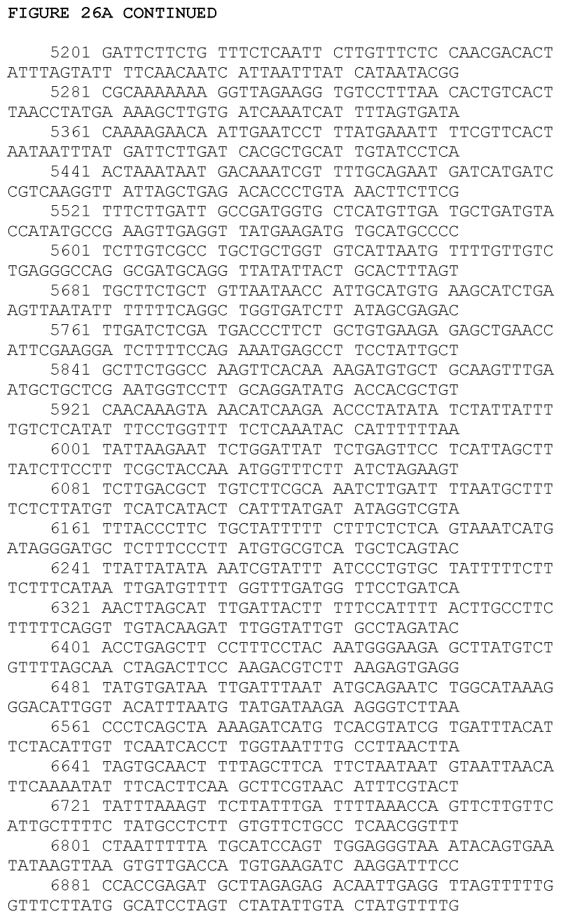

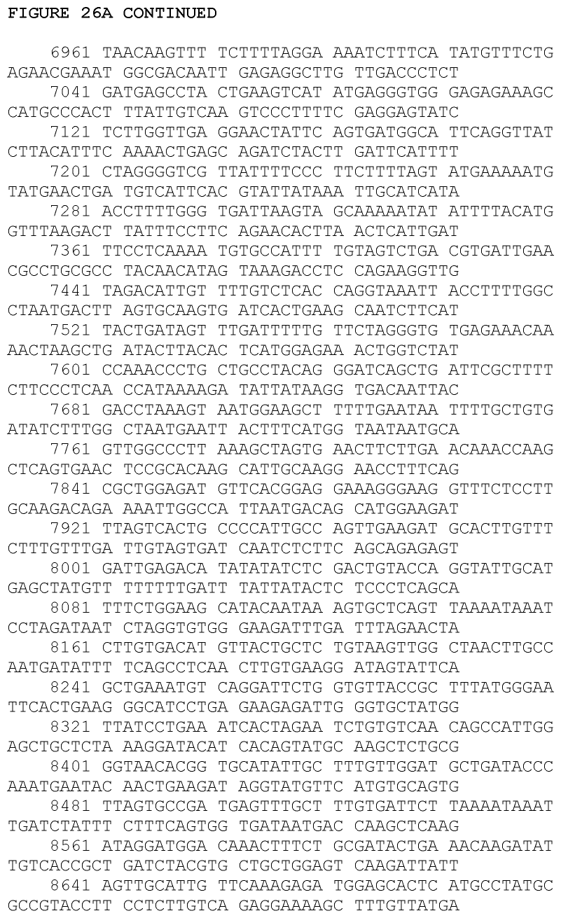

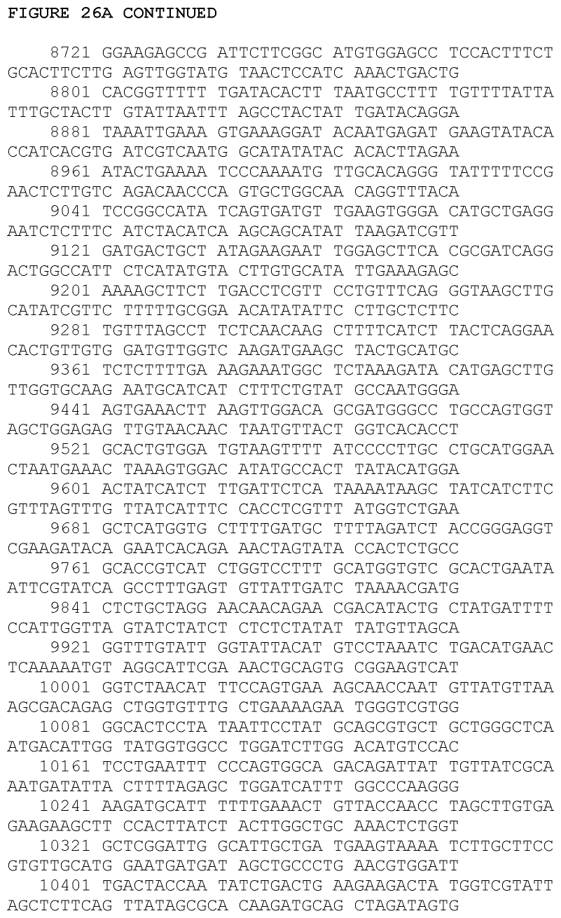

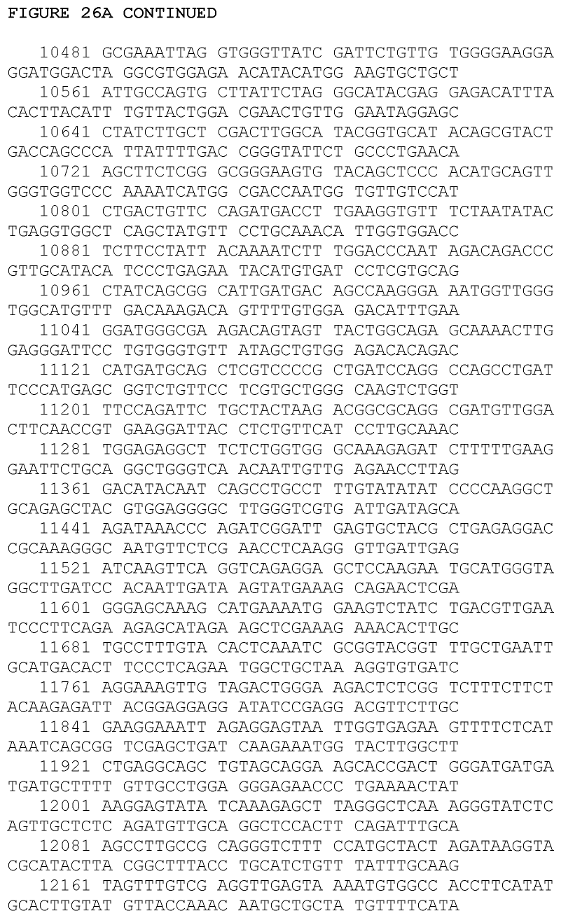

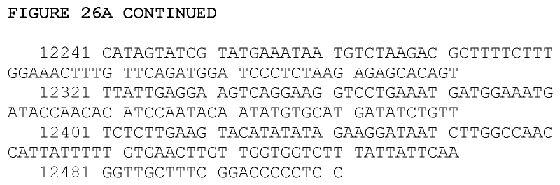

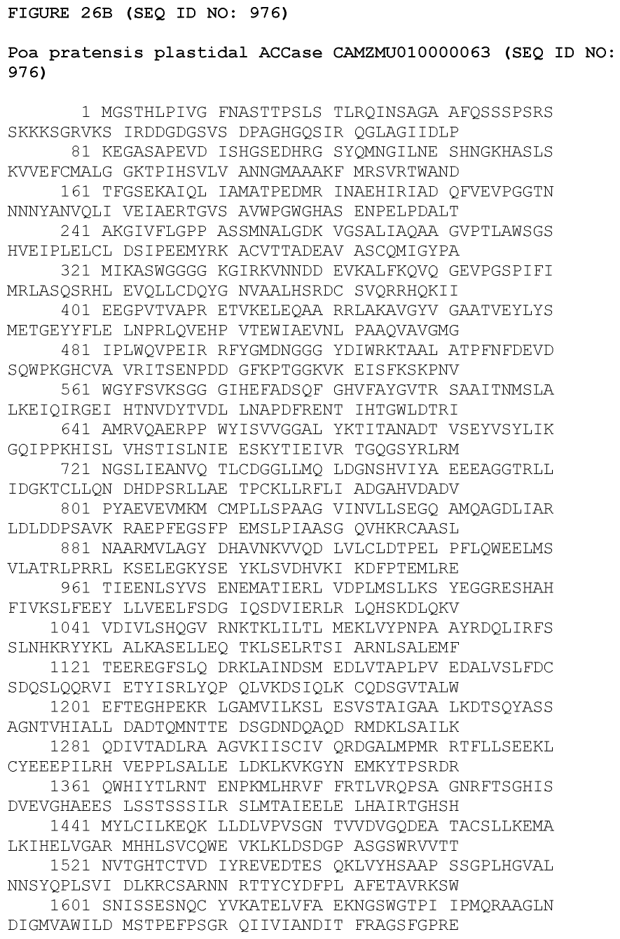

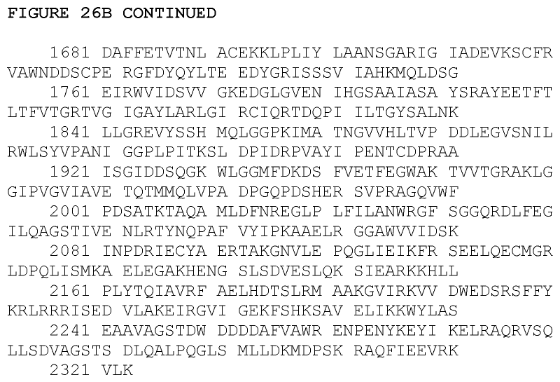

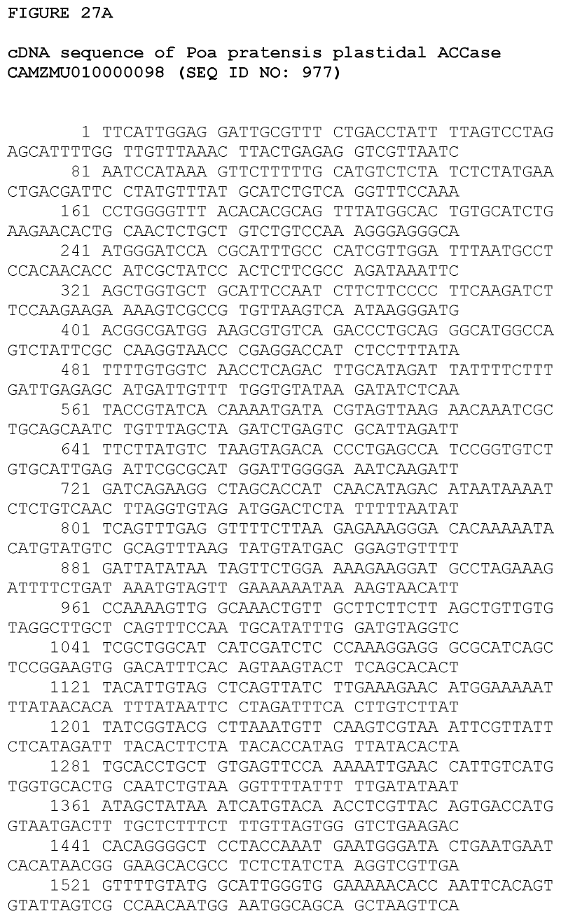

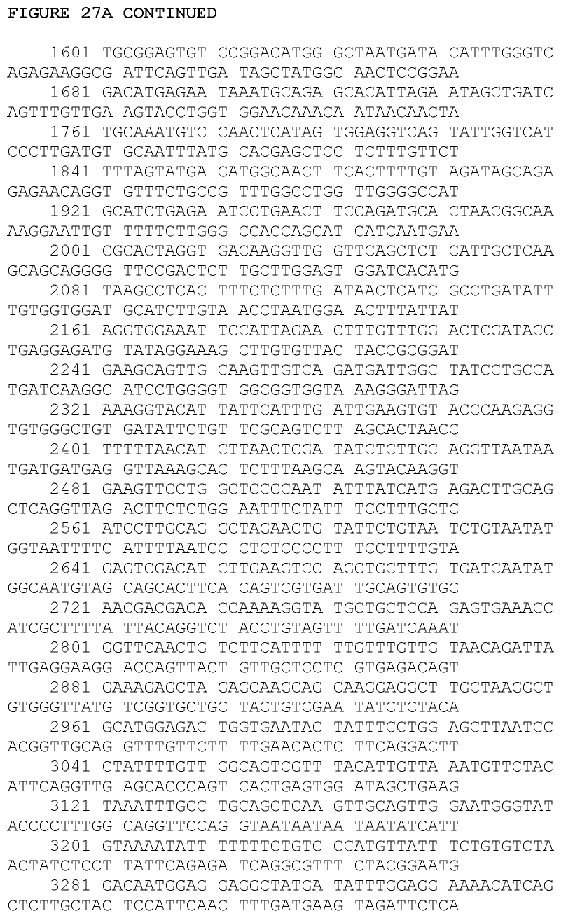

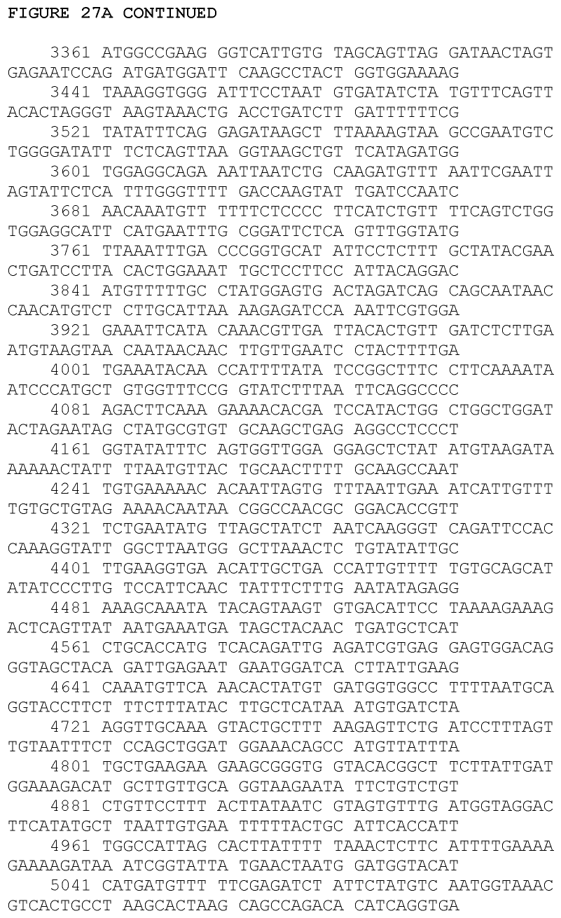

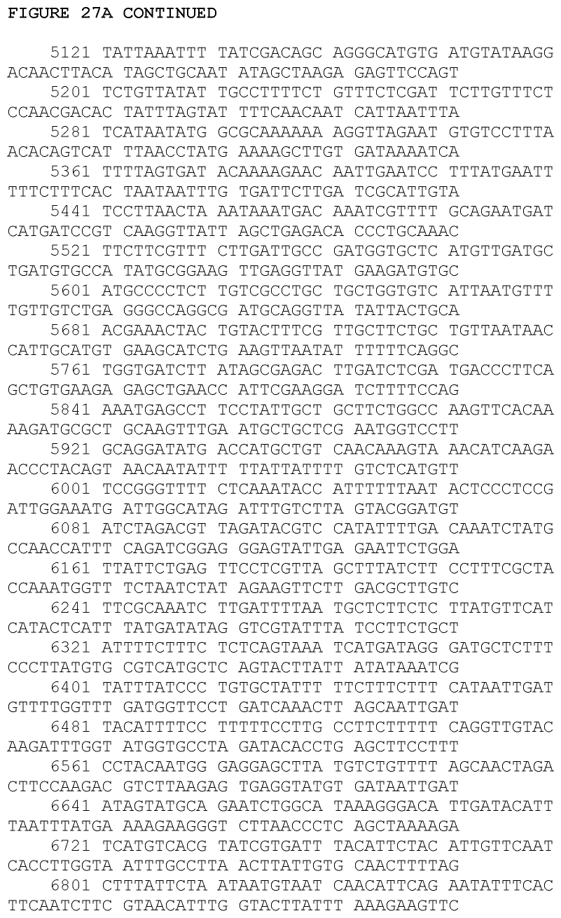

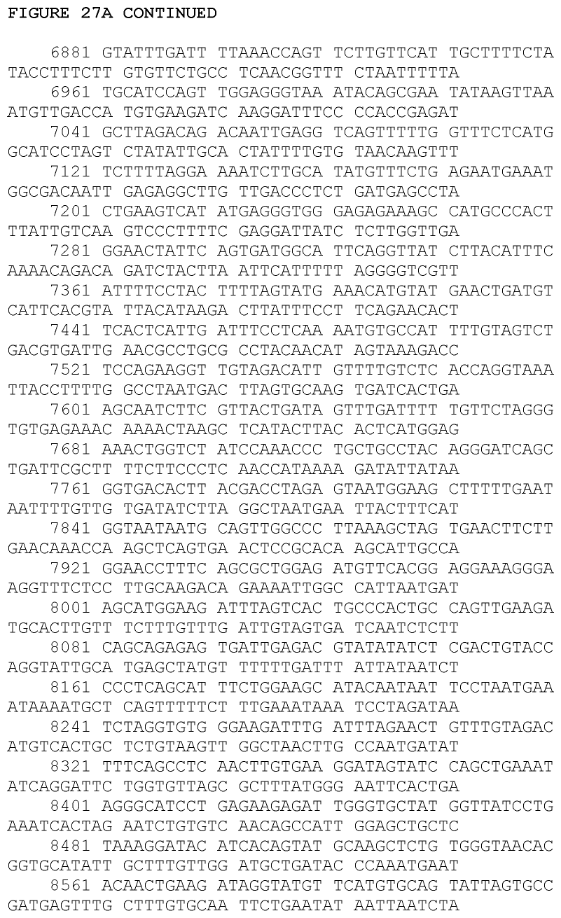

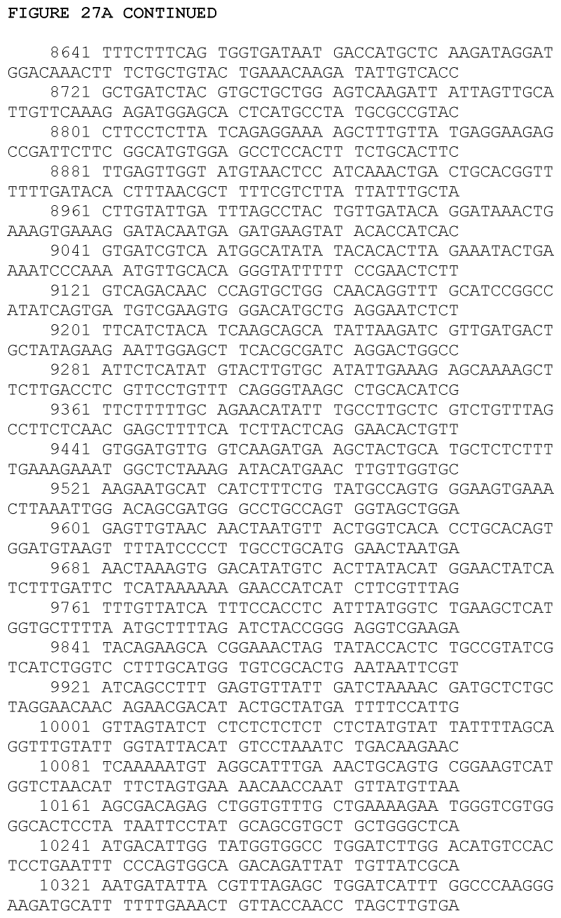

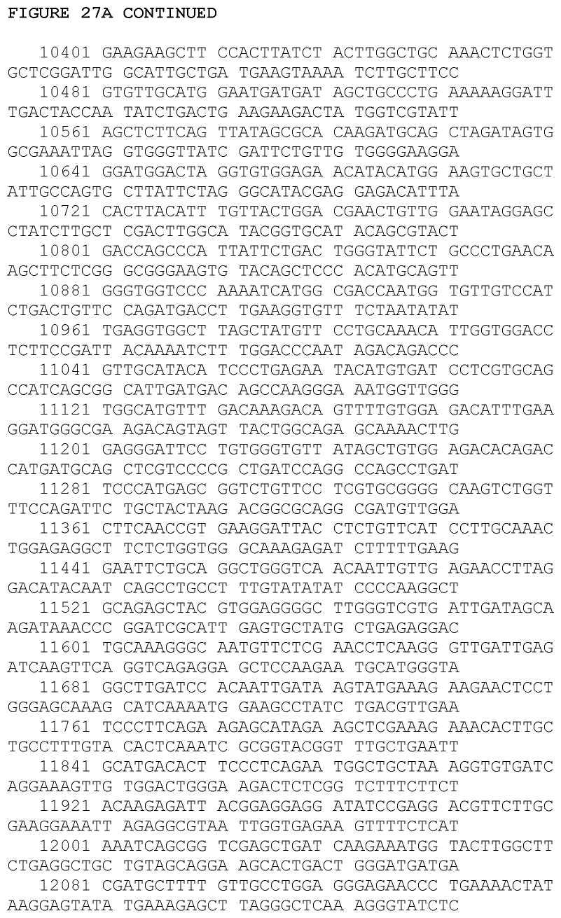

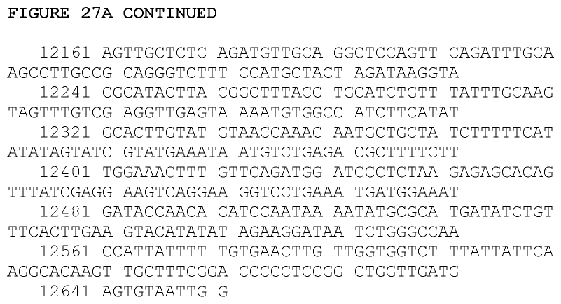

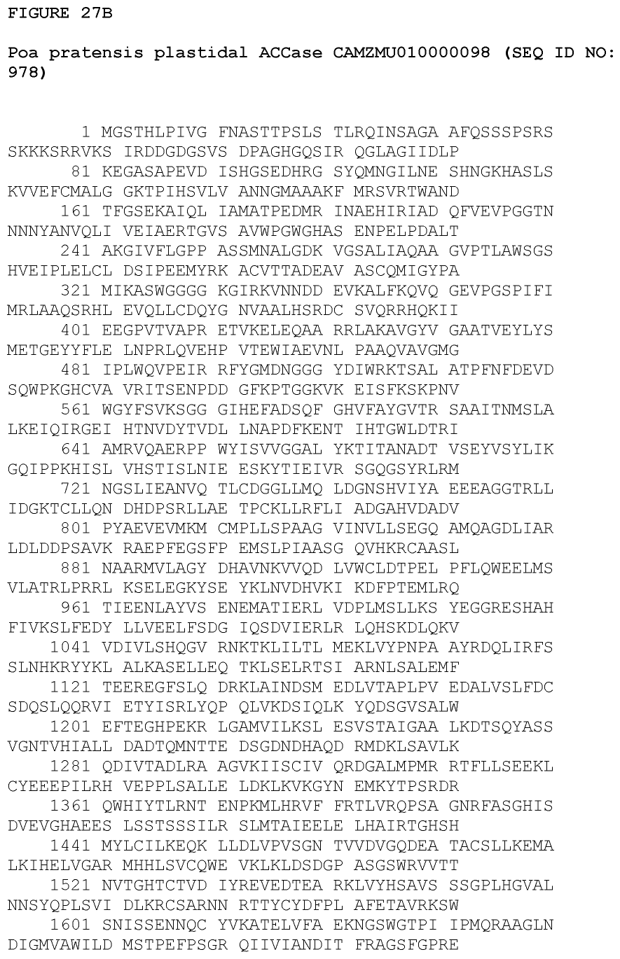

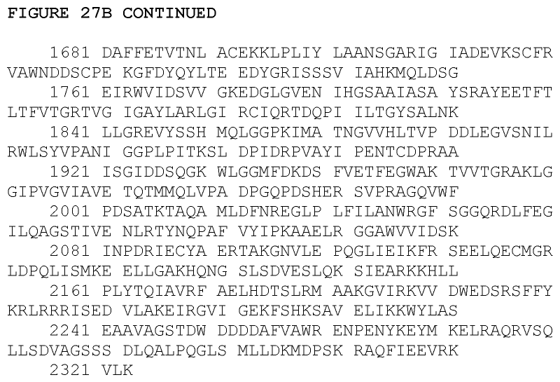

The Oryza sativa ACCase gene product has the following sequence (Swiss-Prot B9FK36, SEQ ID NO: 4; FIG. 2B):

```
                10 20 30 40 50 60
MTSTHVATLG VGAQAPPRHQ KKSAGTAFVS SGSSRPSYRK NGQRTRSLRE ESNGGVSDSK
                70 80 90 100 110 120
KLNHSIRQGL AGIIDLPNDA ASEVDISHGS EDPRGPTVPG SYQMNGIINE THNGRHASVS
              130 140 150 160 170 180
KVVEFCTALG GKTPIHSVLV ANNGMAAAKF MRSVRTWAND TFGSEKAIQL IAMATPEDLR
              190 200 210 220 230 240
INAEHIRIAD QFVEVPGGTN NNNYANVQLI VEIAERTGVS AVWPGWGHAS ENPELPDALT
              250 260 270 280 290 300
AKGIVFLGPP ASSMHALGDK VGSALIAQAA GVPTLAWSGS HVEVPLECCL DSIPDEMYRK
              310 320 330 340 350 360
ACVTTTEEAV ASCQVVGYPA MIKASWGGGG KGIRKVHNDD EVRTLFKQVQ GEVPGSPIFI
              370 380 390 400 410 420
MRLAAQSRHL EVQLLCDQYG NVAALHSRDC SVQRRHQKII EEGPVTVAPR ETVKELEQAA
              430 440 450 460 470 480
RRLAKAVGYV GAATVEYLYS METGEYYFLE LNPRLQVEHP VTEWIAEVNL PAAQVAVGMG
              490 500 510 520 530 540
IPLWQIPEIR RFYGMNHGGG YDLWRKTAAL ATPFNFDEVD SKWPKGHCVA VRITSEDPDD
              550 560 570 580 590 600
GFKPTGGKVK EISFKSKPNV WAYFSVKSGG GIHEFADSQF GHVFAYGTTR SAAITTMALA
              610 620 630 640 650 660
LKEVQIRGEI HSNVDYTVDL LNASDFRENK IHTGWLDTRI AMRVQAERPP WYISVVGGAL
              670 680 690 700 710 720
YKTVTANTAT VSDYVGYLTK GQIPPKHISL VYTTVALNID GKKYTIDTVR SGHGSYRLRM
              730 740 750 760 770 780
NGSTVDANVQ ILCDGGLLMQ LDGNSHVIYA EEEASGTRLL IDGKTCMLQN DHDPSKLLAE
              790 800 810 820 830 840
TPCKLLRFLV ADGAHVDADV PYAEVEVMKM CMPLLSPASG VIHVVMSEGQ AMQAGDLIAR
              850 860 870 880 890 900
LDLDDPSAVK RAEPFEDTFP QMGLPIAASG QVHKLCAASL NACRMILAGY EHDIDKVVPE
              910 920 930 940 950 960
LVYCLDTPEL PFLQWEELMS VLATRLPRNL KSELEGKYEE YKVKFDSGII NDFPANMLRV
              970 980 990 1000 1010 1020
IIEENLACGS EKEKATNERL VEPLMSLLKS YEGGRESHAH FVVKSLFEEY LYVEELFSDG
            1030 1040 1050 1060 1070 1080
IQSDVIERLR LQHSKDLQKV VDIVLSHQSV RNKTKLILKL MESLVYPNPA AYRDQLIRFS
            1090 1100 1110 1120 1130 1140
SINHKAYYKL ALKASELLEQ TKLSELRARI ARSLSELEMF TEESKGLSMH KREIAIKESM
            1150 1160 1170 1180 1190 1200
EDLVTAPLPV EDALISLFDC SDTTVQQRVI ETYIARLYQP HLVKDSIKMK WIESGVIALW
            1210 1220 1230 1240 1250 1260
EFPEGHFDAR NGGAVLGDKR WGAMVIVKSL ESLSMAIRFA LKETSHYTSS EGNMMHIALL
            1270 1280 1290 1300 1310 1320
GADNKMHIIQ ESGDDADRIA KLPLILKDNV TDLHASGVKT ISFIVQRDEA RMTMRRTFLW
            1330 1340 1350 1360 1370 1380
SDEKLSYEEE PILRHVEPPL SALLELDKLK VKGYNEMKYT PSRDRQWHIY TLRNTENPKM
            1390 1400 1410 1420 1430 1440
LHRVFFRTLV RQPSVSNKFS SGQIGDMEVG SAEEPLSFTS TSILRSLMTA IEELELHAIR
            1450 1460 1470 1480 1490 1500
TGHSHMYLHV LKEQKLLDLV PVSGNTVLDV GQDEATAYSL LKEMAMKIHE LVGARMHHLS
            1510 1520 1530 1540 1550 1560
VCQWEVKLKL DCDGPASGTW RIVTTNVTSH TCTVDIYREM EDKESRKLVY HPATPAAGPL
            1570 1580 1590 1600 1610 1620
HGVALNNPYQ PLSVIDLKRC SARNNRTTYC YDFPLAFETA VRKSWSSSTS GASKGVENAQ
            1630 1640 1650 1660 1670 1680
CYVKATELVF ADKHGSWGTP LVQMDRPAGL NDIGMVAWTL KMSTPEFPSG REIIVVANDI
            1690 1700 1710 1720 1730 1740
TFRAGSFGPR EDAFFEAVTN LACEKKLPLI YLAANSGARI GIADEVKSCF RVGWSDDGSP
            1750 1760 1770 1780 1790 1800
ERGFQYIYLS EEDYARIGTS VIAHKMQLDS GEIRWVIDSV VGKEDGLGVE NIHGSAAIAS
            1810 1820 1830 1840 1850 1860
AYSRAYKETF TLTFVTGRTV GIGAYLARLG IRCIQRLDQP IILTGYSALN KLLGREVYSS
            1870 1880 1890 1900 1910 1920
HMQLGGPKIM ATNGVVHLTV SDDLEGVSNI LRWLSYVPAY IGGPLPVTTP LDPPDRPVAY
            1930 1940 1950 1960 1970 1980
IPENSCDPRA AIRGVDDSQG KWLGGMFDKD SFVETFEGWA KTVVTGRAKL GGIPVGVIAV
            1990 2000 2010 2020 2030 2040
ETQTMMQTIP ADPGQLDSRE QSVPRAGQVW FPDSATKTAQ ALLDFNREGL PLFILANWRG
            2050 2060 2070 2080 2090 2100
FSGGQRDLFE GILQAGSTIV ENLRTYNQPA FVYIPMAAEL RGGAWVVVDS KINPDRIECY
            2110 2120 2130 2140 2150 2160
AERTAKGNVL EPQGLIEIKF RSEELQDCMS RLDPTLIDLK AKLEVANKNG SADTKSLQEN
            2170 2180 2190 2200 2210 2220
IEARTKQLMP LYTQIAIRFA ELHDTSLRMA AKGVIKKVVD WEESRSFFYK RLRRRISEDV
            2230 2240 2250 2260 2270 2280
LAKEIRAVAG EQFSHQPAIE LIKKWYSASH AAEWDDDDAF VAWMDNPENY KDYIQYLKAQ
            2290 2300 2310 2320
RVSQSLSSLS DSSSDLQALP QGLSMLLDKM DPSRRAQLVE EIRKVLG
```

Some species have more than one ACCase gene. In such a case one or more of the genes are mutated according to the present description to make herbicide resistant mutant. If the expression levels of the various ACCase genes is known and is different, then it is preferred to mutate the higher expressing ACCase genes. In a preferred embodiment all of the ACCase genes in a crop are mutated to make a one or more of alloxydim, butroxydim, clethodim, cloproxydim, cycloxydim, sethoxydim, tepraloxydim, tralkoxydim, chlorazifop, clodinafop, clofop, diclofop, fenoxaprop, fenoxaprop-P, fenthiaprop, fluazifop, fluazifop-P, haloxyfop, haloxyfop-P, isoxapyrifop, propaquizafop, quizalofop, quizalofop-P, trifop, pinoxaden, agronomically acceptable salts and esters of any of these herbicides, and combinations thereof phenotype.

Mutations and mutant ACCase genes that will produce a gene product that provides resistance to one or more herbicides can be identified, for example, using techniques known in the art. For example, in vitro screening can be done in a microbial system to save time and resources. Growth curves of microbial colonies expressing candidate mutant ACCase genes can be generated to evaluate the mutant ACCase genes in providing a phenotype having resistance to one or more herbicides. For example, U.S. Pat. No. 6,306,636 discloses a Saccharomyces cerevisiae ACC1 mutant complemented with a chimeric wheat cytosolic ACCase construct to identify one or more of alloxydim, butroxydim, clethodim, cloproxydim, cycloxydim, sethoxydim, tepraloxydim, tralkoxydim, chlorazifop, clodinafop, clofop, diclofop, fenoxaprop, fenoxaprop-P, fenthiaprop, fluazifop, fluazifop-P, haloxyfop, haloxyfop-P, isoxapyrifop, propaquizafop, quizalofop, quizalofop-P, trifop, pinoxaden, agronomically acceptable salts and esters of any of these herbicides, and combinations thereof resistance assay employing Oryza mutant ACCase genes transformed into a Saccharomyces cerevisiae strain lacking the native acc1 gene. Growth curve assays and enzymatic assays measuring Kᵢ and Kₘ values for candidate mutants are conducted according to well-known assay techniques.

In various aspects and embodiments of the present disclosure, provided include a plant having one or more ACCase mutations and/or mutation combinations selected from the group consisting of the mutations/combinations listed on Table 1 below and/or methods of making such a plant. As denoted at the bottom of Table 1, some amino acids in certain ACCase proteins (e.g., the rice plastidal ACCase protein) are not found in the blackgrass (Alopecurus myosuroides) protein sequence. These positions have been noted with a double number identification that indicates the surrounding positions in the blackgrass (Alopecurus myosuroides) protein. By way of example “rice G1605-6A*” refers to a glycine residue in the rice ACCase protein that lies between residues 1605 and 1606 of the blackgrass (Alopecurus myosuroides) ACCase sequence and has been substituted with an alanine residue.

TABLE 1
Exemplary mutations; position numbering is rcited relative
to blackgrass (Alopecurus myosuroides) plastidal
ACCase positions (FIG. 1B; SEQ ID NO. 2). By way of
example, an N-to-S substitution at postion 1460 of SEQ
ID NO: 2 may be written as “N → S” or “N1460S.”

| ACCase Position | Specific Changes and/or Specific Combinations |
|---|---|
| 1460 | N → S |
| 1529 | V → M |
| 1529 | V1529M + L1818H |
| 1529 | V1529M + L1818I |
| 1543 | L → F |
| 1543 | L → V |
| 1543 | L1543F + D1681E |
| 1543 | L1543F + D1681G |
| 1543 | L1543V + D1681E |
| 1543 | L1543V + D1681G |
| 1555 | L → P |
| 1555 | L1555P + S1605Y + D2078G |
| 1575 | C → S |
| 1575 | C1575S + G1605-6A* + V2075L |
| 1605 | S → Y |
| 1605 | S1605Y + V2098A |
| 1605 | S1605Y + L1555P + D2078G |
| 1605-6* | G → A |
| 1605-6* | G1605-6A* + C1575S + V2075L |
| 1607 | N → S |
| 1649 | L → M |
| 1649 | L1649M + V1715A + D2078G |
| 1672 | R → I |
| 1672 | R1672I + D2078G |
| 1672 | R1672I + M1975T + D2078G |
| 1681 | D → E |
| 1681 | D → G |
| 1681 | D1681E + L1543V |
| 1681 | D1681E + L1543F |
| 1681 | D1681G + L1543V |
| 1681 | D1681G + L1543F |
| 1683 | F → L |
| 1683 | F1683L + A1752T + K1930N + V2075L + E2169D |
| 1715 | V → A |
| 1715 | V1715A + L1649M + D2078G |
| 1725 | D → E |
| 1725 | D1725E + I1781T + V2075L |
| 1734 | R → G |
| 1738 | M → L |
| 1739 | T → S |
| 1752 | A → T |
| 1752 | A1752T + F1683L + K1930N + V2075L + E2169D |
| 1756 | Q → P |
| 1756 | Q → E |
| 1766 | I → V |
| 1766 | I1766V + D2078G + D2127N |
| 1772 | K → E |
| 1772 | K → N |
| 1772 | K1772E + S1784G |
| 1772 | K1772E + S1784N |
| 1772 | K1772E + S1784G |
| 1772 | K1772E + S1784N |
| 1780 | N → D |
| 1780 | N1780D + I1781L |
| 1781 | I → L |
| 1781 | I → T |
| 1781 | I → V |
| 1781 | I → A |
| 1781 | I1781L + N1780D |
| 1781 | I1781L + A1785G |
| 1781 | I1781L + Q1824P |
| 1781 | I1781L + V1864F |
| 1781 | I1781L + W1999G |
| 1781 | I1781L + W2027C |
| 1781 | I1781L + W2027R |
| 1781 | I1781L + I2041N |
| 1781 | I1781L + V2049F |
| 1781 | I1781L + V2075L |
| 1781 | I1781L + D2078G |
| 1781 | I1781L + C2088M |
| 1781 | I1781L + C2088R |
| 1781 | I 1781L + V2098G |
| 1781 | I1781L + A1785G + C2088M |
| 1781 | I1781T + V2075L |
| 1781 | I1781T + D1725E + V2075L |
| 1783 | G → C |
| 1784 | S → G |
| 1784 | S → N |
| 1784 | S1784G + L1818H |
| 1784 | S1784G + L1818I |
| 1784 | S1784N + L1818H |
| 1784 | S1784N + L1818I |
| 1784 | S1784G + K1772E |
| 1784 | S1784N + K1772E |
| 1785 | A → G |
| 1785 | A1785G + I1781L |
| 1785 | A1785G + D2078G |
| 1785 | A1785G + V2098A |
| 1785 | A1785G + V2098G |
| 1785 | I1781L + A1785G + C2088M |
| 1786 | A → P |
| 1792 | S → L |
| 1792 | S1792L + V2049F |
| 1811 | I → N |
| 1818 | L → H |
| 1818 | L → I |
| 1818 | L1818H + V1534M |
| 1818 | L1818H + K1840M |
| 1818 | L1818H + K1840R |
| 1818 | L1818H + S1784N |
| 1818 | L1818H + S1784G |
| 1818 | L1818I + S1784N |
| 1818 | L1818I + S1784G |
| 1818 | L1818I + V1534M |
| 1818 | L1818I + K1840M |
| 1818 | L1818I + K1840R |
| 1824 | Q → P |
| 1824 | Q1824P + I1781L |
| 1824 | Q1824P + D2078G |
| 1837 | A → V |
| 1837 | A1837V + V2075L |
| 1837 | A1837V + V2075I |
| 1840 | K → R |
| 1840 | K → M |
| 1840 | K1840R + L1818H |
| 1840 | K1840M + L1818H |
| 1840 | K1840R + L1818I |
| 1840 | K1840M + L1818I |
| 1864 | V → F |
| 1864 | V1864F + I1781L |
| 1864 | V1864F + W1999G |
| 1870 | P → R |
| 1922 | S → G |
| 1930 | K → N |
| 1930 | K1930N + A1752T + F1683L + V2075L + E2169D |
| 1938 | K → R |
| 1946 | E → G |
| 1946 | E → D |
| 1946 | E → V |
| 1946 | E1946V + D2078G + A2179T + F2253I |
| 1957 | A → T |
| 1957 | A1957T + V2075L |
| 1966 | V → L |
| 1966 | V1966L + D2078G + V2182A + K2317T |
| 1967 | I → V |
| 1967 | I1967V + V2075L |
| 1974 | M → T |
| 1974 | M1974T + D2078G |
| 1975 | M → T |
| 1975 | M1975T + D2078G |
| 1975 | M1975T + R1672I + D2078G |
| 1992 | V → D |
| 1992 | V1992D + V2075L + F2196Y + L2299I |
| 1999 | W → C |
| 1999 | W → G |
| 1999 | W → L |
| 1999 | W → S |
| 1999 | W1999C + V2075I |
| 1999 | W1999G + I1781L |
| 1999 | W1999G + V1864F |
| 1999 | W1999G + V2049I |
| 1999 | W1999G + V2075L |
| 1999 | W1999G + D2078G |
| 1999 | W1999G + G2096A |
| 1999 | W1999G + V2098A |
| 1999 | W1999L + V2049F |
| 2004 | A → V |
| 2112 | E → V |
| 2112 | E2112V + D2078G |
| 2027 | W → C |
| 2027 | W → R |
| 2027 | W2027R + I1781L |
| 2027 | W20207R + I1781L |
| 2039 | E → D |
| 2039 | E → G |
| 2039 | E2039G + D2078G |
| 2041 | I → D |
| 2041 | I → N |
| 2041 | I → V |
| 2041 | I2041N + I1781L |
| 2041 | I2041N + D2078G |
| 2041 | I2041V + D2078G |
| 2041 | I2041N + C2088R |
| 2049 | V → F |
| 2049 | V → I |
| 2049 | V → L |
| 2049 | V → T |
| 2049 | V → C |
| 2049 | V → A |
| 2049 | V → S |
| 2049 | V2049F + I1781L |
| 2049 | V2049F + I1781V |
| 2049 | V2049F + S1792L |
| 2049 | V2049I + W1999G |
| 2049 | V2049F + W1999L |
| 2049 | V2049F + C2088F |
| 2049 | V2049A + D2078G or V2098A |
| 2049 | V2049C + D2078G or V2098A |
| 2049 | V2049L + D2078G or V2098A |
| 2049 | V2049S + D2078G or V2098A |
| 2049 | V2049T + D2078G or V2098A |
| 2049 | V2049I + V2075L + A2178I |
| 2059 | A → V |
| 2059 | A2059G + D2078G |
| 2059 | A2059V + D2078G + S2280P + S2297C |
| 2067 | A → S |
| 2067 | A2067S + V2075L + L2159P |
| 2074 | W → L |
| 2075 | V → M |
| 2075 | V → L |
| 2075 | V → I |
| 2075 | V → G |
| 2075 | V2075L + C1575S + G1605-6A* |
| 2075 | V2075L + I781L |
| 2075 | V2075L + I1781T |
| 2075 | V2075I + A1837V |
| 2075 | V2075I + W1999C |
| 2075 | V2075L + W1999G |
| 2075 | V2075M + K2080E |
| 2075 | V2075L + C2088W |
| 2075 | V2075L + N2097S |
| 2075 | V2075L + I1967V |
| 2075 | V2075L + D1725E + I1781T |
| 2075 | V2075L + K1930N + A1752T + F1683L + E2169D |
| 2078 | D → G |
| 2078 | D → K |
| 2078 | D → T |
| 2078 | D → V |
| 2078 | D2078G + I1249K |
| 2078 | D2078G + G1605-6A* |
| 2078 | D2078G + I1781L |
| 2078 | D2078G + I1785G |
| 2078 | D2078G + Q1824P |
| 2078 | D2078G + W1999G |
| 2078 | D2078G + E2039G |
| 2078 | D2078G + I2041N |
| 2078 | D2078G + I2041V |
| 2078 | D2078G + V2049A/C/L/S/T |
| 2078 | D2078G + A2059G |
| 2078 | D2078G + S2079F |
| 2078 | D2078G + S2079P |
| 2078 | D2078G + P2083T |
| 2078 | D2078G + C2088G |
| 2078 | D2078G + C2088N |
| 2078 | D2078G + N2097S |
| 2078 | D2078G + E2112V |
| 2078 | D2078G + N2262K |
| 2078 | D2078G + I2267V + E2150K |
| 2078 | D2078G + S2276P + K2302Q |
| 2078 | D2078G + L1555P + S1605Y |
| 2078 | D2078G + L1649M + V1715A |
| 2078 | D2078G + R1672I |
| 2078 | D2078G + R1672I + M1975T |
| 2078 | D2078G + I1766V + D2127N |
| 2078 | D2078G + E1946V + A2179T + F2253I |
| 2078 | D2078G + M1974T |
| 2078 | D2078G + G1605-6A* |
| 2078 | D2078G + V1966L + V2182A + K2317T |
| 2078 | D2078G + A2059V + S2280P + S2297C |
| 2078 | D2078G + R2085S + Q2155L |
| 2078 | D2078G + N2097S + H2223R + S2276F |
| 2078 | D2078G + K2154R + K2232N |
| 2078 | D2078G + I2149K |
| 2079 | S → F |
| 2079 | S → P |
| 2079 | S2079F + D2078G |
| 2079 | S2079P + D2078G |
| 2080 | K → E |
| 2080 | K → deleted |
| 2080 | K2080E + V2075M |
| 2081 | I → deleted |
| 2081 | 2080 deleted + 2081 deleted |
| 2083 | P → N |
| 2083 | P → Q |
| 2083 | P → S |
| 2083 | P → T |
| 2083 | P2083T + D2078G |
| 2085 | R → S |
| 2085 | R2085S + D2078G + Q2155L |
| 2088 | C → R |
| 2088 | C → W |
| 2088 | C → F |
| 2088 | C → G |
| 2088 | C → H |
| 2088 | C → K |
| 2088 | C → S |
| 2088 | C → T |
| 2088 | C → L |
| 2088 | C → V |
| 2088 | C → N |
| 2088 | C → M |
| 2088 | C2088R + I1781L |
| 2088 | C2088R + I2041N |
| 2088 | C2088F + V2049F |
| 2088 | C2088W + V2075L |
| 2088 | C2088G + D2078G |
| 2088 | C2088R + D2078G |
| 2088 | C2088W + V2098C |
| 2088 | C2088T + V2098A |
| 2088 | C2088G + V2098G |
| 2088 | C2088H + V2098A |
| 2088 | C2088H + V2098G |
| 2088 | C2088K + V2098A |
| 2088 | C2088L + V2098A |
| 2088 | C2088L + V2098G |
| 2088 | C2088S + V2098G |
| 2088 | C2088V + V2098G |
| 2088 | C2088M + I1781L |
| 2088 | C2088M + I1781L + A1785G |
| 2088 | C2088N + D2078G |
| 2095 | K → E |
| 2095 | K → N |
| 2095 | K2095E + V2075M |
| 2096 | G → A |
| 2096 | G → S |
| 2096 | G2096A + V2098A |
| 2096 | G2096A + W1995G |
| 2097 | N → R |
| 2097 | N → K |
| 2097 | N → E |
| 2097 | N → H |
| 2097 | N → D |
| 2097 | N → A |
| 2097 | N → G |
| 2097 | N → C |
| 2097 | N → V |
| 2097 | N → P |
| 2097 | N → L |
| 2097 | N → I |
| 2097 | N → M |
| 2097 | N → F |
| 2097 | N → W |
| 2097 | N → Q |
| 2097 | N → S |
| 2097 | N → T |
| 2097 | N → Y |
| 2097 | N2097R + V2075L or V2075I |
| 2097 | N2097K + V2075L or V2075I |
| 2097 | N2097E + V2075L or V2075I |
| 2097 | N2097H + V2075L or V2075I |
| 2097 | N2097D + V2075L or V2075I |
| 2097 | N2097A + V2075L or V2075I |
| 2097 | N2097G + V2075L or V2075I |
| 2097 | N2097C + V2075L or V2075I |
| 2097 | N2097V + V2075L or V2075I |
| 2097 | N2097P + V2075L or V2075I |
| 2097 | N2097L + V2075L or V2075I |
| 2097 | N2097I + V2075L or V2075I |
| 2097 | N2097M + V2075L or V2075I |
| 2097 | N2097F + V2075L or V2075I |
| 2097 | N2097W + V2075L or V2075I |
| 2097 | N2097Q + V2075L or V2075I |
| 2097 | N2097S + V2075L or V2075I |
| 2097 | N2097T + V2075L or V2075I |
| 2097 | N2097Y + V2075L or V2075I |
| 2097 | N2097R + D2078G |
| 2097 | N2097K + D2078G |
| 2097 | N2097E + D2078G |
| 2097 | N2097H + D2078G |
| 2097 | N2097D + D2078G |
| 2097 | N2097A + D2078G |
| 2097 | N2097G + D2078G |
| 2097 | N2097C + D2078G |
| 2097 | N2097V + D2078G |
| 2097 | N2097P + D2078G |
| 2097 | N2097L + D2078G |
| 2097 | N2097I + D2078G |
| 2097 | N2097M + D2078G |
| 2097 | N2097F + D2078G |
| 2097 | N2097W + D2078G |
| 2097 | N2097Q + D2078G |
| 2097 | N2097S + D2078G |
| 2097 | N2097T + D2078G |
| 2097 | N2097Y + D2078G |
| 2097 | N2097R + V2098A or V2098G |
| 2097 | N2097K + V2098A or V2098G |
| 2097 | N2097E + V2098A or V2098G |
| 2097 | N2097H + V2098A or V2098G |
| 2097 | N2097D + V2098A or V2098G |
| 2097 | N2097A + V2098A or V2098G |
| 2097 | N2097G + V2098A or V2098G |
| 2097 | N2097C + V2098A or V2098G |
| 2097 | N2097V + V2098A or V2098G |
| 2097 | N2097P + V2098A or V2098G |
| 2097 | N2097L + V2098A or V2098G |
| 2097 | N2097I + V2098A or V2098G |
| 2097 | N2097M + V2098A or V2098G |
| 2097 | N2097F + V2098A or V2098G |
| 2097 | N2097W + V2098A or V2098G |
| 2097 | N2097Q + V2098A or V2098G |
| 2097 | N2097S + V2098A or V2098G |
| 2097 | N2097T + V2098A or V2098G |
| 2097 | N2097Y + V2098A or V2098G |
| 2097 | N2097S + D2078G + H2223R + S2276F |
| 2098 | V → A |
| 2098 | V → H |
| 2098 | V → P |
| 2098 | V → S |
| 2098 | V → G |
| 2098 | V → C |
| 2098 | V2098G + I1781L |
| 2098 | V2098A + A1785G |
| 2098 | V2098G + N2087S |
| 2098 | V2098A + N2087S |
| 2098 | V2098G + A1785G |
| 2098 | V2098A + W1999G |
| 2098 | V2098C + C2088W |
| 2098 | V2098A + C2088F/H/K/L/T |
| 2098 | V2098G + C2088G/H/L/S/T/V |
| 2098 | V2098A + G2096A |
| 2098 | V2098A + A2151T + V2254I |
| 2098 | V2098A + V2049A/C/L/S/T |
| 2098 | V2098G + A2226S |
| 2112 | E → G |
| 2112 | E → V |
| 2112 | E2112G + D2078G |
| 2112 | E2112V + D2078G |
| 2127 | D → N |
| 2127 | D2127N + I1766V + D2078G |
| 2149 | I → K |
| 2149 | I → V |
| 2149 | I2149K + D2078G |
| 2150 | E → K |
| 2150 | E2150K + D2078G + 12267V |
| 2154 | K → R |
| 2154 | K2154R + D2078G + K2232N |
| 2155 | Q → L |
| 2155 | Q2155L + R2085S + D2078G |
| 2169 | E → D |
| 2169 | V2075L + K1930N + A1752T + F1683L + E2169D |
| 2179 | A → T |
| 2179 | A2179T + E1946V + D2078G + F2253I |
| 2182 | V → A |
| 2182 | V2182A + V1966L + D2078G + K2317T |
| 2223 | H → R |
| 2223 | H2223R + D2078G + N2097S + S2276F |
| 2232 | K → N |
| 2232 | K2232N + D2078G + K2154R |
| 2241 | A → T |
| 2253 | F → I |
| 2253 | F2253I + A2179T + E1946V + D2078G |
| 2262 | N → K |
| 2262 | N2262K + D2078G |
| 2264 | K → R |
| 2267 | I → V |
| 2267 | I2267V + D2078G + N2097S + H2223R |
| 2276 | S → P |
| 2276 | S → F |
| 2276 | S2276P + D2078G + K2302Q |
| 2276 | S2276F + H2223R + D2078G + N2097S |
| 2280 | S → P |
| 2280 | A2059V + D2078G + S2280P + S2297C |
| 2297 | S → C |
| 2297 | S2297C + A2059V + D2078G + S2280P |
| 2302 | K → Q |
| 2302 | K2302Q + D2078G + S2276P |
| 2317 | K → T |
| 2317 | K2317T + V1966L + D2078G + V2182A |

*G1605-6 denotes a residue found in the rice ACCase enzymes but not in the blackgrass (Alopecurus myosuroides) reference sequence. The rice G1605-6 residue aligns between positions 1605 and 1606 of the blackgrass (Alopecurus myosuroides) enzyme.

In various aspects and embodiments of the present disclosure, provided include a plant having one or more ACCase mutations and/or mutation combinations selected from the group consisting of the mutations/combinations listed on Table 2 below and/or methods of making such a plant.

TABLE 2
Exemplary mutations relative to blackgrass (Alopecurus
myosuroides) plastidal ACCase positions (FIG. 1B; SEQ ID NO. 2)

| Mutation(s) | Alternative Substitution(s) |
|---|---|
| L1543F + D1681G | L1543V/R/K/E/H/D/A/G/C/P/I/M/W/Q/T/Y/S/N; |
| | D1681E/V/L/F/R/K/H/A/C/P/I/M/W/Q/T/Y/S/N |
| A1785G + I1781L | A1785R/K/E/H/D/C/V/P/L/I/M/F/W/Q/T/Y/S/N; |
| | I1781A/M/N/S/T/V |
| A1785G + C2088M | A1785R/K/E/H/D/C/V/P/L/I/M/F/W/Q/T/Y/S/N; |
| | C2088F/G/H/K/L/P/Q/R/S/T/V/W/N |
| L1818H + S1784N | L1818I/R/K/E/D/A/G/C/V/P/M/F/W/Q/T/Y/S/N; |
| | S1784G/R/K/E/H/D/A/C/V/P/L/I/M/F/W/Q/T/Y |
| L1818H + V1529M | L1818I/R/K/E/D/A/G/C/V/P/M/F/W/Q/T/Y/S/N; |
| | V1529R/K/E/H/D/A/G/C/P/L/I/F/W/Q/T/Y/S/N |
| L1818H + K1840R | L1818I/R/K/E/D/A/G/C/V/P/M/F/W/Q/T/Y/S/N; |
| | K1840M/E/H/D/A/G/C/V/P/L/I/F/W/Q/T/Y/S/N |
| V2075L + N2097S | V2075I; |
| | N2097R/K/E/H/D/A/G/C/V/P/L/I/M/F/W/Q/T/Y |
| V2075L + I1781T | V2075I; |
| | I1781A/M/N/S/L/V |
| D2078G + N2097S | D2078K/T; |
| | N2097R/K/E/H/D/A/G/C/V/P/L/I/M/F/W/Q/T/Y |
| D2078G + P2083T | D2078K/T; |
| | P2083N/Q/S |
| D2078G + C2088N | D2078K/T; |
| | C2088F/G/H/K/L/P/Q/R/S/T/V/W/M |
| D2078G + R1672I | D2078K/T; |
| | R1672K/E/H/D/A/G/C/V/P/L/M/F/W/Q/T/Y/S/N |
| D2078G + M1974T | D2078K/T; |
| | M1974R/K/E/H/D/A/G/C/V/P/L/I/F/W/Q/Y/S/N |
| V2098A + N2097S | N2097R/K/E/H/D/A/G/C/V/P/L/I/M/F/W/Q/T/Y |
| V2098G + N2097S | N2097R/K/E/H/D/A/G/C/V/P/L/I/M/F/W/Q/T/Y |
| V1529M | V1529R/K/E/H/D/A/G/C/P/L/I/F/W/Q/T/Y/S/N |
| L1543F | L1543V/R/K/E/H/D/A/G/C/P/I/M/W/Q/T/Y/S/N |
| D1681G | D1681E/V/L/F/R/K/H/A/C/P/I/M/W/Q/T/Y/S/N |
| L1818H | L1818I/R/K/E/D/A/G/C/V/P/M/F/W/Q/T/Y/S/N |
| K1840R | K1840M/E/H/D/A/G/C/V/P/L/I/F/W/Q/T/Y/S/N |
| R1672I | R1672K/E/H/D/A/G/C/V/P/L/M/F/W/Q/T/Y/S/N |
| I1781L | I1781A/M/N/S/T/V |
| I1781T | I1781A/M/N/S/L/V |
| S1784N | S1784G/R/K/E/H/D/A/C/V/P/L/I/M/F/W/Q/T/Y |
| A1785G | A1785R/K/E/H/D/C/V/P/L/I/M/F/W/Q/T/Y/S/N |
| M1974T | M1974R/K/E/H/D/A/G/C/V/P/L/I/F/W/Q/Y/S/N |
| V2075L | V2075I |
| D2078G | D2078K/T |
| P2083T | P2083N/Q/S |
| C2088N | C2088R/K/E/H/D/A/G/V/P/L/I/M/F/W/Q/T/Y/S |
| N2097S | N2097R/K/E/H/D/A/G/C/V/P/L/I/M/F/W/Q/T/Y |
| V2098A | V2098G |

In various aspects and embodiments of the present disclosure, provided include a plant having one or more ACCase mutations and/or mutation combinations selected from the group consisting of the mutations/combinations listed on Table 3 below and/or methods of making such a plant.

TABLE 3
Exemplary mutations relative to blackgrass (Alopecurus myosuroides)
plastidal ACCase positions (FIG. 1B; SEQ ID NO. 2)
Mutation(s)

V1529M + L1818H
L1555P + S1605Y + L1985P + D2078G
C1575S + G1605-6A* + V2075L
S1602F + D1725E + I1781T + V2075L
S1603N + L1649M + V1715A + D2078G
S1605Y + V2098A
A1957T + V2075L
V1966L + D2078G + V2182A + A2244T + K2317T
I1967V + V2075L + S2119C
R1672I + M1975T + D2078G
R1672I + D2078G
F1683L + A1752T + K1930N + V2075L + E2169D
I1766V + D2078G + D2127N
K1772E + S1784N + V1924D
E1946V + D2078G + A2179T + F2253I
M1974T + D2078G + M2257I
M1974T + D2078G
M1975T + D2078G
V1992D + V2075L + F2196Y + L2299I
V2049I + V2075L + A2178I
A2059V + D2078G + S2280P + S2297C
A2059V + D2078G + S2280P + S2297C
M2065K + D2078G + R2085S + Q2155L + Q2220L
A2067S + V2075L + L2159P
V2075L + N2097S
D2078G
D2078G + P2083T
D2078G + N2097S + H2223R + S2276F
D2078G + E2112V
D2078G + I2149K
D2078G + E2150K + I2267V
D2078G + K2154R + K2232N
D2078G + M2257I
D2078G + N2262K
D2078G + S2276P + K2302Q
V2098A + V2134I + A2151T + V2254I
V2098G
V2098G + A2226S

In some embodiments the plant or plant cell includes a mutated ACCase gene, wherein the gene encodes a protein including a mutation at one or more amino acid positions corresponding to a position or position combinations selected from the group consisting of L1543V/D1681E; L1543F/D1681E; L1543V/D1681G; L1543F/D1681G; A1785G/I1781L; A1785G/I1781T; A1785G/I1781V; A1785G/C2088M; A1785G/C2088N; A1785G/C2088N; A1785G/C2088R; L1818H/S1784N; L1818H/S1784G; L1818I/S1784N; L1818I/S1784G; V1529M/L1818H; V1529M/L1818I; K1840R/L1818H; K1840R/L1818I; K1840M/L1818H; K1840M/L1818I; N2097D/D2078G; N2097S/D2078G; N2097D/V2075L; N2097S/V2075I; N2097D/V2075I; N2097S/V2075L; N2097D/V2098A; N2097D/V2098G; N2097S/V2098A; N2097S/V2098G; R1672I/D2078G; M1974T/D2078G; D2078G/P2083T; D2078G/C2088M; D2078G/C2088N; D2078G/C2088R; K1840R/L1818H; S1784N/L1818H, D1725E/I1781T/V2075L; I1781T/V2075L; I1781L/V2075I; I1781L/V2075L; I1781T/V2075I; and I1781V/V2075I; I1781V/V2075L; I1781V/V2075I; and I1781V/V2075L of SEQ ID NO: 2.

In certain embodiments, a plant or plant cell may include a mutated ACCase gene wherein the gene encodes an ACCase protein including a mutation at an amino acid position(s) corresponding to L1543V/D1681E of SEQ ID NO: 2. In certain embodiments, a plant or plant cell may include a mutated ACCase gene wherein the gene encodes an ACCase protein including a mutation at an amino acid position(s) corresponding to L1543F/D1681E of SEQ ID NO: 2. In certain embodiments, a plant or plant cell may include a mutated ACCase gene wherein the gene encodes an ACCase protein including a mutation at an amino acid position(s) corresponding to L1543V/D1681G of SEQ ID NO: 2. In certain embodiments, a plant or plant cell may include a mutated ACCase gene wherein the gene encodes an ACCase protein including a mutation at an amino acid position(s) corresponding to L1543F/D1681G of SEQ ID NO: 2. In certain embodiments, a plant or plant cell may include a mutated ACCase gene wherein the gene encodes an ACCase protein including a mutation at an amino acid position(s) corresponding to A1785G/I1781L of SEQ ID NO: 2. In certain embodiments, a plant or plant cell may include a mutated ACCase gene wherein the gene encodes an ACCase protein including a mutation at an amino acid position(s) corresponding to A1785G/I1781T of SEQ ID NO: 2. In certain embodiments, a plant or plant cell may include a mutated ACCase gene wherein the gene encodes an ACCase protein including a mutation at an amino acid position(s) corresponding to A1785G/I1781V of SEQ ID NO: 2. In certain embodiments, a plant or plant cell may include a mutated ACCase gene wherein the gene encodes an ACCase protein including a mutation at an amino acid position(s) corresponding to A1785G/C2088M of SEQ ID NO: 2. In certain embodiments, a plant or plant cell may include a mutated ACCase gene wherein the gene encodes an ACCase protein including a mutation at an amino acid position(s) corresponding to A1785G/C2088M of SEQ ID NO: 2. In certain embodiments, a plant or plant cell may include a mutated ACCase gene wherein the gene encodes an ACCase protein including a mutation at an amino acid position(s) corresponding to A1785G/C2088N of SEQ ID NO: 2. In certain embodiments, a plant or plant cell may include a mutated ACCase gene wherein the gene encodes an ACCase protein including a mutation at an amino acid position(s) corresponding to A1785G/C2088R of SEQ ID NO: 2. In certain embodiments, a plant or plant cell may include a mutated ACCase gene wherein the gene encodes an ACCase protein including a mutation at an amino acid position(s) corresponding to L1818H/S1784N of SEQ ID NO: 2. In certain embodiments, a plant or plant cell may include a mutated ACCase gene wherein the gene encodes an ACCase protein including a mutation at an amino acid position(s) corresponding to L1818H/S1784G of SEQ ID NO: 2. In certain embodiments, a plant or plant cell may include a mutated ACCase gene wherein the gene encodes an ACCase protein including a mutation at an amino acid position(s) corresponding to L1818I/S1784N of SEQ ID NO: 2. In certain embodiments, a plant or plant cell may include a mutated ACCase gene wherein the gene encodes an ACCase protein including a mutation at an amino acid position(s) corresponding to L1818I/S1784G of SEQ ID NO: 2. In certain embodiments, a plant or plant cell may include a mutated ACCase gene wherein the gene encodes an ACCase protein including a mutation at an amino acid position(s) corresponding to V1529M/L1818H of SEQ ID NO: 2. In certain embodiments, a plant or plant cell may include a mutated ACCase gene wherein the gene encodes an ACCase protein including a mutation at an amino acid position(s) corresponding to V1529M/L1818I of SEQ ID NO: 2. In certain embodiments, a plant or plant cell may include a mutated ACCase gene wherein the gene encodes an ACCase protein including a mutation at an amino acid position(s) corresponding to K1840R/L1818H of SEQ ID NO: 2. In certain embodiments, a plant or plant cell may include a mutated ACCase gene wherein the gene encodes an ACCase protein including a mutation at an amino acid position(s) corresponding to K1840R/L1818I of SEQ ID NO: 2. In certain embodiments, a plant or plant cell may include a mutated ACCase gene wherein the gene encodes an ACCase protein including a mutation at an amino acid position(s) corresponding to K1840M/L1818H of SEQ ID NO: 2. In certain embodiments, a plant or plant cell may include a mutated ACCase gene wherein the gene encodes an ACCase protein including a mutation at an amino acid position(s) corresponding to K1840M/L1818I of SEQ ID NO: 2. In certain embodiments, a plant or plant cell may include a mutated ACCase gene wherein the gene encodes an ACCase protein including a mutation at an amino acid position(s) corresponding to N2097S/D2078G of SEQ ID NO: 2. In certain embodiments, a plant or plant cell may include a mutated ACCase gene wherein the gene encodes an ACCase protein including a mutation at an amino acid position(s) corresponding to N2097S/V2075L of SEQ ID NO: 2. In certain embodiments, a plant or plant cell may include a mutated ACCase gene wherein the gene encodes an ACCase protein including a mutation at an amino acid position(s) corresponding to N2097S/V2098A of SEQ ID NO: 2. In certain embodiments, a plant or plant cell may include a mutated ACCase gene wherein the gene encodes an ACCase protein including a mutation at an amino acid position(s) corresponding to N2097S/V2098G of SEQ ID NO: 2. In certain embodiments, a plant or plant cell may include a mutated ACCase gene wherein the gene encodes an ACCase protein including a mutation at an amino acid position(s) corresponding to R1672I/D2078G of SEQ ID NO: 2. In certain embodiments, a plant or plant cell may include a mutated ACCase gene wherein the gene encodes an ACCase protein including a mutation at an amino acid position(s) corresponding to M1974T/D2078G of SEQ ID NO: 2. In certain embodiments, a plant or plant cell may include a mutated ACCase gene wherein the gene encodes an ACCase protein including a mutation at an amino acid position(s) corresponding to D2078G/P2083T of SEQ ID NO: 2. In certain embodiments, a plant or plant cell may include a mutated ACCase gene wherein the gene encodes an ACCase protein including a mutation at an amino acid position(s) corresponding to D2078G/C2088N of SEQ ID NO: 2. In certain embodiments, a plant or plant cell may include a mutated ACCase gene wherein the gene encodes an ACCase protein including a mutation at an amino acid position(s) corresponding to K1840R/L1818H of SEQ ID NO: 2. In certain embodiments, a plant or plant cell may include a mutated ACCase gene wherein the gene encodes an ACCase protein including a mutation at an amino acid position(s) corresponding to S1784N/L1818H of SEQ ID NO: 2. In certain embodiments, a plant or plant cell may include a mutated ACCase gene wherein the gene encodes an ACCase protein including a mutation at an amino acid position(s) corresponding to S1784N/L1818H of SEQ ID NO: 2. In certain embodiments, a plant or plant cell may include a mutated ACCase gene wherein the gene encodes an ACCase protein including a mutation at an amino acid position(s) corresponding to D1725E/I1781T/V2075L of SEQ ID NO: 2. In certain embodiments, a plant or plant cell may include a mutated ACCase gene wherein the gene encodes an ACCase protein including a mutation at an amino acid position(s) corresponding to I1781T/V2075L of SEQ ID NO: 2. In certain embodiments, a plant or plant cell may include a mutated ACCase gene wherein the gene encodes an ACCase protein including a mutation at an amino acid position(s) corresponding to I1781L/V2075I of SEQ ID NO: 2. In certain embodiments, a plant or plant cell may include a mutated ACCase gene wherein the gene encodes an ACCase protein including a mutation at an amino acid position(s) corresponding to I1781L/V2075L of SEQ ID NO: 2. In certain embodiments, a plant or plant cell may include a mutated ACCase gene wherein the gene encodes an ACCase protein including a mutation at an amino acid position(s) corresponding to I1781T/V2075I of SEQ ID NO: 2. In certain embodiments, a plant or plant cell may include a mutated ACCase gene wherein the gene encodes an ACCase protein including a mutation at an amino acid position(s) corresponding to I1781V/V2075I of SEQ ID NO: 2. In certain embodiments, a plant or plant cell may include a mutated ACCase gene wherein the gene encodes an ACCase protein including a mutation at an amino acid position(s) corresponding to I1781V/V2075L of SEQ ID NO: 2.

In some embodiments the plant or plant cell includes a mutated ACCase gene, wherein the gene encodes a protein including a mutation at one or more amino acid positions corresponding to a position or position combinations selected from the group consisting of D1725E/I1781T/V2075L, V2098G, A2059V/D2078G/S2280P/S2297C, E2112V/D2078G, D1681G, D2078G/R1672I/M1975T, H2223R/D2078G/N2097S/S2276F, C1575S/G1605-6A*/V2075L, D2078G/S2276P/K2302Q, R2085S/D2078G/Q2155L, M1974T/D2078G, D2078G/I2267V/E2150K, K1930N/A1752T/F1683L/V2075L/E2169D, I1967V/V2075L, K1772E/S1784G, K2154R/D2078G/K2232N, I2149K/D2078G, A2179T/E1946V/D2078G/F2253I, S1605Y/V2098A, V1529M/L1818H, V1992D/V2075L/F2196Y/L2299I, V2049I/V2075L/A2178I, V2075L/N2097S, V2098A/A2151T/V2254I, L1649M/V1715A/D2078G, L1555P/S1605Y/D20786G, I1766V/D2078G/D2127N, D2078G, D2078G/P2083T, V1966L/D2078G/V2182A/K2317T, A2067S/V2075L/L2159P, V2098G/A2226S, A1957T/V2075L, and N2262K/D2078G of SEQ ID NO: 2.

In certain embodiments, a plant or plant cell may include a mutated ACCase gene wherein the gene encodes an ACCase protein including a mutation at an amino acid position(s) corresponding to D1725E/I1781T/V2075L of SEQ ID NO: 2. In certain embodiments, a plant or plant cell may include a mutated ACCase gene wherein the gene encodes an ACCase protein including a mutation at an amino acid position(s) corresponding to V2098G of SEQ ID NO: 2. In certain embodiments, a plant or plant cell may include a mutated ACCase gene wherein the gene encodes an ACCase protein including a mutation at an amino acid position(s) corresponding to A2059V/D2078G/S2280P/S2297C of SEQ ID NO: 2. In certain embodiments, a plant or plant cell may include a mutated ACCase gene wherein the gene encodes an ACCase protein including a mutation at an amino acid position(s) corresponding to E2112V/D2078G of SEQ ID NO: 2. In certain embodiments, a plant or plant cell may include a mutated ACCase gene wherein the gene encodes an ACCase protein including a mutation at an amino acid position(s) corresponding to D1681G of SEQ ID NO: 2. In certain embodiments, a plant or plant cell may include a mutated ACCase gene wherein the gene encodes an ACCase protein including a mutation at an amino acid position(s) corresponding to D2078G/R1672I/M1975T of SEQ ID NO: 2. In certain embodiments, a plant or plant cell may include a mutated ACCase gene wherein the gene encodes an ACCase protein including a mutation at an amino acid position(s) corresponding to H2223R/D2078G/N2097S/S2276F of SEQ ID NO: 2. In certain embodiments, a plant or plant cell may include a mutated ACCase gene wherein the gene encodes an ACCase protein including a mutation at an amino acid position(s) corresponding to C1575S/G1605-6A*/V2075L of SEQ ID NO: 2. In certain embodiments, a plant or plant cell may include a mutated ACCase gene wherein the gene encodes an ACCase protein including a mutation at an amino acid position(s) corresponding to D2078G/S2276P/K2302Q of SEQ ID NO: 2. In certain embodiments, a plant or plant cell may include a mutated ACCase gene wherein the gene encodes an ACCase protein including a mutation at an amino acid position(s) corresponding to R2085S/D2078G/Q2155L of SEQ ID NO: 2. In certain embodiments, a plant or plant cell may include a mutated ACCase gene wherein the gene encodes an ACCase protein including a mutation at an amino acid position(s) corresponding to M1974T/D2078G of SEQ ID NO: 2. In certain embodiments, a plant or plant cell may include a mutated ACCase gene wherein the gene encodes an ACCase protein including a mutation at an amino acid position(s) corresponding to D2078G/12267V/E2150K of SEQ ID NO: 2. In certain embodiments, a plant or plant cell may include a mutated ACCase gene wherein the gene encodes an ACCase protein including a mutation at an amino acid position(s) corresponding to K1930N/A1752T/F1683L/V2075L/E2169D of SEQ ID NO: 2. In certain embodiments, a plant or plant cell may include a mutated ACCase gene wherein the gene encodes an ACCase protein including a mutation at an amino acid position(s) corresponding to I1967V/V2075L of SEQ ID NO: 2. In certain embodiments, a plant or plant cell may include a mutated ACCase gene wherein the gene encodes an ACCase protein including a mutation at an amino acid position(s) corresponding to K1772E/S1784G of SEQ ID NO: 2. In certain embodiments, a plant or plant cell may include a mutated ACCase gene wherein the gene encodes an ACCase protein including a mutation at an amino acid position(s) corresponding to K2154R/D2078G/K2232N of SEQ ID NO: 2. In certain embodiments, a plant or plant cell may include a mutated ACCase gene wherein the gene encodes an ACCase protein including a mutation at an amino acid position(s) corresponding to I2149K/D2078G of SEQ ID NO: 2. In certain embodiments, a plant or plant cell may include a mutated ACCase gene wherein the gene encodes an ACCase protein including a mutation at an amino acid position(s) corresponding to A2179T/E1946V/D2078G/F2253I of SEQ ID NO: 2. In certain embodiments, a plant or plant cell may include a mutated ACCase gene wherein the gene encodes an ACCase protein including a mutation at an amino acid position(s) corresponding to S1605Y/V2098A of SEQ ID NO: 2. In certain embodiments, a plant or plant cell may include a mutated ACCase gene wherein the gene encodes an ACCase protein including a mutation at an amino acid position(s) corresponding to V1529M/L1818H of SEQ ID NO: 2. In certain embodiments, a plant or plant cell may include a mutated ACCase gene wherein the gene encodes an ACCase protein including a mutation at an amino acid position(s) corresponding to V1992D/V2075L/F2196Y/L22991 of SEQ ID NO: 2. In certain embodiments, a plant or plant cell may include a mutated ACCase gene wherein the gene encodes an ACCase protein including a mutation at an amino acid position(s) corresponding to V2049I/V2075L/A2178I of SEQ ID NO: 2. In certain embodiments, a plant or plant cell may include a mutated ACCase gene wherein the gene encodes an ACCase protein including a mutation at an amino acid position(s) corresponding to V2075L/N2097S of SEQ ID NO: 2. In certain embodiments, a plant or plant cell may include a mutated ACCase gene wherein the gene encodes an ACCase protein including a mutation at an amino acid position(s) corresponding to V2098A/A2151T/V2254I of SEQ ID NO: 2. In certain embodiments, a plant or plant cell may include a mutated ACCase gene wherein the gene encodes an ACCase protein including a mutation at an amino acid position(s) corresponding to L1649M/V1715A/D2078G of SEQ ID NO: 2. In certain embodiments, a plant or plant cell may include a mutated ACCase gene wherein the gene encodes an ACCase protein including a mutation at an amino acid position(s) corresponding to L1555P/S1605Y/D20786G of SEQ ID NO: 2. In certain embodiments, a plant or plant cell may include a mutated ACCase gene wherein the gene encodes an ACCase protein including a mutation at an amino acid position(s) corresponding to I1766V/D2078G/D2127N of SEQ ID NO: 2. In certain embodiments, a plant or plant cell may include a mutated ACCase gene wherein the gene encodes an ACCase protein including a mutation at an amino acid position(s) corresponding to D2078G of SEQ ID NO: 2. In certain embodiments, a plant or plant cell may include a mutated ACCase gene wherein the gene encodes an ACCase protein including a mutation at an amino acid position(s) corresponding to V1966L/D2078G/V2182A/K2317T of SEQ ID NO: 2. In certain embodiments, a plant or plant cell may include a mutated ACCase gene wherein the gene encodes an ACCase protein including a mutation at an amino acid position(s) corresponding to A2067S/V2075L/L2159P of SEQ ID NO: 2. In certain embodiments, a plant or plant cell may include a mutated ACCase gene wherein the gene encodes an ACCase protein including a mutation at an amino acid position(s) corresponding to V2098G/A2226S of SEQ ID NO: 2. In certain embodiments, a plant or plant cell may include a mutated ACCase gene wherein the gene encodes an ACCase protein including a mutation at an amino acid position(s) corresponding to A1957T/V2075L of SEQ ID NO: 2. In certain embodiments, a plant or plant cell may include a mutated ACCase gene wherein the gene encodes an ACCase protein including a mutation at an amino acid position(s) corresponding to N2262K/D2078G of SEQ ID NO: 2.

In some embodiments provided is a plant or a plant cell having an ACCase gene with one or more mutations wherein A1785G enhances the herbicide tolerance of an I1781L mutation. In some embodiments provided is a plant or a plant cell having an ACCase gene with one or more mutations wherein A1785G enhances the herbicide tolerance of an S1784G mutation. In some embodiments provided is a plant or a plant cell having an ACCase gene with one or more mutations wherein A1785G enhances the herbicide tolerance of an S1784N mutation. In some embodiments provided is a plant or a plant cell having an ACCase gene with one or more mutations wherein an A1785G mutation enhances the herbicide tolerance of a D2078G mutation. In some embodiments provided is a plant or a plant cell having an ACCase gene with one or more mutations wherein an A1785G mutation enhances the herbicide tolerance of an C2088M mutation. In some embodiments provided is a plant or a plant cell having an ACCase gene with one or more mutations wherein an N2097S mutation enhances the herbicide tolerance of a D2078G mutation. In some embodiments provided is a plant or a plant cell having an ACCase gene with one or more mutations wherein a N2097S mutation enhances the herbicide tolerance of a V2075L mutation. In some embodiments provided is a plant or a plant cell having an ACCase gene with one or more mutations wherein a N2097S mutation enhances the herbicide tolerance of a V2098A mutation. In some embodiments provided is a plant or a plant cell having an ACCase gene with one or more mutations wherein a N2097S mutation enhances the herbicide tolerance of a V2098G mutation. In some embodiments provided is a plant or a plant cell having an ACCase gene with one or more mutations wherein a R1672I mutation enhances the herbicide tolerance of a D2078G mutation. In some embodiments provided is a plant or a plant cell having an ACCase gene with one or more mutations wherein a M1974T mutation enhances the herbicide tolerance of a D2078G mutation. In some embodiments provided is a plant or a plant cell having an ACCase gene with one or more mutations wherein a P2083T mutation enhances the herbicide tolerance of a D2078G mutation. In some embodiments provided is a plant or a plant cell having an ACCase gene with one or more mutations wherein a K1840R mutation enhances the herbicide tolerance of a L1818H mutation. In some embodiments provided is a plant or a plant cell having an ACCase gene with one or more mutations wherein an I1781T mutation enhances the herbicide tolerance of a V2075L mutation. In each case, the numbering of the ACCase is relative to SEQ ID NO: 2

In certain embodiments, a plant or plant cell comprises at least two mutations in an ACCase gene such that the mutated gene encodes a protein comprising a first substitution at a position corresponding to D2078 of SEQ ID NO: 2 and a second substitution at a position corresponding to P2083 of SEQ ID NO: 2. Preferably, the plant or plant cell is not transgenic with respect to the D2078/P2083 substitutions (e.g., the mutations creating these substitutions are introduced through a gene editing procedure). As noted herein, SEQ ID NO: 2 is used herein as a reference sequence for other homologous ACCase sequences. Thus, the phrase “substitution corresponding to <amino acid substitution at a specified location> in SEQ ID NO: 2” as used herein refers to the same substitution at a corresponding amino acid location in a homologous ACCase protein sequence. By way of example, “an ACCase protein comprising a substitution corresponding to D2078G of SEQ ID NO: 2” includes a D2076G substitution in SEQ ID NO: 6 (Oryza sativa subsp. Japonica ACCase) and a D2083G substitution in SEQ ID NO: 22 (Sorghum bicolor). Exemplary D2078/P2083 amino acid positions compared across species are shown, but not limited to, those listed below:

| | | Substi- | Substi- |
|---|---|---|---|
| | | tution 1 | tution 2 |
| Sequence No. | Species | Position | Position |
| 1 (SEQ ID NO: 2) | Alopecurus myosuroides | D2078 | P2083 |
| 2 (SEQ ID NO: 970) | Avena sativa | D2078 | P2083 |
| 3 (SEQ ID NO: 972) | Avena sativa | D2078 | P2083 |
| 4 (SEQ ID NO: 974) | Avena sativa | D2078 | P2083 |
| 5 (SEQ ID NO: 23) | Brachypodium | D2090 | P2095 |
| | distachyon | | |
| 6 (SEQ ID NO: 956) | Hordeum vulgare subsp. | D2066 | P2071 |
| | vulgare | | |
| 7 (SEQ ID NO: 966) | Lolium perenne | D2076 | P2081 |
| 8 (SEQ ID NO: 954) | Oryza sativa subsp. | D2089 | P2094 |
| | Japonica | | |
| 9 (SEQ ID NO: 6) | Oryza sativa subsp. | D2076 | P2081 |
| | Japonica | | |
| 10 (SEQ ID NO: 4) | Oryza sativa cultivar | D2089 | P2094 |
| | Cypress | | |
| 11 (SEQ ID NO: 976) | Poa pratensis | D2078 | P2083 |
| 12 (SEQ ID NO: 978) | Poa pratensis | D2078 | P2083 |
| 13 (SEQ ID NO: 968) | Panicum virgatum | D2077 | P2082 |
| 14 (SEQ ID NO: 22) | Sorghum bicolor | D2083 | P2088 |
| 15 (SEQ ID NO: 953) | Sorghum bicolor | D2083 | P2088 |
| 16 (SEQ ID NO: 16) | Setaria italica | D2077 | P2082 |
| 17 (SEQ ID NO: 18) | Setaria italica | D2077 | P2082 |
| 18 (SEQ ID NO: 20) | Setaria italica | D2077 | P2082 |
| 19 (SEQ ID NO: 964) | Setaria italica | D2078 | P2083 |
| 20 (SEQ ID NO: 8) | Triticum aestivum | D2066 | P2071 |
| 21 (SEQ ID NO: 958) | Triticum aestivum | D2066 | P2071 |
| 22 (SEQ ID NO: 960) | Triticum aestivum | D2066 | P2071 |
| 23 (SEQ ID NO: 962) | Triticum aestivum | D2066 | P2071 |
| 24 (SEQ ID NO: 10) | Zea mays | D2080 | P2085 |
| 25 (SEQ ID NO: 12) | Zea mays | D2081 | P2086 |
| 26 (SEQ ID NO: 14) | Zea mays | D2081 | P2086 |
| 27 (SEQ ID NO: 951) | Zea mays | D2081 | P2086 |

The term “ACCase homolog” or any variation therefore refers to an ACCase gene or ACCase gene product found in the same or another species that performs the same or substantially the same biological function as the blackgrass (Alopecurus myosuroides) ACCase genes and gene products disclosed herein and where the nucleic acid sequences or polypeptide sequences (of the ACCase gene product) are said to be “identical” or at least 50% similar (also referred to as “percent identity” or “substantially identical”) as described herein.

### Methods of Identifying Sequence Similarity

Two polynucleotides or polypeptides are identical if the sequence of nucleotides or amino acid residues, respectively, in the two sequences is the same when aligned for maximum correspondence as described below. The terms “identical” or “percent identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. For polypeptides where sequences differ in conservative substitutions, the percent sequence identity may be adjusted upwards to correct for the conservative nature of the substitution. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., U.S.A.).

The phrases “substantially identical,” and “percent identity” in the context of two nucleic acids or polypeptides, refer to sequences or subsequences that have at least 50%, advantageously 60%, preferably 70%, more preferably 80%, and most preferably 90-95% nucleotide or amino acid residue identity when aligned for maximum correspondence over a comparison window as measured using one of the following sequence comparison algorithms or by manual alignment and visual inspection. This definition also refers to the complement of a test sequence, which has substantial sequence or subsequence complementarity when the test sequence has substantial identity to a reference sequence.

One of skill in the art will recognize that two polypeptides can also be “substantially identical” if the two polypeptides are immunologically similar. Thus, overall protein structure may be similar while the primary structure of the two polypeptides displays significant variation. Therefore, a method to measure whether two polypeptides are substantially identical involves measuring the binding of monoclonal or polyclonal antibodies to each polypeptide. Two polypeptides are substantially identical if the antibodies specific for a first polypeptide bind to a second polypeptide with an affinity of at least one third of the affinity for the first polypeptide. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, 0.4dv. Appl. Math. 2:482 (I 98 I), by the homology alignment algorithm of Needleman & Wunsch, J. Mol. Biol. 48:443 (1970), by the search for similarity method of Pearson & Lipman, Proc. Nat'l. Acad. Sci. U.S.A. 5 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), by software for alignments such as VECTOR NTI Version #11.5 by Life Technologies, Carlsbad, CA, U.S.A., by the procedures described in ClustalW, Thompson, J. D., Higgins, D. G. and Gibson, T. J. (1994) CLUSTALW: improving the sensitivity of progressive multiple sequence alignment through sequence weighting, position-specific gap penalties and weight matrix choice. Nucleic Acids Research, 22:4673-4680 or by visual inspection (see generally, Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:33 89-3402, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. U.S.A. 89:10915 (1989)). In addition to calculating percent sequence identity, the BLAST algorithm also performs a statistical analysis of the similarity between two sequences (see, e.g., Karlin & Altschul, Proc. Nat'l. Acad. Sci. U.S.A. 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### Nucleic Acids and Delivery Thereof to Cells

Certain aspects of the present disclosure involve nucleic acids (e.g., ACCase genes), as well as nucleic acids having one or more mutations. Various methods exist for inducing mutations in a nucleic acid, as described herein. In some embodiments, one or more nucleic acids may be delivered to a cell, as described herein.

### Oligonucleobases

As used herein, an “oligonucleobase” is a polymer of nucleobases, which polymer can hybridize by Watson-Crick base pairing to a DNA having the complementary sequence.

Nucleobases comprise a base, which may be a purine, pyrimidine, or a derivative or analog thereof. Nucleobases include peptide nucleobases, the subunits of peptide nucleic acids, and morpholine nucleobases as well as nucleosides and nucleotides. Nucleosides are nucleobases that contain a pentosefuranosyl moiety, e.g., an optionally substituted riboside or 2′-deoxyriboside. Nucleosides can be linked by one of several linkage moieties, which may or may not contain phosphorus. Nucleosides that are linked by unsubstituted phosphodiester linkages are termed nucleotides.

An oligonucleobase chain may have a single 5′ and 3′ terminus, which are the ultimate nucleobases of the polymer. A particular oligonucleobase chain can contain nucleobases of all types. An oligonucleobase compound is a compound comprising one or more oligonucleobase chains that are complementary and hybridized by Watson-Crick base pairing. Nucleobases are either deoxyribo-type or ribo-type. Ribo-type nucleobases are pentosefuranosyl containing nucleobases wherein the 2′ carbon is a methylene substituted with a hydroxyl, alkyloxy or halogen. Deoxyrib-type nucleobases are nucleobases other than ribo-type nucleobases and include all nucleobases that do not contain a pentosefuranosyl moiety.

An oligonucleobase strand generically includes both oligonucleobase chains and segments or regions of oligonucleobase chains. An oligonucleobase strand has a 3′ end and a 5′ end. When an oligonucleobase strand is coextensive with a chain, the 3′ and 5′ ends of the strand are also 3′ and 5′ termini of the chain.

The oligonucleobase can be introduced into a plant cell using any method commonly used in the art, including but not limited to, microcarriers (biolistic delivery), microfibers (whiskers), electroporation, nucleofection, PEG-mediated delivery, direct DNA uptake and microinjection. Illustrative examples of an oligonucleobase are described below.

The description can be practiced with oligonucleobases having the conformations and chemistries described in the Kmiec I and Kmiec II patents which are incorporated herein by reference. Kmiec I discloses a method for introducing specific genetic alterations into a target gene. The oligonucleobases in Kmiec I and/or Kmiec II contain two complementary strands, one of which contains at least one segment of RNA-type nucleotides (an “RNA segment”) that are base paired to DNA-type nucleotides of the other strand.

Kmiec II discloses that purine and pyrimidine base-containing non-nucleotides can be substituted for nucleotides. U.S. Pat. Nos. 5,756,325; 5,871,984; 5,760,012; 5,888,983; 5,795,972; 5,780,296; 5,945,339; 6,004,804; and 6,010,907 and in International Patent No. PCT/US00/23457; and in International Patent Publication Nos. WO 98/49350; WO 99/07865; WO 99/58723; WO 99/58702; WO 99/40789; U.S. Pat. No. 6,870,075; and U.S. Published patent application No. 20030084473, which are each hereby incorporated in their entirety, disclose additional molecules that can be used for the present description. The term “oligonucleobase” is used herein to denote the molecules that can be used in the methods of the present disclosure and include mixed duplex oligonucleotides, non-nucleotide containing molecules taught in Kmiec II, single stranded oligodeoxynucleotides and other molecules taught in the above noted patents and patent publications.

In one embodiment, the oligonucleobase is a mixed duplex oligonucleotide in which the RNA-type nucleotides of the mixed duplex oligonucleotide are made RNase resistant by replacing the 2′-hydroxyl with a fluoro, chloro or bromo functionality or by placing a substituent on the 2′-O. Suitable substituents include the substituents taught by the Kmiec II. Alternative substituents include the substituents taught by U.S. Pat. No. 5,334,711 (Sproat) and the substituents taught by patent publications EP 629 387 and EP 679 657 (collectively, the Martin Applications), which are incorporated herein by reference. As used herein, a 2′-fluoro, chloro or bromo derivative of a ribonucleotide or a ribonucleotide having a 2′-OH substituted with a substituent described in the Martin Applications or Sproat is termed a “2′-substituted ribonucleotide.” As used herein the term “RNA-type nucleotide” means a 2′-hydroxyl or 2′-substituted nucleotide that is linked to other nucleotides of a mixed duplex oligonucleotide by an unsubstituted phosphodiester linkage or any of the non-natural linkages taught by Kmiec I or Kmiec II. As used herein the term “deoxyribo-type nucleotide” means a nucleotide having a 2′-H, which can be linked to other nucleotides of a MDON by an unsubstituted phosphodiester linkage or any of the non-natural linkages taught by Kmiec I or Kmiec II.

In one embodiment of the present disclosure, the oligonucleobase or GRON is a mixed duplex oligonucleotide that is linked solely by unsubstituted phosphodiester bonds. In alternative embodiments, the linkage is by substituted phosphodiesters, phosphodiester derivatives and non-phosphorus-based linkages as taught by Kmiec II. In yet another embodiment, each RNA-type nucleotide in the mixed duplex oligonucleotide is a 2′-substituted nucleotide. Particularly preferred embodiments of 2′-substituted ribonucleotides are 2′-fluoro, 2′-methoxy, 2′-propyloxy, 2′-allyloxy, 2′-hydroxylethyloxy, 2′-methoxyethyloxy, 2′-fluoropropyloxy and 2′-trifluoropropyloxy substituted ribonucleotides. More preferred embodiments of 2′-substituted ribonucleotides are 2′-fluoro, 2′-methoxy, 2′-methoxyethyloxy, and 2′-allyloxy substituted nucleotides. In another embodiment the mixed duplex oligonucleotide is linked by unsubstituted phosphodiester bonds.

Although mixed duplex oligonucleotides having only a single type of 2′-substituted RNA-type nucleotide are more conveniently synthesized, the methods of the disclosure can be practiced with mixed duplex oligonucleotides having two or more types of RNA-type nucleotides. The function of an RNA segment may not be affected by an interruption caused by the introduction of a deoxynucleotide between two RNA-type trinucleotides, accordingly, the term RNA segment encompasses such an “interrupted RNA segment.” An uninterrupted RNA segment is termed a contiguous RNA segment. In an alternative embodiment an RNA segment can contain alternating RNase-resistant and unsubstituted 2′-OH nucleotides. The mixed duplex oligonucleotides preferably have fewer than 100 nucleotides and more preferably fewer than 85 nucleotides, but more than 50 nucleotides. The first and second strands are Watson-Crick base paired. In one embodiment the strands of the mixed duplex oligonucleotide are covalently bonded by a linker, such as a single stranded hexa, penta or tetranucleotide so that the first and second strands are segments of a single oligonucleotide chain having a single 3′ and a single 5′ end. The 3′ and 5′ ends can be protected by the addition of a “hairpin cap” whereby the 3′ and 5′ terminal nucleotides are Watson-Crick paired to adjacent nucleotides. A second hairpin cap can, additionally, be placed at the junction between the first and second strands distant from the 3′ and 5′ ends, so that the Watson-Crick pairing between the first and second strands is stabilized.

The first and second strands contain two regions that are homologous with two fragments of the target ACCase gene, i.e., have the same sequence as the target gene. A homologous region contains the nucleotides of an RNA segment and may contain one or more DNA-type nucleotides of connecting DNA segment and may also contain DNA-type nucleotides that are not within the intervening DNA segment. The two regions of homology are separated by, and each is adjacent to, a region having a sequence that differs from the sequence of the target gene, termed a “heterologous region.” The heterologous region can contain one, two or three mismatched nucleotides. The mismatched nucleotides can be contiguous or alternatively can be separated by one or two nucleotides that are homologous with the target gene. Alternatively, the heterologous region can also contain an insertion or one, two, three or of five or fewer nucleotides. Alternatively, the sequence of the mixed duplex oligonucleotide may differ from the sequence of the target gene only by the deletion of one, two, three, or five or fewer nucleotides from the mixed duplex oligonucleotide. The length and position of the heterologous region is, in this case, deemed to be the length of the deletion, even though no nucleotides of the mixed duplex oligonucleotide are within the heterologous region. The distance between the fragments of the target gene that are complementary to the two homologous regions is identically the length of the heterologous region when a substitution or substitutions is intended. When the heterologous region contains an insertion, the homologous regions are thereby separated in the mixed duplex oligonucleotide farther than their complementary homologous fragments are in the gene, and the converse is applicable when the heterologous region encodes a deletion.

The RNA segments of the mixed duplex oligonucleotides are each a part of a homologous region, i.e., a region that is identical in sequence to a fragment of the target gene, which segments together preferably contain at least 13 RNA-type nucleotides and preferably from 16 to 25 RNA-type nucleotides or yet more preferably 18-22 RNA-type nucleotides or most preferably 20 nucleotides. In one embodiment, RNA segments of the homology regions are separated by and adjacent to, i.e., “connected by” an intervening DNA segment. In one embodiment, each nucleotide of the heterologous region is a nucleotide of the intervening DNA segment. An intervening DNA segment that contains the heterologous region of a mixed duplex oligonucleotide is termed a “mutator segment.”

The change to be introduced into the target gene is encoded by the heterologous region. The change to be introduced into the ACCase gene may be a change in one or more bases of the target gene sequence that changes the native amino acid in that position to the desired amino acid.

In another embodiment of the present disclosure, the oligonucleobase is a single stranded oligodeoxynucleotide mutational vector or SSOMV, which is disclosed in International Patent Application PCT/US00/23457, which is incorporated herein by reference in its entirety. The sequence of the SSOMV is based on the same principles as the mutational vectors described in U.S. Pat. Nos. 5,756,325; 5,871,984; 5,760,012; 5,888,983; 5,795,972; 5,780,296; 5,945,339; 6,004,804; and 6,010,907 and in International Publication Nos. WO 98/49350; WO 99/07865; WO 99/58723; WO 99/58702; WO 99/40789; U.S. Pat. No. 6,870,075; and U.S. Published patent application No. 20030084473. The sequence of the SSOMV contains two regions that are homologous with the target sequence separated by a region that contains the desired genetic alteration termed the mutator region. The mutator region can have a sequence that is the same length as the sequence that separates the homologous regions in the target sequence but having a different sequence. Such a mutator region will cause a substitution.

The nucleotides of the SSOMV are deoxyribonucleotides that are linked by unmodified phosphodiester bonds except that the 3′ terminal and/or 5′ terminal internucleotide linkage or alternatively the two 3′ terminal and/or 5′ terminal internucleotide linkages can be a phosphorothioate or phosphoamidate. As used herein an internucleotide linkage is the linkage between nucleotides of the SSOMV and does not include the linkage between the 3′ end nucleotide or 5′ end nucleotide and a blocking substituent, see supra. In a specific embodiment the length of the SSOMV is between 21 and 55 deoxynucleotides and the lengths of the homology regions are, accordingly, a total length of at least 20 deoxynucleotides and at least two homology regions should each have lengths of at least 8 deoxynucleotides.

The SSOMV can be designed to be complementary to either the coding or the non-coding strand of the target gene. When the desired mutation is a substitution of a single base, it is preferred that both mutator nucleotides be a pyrimidine. To the extent that is consistent with achieving the desired functional result it is preferred that both the mutator nucleotide and the targeted nucleotide in the complementary strand be pyrimidines. Particularly preferred are SSOMV that encode transversion mutations, i.e., a C or T mutator nucleotide is mismatched, respectively, with a C or T nucleotide in the complementary strand.

In addition to the oligodeoxynucleotide the SSOMV can contain a 5′ blocking substituent that is attached to the 5′ terminal carbons through a linker. The chemistry of the linker is not critical other than its length, which should preferably be at least 6 atoms long and that the linker should be flexible. A variety of non-toxic substituents such as biotin, cholesterol or other steroids or a non-intercalating cationic fluorescent dye can be used. Particularly preferred as reagents to make SSOMV are the reagents sold as Cy3™ and Cy5™ by Glen Research, Sterling VA (acquired by Maravai LifeSciences), which are blocked phosphoroamidites that upon incorporation into an oligonucleotide yield 3,3,3′,3′-tetramethyl N,N′-isopropyl substituted indomonocarbocyanine and indodicarbocyanine dyes, respectively. Cy3 is the most preferred. When the indocarbocyanine is N-oxyalkyl substituted it can be conveniently linked to the 5′ terminal of the oligodeoxynucleotide through as a phosphodiester with a 5′ terminal phosphate. The chemistry of the dye linker between the dye and the oligodeoxynucleotide is not critical and is chosen for synthetic convenience. When the commercially available Cy3 phosphoramidite is used as directed the resulting 5′ modification consists of a blocking substituent and linker together which are a N-hydroxypropyl, N′-phosphatidylpropyl 3,3,3′,3′-tetramethyl indomonocarbocyanine.

In a preferred embodiment the indocarbocyanine dye is tetra substituted at the 3 and 3′ positions of the indole rings. Without limitation as to theory these substitutions prevent the dye from being an intercalating dye. The identity of the substituents at these positions is not critical. The SSOMV can in addition have a 3′ blocking substituent. Again, the chemistry of the 3′ blocking substituent is not critical.

In another embodiment the oligonucleotide may be a single-stranded oligodeoxynucleotide having a 3′ end nucleotide, a 5′ end nucleotide, having at least 25 deoxynucleotides and not more than 65 deoxynucleotides, and having a sequence comprising at least two regions each of at least 8 deoxynucleotides that are each, respectively, identical to at least two regions of the targeted chromosomal gene, which regions together are at least 24 nucleotides in length, and which regions are separated by at least one nucleotide in the sequence of the targeted chromosomal gene or in the sequence of the oligodeoxynucleotide or both such that the sequence of the oligodeoxynucleotide is not identical to the sequence of the targeted chromosomal gene. See U.S. Pat. No. 6,271,360 which is incorporated herein by reference.

The mutations herein described might also be obtained by mutagenesis (random, somatic or directed) and other DNA editing or nucleases using a repair template including, but not limited to, gene targeting using zinc finger nucleases, using Transcription Activator-Like Effector Nucleases (TALENs), using Clustered Regularly Interspaced Short Palindromic Repeats (CRISPRs). These nucleases can be plasmid (DNA) based, RNA and/or protein.

### Microcarriers and Microfibers

The use of metallic microcarriers (microspheres) for introducing large fragments of DNA into plant cells having cellulose cell walls by projectile penetration is well known to those skilled in the relevant art (henceforth biolistic delivery). U.S. Pat. Nos. 4,945,050; 5,100,792 and 5,204,253 describe general techniques for selecting microcarriers and devices for projecting them. U.S. Pat. Nos. 5,484,956 and 5,489,520 describe the preparation of fertile transgenic corn using microprojectile bombardment of corn callus tissue. The biolistic techniques are also used in transforming immature corn embryos.

Specific conditions for using microcarriers in the methods of the present disclosure are described in International Publication WO 99/07865. In an illustrative technique, ice cold microcarriers (60 mg/ml), mixed duplex oligonucleotide (60 mg/ml) 2.5 M CaCl₂ and 0.1 M spermidine are added in that order; the mixture is gently agitated, e.g., by vortexing, for 10 minutes and let stand at room temperature for 10 minutes, whereupon the microcarriers are diluted in 5 volumes of ethanol, centrifuged and resuspended in 100% ethanol. Good results can be obtained with a concentration in the adhering solution of 8-10 μg/μl microcarriers, 14-17 μg/ml mixed duplex oligonucleotide, 1.1-1.4 M CaCl₂ and 18-22 mM spermidine. Optimal results were observed under the conditions of 8 μg/μl microcarriers, 16.5 μg/ml mixed duplex oligonucleotide, 1.3 M CaCl₂ and 21 mM spermidine.

Oligonucleobases can also be introduced into plant cells for the practice of the present disclosure using microfibers to penetrate the cell wall and cell membrane. U.S. Pat. No. 5,302,523 to Coffee et al. describes the use of 30×0.5 μm and 10×0.3 μm silicon carbide fibers to facilitate transformation of suspension maize cultures of Black Mexican Sweet. Any mechanical technique that can be used to introduce DNA for transformation of a plant cell using microfibers can be used to deliver oligonucleobases for use in making the present ACCase mutants. The process disclosed by Coffee et al. in U.S. Pat. No. 5,302,523 can be employed with regenerable plant cell materials to introduce the present oligonucleobases to affect the mutation of the ACCase gene.

An illustrative technique for microfiber delivery of an oligonucleobase is as follows: Sterile microfibers (2 μg) are suspended in 150 μl of plant culture medium containing about 10 μg of a mixed duplex oligonucleotide. A suspension culture is allowed to settle, and equal volumes of packed cells and the sterile fiber/nucleotide suspension are vortexed for 10 minutes and plated. Selective media are applied immediately or with a delay of up to about 120 hours as is appropriate for the particular trait.

### Electroporation

In an alternative embodiment, the oligonucleobases can be delivered to the plant cell by electroporation of a protoplast derived from a plant part according to techniques that are well-known to one of ordinary skill in the art. See, e.g., Gallois et al., 1996, in Methods in Molecular Biology 55:89-107, Humana Press, Totowa, N.J.; Kipp et al., 1999, in Methods in Molecular Biology 133:213-221, Humana Press, Totowa, N.J.

Oligonucleobases can also be introduced into microspores by electroporation. Upon release of the tetrad, the microspore is uninucleate and thin-walled. It begins to enlarge and develops a germpore before the exine forms. A microspore at this stage is potentially more amenable to transformation with exogenous DNA than other plant cells. In addition, microspore development can be altered in vitro to produce either haploid embryos or embryogenic callus that can be regenerated into plants (Coumans et al., Plant Cell Rep. 7:618-621, 1989; Datta et al., Plant Sci. 67:83-88, 1990; Maheshwari et al., Am. J Bot. 69:865-879, 1982; Schaeffer, Adv. In Cell Culture 7:161-182, 1989; Swanson et al., Plant Cell Rep. 6:94-97, 1987). Thus, transformed microspores can be regenerated directly into haploid plants or dihaploid fertile plants upon chromosome doubling by standard methods. See also co-pending application U.S. Ser. No. 09/680,858 entitled Compositions and Methods for Plant Genetic Modification which is incorporated herein by reference.

Microspore electroporation can be practiced with any plant species for which microspore culture is possible, including but not limited to plants in the families Graminae, Leguminoceae, Cruciferaceae, Solanaceae, Cucurbitaceae, Rosaccae, Poaceae, Lilaceae, Rutaceae, Vitaceae, including such species as corn (Zea mays), wheat (Triticum aestivum), rice (Oryza sativa), oats (Avena sativa), barley (Hordeum vulgare), canola (Brassica napus, Brassica rapa, Brassica oleracea, and Brassica juncea), cotton (Gossypium hirsuitum L.), various legume species (e.g., soybean (Glycine max), pea (Pisum sativum), etc.), grapes (Vitis vinifera), and a host of other important crop plants. Microspore embryogenesis, both from anther and microspore culture, has been described in more than 170 species, belonging to 68 genera and 28 families of dicotyledons and monocotyledons (Raghavan, Embryogenesis in Angiosperms: A Developmental and Experimental Study, Cambridge University Press, Cambridge, England, 1986; Rhagavan, Cell Differentiation 21:213-226, 1987; Raemakers et al., Euphytica 81:93-107, 1995). For a detailed discussion of microspore isolation, culture, and regeneration of double haploid plants from microspore-derived embryos (MDE) in Brassica napus L., see Nehlin, The Use of Rapeseed (Brassica napus L.) Microspores as a Tool for Biotechnological Applications, doctoral thesis, Swedish University of Agricultural Sciences, Uppsala, Sweden, 1999; also Nehlin et al., Plant Sci. 111:219-227, 1995, and Nehlin et al., Plant Sci. 111:219-227, 1995). Chromosome doubling from microspore or anther culture is a well-established technique for production of double-haploid homozygous plant lines in several crops (Heberle-Bors et al., In vitro pollen cultures: Progress and perspectives. In: Pollen Biotechnology. Gene expression and allergen characterization, vol. 85-109, ed. Mohapatra, S. S., and Knox, R. B., Chapman and Hall, New York, 1996).

Microspore electroporation methods are described in Jardinaud et al., Plant Sci. 93:177-184, 1993, and Fennell and Hauptman, Plant Cell Reports 11:567-570, 1992. Methods for electroporation of MDON into plant protoplasts can also be adapted for use in microspore electroporation.

### Whiskers Technique

In yet another alternative embodiment, the oligonucleobase can be delivered to the plant cell by whiskers or microinjection of the plant cell. The so-called whiskers technique is performed essentially as described in Frame et al., 1994, Plant J. 6:941-948. The oligonucleobase is added to the whiskers and used to transform the plant cells. The oligonucleobase may be co-incubated with plasmids comprising sequences encoding proteins capable of forming recombinase complexes in plant cells such that recombination is catalyzed between the oligonucleotide and the target sequence in the ACCase gene.

### Other Delivery Methods

In an alternative embodiment, nucleic acids are embedded in microbeads composed of calcium alginate and taken up by plant protoplasts in the presence of the membrane-modifying agent polyethylene glycol (see, e.g., Sone et al., 2002; Liu et al., 2004).

In an alternative embodiment, nucleic acids frozen in water and introduced into plant cells by bombardment in the form of microparticles (see, e.g., Gilmore, 1991, U.S. Pat. No. 5,219,746; Brinegar et al.).

In an alternative embodiment, nucleic acids attached to nanoparticles are introduced into intact plant cells by incubation of the cells in a suspension containing the nanoparticle (see, e.g., Pasupathy et al., 2008) or by delivering them into intact cells through particle bombardment or into protoplasts by co-incubation (see, e.g., Torney et al., 2007).

In an alternative embodiment, nucleic acids complexed with penetrating peptides are delivered into cells by co-incubation (see, e.g., Chugh et al., 2008, WO 2008148223 A1; Eudes and Chugh).

In an alternative embodiment, nucleic acids are introduced into intact cells through electroporation (see, e.g., He et al., 1998, U.S. 2003/0115641 A1, Dobres et al.).

In an alternative embodiment, nucleic acids are delivered into cells of dry embryos by soaking them in a solution with nucleic acids (by soaking dry embryos in (see, e.g., Töpfer et al., 1989; Senaratna et al., 1991).

### Targeted Gene Modification

Targeted genetic modification mediated by oligonucleotides is a valuable technique for use in the specific alteration of short stretches of DNA to create or make deletions, short insertions, and point mutations and may be used in conjunction with the disclosures herein, for example to cause one or more of the ACCase mutations contemplated herein. These methods may in some embodiments involve DNA pairing/annealing, followed by a DNA repair event. First, the nucleic acid anneals with its complementary strand in the double-stranded DNA in a process mediated by cellular protein factors. This annealing creates a centrally located mismatched base pair (in the case of a point mutation), resulting in a structural perturbation that most likely stimulates the endogenous protein machinery to initiate the second step in the repair process: site-specific modification of the chromosomal sequence and/or that in organelles (e.g., mitochondria and chloroplasts). This newly introduced mismatch induces the DNA repair machinery to perform a second repair event, leading to the final revision of the target site. The present methods and compositions in various aspects and embodiments disclosed herein, may improve the methods by providing novel approaches which increase the availability of DNA repair components, thus increasing the efficiency and reproducibility of gene repair-mediated modifications to targeted nucleic acids.

Efficient methods for site-directed genomic modifications are desirable for research, clinical gene therapy, industrial microbiology and agriculture. One approach utilizes triplex-forming oligonucleotides (TFO) which bind as third strands to duplex DNA in a sequence-specific manner, to mediate directed mutagenesis. Such TFO can act either by delivering a tethered mutagen, such as psoralen or chlorambucil (Havre et al., Proc Nat'l Acad Sci, U.S.A. 90:7879-7883, 1993; Havre et al., J Virol 67:7323-7331, 1993; Wang et al., Mol Cell Biol 15:1759-1768, 1995; Takasugi et al., Proc Nat'l Acad Sci, U.S.A. 88:5602-5606, 1991; Belousov et al., Nucleic Acids Res 25:3440-3444, 1997), or by binding with sufficient affinity to provoke error-prone repair (Wang et al., Science 271:802-805, 1996).

Another strategy for genomic modification that may be used in conjunction with the compositions and methods herein involves the induction of homologous recombination between an exogenous DNA fragment and the targeted gene. This approach has been used successfully to target and disrupt selected genes in mammalian cells and has enabled the production of transgenic mice carrying specific gene knockouts (Capeechi et al., Science 244:1288-1292, 1989; Wagner, U.S. Pat. No. 4,873,191). This approach involves the transfer of selectable markers to allow isolation of the desired recombinants. Without selection, the ratio of homologous to non-homologous integration of transfected DNA in typical gene transfer experiments is low, usually in the range of 1:1000 or less (Sedivy et al., Gene Targeting, W. H. Freeman and Co., New York, 1992). This low efficiency of homologous integration limits the utility of gene transfer for experimental use or gene therapy. The frequency of targeted mutation can be enhanced by damage to the target site from UV irradiation and selected carcinogens (Wang et al., Mol Cell Biol 8:196-202, 1988) as well as by site-specific endonucleases (Sedivy et al, Gene Targeting, W. H. Freeman and Co., New York, 1992; Rouet et al., Proc Nat'l Acad Sci, U.S.A. 91:6064-6068, 1994; Segal et al., Proc Nat'l Acad Sci, U.S.A. 92:806-810, 1995). In addition, DNA damage induced by triplex-directed psoralen photoadducts can stimulate recombination within and between extrachromosomal vectors (Segal et al., Proc Nat'l Acad Sci, U.S.A. 92:806-810, 1995; Faruqi et al., Mol Cell Biol 16:6820-6828, 1996; Glazer, U.S. Pat. No. 5,962,426).

Linear donor fragments are more efficacious for targeted mutation than their circular counterparts (Folger et al., Mol Cell Biol 2:1372-1387, 1982). Recombination can in certain embodiments also be influenced by the length of uninterrupted homology between both the donor and target sites, with short fragments often appearing to be ineffective substrates (Rubnitz et al., Mol Cell Biol 4:2253-2258, 1984). Nonetheless, the use of short fragments of DNA or DNA/RNA hybrids for gene correction is the focus of various strategies. (Kunzelmann et al., Gene Ther 3:859-867, 1996).

“Nucleic acid sequence,” “nucleotide sequence” and “polynucleotide sequence” as used herein refer to an oligonucleotide or polynucleotide, and fragments or portions thereof, and to DNA or RNA of genomic or synthetic origin which may be single- or double-stranded and represent the sense or antisense strand.

As used herein, the terms “oligonucleotide” and “oligomer” refer to a polymer of nucleobases. In some embodiments an “oligonucleotide” or “oligomer” may be of at least about 8 nucleobases or may have as many as about 1,500 nucleobases or more. In certain embodiments, an “oligonucleotide” or “oligomer” may be any length as contemplated herein.

The terms “DNA-modifying molecule” and “DNA-modifying reagent” as used herein refer to a molecule which is capable of recognizing and specifically binding to a nucleic acid sequence in the genome of a cell, and which is capable of modifying a target nucleotide sequence within the genome, wherein the recognition and specific binding of the DNA-modifying molecule to the nucleic acid sequence is protein-independent. The term “protein-independent” as used herein in connection with a DNA-modifying molecule means that the DNA-modifying molecule does not require the presence and/or activity of a protein and/or enzyme for the recognition of, and/or specific binding to, a nucleic acid sequence. DNA-modifying molecules are exemplified, but not limited to triplex forming oligonucleotides, peptide nucleic acids, polyamides, and oligonucleotides which are intended to promote gene conversion. The DNA-modifying molecules of the present disclosure are in certain embodiments distinguished from the prior art's nucleic acid sequences which are used for homologous recombination (Wong & Capecchi, Molec. Cell. Biol. 7:2294-2295, 1987) in that the prior art's nucleic acid sequences which are used for homologous recombination are protein-dependent. The term “protein-dependent” as used herein in connection with a molecule means that the molecule requires the presence and/or activity of a protein and/or enzyme for the recognition of, and/or specific binding of the molecule to, a nucleic acid sequence. Methods for determining whether a DNA-modifying molecule requires the presence and/or activity of a protein and/or enzyme for the recognition of, and/or specific binding to, a nucleic acid sequence are within the skill in the art (see, e.g., Dennis et al. Nucl. Acids Res. 27:4734-4742, 1999). For example, the DNA-modifying molecule may be incubated in vitro with the nucleic acid sequence in the absence of any proteins and/or enzymes. The detection of specific binding between the DNA-modifying molecule and the nucleic acid sequence demonstrates that the DNA-modifying molecule is protein-independent. On the other hand, the absence of specific binding between the DNA-modifying molecule and the nucleic acid sequence demonstrates that the DNA-modifying molecule is protein-dependent and/or requires additional factors.

“Triplex forming oligonucleotide” (TFO) is defined as a sequence of DNA or RNA that is capable of binding in the major grove of a duplex DNA or RNA helix to form a triple helix. Although the TFO is not limited to any particular length, a preferred length of the TFO is 250 nucleotides or less, 200 nucleotides or less, or 100 nucleotides or less, or from 5 to 50 nucleotides, or from 10 to 25 nucleotides, or from 15 to 25 nucleotides. Although a degree of sequence specificity between the TFO and the duplex DNA is necessary for formation of the triple helix, no particular degree of specificity is required, as long as the triple helix is capable of forming. Likewise, no specific degree of avidity or affinity between the TFO and the duplex helix is required as long as the triple helix is capable of forming. While not intending to limit the length of the nucleotide sequence to which the TFO specifically binds in one embodiment, the nucleotide sequence to which the TFO specifically binds is from 1 to 100, in some embodiments from 5 to 50, yet other embodiments from 10 to 25, and in other embodiments from 15 to 25, nucleotides. Additionally, “triple helix” is defined as a double-helical nucleic acid with an oligonucleotide bound to a target sequence within the double-helical nucleic acid. The “double-helical” nucleic acid can be any double-stranded nucleic acid including double-stranded DNA, double-stranded RNA and mixed duplexes of DNA and RNA. The double-stranded nucleic acid is not limited to any particular length. However, in preferred embodiments it has a length of greater than 500 bp, in some embodiments greater than 1 kb and in some embodiments greater than about 5 kb. In many applications the double-helical nucleic acid is cellular, genomic nucleic acid. The triplex forming oligonucleotide may bind to the target sequence in a parallel or anti-parallel manner.

“Peptide Nucleic Acids,” “polyamides” or “PNA” are nucleic acids wherein the phosphate backbone is replaced with an N-aminoethylglycine-based polyamide structure. PNAs have a higher affinity for complementary nucleic acids than their natural counter parts following the Watson-Crick base-pairing rules. PNAs can form highly stable triple helix structures with DNA of the following stoichiometry: (PNA) 2.DNA. Although the peptide nucleic acids and polyamides are not limited to any particular length, a preferred length of the peptide nucleic acids and polyamides is 200 nucleotides or less, in some embodiments 100 nucleotides or less, and in some embodiments from 5 to 50 nucleotides long. While not intending to limit the length of the nucleotide sequence to which the peptide nucleic acid and polyamide specifically binds, in one embodiment, the nucleotide sequence to which the peptide nucleic acid and polyamide specifically bind is from 1 to 100, in some embodiments from 5 to 50, yet other embodiments from 5 to 25, and other embodiments from 5 to 20, nucleotides.

The term “capable of modifying DNA” or “DNA modifying means” refers to procedures, as well as endogenous or exogenous agents or reagents that have the ability to induce, or can aid in the induction of, changes to the nucleotide sequence of a targeted segment of DNA. Such changes may be made by the deletion, addition or substitution of one or more bases on the targeted DNA segment. It is not necessary that the DNA sequence changes confer functional changes to any gene encoded by the targeted sequence. Furthermore, it is not necessary that changes to the DNA be made to any particular portion or percentage of the cells.

The term “nucleotide sequence of interest” refers to any nucleotide sequence, the manipulation of which may be deemed desirable for any reason, by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences that do not encode an mRNA or protein product (e.g., promoter sequence, enhancer sequence, polyadenylation sequence, termination sequence, regulatory RNAs such as miRNA, etc.).

“Amino acid sequence,” “polypeptide sequence,” “peptide sequence” and “peptide” are used interchangeably herein to refer to a sequence of amino acids.

“Target sequence,” as used herein, refers to a double-helical nucleic acid comprising a sequence that is the subject of interest. In some embodiments a target sequence may be greater than 8 nucleotides in length and in some embodiments less than 1,500 nucleotides in length. In some embodiments, the target sequence is between 8 to 30 bases. In some embodiments the target sequence may be between about 75 and 250 bases in length. In certain embodiments the target sequence may be a length complimentary to the length of an oligonucleotide as contemplated herein. The target sequence, in general, is defined by the nucleotide sequence on one of the strands on the double-helical nucleic acid.

As used herein, a “purine-rich sequence” or “polypurine sequence” when made in reference to a nucleotide sequence on one of the strands of a double-helical nucleic acid sequence is defined as a contiguous sequence of nucleotides wherein greater than 50% of the nucleotides of the target sequence contain a purine base. However, it is preferred that the purine-rich target sequence contain greater than 60% purine nucleotides, in some embodiments greater than 75% purine nucleotides, in other embodiments greater than 90% purine nucleotides and yet other embodiments 100% purine nucleotides.

As used herein, a “pyrimidine-rich sequence” or “polypyrimidine sequence” when made in reference to a nucleotide sequence on one of the strands of a double-helical nucleic acid sequence is defined as a contiguous sequence of nucleotides wherein greater that 50% of the nucleotides of the target sequence contain a pyrimidine base. However, it is preferred that the pyrimidine-rich target sequence contain greater than 60% pyrimidine nucleotides and, in some embodiments, greater than 75% pyrimidine nucleotides. In some embodiments, the sequence contains greater than 90% pyrimidine nucleotides and, in other embodiments, is 100% pyrimidine nucleotides.

A “variant” of a first nucleotide sequence is defined as a nucleotide sequence which differs from the first nucleotide sequence (e.g., by having one or more deletions, insertions, or substitutions that may be detected using hybridization assays or using DNA sequencing). Included within this definition is the detection of alterations or modifications to the genomic sequence of the first nucleotide sequence. For example, hybridization assays may be used to detect (1) alterations in the pattern of restriction enzyme fragments capable of hybridizing to the first nucleotide sequence when comprised in a genome (i.e., RFLP analysis), (2) the inability of a selected portion of the first nucleotide sequence to hybridize to a sample of genomic DNA which contains the first nucleotide sequence (e.g., using allele-specific oligonucleotide probes), (3) improper or unexpected hybridization, such as hybridization to a locus other than the normal chromosomal locus for the first nucleotide sequence (e.g., using fluorescent in situ hybridization (FISH) to metaphase chromosomes spreads, etc.). One example of a variant is a mutated wild-type sequence.

The terms “nucleic acid” and “unmodified nucleic acid” as used herein refer to any one of the known four deoxyribonucleic acid bases (i.e., guanine, adenine, cytosine, and thymine). The term “modified nucleic acid” refers to a nucleic acid whose structure is altered relative to the structure of the unmodified nucleic acid. Illustrative of such modifications would be replacement covalent modifications of the bases, such as alkylation of amino and ring nitrogens as well as saturation of double bonds.

As used herein, the terms “mutation” and “modification” and grammatical equivalents thereof when used in reference to a nucleic acid sequence are used interchangeably to refer to a deletion, insertion, substitution, strand break, and/or introduction of an adduct. A “deletion” is defined as a change in a nucleic acid sequence in which one or more nucleotides is absent. An “insertion” or “addition” is that change in a nucleic acid sequence which has resulted in the addition of one or more nucleotides. A “substitution” results from the replacement of one or more nucleotides by a molecule which is a different molecule from the replaced one or more nucleotides. For example, a nucleic acid may be replaced by a different nucleic acid as exemplified by replacement of a thymine by a cytosine, adenine, guanine, or uridine. Pyrimidine to pyrimidine (e.g., C to T or T to C nucleotide substitutions) or purine to purine (e.g., G to A or A to G nucleotide substitutions) are termed transitions, whereas pyrimidine to purine or purine to pyrimidine (e.g., G to T or G to C or A to T or A to C) are termed transversions. Alternatively, a nucleic acid may be replaced by a modified nucleic acid as exemplified by replacement of a thymine-by-thymine glycol. Mutations may result in a mismatch. The term “mismatch” refers to a non-covalent interaction between two nucleic acids, each nucleic acid residing on a different polynucleic acid sequence, which does not follow the base-pairing rules. For example, for the partially complementary sequences 5′-AGT-3′ and 5′-AAT-3′, a G-A mismatch (a transition) is present. The terms “introduction of an adduct” or “adduct formation” refer to the covalent or non-covalent linkage of a molecule to one or more nucleotides in a DNA sequence such that the linkage results in a reduction (in some embodiments from 10% to 100%, in other embodiments from 50% to 100%, and in some embodiments from 75% to 100%) in the level of DNA replication and/or transcription.

The term “DNA cutter” refers to a moiety that effects a strand break. Non-limited examples include meganucleases, TALEs/TALENs, antibiotics, zinc fingers and CRISPRs, which include but are not limited to Cas9, Cpf1 and their corresponding homologues, orthologues and/or paralogues, a base editor, or CRISPR/Cas systems.

The term “strand break” when made in reference to a double stranded nucleic acid sequence includes a single-strand break and/or a double-strand break. A single-strand break (a nick) refers to an interruption in one of the two strands of the double stranded nucleic acid sequence. This is in contrast to a double-strand break which refers to an interruption in both strands of the double stranded nucleic acid sequence, which may result in blunt or staggered ends. Strand breaks may be introduced into a double stranded nucleic acid sequence either directly (e.g., by ionizing radiation or treatment with certain chemicals) or indirectly (e.g., by enzymatic incision at a nucleic acid base). In certain embodiments, a DNA cutter may have selectivity for certain specific sequences, such as in the case of a CRISPR, which includes but is not limited to Cas9, Cpf1 and their corresponding homologues, orthologues and/or paralogues, a base editor, a zinc finger, a meganuclease, a TALEN as described herein.

The terms “mutant cell” and “modified cell” refer to a cell which contains at least one modification in the cell's genomic sequence.

The term “portion” when used in reference to a nucleotide sequence refers to fragments of that nucleotide sequence. The fragments may range in size from 5 nucleotide residues to the entire nucleotide sequence minus one nucleic acid residue.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects that transcription proceeds in a 5′ to 3′ direction along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

The term “recombinant DNA molecule” as used herein refers to a DNA molecule which is comprised of segments of DNA joined together by means of molecular biological techniques.

The term “recombinant protein” or “recombinant polypeptide” as used herein refers to a protein molecule which is expressed using a recombinant DNA molecule.

As used herein, the terms “vector” and “vehicle” are used interchangeably in reference to nucleic acid molecules that transfer one or more DNA segment from one cell to another.

The terms “in operable combination,” “in operable order” and “operably linked” as used herein refer to the linkage of nucleic acid sequences in such a manner that a nucleic acid molecule capable of directing the transcription of a given gene and/or the synthesis of a desired protein molecule is produced. The terms also refer to the linkage of amino acid sequences in such a manner so that a functional protein is produced.

The term “transfection” as used herein refers to the introduction of foreign DNA into cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofectin, protoplast fusion, retroviral infection, biolistics (i.e., particle bombardment) and the like.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-CAGT-3′,” is complementary to the sequence “5′-ACTG-3′.” Complementarity can be “partial” or “total”. “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands may have significant effects on the efficiency and strength of hybridization between nucleic acid strands. This may be of particular importance in amplification reactions, as well as detection methods which depend upon binding between nucleic acids. For the sake of convenience, the terms “polynucleotides” and “oligonucleotides” include molecules which include nucleosides.

The terms “homology” and “homologous” as used herein in reference to nucleotide sequences refer to a degree of complementarity with other nucleotide sequences. There may be partial homology or complete homology (i.e., identity). When used in reference to a double-stranded nucleic acid sequence such as a cDNA or genomic clone, the term “substantially homologous” refers to any nucleic acid sequence (e.g., probe) which can hybridize to either or both strands of the double-stranded nucleic acid sequence under conditions of low stringency as described above. A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

Low stringency conditions comprise conditions equivalent to binding or hybridization at 68° C. in a solution consisting of 5×SSPE (43.8 g/l NaCl, 6.9 g/l NaH₂PO₄·H₂O and 1.85 g/l EDTA, pH adjusted to 7.4 with NaOH), 0.1% SDS, 5×Denhardt's reagent (50×Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 2.0×SSPE, 0.1% SDS at room temperature when a probe of about 100 to about 1000 nucleotides in length is employed.

In addition, conditions which promote hybridization under conditions of high stringency (e.g., increasing the temperature of the hybridization and/or wash steps, the use of formamide in the hybridization solution, etc.) are well known in the art. High stringency conditions, when used in reference to nucleic acid hybridization, comprise conditions equivalent to binding or hybridization at 68° C. in a solution consisting of 5×SSPE, 1% SDS, 5×Denhardt's reagent and 100 μg/ml denatured salmon sperm DNA followed by washing in a solution comprising 0.1×SSPE and 0.1% SDS at 68° C. when a probe of about 100 to about 1000 nucleotides in length is employed.

It is well known in the art that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol), as well as components of the hybridization solution may be varied to generate conditions of low stringency hybridization different from, but equivalent to, the above listed conditions.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 50% to 70% homology to the first nucleic acid sequence, then another hybridization condition is said to be equivalent to the hybridization condition of interest if this other hybridization condition also results in hybridization of the first nucleic acid sequence with the other nucleic acid sequences that have from 50% to 70% homology to the first nucleic acid sequence.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein the term “hybridization complex” refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41 (% G+C), when a nucleic acid is in aqueous solution at 1 M NaCl (see e.g., Anderson and Young, Quantitative Filter Hybridization, in Nucleic Acid Hybridization, 1985). Other references include more sophisticated computations which take structural as well as sequence characteristics into account for the calculation of Tm.

As used herein the term “stringency” is used in reference to the conditions of temperature, ionic strength, and the presence of other compounds such as organic solvents, under which nucleic acid hybridizations are conducted. “Stringency” typically occurs in a range from about Tm−5° C. (5° C. below the melting temperature of the probe) to about 20° C. to 25° C. below Tm. As will be understood by those of skill in the art, a stringent hybridization can be used to identify or detect identical polynucleotide sequences or to identify or detect similar or related polynucleotide sequences.

The terms “specific binding,” “binding specificity,” and grammatical equivalents thereof when made in reference to the binding of a first nucleotide sequence to a second nucleotide sequence, refer to the preferential interaction between the first nucleotide sequence with the second nucleotide sequence as compared to the interaction between the second nucleotide sequence with a third nucleotide sequence. Specific binding is a relative term that does not require absolute specificity of binding; in other words, the term “specific binding” does not require that the second nucleotide sequence interact with the first nucleotide sequence in the absence of an interaction between the second nucleotide sequence and the third nucleotide sequence. Rather, it is sufficient that the level of interaction between the first nucleotide sequence and the second nucleotide sequence is greater than the level of interaction between the second nucleotide sequence with the third nucleotide sequence. “Specific binding” of a first nucleotide sequence with a second nucleotide sequence also means that the interaction between the first nucleotide sequence and the second nucleotide sequence is dependent upon the presence of a particular structure on or within the first nucleotide sequence; in other words the second nucleotide sequence is recognizing and binding to a specific structure on or within the first nucleotide sequence rather than to nucleic acids or to nucleotide sequences in general. For example, if a second nucleotide sequence is specific for structure “A” that is on or within a first nucleotide sequence, the presence of a third nucleic acid sequence containing structure A will reduce the amount of the second nucleotide sequence which is bound to the first nucleotide sequence.

As used herein, the term “amplifiable nucleic acid” is used in reference to nucleic acids which may be amplified by any amplification method. It is contemplated that “amplifiable nucleic acid” will usually comprise “sample template.”

The terms “heterologous nucleic acid sequence” or “heterologous DNA” are used interchangeably to refer to a nucleotide sequence which is ligated to a nucleic acid sequence to which it is not ligated in nature, or to which it is ligated at a different location in nature. Heterologous DNA is not endogenous to the cell into which it is introduced but has been obtained from another cell. Generally, although not necessarily, such heterologous DNA encodes RNA and proteins that are not normally produced by the cell into which it is expressed. Examples of heterologous DNA include reporter genes, transcriptional and translational regulatory sequences, selectable marker proteins (e.g., proteins which confer drug resistance), etc.

“Amplification” is defined as the production of additional copies of a nucleic acid sequence and is generally carried out using polymerase chain reaction technologies well known in the art (Dieffenbach and Dveksler (1995) PCR Primer, a Laboratory Manual, Cold Spring Harbor Press, Plainview, N.Y.). As used herein, the term “polymerase chain reaction” (“PCR”) refers to the method of K. B. Mullis U.S. Pat. Nos. 4,683,195, and 4,683,202, hereby incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

One such preferred method, particularly for commercial applications, is based on the widely used TaqMan® real-time PCR technology and combines Allele-Specific PCR with a Blocking reagent (ASB-PCR) to suppress amplification of the wild-type allele. ASB-PCR can be used for detection of germ line or somatic mutations in either DNA or RNA extracted from any type of tissue, including formalin-fixed paraffin-embedded tumor specimens. A set of reagent design rules are developed enabling sensitive and selective detection of single point substitutions, insertions, or deletions against a background of wild-type allele in thousand-fold or greater excess. (Morlan, Baker, Sinicropi, Mutation Detection by Real-Time PCR: A Simple, Robust and Highly Selective Method. PLOS ONE 4 (2): e4584, 2009)

The terms “reverse transcription polymerase chain reaction” and “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using a single primer (e.g., an oligo-dT primer) prior to PCR amplification of the desired segment of the transcribed DNA using two primers.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and of an inducing agent such as DNA polymerase and at a suitable temperature and pH). In some embodiments, the primer is single stranded for maximum efficiency in amplification but may alternatively be double stranded. If double stranded, the primer is first treated to separate its strands before being used to prepare extension products. In some embodiments, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present disclosure will be labeled with any “reporter molecule,” so that it is detectable in any detection system, including, but not limited to enzyme (e.g., ELISA, as well as enzyme-based histochemical assays), fluorescent, radioactive, and luminescent systems. It is not intended that the present disclosure be limited to any particular detection system or label.

As used herein, the terms “restriction endonucleases” and “restriction enzymes” refer to bacterial enzymes, each of which cut or nick double- or single-stranded DNA at or near a specific nucleotide sequence, for example, an endonuclease domain of a type IIS restriction endonuclease (e.g., FokI can be used, as taught by Kim et al., 1996, Proc. Nat'l. Acad. Sci. U.S.A., 6:1 156-60).

As used herein, the term “an oligonucleotide having a nucleotide sequence encoding a gene” means a nucleic acid sequence comprising the coding region of a gene, i.e., the nucleic acid sequence which encodes a gene product. The coding region may be present in either a cDNA, genomic DNA or RNA form. When present in a DNA form, the oligonucleotide may be single-stranded (i.e., the sense strand) or double-stranded. Additionally, “an oligonucleotide having a nucleotide sequence encoding a gene” may include suitable control elements such as enhancers, promoters, splice junctions, polyadenylation signals, etc. if needed to permit proper initiation of transcription and/or correct processing of the primary RNA transcript. Further still, the coding region of the present disclosure may contain endogenous enhancers, splice junctions, intervening sequences, polyadenylation signals, etc.

Transcriptional control signals in eukaryotes comprise “enhancer” elements. Enhancers consist of short arrays of DNA sequences that interact specifically with cellular proteins involved in transcription (Maniatis, T. et al., Science 236:1237, 1987). Enhancer elements have been isolated from a variety of eukaryotic sources including genes in plant, yeast, insect and mammalian cells and viruses. The selection of a particular enhancer depends on what cell type is to be used to express the protein of interest.

The presence of “splicing signals” on an expression vector often results in higher levels of expression of the recombinant transcript. Splicing signals mediate the removal of introns from the primary RNA transcript and consist of a splice donor and acceptor site (Sambrook et al., Molecular Cloning: A Laboratory Manual, 2nd ed., Cold Spring Harbor Laboratory Press, New York, pp. 16.7-16.8, 1989). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is isolated from one gene and placed 3′ of another gene.

The term “promoter,” “promoter element” or “promoter sequence” as used herein, refers to a DNA sequence which when placed at the 5′ end of (i.e., precedes) an oligonucleotide sequence is capable of controlling the transcription of the oligonucleotide sequence into mRNA. A promoter is typically located 5′ (i.e., upstream) of an oligonucleotide sequence whose transcription into mRNA it controls and provides a site for specific binding by RNA polymerase and for initiation of transcription.

The term “promoter activity” when made in reference to a nucleic acid sequence refers to the ability of the nucleic acid sequence to initiate transcription of an oligonucleotide sequence into mRNA.

The term “tissue specific” as it applies to a promoter refers to a promoter that is capable of directing selective expression of an oligonucleotide sequence to a specific type of tissue in the relative absence of expression of the same oligonucleotide in a different type of tissue. Tissue specificity of a promoter may be evaluated by, for example, operably linking a reporter gene to the promoter sequence to generate a reporter construct, introducing the reporter construct into the genome of a plant or an animal such that the reporter construct is integrated into every tissue of the resulting transgenic animal, and detecting the expression of the reporter gene (e.g., detecting mRNA, protein, or the activity of a protein encoded by the reporter gene) in different tissues of the transgenic plant or animal. Selectivity need not be absolute. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter which is capable of directing selective expression of an oligonucleotide sequence in a specific type of cell in the relative absence of expression of the same oligonucleotide sequence in a different type of cell within the same tissue. The term “cell type specific” when applied to a promoter also means a promoter capable of promoting selective expression of an oligonucleotide in a region within a single tissue. Again, selectivity need not be absolute. Cell type specificity of a promoter may be assessed using methods well known in the art, e.g., immunohistochemical staining as described herein. Briefly, tissue sections are embedded in paraffin, and paraffin sections are reacted with a primary antibody which is specific for the polypeptide product encoded by the oligonucleotide sequence whose expression is controlled by the promoter. As an alternative to paraffin sectioning, samples may be cryosectioned. For example, sections may be frozen prior to and during sectioning thus avoiding potential interference by residual paraffin. A labeled (e.g., peroxidase conjugated) secondary antibody which is specific for the primary antibody is allowed to bind to the sectioned tissue and specific binding detected (e.g., with avidin/biotin) by microscopy.

The terms “selective expression,” “selectively express” and grammatical equivalents thereof refer to a comparison of relative levels of expression in two or more regions of interest. For example, “selective expression” when used in connection with tissues refers to a substantially greater level of expression of a gene of interest in a particular tissue, or to a substantially greater number of cells which express the gene within that tissue, as compared, respectively, to the level of expression of, and the number of cells expressing, the same gene in another tissue (i.e., selectivity need not be absolute). Selective expression does not require, although it may include expression of a gene of interest in a particular tissue and a total absence of expression of the same gene in another tissue. Similarly, “selective expression” as used herein in reference to cell types refers to a substantially greater level of expression of, or a substantially greater number of cells which express, a gene of interest in a particular cell type, when compared, respectively, to the expression levels of the gene and to the number of cells expressing the gene in another cell type.

The term “contiguous” when used in reference to two or more nucleotide sequences means the nucleotide sequences are ligated in tandem either in the absence of intervening sequences, or in the presence of intervening sequences which do not comprise one or more control elements.

As used herein, the terms “nucleic acid molecule encoding,” “nucleotide encoding,” “DNA sequence encoding” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “isolated” when used in relation to a nucleic acid, as in “an isolated oligonucleotide” refers to a nucleic acid sequence that is separated from at least one contaminant nucleic acid with which it is ordinarily associated in its natural source. Isolated nucleic acid is nucleic acid present in a form or setting that is different from that in which it is found in nature. In contrast, non-isolated nucleic acids are nucleic acids such as DNA and RNA which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, isolated nucleic acid encoding a polypeptide of interest includes, by way of example, such nucleic acid in cells ordinarily expressing the polypeptide of interest where the nucleic acid is in a chromosomal or extrachromosomal location different from that of natural cells or is otherwise flanked by a different nucleic acid sequence than that found in nature. The isolated nucleic acid or oligonucleotide may be present in single-stranded or double-stranded form. Isolated nucleic acid can be readily identified (if desired) by a variety of techniques (e.g., hybridization, dot blotting, etc.). When an isolated nucleic acid or oligonucleotide is to be utilized to express a protein, the oligonucleotide will contain at a minimum the sense or coding strand (i.e., the oligonucleotide may be single-stranded). Alternatively, it may contain both the sense and anti-sense strands (i.e., the oligonucleotide may be double-stranded).

As used herein, the term “purified” or “to purify” refers to the removal of one or more (undesired) components from a sample. For example, where recombinant polypeptides are expressed in bacterial host cells, the polypeptides are purified by the removal of host cell proteins thereby increasing the percent of recombinant polypeptides in the sample.

As used herein, the term “substantially purified” refers to molecules, either nucleic or amino acid sequences, that are removed from their natural environment, isolated or separated, and are at least 60% free, in some embodiments 75% free and other embodiments 90% free from other components with which they are naturally associated. An “isolated polynucleotide” is, therefore, a substantially purified polynucleotide.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side generally by the nucleotide triplet “ATG” which encodes the initiator methionine and on the 3′ side by one of the three triplets which specify stop codons (i.e., TAA, TAG, TGA).

By “coding sequence” is meant a sequence of a nucleic acid or its complement, or a part thereof, that can be transcribed and/or translated to produce the mRNA for and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the encoding sequence can be deduced therefrom.

By “non-coding sequence” is meant a sequence of a nucleic acid or its complement, or a part thereof that is not transcribed into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, etc.

As used herein, the term “structural gene” or “structural nucleotide sequence” refers to a DNA sequence coding for RNA or a protein which does not control the expression of other genes. In contrast, a “regulatory gene” or “regulatory sequence” is a structural gene which encodes products (e.g., transcription factors) which control the expression of other genes.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements include splicing signals, polyadenylation signals, termination signals, etc.

As used herein, the term “peptide transcription factor binding site” or “transcription factor binding site” refers to a nucleotide sequence which binds protein transcription factors and, thereby, controls some aspect of the expression of nucleic acid sequences. For example, Sp-1 and AP1 (activator protein 1) binding sites are examples of peptide transcription factor binding sites.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, post-transcriptional cleavage and polyadenylation.

A “non-human animal” refers to any animal which is not a human and includes vertebrates such as rodents, non-human primates, ovines, bovines, ruminants, lagomorphs, porcines, caprines, equines, canines, felines, aves, etc. Preferred non-human animals are selected from the order Rodentia. “Non-human animal” additionally refers to amphibians (e.g., Xenopus), reptiles, insects (e.g., Drosophila) and other non-mammalian animal species.

As used herein, the term “transgenic” refers to an organism or cell that has DNA derived from another organism inserted into which becomes integrated into the genome either of somatic and/or germ line cells of the plant or animal. A “transgene” means a DNA sequence which is partly or entirely heterologous (i.e., not present in nature) to the plant or animal in which it is found, or which is homologous to an endogenous sequence (i.e., a sequence that is found in the animal in nature) and is inserted into the plant' or animal's genome at a location which differs from that of the naturally occurring sequence. Transgenic plants or animals which include one or more transgenes are within the scope of this disclosure. Additionally, a “transgenic” as used herein refers to an organism that has had one or more genes modified and/or “knocked out” (made non-functional or made to function at reduced level, i.e., a “knockout” mutation) by the disclosure's methods, by homologous recombination, TFO mutation or by similar processes. For example, in some embodiments, a transgenic organism or cell includes inserted DNA that includes a foreign promoter and/or coding region.

A “transformed cell” is a cell or cell line that has acquired the ability to grow in cell culture for multiple generations, the ability to grow in soft agar, and/or the ability to not have cell growth inhibited by cell-to-cell contact. In this regard, transformation refers to the introduction of foreign genetic material into a cell or organism. Transformation may be accomplished by any method known which permits the successful introduction of nucleic acids into cells and which results in the expression of the introduced nucleic acid. “Transformation” includes but is not limited to such methods as transfection, microinjection, electroporation, nucleofection and lipofection (liposome-mediated gene transfer). Transformation may be accomplished through use of any expression vector. For example, the use of baculovirus to introduce foreign nucleic acid into insect cells is contemplated. The term “transformation” also includes methods such as P-element mediated germline transformation of whole insects. Additionally, transformation refers to cells that have been transformed naturally, usually through genetic mutation.

As used herein “exogenous” means that the gene encoding the protein is not normally expressed in the cell. Additionally, “exogenous” refers to a gene transfected into a cell to augment the normal (i.e., natural) level of expression of that gene.

A peptide sequence and nucleotide sequence may be “endogenous” or “heterologous” (i.e., “foreign”). The term “endogenous” refers to a sequence which is naturally found in the cell into which it is introduced so long as it does not contain some modification relative to the naturally occurring sequence. The term “heterologous” refers to a sequence which is not endogenous to the cell into which it is introduced. For example, heterologous DNA includes a nucleotide sequence which is ligated to, or is manipulated to become ligated to, a nucleic acid sequence to which it is not ligated in nature, or to which it is ligated at a different location in nature. Heterologous DNA also includes a nucleotide sequence which is naturally found in the cell into which it is introduced, and which contains some modification relative to the naturally occurring sequence. Generally, although not necessarily, heterologous DNA encodes heterologous RNA and heterologous proteins that are not normally produced by the cell into which it is introduced. Examples of heterologous DNA include reporter genes, transcriptional and translational regulatory sequences, DNA sequences which encode selectable marker proteins (e.g., proteins which confer drug resistance), etc.

In certain aspects and embodiments of the disclosures herein, provided are methods for introducing a gene repair oligonucleobase (GRON)-mediated mutation into a target deoxyribonucleic acid (DNA) sequence in a plant cell; for example, for the purpose of modifying an ACCase gene such as provided herein. In certain embodiments the methods may include, inter alia, culturing the plant cell under conditions that increase one or more cellular DNA repair processes prior to, and/or coincident with, delivery of a GRON into the plant cell; and/or delivery of a GRON into the plant cell greater than 15 bases in length, the GRON optionally comprising one or more; or two or more; mutation sites (such as ACCase mutation sites as provided herein) for introduction into the target DNA.

A “gene repair oligonucleotide” or “GRON” as used herein means an oligonucleobase (e.g., mixed duplex oligonucleotides, non-nucleotide containing molecules, single stranded oligodeoxynucleotides, double stranded oligodeoxynucleotides and other gene repair molecules) that can under certain conditions direct single, or in some embodiments multiple, nucleotide deletions, insertions or substitutions in a DNA sequence. This oligonucleotide-mediated gene repair editing of the genome may comprise both non-homology-based repair systems (e.g., non-homologous end joining) and homology-based repair systems (e.g., homology-directed repair). The GRON is typically designed to align in register with a genomic target except for the designed mismatch(es). These mismatches can be recognized and corrected by harnessing one or more of the cell's endogenous DNA repair systems. In some embodiments a GRON or oligonucleotide can be designed to contain multiple differences when compared to the organism's target sequence. These differences may not all affect the protein sequence translated from said target sequence and in one or more cases be known as silent changes. Numerous variations of GRON structure, chemistry and function are described elsewhere herein. In various embodiments, a GRON as used herein may have one or more modifications. For example, a GRON as used herein may have one or more modifications that attract DNA repair machinery to the targeted (mismatch) site and/or that prevent recombination of part or all of the GRON (other than the desired targeted deletions, insertions, substitutions or the like) into the genomic DNA of the target DNA sequence and/or that increase the stability of the GRON.

In various embodiments, a GRON may have both RNA and DNA nucleotides and/or other types of nucleobases. In some embodiments, one or more of the DNA or RNA nucleotides comprise a modification.

In one aspect, provided is a method of causing a genetic change in a plant cell (for example a genetic change in an ACCase gene), wherein the method involves exposing the cell to a DNA cutter and a GRON, for example a GRON that is modified as contemplated herein. In some embodiments the GRON may be modified such as with a Cy3 group, 3PS group, a 2′O-methyl group or other modification such as contemplated herein. In another aspect, provided is a plant cell that includes a DNA cutter and a GRON (such as a GRON that binds and/or modifies an ACCase gene), for example where the GRON is modified such as with a Cy3 group, 3PS group, a 2′O-methyl group or other modification. In some embodiments, the DNA cutter is one or more selected from a CRISPR, which includes but is not limited to Cas9, Cpf1 and their corresponding homologues, orthologues and/or paralogues, a base editor, a TALEN, a zinc finger, meganuclease, and a DNA-cutting antibiotic. In some embodiments, the DNA cutter is a CRISPR which includes but is not limited to Cas9, Cpf1 and their corresponding homologues, orthologues and/or paralogues, a base editor. In some embodiments, the DNA cutter is a TALEN. The DNA cutter can be plasmid (DNA) based, RNA and/or protein. In some embodiments, the GRON is between 15 and 60 nucleobases in length; or between 30 and 40 nucleobases in length; or between 35 and 45 nucleobases in length; or between 20 and 70 nucleobases in length; or between 20 and 200 nucleobases in length; or between 30 and 180 nucleobases in length; or between 50 and 160 nucleobases in length; or between 70 and 150 nucleobases in length; or between 80 and 120 nucleobases in length; or between 90 and 110 nucleobases in length; or between 95 and 105 nucleobases in length; or between 80 and 300 nucleobases in length; or between 90 and 250 nucleobases in length; or between 100 and 150 nucleobases in length; or between 100 and 300 nucleobases in length; or between 150 and 200 nucleobases in length; or between 200 and 300 nucleobases in length; or between 250 and 350 nucleobases in length; or between 50 and 110 nucleobases in length; or between 50 and 200 nucleobases in length; or between 150 and 210 nucleobases in length; or between 20 and 1000 nucleobases in length; or between 100 and 1000 nucleobases in length; or between 200 and 1000 nucleobases in length; or between 300 and 1000 nucleobases in length; or between 400 and 1000 nucleobases in length; or between 500 and 1000 nucleobases in length; or between 600 and 1000 nucleobases in length; or between 700 and 1000 nucleobases in length; or between 800 and 1000 nucleobases in length; or between 900 and 1000 nucleobases in length; or between 300 and 800 nucleobases in length; or between 400 and 600 nucleobases in length; or between 500 and 700 nucleobases in length; or between 600 and 800 nucleobases in length; or longer than 30 nucleobases in length; or longer than 35 nucleobases in length; or longer than 40 nucleobases in length; or longer than 50 nucleobases in length; or longer than 60 nucleobases in length; or longer than 65 nucleobases in length; or longer than 70 nucleobases in length; or longer than 75 nucleobases in length; or longer than 80 nucleobases in length; or longer than 85 nucleobases in length; or longer than 90 nucleobases in length; or longer than 95 nucleobases in length; or longer than 100 nucleobases in length; or longer than 110 nucleobases in length; or longer than 125 nucleobases in length; or longer than 150 nucleobases in length; or longer than 165 nucleobases in length; or longer than 175 nucleobases in length; or longer than 200 nucleobases in length; or longer than 250 nucleobases in length; or longer than 300 nucleobases in length; or longer than 350 nucleobases in length; or longer than 400 nucleobases in length; or longer than 450 nucleobases in length; or longer than 500 nucleobases in length; or longer than 550 nucleobases in length; or longer than 600 nucleobases in length; or longer than 700 nucleobases in length; or longer than 800 nucleobases in length; or longer than 900 nucleobases in length.

GRONs may be targeted at both non-coding (NC) and coding (C) regions of a target gene.

The term “CRISPR” as used herein refers to elements; i.e., a Cas (CRISPR associated) gene, transcript (e.g., mRNA) and/or protein and at least one CRISPR spacer sequence (Clustered Regularly Interspaced Short Palindromic Repeats, also known as SPIDRs—SPacer Interspersed Direct Repeats); that when effectively present or expressed in a cell could effect cleavage of a target DNA sequence via CRISPR/Cas cellular machinery such as described in e.g., Cong et al., Science, vol. 339 no 6121 pp. 819-823 (2013); Jinek et al, Science, vol. 337:816-821 (2013); Wang et al., RNA, vol. 14, pp. 903-913 (2008); Zhang et al., Plant Physiology, vol. 161, pp. 20-27 (2013); Zhang et al, PCT Application No. PCT/US2013/074743; and Charpentier et al., PCT Application No. PCT/US2013/032589. CRISPRs as contemplated herein may comprise any nuclease variant, analogue, homologue, or orthologue including, but not limited to Cas9, Cas12, Cpf1, and MADzymes such as MAD7 released by Inscripta, Inc. (www.inscripta.com). In some embodiments, such as for example a CRISPR for use in a eukaryotic cell, a CRISPR as contemplated herein may also include an additional element that includes a sequence for one or more functional nuclear localization signals. CRISPRs as contemplated herein can be expressed in, administered to and/or present in a cell (such as a plant cell) in any of many ways or manifestations. For example, a CRISPR as contemplated herein may include or involve one or more of a CRISPR on a plasmid, a CRISPR nickase on a plasmid, a CRISPRa on a plasmid, or a CRISPRi on a plasmid as follows:

CRISPR on a plasmid. A recombinant expression vector comprising:
(i) a nucleotide sequence encoding a DNA-targeting RNA (e.g., guide RNA), wherein the DNA-targeting RNA comprises:
   a. a first segment comprising a nucleotide sequence that is complementary to a sequence in a target DNA (e.g., protospacer, spacer, or crRNA); and
   b. a second segment that interacts with a site-directed modifying polypeptide (e.g., trans-activating crRNA or tracrRNA); and
(ii) a nucleotide sequence encoding the site-directed modifying polypeptide (e.g., Cas gene), wherein the site-directed polypeptide comprises:
   a. an RNA-binding portion that interacts with the DNA-targeting RNA (e.g., REC lobe); and
   b. an activity portion that causes double-stranded breaks within the target DNA (e.g., NUC lobe), wherein the site of the double-stranded breaks within the target DNA is determined by the DNA-targeting RNA.

CRISPR nickase on a plasmid. A recombinant expression vector comprising:
(i) a nucleotide sequence encoding a DNA-targeting RNA (e.g., guide RNA), wherein the DNA-targeting RNA comprises:
   a. a first segment comprising a nucleotide sequence that is complementary to a sequence in a target DNA (e.g., protospacer, spacer, or crRNA); and
   b. a second segment that interacts with a site-directed modifying polypeptide (e.g., trans-activating crRNA or tracrRNA); and
(ii) a nucleotide sequence encoding the site-directed modifying polypeptide (e.g., Cas gene), wherein the site-directed polypeptide comprises:
   a. an RNA-binding portion that interacts with the DNA-targeting RNA (e.g., REC lobe); and
   b. an activity portion that causes single-stranded breaks within the target DNA (e.g., NUC lobe), wherein the site of the single-stranded breaks within the target DNA is determined by the DNA-targeting RNA.

CRISPRa on a plasmid. A recombinant expression vector comprising:
(i) a nucleotide sequence encoding a DNA-targeting RNA (e.g., guide RNA), wherein the DNA-targeting RNA comprises:
   a. a first segment comprising a nucleotide sequence that is complementary to a sequence in a target DNA (e.g., protospacer, spacer, or crRNA); and
   b. a second segment that interacts with a site-directed modifying polypeptide (e.g., trans-activating crRNA or tracrRNA); and
(ii) a nucleotide sequence encoding the site-directed modifying polypeptide (e.g., Cas gene), wherein the site-directed polypeptide comprises:
   a. an RNA-binding portion that interacts with the DNA-targeting RNA (e.g., REC lobe); and
   b. an activity portion that modulates transcription (e.g., NUC lobe; in certain embodiments increases transcription) within the target DNA, wherein the site of the transcriptional modulation within the target DNA is determined by the DNA-targeting RNA.

CRISPRi on a plasmid. A recombinant expression vector comprising:
(i) a nucleotide sequence encoding a DNA-targeting RNA (e.g., guide RNA), wherein the DNA-targeting RNA comprises:
   a. a first segment comprising a nucleotide sequence that is complementary to a sequence in a target DNA (e.g., protospacer, spacer, or crRNA); and
   b. a second segment that interacts with a site-directed modifying polypeptide (e.g., trans-activating crRNA or tracrRNA); and
(ii) a nucleotide sequence encoding the site-directed modifying polypeptide (e.g., Cas gene), wherein the site-directed polypeptide comprises:
   a. an RNA-binding portion that interacts with the DNA-targeting RNA (e.g., REC lobe); and
   b. an activity portion that modulates transcription/translation (e.g., NUC lobe; in some embodiments decreases transcription/translation) within the target DNA, wherein the site of transcriptional/translational modulation within the target DNA is determined by the DNA-targeting RNA.

Each of the CRISPR on a plasmid, CRISPR nickase on a plasmid, CRISPRa on a plasmid, and CRISPRi on a plasmid may in some embodiments alternatively have one or more appropriate elements be administered, expressed or present in a cell as an RNA (e.g., mRNA) or a protein rather than on a plasmid. Delivery of protected mRNA may be as described in Kariko, et al, U.S. Pat. No. 8,278,036.

In some embodiments, each of the CRISPRi and CRISPRa may include a deactivated Cas9 (dCas9). A deactivated Cas9 still binds to target DNA but does not have cutting activity. Nuclease-deficient Cas9 can result from D10A and H840A point mutations which inactivates its two catalytic domains.

In some embodiments, a CRISPRi inhibits transcription initiation or elongation via steric hindrance of RNA Polymerase II. CRISPRi can optionally be enhanced (CRISPRei) by fusion of a strong repressor domain to the C-terminal end of a dCas9 protein. In some embodiments, a repressor domain recruits and employs chromatin modifiers. In some embodiments, the repressor domain may include, but is not limited to domains as described in Kagale et al., Epigenetics, vol. 6 no 2 pp 141-146 (2011):

1. OsERF3 repressor domain (“LxLxPP” motif)
(SEQ ID NO: 24)
LDLNRPPPVEN
2. AtBRD repressor domain (“R/KLFGV” motif)
(SEQ ID NO: 25)
LRLFGVNM
3. AtHsfB1 repressor domain (“R/KLFGV” motif)
(SEQ ID NO: 26)
LKLFGVWL
4. AtSUP repressor domain (“EAR” motif)
(SEQ ID NO: 27)
LDLELRLGFA
5. full AtSUP gene containing repressor domain
(“EAR” motif)
(SEQ ID NO: 28)
ERSNSIELRNSFYGRARTSPWSYGDYDNCQQDHDYLLGFSWPPRSY
TCSFCKREFRSAQALGGHMNVHRRDRARLRLQQSPSSSSTPSPPYP
NPNYSYSTMANSPPPHHSPLTLFPTLSPPSSPRYRAGLIRSLSPKS
KHTPENACKTKKSSLLVEAGEATRFTSKDACKILRNDEIISLELEI
GLINESEQDLDLELRLGFA*-

In some embodiments, a CRISPRa activation of transcription achieved by use of dCas9 protein containing a fused C-terminal end transcriptional activator. In some embodiments, an activation may include, but is not limited to VP64 (4× VP16), AtERF98 activation domain, or AtERF98×4 concatemers such as described in Cheng et al., Cell Research, pp 1-9 (2013); Perez-Pinera et al., Nature Methods, vol. 10 pp 913-976 (2013); Maeder et al., Nature Methods, vol. 10 pp 977-979 (2013) and Mali et al., Nature Biotech., vol. 31 pp 833-838 (2013).

In some embodiments the CRISPR includes a nickase. In certain embodiments, two or more CRISPR nickases are used. In some embodiments, the two or more nickases cut on opposite strands of target nucleic acid. In other embodiments, the two or more nickases cut on the same strand of target nucleic acid.

As used herein, “repressor protein” or “repressor” refers to a protein that binds to operator of DNA or to RNA to prevent transcription or translation, respectively.

As used herein, “repression” refers to inhibition of transcription or translation by binding of repressor protein to specific site on DNA or mRNA. In some embodiments, repression includes a significant change in transcription or translation level of at least 1.5-fold, in other embodiments at least 2-fold, and in other embodiments at least 5-fold.

As used herein, an “activator protein” or “activator” with regard to gene transcription and/or translation, refers to a protein that binds to operator of DNA or to RNA to enhance or increase transcription or translation, respectively.

As used herein with regard to gene transcription and/or translation, “activation” with regard to gene transcription and/or translation, refers to enhancing or increasing transcription or translation by binding of activator protein to specific site on DNA or mRNA. In some embodiments, activation includes a significant change in transcription or translation level of at least 1.5-fold, in some embodiments at least 2-fold, and in some embodiments at least 5-fold.

In certain embodiments, conditions that increase one or more cellular DNA repair processes may include one or more of: introduction of one or more sites into the GRON or into the plant cell DNA that are targets for base excision repair, introduction of one or more sites into the GRON or into the plant cell DNA that are targets for non-homologous end joining, introduction of one or more sites into the GRON or into the plant cell DNA that are targets for microhomology-mediated end joining, introduction of one or more sites into the GRON or into the plant cell DNA that are targets for homologous recombination, and introduction of one or more sites into the GRON or into the plant cell DNA that are targets for effecting repair (e.g., base-excision repair (BER); homologous recombination repair (HR); mismatch repair (MMR); non-homologous end-joining repair (NHEJ) which include classical and alternative NHEJ; and nucleotide excision repair (NER)).

As described herein, GRONs for use herein may include one or more of the following (non-limiting) alterations from conventional RNA and DNA nucleotides:
one or more abasic nucleotides;
one or more 8′oxo dA and/or 8′oxo dG nucleotides;
a reverse (or “inverted”) base (e.g., inverted dC (“idC”), dT (“idT”), dU (“idU”), dA (“idA”) or dG (“idG”)) at the 3′ end thereof;
one or more 2′O-methyl nucleotides;
one or more RNA nucleotides;
one or more RNA nucleotides at the 5′ end thereof, and in some embodiments 2, 3, 4, 5, 6, 7, 8, 9, 10, or more; wherein one or more of the RNA nucleotides may further be modified; one or more RNA nucleotides at the 3′ end thereof, and in some embodiments 2, 3, 4, 5, 6, 7, 8, 9, 10, or more; wherein one or more of the RNA nucleotides may further be modified;
one or more 2′O-methyl RNA nucleotides at the 5′ end thereof, and in some embodiments 2, 3, 4, 5, 6, 7, 8, 9, 10, or more;
an intercalating dye;
a 5′ terminus cap;
a backbone modification selected from the group consisting of a phosphothioate modification, a methyl phosphonate modification, a locked nucleic acid (LNA) modification, a O-(2-methoxyethyl) (MOE) modification, a diPS modification, and a peptide nucleic acid (PNA) modification;
one or more intrastrand crosslinks;
one or more fluorescent dyes conjugated thereto, and in some embodiments at the 5′ or 3′ end of the GRON; and
one or more bases which increase hybridization energy.

The term “wobble base” as used herein refers to a change in a one or more nucleotide bases of a reference nucleotide sequence wherein the change does not change the sequence of the amino acid coded by the nucleotide relative to the reference sequence.

The term “non-nucleotide” or “abasic nucleotide” as use herein refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine. It may have substitutions for a 2′ or 3′ H or OH as described in the art and herein.

As described herein, in certain embodiments GRON quality and conversion efficiency may be improved by synthesizing all or a portion of the GRON using nucleotide multimers, such as dimers, trimers, tetramers, etc. improving its purity.

In certain embodiments, the target deoxyribonucleic acid (DNA) sequence is within a plant cell, for example the target DNA sequence is in the plant cell genome. The plant cell may be non-transgenic or transgenic, and the target DNA sequence may be a transgene or an endogenous gene of the plant cell.

In certain embodiments, the conditions that increase one or more cellular DNA repair processes comprise introducing one or more compounds which induce single or double DNA strand breaks into the plant cell prior to, or coincident to, or after delivering the GRON into the plant cell. Exemplary compounds are described herein.

In certain embodiments, the methods further comprise regenerating a plant having a mutation introduced by the GRON from the plant cell and may comprise collecting seeds from the plant.

In related aspects, the present disclosure relates to plant cells comprising a genomic modification introduced by a GRON according to the methods described herein, a plant comprising a genomic modification introduced by a GRON according to the methods described herein, or a seed comprising a genomic modification introduced by a GRON according to the methods described herein; or progeny of a seed comprising a genomic modification introduced by a GRON according to the methods described herein.

### Constructs

The nucleic acid molecules disclosed herein (e.g., site specific nucleases, or guide RNA for CRISPRs) can be used in the production of recombinant nucleic acid constructs. In one embodiment, the nucleic acid molecules of the present disclosure can be used in the preparation of nucleic acid constructs, for example, expression cassettes for expression in the plant, microorganism, or animal of interest such as ACCase expression constructs optionally having one or more mutations as described herein. This expression may be transient for instance when the construct is not integrated into the host genome or maintained under the control offered by the promoter and the position of the construct within the host's genome if it becomes integrated.

Expression cassettes may include regulatory sequences operably linked to the site-specific nuclease or guide RNA sequences disclosed herein. The cassette may additionally contain at least one additional gene to be co-transformed into the organism. Alternatively, the additional gene or genes can be provided on multiple expression cassettes.

The nucleic acid constructs may be provided with a plurality of restriction sites for insertion of the site-specific nuclease coding sequence to be under the transcriptional regulation of the regulatory regions. The nucleic acid constructs may additionally contain nucleic acid molecules encoding for selectable marker genes.

Any promoter can be used in the production of the nucleic acid constructs. The promoter may be native or analogous, or foreign or heterologous, to the plant, microbial, or animal host nucleic acid sequences disclosed herein. Additionally, the promoter may be the natural sequence or alternatively a synthetic sequence. Where the promoter is “foreign” or “heterologous” to the plant, microbial, or animal host, it is intended that the promoter is not found in the native plant, microbial, or animal into which the promoter is introduced. As used herein, a chimeric gene comprises a coding sequence operably linked to a transcription initiation region that is heterologous to the coding sequence.

The site directed nuclease sequences disclosed herein may be expressed using heterologous promoters.

Any promoter can be used in the preparation of constructs to control the expression of the site directed nuclease sequences, such as promoters providing for constitutive, tissue-preferred, inducible, or other promoters for expression in plants, microbes, or animals. Constitutive promoters include, for example, the core promoter of the Rsyn7 promoter and other constitutive promoters disclosed in WO 99/43 838 and U.S. Pat. No. 6,072,050; the core CaMV 35S promoter (Odell et al. Nature 313:810-812; 1985); rice actin (McElroy et al., Plant Cell 2:163-171, 1990); ubiquitin (Christensen et al., Plant Mol. Biol. 12:619-632, 1989 and Christensen et al., Plant Mol. Biol. 18:675-689, 1992); pEMU (Last et al., Theor. Appl. Genet. 81:581-588, 1991); MAS (Velten et al., EMBO J. 3:2723-2730, 1984); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to direct site directed nuclease expression within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al., Plant J. 12 (2): 255-265, 1997; Kawamata et al., Plant Cell Physiol. 38 (7): 792-803, 1997; Hansen et al., Mol. Gen Genet. 254 (3): 337-343, 1997; Russell et al., Transgenic Res. 6 (2): 157-168, 1997; Rinehart et al., Plant Physiol. 1 12 (3): 1331-1341, 1996; Van Camp et al., Plant Physiol. 1 12 (2): 525-535, 1996; Canevascini et al., Plant Physiol. 112 (2): 513-524, 1996; Yamamoto et al., Plant Cell Physiol. 35 (5): 773-778, 1994; Lam, Results Probl. Cell Differ. 20:181-196, 1994; Orozco et al. Plant Mol Biol. 23 (6): 1129-1138, 1993; Matsuoka et al., Proc Nat'l. Acad. Sci. U.S.A. 90 (20): 9586-9590, 1993; and Guevara-Garcia et al., Plant J. 4 (3): 495-505, 1993.

The nucleic acid constructs may also include transcription termination regions. Where transcription terminations regions are used, any termination region may be used in the preparation of the nucleic acid constructs. For example, the termination region may be derived from another source (i.e., foreign or heterologous to the promoter). Examples of termination regions that are available for use in the constructs of the present disclosure include those from the Ti-plasmid of A. tumefaciens, such as the octopine synthase and nopaline synthase termination regions. See also Guerineau et al., Mol. Gen. Genet. 262:141-144, 1991; Proudfoot, Cell 64:671-674, 1991; Sanfacon et al., Genes Dev. 5:141-149, 1991; Mogen et al., Plant Cell 2:1261-1272, 1990; Munroe et al., Gene 91:151-158, 1990; Ballas et al., Nucleic Acids Res. 17:7891-7903, 1989; and Joshi et al., Nucleic Acid Res. 15:9627-9639, 1987.

In conjunction with any of the aspects, embodiments, methods and/or compositions disclosed herein, the nucleic acids may be optimized for increased expression in the transformed plant. That is, the nucleic acids encoding the site directed nuclease proteins can be synthesized using plant-preferred codons for improved expression. See, for example, Campbell and Gowri, (Plant Physiol. 92:1-11, 1990) for a discussion of host-preferred codon usage. Methods are available in the art for synthesizing plant-preferred genes. See, for example, U.S. Pat. Nos. 5,380,831, and 5,436,391, and Murray et al., Nucleic Acids Res. 17:477-498, 1989. See also e.g., Lanza et al., BMC Systems Biology 8:33-43, 2014; Burgess-Brown et al., Protein Expr. Purif. 59:94-102, 2008; Gustafsson et al., Trends Biotechnol 22:346-353, 2004.

In addition, other sequence modifications can be made to the nucleic acid sequences disclosed herein. For example, additional sequence modifications are known to enhance gene expression in a cellular host. These include elimination of sequences encoding spurious polyadenylation signals, exon/intron splice site signals, transposon-like repeats, and other such well-characterized sequences that may be deleterious to gene expression. The G-C content of the sequence may also be adjusted to levels average for a target cellular host, as calculated by reference to known genes expressed in the host cell. In addition, the sequence can be modified to avoid predicted hairpin secondary mRNA structures.

Other nucleic acid sequences may also be used in the preparation of the constructs of the present disclosure, for example to enhance the expression of the site directed nuclease coding sequence. Such nucleic acid sequences include the introns of the maize AdhI, intronl gene (Callis et al., Genes and Development 1:1183-1200, 1987), and leader sequences, (W-sequence) from the Tobacco Mosaic Virus (TMV), Maize Chlorotic Mottle Virus and Alfalfa Mosaic Virus (Gallie et al., Nucleic Acid Res. 15:8693-8711, 1987; and Skuzeski et al., Plant Mol. Biol. 15:65-79, 1990). The first intron from the shrunken-1 locus of maize has been shown to increase expression of genes in chimeric gene constructs. U.S. Pat. Nos. 5,424,412 and 5,593,874 disclose the use of specific introns in gene expression constructs, and Gallie et al. (Plant Physiol. 106:929-939, 1994) also have shown that introns are useful for regulating gene expression on a tissue specific basis. To further enhance or to optimize site directed nuclease gene expression, the plant expression vectors disclosed herein may also contain DNA sequences containing matrix attachment regions (MARs). Plant cells transformed with such modified expression systems, then, may exhibit overexpression or constitutive expression of a nucleotide sequence of the disclosure.

The expression constructs disclosed herein can also include nucleic acid sequences capable of directing the expression of the site directed nuclease sequence to the chloroplast or other organelles and structures in both prokaryotes and eukaryotes. Such nucleic acid sequences include chloroplast targeting sequences that encodes a chloroplast transit peptide to direct the gene product of interest to plant cell chloroplasts. Such transit peptides are known in the art. With respect to chloroplast-targeting sequences, “operably linked” means that the nucleic acid sequence encoding a transit peptide (i.e., the chloroplast-targeting sequence) is linked to the site directed nuclease nucleic acid molecules disclosed herein such that the two sequences are contiguous and in the same reading frame. See, for example, Von Heijne et al., Plant Mol. Biol. Rep. 9:104-126, 1991; Clark et al., J. Biol. Chem. 264:17544-17550, 1989; Della-Cioppa et al., Plant Physiol. 84:965-968, 1987; Romer et al., Biochem. Biophys. Res. Commun. 196:1414-1421, 1993; and Shah et al., Science 233:478-481, 1986.

Chloroplast targeting sequences are known in the art and include the chloroplast small subunit of ribulose-1,5-bisphosphate carboxylase (Rubisco) (de Castro Silva Filho et al., Plant Mol. Biol. 30:769-780, 1996; Schnell et al., J. Biol. Chem. 266 (5): 3335-3342, 1991); 5-(enolpyruvyl) shikimate-3-phosphate synthase (EPSPS) (Archer et al., J. Bioenerg. Biomemb. 22 (6): 789-810, 1990); tryptophan synthase (Zhao et al., J. Biol. Chem. 270 (1 1): 6081-6087, 1995); plastocyanin (Lawrence et al., J. Biol. Chem. 272 (33): 20357-20363, 1997); chorismate synthase (Schmidt et al., J. Biol. Chem. 268 (36): 27447-27457, 1993); and the light harvesting chlorophyll a/b binding protein (LHBP) (Lamppa et al., J. Biol. Chem. 263:14996-14999, 1988). See also Von Heijne et al., Plant Mol. Biol. Rep. 9:104-126, 1991; Clark et al., J. Biol. Chem. 264:17544-17550, 1989; Della-Cioppa et al., Plant Physiol. 84:965-968, 1987; Romer et al., Biochem. Biophys. Res. Commun. 196:1414-1421, 1993; and Shah et al., Science 233:478-481, 1986.

In conjunction with any of the aspects, embodiments, methods and/or compositions disclosed herein, the nucleic acid constructs may be prepared to direct the expression of the mutant site directed nuclease coding sequence from the plant cell chloroplast. Methods for transformation of chloroplasts are known in the art. See, for example, Svab et al., Proc. Nat'l. Acad. Sci. U.S.A. 87:8526-8530, 1990; Svab and Maliga, Proc. Nat'l. Acad. Sci. U.S.A. 90:913-917, 1993; Svab and Maliga, EMBO J. 12:601-606, 1993. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. Proc. Nat′1. Acad. Sci. U.S.A. 91:7301-7305, 1994.

The nucleic acids of interest to be targeted to the chloroplast may be optimized for expression in the chloroplast to account for differences in codon usage between the plant nucleus and this organelle. In this manner, the nucleic acids of interest may be synthesized using chloroplast-preferred codons. See, for example, U.S. Pat. No. 5,380,831, herein incorporated by reference.

The nucleic acid constructs can be used to transform plant cells and regenerate transgenic plants comprising the site directed nuclease coding sequences. Numerous plant transformation vectors and methods for transforming plants are available. See, for example, U.S. Pat. No. 6,753,458, An et al., Plant Physiol., 81:301-305, 1986; Fry et al., Plant Cell Rep. 6:321-325, 1987; Block Theor. Appl Genet. 76:767-774, 1988; Hinchee et al., Stadler. Genet. Symp. 203212.203-212, 1990; Cousins et al., Aust. J. Plant Physiol. 18:481-494, 1991; Chee and Slightom Gene. 118:255-260, 1992; Christou et al., Trends. Biotechnol. 10:239-246, 1992; D'Halluin et al., Bio/Technol. 10:309-3 14, 1992; Dhir et al., Plant Physiol. 99:81-88, 1992; Casas et al., Proc. Nat'l. Acad Sci. U.S.A. 90:11212-11216, 1993; Christou, P., In Vitro Cell. Dev. Biol.-Plant 29P: 119-124, 1993; Davies, et al., Plant Cell Rep. 12:180-183, 1993; Dong and Mc Hughen Plant Sci. 91:139-148, 1993; Franklin Trieu Cassidy Dixon Nelson 1993, Plant Cell Report 12, 74-79; Golovkin et al., Plant Sci. 90:41-52, 1993; Guo Chin Sci. Bull. 38:2072-2078; Asano et al., Plant Cell Rep. 13, 1994; Ayeres and Park Crit. Rev. Plant. Sci. 13:219-239, 1994; Barcelo et al., Plant. J. 5:583-592, 1994; Becker, et al., Plant. J. 5:299-307, 1994; Borkowska et al., Acta. Physiol Plant. 16:225-230, 1994; Christou Agro. Food. Ind. Hi Tech. 5:17-27, 1994; Eapen et al., Plant Cell Rep. 13:582-586, 1994; Hartman et al., Bio-Technology 12:919923, 1994; Ritala et al., Plant. Mol. Biol. 24:317-325, 1994; and Wan and Lemaux Plant Physiol. 104:3748, 1994. The constructs may also be transformed into plant cells using homologous recombination.

The term “wild-type” when made in reference to a peptide sequence and nucleotide sequence refers to a peptide sequence and nucleotide sequence (locus/gene/allele), respectively, which has the characteristics of that peptide sequence and nucleotide sequence when isolated from a naturally occurring source. A wild-type peptide sequence and nucleotide sequence is that which is most frequently observed in a population and is thus arbitrarily designated the “normal” or “wild-type” form of the peptide sequence and nucleotide sequence, respectively. “Wild-type” may also refer to the sequence at a specific nucleotide position or positions, or the sequence at a particular codon position or positions, or the sequence at a particular amino acid position or positions.

“Consensus sequence” is defined as a sequence of amino acids or nucleotides that contain identical amino acids or nucleotides or functionally equivalent amino acids or nucleotides for at least 25% of the sequence. The identical or functionally equivalent amino acids or nucleotides need not be contiguous.

A nucleobase is a base, which in certain preferred embodiments is a purine, pyrimidine, or a derivative or analog thereof. Nucleosides are nucleobases that contain a pentosefuranosyl moiety, e.g., an optionally substituted riboside or 2′-deoxyriboside. Nucleosides can be linked by one of several linkage moieties, which may or may not contain phosphorus. Nucleosides that arc linked by unsubstituted phosphodiester linkages are termed nucleotides. The term “nucleobase” as used herein includes peptide nucleobases, the subunits of peptide nucleic acids, and morpholine nucleobases as well as nucleosides and nucleotides.

An oligonucleobase is a polymer comprising nucleobases; in some embodiments at least a portion of which can hybridize by Watson-Crick base pairing to a DNA having the complementary sequence. An oligonucleobase chain may have a single 5′ and 3′ terminus, which are the ultimate nucleobases of the polymer. A particular oligonucleobase chain can contain nucleobases of all types. An oligonucleobase compound is a compound comprising one or more oligonucleobase chains that may be complementary and hybridized by Watson-Crick base pairing. Ribo-type nucleobases include pentosefuranosyl containing nucleobases wherein the 2′ carbon is a methylene substituted with a hydroxyl, alkyloxy or halogen. Deoxyribo-type nucleobases are nucleobases other than ribo-type nucleobases and include all nucleobases that do not contain a pentosefuranosyl moiety.

In certain embodiments, an oligonucleobase strand may include both oligonucleobase chains and segments or regions of oligonucleobase chains. An oligonucleobase strand may have a 3′ end and a 5′ end, and when an oligonucleobase strand is coextensive with a chain, the 3′ and 5′ ends of the strand are also 3′ and 5′ termini of the chain.

As used herein the term “codon” refers to a sequence of three adjacent nucleotides (either RNA or DNA) constituting the genetic code that determines the insertion of a specific amino acid in a polypeptide chain during protein synthesis or the signal to stop protein synthesis. The term “codon” is also used to refer to the corresponding (and complementary) sequences of three nucleotides in the messenger RNA into which the original DNA is transcribed.

As used herein, the term “homology” refers to sequence similarity among proteins and DNA. The term “homology” or “homologous” refers to a degree of identity. There may be partial homology or complete homology. A partially homologous sequence is one that has less than 100% sequence identity when compared to another sequence.

“Heterozygous” refers to having different alleles at one or more genetic loci in homologous chromosome segments. As used herein “heterozygous” may also refer to a sample, a cell, a cell population or an organism in which different alleles at one or more genetic loci may be detected. Heterozygous samples may also be determined via methods known in the art such as, for example, nucleic acid sequencing. For example, if a sequencing electropherogram shows two peaks at a single locus and both peaks are roughly the same size, the sample may be characterized as heterozygous. Or, if one peak is smaller than another, but is at least about 25% the size of the larger peak, the sample may be characterized as heterozygous. In some embodiments, the smaller peak is at least about 15% of the larger peak. In other embodiments, the smaller peak is at least about 10% of the larger peak. In other embodiments, the smaller peak is at least about 5% of the larger peak. In other embodiments, a minimal amount of the smaller peak is detected.

As used herein, “homozygous” refers to having identical alleles at one or more genetic loci in homologous chromosome segments. “Homozygous” may also refer to a sample, a cell, a cell population or an organism in which the same alleles at one or more genetic loci may be detected. Homozygous samples may be determined via methods known in the art, such as, for example, nucleic acid sequencing. For example, if a sequencing electropherogram shows a single peak at a particular locus, the sample may be termed “homozygous” with respect to that locus.

The term “hemizygous” refers to a gene or gene segment being present only once in the genotype of a cell or an organism because the second allele is deleted or is not present on the homologous chromosome segment. As used herein “hemizygous” may also refer to a sample, a cell, a cell population or an organism in which an allele at one or more genetic loci may be detected only once in the genotype.

The term “zygosity status” as used herein refers to a sample, a cell population, or an organism as appearing heterozygous, homozygous, or hemizygous as determined by testing methods known in the art and described herein. The term “zygosity status of a nucleic acid” means determining whether the source of nucleic acid appears heterozygous, homozygous, or hemizygous. The “zygosity status” may refer to differences in at a single nucleotide position in a sequence. In some methods, the zygosity status of a sample with respect to a single mutation may be categorized as homozygous wild-type, heterozygous (i.e., one wild-type allele and one mutant allele), homozygous mutant, or hemizygous (i.e., a single copy of either the wild-type or mutant allele).

As used herein, the term “RTDS” refers to the Rapid Trait Development System™ (RTDS®) developed by Cibus. RTDS is a suite of technologies enabling site-specific gene modification using a system that is effective at making precise changes in a gene sequence without the incorporation of foreign genes or control sequences. Site-specific gene modification is followed by regeneration of cells with these precise changes into plants bearing these changes.

The term “about” as used herein means in quantitative terms plus or minus 10%. For example, “about 3%” would encompass 2.7-3.3% and “about 10%” would encompass 9-11%. Moreover, where “about” is used herein in conjunction with a quantitative term it is understood that in addition to the value plus or minus 10%, the exact value of the quantitative term is also contemplated and described. For example, the term “about 3%” expressly contemplates, describes and includes exactly 3%.

### RTDS and Repair Oligonucleotides (GRONs)

Various aspects and embodiments of the methods and compositions contemplated herein include methods to improve the efficiency of the targeting of modifications to specific locations in genomic or other nucleotide sequences (for example modifications to an ACCase gene such as contemplated herein).

RTDS in some embodiments is based on altering a targeted gene by utilizing the cell's own gene repair system to specifically modify the gene sequence in situ and not insert foreign DNA and gene expression control sequences. This procedure can effect a precise change in the genetic sequence while the rest of the genome is left unaltered. In some embodiments, in contrast to conventional transgenic GMOs, there is no integration of foreign genetic material, nor is any foreign genetic material left in the plant. The changes in the genetic sequence introduced by RTDS are not randomly inserted. Since affected genes remain in their native location, no random, uncontrolled or adverse pattern of expression occurs.

The molecule that effects this change is a chemically synthesized oligonucleotide (GRON) as described herein which may be composed of both DNA and modified RNA bases as well as other chemical moieties and is designed to hybridize at the targeted gene location to create or make a mismatched base-pair. This mismatched base-pair acts as a signal to attract the cell's own natural gene repair system to that site and correct (replace, insert or delete) the designated nucleotide or nucleotides within the gene. Once the correction process is complete the GRON molecule is degraded, the now modified or repaired gene is expressed under that gene's normal endogenous control mechanisms.

The methods and compositions disclosed herein can be practiced or made with “gene repair oligonucleobases” (GRON) having the conformations and chemistries as described in detail herein and below. The “gene repair oligonucleobases” as contemplated herein have also been described in published scientific and patent literature using other names including “recombinagenic oligonucleobases;” “RNA/DNA chimeric oligonucleotides;” “chimeric oligonucleotides;” “mixed duplex oligonucleotides” (MDONs); “RNA DNA oligonucleotides (RDOs);” “gene targeting oligonucleotides;” “genoplasts;” “single stranded modified oligonucleotides;” “Single stranded oligodeoxynucleotide mutational vectors” (SSOMVs); “duplex mutational vectors;” and “heteroduplex mutational vectors.” The gene repair oligonucleobase can be introduced into a plant cell using any method commonly used in the art, including but not limited to, microcarriers (biolistic delivery), microfibers, polyethylene glycol (PEG)-mediated uptake, electroporation, and microinjection.

In one embodiment, the gene repair oligonucleobase is a mixed duplex oligonucleotides (MDON) in which the RNA-type nucleotides of the mixed duplex oligonucleotide are made RNase resistant by replacing the 2′-hydroxyl with a fluoro, chloro or bromo functionality or by placing a substituent on the 2′-O. Suitable substituents include the substituents taught by the Kmiec II. Alternative substituents include the substituents taught by U.S. Pat. No. 5,334,711 (Sproat) and the substituents taught by patent publications EP 629 387 and EP 679 657 (collectively, the Martin Applications), which are hereby incorporated by reference. As used herein, a 2′-fluoro, chloro or bromo derivative of a ribonucleotide or a ribonucleotide having a T-OH substituted with a substituent described in the Martin Applications or Sproat is termed a “T-substituted ribonucleotide.” As used herein the term “RNA-type nucleotide” means a T-hydroxyl or 2′-substituted nucleotide that is linked to other nucleotides of a mixed duplex oligonucleotide by an unsubstituted phosphodiester linkage or any of the non-natural linkages taught by Kmiec I or Kmiec II. As used herein the term “deoxyribo-type nucleotide” means a nucleotide having a T-H, which can be linked to other nucleotides of a gene repair oligonucleobase by an unsubstituted phosphodiester linkage or any of the non-natural linkages taught by Kmiec I or Kmiec II.

In particular embodiments of the present disclosure, the gene repair oligonucleobase may be a mixed duplex oligonucleotide (MDON) that is linked solely by unsubstituted phosphodiester bonds. In alternative embodiments, the linkage is by substituted phosphodiesters, phosphodiester derivatives and non-phosphorus-based linkages as taught by Kmiec II. In yet another embodiment, each RNA-type nucleotide in the mixed duplex oligonucleotide is a 2′-Substituted Nucleotide. Particular preferred embodiments of 2′-Substituted Ribonucleotides are 2′-fluoro, T-methoxy, 2′-propyloxy, 2′-allyloxy, 2′-hydroxylethyloxy, 2′-methoxyethyloxy, T-fluoropropyloxy and 2′-trifluoropropyloxy substituted ribonucleotides. More preferred embodiments of 2′-Substituted Ribonucleotides are 2′-fluoro, 2′-methoxy, 2′-methoxyethyloxy, and 2′-allyloxy substituted nucleotides. In another embodiment the mixed duplex oligonucleotide is linked by unsubstituted phosphodiester bonds.

Although mixed duplex oligonucleotides (MDONs) having only a single type of 2′-substituted RNA-type nucleotide are more conveniently synthesized, the methods of the disclosure can be practiced with mixed duplex oligonucleotides having two or more types of RNA-type nucleotides. The function of an RNA segment may not be affected by an interruption caused by the introduction of a deoxynucleotide between two RNA-type trinucleotides, accordingly, the term RNA segment encompasses terms such as “interrupted RNA segment.” An uninterrupted RNA segment is termed a contiguous RNA segment. In an alternative embodiment an RNA segment can contain alternating RNase-resistant and unsubstituted 2′-OH nucleotides. The mixed duplex oligonucleotides in some embodiments have fewer than 100 nucleotides and other embodiments fewer than 85 nucleotides, but more than 50 nucleotides. The first and second strands are Watson-Crick base paired. In one embodiment the strands of the mixed duplex oligonucleotide are covalently bonded by a linker, such as a single stranded hexa, penta or tetranucleotide so that the first and second strands are segments of a single oligonucleotide chain having a single 3′ and a single 5′ end. The 3′ and 5′ ends can be protected by the addition of a “hairpin cap” whereby the 3′ and 5′ terminal nucleotides are Watson-Crick paired to adjacent nucleotides. A second hairpin cap can, additionally, be placed at the junction between the first and second strands distant from the 3′ and 5′ ends, so that the Watson-Crick pairing between the first and second strands is stabilized.

The first and second strands contain two regions that are homologous with two fragments of the target gene/allele, i.e., have the same sequence as the target gene/allele. A homologous region contains the nucleotides of an RNA segment and may contain one or more DNA-type nucleotides of connecting DNA segment and may also contain DNA-type nucleotides that are not within the intervening DNA segment. The two regions of homology are separated by, and each is adjacent to, a region having a sequence that differs from the sequence of the target gene, termed a “heterologous region.” The heterologous region can contain one, two or three mismatched nucleotides. The mismatched nucleotides can be contiguous or alternatively can be separated by one or two nucleotides that are homologous with the target gene/allele. Alternatively, the heterologous region can also contain an insertion or one, two, three or of five or fewer nucleotides. Alternatively, the sequence of the mixed duplex oligonucleotide may differ from the sequence of the target gene/allele only by the deletion of one, two, three, or five or fewer nucleotides from the mixed duplex oligonucleotide. The length and position of the heterologous region is, in this case, deemed to be the length of the deletion, even though no nucleotides of the mixed duplex oligonucleotide are within the heterologous region. The distance between the fragments of the target gene that are complementary to the two homologous regions is identical to the length of the heterologous region where a substitution or substitutions is intended. When the heterologous region contains an insertion, the homologous regions are thereby separated in the mixed duplex oligonucleotide farther than their complementary homologous fragments are in the gene/allele, and the converse is applicable when the heterologous region encodes a deletion.

The RNA segments of the mixed duplex oligonucleotides are each a part of a homologous region, i.e., a region that is identical in sequence to a fragment of the target gene, which segments together in some embodiments contain at least 13 RNA-type nucleotides and in some embodiments from 16 to 25 RNA-type nucleotides or yet other embodiments 18-22 RNA-type nucleotides or in some embodiments 20 nucleotides. In one embodiment, RNA segments of the homology regions are separated by and adjacent to, i.e., “connected by” an intervening DNA segment. In one embodiment, each nucleotide of the heterologous region is a nucleotide of the intervening DNA segment. An intervening DNA segment that contains the heterologous region of a mixed duplex oligonucleotide is termed a “mutator segment.”

In another embodiment of the methods and compositions of the present disclosure, a gene repair oligonucleobase (GRON) is a single stranded oligodeoxynucleotide mutational vector (SSOMV), such as disclosed in International Patent Application PCT/USOO/23457, U.S. Pat. Nos. 6,271,360, 6,479,292, and 7,060,500 which is incorporated by reference in its entirety. The sequence of the SSOMV is based on the same principles as the mutational vectors described in U.S. Pat. Nos. 5,756,325; 5,871,984; 5,760,012; 5,888,983; 5,795,972; 5,780,296; 5,945,339; 6,004,804; and 6,010,907 and in International Publication Nos. WO 98/49350; WO 99/07865; WO 99/58723; WO 99/58702; and WO 99/40789. The sequence of the SSOMV contains two regions that are homologous with the target sequence separated by a region that contains the desired genetic alteration termed the mutator region. The mutator region can have a sequence that is the same length as the sequence that separates the homologous regions in the target sequence but having a different sequence. Such a mutator region can cause a substitution. Alternatively, the homologous regions in the SSOMV can be contiguous to each other, while the regions in the target gene having the same sequence are separated by one, two or more nucleotides. Such an SSOMV causes a deletion from the target gene of the nucleotides that are absent from the SSOMV. Lastly, the sequence of the target gene that is identical to the homologous regions may be adjacent in the target gene but separated by one, two, or more nucleotides in the sequence of the SSOMV. Such an SSOMV causes an insertion in the sequence of the target gene. In certain embodiments, a SSOMV does not anneal to itself.

The nucleotides of the SSOMV are deoxyribonucleotides that are linked by unmodified phosphodiester bonds except that the 3′ terminal and/or 5′ terminal internucleotide linkage or alternatively the two 3′ terminal and/or 5′ terminal internucleotide linkages can be a phosphorothioate or phosphoamidate. As used herein an internucleotide linkage is the linkage between nucleotides of the SSOMV and does not include the linkage between the 3′ end nucleotide or 5′ end nucleotide and a blocking substituent. In a specific embodiment the length of the SSOMV is between 21 and 55 deoxynucleotides and the lengths of the homology regions are, accordingly, a total length of at least 20 deoxynucleotides and at least two homology regions should each have lengths of at least 8 deoxynucleotides.

The SSOMV can be designed to be complementary to either the coding or the non-coding strand of the target gene. When the desired mutation is a substitution of a single base, it is preferred that both the mutator nucleotide and the targeted nucleotide be a pyrimidine. To the extent that is consistent with achieving the desired functional result, it is preferred that both the mutator nucleotide and the targeted nucleotide in the complementary strand be pyrimidines. Particularly preferred are SSOMVs that encode transversion mutations, i.e., a C or T mutator nucleotide is mismatched, respectively, with a C or T nucleotide in the complementary strand.

2′-OME GRON Design. In various embodiments, a GRON may have both RNA and DNA nucleotides and/or other types of nucleobases. In some embodiments, one or more of the DNA or RNA nucleotides comprise a modification. In certain embodiments, the first 5′ nucleotide is an RNA nucleotide, and the remainder of the nucleotides are DNA. In still further embodiments, the first 5′ RNA nucleotide is modified with a 2-O-Me. In other embodiments, the first two, three, four, five, six, seven, eight, nine, ten or more 5′ nucleotides are an RNA nucleotide, and the remainder of the nucleotides are DNA. In still further embodiments, one or more of the first two, three, four, five, six, seven, eight, nine, ten or more 5′ RNA nucleotide is modified with a 2′-O-Me. In plant cells, double-strand beaks in DNA are typically repaired by the NHEJ DNA repair pathway. This pathway does not require a template to repair the DNA and is therefore error prone. The advantage of using this pathway to repair DNA for a plant cell is that it is quick, ubiquitous and most importantly can occur at times when a cell is not undergoing DNA replication. Another DNA repair pathway that functions in repairing double-strand breaks outside of the replication fork in plant cells is called templated repair; however, unlike the NHEJ pathway this type of repair is precise and requires the use of a DNA template (GRON).

### Improving Efficiency

The present disclosure may include any of a number of approaches to increase the effectiveness of conversion of a target gene using repair oligonucleotides, and which may be used alone or in combination with one another. These include, for example:
1. Introducing modifications to the repair oligonucleotides which attract DNA repair machinery to the targeted (mismatch) site.
   A. Introduction of one or more abasic sites in the oligonucleotide (e.g., within 10 bases, and in some embodiments with 5 bases of the desired mismatch site) generates a lesion which is an intermediate in base excision repair (BER), and which attracts BER machinery to the vicinity of the site targeted for conversion by the repair oligonucleotide. dSpacer (abasic furan) modified oligonucleotides may be prepared as described in, for example, Takeshita et al., J. Biol. Chem., 262:10171-79, 1987.
   B. Inclusion of compounds which induce single or double strand breaks, either into the oligonucleotide or together with the oligonucleotide, generates a lesion which is repaired by NHEJ, microhomology-mediated end joining (MMEJ), and homologous recombination. By way of example, the bleomycin family of antibiotics, zinc fingers, FokI (or any type IIS class of restriction enzyme) and other nucleases may be covalently coupled to the 3′ or 5′ end of repair oligonucleotides, in order to introduce double strand breaks in the vicinity of the site targeted for conversion by the repair oligonucleotide. The term “vicinity” in this context indicates that the single or double strand breaks are introduced within 2 kb, and preferably within 1 kb or less, of the amino acid residue intended to be converted (e.g., substituted, inserted, or deleted). The bleomycin family of antibiotics are DNA cleaving glycopeptides which include bleomycin, zeocin, phleomycin, tallysomycin, pepleomycin and others.
   C. Introduction of one or more 8′oxo dA or dG incorporated in the oligonucleotide (e.g., within 10 bases, and in some embodiments with 5 bases of the desired mismatch site) generates a lesion which is similar to lesions created by reactive oxygen species. These lesions induce the so-called “pushing repair” system. See, e.g., Kim et al., J. Biochem. Mol. Biol. 37:657-62, 2004.
2. Increase stability of the repair oligonucleotides:
   Introduction of a reverse base (idC) at the 3′ end of the oligonucleotide to create a 3′ blocked end on the repair oligonucleotide.
   Introduction of one or more 2′O-methyl nucleotides or bases which increase hybridization energy (see, e.g., WO2007/073149) at the 5′ and/or 3′ of the repair oligonucleotide.
   Introduction of one or a plurality of 2′O-methyl RNA nucleotides at the 5′ end of the repair oligonucleotide, leading into DNA bases which provide the desired mismatch site, thereby creating an Okazaki Fragment-like nucleic acid structure.
   Conjugated (5′ or 3′) intercalating dyes such as acridine, psoralen, ethidium bromide and Syber stains.
   Introduction of a 5′ terminus cap such as a T/A clamp, a cholesterol moiety, SIMA (HEX), riboC and amidite.
   Backbone modifications such as phosphothioate, 2′-O methyl, methyl phosphonates, locked nucleic acid (LNA), MOE (methoxyethyl), diPS and peptide nucleic acid (PNA).
   Crosslinking of the repair oligonucleotide, e.g., with intrastrand crosslinking reagents agents such as cisplatin and mitomycin C.
   Conjugation with fluorescent dyes such as Cy3, DY547, Cy3.5, Cy3B, Cy5 and DY647.
3. Increase hybridization energy of the repair oligonucleotide through incorporation of bases which increase hybridization energy (see, e.g., WO2007/073149).
4. Increase the quality of repair oligonucleotide synthesis by using nucleotide multimers (dimers, trimers, tetramers, etc.) as building blocks for synthesis. This results in fewer coupling steps and easier separation of the full-length products from building blocks.
5. Use of long repair oligonucleotides (i.e., greater than 55 nucleotides in length, for example such as the lengths described herein, for example having one or more mutations or two or more mutations targeted in the repair oligonucleotide.

Examples of the foregoing approaches are provided in the following table:

### Exemplary GRON Chemistries.

| | Oligo type | Modifications |
|---|---|---|
| 5′ mods | T/A clamp | T/A clamp |
| Backbone | Phosphothioate | PS |
| modifications | | |
| Intercalating dyes | 5′ Acridine 3′ | idC Acridine, idC |
| 2′-O-methyl | | DNA/RNA |
| Cy3 replacements | | DY547 |
| Facilitators | 2′-O-Me oligos designed 5′ | 2′-O-Me |
| | and 3′ of the converting oligo | |
| Abasic | Abasic site placed in various | Abasic 2 |
| | locations 5′ and 3′ to the | |
| | converting base. 44 mer | |
| Assist | Assist approach | Cy3, idC on one, |
| | Overlap: | none on the other: |
| | 2 oligos: 1 with Cy3/idC, | |
| | 1 unmodified repair oligo | |
| Assist | Assist approach | only make the |
| | No overlap: | unmodified oligo |
| | 2 oligos: 1 with Cy3/idC, 1 | |
| | unmodified repair oligo | |
| Abasic | THF site placed in various | Tetrahydrofuran |
| | locations 5′ and 3′ to the | (dspacer) |
| | converting base. 44 mer | |
| Backbone | 9 | 2′-O-Me |
| modifications | | |
| Trimers | | Trimer amidites, Cy3. |
| | | idC |
| Pushing repair | | 8′oxo dA, 5′ Cy3, idC |
| Pushing repair | | 8′oxo dA, 5′ Cy3, idC |
| Double Strand | | Bleomycin |
| Break | | |
| Crosslinker | | Cisplatin |
| Crosslinker | | Mitomycin C |
| Facilitators | super bases 5′ and 3′ of | 2 amino dA and 2- |
| | converting oligo | thio T |
| Super oligos | | 2′amino d, 5′ Cy3, idC |
| Super oligos | | 2-thio T, 5′ Cy3, idC |
| Super oligos | | 7-deaza A, 5′ Cy3, idC |
| Super oligos | | 7-deaza G, 5′ Cy3, idC |
| Super oligos | | propanyl dC, 5′ Cy3, |
| | | idC |
| Intercalating dyes | 5′ Psoralen/3′ idC | Psoralen, idC |
| Intercalating dyes | 5′ Ethidium bromide | Ethidium bromide |
| Intercalating dyes | 5′ Syber stains | Syber stains |
| 5′ mods | 5′ Chol/3′ idC | Cholesterol |
| Double mutation | Long oligo (55+ bases) w/ | Any modification |
| | 2 mutation | |
| 5′ mods | 5′ SIMA HEX/3′idC | SIMA HEX, idC |
| Backbone | 9 | Methyl phosphonates |
| modifications | | |
| Backbone | | LNA |
| modifications | | |
| Backbone | 1 | MOE (methoxyethyl) |
| modifications | | |
| Cy3 replacements | | Cy3.5 |
| Cy3 replacements | | Cy5 |
| Backbone | | diPS |
| modifications | | |
| 5′ mods | | riboC for branch nm |
| Backbone | | PNA |
| modifications | | |
| Cy3 replacements | | DY647 |
| 5′ mods | 5′ branch | symmetric branch |
| | | amidite/idC |

The foregoing modifications may also include known nucleotide modifications such as methylation, 5′ intercalating dyes, modifications to the 5′ and 3′ ends, backbone modifications, crosslinkers, cyclization and ‘caps’ and substitution of one or more of the naturally occurring nucleotides with an analog such as inosine. Modifications of nucleotides include the addition of acridine, amine, biotin, cascade blue, cholesterol, Cy3®, Cy5®, Cy5.5® Daboyl, digoxigenin, dinitrophenyl, Edans, 6-FAM, fluorescein, 3′-glyceryl, HEX, IRD-700, IRD-800, JOE, phosphate psoralen, rhodamine, ROX, thiol (SH), spacers, TAMRA, TET, AMCA-S″, SE, BODIPY, Marina Blue®, Pacific Blue®, Oregon Green®, Rhodamine Green®, Rhodamine Red®, Rhodol Green® and Texas Red®. Polynucleotide backbone modifications include methylphosphonate, 2′-OMe-methylphosphonate RNA, phosphorothiorate, RNA, 2′-OMeRNA. Base modifications include 2-amino-dA, 2-aminopurine, 3′-(ddA), 3′dA (cordycepin), 7-deaza-dA, 8-Br-dA, 8-oxo-dA, N6-Me-dA, abasic site (dSpacer), biotin dT, 2′-OMe-5Me-C, 2′-OMe-propynyl-C, 3′-(5-Me-dC), 3′-(ddC), 5-Br-dC, 5-1-duc, 5-Me-dC, 5-F-dC, carboxy-dT, convertible dA, convertible dC, convertible dG, convertible dT, convertible dU, 7-deaza-dG, 8-Br-dG, 8-oxo-dG, 06-Me-dG, S6-DNP-dG, 4-methyl-indole, 5-nitroindole, 2′-OMe-inosine, 2′-dl, 06-phenyl-dl, 4-methyl-indole, 2′-deoxynebularine, 5-nitroindole, 2-aminopurine, dP (purine analogue), dK (pyrimidine analogue), 3-nitropyrrole, 2-thio-dT, 4-thio-dT, biotin-dT, carboxy-dT, 04-Me-dT, 04-triazol dT, 2′-OMe-propynyl-U, 5-Br-dU, 2′-dU, 5-F-dU, 5-1-dU, 04-triazol dU. Said terms also encompass peptide nucleic acids (PNAs), a DNA analogue in which the backbone is a pseudopeptide consisting of N-(2-aminoethyl)-glycine units rather than a sugar. PNAs mimic the behavior of DNA and bind complementary nucleic acid strands. The neutral backbone of PNA results in stronger binding and greater specificity than normally achieved. In addition, the unique chemical, physical and biological properties of PNA have been exploited to produce powerful biomolecular tools, antisense and antigene agents, molecular probes and biosensors.

Oligonucleobases may have nick(s), gap(s), modified nucleotides such as modified oligonucleotide backbones, abasic nucleotides, or other chemical moieties. In a further embodiment, at least one strand of the oligonucleobase includes at least one additional modified nucleotide, e.g., a 2′-O-methyl modified nucleotide such as a MOE (methoxyethyl), a nucleotide having a 5′-phosphorothioate group, a terminal nucleotide linked to a cholesteryl derivative, a 2′-deoxy-2′-fluoro modified nucleotide, a 2′-deoxy-modified nucleotide, a locked nucleotide, an abasic nucleotide (the nucleobase is missing or has a hydroxyl group in place thereof (see, e.g., Glen Research, http://www.glenresearch.com/GlenReports/GR21-14.html—(acquired by Maravai LifeSciences))), a 2′-amino-modified nucleotide, a 2′-alkyl-modified nucleotide, a morpholino nucleotide, a phosphoramidite, and a non-natural base comprising nucleotide. Various salts, mixed salts and free acid forms are also included.

Preferred modified oligonucleotide backbones include, for example, phosphorothioates, chiral phosphorothioates, phosphoro-dithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e., a single inverted nucleoside residue which may be abasic (the nucleobase is missing or has a hydroxyl group in place thereof). The most common use of a linkage inversion is to add a 3′-3′ linkage to the end of an antisense oligonucleotide with a phosphorothioate backbone. The 3′-3′ linkage further stabilizes the antisense oligonucleotide to exonuclease degradation by creating an oligonucleotide with two 5′-OH ends and no 3′-OH end. Linkage inversions can be introduced into specific locations during oligonucleotide synthesis through use of “reversed phosphoramidites”. These reagents have the phosphoramidite groups on the 5′-OH position and the dimethoxytrityl (DMT) protecting group on the 3′-OH position. Normally, the DMT protecting group is on the 5′-OH and the phosphoramidite is on the 3′-OH.

Examples of modified bases include, but are not limited to, 2-aminopurine, 2′-amino-butyryl pyrene-uridine, 2′-aminouridine, 2′-deoxyuridine, 2′-fluoro-cytidine, 2′-fluoro-uridine, 2,6-diaminopurine, 4-thio-uridine, 5-bromo-uridine, 5-fluoro-cytidine, 5-fluorouridine, 5-indo-uridine, 5-methyl-cytidine, inosine, N3-methyl-uridine, 7-deaza-guanine, 8-aminohexyl-amino-adenine, 6-thio-guanine, 4-thio-thymine, 2-thio-thymine, 5-iodo-uridine, 5-iodo-cytidine, 8-bromo-guanine, 8-bromo-adenine, 7-deaza-adenine, 7-diaza-guanine, 8-oxo-guanine, 5,6-dihydro-uridine, and 5-hydroxymethyl-uridine. These synthetic units are commercially available; (for example, purchased from Glen Research Company-(acquired by Maravai LifeSciences)) and can be incorporated into DNA by chemical synthesis.

Examples of modification of the sugar moiety are 3′-deoxylation, 2′-fluorination, and arabanosidation, however, it is not to be construed as being limited thereto. Incorporation of these into DNA is also possible by chemical synthesis.

Examples of the 5′ end modification are 5′-amination, 5′-biotinylation, 5′-fluoresceinylation, 5′-tetrafluoro-fluoreceinyaltion, 5′-thionation, and 5′-dabsylation, however it is not to be construed as being limited thereto.

Examples of the 3′ end modification are 3′-amination, 3′-biotinylation, 2,3-dideoxidation, 3′-thionation, 3′-dabsylation, 3′-carboxylation, and 3′-cholesterylation, however, it is not to be construed as being limited thereto.

In one preferred embodiment, the oligonucleobase can contain a 5′ blocking substituent that is attached to the 5′ terminal carbons through a linker. The chemistry of the linker is not critical other than its length, which should in some embodiments be at least 6 atoms long and that the linker should be flexible. A variety of non-toxic substituents such as biotin, cholesterol or other steroids or a non-intercalating cationic fluorescent dye can be used. Particularly preferred reagents to make oligonucleobases are the reagents sold as Cy3 and Cy5 by Glen Research, Sterling Va. (acquired by Maravai LifeSciences), which are blocked phosphoroamidites that upon incorporation into an oligonucleotide yield 3,3,3′,3′-tetramethyl N,N′-isopropyl substituted indomonocarbocyanine and indodicarbocyanine dyes, respectively. Cy3 is particularly preferred. When the indocarbocyanine is N-oxyalkyl substituted it can be conveniently linked to the 5′ terminal of the oligodeoxynucleotide as a phosphodiester with a 5′ terminal phosphate. When the commercially available Cy3 phosphoramidite is used as directed, the resulting 5′ modification consists of a blocking substituent and linker together which are a N-hydroxypropyl, N′-phosphatidylpropyl 3,3,3′,3′-tetramethyl indomonocarbocyanine. Other dyes contemplated include Rhodamine6G, Tetramethylrhodamine, Sulforhodamine 101, Merocyanine 540, Atto565, Atto550 26, Cy3.5, Dy547, Dy548, Dy549, Dy554, Dy555, Dy556, Dy560, mStrawberry and mCherry.

In a preferred embodiment the indocarbocyanine dye is tetra substituted at the 3 and 3′ positions of the indole rings. Without limitations as to theory these substitutions prevent the dye from being an intercalating dye. The identity of the substituents at these positions is not critical.

The oligo designs herein described might also be used as more efficient donor templates in combination with other DNA editing or recombination technologies including, but not limited to, gene targeting using site-specific homologous recombination by zinc finger nucleases, meganucleases, Transcription Activator-Like Effector Nucleases (TALENs) or Clustered Regularly Interspaced Short Palindromic Repeats (CRISPRs).

The present disclosure in certain aspects and embodiments may include methods and compositions relating to methods for the efficient modification of genomic cellular DNA and/or recombination of DNA into the genomic DNA of cells. Although not limited to any particular use, some methods provided herein may in certain embodiments be useful in, for example, introducing a modification into the genome of a cell for the purpose of determining the effect of the modification on the cell. For example, a modification may be introduced into the nucleotide sequence which encodes an enzyme to determine whether the modification alters the enzymatic activity of the enzyme, and/or determine the location of the enzyme's catalytic region. Alternatively, the modification may be introduced into the coding sequence of a DNA-binding protein to determine whether the DNA binding activity of the protein is altered, and thus to delineate the particular DNA-binding region within the protein. Yet another alternative is to introduce a modification into a non-coding regulatory sequence (e.g., promoter, enhancer, regulatory RNA sequence (miRNA), etc.) in order to determine the effect of the modification on the level of expression of a second sequence which is operably linked to the non-coding regulatory sequence. This may be desirable to, for example, define the particular sequence which possesses regulatory activity.

### DNA Cutters

One strategy for producing targeted gene disruption is through the generation of single strand or double strand DNA breaks using a DNA cutter such as a site-specific endonuclease. Endonucleases are most often used for targeted gene disruption in organisms that have traditionally been refractive to more conventional gene targeting methods, such as algae, plants, and large animal models, including humans. For example, there are currently human clinical trials underway involving zinc finger nucleases for the treatment and prevention of HIV infection. Additionally, endonuclease engineering is currently being used in attempts to disrupt genes that produce undesirable phenotypes in crops.

Certain aspects of the present disclosure related to introducing one or more mutations into a targeted nucleic acid using a DNA endonuclease. In some embodiments, the DNA endonuclease is an RNA-guided DNA endonuclease. Exemplary RNA-guided DNA endonucleases include but are not limited to Cas9, Cas12, Cpf1, MADzymes and their variants, analogues, homologues, and orthologues. In some embodiments, the DNA endonuclease is a MADzyme including but not limited to MAD7, a codon-optimized endonuclease, released by Inscripta, Inc. See U.S. Pat. Nos. 9,982,279 and 10,435,714; each of which is hereby incorporated by reference in its entirety. RNA-guided DNA endonucleases suitable for use in the methods and compositions described herein will be readily apparent to one of skill in the art. Additional DNA endonucleases for use in the methods and compositions of the present disclosure are described herein.

### Zinc Fingers

One class of artificial endonucleases is the zinc finger endonucleases. Zinc finger endonucleases combine a non-specific cleavage domain, typically that of FokI endonuclease, with zinc finger protein domains that are engineered to bind to specific DNA sequences. The modular structure of the zinc finger endonucleases makes them a versatile platform for delivering site-specific double-strand breaks to the genome. As FokI endonuclease cleaves as a dimer, one strategy to prevent off-target cleavage events has been to design zinc finger domains that bind at adjacent 9 base pair sites. See also U.S. Pat. Nos. 7,285,416; 7,521,241; 7,361,635; 7,273,923; 7,262,054; 7,220,719; 7,070,934; 7,013,219; 6,979,539; 6,933,113; 6,824,978; each of which is hereby herein incorporated by reference in its entirety.

### TALENs

TALENs are targetable nucleases are used to induce single- and double-strand breaks into specific DNA sites, which are then repaired by mechanisms that can be exploited to create sequence alterations at the cleavage site.

The fundamental building block that is used to engineer the DNA-binding region of TALENs is a highly conserved repeat domain derived from naturally occurring TALEs encoded by Xanthomonas spp. proteobacteria. DNA binding by a TALEN is mediated by arrays of highly conserved 33-35 amino acid repeats that are flanked by additional TALE-derived domains at the amino-terminal and carboxy-terminal ends of the repeats.

These TALE repeats specifically bind to a single base of DNA, the identity of which is determined by two hypervariable residues typically found at positions 12 and 13 of the repeat, with the number of repeats in an array corresponded to the length of the desired target nucleic acid, the identity of the repeat selected to match the target nucleic acid sequence. In some embodiments, the target nucleic acid is between 15 and 20 base pairs in order to maximize selectivity of the target site. Cleavage of the target nucleic acid typically occurs within 50 base pairs of TALEN binding. Computer programs for TALEN recognition site design have been described in the art. See, e.g., Cermak et al., Nucleic Acids Res. 2011 July; 39 (12): e82.

Once designed to match the desired target sequence, TALENs can be expressed recombinantly and introduced into protoplasts as exogenous proteins or expressed from a plasmid within the protoplast or administered as mRNA or as protein.

### Meganucleases

The homing endonucleases, also known as meganucleases, are sequence specific endonucleases that generate double strand breaks in genomic DNA with a high degree of specificity due to their large (e.g., >14 bp) cleavage sites. While the specificity of the homing endonucleases for their target sites allows for precise targeting of the induced DNA breaks, homing endonuclease cleavage sites are rare and the probability of finding a naturally occurring cleavage site in a targeted gene is low.

Another class of artificial endonucleases is the engineered meganucleases. Engineered homing endonucleases are generated by modifying the specificity of existing homing endonucleases. In one approach, variations are introduced in the amino acid sequence of naturally occurring homing endonucleases and then the resultant engineered homing endonucleases are screened to select functional proteins which cleave a targeted binding site. In another approach, chimeric homing endonucleases are engineered by combining the recognition sites of two different homing endonucleases to create a new recognition site composed of a half-site of each homing endonuclease. See e.g., U.S. Pat. Nos. 8,338,157, and 8,445,251.

### CRISPRs or CRISPR/Cas Systems

CRISPR-Cas system contains three basic design components: 1) Cas gene, transcript (e.g., mRNA) or protein; 2) guide RNA (gRNA); and 3) crRNAs (CRISPR RNA) are RNA segments processed from RNA transcripts encoding the CRISPR repeat arrays, which harbor a “protospacer” region that are complementary to a foreign DNA site (e.g., endogenous DNA target region) and a part of the CRISPR repeat. See e.g., PCT Application Nos WO/2014/093661 and WO/2013/176772.

Cas (CRISPR Associated) Gene, Transcript (e.g., mRNA) or Protein

Transient Cas expression from a plasmid vector, direct delivery of Cas protein and or direct delivery of Cas mRNA into plant cells. Cas genes are codon optimized for expression in higher plants, algae or yeast and are driven by either a constitutive, inducible, tissue-specific or species-specific promoter when applicable. Cas transcript termination and polyadenlyation signals are either NosT, RBCT, HSP18.2T or other gene specific or species-specific terminators. Cas gene cassettes may contain introns, either native or in combination with gene-specific promoters and or synthetic promoters. Cas protein may contain one or more nuclear localization signal sequences (NLS), mutations, deletions, alterations or truncations. In transient expression systems, Cas gene cassettes may be combined with other components of the CRISPR-Cas system such as gRNA cassettes on the same transient expression vector. Alternatively, Cas gene cassettes may be located and expressed from constructs independent of gRNA cassettes or from other components of the CRISPR-Cas system. CRISPR associated (Cas) gene—encode for proteins with a variety of predicted nucleic acid-manipulating activities such as nucleases, helicases and polymerase. Cas genes include Cas9. Cas9 is a gene encoding a large protein containing a predicted RuvC-like and HNH endonuclease domains and is associated with the CRISPR adaptive immunity system that is present in most archaea and many bacteria. Cas9 protein consists of two lobes:
1) Recognition (REC) lobe—consists of three domains:
   a) BH (bridge helix)
   b) REC1—facilitates RNA-guided DNA targeting
   c) REC2—facilitates RNA-guided DNA targeting
2) Nuclease (NUC) lobe—consists of three domains:
   a) RuvC—facilitates RNA-guided DNA targeting; endonuclease activity
   b) HNH—endonuclease activity
   c) PI—PAM interacting

In other embodiments, the Cas gene may be a homolog of Cas9 in which the RuvC, HNH, REC and BH domains are highly conserved. In some embodiments, Cas genes are those from the following species listed in the following table:

### Exemplary Cas Genes

| Locus ID/GI | Species | Cas profile ID | Cas gene |
|---|---|---|---|
| 352684361 | Acidaminococcus_intestini_RyC_MR95_uid74445 | mkCas0193 | cas9 |
| 117929158 | Acidothermus_cellulolyticus_11B_uid58501 | cd09643 | cas9 |
| 326315085 | Acidovorax_avenae_ATCC_19860_uid42497 | cd09643 | cas9 |
| 222109285 | Acidovorax_ebreus_TPSY_uid59233 | COG3513 | cas9 |
| 152978060 | Actinobacillus_succinogenes_130Z_uid58247 | COG3513 | cas9 |
| 407692091 | Actinobacillus_suis_H91_0380_uid176363 | COG3513 | cas9 |
| 187736489 | Akkermansia_muciniphila_ATCC_BAA_835_uid58985 | cd09643 | cas9 |
| 319760940 | Alicycliphilus_denitrificans_BC_uid49953 | cd09643 | cas9 |
| 330822845 | Alicycliphilus_denitrificans_K601_uid66307 | cd09643 | cas9 |
| 288957741 | Azospirillum_B510_uid46085 | cd09643 | cas9 |
| 549484339 | Bacteroides_CF50_uid222805 | cd09643, COG3513 | cas9 |
| 375360193 | Bacteroides_fragilis_638R_uid84217 | COG3513, COG3513 | cas9 |
| 60683389 | Bacteroides_fragilis_NCTC_9343_uid57639 | COG3513, COG3513 | cas9 |
| 471261880 | Bdellovibrio_exovorus_JSS_uid194119 | COG3513 | cas9 |
| 390944707 | Belliella_baltica_DSM_15883_uid168182 | cd09643, COG3513 | cas9 |
| 470166767 | Bibersteinia_trehalosi_192_uid193709 | COG3513 | cas9 |
| 310286728 | Bifidobacterium_bifidum_S17_uid59545 | mkCas0193 | cas9 |
| 283456135 | Bifidobacterium_dentium_Bd1_uid43091 | cd09643 | cas9 |
| 189440764 | Bifidobacterium_longum_DJO10A_uid58833 | cd09643 | cas9 |
| 384200944 | Bifidobacterium_longum_KACC_91563_uid158861 | cd09643 | cas9 |
| 479188345 | Butyrivibrio_fibrisolvens_uid197155 | cd09643 | cas9 |
| 544063172 | Campylobacter_jejuni_00_2425_uid219359 | COG3513 | cas9 |
| 543948719 | Campylobacter_jejuni_00_2426_uid219324 | COG3513 | cas9 |
| 543946932 | Campylobacter_jejuni_00_2538_uid219325 | COG3513 | cas9 |
| 543950499 | Campylobacter_jejuni_00_2544_uid219326 | COG3513 | cas9 |
| 549693479 | Campylobacter_jejuni_4031_uid222817 | COG3513 | cas9 |
| 157415744 | Campylobacter_jejuni_81116_uid58771 | COG3513 | cas9 |
| 384448746 | Campylobacter_jejuni_IA3902_uid159531 | COG3513 | cas9 |
| 384442102 | Campylobacter_jejuni_M1_uid159535 | COG3513 | cas9 |
| 384442103 | Campylobacter_jejuni_M1_uid159535 | COG3513 | cas9 |
| 403056243 | Campylobacter_jejuni_NCTC_11168_BN148_uid174152 | COG3513 | cas9 |
| 218563121 | Campylobacter_jejuni_NCTC_11168_ATCC_700819_uid57587 | COG3513 | cas9 |
| 407942868 | Campylobacter_jejuni_PT14_uid176499 | COG3513 | cas9 |
| 153952471 | Campylobacter_jejuni_doylei_269_97_uid58671 | COG3513 | cas9 |
| 294086111 | Candidatus_Puniceispirillum_marinum_IMCC1322_uid47081 | cd09643 | cas9 |
| 340622236 | Capnocytophaga_canimorsus_Cc5_uid70727 | COG3513, cd09643 | cas9 |
| 220930482 | Clostridium_cellulolyticum_H10_uid58709 | COG3513 | cas9 |
| 479136975 | Coprococcus_catus_GD_7_uid197174 | mkCas0193 | cas9 |
| 328956315 | Coriobacterium_glomerans_PW2_uid65787 | mkCas0193 | cas9 |
| 375289763 | Corynebacterium_diphtheriae_241_uid83607 | cd09643 | cas9 |
| 376283539 | Corynebacterium_diphtheriae_31A_uid84309 | cd09643 | cas9 |
| 376286566 | Corynebacterium_diphtheriae_BH8_uid84311 | cd09643 | cas9 |
| 376289243 | Corynebacterium_diphtheriae_C7_beta_uid84313 | cd09643 | cas9 |
| 376244596 | Corynebacterium_diphtheriae_HC01_uid84297 | cd09643 | cas9 |
| 376292154 | Corynebacterium_diphtheriae_HC02_uid84317 | cd09643 | cas9 |
| 38232678 | Corynebacterium_diphtheriae_NCTC_13129_uid57691 | cd09643 | cas9 |
| 376256051 | Corynebacterium_diphtheriae_VA01_uid84305 | cd09643 | cas9 |
| 159042956 | Dinoroseobacter_shibae_DFL_12_uid58707 | cd09643 | cas9 |
| 339445983 | Eggerthella_YY7918_uid68707 | mkCas0193 | cas9 |
| 187250660 | Elusimicrobium_minutum_Pei191_uid58949 | cd09643 | cas9 |
| 479180325 | Enterococcus_7L76_uid197170 | cd09643 | cas9 |
| 397699066 | Enterococcus_faecalis_D32_uid171261 | mkCas0193 | cas9 |
| 384512368 | Enterococcus_faecalis_OG1RF_uid54927 | mkCas0193 | cas9 |
| 392988474 | Enterococcus_hirae_ATCC_9790_uid70619 | mkCas0193 | cas9 |
| 558685081 | Enterococcus_mundtii_QU_25_uid229420 | mkCas0193 | cas9 |
| 238924075 | Eubacterium_rectale_ATCC_33656_uid59169 | cd09643 | cas9 |
| 385789535 | Fibrobacter_succinogenes_S85_uid161919 | cd09643, cd09643 | cas9 |
| 261414553 | Fibrobacter_succinogenes_S85_uid41169 | cd09643, cd09643 | cas9 |
| 374307738 | Filifactor_alocis_ATCC_35896_uid46625 | mkCas0193 | cas9 |
| 169823755 | Finegoldia_magna_ATCC_29328_uid58867 | mkCas0193 | cas9 |
| 150025575 | Flavobacterium_psychrophilum_JIP02_86_uid61627 | cd09643, cd09643 | cas9 |
| 327405121 | Fluviicola_taffensis_DSM_16823_uid65271 | cd09643, cd09643 | cas9 |
| 387824704 | Francisella_cf_novicida_3523_uid162107 | cd09704 | cas9 |
| 118497352 | Francisella_novicida_U112_uid58499 | cd09704 | cas9 |
| 134302318 | Francisella_tularensis_WY96_3418_uid58811 | cd09704 | cas9 |
| 89256630 | Francisella_tularensis_holarctica_LVS_uid58595 | cd09704 | cas9 |
| 89256631 | Francisella_tularensis_holarctica_LVS_uid58595 | cd09704 | cas9 |
| 534508854 | Fusobacterium_3_1_36A2_uid55995 | mkCas0193 | cas9 |
| 530600688 | Geobacillus_JF8_uid215234 | COG3513 | cas9 |
| 209542524 | Gluconacetobacter_diazotrophicus_PAl_5_uid59075 | COG3513 | cas9 |
| 162147907 | Gluconacetobacter_diazotrophicus_PAl_5_uid61587 | COG3513 | cas9 |
| 479173968 | Gordonibacter_pamelaeae_7_10_1_b_uid197167 | mkCas0193 | cas9 |
| 345430422 | Haemophilus_parainfluenzae_T3T1_uid72801 | COG3513 | cas9 |
| 471315929 | Helicobacter_cinaedi_ATCC_BAA_847_uid193765 | COG3513 | cas9 |
| 386762035 | Helicobacter_cinaedi_PAGU611_uid162219 | COG3513 | cas9 |
| 291276265 | Helicobacter_mustelae_12198_uid46647 | COG3513 | cas9 |
| 385811609 | Ignavibacterium_album_JCM_16511_uid162097 | cd09643, COG3513 | cas9 |
| 310780384 | Ilyobacter_polytropus_DSM_2926_uid59769 | COG3513 | cas9 |
| 331702228 | Lactobacillus_buchneri_NRRL_B_30929_uid66205 | mkCas0193 | cas9 |
| 406027703 | Lactobacillus_buchneri_uid73657 | mkCas0193 | cas9 |
| 385824065 | Lactobacillus_casei_BD_II_uid162119 | mkCas0193 | cas9 |
| 191639137 | Lactobacillus_casei_BL23_uid59237 | mkCas0193 | cas9 |
| 385820880 | Lactobacillus_casei_LC2W_uid162121 | mkCas0193 | cas9 |
| 523514789 | Lactobacillus_casei_LOCK919_uid210959 | mkCas0193 | cas9 |
| 409997999 | Lactobacillus_casei_W56_uid178736 | mkCas0193 | cas9 |
| 301067199 | Lactobacillus_casei_Zhang_uid50673 | mkCas0193 | cas9 |
| 385815562 | Lactobacillus_delbrueckii_bulgaricus_2038_uid161929 | mkCas0193 | cas9 |
| 385815563 | Lactobacillus_delbrueckii_bulgaricus_2038_uid161929 | mkCas0193 | cas9 |
| 385815564 | Lactobacillus_delbrueckii_bulgaricus_2038_uid161929 | mkCas0193 | cas9 |
| 385826041 | Lactobacillus_johnsonii_DPC_6026_uid162057 | mkCas0193 | cas9 |
| 532357525 | Lactobacillus_paracasei_8700_2_uid55295 | mkCas0193 | cas9 |
| 448819853 | Lactobacillus_plantarum_ZJ316_uid188689 | mkCas0193 | cas9 |
| 385828839 | Lactobacillus_rhamnosus_GG_uid161983 | mkCas0193 | cas9 |
| 258509199 | Lactobacillus_rhamnosus_GG_uid59313 | mkCas0193 | cas9 |
| 523517690 | Lactobacillus_rhamnosus_LOCK900_uid210957 | mkCas0193 | cas9 |
| 385839898 | Lactobacillus_salivarius_CECT_5713_uid162005 | mkCas0193 | cas9 |
| 385839899 | Lactobacillus_salivarius_CECT_5713_uid162005 | mkCas0193 | cas9 |
| 385839900 | Lactobacillus_salivarius_CECT_5713_uid162005 | mkCas0193 | cas9 |
| 90961083 | Lactobacillus_salivarius_UCC118_uid58233 | mkCas0193 | cas9 |
| 90961084 | Lactobacillus_salivarius_UCC118_uid58233 | mkCas0193 | cas9 |
| 347534532 | Lactobacillus_sanfranciscensis_TMW_1_1304_uid72937 | mkCas0193 | cas9 |
| 54296138 | Legionella_pneumophila_Paris_uid58211 | cd09704 | cas9 |
| 406600271 | Leuconostoc_gelidum_JB7_uid175682 | mkCas0193 | cas9 |
| 16801805 | Listeria_innocua_Clip11262_uid61567 | cd09643, COG3513 | cas9 |
| 386044902 | Listeria_monocytogenes_10403S_uid54461 | COG3513, COG3513 | cas9 |
| 550898770 | Listeria_monocytogenes_EGD_uid223288 | COG3513, COG3513 | cas9 |
| 386048324 | Listeria_monocytogenes_J0161_uid54459 | COG3513, COG3513 | cas9 |
| 405756714 | Listeria_monocytogenes_SLCC2540_uid175106 | COG3513, COG3513 | cas9 |
| 404411844 | Listeria_monocytogenes_SLCC5850_uid175110 | COG3513, COG3513 | cas9 |
| 404282159 | Listeria_monocytogenes_serotype_1_2b_SLCC2755_uid52455 | COG3513, COG3513 | cas9 |
| 404287973 | Listeria_monocytogenes_serotype_7_SLCC2482_uid174871 | COG3513, COG3513 | cas9 |
| 433625054 | Mycoplasma_cynos_C142_uid184824 | cd09643 | cas9 |
| 401771107 | Mycoplasma_gallisepticum_CA06_2006_052_5_2P_uid172630 | cd09643 | cas9 |
| 385326554 | Mycoplasma_gallisepticum_F_uid162001 | cd09643 | cas9 |
| 401767318 | Mycoplasma_gallisepticum_NC95_13295_2_2P_uid172625 | cd09643 | cas9 |
| 401768090 | Mycoplasma_gallisepticum_NC96_1596_4_2P_uid172626 | cd09643 | cas9 |
| 401768851 | Mycoplasma_gallisepticum_NY01_2001_047_5_1P_uid172627 | cd09643 | cas9 |
| 385325798 | Mycoplasma_gallisepticum_R_high_uid161999 | cd09643 | cas9 |
| 294660600 | Mycoplasma_gallisepticum_R_low_uid57993 | cd09643 | cas9 |
| 565627373 | Mycoplasma_gallisepticum_S6_uid200523 | cd09643 | cas9 |
| 401769598 | Mycoplasma_gallisepticum_WI01_2001_043_13_2P_uid172628 | cd09643 | cas9 |
| 47458868 | Mycoplasma_mobile_163K_uid58077 | cd09643 | cas9 |
| 71894592 | Mycoplasma_synoviae_53_uid58061 | cd09643 | cas9 |
| 313669044 | Neisseria_lactamica_020_06_uid60851 | COG3513 | cas9 |
| 161869390 | Neisseria_meningitidis_053442_uid58587 | COG3513 | cas9 |
| 385324780 | Neisseria_meningitidis_8013_uid161967 | COG3513 | cas9 |
| 385337435 | Neisseria_meningitidis_WUE_2594_uid162093 | COG3513 | cas9 |
| 218767588 | Neisseria_meningitidis_Z2491_uid57819 | COG3513 | cas9 |
| 254804356 | Neisseria_meningitidis_alpha14_uid61649 | COG3513 | cas9 |
| 319957206 | Nitratifractor_salsuginis_DSM_16511_uid62183 | cd09643 | cas9 |
| 325983496 | Nitrosomonas_AL212_uid55727 | COG3513 | cas9 |
| 302336020 | Olsenella_uli_DSM_7084_uid51367 | mkCas0193 | cas9 |
| 392391493 | Ornithobacterium_rhinotracheale_DSM_15997_uid168256 | cd09643 | cas9 |
| 154250555 | Parvibaculum_lavamentivorans_DS_1_uid58739 | cd09643 | cas9 |
| 15602992 | Pasteurella_multocida_Pm70_uid57627 | COG3513 | cas9 |
| 557607382 | Pediococcus_pentosaceus_SL4_uid227215 | mkCas0193 | cas9 |
| 294674019 | Prevotella_ruminicola_23_uid47507 | COG3513 | cas9 |
| 408489713 | Psychroflexus_torquis_ATCC_700755_uid54205 | cd09643, cd09643 | cas9 |
| 90425961 | Rhodopseudomonas_palustris_BisB18_uid58443 | COG3513 | cas9 |
| 91975509 | Rhodopseudomonas_palustris_BisB5_uid58441 | COG3513 | cas9 |
| 83591793 | Rhodospirillum_rubrum_ATCC_11170_uid57655 | cd09643 | cas9 |
| 386348484 | Rhodospirillum_rubrum_F11_uidl62149 | cd09643 | cas9 |
| 383485594 | Riemerella_anatipestifer_ATCC_11845_DSM_15868_uid159857 | COG3513, cd09643 | cas9 |
| 407451859 | Riemerella_anatipestifer_RA_CH_1_uid175469 | COG3513, cd09643 | cas9 |
| 442314523 | Riemerella_anatipestifer_RA_CH_2_uid186548 | COG3513, cd09643 | cas9 |
| 386321727 | Riemerella_anatipestifer_RA_GD_uid162013 | COG3513, cd09643 | cas9 |
| 479204792 | Roseburia_intestinalis_uid197164 | COG3513 | cas9 |
| 470213512 | Sphingomonas_MM_1_uid193771 | COG3513 | cas9 |
| 325972003 | Spirochaeta_Buddy_uid63633 | cd09643 | cas9 |
| 563693590 | Spiroplasma_apis_B31_uid230613 | cd09643 | cas9 |
| 507384108 | Spiroplasma_syrphidicola_EA_1_uid205054 | cd09643 | cas9 |
| 556591142 | Staphylococcus_pasteuri_SP1_uid226267 | cd09643 | cas9 |
| 386318630 | Staphylococcus_pseudintermedius_ED99_uid162109 | mkCas0193 | cas9 |
| 269123826 | Streptobacillus_moniliformis_DSM_12112_uid41863 | COG3513 | cas9 |
| 552737657 | Streptococcus_I_G2_uid224251 | cd09643 | cas9 |
| 512539130 | Streptococcus_agalactiae_09mas018883_uid208674 | mkCas0193 | cas9 |
| 22537057 | Streptococcus_agalactiae_2603V_R_uid57943 | mkCas0193 | cas9 |
| 494703075 | Streptococcus_agalactiae_2_22_uid202215 | mkCas0193 | cas9 |
| 76788458 | Streptococcus_agalactiae_A909_uid57935 | mkCas0193 | cas9 |
| 406709383 | Streptococcus_agalactiae_GD201008_001_uid175780 | mkCas0193 | cas9 |
| 512544670 | Streptococcus_agalactiae_ILRI005_uid208676 | mkCas0193 | cas9 |
| 512698372 | Streptococcus_agalactiae_ILRI112_uid208675 | mkCas0193 | cas9 |
| 25010965 | Streptococcus_agalactiae_NEM316_uid61585 | mkCas0193 | cas9 |
| 410594450 | Streptococcus_agalactiae_SA20_06_uid178722 | mkCas0193 | cas9 |
| 538370328 | Streptococcus_anginosus_C1051_uid218003 | cd09643 | cas9 |
| 410494913 | Streptococcus_dysgalactiae_equisimilis_AC_2713_uid178644 | COG3513 | cas9 |
| 386317166 | Streptococcus_dysgalactiae_equisimilis_ATCC_12394_uid161979 | COG3513 | cas9 |
| 251782637 | Streptococcus_dysgalactiae_equisimilis_GGS_124_uid59103 | COG3513 | cas9 |
| 408401787 | Streptococcus_dysgalactiae_equisimilis_RE378_uid176684 | COG3513 | cas9 |
| 195978435 | Streptococcus_equi_zooepidemicus_MGCS10565_uid59263 | COG3513 | cas9 |
| 386338081 | Streptococcus_gallolyticus_ATCC_43143_uid162103 | cd09643 | cas9 |
| 386338091 | Streptococcus_gallolyticus_ATCC_43143_uid162103 | mkCas0193 | cas9 |
| 325978669 | Streptococcus_gallolyticus_ATCC_BAA_2069_uid63617 | mkCas0193 | cas9 |
| 288905632 | Streptococcus_gallolyticus_UCN34_uid46061 | cd09643 | cas9 |
| 288905639 | Streptococcus_gallolyticus_UCN34_uid46061 | mkCas0193 | cas9 |
| 157150687 | Streptococcus_gordonii_Challis_substr_CH1_uid57667 | cd09643 | cas9 |
| 379705580 | Streptococcus_infantarius_CJ18_uid87033 | mkCas0193 | cas9 |
| 508127396 | Streptococcus_iniae_SF1_uid206041 | mkCas0193 | cas9 |
| 508127399 | Streptococcus_iniae_SF1_uid206041 | COG3513 | cas9 |
| 538379999 | Streptococcus_intermedius_B196_uid218000 | cd09643 | cas9 |
| 527330434 | Streptococcus_lutetiensis_033_uid213397 | mkCas0193 | cas9 |
| 374338350 | Streptococcus_macedonicus_ACA_DC_198_uid81631 | cd09643 | cas9 |
| 397650022 | Streptococcus_mutans_GS_5_uid169223 | mkCas0193 | cas9 |
| 387785882 | Streptococcus_mutans_LJ23_uid162197 | mkCas0193 | cas9 |
| 290580220 | Streptococcus_mutans_NN2025_uid46353 | mkCas0193 | cas9 |
| 24379809 | Streptococcus_mutans_UA159_uid57947 | mkCas0193 | cas9 |
| 336064611 | Streptococcus_pasteurianus_ATCC_43144_uid68019 | cd09643 | cas9 |
| 410680443 | Streptococcus_pyogenes_A20_uid178106 | COG3513 | cas9 |
| 470200927 | Streptococcus_pyogenes_M1_476_uid193766 | COG3513 | cas9 |
| 15675041 | Streptococcus_pyogenes_M1_GAS_uid57845 | COG3513 | cas9 |
| 94990395 | Streptococcus_pyogenes_MGAS10270_uid58571 | COG3513 | cas9 |
| 94994317 | Streptococcus_pyogenes_MGAS10750_uid58575 | COG3513 | cas9 |
| 383479946 | Streptococcus_pyogenes_MGAS15252_uid158037 | COG3513 | cas9 |
| 383493861 | Streptococcus_pyogenes_MGAS_1882_uid158061 | COG3513 | cas9 |
| 94992340 | Streptococcus_pyogenes_MGAS2096_uid58573 | COG3513 | cas9 |
| 21910213 | Streptococcus_pyogenes_MGAS315_uid57911 | COG3513 | cas9 |
| 71910582 | Streptococcus_pyogenes_MGAS5005_uid58337 | COG3513 | cas9 |
| 71903413 | Streptococcus_pyogenes_MGAS6180_uid58335 | COG3513 | cas9 |
| 94988516 | Streptococcus_pyogenes_MGAS9429_uid58569 | COG3513 | cas9 |
| 209559356 | Streptococcus_pyogenes_NZ131_uid59035 | COG3513 | cas9 |
| 28896088 | Streptococcus_pyogenes_SSI_1_uid57895 | COG3513 | cas9 |
| 387783792 | Streptococcus_salivarius_JIM8777_uid162145 | cd09643 | cas9 |
| 386584496 | Streptococcus_suis_D9_uid162125 | cd09643 | cas9 |
| 389856936 | Streptococcus_suis_ST1_uid167482 | mkCas0193 | cas9 |
| 330833104 | Streptococcus_suis_ST3_uid66327 | cd09643 | cas9 |
| 55822627 | Streptococcus_thermophilus_CNRZ1066_uid58221 | cd09643 | cas9 |
| 386344353 | Streptococcus_thermophilus_JIM_8232_uid162157 | cd09643 | cas9 |
| 116627542 | Streptococcus_thermophilus_LMD_9_uid58327 | cd09643 | cas9 |
| 116628213 | Streptococcus_thermophilus_LMD_9_uid58327 | mkCas0193 | cas9 |
| 55820735 | Streptococcus_thermophilus_LMG_18311_uid58219 | cd09643 | cas9 |
| 387909441 | Streptococcus_thermophilus_MN_ZLW_002_uid166827 | cd09643 | cas9 |
| 387910220 | Streptococcus_thermophilus_MN_ZLW_002_uid166827 | mkCas0193 | cas9 |
| 386086348 | Streptococcus_thermophilus_ND03_uid162015 | cd09643 | cas9 |
| 386087120 | Streptococcus_thermophilus_ND03_uid162015 | mkCas0193 | cas9 |
| 389874754 | Tistrella_mobilis_KA081020_065_uid167486 | COG3513 | cas9 |
| 42525843 | Treponema_denticola_ATCC_35405_uid57583 | mkCas0193 | cas9 |
| 530892607 | Treponema_pedis_T_A4_uid215715 | COG3513, COG3513 | cas9 |
| 121608211 | Verminephrobacter_eiseniae_EF01_2_uid58675 | cd09643 | cas9 |
| 525888882 | Vibrio_parahaemolyticus_O1_K33_CDC_K4557_uid212977 | COG3513, COG3513 | cas9 |
| 525913263 | Vibrio_parahaemolyticus_O1_K33_CDC_K4557_uid212977 | COG3513 | cas9 |
| 525919586 | Vibrio_parahaemolyticus_O1_K33_CDC_K4557_uid212977 | COG3513, COG3513 | cas9 |
| 525927253 | Vibrio_parahaemolyticus_O1_K33_CDC_K4557_uid212977 | COG3513, COG3513 | cas9 |
| 325955459 | Weeksella_virosa_DSM_16922_uid63627 | cd09643, cd09643 | cas9 |
| 34557790 | Wolinella_succinogenes_DSM_1740_uid61591 | cd09643 | cas9 |
| 34557932 | Wolinella_succinogenes_DSM_1740_uid61591 | cd09704 | cas9 |
| 295136244 | Zunongwangia_profunda_SM_A87_uid48073 | COG3513, cd09643 | cas9 |
| 304313029 | gamma_proteobacterium_HdN1_uid51635 | cd09643 | cas9 |
| 189485058 | uncultured_Termite_group_1_bacterium_phylotype_Rs_D17_uid59059 | cd09643 | cas9 |
| 189485059 | uncultured_Termite_group_1_bacterium_phylotype_Rs_D17_uid59059 | cd09643 | cas9 |
| 189485225 | uncultured_Termite_group_1_bacterium_phylotype_Rs_D17_uid59059 | COG3513 | cas9 |
| 347536497 | Flavobacterium_branchiophilum_FL_15_uid73421 | COG3513, cd09643, COG3513 | cas9, cas9 |
| 365959402 | Flavobacterium_columnare_ATCC_49512_uid80731 | COG3513, cd09643, COG3513 | cas9, cas9 |
| 387132277 | Prevotella_intermedia_17_uid163151 | cd09643, COG3513, COG0188 | cas9, Type IIA |
| | | | topoisomerase |

Guide RNA (gRNA)

gRNA or sgRNA (single guide RNA) is engineered as a fusion between a crRNA and part of the transactivating CRISPR RNA (tracrRNA) sequence, which guides the Cas9 to a specific target DNA sequence that is complementary to the protospacer region. Guide RNA may include an expression cassette containing a chimeric RNA design with a long tracrRNA hybrid, short tracrRNA hybrid or a native CRISPR array+tracrRNA conformation. Chimeric gRNA combines the targeting specificity of the crRNA with the scaffolding properties of the tracrRNA into a single transcript. gRNA transient expression is controlled by species-specific higher plant RNA Polymerase III promoters such as those from the U6 or U3 snRNA gene family (Wang et al., 2008). gRNA transcript termination is controlled by a 6-20 nucleotide tract of poly dT as per Wang et al. (2008). gRNA expression cassettes are located on the same or different transient expression vectors from other components of the CRISPR-Cas system. gRNA transcripts may be synthesized in vitro and delivered directly into plant cells, independent of or in combination with gRNA transient expression vectors.

In some embodiments, the native S. pyogenes type II CRISPR-Cas system consists of a Crispr Associated (Cas9) nuclease and two disparate non-coding RNAs, trans-activating RNA (tracrRNA) and CRISPR RNA (crRNA). The RNA components of this system direct Cas9 nuclease to a sequence specific target in a genome. All three components can be expressed separately as tracrRNA and crRNA and Cas9 protein. The crRNA provides the target specificity and consists of a spacer sequence of 20 bases that are complementary to the target DNA (protospacer sequence) that is cleaved by Cas9 (Cong et al., 2013). It is noted that up to six base mismatches within the PAM-distal region of the spacer has been shown to be tolerated by Cas9 (Jinek et., 2012). Further, spacer sequence with <20 nucleotides of target sequence complementarity at the PAM-distal region can reduce off-target mutagenesis without impacting on-target cleavage efficiencies (Fu et al., Nat Biotechnol. 2014; 32 (3): 279-284. doi: 10.1038/nbt.2808. A programmable dual-RNA-guided DNA endonuclease in adaptive bacterial immunity. science, 337 (6096), 816-821.). (The tracrRNA acts as an RNA scaffold when associated with crRNA by way of RNA: RNA base pairing and it is this complex that associates with Cas9. The tracrRNA can be engineered to be shorter than 89 bases, as is the case in the Alt-R™ system developed by Integrated DNA Technologies (IDT). In this system tracrRNA as short as 67 bases have increased on-target performance when compared to native tracrRNA. When the crRNA and tracrRNA are artificially combined into a single fused functional RNA or single guide RNA (sgRNA) targeting of Cas9 protein can be greatly simplified over the native system. Similar to the native tracerRNA: crRNA complex, the engineered sgRNA guides the Cas9 to a specific target DNA sequence.

### Target Region

Guide RNAs contain two components that define specificity to a DNA target region, a proto-spacer and a proto-spacer adjacent motif (PAM). Proto-spacer sequence, typically 20 nucleotides but can vary based on the DNA target, provides DNA sequence specificity for the CRISPR-Cas complex. DNA targets also contain an NNG or NAG tri-nucleotide sequence (PAM) where N denotes any nucleotide, immediately 3′ or downstream of the proto-spacer.

### One Component Approach

Similar to Cong et al. (2013) and others, a simplified “one component approach” to CRISPR-Cas gene editing wherein a single transient expression construct contains all components of the CRISPR-Cas complex, i.e. both the gRNA and the Cas expressions cassettes. This allows for an easy modular design for targeting single or multiple loci in any given plant or crop. Targeting multiple loci can be achieved by simply swapping in the target-specific gRNA cassettes. Additionally, species specific promoters, terminators or other expressing enhancing elements can easily be shuttled in and out of “one component approach” transient vectors allowing for optimal expression of both gRNA and Cas protein in a species-specific manner.

### Two Component Approach

In the two-component approach, Cas and gRNA expression cassettes are located on different transient expression vectors. This allows for delivery of a CRISPR-Cas editing components separately, allowing for different ratios of gRNA to Cas within the same cell. Similar to the one component approach, the two-component approach also allows for promoters, terminators or other elements affecting expression of CRISPR-Cas components to be easily altered and allow targeting of DNA in a species-specific manner.

### Antibiotics

Another class of endonucleases are antibiotics which are DNA cleaving glycopeptides such as the bleomycin family of antibiotics are DNA cleaving glycopeptides which include bleomycin, zeocin, phleomycin, tallysomycin, pepleomycin and others which are further described herein.

### Other DNA-Modifying Molecules

Other DNA-modifying molecules may be used in targeted gene recombination. For example, peptide nucleic acids may be used to induce modifications to the genome of the target cell or cells (see, e.g., Ecker, U.S. Pat. No. 5,986,053 herein incorporated by reference). In brief, synthetic nucleotides comprising, at least, a partial peptide backbone, are used to target a homologous genomic nucleotide sequence. Upon binding to the double-helical DNA, or through a mutagen ligated to the peptide nucleic acid, modification of the target DNA sequence and/or recombination is induced to take place. Targeting specificity is determined by the degree of sequence homology between the targeting sequence and the genomic sequence.

In some embodiments of the methods and compositions of the present disclosure genes (such as the ACCase gene) may be targeted using triple helix forming oligonucleotides (TFO). TFOs may be generated synthetically, for example, by PCR or by use of a gene synthesizer apparatus. Additionally, TFOs may be isolated from genomic DNA if suitable natural sequences are found. TFOs may be used in a number of ways, including, for example, by tethering to a mutagen such as, but not limited to, psoralen or chlorambucil (see, e.g., Havre et al., Proc Nat'l Acad Sci, U.S.A. 90:7879-7883, 1993; Havre et al., J Virol 67:7323-7331, 1993; Wang et al., Mol Cell Biol 15:1759-1768, 1995; Takasugi et al., Proc Nat'l Acad Sci, U.S.A. 88:5602-5606, 1991; Belousov et al., Nucleic Acids Res 25:3440-3444, 1997). Furthermore, for example, TFOs may be tethered to donor duplex DNA (see, e.g., Chan et al., J Biol Chem 272:11541-11548, 1999). TFOs can also act by binding with sufficient affinity to provoke error-prone repair (Wang et al., Science 271:802-805, 1996).

The methods disclosed herein are not necessarily limited to the nature or type of DNA-modifying reagent which is used. For example, such DNA-modifying reagents release radicals which result in DNA strand breakage. Alternatively, the reagents alkylate DNA to form adducts which would block replication and transcription. In another alternative, the reagents generate crosslinks or molecules that inhibit cellular enzymes leading to strand breaks. Examples of DNA-modifying reagents which have been linked to oligonucleotides to form TFOs include, but are not limited to, indolocarbazoles, napthalene diimide (NDI), transplatin, bleomycin, analogues of cyclopropapyrroloindole, and phenanthodihydrodioxins. In particular, indolocarbazoles are topoisomerase I inhibitors. Inhibition of these enzymes results in strand breaks and DNA protein adduct formation (Arimondo et al., Bioorganic and Medicinal Chem. 8, 777, 2000). NDI is a photooxidant that can oxidize guanines which could cause mutations at sites of guanine residues (Nunez, et al., Biochemistry, 39, 6190, 2000). Transplatin has been shown to react with DNA in a triplex target when the TFO is linked to the reagent. This reaction causes the formation of DNA adducts which would be mutagenic (Columbier, et al., Nucleic Acids Research, 24:4519, 1996). Bleomycin is a DNA breaker, widely used as a radiation mimetic. It has been linked to oligonucleotides and shown to be active as a breaker in that format (Sergeyev, Nucleic Acids Research 23, 4400, 1995; Kane, et al., Biochemistry, 34, 16715, 1995). Analogues of cyclopropapyrroloindole have been linked to TFOs and shown to alkylate DNA in a triplex target sequence. The alkylated DNA would then contain chemical adducts which would be mutagenic (Lukhtanov, et al., Nucleic Acids Research, 25, 5077, 1997). Phenanthodihydrodioxins are masked quinones that release radical species upon photoactivation. They have been linked to TFOs and have been shown to introduce breaks into duplex DNA on photoactivation (Bendinskas et al., Bioconjugate Chem. 9, 555, 1998).

Other methods of inducing modifications and/or recombination are contemplated by the present disclosure. For example, another embodiment involves the induction of homologous recombination between an exogenous DNA fragment and the targeted gene (see e.g., Capecchi et al., Science 244:1288-1292, 1989) or by using peptide nucleic acids (PNA) with affinity for the targeted site. Still other methods include sequence specific DNA recognition and targeting by polyamides (see e.g., Dervan et al., Curr Opin Chem Biol 3:688-693, 1999; Biochemistry 38:2143-2151, 1999) and the use nucleases with site specific activity (e.g., zinc finger proteins, TALENs, Meganucleases and/or CRISPRs).

The present disclosure is not limited to any particular frequency of modification and/or recombination. In some embodiments the methods disclosed herein result in a frequency of modification in the target nucleotide sequence of from 0.01% to 3%. Nonetheless, any frequency (i.e., between 0% and 100%) of modification and/or recombination is contemplated to be within the scope of the present disclosure. The frequency of modification and/or recombination is dependent on the method used to induce the modification and/or recombination, the cell type used, the specific gene targeted, and the DNA mutating reagent used, if any. Additionally, the method used to detect the modification and/or recombination, due to limitations in the detection method, may not detect all occurrences of modification and/or recombination. Furthermore, some modification and/or recombination events may be silent, giving no detectable indication that the modification and/or recombination has taken place. The inability to detect silent modification and/or recombination events gives an artificially low estimate of modification and/or recombination. Because of these reasons, and others, the disclosure is not necessarily limited to any particular modification and/or recombination frequency. In one embodiment, the frequency of modification and/or recombination is between 0.01% and 100%. In another embodiment, the frequency of modification and/or recombination is between 0.01% and 50%. In yet another embodiment, the frequency of modification and/or recombination is between 0.1% and 10%. In still yet another embodiment, the frequency of modification and/or recombination is between 0.1% and 5%.

The term “frequency of mutation” as used herein in reference to a population of cells which are treated with a DNA-modifying molecule that is capable of introducing a mutation into a target site in the cells' genome, refers to the number of cells in the treated population which contain the mutation at the target site as compared to the total number of cells which are treated with the DNA-modifying molecule. For example, with respect to a population of cells which is treated with the DNA-modifying molecule TFO tethered to psoralen which is designed to introduce a mutation at a target site in the cells' genome, a frequency of mutation of 5% means that of a total of 100 cells which are treated with TFO-psoralen, 5 cells contain a mutation at the target site.

Although the present disclosure is not necessarily limited to any degree of precision in the modification and/or recombination of DNA in the cell, it is contemplated that some embodiments of the present disclosure require higher degrees of precision, depending on the desired result. For example, the specific sequence changes required for gene repair (e.g., particular base changes) require a higher degree of precision as compared to producing a gene knockout wherein only the disruption of the gene is necessary. With the methods of the present disclosure, achievement of higher levels of precision in modification and/or homologous recombination techniques is greater than with prior art methods.

Delivery of Gene Repair Oligonucleobases into Plant Cells

Any commonly known method used to transform a plant cell can be used for delivering the gene repair oligonucleobases. Illustrative methods are listed below. The methods and compositions herein may involve any of many methods to transfect the cells with the DNA-modifying reagent or reagents. Methods for the introduction of DNA modifying reagents into a cell or cells are well known in the art and include, but are not limited to, microinjection, electroporation, passive adsorption, calcium phosphate-DNA co-precipitation, DEAE-dextran-mediated transfection, polybrene-mediated transfection, liposome fusion, lipofectin, nucleofection, protoplast fusion, retroviral infection, biolistics (i.e., particle bombardment) and the like.

The use of metallic microcarriers (microspheres) for introducing large fragments of DNA into plant cells having cellulose cell walls by projectile penetration is well known to those skilled in the relevant art (henceforth biolistic delivery). U.S. Pat. Nos. 4,945,050; 5,100,792 and 5,204,253 describe general techniques for selecting microcarriers and devices for projecting them.

Specific conditions for using microcarriers in the methods disclosed herein may include the conditions described in International Publication WO 99/07865. In an illustrative technique, ice cold microcarriers (60 mg/ml), mixed duplex oligonucleotide (60 mg/ml) 2.5 M CaCl₂ and 0.1 M spermidine are added in that order; the mixture gently agitated, e.g., by vortexing, for 10 minutes and then left at room temperature for 10 minutes, whereupon the microcarriers are diluted in 5 volumes of ethanol, centrifuged and resuspended in 100% ethanol. Good results can be obtained with a concentration in the adhering solution of 8-10 μg/μl microcarriers, 14-17 μg/ml mixed duplex oligonucleotide, 1.1-1.4 M CaCl₂) and 18-22 mM spermidine. Optimal results were observed under the conditions of 8 μg/μl microcarriers, 16.5 μg/ml mixed duplex oligonucleotide, 1.3 M CaCl₂) and 21 mM spermidine.

Gene repair oligonucleobases can also be introduced into plant cells using microfibers to penetrate the cell wall and cell membrane. U.S. Pat. No. 5,302,523 to Coffee et al. describes the use of silicon carbide fibers to facilitate transformation of suspension maize cultures of Black Mexican Sweet. Any mechanical technique that can be used to introduce DNA for transformation of a plant cell using microfibers can be used to deliver gene repair oligonucleobases for transmutation.

An illustrative technique for microfiber delivery of a gene repair oligonucleobase is as follows: Sterile microfibers (2 μg) are suspended in 150 μl of plant culture medium containing about 10 μg of a mixed duplex oligonucleotide. A suspension culture is allowed to settle and equal volumes of packed cells and the sterile fiber/nucleotide suspension are vortexed for 10 minutes and plated. Selective media are applied immediately or with a delay of up to about 120 hours as is appropriate for the particular trait.

In an alternative embodiment, the gene repair oligonucleobases can be delivered to the plant cell by electroporation of a protoplast derived from a plant part. The protoplasts are formed by enzymatic treatment of a plant part, particularly a leaf, according to techniques well known to those skilled in the art. See, e.g., Gallois et al., 1996, in Methods in Molecular Biology 55:89-107, Humana Press, Totowa, N.J.; Kipp et al., 1999, in Methods in Molecular Biology 133:213-221, Humana Press, Totowa, NJ. The protoplasts need not be cultured in growth media prior to electroporation. Illustrative conditions for electroporation are 300,000 protoplasts in a total volume of 0.3 ml with a concentration of gene repair oligonucleobase of between 0.6-4 μg/ml.

In an alternative embodiment, nucleic acids are taken up by plant protoplasts in the presence of the membrane-modifying agent polyethylene glycol, according to techniques well known to those skilled in the art. In another alternative embodiment, the gene repair oligonucleobases can be delivered by injecting it with a microcapillary into plant cells or into protoplasts.

In an alternative embodiment, nucleic acids are embedded in microbeads composed of calcium alginate and taken up by plant protoplasts in the presence of the membrane-modifying agent polyethylene glycol (see, e.g., Sone et al., 2002, Liu et al., 2004).

In an alternative embodiment, nucleic acids frozen in water and introduced into plant cells by bombardment in the form of microparticles (see, e.g., Gilmore, 1991, U.S. Pat. No. 5,219,746; Brinegar et al.).

In an alternative embodiment, nucleic acids attached to nanoparticles are introduced into intact plant cells by incubation of the cells in a suspension containing the nanoparticle (see, e.g., Pasupathy et al., 2008) or by delivering them into intact cells through particle bombardment or into protoplasts by co-incubation (see, e.g., Torney et al., 2007).

In an alternative embodiment, nucleic acids complexed with penetrating peptides and delivered into cells by co-incubation (see, e.g., Chugh et al., 2008, WO 2008148223 A1; Eudes and Chugh).

In an alternative embodiment, nucleic acids are introduced into intact cells through electroporation (see, e.g., He et al., 1998, U.S. 2003/0115641 A1, Dobres et al.).

In an alternative embodiment, nucleic acids are delivered into cells of dry embryos by soaking them in a solution with nucleic acids (see, e.g., Töpfer et al., 1989; Senaratna et al., 1991) or in other embodiments are introduced by Cellsqueeze (SQZ Biotech).

### Methods of Reducing Polypeptide Activity and Other Mutagenesis Techniques

Certain aspects of the present disclosure relate to reducing levels and/or activity of a polypeptide (, an ACCase polypeptide). Methods of modifying decreasing the quantity/level or the activity of one or more polypeptides of the present disclosure are well-known in the art and are described herein.

Cells (, plant cells) of the present disclosure may contain one or more polypeptides with decreased activity as compared to a corresponding control cell, such as a wild-type cell. In some embodiments, one or more ACCase proteins have decreased activity in a host cell as compared to a corresponding control cell. Methods of decreasing the expression, abundance, and/or activity of a polypeptide are well-known in the art and are described herein.

In some embodiments, decreasing the activity of a polypeptide such as, for example, one or more ACCase proteins involves decreasing the expression of a nucleic acid encoding the polypeptide.

Decreasing the expression of a nucleic acid may be accomplished by introducing a genetic mutation into a target nucleic acid. Mutagenesis approaches may be used to disrupt or “knockout” the expression of a target gene by generating mutations. In some embodiments, the mutagenesis results in a partial deletion of the target gene. In other embodiments, the mutagenesis results in a complete deletion of the target gene. Methods of mutagenizing microorganisms are well known in the art and include, for example, random mutagenesis and site-directed mutagenesis to induce mutations. Examples of methods of random mutagenesis include, for example, chemical mutagenesis (e.g., using ethane methyl sulfonate), insertional mutagenesis, and irradiation.

One method for reducing or inhibiting the expression of a target gene is by genetically modifying the target gene and introducing it into the genome of a host cell to replace the wild-type version of the gene by homologous recombination (for example, as described in U.S. Pat. No. 6,924,146).

Another method for reducing or inhibiting the expression of a target gene is by insertion mutagenesis using the T-DNA of Agrobacterium tumefaciens, or transposons (see Winkler et al., Methods Mol. Biol. 82:129-136, 1989, and Martienssen Proc. Natl. Acad. Sci. 95:2021-2026, 1998). After generating the insertion mutants, the mutants can be screened to identify those containing the insertion in a target gene. Methods to disrupt a target gene by insertional mutagenesis are described in for example, U.S. Pat. No. 5,792,633. Methods to disrupt a target gene by transposon mutagenesis are described in for example, U.S. Pat. No. 6,207,384.

A further method to disrupt a target gene is by use of the cre-lox system (for example, as described in U.S. Pat. No. 4,959,317).

Another method to disrupt a target gene is by use of PCR mutagenesis (for example, as described in U.S. Pat. No. 7,501,275).

Endogenous gene expression may also be reduced or inhibited by means of RNA interference (RNAi), which uses a double-stranded RNA having a sequence identical or similar to the sequence of the target gene. RNAi may include the use of micro-RNA, such as artificial miRNA, to suppress expression of a gene.

RNAi is the phenomenon in which when a double-stranded RNA having a sequence identical or similar to that of the target gene is introduced into a cell, the expressions of both the inserted exogenous gene and target endogenous gene are suppressed. The double-stranded RNA may be formed from two separate complementary RNAs or may be a single RNA with internally complementary sequences that form a double-stranded RNA.

Thus, in some embodiments, reduction or inhibition of gene expression is achieved using RNAi techniques. For example, to achieve reduction or inhibition of the expression of a DNA encoding a protein using RNAi, a double-stranded RNA having the sequence of a DNA encoding the protein, or a substantially similar sequence thereof (including those engineered not to translate the protein) or fragment thereof, is introduced into a host cell of interest. As used herein, RNAi and dsRNA both refer to gene-specific silencing that is induced by the introduction of a double-stranded RNA molecule, see e.g., U.S. Pat. Nos. 6,506,559 and 6,573,099, and includes reference to a molecule that has a region that is double-stranded, e.g., a short hairpin RNA molecule. The resulting cells may then be screened for a phenotype associated with the reduced expression of the target gene, e.g., reduced cellulase expression, and/or by monitoring steady-state RNA levels for transcripts of the target gene. Although the sequences used for RNAi need not be completely identical to the target gene, they may be at least 70%, 80%, 90%, 91%, 92%, 93%, 94%, 95%, 96%, 97%, 98%, 99%, or more identical to the target gene sequence. See, e.g., U.S. Patent Application Publication No. 2004/0029283. The constructs encoding an RNA molecule with a stem-loop structure that is unrelated to the target gene and that is positioned distally to a sequence specific for the gene of interest may also be used to inhibit target gene expression. See, e.g., U.S. Patent Application Publication No. 2003/0221211.

The RNAi nucleic acids may encompass the full-length target RNA or may correspond to a fragment of the target RNA. In some cases, the fragment will have fewer than 100, 200, 300, 400, or 500 nucleotides corresponding to the target sequence. In addition, in some aspects, these fragments are at least, e.g., 50, 100, 150, 200, or more nucleotides in length. Interfering RNAs may be designed based on short duplexes (i.e., short regions of double-stranded sequences). Typically, the short duplex is at least about 15, 20, or 25-50 nucleotides in length (e.g., each complementary sequence of the double stranded RNA is 15-50 nucleotides in length), often about 20-30 nucleotides, e.g., 20, 21, 22, 23, 24, 25, 26, 27, 28, 29, or 30 nucleotides in length. In some cases, fragments for use in RNAi will correspond to regions of a target protein that do not occur in other proteins in the organism or that have little similarity to other transcripts in the organism, e.g., selected by comparison to sequences in analyzing publicly-available sequence databases. Similarly, RNAi fragments may be selected for similarity or identity with a conserved sequence of a gene family of interest, such as those described herein, so that the RNAi targets multiple different gene transcripts containing the conserved sequence.

RNAi may be introduced into a host cell as part of a larger DNA construct. Often, such constructs allow stable expression of the RNAi in cells after introduction, e.g., by integration of the construct into the host genome. Thus, expression vectors that continually express RNAi in cells transfected with the vectors may be employed for this disclosure. For example, vectors that express small hairpin or stem-loop structure RNAs, or precursors to microRNA, which get processed in vivo into small RNAi molecules capable of carrying out gene-specific silencing (Brummelkamp et al., Science 296:550-553, (2002); and Paddison, et al., Genes & Dev. 16:948-958, (2002)) can be used. Post-transcriptional gene silencing by double-stranded RNA is discussed in further detail by Hammond et al., Nature Rev Gen 2:110-119, (2001); Fire et al., Nature 391:806-811, (1998); and Timmons and Fire, Nature 395:854, (1998).

Methods for selection and design of sequences that generate RNAi are well-known in the art (e.g., U.S. Pat. Nos. 6,506,559; 6,511,824; and 6,489,127).

A reduction or inhibition of gene expression in a host cell of a target gene may also be obtained by introducing into host cells antisense constructs based on a target gene nucleic acid sequence. For antisense suppression, a target sequence is arranged in reverse orientation relative to the promoter sequence in the expression vector. The introduced sequence need not be a full-length cDNA or gene and need not be identical to the target cDNA or a gene found in the cell to be transformed. Generally, however, where the introduced sequence is of shorter length, a higher degree of homology to the native target sequence is used to achieve effective antisense suppression. In some aspects, the introduced antisense sequence in the vector will be at least 30 nucleotides in length, and improved antisense suppression will typically be observed as the length of the antisense sequence increases. In some aspects, the length of the antisense sequence in the vector will be greater than 100 nucleotides. Transcription of an antisense construct as described results in the production of RNA molecules that are the reverse complement of mRNA molecules transcribed from an endogenous target gene. Suppression of a target gene expression can also be achieved using a ribozyme. The production and use of ribozymes are disclosed in U.S. Pat. Nos. 4,987,071 and 5,543,508.

Expression cassettes containing nucleic acids that encode target gene expression inhibitors, e.g., an antisense or siRNA, can be constructed using methods well known in the art. Constructs include regulatory elements, including promoters and other sequences for expression and selection of cells that express the construct. Typically, fungal and/or bacterial transformation vectors include one or more cloned coding sequences (genomic or cDNA) under the transcriptional control of 5′ and 3′ regulatory sequences and a dominant selectable marker. Such transformation vectors typically also contain a promoter (e.g., a regulatory region controlling inducible or constitutive, environmentally- or developmentally-regulated expression), a transcription initiation start site, an RNA processing signal (such as intron splice sites), a transcription termination site, and/or a polyadenylation signal.

In certain embodiments, a portion of the target nucleic acid may be modified, such as the region encoding the catalytic domain, the coding region, or a control sequence required for expression of the coding region. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification may include, for example, a leader sequence, a propeptide sequence, a signal sequence, a transcription terminator, and a transcriptional activator.

### Plants of the Present Disclosure

The methods and compositions described herein may in certain aspects and embodiments be applicable to plants generally. For example, in some aspects and/or embodiments a plant species may be selected from the group consisting of corn, (maize), wheat (including but not limited to Triticum spp., Triticum aestivum, Triticum durum Triticum timopheevii, Triticum monococcum, Triticum spelta, Triticum zhukovskyi and Triticum urartu and hybrids thereof), barley (including but not limited to Hordeum vulgare L., Hordeum comosum, Hordeum depressum, Hordeum intercedens, Hordeum jubatum, Hordeum marinum, Hordeum marinum, Hordeum parodii, Hordeum pusillum, Hordeum secalinum, and Hordeum spontaneum), rice (including but not limited to Oryza sativa subsp. indica, Oryza sativa subsp. japonica, Oryza sativa subsp. javanica, Oryza sativa subsp. glutinosa (glutinous rice), Oryza sativa aromatica group (e.g., basmati), and Oryza sativa (floating rice group)), sorghum (e.g., Sorghum bicolor), foxtail millet (e.g., Setaria italica), switchgrass (e.g., Panicum virgatum), Kentucky blue-grass (e.g., Poa pratensis), banana, onion, sugar cane, triticale, rye (including but not limited to Secale sylvestre, Secale strictum, Secale cereale, Secale vavilovii, Secale africanum, Secale ciliatoglume, Secale ancestrale, and Secale montanum), perennial ryegrass (e.g., Lolium perenne), oats (Avena sativa), turf (including but not limited to Turf grass include Zoysia japonica, Agrostris palustris, Poa pratensis, Poa annua, Digitaria sanguinalis, Cyperus rotundus, Kyllinga brevifolia, Cyperus amuricus, Erigeron canadensis, Hydrocotyle sibthorpioides, Kummerowia striata, Euphorbia humifusa, and Viola arvensis) and forage grasses, lotus, tulip, iris, lily, nut-producing plants insofar as they are not already specifically mentioned. The plant species may be selected from the group consisting of monocotyledonous plants of the grass family Poaceae. The family Poaceae may be divided into two major clades, the clade containing the subfamilies Bambusoideae, Ehrhartoideae, and Pooideae (the BEP clade) and the clade containing the subfamilies Panicoideae, Arundinoideae, Chloridoideae, Centothecoideae, Micrairoideae, Aristidoideae, and Danthonioideae (the PACCMAD clade). The subfamily Bambusoideae includes tribe Oryzeae. The plant species may relate to plants of the BEP clade, in particular plants of the subfamilies Bambusoideae and Ehrhartoideae. The BET clade includes subfamilies Bambusoideae, Ehrhartoideae, and group Triticodae and no other subfamily Pooideae groups. BET crop plants are plants grown for food or forage that are members of BET subclade, for example barley, corn, etc. In some embodiments the plant belongs to the genus Oryza. The two most frequently cultivated species are Oryza sativa and Oryza glaberrima, with O. sativa being the most frequently cultivated domestic rice. In addition to these two domestic species, the genus Oryza contains more than 20 wild species-any of which could be a plant of the present disclosures. In certain embodiments the plant is Oryza rufipogon (“red rice” also referred to as Oryza sativa subsp. rufipogon).

In various embodiments, plants as disclosed herein are principally focused on monocotyledonous plants including any woody plant species that grows as a tree or shrub, any herbaceous species, or any species that produces edible fruits, seeds or vegetables, or any species that produces colorful or aromatic flowers. For example, the plant maybe selected from a species of plant from the group consisting of canola, sunflower, corn, tobacco, sugar beet, cotton, maize, wheat, barley, rice, alfalfa, sorghum, foxtail millet, switchgrass, Kentucky blue-grass, tomato, mango, peach, apple, pear, strawberry, banana, melon, cassava, potato, carrot, lettuce, onion, soy bean, soya spp, sugar cane, pea, chickpea, field pea, fava bean, lentils, turnip, rutabaga, brussels sprouts, lupin, cauliflower, kale, field beans, poplar, pine, eucalyptus, grape, citrus, triticale, alfalfa, rye, perennial ryegrass, oats, turf and forage grasses, flax, oilseed rape, mustard, cucumber, morning glory, balsam, pepper, eggplant, marigold, lotus, cabbage, daisy, carnation, tulip, iris, lily, and nut producing plants insofar as they are not already specifically mentioned.

According to the present description, substantially normal growth of a plant, plant organ, plant tissue or plant cell is defined as a growth rate or rate of cell division of the plant, plant organ, plant tissue, or plant cell that is at least 35%, at least 50%, at least 60%, or at least 75% of the growth rate or rate of cell division in a corresponding plant, plant organ, plant tissue or plant cell expressing the corresponding ACCase reference protein sequence that lacks the sequence modifications being tested.

Growth may be assessed by determining the dry biomass of plants, organs, seeds, or tissues. By way of example, a quadrat of vegetation is selected, and appropriate plants, cells, or seeds within the quadrat are harvested. The size of the quadrat is selected to capture any variation in biomass in the area of interest. The sample is weight when fresh, dried at 60-70° C., and the dried material is re-weighed. Biomass may be expressed as fresh weight, dry weight, or % dry matter (Dry Wt./Fresh Wt.*100, where the “Dry Wt.” is the weight of the sample after oven drying and “Fresh Wt.” is the weight of the sample recorded in the field). Dry weight is preferred, as this eliminates fluctuations in weight caused by water content.

In certain embodiments, substantially normal growth is tested against the corresponding sequences for that species recited in the present specification. By way of example, the following ACCase sequences are recited herein which may be modified according to the invention and serve as the corresponding reference sequence:
Blackgrass ACCase SEQ ID NO: 2
Rice (Cypress) ACCase SEQ ID NO: 4
Rice (Japonica) ACCase SEQ ID NO: 6
Wheat ACCase SEQ ID NO: 8
Corn ACCase SEQ ID NO: 10, 12, or 14
Foxtail millet ACCase 1b SEQ ID NO: 16, 18, or 20
Sorghum ACCase 1b SEQ ID NO: 22
Brachypodium distachyon ACCase SEQ ID NO: 23.
This list is not limiting, and other reference protein sequences are recited in the present specification.

According to the present description, substantially normal development of a plant, plant organ, plant tissue or plant cell is defined as the occurrence of one or more developmental events in the plant, plant organ, plant tissue or plant cell that are substantially the same as those occurring in a corresponding plant, plant organ, plant tissue or plant cell expressing the wild-type ACCase protein.

According to the present description plant organs include, but are not limited to, leaves, stems, roots, vegetative buds, floral buds, meristems, embryos, cotyledons, endosperm, sepals, petals, pistils, carpels, stamens, anthers, microspores, pollen, pollen tubes, ovules, ovaries and fruits, or sections, slices or discs taken therefrom. Plant tissues include, but are not limited to, callus tissues, ground tissues, vascular tissues, storage tissues, meristematic tissues, leaf tissues, shoot tissues, root tissues, gall tissues, plant tumor tissues, and reproductive tissues. Plant cells include, but are not limited to, isolated cells with cell walls, variously sized aggregates thereof, and protoplasts.

Plants of the present disclosure include those plants that have increased resistance or tolerance to a member of the aryloxyphenoxypropanoate, cyclohexanedione, and/or phenylpyrazoline families such as one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof; and exhibits substantially normal growth or development of the plant, its organs, tissues or cells, as compared to the corresponding wild-type plant or cell. Preferably, the mutated plant is resistant to one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof; and has substantially the same catalytic activity as compared to the wild-type ACCase protein. For example, the present disclosure includes Oryza plants that have increased resistance or tolerance to clethodim.

Plants which are substantially “resistant” to the one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof exhibit few, if any, necrotic, lytic, chlorotic or other lesions, when subjected to one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof at concentrations and rates which are typically employed by the agrochemical community to kill weeds in the field. Plants which are resistant to an herbicide are also tolerant of the herbicide.

Plants are substantially “tolerant” to one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof when they are subjected to it and provide a dose/response curve which is shifted to the right when compared with that provided by similarly subjected non-tolerant like plant. Such dose/response curves have “dose” plotted on the X-axis and “percentage kill”, “herbicidal effect”, etc., plotted on the y-axis. Tolerant plants will require more herbicide than non-tolerant like plants in order to produce a given herbicidal effect. The terms “resistant” and “tolerant” are to be construed as “tolerant and/or resistant” within the context of the present application.

In some embodiments, plants of the present disclosure with one or more mutations in an ACCase gene have increased resistance or tolerance to an herbicide as compared to a corresponding control plant (e.g., a plant of the same species that does not have any mutations in an ACCase gene, such as a wild-type plant).

### Generation of Plants

Tissue culture of various tissues of plant species and regeneration of plants therefrom is known. For example, the propagation of a canola cultivar by tissue culture is described in any of the following but not limited to any of the following: Li et al., “Somatic embryogenesis in quite a direct way in cultures of mesophyll protoplasts of Brassica napus L.”, Plant Cell Reports 1:209-211, 1982; Chuong et al., “A Simple Culture Method for Brassica hypocotyls Protoplasts,” Plant Cell Reports 4:4-6, 1985; Barsby et al., “A Rapid and Efficient Alternative Procedure for the Regeneration of Plants from Hypocotyl Protoplasts of Brassica napus,” Plant Cell Reports (Spring, 1996); Kartha et al., “In vitro Plant Formation from Stem Explants of Rape,” Physiol. Plant, 31:217-220, 1974; Narasimhulu et al., “Species Specific Shoot Regeneration Response of Cotyledonary Explants of Brassicas,” Plant Cell Reports (Spring 1988); Sun et al., “Cotyledon-derived diploid and haploid protoplast culture and diploid plant regeneration in Brassica napus cv. ‘Topas’,” Can. J. Bot. 76:530-541, 1998; Swanson, “Microspore Culture in Brassica,” Methods in Molecular Biology, Vol. 6, Chapter 17, p. 159, 1990.

Further reproduction of the variety can occur by tissue culture and regeneration. Tissue culture of various tissues of soybeans and regeneration of plants therefrom is well known and widely published. For example, see Komatsuda et al., “Genotype X Sucrose Interactions for Somatic Embryogenesis in Soybeans,” Crop Sci. 31:333-337, 1991; Stephens et al., “Agronomic Evaluation of Tissue-Culture-Derived Soybean Plants,” Theor. Appl. Genet. 82:633-635, 1991; Komatsuda et al., “Maturation and Germination of Somatic Embryos as Affected by Sucrose and Plant Growth Regulators in Soybeans Glycine gracilis Skvortz and Glycine max (L.) Merr.” Plant Cell, Tissue and Organ Culture, 28:103-113, 1992; Dhir et al., “Regeneration of Fertile Plants from Protoplasts of Soybean (Glycine max L. Merr.); Genotypic Differences in Culture Response,” Plant Cell Reports 11:285-289, 1992; Pandey et al., “Plant Regeneration from Leaf and Hypocotyl Explants of Glycine wightii (W. and A.) VERDC. var. longicauda,” Japan J. Breed. 42:1-5, 1992; and Shetty et al., “Stimulation of In Vitro Shoot Organogenesis in Glycine max (Merrill.) by Allantoin and Amides,” Plant Science 81:245-251, 1992. The disclosures of U.S. Pat. No. 5,024,944 issued Jun. 18, 1991, to Collins et al., and U.S. Pat. No. 5,008,200 issued Apr. 16, 1991, to Ranch et al., are hereby incorporated herein in their entirety by reference.

Certain aspects of the present disclosure also related to plants derived from plants having one or more mutations in a nucleic acid (e.g., an ACCase gene) of the present disclosure. For example, plants having one or more ACCase mutations may be crossed with the same or different plants to give rise to an F₁ progeny plant, where at least one of the parents of the F1 progeny plant had the one or more ACCase mutations. These F1 plants can be further self-crossed or crossed with a different plant line and resulting F2 progeny can be screened for one or more ACCase mutations.

### Herbicide Tolerance

Methods to measure herbicide tolerance of mutations listed here within are well known in the art. Joachimiak M. et al., Proc Natl Acad Sci U.S.A. 1997 Sep. 2; 94 (18): 9990-5 and WO/2013/074524 and WO/2013/074524 each describe methods to complement a yeast (Saccharomyces cerevisiae) strain lacking the native acc1 gene with either a wt or mutated form of a chimeric plastidal ACCase gene from a plant such as rice (Oryza sativa). Methods to introduce one or more mutations listed herein into the chimeric ACCase gene and subsequently complement the acc lacking yeast strain are well known by an individual skilled in the art. In the art assays are known for measuring tolerance of a mutated plant ACCase gene versus a herbicide that is a member of the aryloxyphenoxypropanoate, cyclohexanedione, and/or phenylpyrazoline families of herbicides such as clethodim, sethoxydim, fluazifop, haloxyfop, quizalofop, and pinoxaden (Joachimiak M. et al., Proc Natl Acad Sci U.S.A. 1997 Sep. 2; 94 (18): 9990-5 and WO/2013/074524).

Methods to measure tolerance of whole plants, such as rice (Oryza sativa), containing one or more mutations listed herein are known in the art. WO/2011/028833, WO/2013/016210, WO/2012/106321 and Neve and Powles, Heredity 2005 95, 485-492 detail procedures to assess tolerance of whole plants containing one or more ACCase mutations derived from random mutagenesis, soma clonal variation and transgenic procedures and such procedures are general applicable to plants produced by essentially any other method known in the art. The assays can be used by an individual skilled in the art to determine the tolerance of a plant with any one or more mutations listed herein versus a herbicide that is a member of the aryloxyphenoxypropanoate, cyclohexanedione, and/or phenylpyrazoline families of herbicides such as clethodim, sethoxydim, fluazifop, haloxyfop, quizalofop, and pinoxaden.

Plants and plant cells can be tested for resistance or tolerance to an herbicide using commonly known methods in the art, e.g., by growing the plant or plant cell in the presence of an herbicide and measuring the rate of growth as compared to the growth rate in the absence of the herbicide. Herbicide resistance is the inherited ability of an individual plant to survive exposure to a particular herbicide. Herbicide tolerance is the inherited ability of a species to survive and reproduce following herbicide treatment. See, e.g., hracglobal.com/herbicide-resistance/overview.

Herbicide resistance is measured by a “damage rating” and determining a level of the DR indicative of damage that impacts the agronomic characteristics of the tested plant. Assessment of herbicide dose response is based upon exposure of test and control plants at the normal field rate of application, and amounts above and below this rate, and assessing common symptoms of herbicide injury such as chlorosis, dieback, epicormic or adventitious sprouting, abnormal growth patterns, stunting, wilting, death, and/or the cupping, strapping, curling, or abnormal thickening of foliage, to determine an estimate of crop injury. The aim is to assess as accurately as possible the decrease in growth (density, biomass, height, etc.) as compared to a control, untreated plant. An arbitrary figure, such as DR₅₀, DR₇₀, DR₇₅, DR₈₀, DR₈₅, DR₉₀, DR₉₅, or DR₁₀₀, which refer to 50%, 70%, 75%, 80%, 85%, 90%, 95% or 100% severe injury or death, may be used as a threshold to assign a value of susceptibility. The application rate, or “dose,” at which the threshold is crossed would be increased in a resistant cultivar as compared to its control. Similarly with regard to herbicide tolerance, a grain (seed) yield of 50%, 70%, 75%, 80%, 85%, 90%, 95% or 100% of the parental wildtype may be taken as an arbitrary measure of fertility.

### EXEMPLARY EMBODIMENTS

In addition to the aspects and embodiments described and provided elsewhere in this disclosure, the following non-limiting list of particular embodiments are specifically contemplated.
1. A plant or plant cell having a mutation in an ACCase gene such that the mutated gene encodes an ACCase protein comprising one or more mutations resulting in one or more amino acid substitutions corresponding to the group consisting of the mutations listed on Table 1.
2. A plant or plant cell having a mutation in an ACCase gene such that the mutated gene encodes an ACCase protein comprising one or more mutations resulting in a substitution corresponding to a change selected from the group consisting of the mutations listed on Table 2.
3. A plant or plant cell having a mutation in an ACCase gene such that the mutated gene encodes an ACCase protein comprising one or more mutations resulting in a substitution corresponding to a change selected from the group consisting of the mutations listed on Table 3.
4. A plant or plant cell having a mutation in an ACCase gene such that the mutated gene encodes an ACCase protein comprising one or more amino acid substitutions at one or more amino acid positions corresponding to those selected from the group consisting of N1460, V1529, L1543, L1555, C1575, S1605, G1605-6A*, N1607, L1649, R1672, D1681, F1683, V1715, D1725, R1734, M1738, T1739, A1752, Q1756, 11766, K1772, N1780, 11781, G1783, S1784, A1785, A1786, S1792, I1811, L1818, Q1824, A1837, K1840, V1864, P1870, S1922, K1930, K1938, E1946, A1957, V1966, I1967, M1974, M1975, V1992, W1999, A2004, W2027, E2039, I2041, V2049, A2059, A2067, W2074, V2075, D2078, S2079, K2080, I2081, P2083, R2085, C2088, K2095, G2096, N2097, V2098, E2112, D2127, I2149, E2150, K2154, Q2155, L2159, E2169, A2178, A2179, V2182, F2196, H2223, A2226, K2232, A2241, S2244, F2253, V2254, R2257, N2262, K2264, I2267, S2276, S2280, S2297, L2299, K2302, and K2317 of SEQ ID NO: 2.
5. A plant or plant cell having a mutation in an ACCase gene such that the mutated gene encodes an ACCase protein comprising one or more amino acid substitutions corresponding to those selected from the group consisting of N1460S, V1529M, L1543F, L1543V, L1555P, C1575S, S1605Y, G1605-6A*, N1607S, L1649M, R1672I, D1681E, D1681G, F1683L, V1715A, D1725E, R1734G, M1738L, T1739S, A1752T, Q1756P, Q1756E, I1766V, K1772E, K1772N, N1780D, I1781L, I1781T, I1781V, I1781A, G1783C, S1784G, S1784N, A1785G, A1786P, S1792L, I1811N, L1818H, L1818I, Q1824P, A1837V, K1840M, K1840R, V1864F, P1870R, S1922G, K1930N, K1938R, E1946G, E1946D, E1946V, A1957T, V1966L, I1967V, M1974T, M1975T, V1992D, W1999C, W1999G, W1999L, W1999S, A2004V, W2027C, W2027R, E2039D, E2039G, I2041D, I2041N, I2041V, V2049F, V2049I, V2049L, V2049T, V2049C, V2049A, V2049S, A2059V, A2067S, W2074L, V2075M, V2075G, V2075I, V2075L, D2078G, D2078K, D2078T, D2078V, S2079F, S2079P, K2080E, P2083N, P2083Q, P2083S, P2083T, R2085S, C2088M, C2088N, C2088R, C2088W, C2088F, C2088G, C2088H, C2088K, C2088S, C2088T, C2088L, C2088V, K2095E, K2095N, G2096A, G2096S, N2097D, N2097S, N2097R, N2097K, N2097E, N2097H, N2097A, N2097G, N2097C, N2097P, N2097L, N2097I, N2097M, N2097F, N2097W, N2097Q, N2097T, N2097Y, V2098A, V2098H, V2098P, V2098S, V2098G, V2098C, E2112G, E2112V, D2127N, I2149K, I2149V, E2150K, A2151T, K2154R, Q2155L, L2159P, E2169D, A2178I, A2179T, V2182A, F2196Y, H2223R, A2226S, K2232N, A2241T, F2253I, V2254I, N2262K, K2264R, I2267V, S2276F, S2276P, S2280P, S2297C, L2299I, K2302Q, and K2317T of SEQ ID NO: 2.
6. A plant or plant cell having a mutation in an ACCase gene such that the mutated gene encodes an ACCase protein comprising a substitution at an amino acid position corresponding to D2078 of SEQ ID NO: 2.
7. A plant or plant cell having a mutation in an ACCase gene such that the mutated gene encodes an ACCase protein comprising an amino acid substitution at a position corresponding to D2078 of SEQ ID NO: 2, wherein the substitution is selected from the group consisting of D2078G, D2078K, D2078T, and D2078V.
8. A plant or plant cell having a mutation in an ACCase gene such that the mutated gene encodes an ACCase protein comprising a substitution corresponding to D2078G of SEQ ID NO: 2.
9. A plant or plant cell having a mutation in an ACCase gene such that the mutated gene encodes an ACCase protein comprising a substitution at an amino acid position corresponding to P2083 of SEQ ID NO: 2.
10. A plant or plant cell having a mutation in an ACCase gene such that the mutated gene encodes an ACCase protein comprising an amino acid substitution at a position corresponding to P2083 of SEQ ID NO:2, wherein the substitution is selected from the group consisting of P2083T, P2083S, P2083N, and P2083Q.
11. A plant or plant cell having a mutation in an ACCase gene such that the mutated gene encodes an ACCase protein comprising a substitution corresponding to P2083T of SEQ ID NO: 2.
12. A plant or plant cell having a mutation in an ACCase gene such that the mutated gene encodes an ACCase protein comprising one or more amino acid substitutions corresponding to those selected from the group consisting of D2078G and P2083T of SEQ ID NO: 2.
13. The plant or plant cell of any of the preceding embodiments, wherein the plant or plant cell belongs to the genus Oryza.
14. An Oryza plant or plant cell having a mutation in an ACCase gene such that the mutated gene encodes an ACCase protein comprising a substitution at an amino acid position corresponding to D2089 of SEQ ID NO: 4.
15. An Oryza plant or plant cell having a mutation in an ACCase gene such that the mutated gene encodes an ACCase protein comprising an amino acid substitution at a position corresponding to D2089 of SEQ ID NO: 4, wherein the substitution is selected from the group consisting of D2089G, D2089K, D2089T, and D2089V.
16. An Oryza plant or plant cell having a mutation in an ACCase gene such that the mutated gene encodes an ACCase protein comprising a substitution corresponding to D2089G of SEQ ID NO: 4.
17. An Oryza plant or plant cell having a mutation in an ACCase gene such that the mutated gene encodes an ACCase protein comprising a substitution at an amino acid position corresponding to P2094 of SEQ ID NO: 4.
18. An Oryza plant or plant cell having a mutation in an ACCase gene such that the mutated gene encodes an ACCase protein comprising an amino acid substitution at a position corresponding to P2094 of SEQ ID NO: 4, wherein the substitution is selected from the group consisting of P2094T, P2094S, P2094N, and P2094Q.
19. A plant or plant cell having a mutation in an ACCase gene such that the mutated gene encodes an ACCase protein comprising a substitution corresponding to P2094T of SEQ ID NO: 4.
20. A plant or plant cell having a mutation in an ACCase gene such that the mutated gene encodes an ACCase protein comprising one or more amino acid substitutions corresponding to those selected from the group consisting of D2089G and P2094T of SEQ ID NO: 4.
21. An Oryza plant or plant cell having a mutation in an ACCase gene such that the mutated gene encodes an ACCase protein comprising a substitution at an amino acid position corresponding to D2089 of SEQ ID NO: 954.
22. An Oryza plant or plant cell having a mutation in an ACCase gene such that the mutated gene encodes an ACCase protein comprising an amino acid substitution at a position corresponding to D2089 of SEQ ID NO: 954, wherein the substitution is selected from the group consisting of D2089G, D2089K, D2089T, and D2089V.
23. An Oryza plant or plant cell having a mutation in an ACCase gene such that the mutated gene encodes an ACCase protein comprising a substitution corresponding to D2089G of SEQ ID NO: 954.
24. An Oryza plant or plant cell having a mutation in an ACCase gene such that the mutated gene encodes an ACCase protein comprising a substitution at an amino acid position corresponding to P2094 of SEQ ID NO: 954.
25. An Oryza plant or plant cell having a mutation in an ACCase gene such that the mutated gene encodes an ACCase protein comprising an amino acid substitution at a position corresponding to P2094 of SEQ ID NO: 954, wherein the substitution is selected from the group consisting of P2094T, P2094S, P2094N, and P2094Q.
26. A plant or plant cell having a mutation in an ACCase gene such that the mutated gene encodes an ACCase protein comprising a substitution corresponding to P2094T of SEQ ID NO: 954.
27. A plant or plant cell having a mutation in an ACCase gene such that the mutated gene encodes an ACCase protein comprising one or more amino acid substitutions corresponding to those selected from the group consisting of D2089G and P2094T of SEQ ID NO: 954.
28. An Oryza plant or plant cell having a mutation in an ACCase gene such that the mutated gene encodes an ACCase protein comprising a substitution at an amino acid position corresponding to D2076 of SEQ ID NO: 6.
29. An Oryza plant or plant cell having a mutation in an ACCase gene such that the mutated gene encodes an ACCase protein comprising an amino acid substitution at a position corresponding to D2076 of SEQ ID NO: 6, wherein the substitution is selected from the group consisting of D2076G, D2076K, D2076T, and D2076V.
30 An Oryza plant or plant cell having a mutation in an ACCase gene such that the mutated gene encodes an ACCase protein comprising a substitution corresponding to D2076G of SEQ ID NO: 6.
31. An Oryza plant or plant cell having a mutation in an ACCase gene such that the mutated gene encodes an ACCase protein comprising a substitution at an amino acid position corresponding to P2081 of SEQ ID NO: 6.
32. An Oryza plant or plant cell having a mutation in an ACCase gene such that the mutated gene encodes an ACCase protein comprising an amino acid substitution at a position corresponding to P2081 of SEQ ID NO: 6, wherein the substitution is selected from the group consisting of P2081T, P2081S, P2081N, and P2081Q.
33. A plant or plant cell having a mutation in an ACCase gene such that the mutated gene encodes an ACCase protein comprising a substitution corresponding to P2081T of SEQ ID NO: 6.
34. A plant or plant cell having a mutation in an ACCase gene such that the mutated gene encodes an ACCase protein comprising one or more amino acid substitutions corresponding to those selected from the group consisting of D2076G and P2081T of SEQ ID NO: 6.
35. A plant or plant cell having a mutation in an ACCase gene such that the mutated gene encodes an ACCase protein comprising one or more amino acid substitutions corresponding to those selected from the group consisting of C2088M and N2097S of SEQ ID NO: 2.
36. A plant or plant cell having a mutation in an ACCase gene such that the mutated gene encodes an ACCase protein comprising one or more amino acid substitutions corresponding to those selected from the group consisting of S1784G, S1784N, and A1785G of SEQ ID NO: 2.
37. A plant or plant cell having a mutation in an ACCase gene such that the mutated gene encodes an ACCase protein comprising one or more amino acid substitutions corresponding to those selected from the group consisting of N2097D, N2097S, V2098A, and V2098G of SEQ ID NO: 2.
38. A plant or plant cell having a mutation in an ACCase gene such that the mutated gene encodes an ACCase protein comprising a substitution corresponding to V1529M of SEQ ID NO: 2.
39. A plant or plant cell having a mutation in an ACCase gene such that the mutated gene encodes an ACCase protein comprising a substitution corresponding to L1543F of SEQ ID NO: 2.
40. A plant or plant cell having a mutation in an ACCase gene such that the mutated gene encodes an ACCase protein comprising a substitution corresponding to L1543V of SEQ ID NO: 2.
41. A plant or plant cell having a mutation in an ACCase gene such that the mutated gene encodes an ACCase protein comprising a substitution corresponding to L1555P of SEQ ID NO: 2.
42. A plant or plant cell having a mutation in an ACCase gene such that the mutated gene encodes an ACCase protein comprising a substitution corresponding to C1575S of SEQ ID NO: 2.
43. A plant or plant cell having a mutation in an ACCase gene such that the mutated gene encodes an ACCase protein comprising a substitution corresponding to S1605Y of SEQ ID NO: 2.
44. A plant or plant cell having a mutation in an ACCase gene such that the mutated gene encodes an ACCase protein comprising a substitution corresponding to L1649M of SEQ ID NO: 2.
45. A plant or plant cell having a mutation in an ACCase gene such that the mutated gene encodes an ACCase protein comprising a substitution corresponding to R1672I of SEQ ID NO: 2.
46. A plant or plant cell having a mutation in an ACCase gene such that the mutated gene encodes an ACCase protein comprising a substitution corresponding to D1681E of SEQ ID NO: 2.
47. A plant or plant cell having a mutation in an ACCase gene such that the mutated gene encodes an ACCase protein comprising a substitution corresponding to D1681G of SEQ ID NO: 2.
48. A plant or plant cell having a mutation in an ACCase gene such that the mutated gene encodes an ACCase protein comprising a substitution corresponding to F1683L of SEQ ID NO: 2.
49. A plant or plant cell having a mutation in an ACCase gene such that the mutated gene encodes an ACCase protein comprising a substitution corresponding to V1715A of SEQ ID NO: 2.
50. A plant or plant cell having a mutation in an ACCase gene such that the mutated gene encodes an ACCase protein comprising a substitution corresponding to D1725E of SEQ ID NO: 2.
51. A plant or plant cell having a mutation in an ACCase gene such that the mutated gene encodes an ACCase protein comprising a substitution corresponding to A1752T of SEQ ID NO: 2.
52. A plant or plant cell having a mutation in an ACCase gene such that the mutated gene encodes an ACCase protein comprising a substitution corresponding to I1766V of SEQ ID NO: 2.
53. A plant or plant cell having a mutation in an ACCase gene such that the mutated gene encodes an ACCase protein comprising a substitution corresponding to K1772E of SEQ ID NO: 2.
54. A plant or plant cell having a mutation in an ACCase gene such that the mutated gene encodes an ACCase protein comprising a substitution corresponding to I1781L of SEQ ID NO: 2.
55. A plant or plant cell having a mutation in an ACCase gene such that the mutated gene encodes an ACCase protein comprising a substitution corresponding to I1781T of SEQ ID NO: 2.
56. A plant or plant cell having a mutation in an ACCase gene such that the mutated gene encodes an ACCase protein comprising a substitution corresponding to I1781V of SEQ ID NO: 2.
57. A plant or plant cell having a mutation in an ACCase gene such that the mutated gene encodes an ACCase protein comprising a substitution corresponding to S1784G of SEQ ID NO: 2.
58. A plant or plant cell having a mutation in an ACCase gene such that the mutated gene encodes an ACCase protein comprising a substitution corresponding to S1784N of SEQ ID NO: 2.
59. A plant or plant cell having a mutation in an ACCase gene such that the mutated gene encodes an ACCase protein comprising a substitution corresponding to A1785G of SEQ ID NO: 2.
60. A plant or plant cell having a mutation in an ACCase gene such that the mutated gene encodes an ACCase protein comprising a substitution corresponding to L1818H of SEQ ID NO: 2.
61. A plant or plant cell having a mutation in an ACCase gene such that the mutated gene encodes an ACCase protein comprising a substitution corresponding to L1818I of SEQ ID NO: 2.
62. A plant or plant cell having a mutation in an ACCase gene such that the mutated gene encodes an ACCase protein comprising a substitution corresponding to K1840M of SEQ ID NO: 2.
63. A plant or plant cell having a mutation in an ACCase gene such that the mutated gene encodes an ACCase protein comprising a substitution corresponding to K1840R of SEQ ID NO: 2.
64. A plant or plant cell having a mutation in an ACCase gene such that the mutated gene encodes an ACCase protein comprising a substitution corresponding to K1930N of SEQ ID NO: 2.
65. A plant or plant cell having a mutation in an ACCase gene such that the mutated gene encodes an ACCase protein comprising a substitution corresponding to E1946V of SEQ ID NO: 2.
66. A plant or plant cell having a mutation in an ACCase gene such that the mutated gene encodes an ACCase protein comprising a substitution corresponding to A1957T of SEQ ID NO: 2.
67. A plant or plant cell having a mutation in an ACCase gene such that the mutated gene encodes an ACCase protein comprising a substitution corresponding to V1966L of SEQ ID NO: 2.
68. A plant or plant cell having a mutation in an ACCase gene such that the mutated gene encodes an ACCase protein comprising a substitution corresponding to I1967V of SEQ ID NO: 2.
69. A plant or plant cell having a mutation in an ACCase gene such that the mutated gene encodes an ACCase protein comprising a substitution corresponding to M1974T of SEQ ID NO: 2.
70. A plant or plant cell having a mutation in an ACCase gene such that the mutated gene encodes an ACCase protein comprising a substitution corresponding to M1975T of SEQ ID NO: 2.
71. A plant or plant cell having a mutation in an ACCase gene such that the mutated gene encodes an ACCase protein comprising a substitution corresponding to V1992D of SEQ ID NO: 2.
72. A plant or plant cell having a mutation in an ACCase gene such that the mutated gene encodes an ACCase protein comprising a substitution corresponding to V20491 of SEQ ID NO: 2.
73. A plant or plant cell having a mutation in an ACCase gene such that the mutated gene encodes an ACCase protein comprising a substitution corresponding to A2059V of SEQ ID NO: 2.
74. A plant or plant cell having a mutation in an ACCase gene such that the mutated gene encodes an ACCase protein comprising a substitution corresponding to A2067S of SEQ ID NO: 2.
75. A plant or plant cell having a mutation in an ACCase gene such that the mutated gene encodes an ACCase protein comprising a substitution corresponding to V2075I of SEQ ID NO: 2.
76. A plant or plant cell having a mutation in an ACCase gene such that the mutated gene encodes an ACCase protein comprising a substitution corresponding to V2075L of SEQ ID NO: 2.
77.
78. A plant or plant cell having a mutation in an ACCase gene such that the mutated gene encodes an ACCase protein comprising a substitution corresponding to R2085S of SEQ ID NO: 2.
79. A plant or plant cell having a mutation in an ACCase gene such that the mutated gene encodes an ACCase protein comprising a substitution corresponding to C2088M of SEQ ID NO: 2.
80. A plant or plant cell having a mutation in an ACCase gene such that the mutated gene encodes an ACCase protein comprising a substitution corresponding to C2088N of SEQ ID NO: 2.
81. A plant or plant cell having a mutation in an ACCase gene such that the mutated gene encodes an ACCase protein comprising a substitution corresponding to C2088R of SEQ ID NO: 2.
82. A plant or plant cell having a mutation in an ACCase gene such that the mutated gene encodes an ACCase protein comprising a substitution corresponding to N2097D of SEQ ID NO: 2.
83. A plant or plant cell having a mutation in an ACCase gene such that the mutated gene encodes an ACCase protein comprising a substitution corresponding to N2097S of SEQ ID NO: 2.
84. A plant or plant cell having a mutation in an ACCase gene such that the mutated gene encodes an ACCase protein comprising a substitution corresponding to V2098A of SEQ ID NO: 2.
85. A plant or plant cell having a mutation in an ACCase gene such that the mutated gene encodes an ACCase protein comprising a substitution corresponding to V2098G of SEQ ID NO: 2.
86. A plant or plant cell having a mutation in an ACCase gene such that the mutated gene encodes an ACCase protein comprising a substitution corresponding to E2112V of SEQ ID NO: 2.
87. A plant or plant cell having a mutation in an ACCase gene such that the mutated gene encodes an ACCase protein comprising a substitution corresponding to D2127N of SEQ ID NO: 2.
88. A plant or plant cell having a mutation in an ACCase gene such that the mutated gene encodes an ACCase protein comprising a substitution corresponding to I2149K of SEQ ID NO: 2.
89. A plant or plant cell having a mutation in an ACCase gene such that the mutated gene encodes an ACCase protein comprising a substitution corresponding to E2150K of SEQ ID NO: 2.
90. A plant or plant cell having a mutation in an ACCase gene such that the mutated gene encodes an ACCase protein comprising a substitution corresponding to A2151T of SEQ ID NO: 2.
91. A plant or plant cell having a mutation in an ACCase gene such that the mutated gene encodes an ACCase protein comprising a substitution corresponding to K2154R of SEQ ID NO: 2.
92. A plant or plant cell having a mutation in an ACCase gene such that the mutated gene encodes an ACCase protein comprising a substitution corresponding to Q2155L of SEQ ID NO: 2.
93. A plant or plant cell having a mutation in an ACCase gene such that the mutated gene encodes an ACCase protein comprising a substitution corresponding to L2159P of SEQ ID NO: 2.
94. A plant or plant cell having a mutation in an ACCase gene such that the mutated gene encodes an ACCase protein comprising a substitution corresponding to I2267V of SEQ ID NO: 2.
95. A plant or plant cell having a mutation in an ACCase gene such that the mutated gene encodes an ACCase protein comprising a substitution corresponding to E2169D of SEQ ID NO: 2.
96. A plant or plant cell having a mutation in an ACCase gene such that the mutated gene encodes an ACCase protein comprising a substitution corresponding to A21781 of SEQ ID NO: 2.
97. A plant or plant cell having a mutation in an ACCase gene such that the mutated gene encodes an ACCase protein comprising a substitution corresponding to A2179T of SEQ ID NO: 2.
98. A plant or plant cell having a mutation in an ACCase gene such that the mutated gene encodes an ACCase protein comprising a substitution corresponding to V2182A of SEQ ID NO: 2.
99. A plant or plant cell having a mutation in an ACCase gene such that the mutated gene encodes an ACCase protein comprising a substitution corresponding to F2196Y of SEQ ID NO: 2.
100. A plant or plant cell having a mutation in an ACCase gene such that the mutated gene encodes an ACCase protein comprising a substitution corresponding to H2223R of SEQ ID NO: 2.
101. A plant or plant cell having a mutation in an ACCase gene such that the mutated gene encodes an ACCase protein comprising a substitution corresponding to A2226S of SEQ ID NO: 2.
102. A plant or plant cell having a mutation in an ACCase gene such that the mutated gene encodes an ACCase protein comprising a substitution corresponding to K2232N of SEQ ID NO: 2.
103. A plant or plant cell having a mutation in an ACCase gene such that the mutated gene encodes an ACCase protein comprising a substitution corresponding to F2253I of SEQ ID NO: 2.
104. A plant or plant cell having a mutation in an ACCase gene such that the mutated gene encodes an ACCase protein comprising a substitution corresponding to V2254I of SEQ ID NO: 2.
105. A plant or plant cell having a mutation in an ACCase gene such that the mutated gene encodes an ACCase protein comprising a substitution corresponding to N2262K of SEQ ID NO: 2.
106. A plant or plant cell having a mutation in an ACCase gene such that the mutated gene encodes an ACCase protein comprising a substitution corresponding to S2276F of SEQ ID NO: 2.
107. A plant or plant cell having a mutation in an ACCase gene such that the mutated gene encodes an ACCase protein comprising a substitution corresponding to S2276P of SEQ ID NO: 2.
108. A plant or plant cell having a mutation in an ACCase gene such that the mutated gene encodes an ACCase protein comprising a substitution corresponding to S2280P of SEQ ID NO: 2.
109. A plant or plant cell having a mutation in an ACCase gene such that the mutated gene encodes an ACCase protein comprising a substitution corresponding to S2297C of SEQ ID NO: 2.
110. A plant or plant cell having a mutation in an ACCase gene such that the mutated gene encodes an ACCase protein comprising a substitution corresponding to L22991 of SEQ ID NO: 2.
111. A plant or plant cell having a mutation in an ACCase gene such that the mutated gene encodes an ACCase protein comprising a substitution corresponding to K2302Q of SEQ ID NO: 2.
112. A plant or plant cell having a mutation in an ACCase gene such that the mutated gene encodes an ACCase protein comprising a substitution corresponding to K2317T of SEQ ID NO: 2.
113. A plant or plant cell having a mutation in an ACCase gene such that the mutated gene encodes an ACCase protein comprising one or more amino acid substitutions that differ from the wild-type ACCase gene at one or more of the following positions: 1460, 1529, 1543, 1555, 1575, 1602, 1603, 1605, 1605-6*, 1607, 1649, 1672, 1681, 1681, 1683, 1715, 1725, 1734, 1738, 1739, 1752, 1756, 1766, 1772, 1780, 1781, 1783, 1784, 1785, 1786, 1792, 1811, 1818, 1824, 1837, 1840, 1864, 1870, 1922, 1924, 1930, 1938, 1946, 1957, 1966, 1967, 1974, 1975, 1985, 1992, 1999, 2004, 2027, 2039, 2041, 2049, 2059, 2065, 2067, 2074, 2075, 2078, 2079, 2080, 2081, 2083, 2085, 2088, 2095, 2096, 2097, 2098, 2112, 2119, 2127, 2134, 2149, 2150, 2151, 2154, 2155, 2159, 2267, 2169, 2178, 2179, 2182, 2196, 2220, 2223, 2226, 2232, 2241, 2244, 2253, 2254, 2257, 2262, 2264, 2267, 2276, 2280, 2297, 2299, 2302, and 2317 of SEQ ID NO: 2.
114. A plant or plant cell having a mutation in an ACCase gene such that the mutated gene encodes an ACCase protein comprising a substitution corresponding to any amino acid other than valine at position 1529 of SEQ ID NO: 2.
115. A plant or plant cell having a mutation in an ACCase gene such that the mutated gene encodes an ACCase protein comprising a substitution corresponding to any amino acid other than leucine at position 1543 of SEQ ID NO: 2.
116. A plant or plant cell having a mutation in an ACCase gene such that the mutated gene encodes an ACCase protein comprising a substitution corresponding to any amino acid other than leucine at position 1555 of SEQ ID NO: 2.
117. A plant or plant cell having a mutation in an ACCase gene such that the mutated gene encodes an ACCase protein comprising a substitution corresponding to any amino acid other than cysteine at position 1575 of SEQ ID NO: 2.
118. A plant or plant cell having a mutation in an ACCase gene such that the mutated gene encodes an ACCase protein comprising a substitution corresponding to any amino acid other than asparagine at position 1602 of SEQ ID NO: 2.
119. A plant or plant cell having a mutation in an ACCase gene such that the mutated gene encodes an ACCase protein comprising a substitution corresponding to any amino acid other than isoleucine at position 1603 of SEQ ID NO: 2.
120. A plant or plant cell having a mutation in an ACCase gene such that the mutated gene encodes an ACCase protein comprising a substitution corresponding to any amino acid other than serine at position 1605 of SEQ ID NO: 2.
121. A plant or plant cell having a mutation in an ACCase gene such that the mutated gene encodes an ACCase protein comprising a substitution corresponding to any amino acid other than glycine at position 1605-6 of SEQ ID NO: 2.
122. A plant or plant cell having a mutation in an ACCase gene such that the mutated gene encodes an ACCase protein comprising a substitution corresponding to any amino acid other than serine at position 1605-6 of SEQ ID NO: 2.
123. A plant or plant cell having a mutation in an ACCase gene such that the mutated gene encodes an ACCase protein comprising a substitution corresponding to any amino acid other than leucine at position 1649 of SEQ ID NO: 2.
124. A plant or plant cell having a mutation in an ACCase gene such that the mutated gene encodes an ACCase protein comprising a substitution corresponding to any amino acid other than arginine at position 1672 of SEQ ID NO: 2.
125. A plant or plant cell having a mutation in an ACCase gene such that the mutated gene encodes an ACCase protein comprising a substitution corresponding to any amino acid other than aspartate at position 1681 of SEQ ID NO: 2.
126. A plant or plant cell having a mutation in an ACCase gene such that the mutated gene encodes an ACCase protein comprising a substitution corresponding to any amino acid other than phenylalanine at position 1683 of SEQ ID NO: 2.
127. A plant or plant cell having a mutation in an ACCase gene such that the mutated gene encodes an ACCase protein comprising a substitution corresponding to any amino acid other than valine at position 1715 of SEQ ID NO: 2.
128. A plant or plant cell having a mutation in an ACCase gene such that the mutated gene encodes an ACCase protein comprising a substitution corresponding to any amino acid other than aspartate at position 1725 of SEQ ID NO: 2.
129. A plant or plant cell having a mutation in an ACCase gene such that the mutated gene encodes an ACCase protein comprising a substitution corresponding to any amino acid other than alanine at position 1752 of SEQ ID NO: 2.
130. A plant or plant cell having a mutation in an ACCase gene such that the mutated gene encodes an ACCase protein comprising a substitution corresponding to any amino acid other than isoleucine at position 1766 of SEQ ID NO: 2.
131. A plant or plant cell having a mutation in an ACCase gene such that the mutated gene encodes an ACCase protein comprising a substitution corresponding to any amino acid other than lysine at position 1772 of SEQ ID NO: 2.
132. A plant or plant cell having a mutation in an ACCase gene such that the mutated gene encodes an ACCase protein comprising a substitution corresponding to any amino acid other than serine at position 1784 of SEQ ID NO: 2.
133. A plant or plant cell having a mutation in an ACCase gene such that the mutated gene encodes an ACCase protein comprising a substitution corresponding to any amino acid other than alanine at position 1785 of SEQ ID NO: 2.
134. A plant or plant cell having a mutation in an ACCase gene such that the mutated gene encodes an ACCase protein comprising a substitution corresponding to any amino acid other than leucine at position 1818 of SEQ ID NO: 2.
135. A plant or plant cell having a mutation in an ACCase gene such that the mutated gene encodes an ACCase protein comprising a substitution corresponding to any amino acid other than lysine at position 1840 of SEQ ID NO: 2.
136. A plant or plant cell having a mutation in an ACCase gene such that the mutated gene encodes an ACCase protein comprising a substitution corresponding to any amino acid other than isoleucine at position 1924 of SEQ ID NO: 2.
137. A plant or plant cell having a mutation in an ACCase gene such that the mutated gene encodes an ACCase protein comprising a substitution corresponding to any amino acid other than lysine at position 1930 of SEQ ID NO: 2.
138. A plant or plant cell having a mutation in an ACCase gene such that the mutated gene encodes an ACCase protein comprising a substitution corresponding to any amino acid other than alanine at position 1957 of SEQ ID NO: 2.
139. A plant or plant cell having a mutation in an ACCase gene such that the mutated gene encodes an ACCase protein comprising a substitution corresponding to any amino acid other than valine at position 1966 of SEQ ID NO: 2.
140. A plant or plant cell having a mutation in an ACCase gene such that the mutated gene encodes an ACCase protein comprising a substitution corresponding to any amino acid other than alanine at position 1967 of SEQ ID NO: 2.
141. A plant or plant cell having a mutation in an ACCase gene such that the mutated gene encodes an ACCase protein comprising a substitution corresponding to any amino acid other than methionine at position 1974 of SEQ ID NO: 2.
142. A plant or plant cell having a mutation in an ACCase gene such that the mutated gene encodes an ACCase protein comprising a substitution corresponding to any amino acid other than methionine at position 1975 of SEQ ID NO: 2.
143. A plant or plant cell having a mutation in an ACCase gene such that the mutated gene encodes an ACCase protein comprising a substitution corresponding to any amino acid other than proline at position 1985 of SEQ ID NO: 2.
144. A plant or plant cell having a mutation in an ACCase gene such that the mutated gene encodes an ACCase protein comprising a substitution corresponding to any amino acid other than valine at position 1992 of SEQ ID NO: 2.
145. A plant or plant cell having a mutation in an ACCase gene such that the mutated gene encodes an ACCase protein comprising a substitution corresponding to any amino acid other than lysine at position 2065 of SEQ ID NO: 2.
146. A plant or plant cell having a mutation in an ACCase gene such that the mutated gene encodes an ACCase protein comprising a substitution corresponding to any amino acid other than alanine at position 2067 of SEQ ID NO: 2.
147. A plant or plant cell having a mutation in an ACCase gene such that the mutated gene encodes an ACCase protein comprising a substitution corresponding to any amino acid other than aspartate at position 2078 of SEQ ID NO: 2.
148. A plant or plant cell having a mutation in an ACCase gene such that the mutated gene encodes an ACCase protein comprising a substitution corresponding to any amino acid other than proline at position 2083 of SEQ ID NO: 2.
149. A plant or plant cell having a mutation in an ACCase gene such that the mutated gene encodes an ACCase protein comprising a substitution corresponding to any amino acid other than arginine at position 2085 of SEQ ID NO: 2.
150. A plant or plant cell having a mutation in an ACCase gene such that the mutated gene encodes an ACCase protein comprising a substitution corresponding to any amino acid other than glutamate at position 2112 of SEQ ID NO: 2.
151. A plant or plant cell having a mutation in an ACCase gene such that the mutated gene encodes an ACCase protein comprising a substitution corresponding to any amino acid other than glycine at position 2119 of SEQ ID NO: 2.
152. A plant or plant cell having a mutation in an ACCase gene such that the mutated gene encodes an ACCase protein comprising a substitution corresponding to any amino acid other than aspartate at position 2127 of SEQ ID NO: 2.
153. A plant or plant cell having a mutation in an ACCase gene such that the mutated gene encodes an ACCase protein comprising a substitution corresponding to any amino acid other than glycine at position 2134 of SEQ ID NO: 2.
154. A plant or plant cell having a mutation in an ACCase gene such that the mutated gene encodes an ACCase protein comprising a substitution corresponding to any amino acid other than isoleucine at position 2149 of SEQ ID NO: 2.
155. A plant or plant cell having a mutation in an ACCase gene such that the mutated gene encodes an ACCase protein comprising a substitution corresponding to any amino acid other than glutamate at position 2150 of SEQ ID NO: 2.
156. A plant or plant cell having a mutation in an ACCase gene such that the mutated gene encodes an ACCase protein comprising a substitution corresponding to any amino acid other than alanine at position 2151 of SEQ ID NO: 2.
157. A plant or plant cell having a mutation in an ACCase gene such that the mutated gene encodes an ACCase protein comprising a substitution corresponding to any amino acid other than lysine at position 2154 of SEQ ID NO: 2.
158. A plant or plant cell having a mutation in an ACCase gene such that the mutated gene encodes an ACCase protein comprising a substitution corresponding to any amino acid other than glutamine at position 2155 of SEQ ID NO: 2.
159. A plant or plant cell having a mutation in an ACCase gene such that the mutated gene encodes an ACCase protein comprising a substitution corresponding to any amino acid other than leucine at position 2159 of SEQ ID NO: 2.
160. A plant or plant cell having a mutation in an ACCase gene such that the mutated gene encodes an ACCase protein comprising a substitution corresponding to any amino acid other than isoleucine at position 2267 of SEQ ID NO: 2.
161. A plant or plant cell having a mutation in an ACCase gene such that the mutated gene encodes an ACCase protein comprising a substitution corresponding to any amino acid other than glutamate at position 2169 of SEQ ID NO: 2.
162. A plant or plant cell having a mutation in an ACCase gene such that the mutated gene encodes an ACCase protein comprising a substitution corresponding to any amino acid other than alanine at position 2178 of SEQ ID NO: 2.
163. A plant or plant cell having a mutation in an ACCase gene such that the mutated gene encodes an ACCase protein comprising a substitution corresponding to any amino acid other than alanine at position 2179 of SEQ ID NO: 2.
164. A plant or plant cell having a mutation in an ACCase gene such that the mutated gene encodes an ACCase protein comprising a substitution corresponding to any amino acid other than valine at position 2182 of SEQ ID NO: 2.
165. A plant or plant cell having a mutation in an ACCase gene such that the mutated gene encodes an ACCase protein comprising a substitution corresponding to any amino acid other than phenylalanine at position 2196 of SEQ ID NO: 2.
166. A plant or plant cell having a mutation in an ACCase gene such that the mutated gene encodes an ACCase protein comprising a substitution corresponding to any amino acid other than lysine at position 2220 of SEQ ID NO: 2.
167. A plant or plant cell having a mutation in an ACCase gene such that the mutated gene encodes an ACCase protein comprising a substitution corresponding to any amino acid other than histidine at position 2223 of SEQ ID NO: 2.
168. A plant or plant cell having a mutation in an ACCase gene such that the mutated gene encodes an ACCase protein comprising a substitution corresponding to any amino acid other than alanine at position 2226 of SEQ ID NO: 2.
169. A plant or plant cell having a mutation in an ACCase gene such that the mutated gene encodes an ACCase protein comprising a substitution corresponding to any amino acid other than lysine at position 2232 of SEQ ID NO: 2.
170. A plant or plant cell having a mutation in an ACCase gene such that the mutated gene encodes an ACCase protein comprising a substitution corresponding to any amino acid other than serine at position 2244 of SEQ ID NO: 2.
171. A plant or plant cell having a mutation in an ACCase gene such that the mutated gene encodes an ACCase protein comprising a substitution corresponding to any amino acid other than phenylalanine at position 2253 of SEQ ID NO: 2.
172. A plant or plant cell having a mutation in an ACCase gene such that the mutated gene encodes an ACCase protein comprising a substitution corresponding to any amino acid other than valine at position 2254 of SEQ ID NO: 2.
173. A plant or plant cell having a mutation in an ACCase gene such that the mutated gene encodes an ACCase protein comprising a substitution corresponding to any amino acid other than arginine at position 2257 of SEQ ID NO: 2.
174. A plant or plant cell having a mutation in an ACCase gene such that the mutated gene encodes an ACCase protein comprising a substitution corresponding to any amino acid other than asparagine at position 2262 of SEQ ID NO: 2.
175. A plant or plant cell having a mutation in an ACCase gene such that the mutated gene encodes an ACCase protein comprising a substitution corresponding to any amino acid other than serine at position 2276 of SEQ ID NO: 2.
176. A plant or plant cell having a mutation in an ACCase gene such that the mutated gene encodes an ACCase protein comprising a substitution corresponding to any amino acid other than serine at position 2280 of SEQ ID NO: 2.
177. A plant or plant cell having a mutation in an ACCase gene such that the mutated gene encodes an ACCase protein comprising a substitution corresponding to any amino acid other than serine at position 2297 of SEQ ID NO: 2.
178. A plant or plant cell having a mutation in an ACCase gene such that the mutated gene encodes an ACCase protein comprising a substitution corresponding to any amino acid other than leucine at position 2299 of SEQ ID NO: 2.
179. A plant or plant cell having a mutation in an ACCase gene such that the mutated gene encodes an ACCase protein comprising a substitution corresponding to any amino acid other than lysine at position 2303 of SEQ ID NO: 2.
180. A plant or plant cell having a mutation in an ACCase gene such that the mutated gene encodes an ACCase protein comprising a substitution corresponding to any amino acid other than lysine at position 2317 of SEQ ID NO: 2.
181. A plant or plant cell having a mutation in an ACCase gene such that the mutated gene encodes an ACCase protein comprising one or more mutations resulting in one or more substitution combinations corresponding to one or more combinations selected from the group consisting of V2075L/N2097S; V2075I/N2097S; D2078G/N2097S; V2098A/N2097S; and V2098G/N2097S of SEQ ID NO: 2.
182. A plant or plant cell having a mutation in an ACCase gene such that the mutated gene encodes an ACCase protein comprising one or more mutations resulting in one or more substitution combinations corresponding to V2075L/N2097S of SEQ ID NO: 2.
183. A plant or plant cell having a mutation in an ACCase gene such that the mutated gene encodes an ACCase protein comprising one or more mutations resulting in one or more substitution combinations corresponding to V2075I/N2097S of SEQ ID NO: 2.
184. A plant or plant cell having a mutation in an ACCase gene such that the mutated gene encodes an ACCase protein comprising one or more mutations resulting in one or more substitution combinations corresponding to D2078G/N2097S of SEQ ID NO: 2.
185. A plant or plant cell having a mutation in an ACCase gene such that the mutated gene encodes an ACCase protein comprising one or more mutations resulting in one or more substitution combinations corresponding to V2098A/N2097S of SEQ ID NO: 2.
186. A plant or plant cell having a mutation in an ACCase gene such that the mutated gene encodes an ACCase protein comprising one or more mutations resulting in one or more substitution combinations corresponding to V2098G/N2097S of SEQ ID NO: 2.
187. A plant or plant cell having a mutation in an ACCase gene such that the mutated gene encodes an ACCase protein comprising an amino acid substitution at a position corresponding to N2097 of SEQ ID NO: 2, wherein the substitution is selected from the group consisting N2097R, N2097K, N2097E, N2097H, N2097D, N2097A, N2097G, N2097C, N2097V, N2097P, N2097L, N2097I, N2097M, N2097F, N2097W, N2097Q, N2097S, N2097T, and N2097Y.
188. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation or mutations as provided herein.
189. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation or mutations as provided in Table 1.
190. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation or mutations as provided in Table 2.
191. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation as provided in Table 3.
192. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation at an amino acid position corresponding to 2075 of SEQ ID NO: 2.
193. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation comprising a substitution corresponding to V2075I of SEQ ID NO: 2.
194. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation comprising a substitution corresponding to V2075L of SEQ ID NO: 2.
195. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation at an amino acid position corresponding to 2078 of SEQ ID NO: 2.
196. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation comprising a substitution corresponding to D2078G of SEQ ID NO: 2.
197. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation at an amino acid position corresponding to 2098 of SEQ ID NO: 2.
198. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation comprising a substitution corresponding to V2098A of SEQ ID NO: 2.
199. A plant or plant cell having a mutation in an ACCase gene such that the mutated gene encodes an ACCase protein comprising a substitution corresponding to N2097 of SEQ ID NO: 2 (such as a substitution as provided herein) in combination with an additional ACCase mutation comprising a substitution corresponding to V2098G of SEQ ID NO: 2.
200. A plant or plant cell having a mutation in an ACCase gene such that the mutated gene encodes an ACCase protein comprising one or more mutations resulting in one or more substitution combinations corresponding to V2098G/A2226S of SEQ ID NO: 2.
201. A plant or plant cell having a mutation in an ACCase gene such that the mutated gene encodes an ACCase protein comprising one or more mutations resulting in one or more substitution combinations corresponding to one or more combinations selected from the group consisting of L1543V/D1681E; L1543F/D1681E; L1543V/D1681G; L1543F/D1681G; A1785G/I1781L; A1785G/I1781T; A1785G/I1781V; A1785G/C2088M; A1785G/C2088N; A1785G/C2088R; L1818H/S1784N; L1818H/S1784G; L1818I/S1784N; L1818I/S1784G; V1529M/L1818H; V1529M/L1818I; K1840R/L1818H; K1840R/L1818I; K1840M/L1818H; K1840M/L1818I; N2097S/D2078G; N2097D/D2078G; N2097S/V2075L; N2097D/V2075L; N2097S/V2075I; N2097D/V2075I; N2097S/V2098A; N2097D/V2098A; N2097S/V2098G; N2097D/V2098G; R1672I/D2078G; M1974T/D2078G; D2078G/P2083T; D2078G/C2088M; D2078G/C2088N; D2078G/C2088R; I1781L/V2075I; I1781L/V2075L; I1781T/V2075I; I1781T/V2075L; I1781V/V2075I; I1781V/V2075L; of SEQ ID NO: 2.
202. A plant or plant cell having a mutation in an ACCase gene such that the mutated gene encodes an ACCase protein comprising one or more mutations resulting in one or more substitution combinations corresponding to L1543V/D1681E of SEQ ID NO: 2.
203. A plant or plant cell having a mutation in an ACCase gene such that the mutated gene encodes an ACCase protein comprising one or more mutations resulting in one or more substitution combinations corresponding to L1543F/D1681E of SEQ ID NO: 2.
204. A plant or plant cell having a mutation in an ACCase gene such that the mutated gene encodes an ACCase protein comprising one or more mutations resulting in one or more substitution combinations corresponding to L1543V/D1681G of SEQ ID NO: 2.
205. A plant or plant cell having a mutation in an ACCase gene such that the mutated gene encodes an ACCase protein comprising one or more mutations resulting in one or more substitution combinations corresponding to L1543F/D1681G of SEQ ID NO: 2.
206. A plant or plant cell having a mutation in an ACCase gene such that the mutated gene encodes an ACCase protein comprising one or more mutations resulting in one or more substitution combinations corresponding to one or more combinations selected from the group consisting of S1784G/A1785G; S1784N/A1785G; A1785G/I1781L; A1785G/I1781T; A1785G/I1781V; A1785G/C2088M; A1785G/C2088N; A1785G/C2088R; L1818H/S1784N; L1818H/S1784G; L1818I/S1784N; and L1818I/S1784G of SEQ ID NO: 2.
207. A plant or plant cell having a mutation in an ACCase gene such that the mutated gene encodes an ACCase protein comprising one or more mutations resulting in one or more substitution combinations corresponding to S1784G/A1785G of SEQ ID NO: 2.
208. A plant or plant cell having a mutation in an ACCase gene such that the mutated gene encodes an ACCase protein comprising one or more mutations resulting in one or more substitution combinations corresponding to S1784N/A1785G of SEQ ID NO: 2.
209. A plant or plant cell having a mutation in an ACCase gene such that the mutated gene encodes an ACCase protein comprising one or more mutations resulting in one or more substitution combinations corresponding to A1785G/I1781L of SEQ ID NO: 2.
210. A plant or plant cell having a mutation in an ACCase gene such that the mutated gene encodes an ACCase protein comprising one or more mutations resulting in one or more substitution combinations corresponding to A1785G/I1781T of SEQ ID NO: 2.
211. A plant or plant cell having a mutation in an ACCase gene such that the mutated gene encodes an ACCase protein comprising one or more mutations resulting in one or more substitution combinations corresponding to A1785G/I1781V of SEQ ID NO: 2.
212. A plant or plant cell having a mutation in an ACCase gene such that the mutated gene encodes an ACCase protein comprising one or more mutations resulting in one or more substitution combinations corresponding to A1785G/C2088M of SEQ ID NO: 2.
213. A plant or plant cell having a mutation in an ACCase gene such that the mutated gene encodes an ACCase protein comprising one or more mutations resulting in one or more substitution combinations corresponding to A1785G/C2088N of SEQ ID NO: 2.
214. A plant or plant cell having a mutation in an ACCase gene such that the mutated gene encodes an ACCase protein comprising one or more mutations resulting in one or more substitution combinations corresponding to A1785G/C2088R of SEQ ID NO: 2.
215. A plant or plant cell having a mutation in an ACCase gene such that the mutated gene encodes an ACCase protein comprising one or more mutations resulting in one or more substitution combinations corresponding to L1818H/S1784N of SEQ ID NO: 2.
216. A plant or plant cell having a mutation in an ACCase gene such that the mutated gene encodes an ACCase protein comprising one or more mutations resulting in one or more substitution combinations corresponding to L1818H/S1784G of SEQ ID NO: 2.
217. A plant or plant cell having a mutation in an ACCase gene such that the mutated gene encodes an ACCase protein comprising one or more mutations resulting in one or more substitution combinations corresponding to L1818I/S1784N of SEQ ID NO: 2.
218. A plant or plant cell having a mutation in an ACCase gene such that the mutated gene encodes an ACCase protein comprising one or more mutations resulting in one or more substitution combinations corresponding to L1818I/S1784G of SEQ ID NO: 2.
219. A plant or plant cell having a mutation in an ACCase gene such that the mutated gene encodes an ACCase protein comprising one or more mutations resulting in one or more substitution combinations corresponding to V1529M/L1818H of SEQ ID NO: 2.
220. A plant or plant cell having a mutation in an ACCase gene such that the mutated gene encodes an ACCase protein comprising one or more mutations resulting in one or more substitution combinations corresponding to V1529M/L1818I of SEQ ID NO: 2.
221. A plant or plant cell having a mutation in an ACCase gene such that the mutated gene encodes an ACCase protein comprising one or more mutations resulting in one or more substitution combinations corresponding to K1840R/L1818H of SEQ ID NO: 2.
222. A plant or plant cell having a mutation in an ACCase gene such that the mutated gene encodes an ACCase protein comprising one or more mutations resulting in one or more substitution combinations corresponding to K1840R/L1818I of SEQ ID NO: 2.
223. A plant or plant cell having a mutation in an ACCase gene such that the mutated gene encodes an ACCase protein comprising one or more mutations resulting in one or more substitution combinations corresponding to K1840M/L1818H of SEQ ID NO: 2.
224. A plant or plant cell having a mutation in an ACCase gene such that the mutated gene encodes an ACCase protein comprising one or more mutations resulting in one or more substitution combinations corresponding to K1840M/L1818I of SEQ ID NO: 2.
225. A plant or plant cell having a mutation in an ACCase gene such that the mutated gene encodes an ACCase protein comprising one or more mutations resulting in one or more substitution combinations corresponding to N2097S/D2078G of SEQ ID NO: 2.
226. A plant or plant cell having a mutation in an ACCase gene such that the mutated gene encodes an ACCase protein comprising one or more mutations resulting in one or more substitution combinations corresponding to N2097D/D2078G of SEQ ID NO: 2.
227. A plant or plant cell having a mutation in an ACCase gene such that the mutated gene encodes an ACCase protein comprising one or more mutations resulting in one or more substitution combinations corresponding to N2097S/V2075L of SEQ ID NO: 2.
228. A plant or plant cell having a mutation in an ACCase gene such that the mutated gene encodes an ACCase protein comprising one or more mutations resulting in one or more substitution combinations corresponding to N2097D/V2075L of SEQ ID NO: 2.
229. A plant or plant cell having a mutation in an ACCase gene such that the mutated gene encodes an ACCase protein comprising one or more mutations resulting in one or more substitution combinations corresponding to N2097S/V2075I of SEQ ID NO: 2.
230. A plant or plant cell having a mutation in an ACCase gene such that the mutated gene encodes an ACCase protein comprising one or more mutations resulting in one or more substitution combinations corresponding to N2097D/V2075I of SEQ ID NO: 2.
231. A plant or plant cell having a mutation in an ACCase gene such that the mutated gene encodes an ACCase protein comprising one or more mutations resulting in one or more substitution combinations corresponding to N2097S/V2098A of SEQ ID NO: 2.
232. A plant or plant cell having a mutation in an ACCase gene such that the mutated gene encodes an ACCase protein comprising one or more mutations resulting in one or more substitution combinations corresponding to N2097D/V2098A of SEQ ID NO: 2.
233. A plant or plant cell having a mutation in an ACCase gene such that the mutated gene encodes an ACCase protein comprising one or more mutations resulting in one or more substitution combinations corresponding to N2097S/V2098G of SEQ ID NO: 2.
234. A plant or plant cell having a mutation in an ACCase gene such that the mutated gene encodes an ACCase protein comprising one or more mutations resulting in one or more substitution combinations corresponding to N2097D/V2098G of SEQ ID NO: 2.
235. A plant or plant cell having a mutation in an ACCase gene such that the mutated gene encodes an ACCase protein comprising one or more mutations resulting in one or more substitution combinations corresponding to R1672I/D2078G of SEQ ID NO: 2.
236. A plant or plant cell having a mutation in an ACCase gene such that the mutated gene encodes an ACCase protein comprising one or more mutations resulting in one or more substitution combinations corresponding to M1974T/D2078G of SEQ ID NO: 2.
237. A plant or plant cell having a mutation in an ACCase gene such that the mutated gene encodes an ACCase protein comprising one or more mutations resulting in one or more substitution combinations corresponding to D2078G/P2083T of SEQ ID NO: 2.
238. A plant or plant cell having a mutation in an ACCase gene such that the mutated gene encodes an ACCase protein comprising one or more mutations resulting in one or more substitution combinations corresponding to D2078G/C2088M of SEQ ID NO: 2.
239. A plant or plant cell having a mutation in an ACCase gene such that the mutated gene encodes an ACCase protein comprising one or more mutations resulting in one or more substitution combinations corresponding to D2078G/C2088N of SEQ ID NO: 2.
240. A plant or plant cell having a mutation in an ACCase gene such that the mutated gene encodes an ACCase protein comprising one or more mutations resulting in one or more substitution combinations corresponding to D2078G/C2088R of SEQ ID NO: 2.
241. A plant or plant cell having a mutation in an ACCase gene such that the mutated gene encodes an ACCase protein comprising one or more mutations resulting in one or more substitution combinations corresponding to I1781L/V2075I of SEQ ID NO: 2.
242. A plant or plant cell having a mutation in an ACCase gene such that the mutated gene encodes an ACCase protein comprising one or more mutations resulting in one or more substitution combinations corresponding to I1781L/V2075L of SEQ ID NO: 2.
243. A plant or plant cell having a mutation in an ACCase gene such that the mutated gene encodes an ACCase protein comprising one or more mutations resulting in one or more substitution combinations corresponding to I1781T/V2075I of SEQ ID NO: 2.
244. A plant or plant cell having a mutation in an ACCase gene such that the mutated gene encodes an ACCase protein comprising one or more mutations resulting in one or more substitution combinations corresponding to I1781T/V2075L of SEQ ID NO: 2.
245. A plant or plant cell having a mutation in an ACCase gene such that the mutated gene encodes an ACCase protein comprising one or more mutations resulting in one or more substitution combinations corresponding to I1781V/V2075I of SEQ ID NO: 2.
246. A plant or plant cell having a mutation in an ACCase gene such that the mutated gene encodes an ACCase protein comprising one or more mutations resulting in one or more substitution combinations corresponding to I1781V/V2075L of SEQ ID NO: 2.
247. A plant or plant cell having a mutation in an ACCase gene such that the mutated gene encodes an ACCase protein comprising one or more mutations resulting a substitution combination corresponding to one or more combinations selected from the group consisting of V1529M/L1818H; L1555P/S1605Y/L1985P/D2078G; C1575S/G1605-6A*/V2075L; S1602F/D1725E/I1781T/V2075L; S1603N/L1649M/V1715A/D2078G; S1605Y/V2098A; A1957T/V2075L; V1966L/D2078G/V2182A/A2244T/K2317T; I1967V/V2075L/S2119C; R1672I/M1975T/D2078G; R1672I/D2078G; M1975T/D2078G; F1683L/A1752T/K1930N/V2075L/E2169D; I1766V/D2078G/D2127N; K1772E/S1784N/V1924D; E1946V/D2078G/A2179T/F2253I; M1974T/D2078G/M2257I; M1974T/D2078G; D2078G/M2257I; V1992D/V2075L/F2196Y/L2299I; V2049I/V2075L/A2178I; A2059V/D2078G/S2280P/S2297C; A2059V/D2078G; M2065K/D2078G/R2085S/Q2155L/Q2220L; A2067S/V2075L/L2159P; V2075L/N2097S; D2078G; D2078G/P2083T; D2078G/N2097S/H2223R/S2276F; D2078G/E2112V; D2078G/12149K; D2078G/E2150K/I2267V; D2078G/K2154R/K2232N; D2078G/N2262K; D2078G/S2276P/K2302Q; V2098A/V21341/A2151T/V2254I; V2098G; and V2098G/A2226S of SEQ ID NO: 2.
248. A plant or plant cell having a mutation in an ACCase gene such that the mutated gene encodes an ACCase protein comprising one or more mutations resulting in one or more substitution combinations corresponding to V1529M/L1818H of SEQ ID NO: 2.
249. A plant or plant cell having a mutation in an ACCase gene such that the mutated gene encodes an ACCase protein comprising one or more mutations resulting in one or more substitution combinations corresponding to L1555P/S1605Y/L1985P/D2078G of SEQ ID NO: 2.
250. A plant or plant cell having a mutation in an ACCase gene such that the mutated gene encodes an ACCase protein comprising one or more mutations resulting in one or more substitution combinations corresponding to C1575S/G1605-6A*/V2075L of SEQ ID NO: 2.
251. A plant or plant cell having a mutation in an ACCase gene such that the mutated gene encodes an ACCase protein comprising one or more mutations resulting in one or more substitution combinations corresponding to D1725E/I1781T/V2075L of SEQ ID NO: 2.
252. A plant or plant cell having a mutation in an ACCase gene such that the mutated gene encodes an ACCase protein comprising one or more mutations resulting in one or more substitution combinations corresponding to S1603N/L1649M/V1715A/D2078G of SEQ ID NO: 2.
253. A plant or plant cell having a mutation in an ACCase gene such that the mutated gene encodes an ACCase protein comprising one or more mutations resulting in one or more substitution combinations corresponding to S1605Y/V2098A of SEQ ID NO: 2.
254. A plant or plant cell having a mutation in an ACCase gene such that the mutated gene encodes an ACCase protein comprising one or more mutations resulting in one or more substitution combinations corresponding to A1957T/V2075L of SEQ ID NO: 2.
255. A plant or plant cell having a mutation in an ACCase gene such that the mutated gene encodes an ACCase protein comprising one or more mutations resulting in one or more substitution combinations corresponding to V1966L/D2078G/V2182A/A2244T/K2317T of SEQ ID NO: 2.
256. A plant or plant cell having a mutation in an ACCase gene such that the mutated gene encodes an ACCase protein comprising one or more mutations resulting in one or more substitution combinations corresponding to I1967V/V2075L/S2119C of SEQ ID NO: 2.
257. A plant or plant cell having a mutation in an ACCase gene such that the mutated gene encodes an ACCase protein comprising one or more mutations resulting in one or more substitution combinations corresponding to R1672I/M1975T/D2078G of SEQ ID NO: 2.
258. A plant or plant cell having a mutation in an ACCase gene such that the mutated gene encodes an ACCase protein comprising one or more mutations resulting in one or more substitution combinations corresponding to R1672I/D2078G of SEQ ID NO: 2.
259. A plant or plant cell having a mutation in an ACCase gene such that the mutated gene encodes an ACCase protein comprising one or more mutations resulting in one or more substitution combinations corresponding to M1975T/D2078G of SEQ ID NO: 2.
260. A plant or plant cell having a mutation in an ACCase gene such that the mutated gene encodes an ACCase protein comprising one or more mutations resulting in one or more substitution combinations corresponding to F1683L/A1752T/K1930N/V2075L/E2169D of SEQ ID NO: 2.
261. A plant or plant cell having a mutation in an ACCase gene such that the mutated gene encodes an ACCase protein comprising one or more mutations resulting in one or more substitution combinations corresponding to I1766V/D2078G/D2127N of SEQ ID NO: 2.
262. A plant or plant cell having a mutation in an ACCase gene such that the mutated gene encodes an ACCase protein comprising one or more mutations resulting in one or more substitution combinations corresponding to K1772E/S1784N/V1924D of SEQ ID NO: 2.
263. A plant or plant cell having a mutation in an ACCase gene such that the mutated gene encodes an ACCase protein comprising one or more mutations resulting in one or more substitution combinations corresponding to E1946V/D2078G/A2179T/F2253I of SEQ ID NO: 2.
264. A plant or plant cell having a mutation in an ACCase gene such that the mutated gene encodes an ACCase protein comprising one or more mutations resulting in one or more substitution combinations corresponding to M1974T/D2078G/M2257I of SEQ ID NO: 2.
265. A plant or plant cell having a mutation in an ACCase gene such that the mutated gene encodes an ACCase protein comprising one or more mutations resulting in one or more substitution combinations corresponding to M1974T/D2078G; D2078G/M22571 of SEQ ID NO: 2.
266. A plant or plant cell having a mutation in an ACCase gene such that the mutated gene encodes an ACCase protein comprising one or more mutations resulting in one or more substitution combinations corresponding to V1992D/V2075L/F2196Y/L2299I of SEQ ID NO: 2.
267. A plant or plant cell having a mutation in an ACCase gene such that the mutated gene encodes an ACCase protein comprising one or more mutations resulting in one or more substitution combinations corresponding to V2049I/V2075L/A2178I of SEQ ID NO: 2.
268. A plant or plant cell having a mutation in an ACCase gene such that the mutated gene encodes an ACCase protein comprising one or more mutations resulting in one or more substitution combinations corresponding to A2059V/D2078G/S2280P/S2297C of SEQ ID NO: 2.
269. A plant or plant cell having a mutation in an ACCase gene such that the mutated gene encodes an ACCase protein comprising one or more mutations resulting in one or more substitution combinations corresponding to A2059V/D2078G of SEQ ID NO: 2.
270. A plant or plant cell having a mutation in an ACCase gene such that the mutated gene encodes an ACCase protein comprising one or more mutations resulting in one or more substitution combinations corresponding to M2065K/D2078G/R2085S/Q2155L/Q2220L of SEQ ID NO: 2.
271. A plant or plant cell having a mutation in an ACCase gene such that the mutated gene encodes an ACCase protein comprising one or more mutations resulting in one or more substitution combinations corresponding to A2067S/V2075L/L2159P of SEQ ID NO: 2.
272. A plant or plant cell having a mutation in an ACCase gene such that the mutated gene encodes an ACCase protein comprising one or more mutations resulting in one or more substitution combinations corresponding to V2075L/N2097S of SEQ ID NO: 2.
273. A plant or plant cell having a mutation in an ACCase gene such that the mutated gene encodes an ACCase protein comprising one or more mutations resulting in one or more substitution combinations corresponding to D2078G of SEQ ID NO: 2.
274. A plant or plant cell having a mutation in an ACCase gene such that the mutated gene encodes an ACCase protein comprising one or more mutations resulting in one or more substitution combinations corresponding to D2078G/P2083T of SEQ ID NO: 2.
275. A plant or plant cell having a mutation in an ACCase gene such that the mutated gene encodes an ACCase protein comprising one or more mutations resulting in one or more substitution combinations corresponding to D2078G/N2097S/H2223R/S2276F of SEQ ID NO: 2.
276. A plant or plant cell having a mutation in an ACCase gene such that the mutated gene encodes an ACCase protein comprising one or more mutations resulting in one or more substitution combinations corresponding to D2078G/E2112V of SEQ ID NO: 2.
277. A plant or plant cell having a mutation in an ACCase gene such that the mutated gene encodes an ACCase protein comprising one or more mutations resulting in one or more substitution combinations corresponding to D2078G/12149K of SEQ ID NO: 2.
278. A plant or plant cell having a mutation in an ACCase gene such that the mutated gene encodes an ACCase protein comprising one or more mutations resulting in one or more substitution combinations corresponding to D2078G/E2150K/12267V of SEQ ID NO: 2.
279. A plant or plant cell having a mutation in an ACCase gene such that the mutated gene encodes an ACCase protein comprising one or more mutations resulting in one or more substitution combinations corresponding to D2078G/K2154R/K2232N of SEQ ID NO: 2.
280. A plant or plant cell having a mutation in an ACCase gene such that the mutated gene encodes an ACCase protein comprising one or more mutations resulting in one or more substitution combinations corresponding to D2078G/N2262K of SEQ ID NO: 2.
281. A plant or plant cell having a mutation in an ACCase gene such that the mutated gene encodes an ACCase protein comprising one or more mutations resulting in one or more substitution combinations corresponding to D2078G/S2276P/K2302Q of SEQ ID NO: 2.
282. A plant or plant cell having a mutation in an ACCase gene such that the mutated gene encodes an ACCase protein comprising one or more mutations resulting in one or more substitution combinations corresponding to V2098A/V21341/A2151T/V2254I of SEQ ID NO: 2.
283. A plant or plant cell having a mutation in an ACCase gene such that the mutated gene encodes an ACCase protein comprising one or more mutations resulting in one or more substitution combinations corresponding to V2098G of SEQ ID NO: 2.
284. A plant or plant cell having a mutation in an ACCase gene such that the mutated gene encodes an ACCase protein comprising one or more mutations resulting in one or more substitution combinations corresponding to V2098G/A2226S of SEQ ID NO: 2.
285. The plant or plant cell of any of the preceding embodiments wherein such plant or plant cell resistant to one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, or an agronomically acceptable salts or esters thereof.
286. The plant or plant cell of any of the preceding claims wherein such plant or plant cell is resistant to clethodim.
287. The plant or plant cell of any of the preceding claims wherein such plant or plant cell is fertile.
288. The plant or plant cell of any of the preceding embodiments wherein the plant species is selected from the group consisting of canola, sunflower, corn, tobacco, sugar beet, cotton, maize, wheat (including but not limited to Triticum spp., Triticum aestivum, Triticum durum Triticum timopheevii, Triticum monococcum, Triticum spelta, Triticum zhukovskyi and Triticum urartu and hybrids thereof), barley (including but not limited to Hordeum vulgare L., Hordeum comosum, Hordeum depressum, Hordeum intercedens, Hordeum jubatum, Hordeum marinum, Hordeum marinum, Hordeum parodii, Hordeum pusillum, Hordeum secalinum, and Hordeum spontaneum), rice (including but not limited to Oryza sativa subsp. indica, Oryza sativa subsp. japonica, Oryza sativa subsp. javanica, Oryza sativa subsp. glutinosa (glutinous rice), Oryza sativa Aromatica group (e.g., basmati), and Oryza sativa (floating rice group)), alfalfa, sorghum (e.g., Sorghum bicolor), foxtail millet (e.g., Setaria italica), switchgrass (e.g., Panicum virgatum), Kentucky blue-grass (e.g., Poa pratensis), tomato, mango, peach, apple, pear, strawberry, banana, melon, cassava, potato, carrot, lettuce, onion, soy bean, soya spp, sugar cane, pea, chickpea, field pea, fava bean, lentils, turnip, rutabaga, brussels sprouts, lupin, cauliflower, kale, field beans, poplar, pine, eucalyptus, grape, citrus, triticale, alfalfa, rye (including but not limited to Secale sylvestre, Secale strictum, Secale cereale, Secale vavilovii, Secale africanum, Secale ciliatoglume, Secale ancestrale, and Secale montanum), perennial ryegrass (e.g., Lolium perenne), oats (Avena sativa), turf (including but not limited to Turf grass include Zoysia japonica, Agrostris palustris, Poa pratensis, Poa annua, Digitaria sanguinalis, Cyperus rotundus, Kyllinga brevifolia, Cyperus amuricus, Erigeron canadensis, Hydrocotyle sibthorpioides, Kummerowia striata, Euphorbia humifusa, and Viola arvensis) and forage grasses, flax, oilseed rape, cotton, mustard, cucumber, morning glory, balsam, pepper, eggplant, marigold, lotus, cabbage, daisy, carnation, tulip, iris, and lily.
289. The plant or plant cell of any of the preceding embodiments, wherein the plant or plant cell belongs to the genus Oryza.
290. The plant or plant cell of any of the preceding embodiments, wherein said plant or plant cell is transgenic.
291. The plant or plant cell of any of the preceding embodiments, wherein said plant or plant cell is non-transgenic.
292. A method of making a plant or plant cell of any of the preceding embodiments comprising modifying an ACCase gene of a plant or plant cell.
293. A method of making a plant or plant cell of any of the preceding embodiments comprising transfecting a plant cell with DNA expressing an ACCase protein having a change of any of the preceding embodiments.
294. A method making a plant or plant cell of any of the preceding embodiments, said method comprising exposing a plant cell to a DNA cutter and a modified GRON.
295. A method for making a plant or plant cell of any of the preceding embodiments comprising: (1) introducing into plant cells a gene repair oligonucleobase with a targeted mutation in the ACCase gene to produce plant cells with a mutant ACCase gene that expresses a ACCase protein that is mutated at one or more amino acid positions, (2) selecting a plant cell exhibiting improved tolerance to one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, agronomically acceptable salts and esters of any of these herbicides, and combinations thereof as compared to a corresponding wild-type plant cell; and (3) regenerating a non-transgenic herbicide resistant or tolerant plant having a mutated ACCase gene from said selected plant cell.
296. The method of any of the preceding embodiments, further comprising culturing and regeneration methods to obtain a plant that produces seeds, and the production of seeds and additional plants including descendant (progeny) plants that contain the mutated ACCase gene.
297. The method of any of the preceeding embodiments, wherein said plant is fertile.
298. The plant or plant cell of any of the preceding embodiments having the recited mutation or mutation combination and further comprising at least one additional mutation or mutation combination disclosed herein.
299. A method of causing a genetic change in a plant cell, said method comprising exposing said cell to a DNA cutter and a modified GRON encoding a mutation or mutation combination recited in any of the preceding embodiments.
300. A plant cell comprising a DNA cutter and a modified GRON encoding a mutation or mutation combination recited in any of the preceding embodiments.
301. The method or cell of any of the preceding embodiments, wherein said DNA cutter if present, is one or more selected from a CRISPR, a TALEN, a zinc finger, meganuclease, and a DNA-cutting antibiotic.
302. The method or cell of any of the preceding embodiments, wherein said DNA cutter, if present, is a CRISPR or a TALEN.
303. The method or cell of any of the preceding embodiments, wherein said DNA cutter, if present, is a CRISPR.
304. The method or cell of any of the preceding embodiments, wherein said DNA cutter, if present, is a TALEN.
305. The method or cell of any of the preceding embodiments, wherein said DNA cutter, if present, is one or more DNA-cutting antibiotics selected from the group consisting of bleomycin, zeocin, phleomycin, tallysomycin and pepleomycin.
306. The method or cell of any of the preceding embodiments, wherein said DNA cutter, if present, is zeocin.
307. The method or cell of any of the preceding embodiments, wherein said GRON, if present, is single stranded.
308. The method or cell of any of the preceding embodiments, wherein the GRON, if present, is a chemically protected oligonucleotide.
309. The method or cell of any of the preceding embodiments, wherein the GRON, if present, comprises a chemically protected oligonucleotide protected at the 5′ end.
310. The method or cell of any of the preceding embodiments, wherein the GRON, if present, comprises a chemically protected oligonucleotide protected at the 3′ end.
311. The method or cell of any of the preceding embodiments, wherein the GRON, if present, comprises a chemically protected oligonucleotide protected at the 5′ and 3′ ends.
312. The method or cell of any of the preceding embodiments, wherein the GRON, if present, comprises one or more selected from a Cy3 group, a 3PS group, and a 2′-O-methyl group.
313. The method or cell of any of the preceding embodiments, wherein the GRON, if present, has a Cy3 group.
314. The method or cell of any of the preceding embodiments, wherein the GRON, if present, comprises two or more Cy3 groups.
315. The method or cell of any of the preceding embodiments, wherein the GRON, if present, comprises a Cy3 group at the first base on the 5′ end.
316. The method or cell of any of the preceding embodiments, wherein the GRON, if present, comprises a Cy3 group at the first base on the 3′ end.
317. The method or cell of any of the preceding embodiments, wherein the GRON, if present, comprises a 3PS group.
318. The method or cell of any of the preceding embodiments, wherein the GRON, if present, comprises two or more 3PS groups
319. The method or cell of any of the preceding embodiments, wherein the GRON, if present, comprises a 3PS group at the first base on the 5′ end.
320. The method or cell of any of the preceding embodiments, wherein the GRON, if present, comprises a 3PS group at the first base on the 3′ end.
321. The method or cell of any of the preceding embodiments, wherein the GRON, if present, comprises a 2′-O-methyl group.
322. The method or cell of any of the preceding embodiments, wherein the GRON, if present, comprises two or more 2′-O-methyl groups.
323. The method or cell of any of the preceding embodiments, wherein the GRON, if present, comprises a 2′-O-methyl group at the first base on the 5′ end.
324. The method or cell of any of the preceding embodiments, wherein the GRON, if present, has a 2′-O-methyl group at the first base on the 5′ end and does not have any other 2′-O-methyl groups.
325. The method or cell of any of the preceding embodiments, wherein the GRON, if present, comprises a 2′-O-methyl group on each of the first two or more bases at the 5′ end.
326. The method or cell of any of the preceding embodiments, wherein the GRON, if present, comprises a 2′-O-methyl group on each of the first three or more bases at the 5′ end.
327. The method or cell of any of the preceding embodiments, wherein the GRON, if present, comprises a 2′-O-methyl group on each of the first four or more bases at the 5′ end.
328. The method or cell of any of the preceding embodiments, wherein the GRON, if present, comprises a 2′-O-methyl group on each of the first five or more bases at the 5′ end.
329. The method or cell of any of the preceding embodiments, wherein the GRON, if present, comprises a 2′-O-methyl group on each of the first six or more bases at the 5′ end.
330. The method or cell of any of the preceding embodiments, wherein the GRON, if present, comprises a 2′-O-methyl group on each of the first seven or more bases at the 5′ end.
331. The method or cell of any of the preceding embodiments, wherein the GRON, if present, comprises a 2′-O-methyl group on each of the eight four or more bases at the 5′ end.
332. The method or cell of any of the preceding embodiments, wherein the GRON, if present, comprises a 2′-O-methyl group on each of the first nine or more bases at the 5′ end.
333. The method or cell of any of the preceding embodiments, wherein the GRON, if present, comprises a 2′-O-methyl group on each of the first ten or more bases at the 5′ end.
334. The method or cell of any of the preceding embodiments, wherein the GRON, if present, comprises 1, 2, 3, 4, 5, 6, 7, 8, 9, or 10 RNA base at the 5′ end.
335. The method or cell of any of the preceding embodiments, wherein said GRON, if present, has a wobble base pair relative to the target sequence for the genetic change.
336. The method or cell of any of the preceding embodiments, wherein said GRON, if present, is between 15 and 60 nucleotides in length.
337. The method or cell of any of the preceding embodiments, wherein said GRON, if present, is 41 nucleotides in length.
338. The method or cell of any of the preceding embodiments, wherein said GRON, if present, is between 50 and 110 nucleotides in length.
339. The method or cell of any of the preceding embodiments, wherein said GRON, if present, is 101 nucleotides in length.
340. The method or cell of any of the preceding embodiments, wherein said GRON, if present, is between 150 and 210 nucleotides in length.
341. The method or cell of any of the preceding embodiments, wherein said GRON is 201 nucleotides in length.
342. The method or cell of any of the preceding embodiments, wherein said GRON, if present, is between 70 and 210 nucleotides in length.
343. The method or cell of any of the preceding embodiments, wherein said GRON, if present, is longer than 70 nucleotides in length.
344. The method or cell of any of the preceding embodiments, wherein said GRON, if present, is longer than 165 nucleotides in length.
345. The method or cell of any of the preceding embodiments, wherein said GRON, if present, is longer than 175 nucleotides in length.
346. The method or cell of any of the preceding embodiments, wherein said GRON is longer than 185 nucleotides in length.
347. The method or cell of any of the preceding embodiments, wherein said GRON, if present, is longer than 195 nucleotides in length.
348. The method or cell of any of the preceding embodiments, wherein said GRON, if present, is longer than 200 nucleotides in length.
349. The method or cell of any of the preceding embodiments, wherein said GRON, if present, is longer than 210 nucleotides in length.
350. The method or cell of any of the preceding embodiments, wherein said GRON, if present, is longer than 220 nucleotides in length.
351. The method or cell of any of the preceding embodiments, wherein said GRON, if present, is longer than 230 nucleotides in length.
352. The method or cell of any of the preceding embodiments, wherein said GRON, if present, is longer than 240 nucleotides in length.
353. The method or cell of any of the preceding embodiments, wherein said GRON, if present, is longer than 250 nucleotides in length.
354. The method or cell of any of the preceding embodiments, wherein said GRON, if present, is longer than 260 nucleotides in length.
355. The method or cell of any of the preceding embodiments, wherein said GRON, if present, is longer than 270 nucleotides in length.
356. The method or cell of any of the preceding embodiments, wherein said GRON, if present, is longer than 280 nucleotides in length.
357. The method or cell of any of the preceding embodiments, wherein said GRON, if present, is longer than 290 nucleotides in length.
358. The method or cell of any of the preceding embodiments, wherein said GRON, if present, is longer than 300 nucleotides in length.
359. The method or cell of any of the preceding embodiments, wherein said GRON, if present, is longer than 400 nucleotides in length.
360. The method or cell of any of the preceding embodiments, wherein said GRON, if present, is longer than 500 nucleotides in length.
361. The method or cell of any of the preceding embodiments, wherein said GRON, if present, is longer than 600 nucleotides in length.
362. The method or cell of any of the preceding embodiments, wherein said GRON, if present, is longer than 700 nucleotides in length.
363. The method or cell of any of the preceding embodiments, wherein said GRON, if present, is longer than 800 nucleotides in length.
364. The method or cell of any of the preceding embodiments, wherein said GRON, if present, is longer than 900 nucleotides in length.
365. The method or cell of any of the preceding embodiments, wherein said GRON, if present, is longer than 1000 nucleotides in length.
366. The method or cell of any of the preceding embodiments wherein said plant is selected from the group consisting of canola, sunflower, corn, tobacco, sugar beet, cotton, maize, wheat, barley, rice, alfalfa, sorghum, foxtail millet, switchgrass, Kentucky blue-grass, tomato, mango, peach, apple, pear, strawberry, banana, melon, cassava, potato, carrot, lettuce, onion, soy bean, soya spp, sugar cane, pea, chickpea, field pea, fava bean, lentils, turnip, rutabaga, brussels sprouts, lupin, cauliflower, kale, field beans, poplar, pine, eucalyptus, grape, citrus, triticale, alfalfa, rye, perennial ryegrass, oats, turf and forage grasses, flax, oilseed rape, mustard, cucumber, morning glory, balsam, pepper, eggplant, marigold, lotus, cabbage, daisy, carnation, tulip, iris, cassava, and lily.
367. The method or cell of any of the preceding embodiments wherein said plant is corn.
368. The method or cell of any of the preceding embodiments wherein said plant is wheat.
369. The method or cell of any of the preceding embodiments wherein said plant is barley.
370. The method or cell of any of the preceding embodiments wherein said plant is rice.
371. The method or cell of any of the preceding embodiments wherein said plant is sorghum.
372. The method or cell of any of the preceding embodiments wherein said plant is foxtail millet.
373. The method or cell of any of the preceding embodiments wherein said plant is switchgrass.
374. The method or cell of any of the preceding embodiments wherein said plant is Kentucky blue-grass.
375. The method or cell of any of the preceding embodiments wherein said plant is oats.
376. The method or cell of any of the preceding embodiments wherein said plant is banana.
377. The method or cell of any of the preceding embodiments wherein said plant is onion.
378. The method or cell of any of the preceding embodiments wherein said plant is sugar cane.
379. The method or cell of any of the preceding embodiments wherein said plant is tricale.
380. The method or cell of any of the preceding embodiments wherein said plant is rye.
381. The method or cell of any of the preceding embodiments wherein said plant is perennial ryegrass.
382. The method or cell of any of the preceding embodiments wherein said plant is oat.
383. The method or cell of any of the preceding embodiments wherein said plant is turf.
384. The method or cell of any of the preceding embodiments wherein said plant is a forage grass.
385. The method or cell of any of the preceding embodiments wherein said plant is lotus.
386. The method or cell of any of the preceding embodiments wherein said plant is tulip.
387. The method or cell of any of the preceding embodiments wherein said plant is iris.
388. The method or cell of any of the preceding embodiments wherein said plant is lily.
389. The method or cell of any of the preceding embodiments wherein said plant is a nut producing plant.
390. The method or cell of any of the preceding embodiments wherein two or more guide RNAs are used.
391. The method or cell of any of the preceding embodiments wherein each of the more than one guide RNAs is complimentary to a different target for genetic change.
392. The method or cell of any of the preceding embodiments wherein the CRISPR includes a nickase.
393. The method or cell of any of the preceding embodiments wherein the DNA cutter includes two or more nickases.
394. The method or cell of any of the preceding embodiments wherein two or more nickases cuts on opposite strands of the target nucleic acid sequence.
395. The method or cell of any of the preceding embodiments wherein two or more nickases cuts on the same strand of the target nucleic acid sequence.
396. A plant or plant cell expressing an ACCase protein comprising one or more amino acid substitutions corresponding to the group consisting of the mutations listed on Table 1.
397. A plant or plant cell expressing an ACCase protein comprising a substitution corresponding to a change selected from the group consisting of the mutations listed on Table 2.
398. A plant or plant cell expressing an ACCase protein comprising a substitution corresponding to a change selected from the group consisting of the mutations listed on Table 3.
399. A plant or plant cell expressing an ACCase protein comprising one or more amino acid substitutions at one or more amino acid positions corresponding to those selected from the group consisting of N1460, V1529, L1543, L1555, C1575, S1605, G1605-6A*, N1607, L1649, R1672, D1681, F1683, V1715, D1725, R1734, M1738, T1739, A1752, Q1756, 11766, K1772, N1780, 11781, G1783, S1784, A1785, A1786, S1792, I1811, L1818, Q1824, A1837, K1840, V1864, P1870, S1922, K1930, K1938, E1946, A1957, V1966, I1967, M1974, M1975, V1992, W1999, A2004, W2027, E2039, I2041, V2049, A2059, A2067, W2074, V2075, D2078, S2079, K2080, I2081, P2083, R2085, C2088, K2095, G2096, N2097, V2098, E2112, D2127, I2149, E2150, K2154, Q2155, L2159, E2169, A2178, A2179, V2182, F2196, H2223, A2226, K2232, A2241, S2244, F2253, V2254, R2257, N2262, K2264, I2267, S2276, S2280, S2297, L2299, K2302, and K2317 of SEQ ID NO: 2.
400. A plant or plant cell expressing an ACCase protein comprising one or more amino acid substitutions corresponding to those selected from the group consisting of N1460S, V1529M, L1543F, L1543V, L1555P, C1575S, S1605Y, G1605-6A*, N1607S, L1649M, R1672I, D1681E, D1681G, F1683L, V1715A, D1725E, R1734G, M1738L, T1739S, A1752T, Q1756P, Q1756E, I1766V, K1772E, K1772N, N1780D, I1781L, I1781T, I1781V, I1781A, G1783C, S1784G, S1784N, A1785G, A1786P, S1792L, I1811N, L1818H, L1818I, Q1824P, A1837V, K1840M, K1840R, V1864F, P1870R, S1922G, K1930N, K1938R, E1946G, E1946D, E1946V, A1957T, V1966L, 11967V, M1974T, M1975T, V1992D, W1999C, W1999G, W1999L, W1999S, A2004V, W2027C, W2027R, E2039D, E2039G, I2041D, I2041N, I2041V, V2049F, V2049I, V2049L, V2049T, V2049C, V2049A, V2049S, A2059V, A2067S, W2074L, V2075M, V2075G, V2075I, V2075L, D2078G, D2078K, D2078T, D2078V, S2079F, S2079P, K2080E, P2083N, P2083Q, P2083S, P2083T, R2085S, C2088M, C2088N, C2088R, C2088W, C2088F, C2088G, C2088H, C2088K, C2088S, C2088T, C2088L, C2088V, K2095E, K2095N, G2096A, G2096S, N2097D, N2097S, N2097R, N2097K, N2097E, N2097H, N2097A, N2097G, N2097C, N2097P, N2097L, N2097I, N2097M, N2097F, N2097W, N2097Q, N2097T, N2097Y, V2098A, V2098H, V2098P, V2098S, V2098G, V2098C, E2112G, E2112V, D2127N, I2149K, I2149V, E2150K, A2151T, K2154R, Q2155L, L2159P, E2169D, A2178I, A2179T, V2182A, F2196Y, H2223R, A2226S, K2232N, A2241T, F2253I, V2254I, N2262K, K2264R, I2267V, S2276F, S2276P, S2280P, S2297C, L2299I, K2302Q, and K2317T of SEQ ID NO: 2.
401. A plant or plant cell expressing an ACCase protein comprising a substitution at an amino acid position corresponding to D2078 of SEQ ID NO: 2.
402. A plant or plant cell expressing an ACCase protein comprising an amino acid substitution at a position corresponding to D2078 of SEQ ID NO: 2, wherein the substitution is selected from the group consisting of D2078G, D2078K, D2078T, and D2078V.
403. A plant or plant cell expressing an ACCase protein comprising a substitution corresponding to D2078G of SEQ ID NO: 2.
404. A plant or plant cell expressing an ACCase protein comprising a substitution at an amino acid position corresponding to P2083 of SEQ ID NO: 2.
405. A plant or plant cell expressing an ACCase protein comprising an amino acid substitution at a position corresponding to P2083 of SEQ ID NO: 2, wherein the substitution is selected from the group consisting of P2083T, P2083S, P2083N, and P2083Q.
406. A plant or plant cell expressing an ACCase protein comprising a substitution corresponding to P2083T of SEQ ID NO: 2.
407. A plant or plant cell expressing an ACCase protein comprising one or more amino acid substitutions corresponding to those selected from the group consisting of D2078G and P2083T of SEQ ID NO: 2.
408. The plant or plant cell of any of the preceding embodiments, wherein the plant or plant cell belongs to the genus Oryza.
409. An Oryza plant or plant cell expressing an ACCase protein comprising a substitution at an amino acid position corresponding to D2089 of SEQ ID NO: 4.
410. An Oryza plant or plant cell expressing an ACCase protein comprising an amino acid substitution at a position corresponding to D2089 of SEQ ID NO: 4, wherein the substitution is selected from the group consisting of D2089G, D2089K, D2089T, and D2089V.
411. An Oryza plant or plant cell expressing an ACCase protein comprising a substitution corresponding to D2089G of SEQ ID NO: 4.
412. An Oryza plant or plant cell expressing an ACCase protein comprising a substitution at an amino acid position corresponding to P2094 of SEQ ID NO: 4.
413. An Oryza plant or plant cell expressing an ACCase protein comprising an amino acid substitution at a position corresponding to P2094 of SEQ ID NO: 4, wherein the substitution is selected from the group consisting of P2094T, P2094S, P2094N, and P2094Q.
414. A plant or plant cell expressing an ACCase protein comprising a substitution corresponding to P2094T of SEQ ID NO: 4.
415. A plant or plant cell expressing an ACCase protein comprising one or more amino acid substitutions corresponding to those selected from the group consisting of D2089G and P2094T of SEQ ID NO: 4.
416. An Oryza plant or plant cell expressing an ACCase protein comprising a substitution at an amino acid position corresponding to D2089 of SEQ ID NO: 954.
417. An Oryza plant or plant cell expressing an ACCase protein comprising an amino acid substitution at a position corresponding to D2089 of SEQ ID NO: 954, wherein the substitution is selected from the group consisting of D2089G, D2089K, D2089T, and D2089V.
418. An Oryza plant or plant cell expressing an ACCase protein comprising a substitution corresponding to D2089G of SEQ ID NO: 954.
419. An Oryza plant or plant cell expressing an ACCase protein comprising a substitution at an amino acid position corresponding to P2094 of SEQ ID NO: 954.
420. An Oryza plant or plant cell expressing an ACCase protein comprising an amino acid substitution at a position corresponding to P2094 of SEQ ID NO: 954, wherein the substitution is selected from the group consisting of P2094T, P2094S, P2094N, and P2094Q.
421. A plant or plant cell expressing an ACCase protein comprising a substitution corresponding to P2094T of SEQ ID NO: 954.
422. A plant or plant cell expressing an ACCase protein comprising one or more amino acid substitutions corresponding to those selected from the group consisting of D2089G and P2094T of SEQ ID NO: 954.
423. An Oryza plant or plant cell expressing an ACCase protein comprising a substitution at an amino acid position corresponding to D2076 of SEQ ID NO: 6.
424. An Oryza plant or plant cell expressing an ACCase protein comprising an amino acid substitution corresponding to D2076 of SEQ ID NO: 6, wherein the substitution is selected from the group consisting of D2076G, D2076K, D2076T, and D2076V.
425. An Oryza plant or plant cell expressing an ACCase protein comprising a substitution corresponding to D2076G of SEQ ID NO: 6.
426. An Oryza plant or plant cell expressing an ACCase protein comprising a substitution at an amino acid position corresponding to P2081 of SEQ ID NO: 6.
427. An Oryza plant or plant cell expressing an ACCase protein comprising an amino acid substitution corresponding to P2081 of SEQ ID NO: 6, wherein the substitution is selected from the group consisting of P2081T, P2081S, P2081N, and P2081Q.
428. A plant or plant cell expressing an ACCase protein comprising a substitution corresponding to P2081T of SEQ ID NO: 6.
429. A plant or plant cell expressing an ACCase protein comprising one or more amino acid substitutions corresponding to those selected from the group consisting of D2076G and P2081T of SEQ ID NO: 6.
430. A plant or plant cell expressing an ACCase protein comprising one or more amino acid substitutions corresponding to those selected from the group consisting of C2088M and N2097S of SEQ ID NO: 2.
431. A plant or plant cell expressing an ACCase protein comprising one or more amino acid substitutions corresponding to those selected from the group consisting of S1784G, S1784N, and A1785G of SEQ ID NO: 2.
432. A plant or plant cell expressing an ACCase protein comprising one or more amino acid substitutions corresponding to those selected from the group consisting of N2097D, N2097S, V2098A, and V2098G of SEQ ID NO: 2.
433. A plant or plant cell expressing an ACCase protein comprising a substitution corresponding to V1529M of SEQ ID NO: 2.
434. A plant or plant cell expressing an ACCase protein comprising a substitution corresponding to L1543F of SEQ ID NO: 2.
435. A plant or plant cell expressing an ACCase protein comprising a substitution corresponding to L1543V of SEQ ID NO: 2.
436. A plant or plant cell expressing an ACCase protein comprising a substitution corresponding to L1555P of SEQ ID NO: 2.
437. A plant or plant cell expressing an ACCase protein comprising a substitution corresponding to C1575S of SEQ ID NO: 2.
438. A plant or plant cell expressing an ACCase protein comprising a substitution corresponding to S1605Y of SEQ ID NO: 2.
439. A plant or plant cell expressing an ACCase protein comprising a substitution corresponding to L1649M of SEQ ID NO: 2.
440. A plant or plant cell expressing an ACCase protein comprising a substitution corresponding to R1672I of SEQ ID NO: 2.
441. A plant or plant cell expressing an ACCase protein comprising a substitution corresponding to D1681E of SEQ ID NO: 2.
442. A plant or plant cell expressing an ACCase protein comprising a substitution corresponding to D1681G of SEQ ID NO: 2.
443. A plant or plant cell expressing an ACCase protein comprising a substitution corresponding to F1683L of SEQ ID NO: 2.
444. A plant or plant cell expressing an ACCase protein comprising a substitution corresponding to V1715A of SEQ ID NO: 2.
445. A plant or plant cell expressing an ACCase protein comprising a substitution corresponding to D1725E of SEQ ID NO: 2.
446. A plant or plant cell expressing an ACCase protein comprising a substitution corresponding to A1752T of SEQ ID NO: 2.
447. A plant or plant cell expressing an ACCase protein comprising a substitution corresponding to I1766V of SEQ ID NO: 2.
448. A plant or plant cell expressing an ACCase protein comprising a substitution corresponding to K1772E of SEQ ID NO: 2.
449. A plant or plant cell expressing an ACCase protein comprising a substitution corresponding to I1781L of SEQ ID NO: 2.
450. A plant or plant cell expressing an ACCase protein comprising a substitution corresponding to I1781T of SEQ ID NO: 2.
451. A plant or plant cell expressing an ACCase protein comprising a substitution corresponding to I1781V of SEQ ID NO: 2.
452. A plant or plant cell expressing an ACCase protein comprising a substitution corresponding to S1784G of SEQ ID NO: 2.
453. A plant or plant cell expressing an ACCase protein comprising a substitution corresponding to S1784N of SEQ ID NO: 2.
454. A plant or plant cell expressing an ACCase protein comprising a substitution corresponding to A1785G of SEQ ID NO: 2.
455. A plant or plant cell expressing an ACCase protein comprising a substitution corresponding to L1818H of SEQ ID NO: 2.
456. A plant or plant cell expressing an ACCase protein comprising a substitution corresponding to L18181 of SEQ ID NO: 2.
457. A plant or plant cell expressing an ACCase protein comprising a substitution corresponding to K1840M of SEQ ID NO: 2.
458. A plant or plant cell expressing an ACCase protein comprising a substitution corresponding to K1840R of SEQ ID NO: 2.
459. A plant or plant cell expressing an ACCase protein comprising a substitution corresponding to K1930N of SEQ ID NO: 2.
460. A plant or plant cell expressing an ACCase protein comprising a substitution corresponding to E1946V of SEQ ID NO: 2.
461. A plant or plant cell expressing an ACCase protein comprising a substitution corresponding to A1957T of SEQ ID NO: 2.
462. A plant or plant cell expressing an ACCase protein comprising a substitution corresponding to V1966L of SEQ ID NO: 2.
463. A plant or plant cell expressing an ACCase protein comprising a substitution corresponding to 11967V of SEQ ID NO: 2.
464. A plant or plant cell expressing an ACCase protein comprising a substitution corresponding to M1974T of SEQ ID NO: 2.
465. A plant or plant cell expressing an ACCase protein comprising a substitution corresponding to M1975T of SEQ ID NO: 2.
466. A plant or plant cell expressing an ACCase protein comprising a substitution corresponding to V1992D of SEQ ID NO: 2.
467. A plant or plant cell expressing an ACCase protein comprising a substitution corresponding to V20491 of SEQ ID NO: 2.
468. A plant or plant cell expressing an ACCase protein comprising a substitution corresponding to A2059V of SEQ ID NO: 2.
469. A plant or plant cell expressing an ACCase protein comprising a substitution corresponding to A2067S of SEQ ID NO: 2.
470. A plant or plant cell expressing an ACCase protein comprising a substitution corresponding to V20751 of SEQ ID NO: 2.
471. A plant or plant cell expressing an ACCase protein comprising a substitution corresponding to V2075L of SEQ ID NO: 2.
472. A plant or plant cell expressing an ACCase protein comprising a substitution corresponding to R2085S of SEQ ID NO: 2.
473. A plant or plant cell expressing an ACCase protein comprising a substitution corresponding to C2088M of SEQ ID NO: 2.
474. A plant or plant cell expressing an ACCase protein comprising a substitution corresponding to C2088N of SEQ ID NO: 2.
475. A plant or plant cell expressing an ACCase protein comprising a substitution corresponding to C2088R of SEQ ID NO: 2.
476. A plant or plant cell expressing an ACCase protein comprising a substitution corresponding to N2097D of SEQ ID NO: 2.
477. A plant or plant cell expressing an ACCase protein comprising a substitution corresponding to N2097S of SEQ ID NO: 2.
478. A plant or plant cell expressing an ACCase protein comprising a substitution corresponding to V2098A of SEQ ID NO: 2.
479. A plant or plant cell expressing an ACCase protein comprising a substitution corresponding to V2098G of SEQ ID NO: 2.
480. A plant or plant cell expressing an ACCase protein comprising a substitution corresponding to E2112V of SEQ ID NO: 2.
481. A plant or plant cell expressing an ACCase protein comprising a substitution corresponding to D2127N of SEQ ID NO: 2.
482. A plant or plant cell expressing an ACCase protein comprising a substitution corresponding to I2149K of SEQ ID NO: 2.
483. A plant or plant cell expressing an ACCase protein comprising a substitution corresponding to E2150K of SEQ ID NO: 2.
484. A plant or plant cell expressing an ACCase protein comprising a substitution corresponding to A2151T of SEQ ID NO: 2.
485. A plant or plant cell expressing an ACCase protein comprising a substitution corresponding to K2154R of SEQ ID NO: 2.
486. A plant or plant cell expressing an ACCase protein comprising a substitution corresponding to Q2155L of SEQ ID NO: 2.
487. A plant or plant cell expressing an ACCase protein comprising a substitution corresponding to L2159P of SEQ ID NO: 2.
488. A plant or plant cell expressing an ACCase protein comprising a substitution corresponding to I2267V of SEQ ID NO: 2.
489. A plant or plant cell expressing an ACCase protein comprising a substitution corresponding to E2169D of SEQ ID NO: 2.
490. A plant or plant cell expressing an ACCase protein comprising a substitution corresponding to A21781 of SEQ ID NO: 2.
491. A plant or plant cell expressing an ACCase protein comprising a substitution corresponding to A2179T of SEQ ID NO: 2.
492. A plant or plant cell expressing an ACCase protein comprising a substitution corresponding to V2182A of SEQ ID NO: 2.
493. A plant or plant cell expressing an ACCase protein comprising a substitution corresponding to F2196Y of SEQ ID NO: 2.
494. A plant or plant cell expressing an ACCase protein comprising a substitution corresponding to H2223R of SEQ ID NO: 2.
495. A plant or plant cell expressing an ACCase protein comprising a substitution corresponding to A2226S of SEQ ID NO: 2.
496. A plant or plant cell expressing an ACCase protein comprising a substitution corresponding to K2232N of SEQ ID NO: 2.
497. A plant or plant cell expressing an ACCase protein comprising a substitution corresponding to F22531 of SEQ ID NO: 2.
498. A plant or plant cell expressing an ACCase protein comprising a substitution corresponding to V2254I of SEQ ID NO: 2.
499. A plant or plant cell expressing an ACCase protein comprising a substitution corresponding to N2262K of SEQ ID NO: 2.
500. A plant or plant cell expressing an ACCase protein comprising a substitution corresponding to S2276F of SEQ ID NO: 2.
501. A plant or plant cell expressing an ACCase protein comprising a substitution corresponding to S2276P of SEQ ID NO: 2.
502. A plant or plant cell expressing an ACCase protein comprising a substitution corresponding to S2280P of SEQ ID NO: 2.
503. A plant or plant cell expressing an ACCase protein comprising a substitution corresponding to S2297C of SEQ ID NO: 2.
504. A plant or plant cell expressing an ACCase protein comprising a substitution corresponding to L22991 of SEQ ID NO: 2.
505. A plant or plant cell expressing an ACCase protein comprising a substitution corresponding to K2302Q of SEQ ID NO: 2.
506. A plant or plant cell expressing an ACCase protein comprising a substitution corresponding to K2317T of SEQ ID NO: 2.
507. A plant or plant cell expressing an ACCase protein comprising one or more amino acid substitutions that differ from the wild-type ACCase gene at one or more of the following positions: 1460, 1529, 1543, 1555, 1575, 1602, 1603, 1605, 1605-6*, 1607, 1649, 1672, 1681, 1681, 1683, 1715, 1725, 1734, 1738, 1739, 1752, 1756, 1766, 1772, 1780, 1781, 1783, 1784, 1785, 1786, 1792, 1811, 1818, 1824, 1837, 1840, 1864, 1870, 1922, 1924, 1930, 1938, 1946, 1957, 1966, 1967, 1974, 1975, 1985, 1992, 1999, 2004, 2027, 2039, 2041, 2049, 2059, 2065, 2067, 2074, 2075, 2078, 2079, 2080, 2081, 2083, 2085, 2088, 2095, 2096, 2097, 2098, 2112, 2119, 2127, 2134, 2149, 2150, 2151, 2154, 2155, 2159, 2267, 2169, 2178, 2179, 2182, 2196, 2220, 2223, 2226, 2232, 2241, 2244, 2253, 2254, 2257, 2262, 2264, 2267, 2276, 2280, 2297, 2299, 2302, and 2317 of SEQ ID NO: 2.
508. A plant or plant cell expressing an ACCase protein comprising a substitution corresponding to any amino acid other than valine at position 1529 of SEQ ID NO: 2.
509. A plant or plant cell expressing an ACCase protein comprising a substitution corresponding to any amino acid other than leucine at position 1543 of SEQ ID NO: 2.
510. A plant or plant cell expressing an ACCase protein comprising a substitution corresponding to any amino acid other than leucine at position 1555 of SEQ ID NO: 2.
511. A plant or plant cell expressing an ACCase protein comprising a substitution corresponding to any amino acid other than cysteine at position 1575 of SEQ ID NO: 2.
512. A plant or plant cell expressing an ACCase protein comprising a substitution corresponding to any amino acid other than asparagine at position 1602 of SEQ ID NO: 2.
513. A plant or plant cell expressing an ACCase protein comprising a substitution corresponding to any amino acid other than isoleucine at position 1603 of SEQ ID NO: 2.
514. A plant or plant cell expressing an ACCase protein comprising a substitution corresponding to any amino acid other than serine at position 1605 of SEQ ID NO: 2.
515. A plant or plant cell expressing an ACCase protein comprising a substitution corresponding to any amino acid other than glycine at position 1605-6 of SEQ ID NO: 2.
516. A plant or plant cell expressing an ACCase protein comprising a substitution corresponding to any amino acid other than serine at position 1605-6 of SEQ ID NO: 2.
517. A plant or plant cell expressing an ACCase protein comprising a substitution corresponding to any amino acid other than leucine at position 1649 of SEQ ID NO: 2.
518. A plant or plant cell expressing an ACCase protein comprising a substitution corresponding to any amino acid other than arginine at position 1672 of SEQ ID NO: 2.
519. A plant or plant cell expressing an ACCase protein comprising a substitution corresponding to any amino acid other than aspartate at position 1681 of SEQ ID NO: 2.
520. A plant or plant cell expressing an ACCase protein comprising a substitution corresponding to any amino acid other than phenylalanine at position 1683 of SEQ ID NO: 2.
521. A plant or plant cell expressing an ACCase protein comprising a substitution corresponding to any amino acid other than valine at position 1715 of SEQ ID NO: 2.
522. A plant or plant cell expressing an ACCase protein comprising a substitution corresponding to any amino acid other than aspartate at position 1725 of SEQ ID NO: 2.
523. A plant or plant cell expressing an ACCase protein comprising a substitution corresponding to any amino acid other than alanine at position 1752 of SEQ ID NO: 2.
524. A plant or plant cell expressing an ACCase protein comprising a substitution corresponding to any amino acid other than isoleucine at position 1766 of SEQ ID NO: 2.
525. A plant or plant cell expressing an ACCase protein comprising a substitution corresponding to any amino acid other than lysine at position 1772 of SEQ ID NO: 2.
526. A plant or plant cell expressing an ACCase protein comprising a substitution corresponding to any amino acid other than serine at position 1784 of SEQ ID NO: 2.
527. A plant or plant cell expressing an ACCase protein comprising a substitution corresponding to any amino acid other than alanine at position 1785 of SEQ ID NO: 2.
528. A plant or plant cell expressing an ACCase protein comprising a substitution corresponding to any amino acid other than leucine at position 1818 of SEQ ID NO: 2.
529. A plant or plant cell expressing an ACCase protein comprising a substitution corresponding to any amino acid other than lysine at position 1840 of SEQ ID NO: 2.
530. A plant or plant cell expressing an ACCase protein comprising a substitution corresponding to any amino acid other than isoleucine at position 1924 of SEQ ID NO: 2.
531. A plant or plant cell expressing an ACCase protein comprising a substitution corresponding to any amino acid other than lysine at position 1930 of SEQ ID NO: 2.
532. A plant or plant cell expressing an ACCase protein comprising a substitution corresponding to any amino acid other than alanine at position 1957 of SEQ ID NO: 2.
533. A plant or plant cell expressing an ACCase protein comprising a substitution corresponding to any amino acid other than valine at position 1966 of SEQ ID NO: 2.
534. A plant or plant cell expressing an ACCase protein comprising a substitution corresponding to any amino acid other than alanine at position 1967 of SEQ ID NO: 2.
535. A plant or plant cell expressing an ACCase protein comprising a substitution corresponding to any amino acid other than methionine at position 1974 of SEQ ID NO: 2.
536. A plant or plant cell expressing an ACCase protein comprising a substitution corresponding to any amino acid other than methionine at position 1975 of SEQ ID NO: 2.
537. A plant or plant cell expressing an ACCase protein comprising a substitution corresponding to any amino acid other than proline at position 1985 of SEQ ID NO: 2.
538. A plant or plant cell expressing an ACCase protein comprising a substitution corresponding to any amino acid other than valine at position 1992 of SEQ ID NO: 2.
539. A plant or plant cell expressing an ACCase protein comprising a substitution corresponding to any amino acid other than lysine at position 2065 of SEQ ID NO: 2.
540. A plant or plant cell expressing an ACCase protein comprising a substitution corresponding to any amino acid other than alanine at position 2067 of SEQ ID NO: 2.
541. A plant or plant cell expressing an ACCase protein comprising a substitution corresponding to any amino acid other than aspartate at position 2078 of SEQ ID NO: 2.
542. A plant or plant cell expressing an ACCase protein comprising a substitution corresponding to any amino acid other than proline at position 2083 of SEQ ID NO: 2.
543. A plant or plant cell expressing an ACCase protein comprising a substitution corresponding to any amino acid other than arginine at position 2085 of SEQ ID NO: 2.
544. A plant or plant cell expressing an ACCase protein comprising a substitution corresponding to any amino acid other than glutamate at position 2112 of SEQ ID NO: 2.
545. A plant or plant cell expressing an ACCase protein comprising a substitution corresponding to any amino acid other than glycine at position 2119 of SEQ ID NO: 2.
546. A plant or plant cell expressing an ACCase protein comprising a substitution corresponding to any amino acid other than aspartate at position 2127 of SEQ ID NO: 2.
547. A plant or plant cell expressing an ACCase protein comprising a substitution corresponding to any amino acid other than glycine at position 2134 of SEQ ID NO: 2.
548. A plant or plant cell expressing an ACCase protein comprising a substitution corresponding to any amino acid other than isoleucine at position 2149 of SEQ ID NO: 2.
549. A plant or plant cell expressing an ACCase protein comprising a substitution corresponding to any amino acid other than glutamate at position 2150 of SEQ ID NO: 2.
550. A plant or plant cell expressing an ACCase protein comprising a substitution corresponding to any amino acid other than alanine at position 2151 of SEQ ID NO: 2.
551. A plant or plant cell expressing an ACCase protein comprising a substitution corresponding to any amino acid other than lysine at position 2154 of SEQ ID NO: 2.
552. A plant or plant cell expressing an ACCase protein comprising a substitution corresponding to any amino acid other than glutamine at position 2155 of SEQ ID NO: 2.
553. A plant or plant cell expressing an ACCase protein comprising a substitution corresponding to any amino acid other than leucine at position 2159 of SEQ ID NO: 2.
554. A plant or plant cell expressing an ACCase protein comprising a substitution corresponding to any amino acid other than isoleucine at position 2267 of SEQ ID NO: 2.
555. A plant or plant cell expressing an ACCase protein comprising a substitution corresponding to any amino acid other than glutamate at position 2169 of SEQ ID NO: 2.
556. A plant or plant cell expressing an ACCase protein comprising a substitution corresponding to any amino acid other than alanine at position 2178 of SEQ ID NO: 2.
557. A plant or plant cell expressing an ACCase protein comprising a substitution corresponding to any amino acid other than alanine at position 2179 of SEQ ID NO: 2.
558. A plant or plant cell expressing an ACCase protein comprising a substitution corresponding to any amino acid other than valine at position 2182 of SEQ ID NO: 2.
559. A plant or plant cell expressing an ACCase protein comprising a substitution corresponding to any amino acid other than phenylalanine at position 2196 of SEQ ID NO: 2.
560. A plant or plant cell expressing an ACCase protein comprising a substitution corresponding to any amino acid other than lysine at position 2220 of SEQ ID NO: 2.
561. A plant or plant cell expressing an ACCase protein comprising a substitution corresponding to any amino acid other than histidine at position 2223 of SEQ ID NO: 2.
562. A plant or plant cell expressing an ACCase protein comprising a substitution corresponding to any amino acid other than alanine at position 2226 of SEQ ID NO: 2.
563. A plant or plant cell expressing an ACCase protein comprising a substitution corresponding to any amino acid other than lysine at position 2232 of SEQ ID NO: 2.
564. A plant or plant cell expressing an ACCase protein comprising a substitution corresponding to any amino acid other than serine at position 2244 of SEQ ID NO: 2.
565. A plant or plant cell expressing an ACCase protein comprising a substitution corresponding to any amino acid other than phenylalanine at position 2253 of SEQ ID NO: 2.
566. A plant or plant cell expressing an ACCase protein comprising a substitution corresponding to any amino acid other than valine at position 2254 of SEQ ID NO: 2.
567. A plant or plant cell expressing an ACCase protein comprising a substitution corresponding to any amino acid other than arginine at position 2257 of SEQ ID NO: 2.
568. A plant or plant cell expressing an ACCase protein comprising a substitution corresponding to any amino acid other than asparagine at position 2262 of SEQ ID NO: 2.
569. A plant or plant cell expressing an ACCase protein comprising a substitution corresponding to any amino acid other than serine at position 2276 of SEQ ID NO: 2.
570. A plant or plant cell expressing an ACCase protein comprising a substitution corresponding to any amino acid other than serine at position 2280 of SEQ ID NO: 2.
571. A plant or plant cell expressing an ACCase protein comprising a substitution corresponding to any amino acid other than serine at position 2297 of SEQ ID NO: 2.
572. A plant or plant cell expressing an ACCase protein comprising a substitution corresponding to any amino acid other than leucine at position 2299 of SEQ ID NO: 2.
573. A plant or plant cell expressing an ACCase protein comprising a substitution corresponding to any amino acid other than lysine at position 2303 of SEQ ID NO: 2.
574. A plant or plant cell expressing an ACCase protein comprising a substitution corresponding to any amino acid other than lysine at position 2317 of SEQ ID NO: 2.
575. A plant or plant cell expressing an ACCase protein comprising one or more substitution combinations corresponding to one or more combinations selected from the group consisting of V2075L/N2097S; V2075I/N2097S; D2078G/N2097S; V2098A/N2097S; and V2098G/N2097S of SEQ ID NO: 2.
576. A plant or plant cell expressing an ACCase protein comprising one or more substitution combinations corresponding to V2075L/N2097S of SEQ ID NO: 2.
577. A plant or plant cell expressing an ACCase protein comprising one or more substitution combinations corresponding to V2075I/N2097S of SEQ ID NO: 2.
578. A plant or plant cell expressing an ACCase protein comprising one or more substitution combinations corresponding to D2078G/N2097S of SEQ ID NO: 2.
579. A plant or plant cell expressing an ACCase protein comprising one or more substitution combinations corresponding to V2098A/N2097S of SEQ ID NO: 2.
580. A plant or plant cell expressing an ACCase protein comprising one or more substitution combinations corresponding to V2098G/N2097S of SEQ ID NO: 2.
581. A plant or plant cell expressing an ACCase protein comprising an amino acid substitution at a position corresponding to N2097 of SEQ ID NO: 2, wherein the substitution is selected from the group consisting N2097R, N2097K, N2097E, N2097H, N2097D, N2097A, N2097G, N2097C, N2097V, N2097P, N2097L, N2097I, N2097M, N2097F, N2097W, N2097Q, N2097S, N2097T, and N2097Y.
582. A plant or plant cell expressing an ACCase protein comprising a substitution corresponding to N2097 of SEQ ID NO: 2 (such as a substitution as provided herein) in combination with an additional ACCase mutation or mutations as provided herein.
583. A plant or plant cell expressing an ACCase protein comprising a substitution corresponding to N2097 of SEQ ID NO: 2 (such as a substitution as provided herein) in combination with an additional ACCase mutation or mutations as provided in Table 1.
584. A plant or plant cell expressing an ACCase protein comprising a substitution corresponding to N2097 of SEQ ID NO: 2 (such as a substitution as provided herein) in combination with an additional ACCase mutation or mutations as provided in Table 2.
585. A plant or plant cell expressing an ACCase protein comprising a substitution corresponding to N2097 of SEQ ID NO: 2 (such as a substitution as provided herein) in combination with an additional ACCase mutation as provided in Table 3.
586. A plant or plant cell expressing an ACCase protein comprising a substitution corresponding to N2097 of SEQ ID NO: 2 (such as a substitution as provided herein) in combination with an additional ACCase mutation at an amino acid position corresponding to 2075 of SEQ ID NO: 2.
587. A plant or plant cell expressing an ACCase protein comprising a substitution corresponding to N2097 of SEQ ID NO: 2 (such as a substitution as provided herein) in combination with an additional ACCase mutation comprising a substitution corresponding to V20751 of SEQ ID NO: 2.
588. A plant or plant cell expressing an ACCase protein comprising a substitution corresponding to N2097 of SEQ ID NO: 2 (such as a substitution as provided herein) in combination with an additional ACCase mutation comprising a substitution corresponding to V2075L of SEQ ID NO: 2.
589. A plant or plant cell expressing an ACCase protein comprising a substitution corresponding to N2097 of SEQ ID NO: 2 (such as a substitution as provided herein) in combination with an additional ACCase mutation at an amino acid position corresponding to 2078 of SEQ ID NO: 2.
590. A plant or plant cell expressing an ACCase protein comprising a substitution corresponding to N2097 of SEQ ID NO: 2 (such as a substitution as provided herein) in combination with an additional ACCase mutation comprising a substitution corresponding to D2078G of SEQ ID NO: 2.
591. A plant or plant cell expressing an ACCase protein comprising a substitution corresponding to N2097 of SEQ ID NO: 2 (such as a substitution as provided herein) in combination with an additional ACCase mutation at an amino acid position corresponding to 2098 of SEQ ID NO: 2.
592. A plant or plant cell expressing an ACCase protein comprising a substitution corresponding to N2097 of SEQ ID NO: 2 (such as a substitution as provided herein) in combination with an additional ACCase mutation comprising a substitution corresponding to V2098A of SEQ ID NO: 2.
593. A plant or plant cell expressing an ACCase protein comprising a substitution corresponding to N2097 of SEQ ID NO: 2 (such as a substitution as provided herein) in combination with an additional ACCase mutation comprising a substitution corresponding to V2098G of SEQ ID NO: 2.
594. A plant or plant cell expressing an ACCase protein comprising one or more substitution combinations corresponding to V2098G/A2226S of SEQ ID NO: 2.
595. A plant or plant cell expressing an ACCase protein comprising one or more substitution combinations corresponding to one or more combinations selected from the group consisting of L1543V/D1681E; L1543F/D1681E; L1543V/D1681G; L1543F/D1681G; A1785G/I1781L; A1785G/I1781T; A1785G/I1781V; A1785G/C2088M; A1785G/C2088N; A1785G/C2088R; L1818H/S1784N; L1818H/S1784G; L1818I/S1784N; L1818I/S1784G; V1529M/L1818H; V1529M/L1818I; K1840R/L1818H; K1840R/L1818I; K1840M/L1818H; K1840M/L1818I; N2097S/D2078G; N2097D/D2078G; N2097S/V2075L; N2097D/V2075L; N2097S/V2075I; N2097D/V2075I; N2097S/V2098A; N2097D/V2098A; N2097S/V2098G; N2097D/V2098G; R1672I/D2078G; M1974T/D2078G; D2078G/P2083T; D2078G/C2088M; I1781L/V2075L; D2078G/C2088N; D2078G/C2088R; I1781L/V2075I; I1781T/V2075I; I1781T/V2075L; I1781V/V2075I; I1781V/V2075L; of SEQ ID NO: 2.
596. A plant or plant cell expressing an ACCase protein comprising one or more substitution combinations corresponding to L1543V/D1681E of SEQ ID NO: 2.
597. A plant or plant cell expressing an ACCase protein comprising one or more substitution combinations corresponding to L1543F/D1681E of SEQ ID NO: 2.
598. A plant or plant cell expressing an ACCase protein comprising one or more substitution combinations corresponding to L1543V/D1681G of SEQ ID NO: 2.
599. A plant or plant cell expressing an ACCase protein comprising one or more substitution combinations corresponding to L1543F/D1681G of SEQ ID NO: 2.
600. A plant or plant cell expressing an ACCase protein comprising one or more substitution combinations corresponding to one or more combinations selected from the group consisting of S1784G/A1785G; S1784N/A1785G; A1785G/I1781L; A1785G/I1781T; A1785G/I1781V; A1785G/C2088M; A1785G/C2088N; A1785G/C2088R; L1818H/S1784N; L1818H/S1784G; L1818I/S1784N; and L1818I/S1784G of SEQ ID NO: 2.
601. A plant or plant cell expressing an ACCase protein comprising one or more substitution combinations corresponding to S1784G/A1785G of SEQ ID NO: 2.
602. A plant or plant cell expressing an ACCase protein comprising one or more substitution combinations corresponding to S1784N/A1785G of SEQ ID NO: 2.
603. A plant or plant cell expressing an ACCase protein comprising one or more substitution combinations corresponding to A1785G/I1781L of SEQ ID NO: 2.
604. A plant or plant cell expressing an ACCase protein comprising one or more substitution combinations corresponding to A1785G/I1781T of SEQ ID NO: 2.
605. A plant or plant cell expressing an ACCase protein comprising one or more substitution combinations corresponding to A1785G/I1781V of SEQ ID NO: 2.
606. A plant or plant cell expressing an ACCase protein comprising one or more substitution combinations corresponding to A1785G/C2088M of SEQ ID NO: 2.
607. A plant or plant cell expressing an ACCase protein comprising one or more substitution combinations corresponding to A1785G/C2088N of SEQ ID NO: 2.
608. A plant or plant cell expressing an ACCase protein comprising one or more substitution combinations corresponding to A1785G/C2088R of SEQ ID NO: 2.
609. A plant or plant cell expressing an ACCase protein comprising one or more substitution combinations corresponding to L1818H/S1784N of SEQ ID NO: 2.
610. A plant or plant cell expressing an ACCase protein comprising one or more substitution combinations corresponding to L1818H/S1784G of SEQ ID NO: 2.
611. A plant or plant cell expressing an ACCase protein comprising one or more substitution combinations corresponding to L1818I/S1784N of SEQ ID NO: 2.
612. A plant or plant cell expressing an ACCase protein comprising one or more substitution combinations corresponding to L1818I/S1784G of SEQ ID NO: 2.
613. A plant or plant cell expressing an ACCase protein comprising one or more substitution combinations corresponding to V1529M/L1818H of SEQ ID NO: 2.
614. A plant or plant cell expressing an ACCase protein comprising one or more substitution combinations corresponding to V1529M/L1818I of SEQ ID NO: 2.
615. A plant or plant cell expressing an ACCase protein comprising one or more substitution combinations corresponding to K1840R/L1818H of SEQ ID NO: 2.
616. A plant or plant cell expressing an ACCase protein comprising one or more substitution combinations corresponding to K1840R/L18181 of SEQ ID NO: 2.
617. A plant or plant cell expressing an ACCase protein comprising one or more substitution combinations corresponding to K1840M/L1818H of SEQ ID NO: 2.
618. A plant or plant cell expressing an ACCase protein comprising one or more substitution combinations corresponding to K1840M/L1818I of SEQ ID NO: 2.
619. A plant or plant cell expressing an ACCase protein comprising one or more substitution combinations corresponding to N2097S/D2078G of SEQ ID NO: 2.
620. A plant or plant cell expressing an ACCase protein comprising one or more substitution combinations corresponding to N2097D/D2078G of SEQ ID NO: 2.
621. A plant or plant cell expressing an ACCase protein comprising one or more substitution combinations corresponding to N2097S/V2075L of SEQ ID NO: 2.
622. A plant or plant cell expressing an ACCase protein comprising one or more substitution combinations corresponding to N2097D/V2075L of SEQ ID NO: 2.
623. A plant or plant cell expressing an ACCase protein comprising one or more substitution combinations corresponding to N2097S/V20751 of SEQ ID NO: 2.
624. A plant or plant cell expressing an ACCase protein comprising one or more substitution combinations corresponding to N2097D/V2075I of SEQ ID NO: 2.
625. A plant or plant cell expressing an ACCase protein comprising one or more substitution combinations corresponding to N2097S/V2098A of SEQ ID NO: 2.
626. A plant or plant cell expressing an ACCase protein comprising one or more substitution combinations corresponding to N2097D/V2098A of SEQ ID NO: 2.
627. A plant or plant cell expressing an ACCase protein comprising one or more substitution combinations corresponding to N2097S/V2098G of SEQ ID NO: 2.
628. A plant or plant cell expressing an ACCase protein comprising one or more substitution combinations corresponding to N2097D/V2098G of SEQ ID NO: 2.
629. A plant or plant cell expressing an ACCase protein comprising one or more substitution combinations corresponding to R1672I/D2078G of SEQ ID NO: 2.
630. A plant or plant cell expressing an ACCase protein comprising one or more substitution combinations corresponding to M1974T/D2078G of SEQ ID NO: 2.
631. A plant or plant cell expressing an ACCase protein comprising one or more substitution combinations corresponding to D2078G/P2083T of SEQ ID NO: 2.
632. A plant or plant cell expressing an ACCase protein comprising one or more substitution combinations corresponding to D2078G/C2088M of SEQ ID NO: 2.
633. A plant or plant cell expressing an ACCase protein comprising one or more substitution combinations corresponding to D2078G/C2088N of SEQ ID NO: 2.
634. A plant or plant cell expressing an ACCase protein comprising one or more substitution combinations corresponding to D2078G/C2088R of SEQ ID NO: 2.
635. A plant or plant cell expressing an ACCase protein comprising one or more substitution combinations corresponding to I1781L/V2075I of SEQ ID NO: 2.
636. A plant or plant cell expressing an ACCase protein comprising one or more substitution combinations corresponding to I1781L/V2075L of SEQ ID NO: 2.
637. A plant or plant cell expressing an ACCase protein comprising one or more substitution combinations corresponding to I1781T/V2075I of SEQ ID NO: 2.
638. A plant or plant cell expressing an ACCase protein comprising one or more substitution combinations corresponding to I1781T/V2075L of SEQ ID NO: 2.
639. A plant or plant cell expressing an ACCase protein comprising one or more substitution combinations corresponding to I1781V/V2075I of SEQ ID NO: 2.
640. A plant or plant cell expressing an ACCase protein comprising one or more substitution combinations corresponding to I1781V/V2075L of SEQ ID NO: 2.
641. A plant or plant cell expressing an ACCase protein comprising one or more mutations resulting a substitution combination corresponding to one or more combinations selected from the group consisting of V1529M/L1818H; L1555P/S1605Y/L1985P/D2078G; C1575S/G1605-6A*/V2075L; S1602F/D1725E/I1781T/V2075L; S1603N/L1649M/V1715A/D2078G; A1957T/V2075L; I1967V/V2075L/S2119C; M1975T/D2078G; R1672I/M1975T/D2078G; R1672I/D2078G; I1766V/D2078G/D2127N; S1605Y/V2098A; V1966L/D2078G/V2182A/A2244T/K2317T; F1683L/A1752T/K1930N/V2075L/E2169D; K1772E/S1784N/V1924D; E1946V/D2078G/A2179T/F2253I; M1974T/D2078G/M2257I; M1974T/D2078G; D2078G/M2257I; V1992D/V2075L/F2196Y/L2299I; V2049I/V2075L/A2178I; A2059V/D2078G/S2280P/S2297C; A2059V/D2078G; M2065K/D2078G/R2085S/Q2155L/Q2220L; A2067S/V2075L/L2159P; V2075L/N2097S; D2078G; D2078G/P2083T; D2078G/N2097S/H2223R/S2276F; D2078G/E2112V; D2078G/12149K; D2078G/E2150K/12267V; D2078G/K2154R/K2232N; D2078G/N2262K; D2078G/S2276P/K2302Q; V2098A/V21341/A2151T/V2254I; V2098G; and V2098G/A2226S of SEQ ID NO: 2.
642. A plant or plant cell expressing an ACCase protein comprising one or more substitution combinations corresponding to V1529M/L1818H of SEQ ID NO: 2.
643. A plant or plant cell expressing an ACCase protein comprising one or more substitution combinations corresponding to L1555P/S1605Y/L1985P/D2078G of SEQ ID NO: 2.
644. A plant or plant cell expressing an ACCase protein comprising one or more substitution combinations corresponding to C1575S/G1605-6A*/V2075L of SEQ ID NO: 2.
645. A plant or plant cell expressing an ACCase protein comprising one or more substitution combinations corresponding to D1725E/I1781T/V2075L of SEQ ID NO: 2.
646. A plant or plant cell expressing an ACCase protein comprising one or more substitution combinations corresponding to S1603N/L1649M/V1715A/D2078G of SEQ ID NO: 2.
647. A plant or plant cell expressing an ACCase protein comprising one or more substitution combinations corresponding to S1605Y/V2098A of SEQ ID NO: 2.
648. A plant or plant cell expressing an ACCase protein comprising one or more substitution combinations corresponding to A1957T/V2075L of SEQ ID NO: 2.
649. A plant or plant cell expressing an ACCase protein comprising one or more substitution combinations to corresponding V1966L/D2078G/V2182A/A2244T/K2317T of SEQ ID NO: 2.
650. A plant or plant cell expressing an ACCase protein comprising one or more substitution combinations corresponding to I1967V/V2075L/S2119C of SEQ ID NO: 2.
651. A plant or plant cell expressing an ACCase protein comprising one or more substitution combinations corresponding to R1672I/M1975T/D2078G of SEQ ID NO: 2.
652. A plant or plant cell expressing an ACCase protein comprising one or more substitution combinations corresponding to R1672I/D2078G of SEQ ID NO: 2.
653. A plant or plant cell expressing an ACCase protein comprising one or more substitution combinations corresponding to M1975T/D2078G of SEQ ID NO: 2.
654. A plant or plant cell expressing an ACCase protein comprising one or more substitution combinations corresponding to F1683L/A1752T/K1930N/V2075L/E2169D of SEQ ID NO: 2.
655. A plant or plant cell expressing an ACCase protein comprising one or more substitution combinations corresponding to I1766V/D2078G/D2127N of SEQ ID NO: 2.
656. A plant or plant cell expressing an ACCase protein comprising one or more substitution combinations corresponding to K1772E/S1784N/V1924D of SEQ ID NO: 2.
657. A plant or plant cell expressing an ACCase protein comprising one or more substitution combinations corresponding to E1946V/D2078G/A2179T/F2253I of SEQ ID NO: 2.
658. A plant or plant cell expressing an ACCase protein comprising one or more substitution combinations corresponding to M1974T/D2078G/M22571 of SEQ ID NO: 2.
659. A plant or plant cell expressing an ACCase protein comprising one or more substitution combinations corresponding to M1974T/D2078G; D2078G/M22571 of SEQ ID NO: 2.
660. A plant or plant cell expressing an ACCase protein comprising one or more substitution combinations corresponding to V1992D/V2075L/F2196Y/L2299I of SEQ ID NO: 2.
661. A plant or plant cell expressing an ACCase protein comprising one or more substitution combinations corresponding to V2049I/V2075L/A21781 of SEQ ID NO: 2.
662. A plant or plant cell expressing an ACCase protein comprising one or more substitution combinations corresponding to A2059V/D2078G/S2280P/S2297C of SEQ ID NO: 2.
663. A plant or plant cell expressing an ACCase protein comprising one or more substitution combinations corresponding to A2059V/D2078G of SEQ ID NO: 2.
664. A plant or plant cell expressing an ACCase protein comprising one or more substitution combinations corresponding to M2065K/D2078G/R2085S/Q2155L/Q2220L of SEQ ID NO: 2.
665. A plant or plant cell expressing an ACCase protein comprising one or more substitution combinations corresponding to A2067S/V2075L/L2159P of SEQ ID NO: 2.
666. A plant or plant cell expressing an ACCase protein comprising one or more substitution combinations corresponding to V2075L/N2097S of SEQ ID NO: 2.
667. A plant or plant cell expressing an ACCase protein comprising one or more substitution combinations corresponding to D2078G of SEQ ID NO: 2.
668. A plant or plant cell expressing an ACCase protein comprising one or more substitution combinations corresponding to D2078G/P2083T of SEQ ID NO: 2.
669. A plant or plant cell expressing an ACCase protein comprising one or more substitution combinations corresponding to D2078G/N2097S/H2223R/S2276F of SEQ ID NO: 2.
670. A plant or plant cell expressing an ACCase protein comprising one or more substitution combinations corresponding to D2078G/E2112V of SEQ ID NO: 2.
671. A plant or plant cell expressing an ACCase protein comprising one or more substitution combinations corresponding to D2078G/12149K of SEQ ID NO: 2.
672. A plant or plant cell expressing an ACCase protein comprising one or more substitution combinations corresponding to D2078G/E2150K/12267V of SEQ ID NO: 2.
673. A plant or plant cell expressing an ACCase protein comprising one or more substitution combinations corresponding to D2078G/K2154R/K2232N of SEQ ID NO: 2.
674. A plant or plant cell expressing an ACCase protein comprising one or more substitution combinations corresponding to D2078G/N2262K of SEQ ID NO: 2.
675. A plant or plant cell expressing an ACCase protein comprising one or more substitution combinations corresponding to D2078G/S2276P/K2302Q of SEQ ID NO: 2.
676. A plant or plant cell expressing an ACCase protein comprising one or more substitution combinations corresponding to V2098A/V21341/A2151T/V2254I of SEQ ID NO: 2.
677. A plant or plant cell expressing an ACCase protein comprising one or more substitution combinations corresponding to V2098G of SEQ ID NO: 2.
678. A plant or plant cell expressing an ACCase protein comprising one or more substitution combinations corresponding to V2098G/A2226S of SEQ ID NO: 2.
679. The plant or plant cell of any of the preceding embodiments wherein such plant or plant cell resistant to one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, or an agronomically acceptable salts or esters thereof.
680. The plant or plant cell of any of the preceding claims wherein such plant or plant cell is resistant to clethodim.
681. The plant or plant cell of any of the preceding claims wherein such plant or plant cell is fertile.
682. The plant or plant cell of any of the preceding embodiments wherein the plant species is selected from the group consisting of canola, sunflower, corn, tobacco, sugar beet, cotton, maize, wheat (including but not limited to Triticum spp., Triticum aestivum, Triticum durum Triticum timopheevii, Triticum monococcum, Triticum spelta, Triticum zhukovskyi and Triticum urartu and hybrids thereof), barley (including but not limited to Hordeum vulgare L., Hordeum comosum, Hordeum depressum, Hordeum intercedens, Hordeum jubatum, Hordeum marinum, Hordeum marinum, Hordeum parodii, Hordeum pusillum, Hordeum secalinum, and Hordeum spontaneum), rice (including but not limited to Oryza sativa subsp. indica, Oryza sativa subsp. japonica, Oryza sativa subsp. javanica, Oryza sativa subsp. glutinosa (glutinous rice), Oryza sativa Aromatica group (e.g., basmati), and Oryza sativa (floating rice group)), alfalfa, sorghum (e.g., Sorghum bicolor), foxtail millet (e.g., Setaria italica), switchgrass (e.g., Panicum virgatum), Kentucky blue-grass (e.g., Poa pratensis), tomato, mango, peach, apple, pear, strawberry, banana, melon, cassava, potato, carrot, lettuce, onion, soy bean, soya spp, sugar cane, pea, chickpea, field pea, fava bean, lentils, turnip, rutabaga, brussels sprouts, lupin, cauliflower, kale, field beans, poplar, pine, eucalyptus, grape, citrus, triticale, alfalfa, rye (including but not limited to Secale sylvestre, Secale strictum, Secale cereale, Secale vavilovii, Secale africanum, Secale ciliatoglume, Secale ancestrale, and Secale montanum), perennial ryegrass (e.g., Lolium perenne), oats (Avena sativa), turf (including but not limited to Turf grass include Zoysia japonica, Agrostris palustris, Poa pratensis, Poa annua, Digitaria sanguinalis, Cyperus rotundus, Kyllinga brevifolia, Cyperus amuricus, Erigeron canadensis, Hydrocotyle sibthorpioides, Kummerowia striata, Euphorbia humifusa, and Viola arvensis) and forage grasses, flax, oilseed rape, cotton, mustard, cucumber, morning glory, balsam, pepper, eggplant, marigold, lotus, cabbage, daisy, carnation, tulip, iris, and lily.
683. The plant or plant cell of any of the preceding embodiments, wherein the plant or plant cell belongs to the genus Oryza.
684. The plant or plant cell of any of the preceding embodiments, wherein said plant or plant cell is transgenic.
685. The plant or plant cell of any of the preceding embodiments, wherein said plant or plant cell is non-transgenic.

### EXAMPLES

The following examples are provided to further illustrate aspects of the present disclosure. These examples are non-limiting and should not be construed as limiting any aspect of the present disclosure.

Methods to measure herbicide tolerance of mutations listed here within are well known in the art. Joachimiak M. et al Proc Natl Acad Sci U.S.A. 1997 Sep. 2; 94 (18): 9990-5 and WO/2013/074524 and WO/2013/074524 each describe methods to complement a yeast (Saccharomyces cerevisiae) strain lacking the native acc1 gene with either a wt or mutated form of a chimeric plastidal ACCase gene from a plant such as rice (Oryza sativa). Methods to introduce one or more mutations listed herein into the chimeric ACCase gene and subsequently complement the acc1 lacking yeast strain are well known by an individual skilled in the art. In the art assays are known for measuring tolerance of a mutated plant ACCase gene versus a herbicide that is a member of the aryloxyphenoxypropanoate, cyclohexanedione, and/or phenylpyrazoline families of herbicides such as clethodim, sethoxydim, fluazifop, haloxyfop, quizalofop, and pinoxaden (Joachimiak M. et al Proc Natl Acad Sci U.S.A. 1997 Sep. 2; 94 (18): 9990-5 and WO/2013/074524).

Methods to measure herbicide tolerance of whole plants, such as rice (Oryza sativa), containing one or more mutations listed herein are known in the art. WO/2011/028833, WO/2013/016210, WO/2012/106321 and Neve and Powles, Heredity 2005 95, 485-492 detail procedures to assess tolerance of whole plants containing one or more ACCase mutations derived from random mutagenesis, somaclonal variation and transgenic procedures and such procedures are general applicable to plants produced by essentially any other method known in the art. The assays can be used by an individual skilled in the art to determine the tolerance of a plant with any one or more mutations listed herein versus an herbicide that is a member of the aryloxyphenoxypropanoate, cyclohexanedione, and/or phenylpyrazoline families of herbicides such as clethodim, sethoxydim, fluazifop, haloxyfop, quizalofop, and pinoxaden.

### Example 1

Three amino acid changes between the plastidal and cytoplasmic enzymes correlate with mutations known to produce tolerance to ACCase inhibitors. Specific positions are listed in Table 4 below with amino acid numbering relative to the reference blackgrass (Alopecurus myosuroides) Alopecurus myosuroides ((Am) FIG. 1B). This suggests that some combination of the I1781L, A1785G and/or C2088M mutations produces significant levels of herbicide tolerance.

TABLE 4

| | | Known Herbicide | | |
|---|---|---|---|---|
| Reference | wt | Tolerant | wt OsACCase | |
| Am Position | Residue | Mutations | Plastidal | Cytosolic |
| 1781 | I | L, T, V, A | I | L |
| 1785 | A | G | A | G |
| 2088 | C | R, T | C | M |

Measuring tolerance of each individual and all possible combinatorial mutations to the herbicide clethodim demonstrates that the I1781L mutation produces tolerance. The C2088M mutation also produces measurable tolerance whereas the A1785G mutation yields no detectable clethodim tolerance. Strikingly, combining the A1785G mutation with either the I1781L or the C2088M mutation produces an increase in herbicide tolerance for each double mutant combination that is measurably higher than is expected if tolerance of each single mutation was additive. This Example showing that A1785G is an enhancer of clethodim tolerance is an unexpected finding because A1785G alone does not produce detectable clethodim tolerance.

### Example 2

A random mutagenesis method was used to identify new mutations providing herbicide tolerance. A library of random mutations in the rice (Oryza sativa) plastidal ACCase gene was generated and screened for clethodim tolerant clones using a yeast complementation assay. Mutations L1543F, L1543V, D1681E, D1681G, S1784G, S1784N, L1818I, L1818H, D2078G, V2075L, and V2098A produce measurable clethodim tolerance. Combining an L1543 substitution with a D1691 change, the double mutants L1543F-D1681E, L1543F-D1681G, L1543V-D1681E, and L1543V-D1681G increased clethodim tolerance compared to any one mutation alone. The double mutant S1784N plus L1818H also improves tolerance in comparison to either single mutation, and many of the tolerant clones have the D2078G change in combination with one or more additional mutations. Specifically, the triple mutant R1672I/M1975T/D2078G and M1974T/D2078G/M2257I clones show increased clethodim tolerance compared to the single D2078G mutation. Measuring clethodim tolerance of a series of single and double mutant combinations derived from each of the above triple mutants in comparison to the single D2078G mutation and the original triple mutant showed: (a) no single mutant other than D2078G tested in this Example demonstrates measurable clethodim tolerance in this assay; (b) both R1672I and M1974T increase tolerance of the single D2078G mutation; (c) the double R1672I-D2078G and M1974T-D2078G mutants show tolerance that is similar to the corresponding original triple mutants; and (d) neither M1975T nor M2257I provide detectable clethodim tolerance increase to the single D2078G mutation in this assay. Together, this Example indicates that both the R1672I and M1974T mutations enhance clethodim tolerance of the D2078G change in the rice ACCase enzyme, an unexpected finding because neither R1672I nor M1974T alone provide measurable tolerance.

### Example 3

A N2097D substitution is reportedly associated with tolerance to the fluazifop herbicide (Cha et al., Pesticide Biochemistry and Physiology 111 (2014) 7-13). Clethodim tolerance testing with a yeast complementation assay shows that neither the N2097S nor the N2097D single mutations provide detectable clethodim tolerance. Combining N2097S with either D2078G, V2075L or V2098A further increases clethodim tolerance; that is, the double mutants D2078G/N2097S, V2075L/N2097D and V2098A/N2097D are either less, or no more, tolerant than the single D2078G, V2075L and V2098A mutations. It is believed that N2097S is an enhancer of clethodim herbicide tolerance, even though N2097S alone does not produce detectable clethodim tolerance. Strikingly, combining N2097D with either D2078G, V2075L or V2098A produces opposing results. The N2097S mutation, but not N2097D, also shows similar enhancer activity using the ACCase inhibitor Quizalofop to measure herbicide tolerance. This Example indicates that the N2097S mutation is an enhancer of herbicide tolerance whereas N2097D produces opposing phenotypic output.

### Example 4

A method to introduce targeted mutations into a native rice Oryza sativa ACCase gene was used utilizing PEG delivery of CRISPR-Cas9 and GRONs into protoplasts. The CRISPR-Cas9 consists of two components: Cas9 protein, such as a plant codon-optimized Streptococcus pyogenes Cas9 (SpCas9) and sgRNA, both of which can be expressed from plasmid(s). Cas9 and sgRNA can also be delivered by mRNA/RNA or protein/RNA respectively. The sgRNA is a fusion of CRISPR RNA (crRNA) and trans-activating crRNA (tracrRNA). The crRNA region contains a spacer used to guide the Cas9 nuclease to the target location in the ACCase gene where Cas9 makes either a double-stranded break or a nick at a targeted site and the GRONs are used as a template to edit the ACCase gene in a site-directed manner. Examples of target mutations include, but are not limited to, those listed in the above Table 1, Table 2, and Table 3.

Examples of target spacer sequences for sgRNA targeting the rice ACCase gene are shown, but not limited to, those listed in the below Table 5.

TABLE 5
Exemplary target spacer
sequences for rice ACCase sgRNA

| Sequence No. | 5′ to 3′ sequence |
|---|---|
| 1 (SEQ ID NO: 29) | aauggaagauaaagaauca |
| 2 (SEQ ID NO: 30) | ggauggaagauaaagaauca |
| 3 (SEQ ID NO: 31) | cagccgccggaguggcggga |
| 4 (SEQ ID NO: 32) | gagccgccggaguggcggga |
| 5 (SEQ ID NO: 33) | aaugauauuacguucagagc |
| 6 (SEQ ID NO: 34) | gaugauauuacguucagagc |
| 7 (SEQ ID NO: 35) | acguucagagcuggaucauu |
| 8 (SEQ ID NO: 36) | gcguucagagcuggaucauu |
| 9 (SEQ ID NO: 37) | ucaaaaaaugcaucuucccu |
| 10 (SEQ ID NO: 38) | gcaaaaaaugcaucuucccu |
| 11 (SEQ ID NO: 39) | gagcuggaucauuuggccca |
| 12 (SEQ ID NO: 40) | cuugguguggagaauauaca |
| 13 (SEQ ID NO: 41) | guugguguggagaauauaca |
| 14 (SEQ ID NO: 42) | auaugcccuagaauaagcac |
| 15 (SEQ ID NO: 43) | guaugcccuagaauaagcac |
| 16 (SEQ ID NO: 44) | ggagcuuaucuugcucgacu |
| 17 (SEQ ID NO: 45) | ggucaagacgcuguaugcac |
| 18 (SEQ ID NO: 46) | aucuugcucgacuuggcauc |
| 19 (SEQ ID NO: 47) | gucuugcucgacuuggcauc |
| 20 (SEQ ID NO: 48) | uccagugggugugauagcug |
| 21 (SEQ ID NO: 49) | gccagugggugugauagcug |
| 22 (SEQ ID NO: 50) | agcagggauaguuugcauca |
| 23 (SEQ ID NO: 51) | ggcagggauaguuugcauca |
| 24 (SEQ ID NO: 52) | guugguguggugaauauaca |
| 25 (SEQ ID NO: 53) | cuugguguggagaauauaca |
| 26 (SEQ ID NO: 54) | gugggcaaggaagauggacu |
| 27 (SEQ ID NO: 55) | auaugcccaagaauaagcac |
| 28 (SEQ ID NO: 56) | guauggccuagaauaagcac |
| 29 (SEQ ID NO: 57) | gacugcaaaaggcaauguuc |
| 30 (SEQ ID NO: 58) | gaugcugagaggacugcaaa |
| 31 (SEQ ID NO: 59) | uaugcugagaggacugcaaa |
| 32 (SEQ ID NO: 60) | ggcaauguucuggaaccgca |
| 33 (SEQ ID NO: 61) | uugaucucaauuaacccuug |
| 34 (SEQ ID NO: 62) | gugaucucaauuaacccuug |
| 35 (SEQ ID NO: 63) | gcgaggaggggcuuggguug |
| 36 (SEQ ID NO: 64) | agaggaggggcuuggguug |
| 37 (SEQ ID NO: 65) | ggcugcagagcuacgaggag |
| 38 (SEQ ID NO: 66) | cguaucgugaguuacgccag |
| 39 (SEQ ID NO: 67) | uccucguagcucugcagcca |
| 40 (SEQ ID NO: 68) | gccucguagcucugcagcca |
| 41 (SEQ ID NO: 69) | caaggguuaauugagaucaa |
| 42 (SEQ ID NO: 70) | gaaggguuaauugagaucaa |

Rice protoplasts were isolated from cell suspension cultures derived from mature seed-derived embryogenic calli. CRISPR-Cas encoding plasmids, mRNA and/or protein with GRONs were introduced into protoplasts by PEG mediated delivery, by electroporation or any other means of delivering cargo into plant cells. An exemplary range for CRISPR-Cas plasmids at a final concentration includes but is not limited to 0.01 to 0.2 μg/μl. An exemplary range for CRISPR-Cas mRNA at a final concentration includes but is not limited to 0.1 μg/μl to 2.0 μg/μl. An exemplary range for CRISPR-Cas protein at a final concentration include but is not limited to 0.05 μg/μl to 2.0 μg/μl. An exemplary range for GRON at a final concentration includes but is not limited to 0.01 to 4 μM. Sequence information for exemplary GRONs is shown, but not limited to those listed in the below Table 6.

TABLE 6
Exemplary rice ACCase GRON Sequences

| Sequence No. | 5′ to 3′ sequence (V = CY3; H = 3′DMT dC CPG) |
|---|---|
| 1 (SEQ ID NO: 71) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATATACATGGAAGTGCTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 2 (SEQ ID NO: 72) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATCTTCATGGAAGTGCTGCTATTGCCAGTGCT |
| | TATTCTAGGGCATATAAGGAGACATTTACACTTACATTTGTG |
| | ACTGGAAGAACTGTTGGAATAGGAGCTTATCTTGCTCH |
| 3 (SEQ ID NO: 73) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATCTCCATGGAAGTGCTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 4 (SEQ ID NO: 74) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATCTACATGGAAGTGCTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 5 (SEQ ID NO: 75) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATCTGCATGGAAGTGCTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 6 (SEQ ID NO: 76) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATACUCATGGAAGTGCTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 7 (SEQ ID NO: 77) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATACCCATGGAAGTGCTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 8 (SEQ ID NO: 78) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATACACATGGAAGTGCTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 9 (SEQ ID NO: 79) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATACGCATGGAAGTGCTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 10 (SEQ ID NO: 80) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATCTTCATGGAAGTGGAGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 11 (SEQ ID NO: 81) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATCTCCATGGAAGTGGGGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 12 (SEQ ID NO: 82) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATCTACATGGAAGTGGTGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 13 (SEQ ID NO: 83) | VGTCATAGCACATAAGATGCAGCTAGACAGTGGTGAAATTA |
| | GGTGGGTTATTGATTCTGTTGTGGGCAAGGAAGATGGACTT |
| | GGTGTGGAGAATCTGCATGGAAGTAACGCTATTGCCAGTGC |
| | TTATTCTAGGGCATATAAGGAGACATTTACACTTACATTTGT |
| | GACTGGAAGAACTGTTGGAATAGGAGCTTATCTTGCTCH |
| 14 (SEQ ID NO: 84) | VGGGTTATTGATTCTGTTGTGGGCAAGGAAGATGGACTTGG |
| | TGTGGAGAATATACATGGAAGTGCTGCTATTGCCAGTGCTT |
| | ATTCTAGGGCATATAAGGAGACATTTACACTTAH |
| 15 (SEQ ID NO: 85) | VGGGTTATTGATTCTGTTGTGGGCAAGGAAGATGGACTTGG |
| | TGTGGAGAATCTTCATGGAAGTGCTGCTATTGCCAGTGCTTA |
| | TTCTAGGGCATATAAGGAGACATTTACACTTAH |
| 16 (SEQ ID NO: 86) | VGGGTTATTGATTCTGTTGTGGGCAAGGAAGATGGACTTGG |
| | TGTGGAGAATCTCCATGGAAGTGCTGCTATTGCCAGTGCTTA |
| | TTCTAGGGCATATAAGGAGACATTTACACTTAH |
| 17 (SEQ ID NO: 87) | VGGGTTATTGATTCTGTTGTGGGCAAGGAAGATGGACTTGG |
| | TGTGGAGAATCTACATGGAAGTGCTGCTATTGCCAGTGCTTA |
| | TTCTAGGGCATATAAGGAGACATTTACACTTAH |
| 18 (SEQ ID NO: 88) | VGGGTTATTGATTCTGTTGTGGGCAAGGAAGATGGACTTGG |
| | TGTGGAGAATCTGCATGGAAGTGCTGCTATTGCCAGTGCTTA |
| | TTCTAGGGCATATAAGGAGACATTTACACTTAH |
| 19 (SEQ ID NO: 89) | VGGGTTATTGATTCTGTTGTGGGCAAGGAAGATGGACTTGG |
| | TGTGGAGAATACUCATGGAAGTGCTGCTATTGCCAGTGCTT |
| | ATTCTAGGGCATATAAGGAGACATTTACACTTAH |
| 20 (SEQ ID NO: 90) | VGGGTTATTGATTCTGTTGTGGGCAAGGAAGATGGACTTGG |
| | TGTGGAGAATACCCATGGAAGTGCTGCTATTGCCAGTGCTT |
| | ATTCTAGGGCATATAAGGAGACATTTACACTTAH |
| 21 (SEQ ID NO: 91) | VGGGTTATTGATTCTGTTGTGGGCAAGGAAGATGGACTTGG |
| | TGTGGAGAATACACATGGAAGTGCTGCTATTGCCAGTGCTT |
| | ATTCTAGGGCATATAAGGAGACATTTACACTTAH |
| 22 (SEQ ID NO: 92) | VGGGTTATTGATTCTGTTGTGGGCAAGGAAGATGGACTTGG |
| | TGTGGAGAATACGCATGGAAGTGCTGCTATTGCCAGTGCTT |
| | ATTCTAGGGCATATAAGGAGACATTTACACTTAH |
| 23 (SEQ ID NO: 93) | VGGGTTATTGATTCTGTTGTGGGCAAGGAAGATGGACTTGG |
| | TGTGGAGAATCTTCATGGAAGTGGAGCTATTGCCAGTGCTT |
| | ATTCTAGGGCATATAAGGAGACATTTACACTTAH |
| 24 (SEQ ID NO: 94) | VGGGTTATTGATTCTGTTGTGGGCAAGGAAGATGGACTTGG |
| | TGTGGAGAATCTCCATGGAAGTGGGGCTATTGCCAGTGCTT |
| | ATTCTAGGGCATATAAGGAGACATTTACACTTAH |
| 25 (SEQ ID NO: 95) | VGGGTTATTGATTCTGTTGTGGGCAAGGAAGATGGACTTGG |
| | TGTGGAGAATCTACATGGAAGTGGTGCTATTGCCAGTGCTT |
| | ATTCTAGGGCATATAAGGAGACATTTACACTTAH |
| 26 (SEQ ID NO: 96) | VGGGTTATTGATTCTGTTGTGGGCAAGGAAGATGGACTTGG |
| | TGTGGAGAATCTGCATGGAAGTAACGCTATTGCCAGTGCTT |
| | ATTCTAGGGCATATAAGGAGACATTTACACTTAH |
| 27 (SEQ ID NO: 97) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGTATATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 28 (SEQ ID NO: 98) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGAAGATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 29 (SEQ ID NO: 99 | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGGAGATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 30 (SEQ ID NO: 100) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGTAGATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 31 (SEQ ID NO: 101) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGCAGATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 32 (SEQ ID NO: 102) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGAGTATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 33 (SEQ ID NO: 103) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGGGTATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 34 (SEQ ID NO: 104) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGTGTATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 35 (SEQ ID NO: 105) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCAGCACTTCCATGCGTATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 36 (SEQ ID NO: 106) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCTCCACTTCCATGAAGATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 37 (SEQ ID NO: 107) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCCCCACTTCCATGGAGATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 38 (SEQ ID NO: 108) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCACCACTTCCATGTAGATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 39 (SEQ ID NO: 109) | VGAGCAAGATAAGCTCCTATTCCAACAGTTCTTCCAGTCAC |
| | AAATGTAAGTGTAAATGTCTCCTTATATGCCCTAGAATAAGC |
| | ACTGGCAATAGCGCCACTTCCATGCAGATTCTCCACACCAA |
| | GTCCATCTTCCTTGCCCACAACAGAATCAATAACCCACCTAA |
| | TTTCACCACTGTCTAGCTGCATCTTATGTGCTATGACH |
| 40 (SEQ ID NO: 110) | VTAAGTGTAAATGTCTCCTTATATGCCCTAGAATAAGCACTG |
| | GCAATAGCAGCACTTCCATGTATATTCTCCACACCAAGTCCA |
| | TCTTCCTTGCCCACAACAGAATCAATAACCCH |
| 41 (SEQ ID NO: 111) | VTAAGTGTAAATGTCTCCTTATATGCCCTAGAATAAGCACTG |
| | GCAATAGCAGCACTTCCATGAAGATTCTCCACACCAAGTCC |
| | ATCTTCCTTGCCCACAACAGAATCAATAACCCH |
| 42 (SEQ ID NO: 112) | VTAAGTGTAAATGTCTCCTTATATGCCCTAGAATAAGCACTG |
| | GCAATAGCAGCACTTCCATGGAGATTCTCCACACCAAGTCC |
| | ATCTTCCTTGCCCACAACAGAATCAATAACCCH |
| 43 (SEQ ID NO: 113) | VTAAGTGTAAATGTCTCCTTATATGCCCTAGAATAAGCACTG |
| | GCAATAGCAGCACTTCCATGTAGATTCTCCACACCAAGTCC |
| | ATCTTCCTTGCCCACAACAGAATCAATAACCCH |
| 44 (SEQ ID NO: 114) | VTAAGTGTAAATGTCTCCTTATATGCCCTAGAATAAGCACTG |
| | GCAATAGCAGCACTTCCATGCAGATTCTCCACACCAAGTCC |
| | ATCTTCCTTGCCCACAACAGAATCAATAACCCH |
| 45 (SEQ ID NO: 115) | VTAAGTGTAAATGTCTCCTTATATGCCCTAGAATAAGCACTG |
| | GCAATAGCAGCACTTCCATGAGTATTCTCCACACCAAGTCC |
| | ATCTTCCTTGCCCACAACAGAATCAATAACCCH |
| 46 (SEQ ID NO: 116) | VTAAGTGTAAATGTCTCCTTATATGCCCTAGAATAAGCACTG |
| | GCAATAGCAGCACTTCCATGGGTATTCTCCACACCAAGTCC |
| | ATCTTCCTTGCCCACAACAGAATCAATAACCCH |
| 47 (SEQ ID NO: 117) | VTAAGTGTAAATGTCTCCTTATATGCCCTAGAATAAGCACTG |
| | GCAATAGCAGCACTTCCATGTGTATTCTCCACACCAAGTCCA |
| | TCTTCCTTGCCCACAACAGAATCAATAACCCH |
| 48 (SEQ ID NO: 118) | VTAAGTGTAAATGTCTCCTTATATGCCCTAGAATAAGCACTG |
| | GCAATAGCAGCACTTCCATGCGTATTCTCCACACCAAGTCC |
| | ATCTTCCTTGCCCACAACAGAATCAATAACCCH |
| 49 (SEQ ID NO: 119) | VTAAGTGTAAATGTCTCCTTATATGCCCTAGAATAAGCACTG |
| | GCAATAGCTCCACTTCCATGAAGATTCTCCACACCAAGTCC |
| | ATCTTCCTTGCCCACAACAGAATCAATAACCCH |
| 50 (SEQ ID NO: 120) | VTAAGTGTAAATGTCTCCTTATATGCCCTAGAATAAGCACTG |
| | GCAATAGCCCCACTTCCATGGAGATTCTCCACACCAAGTCC |
| | ATCTTCCTTGCCCACAACAGAATCAATAACCCH |
| 51 (SEQ ID NO: 121) | VTAAGTGTAAATGTCTCCTTATATGCCCTAGAATAAGCACTG |
| | GCAATAGCACCACTTCCATGTAGATTCTCCACACCAAGTCC |
| | ATCTTCCTTGCCCACAACAGAATCAATAACCCH |
| 52 (SEQ ID NO: 122) | VTAAGTGTAAATGTCTCCTTATATGCCCTAGAATAAGCACTG |
| | GCAATAGCGCCACTTCCATGCAGATTCTCCACACCAAGTCC |
| | ATCTTCCTTGCCCACAACAGAATCAATAACCCH |
| 53 (SEQ ID NO: 123) | VGATAAACCCAGACCGCATTGAGTGCTATGCTGAGAGGACT |
| | GCAAAAGGCAGTGCTCTCGAACCGCAAGGGTTAATTGAGAT |
| | CAAGTTCAGGTCAGAGGAACTCCH |
| 54 (SEQ ID NO: 124) | VGATAAACCCAGACCGCATTGAGTGCTATGCTGAGAGGACT |
| | GCAAAAGGCAGTGCTCTGGAACCGCAAGGGTTAATTGAGAT |
| | CAAGTTCAGGTCAGAGGAACTCCH |
| 55 (SEQ ID NO: 125) | VGGAGTTCCTCTGACCTGAACTTGATCTCAATTAACCCTTGC |
| | GGTTCGAGAGCACTGCCTTTTGCAGTCCTCTCAGCATAGCAC |
| | TCAATGCGGTCTGGGTTTATCH |
| 56 (SEQ ID NO: 126) | VGGAGTTCCTCTGACCTGAACTTGATCTCAATTAACCCTTGC |
| | GGTTCCAGAGCACTGCCTTTTGCAGTCCTCTCAGCATAGCAC |
| | TCAATGCGGTCTGGGTTTATCH |
| 57 (SEQ ID NO: 127) | VGATAAACCCAGACCGCATTGAGTGCTATGCTGAGAGGACT |
| | GCAAAAGGCAGTGGTCTCGAACCGCAAGGGTTAATTGAGAT |
| | CAAGTTCAGGTCAGAGGAACTCCH |
| 58 (SEQ ID NO: 128) | VGATAAACCCAGACCGCATTGAGTGCTATGCTGAGAGGACT |
| | GCAAAAGGCAGTGGTCTGGAACCGCAAGGGTTAATTGAGAT |
| | CAAGTTCAGGTCAGAGGAACTCCH |
| 59 (SEQ ID NO: 129) | VGGAGTTCCTCTGACCTGAACTTGATCTCAATTAACCCTTGC |
| | GGTTCGAGACCACTGCCTTTTGCAGTCCTCTCAGCATAGCAC |
| | TCAATGCGGTCTGGGTTTATCH |
| 60 (SEQ ID NO: 130) | VGGAGTTCCTCTGACCTGAACTTGATCTCAATTAACCCTTGC |
| | GGTTCCAGACCACTGCCTTTTGCAGTCCTCTCAGCATAGCAC |
| | TCAATGCGGTCTGGGTTTATCH |
| 61 (SEQ ID NO: 131) | VGATAAACCCAGACCGCATTGAGTGCTATGCTGAGAGGACT |
| | GCAAAAGGCAATGCTCTCGAACCGCAAGGGTTAATTGAGAT |
| | CAAGTTCAGGTCAGAGGAACTCCH |
| 62 (SEQ ID NO: 132) | VGATAAACCCAGACCGCATTGAGTGCTATGCTGAGAGGACT |
| | GCAAAAGGCAATGCTCTGGAACCGCAAGGGTTAATTGAGAT |
| | CAAGTTCAGGTCAGAGGAACTCCH |
| 63 (SEQ ID NO: 133) | VGGAGTTCCTCTGACCTGAACTTGATCTCAATTAACCCTTGC |
| | GGTTCGAGATCACTGCCTTTTGCAGTCCTCTCAGCATAGCAC |
| | TCAATGCGGTCTGGGTTTATCH |
| 64 (SEQ ID NO: 134) | VGGAGTTCCTCTGACCTGAACTTGATCTCAATTAACCCTTGC |
| | GGTTCCAGATCACTGCCTTTTGCAGTCCTCTCAGCATAGCAC |
| | TCAATGCGGTCTGGGTTTATCH |
| 65 (SEQ ID NO: 135) | VGATAAACCCAGACCGCATTGAGTGCTATGCTGAGAGGACT |
| | GCAAAAGGCAATGGTCTCGAACCGCAAGGGTTAATTGAGAT |
| | CAAGTTCAGGTCAGAGGAACTCCH |
| 66 (SEQ ID NO: 136) | VGATAAACCCAGACCGCATTGAGTGCTATGCTGAGAGGACT |
| | GCAAAAGGCAATGGTCTGGAACCGCAAGGGTTAATTGAGAT |
| | CAAGTTCAGGTCAGAGGAACTCCH |
| 67 (SEQ ID NO: 137) | VGGAGTTCCTCTGACCTGAACTTGATCTCAATTAACCCTTGC |
| | GGTTCGAGATCACTGCCTTTTGCAGTCCTCTCAGCATAGCAC |
| | TCAATGCGGTCTGGGTTTATCH |
| 68 (SEQ ID NO: 138) | VGGAGTTCCTCTGACCTGAACTTGATCTCAATTAACCCTTGC |
| | GGTTCCAGATCACTGCCTTTTGCAGTCCTCTCAGCATAGCAC |
| | TCAATGCGGTCTGGGTTTATCH |
| 69 (SEQ ID NO: 139) | VGGCTCGACTATTGTTGAGAACCTTAGGACATACAATCAGC |
| | CTGCCTTTGTCTACATTCCCATGGCTGCAGAGCTACGAGGAG |
| | GGGCTTGGGTTGTGGTTGATAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAGH |
| 70 (SEQ ID NO: 140) | VGGCTCGACTATTGTTGAGAACCTTAGGACATACAATCAGC |
| | CTGCCTTTGTCTACATTCCCATGGCTGCAGAGCTACGAGGAG |
| | GGGCTTGGGTTGTGGTTGGAAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAGH |
| 71 (SEQ ID NO: 141) | VGGCTCGACTATTGTTGAGAACCTTAGGACATACAATCAGC |
| | CTGCCTTTGTCTACATTCCCATGGCTGCAGAGCTACGAGGAG |
| | GGGCTTGGGTTGTCGTTGATAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAGH |
| 72 (SEQ ID NO: 142) | VGGCTCGACTATTGTTGAGAACCTTAGGACATACAATCAGC |
| | CTGCCTTTGTCTACATTCCCATGGCTGCAGAGCTACGAGGAG |
| | GGGCTTGGGTTGTCGTTGGAAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAGH |
| 73 (SEQ ID NO: 143) | VGGCTCGACTATTGTTGAGAACCTTAGGACATACAATCAGC |
| | CTGCCTTTGTCTACATTCCCATGGCTGCAGAGCTACGAGGAG |
| | GCGCTTGGGTTGTGGTTGATAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAGH |
| 74 (SEQ ID NO: 144) | VGGCTCGACTATTGTTGAGAACCTTAGGACATACAATCAGC |
| | CTGCCTTTGTCTACATTCCCATGGCTGCAGAGCTACGAGGAG |
| | GCGCTTGGGTTGTGGTTGGAAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAGH |
| 75 (SEQ ID NO: 145) | VCTGACCTGAACTTGATCTCAATTAACCCTTGCGGTTCCAGA |
| | ACATTGCCTTTTGCAGTCCTCTCAGCATAGCACTCAATGCGG |
| | TCTGGGTTTATCTTGCTATCAACCACAACCCAAGCCCCTCCT |
| | CGTAGCTCTGCAGCCATGGGAATGTAGACAAAGGCAGGCTG |
| | ATTGTATGTCCTAAGGTTCTCAACAATAGTCGAGCCH |
| 76 (SEQ ID NO: 146) | VCTGACCTGAACTTGATCTCAATTAACCCTTGCGGTTCCAGA |
| | ACATTGCCTTTTGCAGTCCTCTCAGCATAGCACTCAATGCGG |
| | TCTGGGTTTATCTTGCTTCCAACCACAACCCAAGCCCCTCCT |
| | CGTAGCTCTGCAGCCATGGGAATGTAGACAAAGGCAGGCTG |
| | ATTGTATGTCCTAAGGTTCTCAACAATAGTCGAGCCH |
| 77 (SEQ ID NO: 147) | VCTGACCTGAACTTGATCTCAATTAACCCTTGCGGTTCCAGA |
| | ACATTGCCTTTTGCAGTCCTCTCAGCATAGCACTCAATGCGG |
| | TCTGGGTTTATCTTGCTATCAACGACAACCCAAGCCCCTCCT |
| | CGTAGCTCTGCAGCCATGGGAATGTAGACAAAGGCAGGCTG |
| | ATTGTATGTCCTAAGGTTCTCAACAATAGTCGAGCCH |
| 78 (SEQ ID NO: 148) | VCTGACCTGAACTTGATCTCAATTAACCCTTGCGGTTCCAGA |
| | ACATTGCCTTTTGCAGTCCTCTCAGCATAGCACTCAATGCGG |
| | TCTGGGTTTATCTTGCTTCCAACGACAACCCAAGCCCCTCCT |
| | CGTAGCTCTGCAGCCATGGGAATGTAGACAAAGGCAGGCTG |
| | ATTGTATGTCCTAAGGTTCTCAACAATAGTCGAGCCH |
| 79 (SEQ ID NO: 149) | VCTGACCTGAACTTGATCTCAATTAACCCTTGCGGTTCCAGA |
| | ACATTGCCTTTTGCAGTCCTCTCAGCATAGCACTCAATGCGG |
| | TCTGGGTTTATCTTGCTATCAACCACAACCCAAGCGCCTCCT |
| | CGTAGCTCTGCAGCCATGGGAATGTAGACAAAGGCAGGCTG |
| | ATTGTATGTCCTAAGGTTCTCAACAATAGTCGAGCCH |
| 80 (SEQ ID NO: 150) | VCTGACCTGAACTTGATCTCAATTAACCCTTGCGGTTCCAGA |
| | ACATTGCCTTTTGCAGTCCTCTCAGCATAGCACTCAATGCGG |
| | TCTGGGTTTATCTTGCTTCCAACCACAACCCAAGCGCCTCCT |
| | CGTAGCTCTGCAGCCATGGGAATGTAGACAAAGGCAGGCTG |
| | ATTGTATGTCCTAAGGTTCTCAACAATAGTCGAGCCH |
| 81 (SEQ ID NO: 151) | VTCTCAGCATAGCACTCAATGCGGTCTGGGTTTATCTTGCTT |
| | CCAACGACAACCCAAGCCCCTCCTCGTAGCTCTGCAGCCAT |
| | GGGAATGTAH |
| 82 (SEQ ID NO: 152) | VTCTCAGCATAGCACTCAATGCGGTCTGGGTTTATCTTGCTT |
| | CCAACGACAACCCAAGCCCCTCCTCGTAGCTCTGCAGCCAT |
| | GGGAATGTAH |
| 83 (SEQ ID NO: 153) | VTTGCAGTCCTCTCAGCATAGCACTCAATGCGGTCTGGGTTT |
| | ATCTTGCTTCCAACGACAACCCAAGCCCCTCCTCGTAGCTCT |
| | GCAGCCATGGGAATGTAH |
| 84 (SEQ ID NO: 154) | VTTGCAGTCCTCTCAGCATAGCACTCAATGCGGTCTGGGTTT |
| | ATCTTGCTTCCAACGACAACCCAAGCCCCTCCTCGTAGCTCT |
| | GCAGCCATGGGAATGTAH |
| 85 (SEQ ID NO: 155) | VGCTCGACTATTGTTGAGAACCTTAGGACATACAATCAGCC |
| | TGCCTTTGTCTACATTCCCATGGCTGCAGAGCTACGAGGAGG |
| | GGCTTGGGTTGTCGTTGGTAGCAAGATAAACCCAGACCGCA |
| | TTGAGTGCTATGCTGAGAGGACTGCAAAAGGCAATGTTCTG |
| | GAACCGCAAGGGTTAATTGAGATCAAGTTCAGGTCAGH |
| 86 (SEQ ID NO: 156) | VTACATTCCCATGGCTGCAGAGCTACGAGGAGGGGCTTGGG |
| | TTGTCGTTGGTAGCAAGATAAACCCAGACCGCATTGAGTGC |
| | TATGCTGAGAGGACTGCAAH |
| 87 (SEQ ID NO: 157) | VTACATTCCCATGGCTGCAGAGCTACGAGGAGGGGCTTGGG |
| | TTGTCGTTGGTAGCAAGATAAACCCAGACCGCATTGAGAAC |
| | TATGCTGAGAGGACTGCAAH |
| 88 (SEQ ID NO: 158) | VTACATTCCCATGGCTGCAGAGCTACGAGGAGGGGCTTGGG |
| | TTGTCGTTGATAGCAAGATAAACCCAGACCGCATTGAGAAC |
| | TATGCTGAGAGGACTGCAAH |
| 89 (SEQ ID NO: 159) | VTACATTCCCATGGCTGCAGAGCTACGAGGAGGGGCTTGGC |
| | TTGTCGTTGATAGCAAGATAAACCCAGACCGCATTGAGTGC |
| | TATGCTGAGAGGACTGCAAH |
| 90 (SEQ ID NO: 160) | VTGACCTGAACTTGATCTCAATTAACCCTTGCGGTTCCAGAA |
| | CATTGCCTTTTGCAGTCCTCTCAGCATAGCACTCAATGCGGT |
| | CTGGGTTTATCTTGCTACCAACGACAACCCAAGCCCCTCCTC |
| | GTAGCTCTGCAGCCATGGGAATGTAGACAAAGGCAGGCTGA |
| | TTGTATGTCCTAAGGTTCTCAACAATAGTCGAGCCH |
| 91 (SEQ ID NO: 161) | VTGCAGTCCTCTCAGCATAGCACTCAATGCGGTCTGGGTTTA |
| | TCTTGCTACCAACGACAACCCAAGCCCCTCCTCGTAGCTCTG |
| | CAGCCATGGGAATGTAGH |
| 92 (SEQ ID NO: 162) | VTGCAGTCCTCTCAGCATAGTTCTCAATGCGGTCTGGGTTTA |
| | TCTTGCTACCAACGACAACCCAAGCCCCTCCTCGTAGCTCTG |
| | CAGCCATGGGAATGTAGH |
| 93 (SEQ ID NO: 163) | VTTGCAGTCCTCTCAGCATAGTTCTCAATGCGGTCTGGGTTT |
| | ATCTTGCTATCAACGACAACCCAAGCCCCTCCTCGTAGCTCT |
| | GCAGCCATGGGAATGTAGH |
| 94 (SEQ ID NO: 164) | VTGCAGTCCTCTCAGCATAGCACTCAATGCGGTCTGGGTTTA |
| | TCTTGCTATCAACGACAAGCCAAGCCCCTCCTCGTAGCTCTG |
| | CAGCCATGGGAATGTAGH |
| 95 (SEQ ID NO: 165) | VCTGACCTGAACTTGATCTCAATTAACCCTTGCGGTTCCAGA |
| | ACATTGCCTTTTGCAGTCCTCTCAGCATAGCACTCAATGCGG |
| | TCTGGGTTTATCTTGCTTCCAACGACAACCCAAGCCCCTCCT |
| | CGTAGCTCTGCAGCCATGGGAATGTAGACAAAGGCAGGCTG |
| | ATTGTATGTCCTAAGGTTCTCAACAATAGTCGAGCCH |
| 96 (SEQ ID NO: 166) | VTTTGCAGTCCTCTCAGCATAGCACTCAATGCGGTCTGGGTT |
| | TATCTTGCTTCCAACGACAACCCAAGCCCCTCCTCGTAGCTC |
| | TGCAGCCATGGGAATGTAH |
| 97 (SEQ ID NO: 167) | VTGCACTGTGGATATCTACCGTGAGATGGAAGATAAAGAAT |
| | CACGGAAGTTAGTATACCATCCCGCCACTCCGGCGGCTGGT |
| | CCTCTGCATGGTGTGGCAH |
| 98 (SEQ ID NO: 168) | VTGCACTGTGGATATCTACCGTGAGATGGAAGATAAAGAAT |
| | CACGGAAGGTTGTATACCATCCCGCCACTCCGGCGGCTGGT |
| | CCTCTGCATGGTGTGGCAH |
| 99 (SEQ ID NO: 169) | VTGCACTGTGGATATCTACCGTGAGATGGAAGATAAAGAAT |
| | CACGGAAGGTCGTATACCATCCCGCCACTCCGGCGGCTGGT |
| | CCTCTGCATGGTGTGGCAH |
| 100 (SEQ ID NO: 170) | VTGCACTGTGGATATCTACCGTGAGATGGAAGATAAAGAAT |
| | CACGGAAGGTAGTATACCATCCCGCCACTCCGGCGGCTGGT |
| | CCTCTGCATGGTGTGGCAH |
| 101 (SEQ ID NO: 171) | VTGCACTGTGGATATCTACCGTGAGATGGAAGATAAAGAAT |
| | CACGGAAGGTGGTATACCATCCCGCCACTCCGGCGGCTGGT |
| | CCTCTGCATGGTGTGGCAH |
| 102 (SEQ ID NO: 172) | VTGCACTGTGGATATCTACCGTGAGATGGAAGATAAAGAAT |
| | CACGGAAGTTTGTATACCATCCCGCCACTCCGGCGGCTGGT |
| | CCTCTGCATGGTGTGGCAH |
| 103 (SEQ ID NO: 173) | VTGCACTGTGGATATCTACCGTGAGATGGAAGATAAAGAAT |
| | CACGGAAGTTCGTATACCATCCCGCCACTCCGGCGGCTGGT |
| | CCTCTGCATGGTGTGGCAH |
| 104 (SEQ ID NO: 174) | VTGCACTGTGGATATCTACCGTGAGATGGAAGATAAAGAAT |
| | CACGCAAGGTTGTATACCATCCCGCCACTCCGGCGGCTGGT |
| | CCTCTGCATGGTGTGGCAH |
| 105 (SEQ ID NO: 175) | VTGCACTGTGGATATCTACCGTGAGATGGAAGATAAAGAAT |
| | CACGCAAGGTCGTATACCATCCCGCCACTCCGGCGGCTGGT |
| | CCTCTGCATGGTGTGGCAH |
| 106 (SEQ ID NO: 176) | VTGCACTGTGGATATCTACCGTGAGATGGAAGATAAAGAAT |
| | CACGCAAGGTAGTATACCATCCCGCCACTCCGGCGGCTGGT |
| | CCTCTGCATGGTGTGGCAH |
| 107 (SEQ ID NO: 177) | VTGCACTGTGGATATCTACCGTGAGATGGAAGATAAAGAAT |
| | CACGCAAGGTGGTATACCATCCCGCCACTCCGGCGGCTGGT |
| | CCTCTGCATGGTGTGGCAH |
| 108 (SEQ ID NO: 178) | VTGCACTGTGGATATCTACCGTGAGATGGAAGATAAAGAAT |
| | CACGCAAGTTTGTATACCATCCCGCCACTCCGGCGGCTGGTC |
| | CTCTGCATGGTGTGGCAH |
| 109 (SEQ ID NO: 179) | VTGCACTGTGGATATCTACCGTGAGATGGAAGATAAAGAAT |
| | CACGCAAGTTCGTATACCATCCCGCCACTCCGGCGGCTGGT |
| | CCTCTGCATGGTGTGGCAH |
| 110 (SEQ ID NO: 180) | VTGCACTGTGGATATCTACCGTGAGATGGAAGATAAAGAAT |
| | CACGGAAGGTTGTATATCATCCCGCCACTCCGGCGGCTGGT |
| | CCTCTGCATGGTGTGGCAH |
| 111 (SEQ ID NO: 181) | VTGCACTGTGGATATCTACCGTGAGATGGAAGATAAAGAAT |
| | CACGGAAGGTCGTATATCATCCCGCCACTCCGGCGGCTGGT |
| | CCTCTGCATGGTGTGGCAH |
| 112 (SEQ ID NO: 182) | VTGCACTGTGGATATCTACCGTGAGATGGAAGATAAAGAAT |
| | CACGGAAGGTAGTATATCATCCCGCCACTCCGGCGGCTGGT |
| | CCTCTGCATGGTGTGGCAH |
| 113 (SEQ ID NO: 183) | VTGCACTGTGGATATCTACCGTGAGATGGAAGATAAAGAAT |
| | CACGGAAGGTGGTATATCATCCCGCCACTCCGGCGGCTGGT |
| | CCTCTGCATGGTGTGGCAH |
| 114 (SEQ ID NO: 184) | VTGCACTGTGGATATCTACCGTGAGATGGAAGATAAAGAAT |
| | CACGGAAGTTTGTATATCATCCCGCCACTCCGGCGGCTGGTC |
| | CTCTGCATGGTGTGGCAH |
| 115 (SEQ ID NO: 185) | VTGCACTGTGGATATCTACCGTGAGATGGAAGATAAAGAAT |
| | CACGGAAGTTCGTATATCATCCCGCCACTCCGGCGGCTGGT |
| | CCTCTGCATGGTGTGGCAH |
| 116 (SEQ ID NO: 186) | VTTTCCTAGTGGTAGGGAGATTATTGTTGTTGCAAATGATAT |
| | TACGTTCAGAGCTGGATCATTTGGCCCAAGGGAAGATGCAT |
| | TTTTTGAAGCTGTTACCH |
| 117 (SEQ ID NO: 187) | VTTTCCTAGTGGTAGGGAGATTATTGTTGTTGCAAATGATAT |
| | TACGTTCATTGCTGGATCATTTGGCCCAAGGGAAGATGCATT |
| | TTTTGAAGCTGTTACCH |
| 118 (SEQ ID NO: 188) | VTTTCCTAGTGGTAGGGAGATTATTGTTGTTGCAAATGATAT |
| | TACGTTCATCGCTGGATCATTTGGCCCAAGGGAAGATGCATT |
| | TTTTGAAGCTGTTACCH |
| 119 (SEQ ID NO: 189) | VTTTCCTAGTGGTAGGGAGATTATTGTTGTTGCAAATGATAT |
| | TACGTTCATAGCTGGATCATTTGGCCCAAGGGAAGATGCAT |
| | TTTTTGAAGCTGTTACCH |
| 120 (SEQ ID NO: 190) | VTTTCCTAGTGGTAGGGAGATTATTGTTGTTGCAAATGATAT |
| | TACGTTCATTGCTGGTTCTTTCGGCCCAAGGGAAGATGCATT |
| | TTTTGAAGCTGTTACCH |
| 121 (SEQ ID NO: 191) | VTTTCCTAGTGGTAGGGAGATTATTGTTGTTGCAAATGATAT |
| | TACGTTCATCGCTGGTTCTTTCGGCCCAAGGGAAGATGCATT |
| | TTTTGAAGCTGTTACCH |
| 122 (SEQ ID NO: 192) | VTTTCCTAGTGGTAGGGAGATTATTGTTGTTGCAAATGATAT |
| | TACGTTCATAGCTGGTTCTTTCGGCCCAAGGGAAGATGCATT |
| | TTTTGAAGCTGTTACCH |
| 123 (SEQ ID NO: 193) | VAAAGCTTGGTGGAATTCCAGTGGGTGTGATAGCTGTGGAG |
| | ACTCAGACCATGATGCAAACTATCCCTGCTGACCCTGGTCA |
| | GCTTGATTCCCGTGAGCAATH |
| 124 (SEQ ID NO: 194) | VAAAGCTTGGTGGAATTCCAGTGGGTGTGATAGCTGTGGAG |
| | ACTCAGACCACTATGCAAACTATCCCTGCTGACCCTGGTCA |
| | GCTTGATTCCCGTGAGCAATH |
| 125 (SEQ ID NO: 195) | VAAAGCTTGGTGGAATTCCAGTGGGTGTGATAGCTGTGGAG |
| | ACTCAGACCACCATGCAAACTATCCCTGCTGACCCTGGTCA |
| | GCTTGATTCCCGTGAGCAATH |
| 126 (SEQ ID NO: 196) | VAAAGCTTGGTGGAATTCCAGTGGGTGTGATAGCTGTGGAG |
| | ACTCAGACCACAATGCAAACTATCCCTGCTGACCCTGGTCA |
| | GCTTGATTCCCGTGAGCAATH |
| 127 (SEQ ID NO: 197) | VAAAGCTTGGTGGAATTCCAGTGGGTGTGATAGCTGTGGAG |
| | ACTCAGACCACGATGCAAACTATCCCTGCTGACCCTGGTCA |
| | GCTTGATTCCCGTGAGCAATH |
| 128 (SEQ ID NO: 198) | VAAAGCTTGGTGGAATTCCAGTGGGTGTGATAGCTGTGGAG |
| | ACTCAGACGACTATGCAAACTATCCCTGCTGACCCTGGTCA |
| | GCTTGATTCCCGTGAGCAATH |
| 129 (SEQ ID NO: 199) | VAAAGCTTGGTGGAATTCCAGTGGGTGTGATAGCTGTGGAG |
| | ACTCAGACGACCATGCAAACTATCCCTGCTGACCCTGGTCA |
| | GCTTGATTCCCGTGAGCAATH |
| 130 (SEQ ID NO: 200) | VAAAGCTTGGTGGAATTCCAGTGGGTGTGATAGCTGTGGAG |
| | ACTCAGACGACAATGCAAACTATCCCTGCTGACCCTGGTCA |
| | GCTTGATTCCCGTGAGCAATH |
| 131 (SEQ ID NO: 201) | VAAAGCTTGGTGGAATTCCAGTGGGTGTGATAGCTGTGGAG |
| | ACTCAGACGACGATGCAAACTATCCCTGCTGACCCTGGTCA |
| | GCTTGATTCCCGTGAGCAATH |
| 132 (SEQ ID NO: 202) | VAAAGCTTGGTGGAATTCCAGTGGGTGTGATAGCTGTCGAG |
| | ACTCAGACCACTATGCAAACTATCCCTGCTGACCCTGGTCA |
| | GCTTGATTCCCGTGAGCAATH |
| 133 (SEQ ID NO: 203) | VAAAGCTTGGTGGAATTCCAGTGGGTGTGATAGCTGTCGAG |
| | ACTCAGACCACCATGCAAACTATCCCTGCTGACCCTGGTCA |
| | GCTTGATTCCCGTGAGCAATH |
| 134 (SEQ ID NO: 204) | VAAAGCTTGGTGGAATTCCAGTGGGTGTGATAGCTGTCGAG |
| | ACTCAGACCACAATGCAAACTATCCCTGCTGACCCTGGTCA |
| | GCTTGATTCCCGTGAGCAATH |
| 135 (SEQ ID NO: 205) | VAAAGCTTGGTGGAATTCCAGTGGGTGTGATAGCTGTCGAG |
| | ACTCAGACCACGATGCAAACTATCCCTGCTGACCCTGGTCA |
| | GCTTGATTCCCGTGAGCAATH |
| 136 (SEQ ID NO: 206) | VTTCTGTTGTGGGCAAGGAAGATGGACTTGGTGTGGAGAAT |
| | ATACATGGAAGTGCTGCTATTGCCAGTGCTTATTCTAGGGCA |
| | TATAAGGAGACATTTACACH |
| 137 (SEQ ID NO: 207) | VTTCTGTTGTGGGCAAGGAAGATGGACTTGGTGTGGAGAAT |
| | ATACATGGAAATGCTGCTATTGCCAGTGCTTATTCTAGGGCA |
| | TATAAGGAGACATTTACACH |
| 138 (SEQ ID NO: 208) | VTTCTGTTGTGGGCAAGGAAGATGGACTTGGTGTGGAGAAT |
| | ATACATGGAAACGCTGCTATTGCCAGTGCTTATTCTAGGGCA |
| | TATAAGGAGACATTTACACH |
| 139 (SEQ ID NO: 209) | VTTCTGTTGTGGGCAAGGAAGATGGACTTGGTGTGGAGAAC |
| | ATTCATGGAAATGCTGCTATTGCCAGTGCTTATTCTAGGGCA |
| | TATAAGGAGACATTTACACH |
| 140 (SEQ ID NO: 210) | VTTCTGTTGTGGGCAAGGAAGATGGACTTGGTGTGGAGAAC |
| | ATTCATGGAAACGCTGCTATTGCCAGTGCTTATTCTAGGGCA |
| | TATAAGGAGACATTTACACH |
| 141 (SEQ ID NO: 211) | VTTCTGTTGTGGGCAAGGAAGATGGACTTGGTGTGGAGAAT |
| | ATACATGGAAATGCTGCTATTGCGAGTGCTTATTCTAGGGCA |
| | TATAAGGAGACATTTACACH |
| 142 (SEQ ID NO: 212) | VTTCTGTTGTGGGCAAGGAAGATGGACTTGGTGTGGAGAAT |
| | ATACATGGAAACGCTGCTATTGCGAGTGCTTATTCTAGGGC |
| | ATATAAGGAGACATTTACACH |
| 143 (SEQ ID NO: 213) | VTACATTTGTGACTGGAAGAACTGTTGGAATAGGAGCTTAT |
| | CTTGCTCGACTTGGCATCCGGTGCATACAGCGTCTTGACCAG |
| | CCTATTATTCTTACAGGCTH |
| 144 (SEQ ID NO: 214) | VTACATTTGTGACTGGAAGAACTGTTGGAATAGGAGCTTAT |
| | CTTGCTCGACATGGCATCCGGTGCATACAGCGTCTTGACCA |
| | GCCTATTATTCTTACAGGCTH |
| 145 (SEQ ID NO: 215) | VTACATTTGTGACTGGAAGAACTGTTGGAATAGGAGCTTAT |
| | CTTGCTCGACACGGCATCCGGTGCATACAGCGTCTTGACCA |
| | GCCTATTATTCTTACAGGCTH |
| 146 (SEQ ID NO: 216) | VTACATTTGTGACTGGAAGAACTGTTGGAATAGGAGCTTAT |
| | CTTGCTCGTCATGGCATCCGGTGCATACAGCGTCTTGACCAG |
| | CCTATTATTCTTACAGGCTH |
| 147 (SEQ ID NO: 217) | VTACATTTGTGACTGGAAGAACTGTTGGAATAGGAGCTTAT |
| | CTTGCTCGTCACGGCATCCGGTGCATACAGCGTCTTGACCAG |
| | CCTATTATTCTTACAGGCTH |
| 148 (SEQ ID NO: 218) | VTACATTTGTGACTGGAAGAACTGTTGGAATAGGAGCTTAT |
| | CTTGCTCGACATGGCATACGGTGCATACAGCGTCTTGACCA |
| | GCCTATTATTCTTACAGGCTH |
| 149 (SEQ ID NO: 219) | VTACATTTGTGACTGGAAGAACTGTTGGAATAGGAGCTTAT |
| | CTTGCTCGACACGGCATACGGTGCATACAGCGTCTTGACCA |
| | GCCTATTATTCTTACAGGCTH |
| 150 (SEQ ID NO: 220) | VTACATTTGTGACTGGAAGAACTGTTGGAATAGGAGCTTAT |
| | CTTGCTCGACATGGCATCCGCTGCATACAGCGTCTTGACCAG |
| | CCTATTATTCTTACAGGCTH |
| 151 (SEQ ID NO: 221) | VTACATTTGTGACTGGAAGAACTGTTGGAATAGGAGCTTAT |
| | CTTGCTCGACACGGCATCCGCTGCATACAGCGTCTTGACCA |
| | GCCTATTATTCTTACAGGCTH |
| 152 (SEQ ID NO: 222) | VTGTTGCAAATGATATTACGTTCAGAGCTGGATCATTTGGCC |
| | CAAGGGAAGATGCATTTTTTGAAGCTGTTACCAACCTAGCCT |
| | GTGAGAAGAAACTTCCTCH |
| 153 (SEQ ID NO: 223) | VTGTTGCAAATGATATTACGTTCAGAGCTGGATCATTTGGCC |
| | CAAGGGAAGAAGCATTTTTTGAAGCTGTTACCAACCTAGCC |
| | TGTGAGAAGAAACTTCCTCH |
| 154 (SEQ ID NO: 224) | VTGTTGCAAATGATATTACGTTCAGAGCTGGATCATTTGGCC |
| | CAAGGGAAGAGGCATTTTTTGAAGCTGTTACCAACCTAGCC |
| | TGTGAGAAGAAACTTCCTCH |
| 155 (SEQ ID NO: 225) | VTGTTGCAAATGATATTACGTTCAGAGCTGGATCATTTGGCC |
| | CAAGGGAAGGAGCATTTTTTGAAGCTGTTACCAACCTAGCC |
| | TGTGAGAAGAAACTTCCTCH |
| 156 (SEQ ID NO: 226) | VTGTTGCAAATGATATTACGTTCAGAGCTGGATCATTTGGCC |
| | CAAGGGAAGGTGCATTTTTTGAAGCTGTTACCAACCTAGCCT |
| | GTGAGAAGAAACTTCCTCH |
| 157 (SEQ ID NO: 227) | VTGTTGCAAATGATATTACGTTCAGAGCTGGATCATTTGGCC |
| | CAAGGGAAGGCGCATTTTTTGAAGCTGTTACCAACCTAGCC |
| | TGTGAGAAGAAACTTCCTCH |
| 158 (SEQ ID NO: 228) | VTGTTGCAAATGATATTACGTTCAGAGCTGGATCATTTGGCC |
| | CAAGGGAAGGGGCATTTTTTGAAGCTGTTACCAACCTAGCC |
| | TGTGAGAAGAAACTTCCTCH |
| 159 (SEQ ID NO: 229) | VTGTTGCAAATGATATTACGTTCAGAGCTGGATCATTTGGCC |
| | CAAGAGAAGAAGCATTTTTTGAAGCTGTTACCAACCTAGCC |
| | TGTGAGAAGAAACTTCCTCH |
| 160 (SEQ ID NO: 230) | VTGTTGCAAATGATATTACGTTCAGAGCTGGATCATTTGGCC |
| | CAAGAGAAGAGGCATTTTTTGAAGCTGTTACCAACCTAGCC |
| | TGTGAGAAGAAACTTCCTCH |
| 161 (SEQ ID NO: 231) | VTGTTGCAAATGATATTACGTTCAGAGCTGGATCATTTGGCC |
| | CAAGAGAAGGAGCATTTTTTGAAGCTGTTACCAACCTAGCC |
| | TGTGAGAAGAAACTTCCTCH |
| 162 (SEQ ID NO: 232) | VTGTTGCAAATGATATTACGTTCAGAGCTGGATCATTTGGCC |
| | CAAGAGAAGGTGCATTTTTTGAAGCTGTTACCAACCTAGCCT |
| | GTGAGAAGAAACTTCCTCH |
| 163 (SEQ ID NO: 233) | VTGTTGCAAATGATATTACGTTCAGAGCTGGATCATTTGGCC |
| | CAAGAGAAGGCGCATTTTTTGAAGCTGTTACCAACCTAGCC |
| | TGTGAGAAGAAACTTCCTCH |
| 164 (SEQ ID NO: 234) | VTGTTGCAAATGATATTACGTTCAGAGCTGGATCATTTGGCC |
| | CAAGAGAAGGGGCATTTTTTGAAGCTGTTACCAACCTAGCC |
| | TGTGAGAAGAAACTTCCTCH |
| 165 (SEQ ID NO: 235) | VTGTTGCAAATGATATTACGTTCAGAGCTGGATCATTTGGCC |
| | CTAGGGAAGAAGCATTTTTTGAAGCTGTTACCAACCTAGCCT |
| | GTGAGAAGAAACTTCCTCH |
| 166 (SEQ ID NO: 236) | VTGTTGCAAATGATATTACGTTCAGAGCTGGATCATTTGGCC |
| | CTAGGGAAGAGGCATTTTTTGAAGCTGTTACCAACCTAGCCT |
| | GTGAGAAGAAACTTCCTCH |
| 167 (SEQ ID NO: 237) | VTGTTGCAAATGATATTACGTTCAGAGCTGGATCATTTGGCC |
| | CTAGGGAAGGAGCATTTTTTGAAGCTGTTACCAACCTAGCCT |
| | GTGAGAAGAAACTTCCTCH |
| 168 (SEQ ID NO: 238) | VTGTTGCAAATGATATTACGTTCAGAGCTGGATCATTTGGCC |
| | CTAGGGAAGGTGCATTTTTTGAAGCTGTTACCAACCTAGCCT |
| | GTGAGAAGAAACTTCCTCH |
| 169 (SEQ ID NO: 239) | VTGTTGCAAATGATATTACGTTCAGAGCTGGATCATTTGGCC |
| | CTAGGGAAGGCGCATTTTTTGAAGCTGTTACCAACCTAGCCT |
| | GTGAGAAGAAACTTCCTCH |
| 170 (SEQ ID NO: 240) | VTGTTGCAAATGATATTACGTTCAGAGCTGGATCATTTGGCC |
| | CTAGGGAAGGGGCATTTTTTGAAGCTGTTACCAACCTAGCCT |
| | GTGAGAAGAAACTTCCTCH |
| 171 (SEQ ID NO: 241) | VTGCCACACCATGCAGAGGACCAGCCGCCGGAGTGGCGGG |
| | ATGGTATACTAACTTCCGTGATTCTTTATCTTCCATCTCACG |
| | GTAGATATCCACAGTGCAH |
| 172 (SEQ ID NO: 242) | VTGCCACACCATGCAGAGGACCAGCCGCCGGAGTGGCGGG |
| | ATGGTATACAACCTTCCGTGATTCTTTATCTTCCATCTCACG |
| | GTAGATATCCACAGTGCAH |
| 173 (SEQ ID NO: 243) | VTGCCACACCATGCAGAGGACCAGCCGCCGGAGTGGCGGG |
| | ATGGTATACGACCTTCCGTGATTCTTTATCTTCCATCTCACG |
| | GTAGATATCCACAGTGCAH |
| 174 (SEQ ID NO: 244) | VTGCCACACCATGCAGAGGACCAGCCGCCGGAGTGGCGGG |
| | ATGGTATACTACCTTCCGTGATTCTTTATCTTCCATCTCACGG |
| | TAGATATCCACAGTGCAH |
| 175 (SEQ ID NO: 245) | VTGCCACACCATGCAGAGGACCAGCCGCCGGAGTGGCGGG |
| | ATGGTATACCACCTTCCGTGATTCTTTATCTTCCATCTCACG |
| | GTAGATATCCACAGTGCAH |
| 176 (SEQ ID NO: 246) | VTGCCACACCATGCAGAGGACCAGCCGCCGGAGTGGCGGG |
| | ATGGTATACAAACTTCCGTGATTCTTTATCTTCCATCTCACG |
| | GTAGATATCCACAGTGCAH |
| 177 (SEQ ID NO: 247) | VTGCCACACCATGCAGAGGACCAGCCGCCGGAGTGGCGGG |
| | ATGGTATACGAACTTCCGTGATTCTTTATCTTCCATCTCACG |
| | GTAGATATCCACAGTGCAH |
| 178 (SEQ ID NO: 248) | VTGCCACACCATGCAGAGGACCAGCCGCCGGAGTGGCGGG |
| | ATGGTATACAACCTTGCGTGATTCTTTATCTTCCATCTCACG |
| | GTAGATATCCACAGTGCAH |
| 179 (SEQ ID NO: 249) | VTGCCACACCATGCAGAGGACCAGCCGCCGGAGTGGCGGG |
| | ATGGTATACGACCTTGCGTGATTCTTTATCTTCCATCTCACG |
| | GTAGATATCCACAGTGCAH |
| 180 (SEQ ID NO: 250) | VTGCCACACCATGCAGAGGACCAGCCGCCGGAGTGGCGGG |
| | ATGGTATACTACCTTGCGTGATTCTTTATCTTCCATCTCACG |
| | GTAGATATCCACAGTGCAH |
| 181 (SEQ ID NO: 251) | VTGCCACACCATGCAGAGGACCAGCCGCCGGAGTGGCGGG |
| | ATGGTATACCACCTTGCGTGATTCTTTATCTTCCATCTCACG |
| | GTAGATATCCACAGTGCAH |
| 182 (SEQ ID NO: 252) | VTGCCACACCATGCAGAGGACCAGCCGCCGGAGTGGCGGG |
| | ATGGTATACAAACTTGCGTGATTCTTTATCTTCCATCTCACG |
| | GTAGATATCCACAGTGCAH |
| 183 (SEQ ID NO: 253) | VTGCCACACCATGCAGAGGACCAGCCGCCGGAGTGGCGGG |
| | ATGGTATACGAACTTGCGTGATTCTTTATCTTCCATCTCACG |
| | GTAGATATCCACAGTGCAH |
| 184 (SEQ ID NO: 254) | VTGCCACACCATGCAGAGGACCAGCCGCCGGAGTGGCGGG |
| | ATGATATACAACCTTCCGTGATTCTTTATCTTCCATCTCACG |
| | GTAGATATCCACAGTGCAH |
| 185 (SEQ ID NO: 255) | VTGCCACACCATGCAGAGGACCAGCCGCCGGAGTGGCGGG |
| | ATGATATACGACCTTCCGTGATTCTTTATCTTCCATCTCACG |
| | GTAGATATCCACAGTGCAH |
| 186 (SEQ ID NO: 256) | VTGCCACACCATGCAGAGGACCAGCCGCCGGAGTGGCGGG |
| | ATGATATACTACCTTCCGTGATTCTTTATCTTCCATCTCACGG |
| | TAGATATCCACAGTGCAH |
| 187 (SEQ ID NO: 257) | VTGCCACACCATGCAGAGGACCAGCCGCCGGAGTGGCGGG |
| | ATGATATACCACCTTCCGTGATTCTTTATCTTCCATCTCACG |
| | GTAGATATCCACAGTGCAH |
| 188 (SEQ ID NO: 258) | VTGCCACACCATGCAGAGGACCAGCCGCCGGAGTGGCGGG |
| | ATGATATACAAACTTCCGTGATTCTTTATCTTCCATCTCACG |
| | GTAGATATCCACAGTGCAH |
| 189 (SEQ ID NO: 259) | VTGCCACACCATGCAGAGGACCAGCCGCCGGAGTGGCGGG |
| | ATGATATACGAACTTCCGTGATTCTTTATCTTCCATCTCACG |
| | GTAGATATCCACAGTGCAH |
| 190 (SEQ ID NO: 260) | VGGTAACAGCTTCAAAAAATGCATCTTCCCTTGGGCCAAAT |
| | GATCCAGCTCTGAACGTAATATCATTTGCAACAACAATAAT |
| | CTCCCTACCACTAGGAAAH |
| 191 (SEQ ID NO: 261) | VGGTAACAGCTTCAAAAAATGCATCTTCCCTTGGGCCAAAT |
| | GATCCAGCAATGAACGTAATATCATTTGCAACAACAATAAT |
| | CTCCCTACCACTAGGAAAH |
| 192 (SEQ ID NO: 262) | VGGTAACAGCTTCAAAAAATGCATCTTCCCTTGGGCCAAAT |
| | GATCCAGCGATGAACGTAATATCATTTGCAACAACAATAAT |
| | CTCCCTACCACTAGGAAAH |
| 193 (SEQ ID NO: 263) | VGGTAACAGCTTCAAAAAATGCATCTTCCCTTGGGCCAAAT |
| | GATCCAGCTATGAACGTAATATCATTTGCAACAACAATAAT |
| | CTCCCTACCACTAGGAAAH |
| 194 (SEQ ID NO: 264) | VGGTAACAGCTTCAAAAAATGCATCTTCCCTTGGGCCGAAA |
| | GAACCAGCAATGAACGTAATATCATTTGCAACAACAATAAT |
| | CTCCCTACCACTAGGAAAH |
| 195 (SEQ ID NO: 265) | VGGTAACAGCTTCAAAAAATGCATCTTCCCTTGGGCCGAAA |
| | GAACCAGCGATGAACGTAATATCATTTGCAACAACAATAAT |
| | CTCCCTACCACTAGGAAAH |
| 196 (SEQ ID NO: 266) | VGGTAACAGCTTCAAAAAATGCATCTTCCCTTGGGCCGAAA |
| | GAACCAGCTATGAACGTAATATCATTTGCAACAACAATAAT |
| | CTCCCTACCACTAGGAAAH |
| 197 (SEQ ID NO: 267) | VATTGCTCACGGGAATCAAGCTGACCAGGGTCAGCAGGGAT |
| | AGTTTGCATCATGGTCTGAGTCTCCACAGCTATCACACCCAC |
| | TGGAATTCCACCAAGCTTTH |
| 198 (SEQ ID NO: 268) | VATTGCTCACGGGAATCAAGCTGACCAGGGTCAGCAGGGAT |
| | AGTTTGCATAGTGGTCTGAGTCTCCACAGCTATCACACCCAC |
| | TGGAATTCCACCAAGCTTTH |
| 199 (SEQ ID NO: 269) | VATTGCTCACGGGAATCAAGCTGACCAGGGTCAGCAGGGAT |
| | AGTTTGCATGGTGGTCTGAGTCTCCACAGCTATCACACCCAC |
| | TGGAATTCCACCAAGCTTTH |
| 200 (SEQ ID NO: 270) | VATTGCTCACGGGAATCAAGCTGACCAGGGTCAGCAGGGAT |
| | AGTTTGCATTGTGGTCTGAGTCTCCACAGCTATCACACCCAC |
| | TGGAATTCCACCAAGCTTTH |
| 201 (SEQ ID NO: 271) | VATTGCTCACGGGAATCAAGCTGACCAGGGTCAGCAGGGAT |
| | AGTTTGCATCGTGGTCTGAGTCTCCACAGCTATCACACCCAC |
| | TGGAATTCCACCAAGCTTTH |
| 202 (SEQ ID NO: 272) | VATTGCTCACGGGAATCAAGCTGACCAGGGTCAGCAGGGAT |
| | AGTTTGCATAGTCGTCTGAGTCTCCACAGCTATCACACCCAC |
| | TGGAATTCCACCAAGCTTTH |
| 203 (SEQ ID NO: 273) | VATTGCTCACGGGAATCAAGCTGACCAGGGTCAGCAGGGAT |
| | AGTTTGCATGGTCGTCTGAGTCTCCACAGCTATCACACCCAC |
| | TGGAATTCCACCAAGCTTTH |
| 204 (SEQ ID NO: 274) | VATTGCTCACGGGAATCAAGCTGACCAGGGTCAGCAGGGAT |
| | AGTTTGCATTGTCGTCTGAGTCTCCACAGCTATCACACCCAC |
| | TGGAATTCCACCAAGCTTTH |
| 205 (SEQ ID NO: 275) | VATTGCTCACGGGAATCAAGCTGACCAGGGTCAGCAGGGAT |
| | AGTTTGCATCGTCGTCTGAGTCTCCACAGCTATCACACCCAC |
| | TGGAATTCCACCAAGCTTTH |
| 206 (SEQ ID NO: 276) | VATTGCTCACGGGAATCAAGCTGACCAGGGTCAGCAGGGAT |
| | AGTTTGCATAGTGGTCTGAGTCTCGACAGCTATCACACCCAC |
| | TGGAATTCCACCAAGCTTTH |
| 207 (SEQ ID NO: 277) | VATTGCTCACGGGAATCAAGCTGACCAGGGTCAGCAGGGAT |
| | AGTTTGCATGGTGGTCTGAGTCTCGACAGCTATCACACCCAC |
| | TGGAATTCCACCAAGCTTTH |
| 208 (SEQ ID NO: 278) | VATTGCTCACGGGAATCAAGCTGACCAGGGTCAGCAGGGAT |
| | AGTTTGCATTGTGGTCTGAGTCTCGACAGCTATCACACCCAC |
| | TGGAATTCCACCAAGCTTTH |
| 209 (SEQ ID NO: 279) | VATTGCTCACGGGAATCAAGCTGACCAGGGTCAGCAGGGAT |
| | AGTTTGCATCGTGGTCTGAGTCTCGACAGCTATCACACCCAC |
| | TGGAATTCCACCAAGCTTTH |
| 210 (SEQ ID NO: 280) | VGTGTAAATGTCTCCTTATATGCCCTAGAATAAGCACTGGCA |
| | ATAGCAGCACTTCCATGTATATTCTCCACACCAAGTCCATCT |
| | TCCTTGCCCACAACAGAAH |
| 211 (SEQ ID NO: 281) | VGTGTAAATGTCTCCTTATATGCCCTAGAATAAGCACTGGCA |
| | ATAGCAGCATTTCCATGTATATTCTCCACACCAAGTCCATCT |
| | TCCTTGCCCACAACAGAAH |
| 212 (SEQ ID NO: 282) | VGTGTAAATGTCTCCTTATATGCCCTAGAATAAGCACTGGCA |
| | ATAGCAGCGTTTCCATGTATATTCTCCACACCAAGTCCATCT |
| | TCCTTGCCCACAACAGAAH |
| 213 (SEQ ID NO: 283) | VGTGTAAATGTCTCCTTATATGCCCTAGAATAAGCACTGGCA |
| | ATAGCAGCATTTCCATGAATGTTCTCCACACCAAGTCCATCT |
| | TCCTTGCCCACAACAGAAH |
| 214 (SEQ ID NO: 284) | VGTGTAAATGTCTCCTTATATGCCCTAGAATAAGCACTGGCA |
| | ATAGCAGCGTTTCCATGAATGTTCTCCACACCAAGTCCATCT |
| | TCCTTGCCCACAACAGAAH |
| 215 (SEQ ID NO: 285) | VGTGTAAATGTCTCCTTATATGCCCTAGAATAAGCACTCGCA |
| | ATAGCAGCATTTCCATGTATATTCTCCACACCAAGTCCATCT |
| | TCCTTGCCCACAACAGAAH |
| 216 (SEQ ID NO: 286) | VGTGTAAATGTCTCCTTATATGCCCTAGAATAAGCACTCGCA |
| | ATAGCAGCGTTTCCATGTATATTCTCCACACCAAGTCCATCT |
| | TCCTTGCCCACAACAGAAH |
| 217 (SEQ ID NO: 287) | VAGCCTGTAAGAATAATAGGCTGGTCAAGACGCTGTATGCA |
| | CCGGATGCCAAGTCGAGCAAGATAAGCTCCTATTCCAACAG |
| | TTCTTCCAGTCACAAATGTAH |
| 218 (SEQ ID NO: 288) | VAGCCTGTAAGAATAATAGGCTGGTCAAGACGCTGTATGCA |
| | CCGGATGCCATGTCGAGCAAGATAAGCTCCTATTCCAACAG |
| | TTCTTCCAGTCACAAATGTAH |
| 219 (SEQ ID NO: 289) | VAGCCTGTAAGAATAATAGGCTGGTCAAGACGCTGTATGCA |
| | CCGGATGCCGTGTCGAGCAAGATAAGCTCCTATTCCAACAG |
| | TTCTTCCAGTCACAAATGTAH |
| 220 (SEQ ID NO: 290) | VAGCCTGTAAGAATAATAGGCTGGTCAAGACGCTGTATGCA |
| | CCGGATGCCATGACGAGCAAGATAAGCTCCTATTCCAACAG |
| | TTCTTCCAGTCACAAATGTAH |
| 221 (SEQ ID NO: 291) | VAGCCTGTAAGAATAATAGGCTGGTCAAGACGCTGTATGCA |
| | CCGGATGCCGTGACGAGCAAGATAAGCTCCTATTCCAACAG |
| | TTCTTCCAGTCACAAATGTAH |
| 222 (SEQ ID NO: 292) | VAGCCTGTAAGAATAATAGGCTGGTCAAGACGCTGTATGCA |
| | CCGTATGCCATGTCGAGCAAGATAAGCTCCTATTCCAACAG |
| | TTCTTCCAGTCACAAATGTAH |
| 223 (SEQ ID NO: 293) | VAGCCTGTAAGAATAATAGGCTGGTCAAGACGCTGTATGCA |
| | CCGTATGCCGTGTCGAGCAAGATAAGCTCCTATTCCAACAG |
| | TTCTTCCAGTCACAAATGTAH |
| 224 (SEQ ID NO: 294) | VAGCCTGTAAGAATAATAGGCTGGTCAAGACGCTGTATGCA |
| | GCGGATGCCATGTCGAGCAAGATAAGCTCCTATTCCAACAG |
| | TTCTTCCAGTCACAAATGTAH |
| 225 (SEQ ID NO: 295) | VAGCCTGTAAGAATAATAGGCTGGTCAAGACGCTGTATGCA |
| | GCGGATGCCGTGTCGAGCAAGATAAGCTCCTATTCCAACAG |
| | TTCTTCCAGTCACAAATGTAH |
| 226 (SEQ ID NO: 296) | VGAGGAAGTTTCTTCTCACAGGCTAGGTTGGTAACAGCTTC |
| | AAAAAATGCATCTTCCCTTGGGCCAAATGATCCAGCTCTGA |
| | ACGTAATATCATTTGCAACAH |
| 227 (SEQ ID NO: 297) | VGAGGAAGTTTCTTCTCACAGGCTAGGTTGGTAACAGCTTC |
| | AAAAAATGCTTCTTCCCTTGGGCCAAATGATCCAGCTCTGAA |
| | CGTAATATCATTTGCAACAH |
| 228 (SEQ ID NO: 298) | VGAGGAAGTTTCTTCTCACAGGCTAGGTTGGTAACAGCTTC |
| | AAAAAATGCCTCTTCCCTTGGGCCAAATGATCCAGCTCTGA |
| | ACGTAATATCATTTGCAACAH |
| 229 (SEQ ID NO: 299) | VGAGGAAGTTTCTTCTCACAGGCTAGGTTGGTAACAGCTTC |
| | AAAAAATGCTCCTTCCCTTGGGCCAAATGATCCAGCTCTGA |
| | ACGTAATATCATTTGCAACAH |
| 230 (SEQ ID NO: 300) | VGAGGAAGTTTCTTCTCACAGGCTAGGTTGGTAACAGCTTC |
| | AAAAAATGCACCTTCCCTTGGGCCAAATGATCCAGCTCTGA |
| | ACGTAATATCATTTGCAACAH |
| 231 (SEQ ID NO: 301) | VGAGGAAGTTTCTTCTCACAGGCTAGGTTGGTAACAGCTTC |
| | AAAAAATGCGCCTTCCCTTGGGCCAAATGATCCAGCTCTGA |
| | ACGTAATATCATTTGCAACAH |
| 232 (SEQ ID NO: 302) | VGAGGAAGTTTCTTCTCACAGGCTAGGTTGGTAACAGCTTC |
| | AAAAAATGCCCCTTCCCTTGGGCCAAATGATCCAGCTCTGA |
| | ACGTAATATCATTTGCAACAH |
| 233 (SEQ ID NO: 303) | VGAGGAAGTTTCTTCTCACAGGCTAGGTTGGTAACAGCTTC |
| | AAAAAATGCTTCTTCTCTTGGGCCAAATGATCCAGCTCTGAA |
| | CGTAATATCATTTGCAACAH |
| 234 (SEQ ID NO: 304) | VGAGGAAGTTTCTTCTCACAGGCTAGGTTGGTAACAGCTTC |
| | AAAAAATGCCTCTTCTCTTGGGCCAAATGATCCAGCTCTGAA |
| | CGTAATATCATTTGCAACAH |
| 235 (SEQ ID NO: 305) | VGAGGAAGTTTCTTCTCACAGGCTAGGTTGGTAACAGCTTC |
| | AAAAAATGCTCCTTCTCTTGGGCCAAATGATCCAGCTCTGAA |
| | CGTAATATCATTTGCAACAH |
| 236 (SEQ ID NO: 306) | VGAGGAAGTTTCTTCTCACAGGCTAGGTTGGTAACAGCTTC |
| | AAAAAATGCACCTTCTCTTGGGCCAAATGATCCAGCTCTGA |
| | ACGTAATATCATTTGCAACAH |
| 237 (SEQ ID NO: 307) | VGAGGAAGTTTCTTCTCACAGGCTAGGTTGGTAACAGCTTC |
| | AAAAAATGCGCCTTCTCTTGGGCCAAATGATCCAGCTCTGA |
| | ACGTAATATCATTTGCAACAH |
| 238 (SEQ ID NO: 308) | VGAGGAAGTTTCTTCTCACAGGCTAGGTTGGTAACAGCTTC |
| | AAAAAATGCCCCTTCTCTTGGGCCAAATGATCCAGCTCTGA |
| | ACGTAATATCATTTGCAACAH |
| 239 (SEQ ID NO: 309) | VGAGGAAGTTTCTTCTCACAGGCTAGGTTGGTAACAGCTTC |
| | AAAAAATGCTTCTTCCCTAGGGCCAAATGATCCAGCTCTGA |
| | ACGTAATATCATTTGCAACAH |
| 240 (SEQ ID NO: 310) | VGAGGAAGTTTCTTCTCACAGGCTAGGTTGGTAACAGCTTC |
| | AAAAAATGCCTCTTCCCTAGGGCCAAATGATCCAGCTCTGA |
| | ACGTAATATCATTTGCAACAH |
| 241 (SEQ ID NO: 311) | VGAGGAAGTTTCTTCTCACAGGCTAGGTTGGTAACAGCTTC |
| | AAAAAATGCTCCTTCCCTAGGGCCAAATGATCCAGCTCTGA |
| | ACGTAATATCATTTGCAACAH |
| 242 (SEQ ID NO: 312) | VGAGGAAGTTTCTTCTCACAGGCTAGGTTGGTAACAGCTTC |
| | AAAAAATGCACCTTCCCTAGGGCCAAATGATCCAGCTCTGA |
| | ACGTAATATCATTTGCAACAH |
| 243 (SEQ ID NO: 313) | VGAGGAAGTTTCTTCTCACAGGCTAGGTTGGTAACAGCTTC |
| | AAAAAATGCGCCTTCCCTAGGGCCAAATGATCCAGCTCTGA |
| | ACGTAATATCATTTGCAACAH |
| 244 (SEQ ID NO: 314) | VGAGGAAGTTTCTTCTCACAGGCTAGGTTGGTAACAGCTTC |
| | AAAAAATGCCCCTTCCCTAGGGCCAAATGATCCAGCTCTGA |
| | ACGTAATATCATTTGCAACAH |

Following PEG treatment, protoplasts in liquid media with or without agarose embedding were cultured and incubated in a rotatory shaker in the dark at 28° C. An exemplary range for embedding protoplasts in agarose includes, but is not limited to 0.625×10⁶ to 2.5×10⁶ cells/ml. Microcalli were released onto solid media with or without clethodim (exemplary concentration of 0.25-0.5 μM) or sethoxydim (exemplary concentration of 2.5 M) selection. Isolated callus containing the intended target mutation were identified using PCR and DNA sequencing methods known in the art and rice plants were regenerated from converted callus. Clones were generated having the above exemplary mutations using methods consistent with the above Examples and the clones were tested for clethodim tolerance.

### Example 5

Mutations at position N2097S and V2098A were combined to determine if point mutations at both positions gave increased resistance compared to either single point mutation. Each of these respective mutations (N2097S, V2098A and N2097S/V2098A) were generated by standard methods and introduced into a Saccharomyces cerevisiae pseudodiploid acc1Δ strain with a plasmid containing wild-type O. sativa ACCase gene. Once each of these mutations were introduced by recombination mediated mutagenesis, the OsACCase wild-type plasmid was removed, and mutation strains were confirmed by sequencing. The three strains, along with a series of controls including a WT strain (negative control), an I1781L mutant strain, and a D2078G mutant strain (positive control), were streaked onto plates with increasing concentrations of clethodim. To ensure that growth on these plates reflected herbicide tolerance, the galactose concentration was kept at 0.01% so that ACCase expression would be limiting. As expected, the V2098A change, as well as control D2078G and I1781L mutations, did produce tolerance when compared to the wild-type (wt) control strain. Results indicated that N2097S alone does not produce detectable clethodim tolerance; however, when N2097S was combined with V2098A the tolerance of the double mutant strain was notably increased compared to the V2098A change alone. These observations argue that N2097S is an enhancer of V2098A tolerance.

### Example 6

The I1781L/A1785G double mutation was introduced into a native rice Oryza sativa ACCase gene as follows. Rice protoplasts were isolated from cell suspension cultures initiated from mature seed-derived embryogenic calli. A GRON encoding the I1781L/A1785G double mutation and a transiently expressed CRISPR plasmid (pTD1556, encoding both Streptococcus pyogenes Cas9 and sgRNA) were introduced into rice protoplasts using PEG DNA delivery method. Specific GRON and sgRNA sequences are shown in the below table. Following PEG treatment, protoplasts were cultured in liquid medium and kept stationary in the dark at 26° C. for 2 weeks. After 2 weeks, the medium was refreshed and transferred on a rotatory shaker in the dark at 26° C. After 4-6 weeks, microcalli were plated onto selection medium containing the clethodim ACCase inhibitor. Isolated calli were sampled and then screened for the presence of the I1781L/A1785G edits. A callus scored as positive for the I1781L/A1785G edits using both an allele specific PCR assay and Sanger DNA sequencing. Shoots were then regenerated from the identified callus and the I1781L/A1785G edits was again sequence confirmed. The I1781L/A1785G GRON and sgRNA target sequence is listed below in Table 7.

TABLE 7

| | |
|---|---|
| sgRNA (SEQ ID NO: 41) | guugguguggagaauauaca |
| GRON (SEQ ID NO: 315) | VGGGTTATTGATTCTGTTGTGGGCAAGGAAGATGGAC |
| | TTGGTGTGGAGAATCTACATGGAAGTGGTGCTATTGC |
| | CAGTGCTTATTCTAGGGCATATAAGGAGACATTTACA |
| | CTTAH |

*All sequences 5′ to 3′; V = CY3; H = 3′DMT dC CPG

### Example 7

Edits at position S1784N and A1785G were combined to determine if point mutations at both of these two positions gave increased resistance compared to either single point mutation. Each of these respective mutations (S1784N, A1785G and S1784N/A1785G) were generated by standard methods and introduced into a Saccharomyces cerevisiae pseudodiploid acc1Δ strain with a plasmid containing wild-type O. sativa ACCase gene. Once each of these mutations were introduced by recombination mediated mutagenesis, the OsACCase wild-type plasmid was removed, and mutation strains were confirmed by sequencing. The three strains, along with a series of controls including a WT strain (negative control), an I1781L mutant strain, a D2078G mutant strain (positive control), and a V2075L mutant strain were streaked onto plates with increasing concentrations of clethodim. To ensure that growth on these plates reflected herbicide tolerance, the galactose concentration was kept at 0.01% so that ACCase expression would be limiting.

The results from these assays are listed below in Table 8. All strains grew in the absence of clethodim, indicating that expression of each of these mutant ACCase genes was not pleiotropic in terms of fitness. No obvious differences in growth were observed after 4 days of growth between the low galactose and high galactose plates, other than the faster rate of initial growth for the wt strain with low galactose. The wt strain failed to grow on all concentrations of clethodim tested, indicating that it was sensitive to this herbicide. The S1784N mutation grew on concentrations up to 0.25 mM, while the S1784N/A1785G double mutation grew on up to 1 mM, indicating that the combination of these two mutations was superior in terms of resistance to either individual mutation. The S1784N/A1785G mutation grew to a similar extent as the V2075L mutation, and the D2078G positive control grew on medium with up to 1.0 mM clethodim. The I1781L mutation, which has been shown to have only moderate resistance to clethodim, grew on concentrations up to 0.25 mM clethodim, consistent with moderate resistance. The chart below indicates the relative growth phenotypes of these strains. All strains were sequenced confirmed after plating results were obtained to ensure that their phenotypes matched their genotypes.

TABLE 8

| | | | | | | S1784N/ | | | |
|---|---|---|---|---|---|---|---|---|---|
| | | D2078G | I1781L | S1784N | A1785G | A1785G | V2075L | water | wt |
| highGal | | +++ | +++ | +++ | +++ | +++ | +++ | − | +++ |
| lowGal | | +++ | +++ | +++ | +++ | +++ | +++ | − | +++ |
| 0.05 | mM | +++ | +++ | +++ | (−) | +++ | +++ | − | (−) |
| 0.1 | mM | +++ | +++ | +++ | (−) | +++ | +++ | − | (−) |
| 0.25 | mM | +++ | ++ | + | − | +++ | +++ | − | (−) |
| 0.5 | mM | +++ | (−) | (−) | − | +++ | +++ | − | (−) |
| 1 | mM | + | (−) | (−) | − | + | + | − | (−) |
| 2 | mM | (−) | (−) | (−) | − | (−) | (−) | − | (−) |

+++ high growth
++ moderate growth
+ low to negligible growth
(−) some cell debris, no growth
− no growth or cell debris

### Example 8

The D2078G mutation was introduced into a native rice Oryza sativa ACCase gene utilizing PEG delivery of CRISPR-Cas and GRONs into protoplasts. Examples of target spacer sequences for sgRNA targeting the rice ACCase gene are shown, but not limited to, those listed in the below Table 9. Sequence information for exemplary GRONs is shown, but not limited to those listed in the below Table 10. Rice protoplasts were isolated from cell suspension cultures initiated from Cypress mature seed-derived embryogenic calli. A non-coding D2078G GRON with RNP (CRISPR-Cas9 protein plus gRNA complex) was introduced into rice protoplasts using PEG-mediated delivery method. Following PEG treatment, protoplasts were cultured in liquid medium and kept stationary in the dark at 26±2° C. for 2 weeks. After 2 weeks, culture plates were gently scraped to release the divided protoplasts from the bottom of the plates and added equal volume of fresh liquid medium. These cultures were transferred on a rotatory shaker and incubated in the dark at 26±2° C. Further, these cultures were refreshed every week. After 4-5 weeks, microcalli were plated on solid medium. Individual calli were sampled and sent for qPCR and next generation sequencing (NGS) to screen for the presence of the D2078G mutation. NGS identified the D2078G edited callus line which was then multiplied and transferred to the shoot regeneration medium. Regenerated shoots from D2078G edited callus line were sampled and screened to reconfirm the D2078G edit using both qPCR and NGS. In one shoot, qPCR and NGS confirmed the D2078G edit with an additional SNP mutation 13 bp downstream resulting in a P2083T mutation.

TABLE 9
Exemplary target spacer sequences for rice
ACCase sgRNA

| Sequence No. | 5′ to 3′ sequence |
|---|---|
| 1 (SEQ ID NO: 316) | gcattgagtgctatgctgag |
| 2 (SEQ ID NO: 317) | tctcagcatagcactcaatg |
| 3 (SEQ ID NO: 318) | gcatagcactcaatgcggtc |
| 4 (SEQ ID NO: 319) | catagcactcaatgcggtct |
| 5 (SEQ ID NO: 320) | ggaggggcttgggttgtcgt |
| 6 (SEQ ID NO: 321) | agagctacgaggaggggctt |
| 7 (SEQ ID NO: 322) | cagagctacgaggaggggct |
| 8 (SEQ ID NO: 323) | ggctgcagagctacgaggag |
| 9 (SEQ ID NO: 324) | tggctgcagagctacgagga |
| 10 (SEQ ID NO: 325) | atggctgcagagctacgagg |
| 11 (SEQ ID NO: 326) | tcctcgtagctctgcagcca |
| 12 (SEQ ID NO: 327) | cccatggctgcagagctacg |
| 13 (SEQ ID NO: 328) | cctcgtagctctgcagccat |
| 14 (SEQ ID NO: 329) | cagtcctctcagcatagcactcaa |
| 15 (SEQ ID NO: 330) | tcttgcttccaacgacaacccaag |
| 16 (SEQ ID NO: 331) | tcttgcttccaacgacaacccaag |
| 17 (SEQ ID NO: 332) | ggagctacgaggaggggctt |
| 18 (SEQ ID NO: 333) | aagcccctcctogtagctcc |
| 19 (SEQ ID NO: 334) | cttgggttgtcgttggaagcaaga |
| 20 (SEQ ID NO: 335) | cttgggttgtcgttggaagcaaga |
| 21 (SEQ ID NO: 336) | ttgagtgctatgctgagaggactg |
| 022 (SEQ ID NO: 337) | atggctgcagagctacgagg |
| 23 (SEQ ID NO: 338) | cgtagctctgcagccatggg |
| 24 (SEQ ID NO: 339) | tggctgcagagctacgagga |
| 25 (SEQ ID NO: 340) | cctcgtagctctgcagccat |
| 26 (SEQ ID NO: 341) | tcctcgtagctctgcagcca |
| 27 (SEQ ID NO: 342) | ctcctcgtagctctgcagcc |
| 28 (SEQ ID NO: 343) | agcccctcctcgtagctctg |
| 29 (SEQ ID NO: 344) | aagcccctcctcgtagctct |
| 30 (SEQ ID NO: 345) | acgacaacccaagcccctcc |
| 31 (SEQ ID NO: 346) | agaccgcattgagtgctatg |
| 32 (SEQ ID NO: 347) | gaccgcattgagtgctatgc |
| 33 (SEQ ID NO: 348) | cattgagtgctatgctgaga |
| 34 (SEQ ID NO: 349) | ctcagcatagcactcaatgc |

TABLE 10
Exemplary rice ACCase GRON Sequences

| Sequence No. | 5′ to 3′ sequence (V = CY3; H = 3′DMT dC CPG) |
|---|---|
| 1 (SEQ ID NO: 350) | TACATTCCCATGGCTGCAGAGCTACGAGGAGGGGCTTGGG |
| | TTGTCGTTGGAAGCAAGATAAACCCAGACCGCATTGAGTG |
| | CTATGCTGAGAGGACTGCAAA |
| 2 (SEQ ID NO: 351) | TACATTCCCATGGCTGCAGAGCTACGAGGAGGGGCTTGGG |
| | TTGTCGTTGGTAGCAAGATAAACCCAGACCGCATTGAGTGC |
| | TATGCTGAGAGGACTGCAAA |
| 3 (SEQ ID NO: 352) | TACATTCCCATGGCTGCAGAGCTACGAGGAGGGGCTTGGG |
| | TTGTCGTTGGCAGCAAGATAAACCCAGACCGCATTGAGTGC |
| | TATGCTGAGAGGACTGCAAA |
| 4 (SEQ ID NO: 353) | TACATTCCCATGGCTGCAGAGCTACGAGGAGGGGCTTGGG |
| | TTGTCGTTGGGAGCAAGATAAACCCAGACCGCATTGAGTG |
| | CTATGCTGAGAGGACTGCAAA |
| 5 (SEQ ID NO: 354) | TACATTCCCATGGCTGCAGAGCTACGAGGAGGGGCTTGGG |
| | TTGTCGTTGGAAGCAAGATAAACACAGACCGCATTGAGTG |
| | CTATGCTGAGAGGACTGCAAA |
| 6 (SEQ ID NO: 355) | TACATTCCCATGGCTGCAGAGCTACGAGGAGGGGCTTGGG |
| | TTGTCGTTGGTAGCAAGATAAACACAGACCGCATTGAGTGC |
| | TATGCTGAGAGGACTGCAAA |
| 7 (SEQ ID NO: 356) | TACATTCCCATGGCTGCAGAGCTACGAGGAGGGGCTTGGG |
| | TTGTCGTTGGCAGCAAGATAAACACAGACCGCATTGAGTG |
| | CTATGCTGAGAGGACTGCAAA |
| 8 (SEQ ID NO: 357) | TACATTCCCATGGCTGCAGAGCTACGAGGAGGGGCTTGGG |
| | TTGTCGTTGGGAGCAAGATAAACACAGACCGCATTGAGTG |
| | CTATGCTGAGAGGACTGCAAA |
| 9 (SEQ ID NO: 358) | TACATTCCCATGGCTGCAGAGCTACGAGGAGGGGCTTGGG |
| | TTGTCGTTGGAAGCAAGATAAACACGGACCGCATTGAGTG |
| | CTATGCTGAGAGGACTGCAAA |
| 10 (SEQ ID NO: 359) | TACATTCCCATGGCTGCAGAGCTACGAGGAGGGGCTTGGG |
| | TTGTCGTTGGTAGCAAGATAAACACGGACCGCATTGAGTGC |
| | TATGCTGAGAGGACTGCAAA |
| 11 (SEQ ID NO: 360) | TACATTCCCATGGCTGCAGAGCTACGAGGAGGGGCTTGGG |
| | TTGTCGTTGGCAGCAAGATAAACACGGACCGCATTGAGTG |
| | CTATGCTGAGAGGACTGCAAA |
| 12 (SEQ ID NO: 361) | TACATTCCCATGGCTGCAGAGCTACGAGGAGGGGCTTGGG |
| | TTGTCGTTGGGAGCAAGATAAACACGGACCGCATTGAGTG |
| | CTATGCTGAGAGGACTGCAAA |
| 13 (SEQ ID NO: 362) | TACATTCCCATGGCTGCAGAGCTACGAGGAGGGGCTTGGG |
| | TTGTCGTTGGAAGCAAGATAAACACTGACCGCATTGAGTGC |
| | TATGCTGAGAGGACTGCAAA |
| 14 (SEQ ID NO: 363) | TACATTCCCATGGCTGCAGAGCTACGAGGAGGGGCTTGGG |
| | TTGTCGTTGGTAGCAAGATAAACACTGACCGCATTGAGTGC |
| | TATGCTGAGAGGACTGCAAA |
| 15 (SEQ ID NO: 364) | TACATTCCCATGGCTGCAGAGCTACGAGGAGGGGCTTGGG |
| | TTGTCGTTGGCAGCAAGATAAACACTGACCGCATTGAGTGC |
| | TATGCTGAGAGGACTGCAAA |
| 16 (SEQ ID NO: 365) | TACATTCCCATGGCTGCAGAGCTACGAGGAGGGGCTTGGG |
| | TTGTCGTTGGGAGCAAGATAAACACTGACCGCATTGAGTGC |
| | TATGCTGAGAGGACTGCAAA |
| 17 (SEQ ID NO: 366) | TACATTCCCATGGCTGCAGAGCTACGAGGAGGGGCTTGGG |
| | TTGTCGTTGGAAGCAAGATAAACACCGACCGCATTGAGTG |
| | CTATGCTGAGAGGACTGCAAA |
| 18 (SEQ ID NO: 367) | TACATTCCCATGGCTGCAGAGCTACGAGGAGGGGCTTGGG |
| | TTGTCGTTGGTAGCAAGATAAACACCGACCGCATTGAGTGC |
| | TATGCTGAGAGGACTGCAAA |
| 19 (SEQ ID NO: 368) | TACATTCCCATGGCTGCAGAGCTACGAGGAGGGGCTTGGG |
| | TTGTCGTTGGCAGCAAGATAAACACCGACCGCATTGAGTGC |
| | TATGCTGAGAGGACTGCAAA |
| 20 (SEQ ID NO: 369) | TACATTCCCATGGCTGCAGAGCTACGAGGAGGGGCTTGGG |
| | TTGTCGTTGGGAGCAAGATAAACACCGACCGCATTGAGTG |
| | CTATGCTGAGAGGACTGCAAA |
| 21 (SEQ ID NO: 370) | TACATTCCCATGGCTGCAGAGCTACGAGGAGGGGCTTGGG |
| | TTGTCGTTGATAGCAAGATAAACACAGACCGCATTGAGTGC |
| | TATGCTGAGAGGACTGCAAA |
| 22 (SEQ ID NO: 371) | TACATTCCCATGGCTGCAGAGCTACGAGGAGGGGCTTGGG |
| | TTGTCGTTGATAGCAAGATAAACACGGACCGCATTGAGTGC |
| | TATGCTGAGAGGACTGCAAA |
| 23 (SEQ ID NO: 372) | TACATTCCCATGGCTGCAGAGCTACGAGGAGGGGCTTGGG |
| | TTGTCGTTGATAGCAAGATAAACACTGACCGCATTGAGTGC |
| | TATGCTGAGAGGACTGCAAA |
| 24 (SEQ ID NO: 349) | TACATTCCCATGGCTGCAGAGCTACGAGGAGGGGCTTGGG |
| | TTGTCGTTGATAGCAAGATAAACACCGACCGCATTGAGTGC |
| | TATGCTGAGAGGACTGCAAA |
| 25 (SEQ ID NO: 373) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGAAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 26 (SEQ ID NO: 374) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGTAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 27 (SEQ ID NO: 375) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGCAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 28 (SEQ ID NO: 376) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGGAGCAAGATAAACCCAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 29 (SEQ ID NO: 377) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGAAGCAAGATAAACACAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 30 (SEQ ID NO: 378) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGTAGCAAGATAAACACAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 31 (SEQ ID NO: 379) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGCAGCAAGATAAACACAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 32 (SEQ ID NO: 380) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGGAGCAAGATAAACACAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 33 (SEQ ID NO: 381) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGAAGCAAGATAAACACGGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 34 (SEQ ID NO: 382) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGTAGCAAGATAAACACGGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 35 (SEQ ID NO: 383) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGCAGCAAGATAAACACGGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 36 (SEQ ID NO: 384) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGGAGCAAGATAAACACGGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 37 (SEQ ID NO: 385) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGAAGCAAGATAAACACTGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 38 (SEQ ID NO: 386) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGTAGCAAGATAAACACTGACCGCA |
| | TTGAGTGCTATGCTGAGAGGACTGCAAAAGGCAATGTTCTG |
| | GAACCGCAAGGGTTAATTGAGATCAAGTTCAGGTCAG |
| 39 (SEQ ID NO: 387) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGCAGCAAGATAAACACTGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 40 (SEQ ID NO: 388) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGGAGCAAGATAAACACTGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 41(SEQ ID NO: 349) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGAAGCAAGATAAACACCGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 42 (SEQ ID NO: 389) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGTAGCAAGATAAACACCGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 43 (SEQ ID NO: 390) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGCAGCAAGATAAACACCGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 44 (SEQ ID NO: 391) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGGGAGCAAGATAAACACCGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 45 (SEQ ID NO: 392) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGATAGCAAGATAAACACAGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 46 (SEQ ID NO: 393) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGATAGCAAGATAAACACGGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 47 (SEQ ID NO: 394) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGATAGCAAGATAAACACTGACCGCA |
| | TTGAGTGCTATGCTGAGAGGACTGCAAAAGGCAATGTTCTG |
| | GAACCGCAAGGGTTAATTGAGATCAAGTTCAGGTCAG |
| 48 (SEQ ID NO: 395) | GCTCGACTATTGTTGAGAACCTTAGGACATACAATCAGCCT |
| | GCCTTTGTCTACATTCCCATGGCTGCAGAGCTACGAGGAGG |
| | GGCTTGGGTTGTCGTTGATAGCAAGATAAACACCGACCGC |
| | ATTGAGTGCTATGCTGAGAGGACTGCAAAAGGCAATGTTCT |
| | GGAACCGCAAGGGTTAATTGAGATCAAGTTCAGGTCAG |
| 49 (SEQ ID NO: 396) | TTTGCAGTCCTCTCAGCATAGCACTCAATGCGGTCTGGGTT |
| | TATCTTGCTTCCAACGACAACCCAAGCCCCTCCTCGTAGCT |
| | CTGCAGCCATGGGAATGTA |
| 50 (SEQ ID NO: 397) | TTTGCAGTCCTCTCAGCATAGCACTCAATGCGGTCTGGGTT |
| | TATCTTGCTACCAACGACAACCCAAGCCCCTCCTCGTAGCT |
| | CTGCAGCCATGGGAATGTA |
| 51 (SEQ ID NO: 398) | TTTGCAGTCCTCTCAGCATAGCACTCAATGCGGTCTGGGTT |
| | TATCTTGCTGCCAACGACAACCCAAGCCCCTCCTCGTAGCT |
| | CTGCAGCCATGGGAATGTA |
| 52 (SEQ ID NO: 399) | TTTGCAGTCCTCTCAGCATAGCACTCAATGCGGTCTGGGTT |
| | TATCTTGCTCCCAACGACAACCCAAGCCCCTCCTCGTAGCT |
| | CTGCAGCCATGGGAATGTA |
| 53 (SEQ ID NO: 400) | TTTGCAGTCCTCTCAGCATAGCACTCAATGCGGTCTGTGTTT |
| | ATCTTGCTTCCAACGACAACCCAAGCCCCTCCTCGTAGCTC |
| | TGCAGCCATGGGAATGTA |
| 54 (SEQ ID NO: 401) | TTTGCAGTCCTCTCAGCATAGCACTCAATGCGGTCTGTGTTT |
| | ATCTTGCTACCAACGACAACCCAAGCCCCTCCTCGTAGCTC |
| | TGCAGCCATGGGAATGTA |
| 55 (SEQ ID NO: 402) | TTTGCAGTCCTCTCAGCATAGCACTCAATGCGGTCTGTGTTT |
| | ATCTTGCTGCCAACGACAACCCAAGCCCCTCCTCGTAGCTC |
| | TGCAGCCATGGGAATGTA |
| 56 (SEQ ID NO: 403) | TTTGCAGTCCTCTCAGCATAGCACTCAATGCGGTCTGTGTTT |
| | ATCTTGCTCCCAACGACAACCCAAGCCCCTCCTCGTAGCTC |
| | TGCAGCCATGGGAATGTA |
| 57 (SEQ ID NO: 404) | TTTGCAGTCCTCTCAGCATAGCACTCAATGCGGTCCGTGTT |
| | TATCTTGCTTCCAACGACAACCCAAGCCCCTCCTCGTAGCT |
| | CTGCAGCCATGGGAATGTA |
| 58 (SEQ ID NO: 405) | TTTGCAGTCCTCTCAGCATAGCACTCAATGCGGTCCGTGTT |
| | TATCTTGCTACCAACGACAACCCAAGCCCCTCCTCGTAGCT |
| | CTGCAGCCATGGGAATGTA |
| 59 (SEQ ID NO: 406) | TTTGCAGTCCTCTCAGCATAGCACTCAATGCGGTCCGTGTT |
| | TATCTTGCTGCCAACGACAACCCAAGCCCCTCCTCGTAGCT |
| | CTGCAGCCATGGGAATGTA |
| 60 (SEQ ID NO: 407) | TTTGCAGTCCTCTCAGCATAGCACTCAATGCGGTCCGTGTT |
| | TATCTTGCTCCCAACGACAACCCAAGCCCCTCCTCGTAGCT |
| | CTGCAGCCATGGGAATGTA |
| 61 (SEQ ID NO: 408) | TTTGCAGTCCTCTCAGCATAGCACTCAATGCGGTCAGTGTT |
| | TATCTTGCTTCCAACGACAACCCAAGCCCCTCCTCGTAGCT |
| | CTGCAGCCATGGGAATGTA |
| 62 (SEQ ID NO: 409) | TTTGCAGTCCTCTCAGCATAGCACTCAATGCGGTCAGTGTT |
| | TATCTTGCTACCAACGACAACCCAAGCCCCTCCTCGTAGCT |
| | CTGCAGCCATGGGAATGTA |
| 63 (SEQ ID NO: 410) | TTTGCAGTCCTCTCAGCATAGCACTCAATGCGGTCAGTGTT |
| | TATCTTGCTGCCAACGACAACCCAAGCCCCTCCTCGTAGCT |
| | CTGCAGCCATGGGAATGTA |
| 64 (SEQ ID NO: 411) | TTTGCAGTCCTCTCAGCATAGCACTCAATGCGGTCAGTGTT |
| | TATCTTGCTCCCAACGACAACCCAAGCCCCTCCTCGTAGCT |
| | CTGCAGCCATGGGAATGTA |
| 65 (SEQ ID NO: 412) | TTTGCAGTCCTCTCAGCATAGCACTCAATGCGGTCGGTGTT |
| | TATCTTGCTTCCAACGACAACCCAAGCCCCTCCTCGTAGCT |
| | CTGCAGCCATGGGAATGTA |
| 66 (SEQ ID NO: 413) | TTTGCAGTCCTCTCAGCATAGCACTCAATGCGGTCGGTGTT |
| | TATCTTGCTACCAACGACAACCCAAGCCCCTCCTCGTAGCT |
| | CTGCAGCCATGGGAATGTA |
| 67 (SEQ ID NO: 414) | TTTGCAGTCCTCTCAGCATAGCACTCAATGCGGTCGGTGTT |
| | TATCTTGCTGCCAACGACAACCCAAGCCCCTCCTCGTAGCT |
| | CTGCAGCCATGGGAATGTA |
| 68 (SEQ ID NO: 415) | TTTGCAGTCCTCTCAGCATAGCACTCAATGCGGTCGGTGTT |
| | TATCTTGCTCCCAACGACAACCCAAGCCCCTCCTCGTAGCT |
| | CTGCAGCCATGGGAATGTA |
| 69 (SEQ ID NO: 416) | TTTGCAGTCCTCTCAGCATAGCACTCAATGCGGTCTGTGTTT |
| | ATCTTGCTATCAACGACAACCCAAGCCCCTCCTCGTAGCTC |
| | TGCAGCCATGGGAATGTA |
| 70 (SEQ ID NO: 417) | TTTGCAGTCCTCTCAGCATAGCACTCAATGCGGTCCGTGTT |
| | TATCTTGCTATCAACGACAACCCAAGCCCCTCCTCGTAGCT |
| | CTGCAGCCATGGGAATGTA |
| 71 (SEQ ID NO: 418) | TTTGCAGTCCTCTCAGCATAGCACTCAATGCGGTCAGTGTT |
| | TATCTTGCTATCAACGACAACCCAAGCCCCTCCTCGTAGCT |
| | CTGCAGCCATGGGAATGTA |
| 72 (SEQ ID NO: 419) | TTTGCAGTCCTCTCAGCATAGCACTCAATGCGGTCGGTGTT |
| | TATCTTGCTATCAACGACAACCCAAGCCCCTCCTCGTAGCT |
| | CTGCAGCCATGGGAATGTA |
| 73 (SEQ ID NO: 420) | CTGACCTGAACTTGATCTCAATTAACCCTTGCGGTTCCAGA |
| | ACATTGCCTTTTGCAGTCCTCTCAGCATAGCACTCAATGCG |
| | GTCTGGGTTTATCTTGCTTCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 74 (SEQ ID NO: 421) | CTGACCTGAACTTGATCTCAATTAACCCTTGCGGTTCCAGA |
| | ACATTGCCTTTTGCAGTCCTCTCAGCATAGCACTCAATGCG |
| | GTCTGGGTTTATCTTGCTACCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 75 (SEQ ID NO: 422) | CTGACCTGAACTTGATCTCAATTAACCCTTGCGGTTCCAGA |
| | ACATTGCCTTTTGCAGTCCTCTCAGCATAGCACTCAATGCG |
| | GTCTGGGTTTATCTTGCTGCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 76 (SEQ ID NO: 423) | CTGACCTGAACTTGATCTCAATTAACCCTTGCGGTTCCAGA |
| | ACATTGCCTTTTGCAGTCCTCTCAGCATAGCACTCAATGCG |
| | GTCTGGGTTTATCTTGCTCCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 77 (SEQ ID NO: 424) | CTGACCTGAACTTGATCTCAATTAACCCTTGCGGTTCCAGA |
| | ACATTGCCTTTTGCAGTCCTCTCAGCATAGCACTCAATGCG |
| | GTCTGTGTTTATCTTGCTTCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 78 (SEQ ID NO: 425) | CTGACCTGAACTTGATCTCAATTAACCCTTGCGGTTCCAGA |
| | ACATTGCCTTTTGCAGTCCTCTCAGCATAGCACTCAATGCG |
| | GTCTGTGTTTATCTTGCTACCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 79 (SEQ ID NO: 426) | CTGACCTGAACTTGATCTCAATTAACCCTTGCGGTTCCAGA |
| | ACATTGCCTTTTGCAGTCCTCTCAGCATAGCACTCAATGCG |
| | GTCTGTGTTTATCTTGCTGCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 80 (SEQ ID NO: 427) | CTGACCTGAACTTGATCTCAATTAACCCTTGCGGTTCCAGA |
| | ACATTGCCTTTTGCAGTCCTCTCAGCATAGCACTCAATGCG |
| | GTCTGTGTTTATCTTGCTCCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 81 (SEQ ID NO: 428) | CTGACCTGAACTTGATCTCAATTAACCCTTGCGGTTCCAGA |
| | ACATTGCCTTTTGCAGTCCTCTCAGCATAGCACTCAATGCG |
| | GTCCGTGTTTATCTTGCTTCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 82 (SEQ ID NO: 429) | CTGACCTGAACTTGATCTCAATTAACCCTTGCGGTTCCAGA |
| | ACATTGCCTTTTGCAGTCCTCTCAGCATAGCACTCAATGCG |
| | GTCCGTGTTTATCTTGCTACCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 83 (SEQ ID NO: 430) | CTGACCTGAACTTGATCTCAATTAACCCTTGCGGTTCCAGA |
| | ACATTGCCTTTTGCAGTCCTCTCAGCATAGCACTCAATGCG |
| | GTCCGTGTTTATCTTGCTGCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 84 (SEQ ID NO: 431) | CTGACCTGAACTTGATCTCAATTAACCCTTGCGGTTCCAGA |
| | ACATTGCCTTTTGCAGTCCTCTCAGCATAGCACTCAATGCG |
| | GTCCGTGTTTATCTTGCTCCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 85 (SEQ ID NO: 432) | CTGACCTGAACTTGATCTCAATTAACCCTTGCGGTTCCAGA |
| | ACATTGCCTTTTGCAGTCCTCTCAGCATAGCACTCAATGCG |
| | GTCAGTGTTTATCTTGCTTCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 86 (SEQ ID NO: 433) | CTGACCTGAACTTGATCTCAATTAACCCTTGCGGTTCCAGA |
| | ACATTGCCTTTTGCAGTCCTCTCAGCATAGCACTCAATGCG |
| | GTCAGTGTTTATCTTGCTACCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 87 (SEQ ID NO: 434) | CTGACCTGAACTTGATCTCAATTAACCCTTGCGGTTCCAGA |
| | ACATTGCCTTTTGCAGTCCTCTCAGCATAGCACTCAATGCG |
| | GTCAGTGTTTATCTTGCTGCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 88 (SEQ ID NO: 435) | CTGACCTGAACTTGATCTCAATTAACCCTTGCGGTTCCAGA |
| | ACATTGCCTTTTGCAGTCCTCTCAGCATAGCACTCAATGCG |
| | GTCAGTGTTTATCTTGCTCCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 89 (SEQ ID NO: 436) | CTGACCTGAACTTGATCTCAATTAACCCTTGCGGTTCCAGA |
| | ACATTGCCTTTTGCAGTCCTCTCAGCATAGCACTCAATGCG |
| | GTCGGTGTTTATCTTGCTTCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 90 (SEQ ID NO: 437) | CTGACCTGAACTTGATCTCAATTAACCCTTGCGGTTCCAGA |
| | ACATTGCCTTTTGCAGTCCTCTCAGCATAGCACTCAATGCG |
| | GTCGGTGTTTATCTTGCTACCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 91 (SEQ ID NO: 438) | CTGACCTGAACTTGATCTCAATTAACCCTTGCGGTTCCAGA |
| | ACATTGCCTTTTGCAGTCCTCTCAGCATAGCACTCAATGCG |
| | GTCGGTGTTTATCTTGCTGCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 92 (SEQ ID NO: 439) | CTGACCTGAACTTGATCTCAATTAACCCTTGCGGTTCCAGA |
| | ACATTGCCTTTTGCAGTCCTCTCAGCATAGCACTCAATGCG |
| | GTCGGTGTTTATCTTGCTCCCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 93 (SEQ ID NO: 440) | CTGACCTGAACTTGATCTCAATTAACCCTTGCGGTTCCAGA |
| | ACATTGCCTTTTGCAGTCCTCTCAGCATAGCACTCAATGCG |
| | GTCTGTGTTTATCTTGCTATCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 94 (SEQ ID NO: 441) | CTGACCTGAACTTGATCTCAATTAACCCTTGCGGTTCCAGA |
| | ACATTGCCTTTTGCAGTCCTCTCAGCATAGCACTCAATGCG |
| | GTCCGTGTTTATCTTGCTATCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 95 (SEQ ID NO: 442) | CTGACCTGAACTTGATCTCAATTAACCCTTGCGGTTCCAGA |
| | ACATTGCCTTTTGCAGTCCTCTCAGCATAGCACTCAATGCG |
| | GTCAGTGTTTATCTTGCTATCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |
| 96 (SEQ ID NO: 443) | CTGACCTGAACTTGATCTCAATTAACCCTTGCGGTTCCAGA |
| | ACATTGCCTTTTGCAGTCCTCTCAGCATAGCACTCAATGCG |
| | GTCGGTGTTTATCTTGCTATCAACGACAACCCAAGCCCCTC |
| | CTCGTAGCTCTGCAGCCATGGGAATGTAGACAAAGGCAGG |
| | CTGATTGTATGTCCTAAGGTTCTCAACAATAGTCGAGC |

### Example 9

The regenerated D2078G edited shoots were established in soil for greenhouse seed production and subsequently used for greenhouse trait confirmation and field validation. The D2078G edited shoots showed normal plant growth and strong herbicide tolerance but sterility due to poor pollen viability and inability to produce seeds by self-pollination.

The D2078G/P2083T edited shoot exhibited normal growth rate, herbicide tolerance, and fertility. The results of herbicide rate tolerance field trials of the D2078G/P2083T line can be seen below in Table 11. The edited plants were sprayed with clethodim, and plant injury was visually scored at (7) seven, (14) fourteen, and (28) days after application of the herbicide (“DAA”) on a 0-100% percentage scale for chlorosis of newest leaf-tips and growing points. The edited plants showed increased yield, low plant injury, and limited meristem necrosis except at the highest rates of herbicide application compared to the untreated control plants.

TABLE 11

| | Phytotoxicity | Phytotoxicity | Phytotoxicity | Yield |
|---|---|---|---|---|
| | (0-100%) | (0-100%) | (0-100%) | (% of |
| | 7 DAA | 14 DAA | 28 DAA | UTC) |
| Untreated control (UTC) | 0 | 0 | 0 | 100 |
| Dakota 1.6 oz/ac | 10 | 1.7 | 0 | 108.2546 |
| Dakota 2.5 oz/ac | 8.33 | 2.5 | 0 | 102.1424 |
| Dakota 3.3 oz/ac | 10 | 2.5 | 0 | 102.6465 |
| Dakota 4.9 oz/ac | 8.33 | 7.5 | 16.67 | 93.38374 |
| Dakota 6.5 oz/ac | 10 | 18.3 | 46.67 | 88.09074 |
| Dakota 1.6 oz/ac + 1.6 | 5 | 3.3 | 0 | 103.8437 |
| oz/ac | | | | |
| Dakota 2.5 oz/ac + 2.5 | 8.33 | 5.8 | 6.67 | 102.3314 |
| oz/ac | | | | |
| Dakota 3.3 oz/ac + 3.3 | 6.67 | 5 | 3.33 | 104.7259 |
| oz/ac | | | | |

25% = 25% crop response (chlorosis of newest leaf and growing point) compared to UTC
50% = 50% crop response (chlorosis of newest leaf and growing point) compared to UTC
75% = 75% crop response (chlorosis of newest leaf and growing point) compared to UTC
100% = 100% crop response (chlorosis of newest leaf and growing point) compared to UTC

### Example 10

Mutations of plasmids were made at positions P2083T and D2078G/P2083T in a yeast complementation assay and tested to determine if either gave comparable or increased resistance compared to D2078G alone. Each of these respective mutations (D2078G, P2083T, D2078G/P2083T) were generated by standard methods and introduced into a Saccharomyces cerevisiae pseudodiploid acc14 strain with a plasmid containing the wild-type O. sativa ACCase gene. Once plasmids bearing each of these mutations, introduced using recombination-mediated mutagenesis, the OsACCase wild-type plasmid was removed, and strains bearing each mutant plasmid were confirmed by sequencing. The three strains, along with a series of controls including a wild-type strain (negative control), an I1781L mutant strain (medium control), and a pre-existing D2078G mutant strain (positive control) were streaked onto plates with increasing concentrations of clethodim. To ensure that growth on these plates reflected herbicide tolerance, the galactose concentration was kept at 0.01% so that ACCase expression would be limiting.

The results from these assays can be seen below in Table 12. All strains grew in the absence of clethodim, indicating that expression of each of these mutant ACCase genes was not pleiotropic in to the strain's fitness. The wild-type strain failed to grow on all concentrations of clethodim tested, indicating that it was sensitive to this herbicide. The strain bearing the plasmid bearing the P2083T single mutation resembled the wild-type and was unable to grow on all concentrations of clethodim tested. The strain bearing the plasmid with the I1781L mutation, which has been shown to have only moderate resistance to clethodim, grew well on concentrations up to 0.1 mM clethodim and grew poorly on concentrations up to 0.25 mM clethodim, consistent with moderate resistance. Both the D2078G positive control and the D2078G/P2083T double mutant grew well on concentrations up to 0.5 mM clethodim and grew poorly on concentrations up to 1 mM clethodim, which was the highest concentration tested.

TABLE 12

| | 0 | 0.025 | 0.1 | 0.25 | 0.5 | 1 |
|---|---|---|---|---|---|---|
| | mM | mM | mM | mM | mM | mM |
| Wild-type | ++ | − | − | − | − | − |
| I1781L | ++ | ++ | ++ | + | − | − |
| D2078G | ++ | ++ | ++ | ++ | ++ | + |
| P2083T | ++ | − | − | − | − | − |
| D2078G/P2083T | ++ | ++ | ++ | ++ | ++ | + |

++ = Strong growth
+ = Poor growth
− = No growth

### Example 11

Field trials were conducted across the southern USA rice-growing regions to validate herbicide tolerance of the D2078G/P2083T edited rice line to clethodim. The clethodim herbicide chosen contained 2.0 pounds active ingredient per gallon. Using a randomized completed block design with three replicates, four clethodim spray rates (5.76, 8.30, 12.10 and 16.00 fluid ounces per acre) were compared to an untreated control (“UTC”). Phytotoxicity rates to access crop response were visually scored (7) seven, (14) fourteen and (28) twenty-eight days after clethodim application (“DAA”) using a 0-100% percentage chlorosis scale. All plots were maintained weed free using additional herbicides tolerant by all rice lines. Yield was collected using a research plot combine to harvest the small plot, and yields were standardized to 12.5% moisture before being converted to a percentage of the untreated control.

The results of the herbicide rate tolerance field trials of the D2078G/P2083T line can be seen below in Table 13. The edited rice lines exhibited normal growth, low plant injury and good yield across all spray rates in the nine environments tested.

TABLE 13

| | Phytotoxicity | Phytotoxicity | Phytotoxicity | Yield |
|---|---|---|---|---|
| | (0-100%) | (0-100%) | (0-100%) | (% of |
| | 7 DAA | 14 DAA | 28 DAA | UTC) |
| Untreated control (UTC) | 0.74 | 1.22 | 0.74 | 100.0 |
| Clethodim 2E - 5.76 fl oz/ac | 6.48 | 9.63 | 0.74 | 102.1 |
| Clethodim 2E - 8.30 fl oz/ac | 9.81 | 13.7 | 1.85 | 99.1 |
| Clethodim 2E - 12.10 fl oz/ac | 12.96 | 18.7 | 2.22 | 95.7 |
| Clethodim 2E - 16.00 fl oz/ac | 13.15 | 21.11 | 3.70 | 92.7 |

25% = 25% crop response (yellowing or chlorosis of newest leaf and growing point) compared to UTC
50% = 50% crop response (yellowing or chlorosis of newest leaf and growing point) compared to UTC
75% = 75% crop response (yellowing or chlorosis of newest leaf and growing point) compared to UTC
100% = 100% crop response (yellowing or chlorosis of newest leaf and growing point) compared to UTC

### Example 12

A method to introduce targeted mutations into a native wheat Triticum aestivum ACCase gene utilizes PEG delivery of CRISPR-Cas9/Cas12a and GRONs into protoplasts. Examples of target spacer sequences for sgRNA targeting the wheat ACCase gene are shown, but not limited to, those listed in the below Table 14. Sequence information for exemplary GRONs is shown, but not limited to those listed in the below Table 15.

TABLE 14
Exemplary target spacer
sequences for wheat ACCase sgRNA

| Sequence No. | 5′ to 3′ sequence |
|---|---|
| 1 (SEQ ID NO: 444) | ggttgatcgagatcaagttc |
| 2 (SEQ ID NO: 445) | ttgatctcgatcaacccttg |
| 3 (SEQ ID NO: 446) | gcaatgttctcgaacctcaa |
| 4 (SEQ ID NO: 447) | ggcaatgttctcgaacctca |
| 5 (SEQ ID NO: 448) | tatgctgagaggactgcaaa |
| 6 (SEQ ID NO: 449) | ctatgctgagaggactgcaa |
| 7 (SEQ ID NO: 450) | gcattgagttctatgctgag |
| 8 (SEQ ID NO: 451) | gcatagaactcaatgcgatc |
| 9 (SEQ ID NO: 452) | agagctacgtggaggggctt |
| 10 (SEQ ID NO: 453) | cagagctacgtggaggggct |
| 11 (SEQ ID NO: 454) | ggctgcagagctacgtggag |
| 12 (SEQ ID NO: 455) | aggctgcagagctacgtgga |
| 13 (SEQ ID NO: 456) | aaggctgcagagctacgtgg |
| 14 (SEQ ID NO: 457) | tccacgtagctctgcagcct |
| 15 (SEQ ID NO: 458) | cccaaggctgcagagctacg |
| 16 (SEQ ID NO: 459) | ccacgtagctctgcagcctt |
| 17 (SEQ ID NO: 460) | cacgtagctctgcagccttg |
| 18 (SEQ ID NO: 461) | agccttggggatatatacaa |
| 19 (SEQ ID NO: 462) | tgcctttgtatatatcccca |
| 20 (SEQ ID NO: 463) | ttggggatatatacaaaggc |
| 21 (SEQ ID NO: 464) | caggctgattgtatgtccta |
| 22 (SEQ ID NO: 465) | caacaattgttgagaacctt |
| 23 (SEQ ID NO: 466) | cagtcctctcagcatagaactcaa |
| 24 (SEQ ID NO: 467) | tcttgctatcaatcacgacccaag |
| 25 (SEQ ID NO: 468) | tatatatccccaaggctgcagagc |
| 26 (SEQ ID NO: 469) | gaacttgatctcgatcaacc |
| 27 (SEQ ID NO: 470) | caagggttgatcgagatcaa |
| 28 (SEQ ID NO: 471) | ttgaggttcgagaacattgc |
| 29 (SEQ ID NO: 472) | tgaggttcgagaacattgcc |
| 30 (SEQ ID NO: 473) | tttgcagtcctctcagcata |
| 31 (SEQ ID NO: 474) | ttgcagtcctctcagcatag |
| 32 (SEQ ID NO: 475) | ctcagcatagaactcaatgc |
| 33 (SEQ ID NO: 476) | gatcgcattgagttctatgc |
| 34 (SEQ ID NO: 477) | aagcccctccacgtagctct |
| 35 (SEQ ID NO: 478) | agcccctccacgtagctctg |
| 36 (SEQ ID NO: 479) | ctccacgtagctctgcagcc |
| 37 (SEQ ID NO: 480) | tccacgtagctctgcagcct |
| 38 (SEQ ID NO: 481) | ccacgtagctctgcagcctt |
| 39 (SEQ ID NO: 482) | aggctgcagagctacgtgga |
| 40 (SEQ ID NO: 483) | cgtagctctgcagccttggg |
| 41 (SEQ ID NO: 484) | aaggctgcagagctacgtgg |
| 42 (SEQ ID NO: 485) | caaggctgcagagctacgtg |
| 43 (SEQ ID NO: 486) | ttgtatatatccccaaggct |
| 44 (SEQ ID NO: 487) | tggggatatatacaaaggca |
| 45 (SEQ ID NO: 488) | gcctttgtatatatccccaa |
| 46 (SEQ ID NO: 489) | taggacatacaatcagcctg |
| 47 (SEQ ID NO: 490) | aaggttctcaacaattgttg |
| 48 (SEQ ID NO: 491) | ttgagttctatgctgagaggactg |
| 49 (SEQ ID NO: 492) | cttgggtcgtgattgatagcaaga |
| 50 (SEQ ID NO: 493) | gctctgcagccttggggatatata |

TABLE 15
Exemplary wheat ACCase GRON Sequences

| Sequence No. | 5′ to 3′ (V = CY3; H = 3′DMT dC CPG) |
|---|---|
| 1 (SEQ ID NO: 494) | TATATCCCCAAGGCTGCAGAGCTACGTGGAGGGGCTTGGGTC |
| | GTGATTGGTAGCAAGATAAATCCAGATCGCATTGAGTTCTATG |
| | CTGAGAGGACTGCAAA |
| 2 (SEQ ID NO: 495) | TATATCCCCAAGGCTGCAGAGCTACGTGGAGGGGCTTGGGTC |
| | GTGATTGGCAGCAAGATAAATCCAGATCGCATTGAGTTCTAT |
| | GCTGAGAGGACTGCAAA |
| 3 (SEQ ID NO: 496) | TATATCCCCAAGGCTGCAGAGCTACGTGGAGGGGCTTGGGTC |
| | GTGATTGGAAGCAAGATAAATCCAGATCGCATTGAGTTCTAT |
| | GCTGAGAGGACTGCAAA |
| 4 (SEQ ID NO: 497) | TATATCCCCAAGGCTGCAGAGCTACGTGGAGGGGCTTGGGTC |
| | GTGATTGGGAGCAAGATAAATCCAGATCGCATTGAGTTCTAT |
| | GCTGAGAGGACTGCAAA |
| 5 (SEQ ID NO: 498) | TATATCCCCAAGGCTGCAGAGCTACGTGGAGGGGCTTGGGTC |
| | GTGATTGATAGCAAGATAAATACTGATCGCATTGAGTTCTATG |
| | CTGAGAGGACTGCAAA |
| 6 (SEQ ID NO: 499) | TATATCCCCAAGGCTGCAGAGCTACGTGGAGGGGCTTGGGTC |
| | GTGATTGATAGCAAGATAAATACCGATCGCATTGAGTTCTATG |
| | CTGAGAGGACTGCAAA |
| 7 (SEQ ID NO: 500) | TATATCCCCAAGGCTGCAGAGCTACGTGGAGGGGCTTGGGTC |
| | GTGATTGATAGCAAGATAAATACAGATCGCATTGAGTTCTAT |
| | GCTGAGAGGACTGCAAA |
| 8 (SEQ ID NO: 501) | TATATCCCCAAGGCTGCAGAGCTACGTGGAGGGGCTTGGGTC |
| | GTGATTGATAGCAAGATAAATACGGATCGCATTGAGTTCTAT |
| | GCTGAGAGGACTGCAAA |
| 9 (SEQ ID NO: 502) | TATATCCCCAAGGCTGCAGAGCTACGTGGAGGGGCTTGGGTC |
| | GTGATTGGTAGCAAGATAAATACTGATCGCATTGAGTTCTATG |
| | CTGAGAGGACTGCAAA |
| 10 (SEQ ID NO: 503) | TATATCCCCAAGGCTGCAGAGCTACGTGGAGGGGCTTGGGTC |
| | GTGATTGGCAGCAAGATAAATACTGATCGCATTGAGTTCTATG |
| | CTGAGAGGACTGCAAA |
| 11 (SEQ ID NO: 504) | TATATCCCCAAGGCTGCAGAGCTACGTGGAGGGGCTTGGGTC |
| | GTGATTGGAAGCAAGATAAATACTGATCGCATTGAGTTCTAT |
| | GCTGAGAGGACTGCAAA |
| 12 (SEQ ID NO: 505) | TATATCCCCAAGGCTGCAGAGCTACGTGGAGGGGCTTGGGTC |
| | GTGATTGGGAGCAAGATAAATACTGATCGCATTGAGTTCTAT |
| | GCTGAGAGGACTGCAAA |
| 13 (SEQ ID NO: 506) | TATATCCCCAAGGCTGCAGAGCTACGTGGAGGGGCTTGGGTC |
| | GTGATTGGTAGCAAGATAAATACCGATCGCATTGAGTTCTATG |
| | CTGAGAGGACTGCAAA |
| 14 (SEQ ID NO: 507) | TATATCCCCAAGGCTGCAGAGCTACGTGGAGGGGCTTGGGTC |
| | GTGATTGGCAGCAAGATAAATACCGATCGCATTGAGTTCTAT |
| | GCTGAGAGGACTGCAAA |
| 15 (SEQ ID NO: 508) | TATATCCCCAAGGCTGCAGAGCTACGTGGAGGGGCTTGGGTC |
| | GTGATTGGAAGCAAGATAAATACCGATCGCATTGAGTTCTAT |
| | GCTGAGAGGACTGCAAA |
| 16 (SEQ ID NO: 509) | TATATCCCCAAGGCTGCAGAGCTACGTGGAGGGGCTTGGGTC |
| | GTGATTGGGAGCAAGATAAATACCGATCGCATTGAGTTCTAT |
| | GCTGAGAGGACTGCAAA |
| 17 (SEQ ID NO: 510) | TATATCCCCAAGGCTGCAGAGCTACGTGGAGGGGCTTGGGTC |
| | GTGATTGGTAGCAAGATAAATACAGATCGCATTGAGTTCTAT |
| | GCTGAGAGGACTGCAAA |
| 18 (SEQ ID NO: 511) | TATATCCCCAAGGCTGCAGAGCTACGTGGAGGGGCTTGGGTC |
| | GTGATTGGCAGCAAGATAAATACAGATCGCATTGAGTTCTAT |
| | GCTGAGAGGACTGCAAA |
| 19 (SEQ ID NO: 512) | TATATCCCCAAGGCTGCAGAGCTACGTGGAGGGGCTTGGGTC |
| | GTGATTGGAAGCAAGATAAATACAGATCGCATTGAGTTCTAT |
| | GCTGAGAGGACTGCAAA |
| 20 (SEQ ID NO: 513) | TATATCCCCAAGGCTGCAGAGCTACGTGGAGGGGCTTGGGTC |
| | GTGATTGGGAGCAAGATAAATACAGATCGCATTGAGTTCTAT |
| | GCTGAGAGGACTGCAAA |
| 21 (SEQ ID NO: 514) | TATATCCCCAAGGCTGCAGAGCTACGTGGAGGGGCTTGGGTC |
| | GTGATTGGTAGCAAGATAAATACGGATCGCATTGAGTTCTAT |
| | GCTGAGAGGACTGCAAA |
| 22 (SEQ ID NO: 515) | TATATCCCCAAGGCTGCAGAGCTACGTGGAGGGGCTTGGGTC |
| | GTGATTGGCAGCAAGATAAATACGGATCGCATTGAGTTCTAT |
| | GCTGAGAGGACTGCAAA |
| 23 (SEQ ID NO: 516) | TATATCCCCAAGGCTGCAGAGCTACGTGGAGGGGCTTGGGTC |
| | GTGATTGGAAGCAAGATAAATACGGATCGCATTGAGTTCTAT |
| | GCTGAGAGGACTGCAAA |
| 24 (SEQ ID NO: 517) | TATATCCCCAAGGCTGCAGAGCTACGTGGAGGGGCTTGGGTC |
| | GTGATTGGGAGCAAGATAAATACGGATCGCATTGAGTTCTAT |
| | GCTGAGAGGACTGCAAA |
| 25 (SEQ ID NO: 518) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGTAGCAAGATAAATCCAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 26 (SEQ ID NO: 519) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGCAGCAAGATAAATCCAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 27 (SEQ ID NO: 520) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGAAGCAAGATAAATCCAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 28 (SEQ ID NO: 521) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGGAGCAAGATAAATCCAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 29 (SEQ ID NO: 522) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGATAGCAAGATAAATACTGATCGCATTGA |
| | GTTCTATGCTGAGAGGACTGCAAAGGGCAATGTTCTCGAACC |
| | TCAAGGGTTGATCGAGATCAAGTTCAGGTCAG |
| 30 (SEQ ID NO: 523) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGATAGCAAGATAAATACCGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 31 (SEQ ID NO: 524) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGATAGCAAGATAAATACAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 32 (SEQ ID NO: 525) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGATAGCAAGATAAATACGGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 33 (SEQ ID NO: 526) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGTAGCAAGATAAATACTGATCGCATTGA |
| | GTTCTATGCTGAGAGGACTGCAAAGGGCAATGTTCTCGAACC |
| | TCAAGGGTTGATCGAGATCAAGTTCAGGTCAG |
| 34 (SEQ ID NO: 527) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGCAGCAAGATAAATACTGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 35 (SEQ ID NO: 528) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGAAGCAAGATAAATACTGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 36 (SEQ ID NO: 529) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGGAGCAAGATAAATACTGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 37 (SEQ ID NO: 530) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGTAGCAAGATAAATACCGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 38 (SEQ ID NO: 531) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGCAGCAAGATAAATACCGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 39 (SEQ ID NO: 532) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGAAGCAAGATAAATACCGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 40 (SEQ ID NO: 533) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGGAGCAAGATAAATACCGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 41 (SEQ ID NO: 534) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGTAGCAAGATAAATACAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 42 (SEQ ID NO: 535) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGCAGCAAGATAAATACAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 43 (SEQ ID NO: 536) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGAAGCAAGATAAATACAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 44 (SEQ ID NO: 537) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGGAGCAAGATAAATACAGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 45 (SEQ ID NO: 538) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGTAGCAAGATAAATACGGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 46 (SEQ ID NO: 539) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGCAGCAAGATAAATACGGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 47 (SEQ ID NO: 540) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGAAGCAAGATAAATACGGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 48 (SEQ ID NO: 541) | GGTCAACAATTGTTGAGAACCTTAGGACATACAATCAGCCTG |
| | CCTTTGTATATATCCCCAAGGCTGCAGAGCTACGTGGAGGGG |
| | CTTGGGTCGTGATTGGGAGCAAGATAAATACGGATCGCATTG |
| | AGTTCTATGCTGAGAGGACTGCAAAGGGCAATGTTCTCGAAC |
| | CTCAAGGGTTGATCGAGATCAAGTTCAGGTCAG |
| 49 (SEQ ID NO: 542) | TTTGCAGTCCTCTCAGCATAGAACTCAATGCGATCTGGATTTA |
| | TCTTGCTACCAATCACGACCCAAGCCCCTCCACGTAGCTCTGC |
| | AGCCTTGGGGATATA |
| 50 (SEQ ID NO: 543) | TTTGCAGTCCTCTCAGCATAGAACTCAATGCGATCTGGATTTA |
| | TCTTGCTGCCAATCACGACCCAAGCCCCTCCACGTAGCTCTGC |
| | AGCCTTGGGGATATA |
| 51 (SEQ ID NO: 544) | TTTGCAGTCCTCTCAGCATAGAACTCAATGCGATCTGGATTTA |
| | TCTTGCTTCCAATCACGACCCAAGCCCCTCCACGTAGCTCTGC |
| | AGCCTTGGGGATATA |
| 52 (SEQ ID NO: 545) | TTTGCAGTCCTCTCAGCATAGAACTCAATGCGATCTGGATTTA |
| | TCTTGCTCCCAATCACGACCCAAGCCCCTCCACGTAGCTCTGC |
| | AGCCTTGGGGATATA |
| 53 (SEQ ID NO: 546) | TTTGCAGTCCTCTCAGCATAGAACTCAATGCGATCAGTATTTA |
| | TCTTGCTATCAATCACGACCCAAGCCCCTCCACGTAGCTCTGC |
| | AGCCTTGGGGATATA |
| 54 (SEQ ID NO: 547) | TTTGCAGTCCTCTCAGCATAGAACTCAATGCGATCGGTATTTA |
| | TCTTGCTATCAATCACGACCCAAGCCCCTCCACGTAGCTCTGC |
| | AGCCTTGGGGATATA |
| 55 (SEQ ID NO: 548) | TTTGCAGTCCTCTCAGCATAGAACTCAATGCGATCTGTATTTA |
| | TCTTGCTATCAATCACGACCCAAGCCCCTCCACGTAGCTCTGC |
| | AGCCTTGGGGATATA |
| 56 (SEQ ID NO: 549) | TTTGCAGTCCTCTCAGCATAGAACTCAATGCGATCCGTATTTA |
| | TCTTGCTATCAATCACGACCCAAGCCCCTCCACGTAGCTCTGC |
| | AGCCTTGGGGATATA |
| 57 (SEQ ID NO: 550) | TTTGCAGTCCTCTCAGCATAGAACTCAATGCGATCAGTATTTA |
| | TCTTGCTACCAATCACGACCCAAGCCCCTCCACGTAGCTCTGC |
| | AGCCTTGGGGATATA |
| 58 (SEQ ID NO: 551) | TTTGCAGTCCTCTCAGCATAGAACTCAATGCGATCAGTATTTA |
| | TCTTGCTGCCAATCACGACCCAAGCCCCTCCACGTAGCTCTGC |
| | AGCCTTGGGGATATA |
| 59 (SEQ ID NO: 552) | TTTGCAGTCCTCTCAGCATAGAACTCAATGCGATCAGTATTTA |
| | TCTTGCTTCCAATCACGACCCAAGCCCCTCCACGTAGCTCTGC |
| | AGCCTTGGGGATATA |
| 60 (SEQ ID NO: 553) | TTTGCAGTCCTCTCAGCATAGAACTCAATGCGATCAGTATTTA |
| | TCTTGCTCCCAATCACGACCCAAGCCCCTCCACGTAGCTCTGC |
| | AGCCTTGGGGATATA |
| 61 (SEQ ID NO: 554) | TTTGCAGTCCTCTCAGCATAGAACTCAATGCGATCGGTATTTA |
| | TCTTGCTACCAATCACGACCCAAGCCCCTCCACGTAGCTCTGC |
| | AGCCTTGGGGATATA |
| 62 (SEQ ID NO: 555) | TTTGCAGTCCTCTCAGCATAGAACTCAATGCGATCGGTATTTA |
| | TCTTGCTGCCAATCACGACCCAAGCCCCTCCACGTAGCTCTGC |
| | AGCCTTGGGGATATA |
| 63 (SEQ ID NO: 556) | TTTGCAGTCCTCTCAGCATAGAACTCAATGCGATCGGTATTTA |
| | TCTTGCTTCCAATCACGACCCAAGCCCCTCCACGTAGCTCTGC |
| | AGCCTTGGGGATATA |
| 64 (SEQ ID NO: 557) | TTTGCAGTCCTCTCAGCATAGAACTCAATGCGATCGGTATTTA |
| | TCTTGCTCCCAATCACGACCCAAGCCCCTCCACGTAGCTCTGC |
| | AGCCTTGGGGATATA |
| 65 (SEQ ID NO: 558) | TTTGCAGTCCTCTCAGCATAGAACTCAATGCGATCTGTATTTA |
| | TCTTGCTACCAATCACGACCCAAGCCCCTCCACGTAGCTCTGC |
| | AGCCTTGGGGATATA |
| 66 (SEQ ID NO: 559) | TTTGCAGTCCTCTCAGCATAGAACTCAATGCGATCTGTATTTA |
| | TCTTGCTGCCAATCACGACCCAAGCCCCTCCACGTAGCTCTGC |
| | AGCCTTGGGGATATA |
| 67 (SEQ ID NO: 560) | TTTGCAGTCCTCTCAGCATAGAACTCAATGCGATCTGTATTTA |
| | TCTTGCTTCCAATCACGACCCAAGCCCCTCCACGTAGCTCTGC |
| | AGCCTTGGGGATATA |
| 68 (SEQ ID NO: 561) | TTTGCAGTCCTCTCAGCATAGAACTCAATGCGATCTGTATTTA |
| | TCTTGCTCCCAATCACGACCCAAGCCCCTCCACGTAGCTCTGC |
| | AGCCTTGGGGATATA |
| 69 (SEQ ID NO: 562) | TTTGCAGTCCTCTCAGCATAGAACTCAATGCGATCCGTATTTA |
| | TCTTGCTACCAATCACGACCCAAGCCCCTCCACGTAGCTCTGC |
| | AGCCTTGGGGATATA |
| 70 (SEQ ID NO: 563) | TTTGCAGTCCTCTCAGCATAGAACTCAATGCGATCCGTATTTA |
| | TCTTGCTGCCAATCACGACCCAAGCCCCTCCACGTAGCTCTGC |
| | AGCCTTGGGGATATA |
| 71 (SEQ ID NO: 564) | TTTGCAGTCCTCTCAGCATAGAACTCAATGCGATCCGTATTTA |
| | TCTTGCTTCCAATCACGACCCAAGCCCCTCCACGTAGCTCTGC |
| | AGCCTTGGGGATATA |
| 72 (SEQ ID NO: 565) | TTTGCAGTCCTCTCAGCATAGAACTCAATGCGATCCGTATTTA |
| | TCTTGCTCCCAATCACGACCCAAGCCCCTCCACGTAGCTCTGC |
| | AGCCTTGGGGATATA |
| 73 (SEQ ID NO: 566) | CTGACCTGAACTTGATCTCGATCAACCCTTGAGGTTCGAGAAC |
| | ATTGCCCTTTGCAGTCCTCTCAGCATAGAACTCAATGCGATCT |
| | GGATTTATCTTGCTACCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 74 (SEQ ID NO: 567) | CTGACCTGAACTTGATCTCGATCAACCCTTGAGGTTCGAGAAC |
| | ATTGCCCTTTGCAGTCCTCTCAGCATAGAACTCAATGCGATCT |
| | GGATTTATCTTGCTGCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 75 (SEQ ID NO: 568) | CTGACCTGAACTTGATCTCGATCAACCCTTGAGGTTCGAGAAC |
| | ATTGCCCTTTGCAGTCCTCTCAGCATAGAACTCAATGCGATCT |
| | GGATTTATCTTGCTTCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 76 (SEQ ID NO: 569) | CTGACCTGAACTTGATCTCGATCAACCCTTGAGGTTCGAGAAC |
| | ATTGCCCTTTGCAGTCCTCTCAGCATAGAACTCAATGCGATCT |
| | GGATTTATCTTGCTCCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 77 (SEQ ID NO: 570) | CTGACCTGAACTTGATCTCGATCAACCCTTGAGGTTCGAGAAC |
| | ATTGCCCTTTGCAGTCCTCTCAGCATAGAACTCAATGCGATCA |
| | GTATTTATCTTGCTATCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 78 (SEQ ID NO: 571) | CTGACCTGAACTTGATCTCGATCAACCCTTGAGGTTCGAGAAC |
| | ATTGCCCTTTGCAGTCCTCTCAGCATAGAACTCAATGCGATCG |
| | GTATTTATCTTGCTATCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 79 (SEQ ID NO: 572) | CTGACCTGAACTTGATCTCGATCAACCCTTGAGGTTCGAGAAC |
| | ATTGCCCTTTGCAGTCCTCTCAGCATAGAACTCAATGCGATCT |
| | GTATTTATCTTGCTATCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 80 (SEQ ID NO: 573) | CTGACCTGAACTTGATCTCGATCAACCCTTGAGGTTCGAGAAC |
| | ATTGCCCTTTGCAGTCCTCTCAGCATAGAACTCAATGCGATCC |
| | GTATTTATCTTGCTATCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 81 (SEQ ID NO: 574) | CTGACCTGAACTTGATCTCGATCAACCCTTGAGGTTCGAGAAC |
| | ATTGCCCTTTGCAGTCCTCTCAGCATAGAACTCAATGCGATCA |
| | GTATTTATCTTGCTACCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 82 (SEQ ID NO: 575) | CTGACCTGAACTTGATCTCGATCAACCCTTGAGGTTCGAGAAC |
| | ATTGCCCTTTGCAGTCCTCTCAGCATAGAACTCAATGCGATCA |
| | GTATTTATCTTGCTGCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 83 (SEQ ID NO: 576) | CTGACCTGAACTTGATCTCGATCAACCCTTGAGGTTCGAGAAC |
| | ATTGCCCTTTGCAGTCCTCTCAGCATAGAACTCAATGCGATCA |
| | GTATTTATCTTGCTTCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 84 (SEQ ID NO: 577) | CTGACCTGAACTTGATCTCGATCAACCCTTGAGGTTCGAGAAC |
| | ATTGCCCTTTGCAGTCCTCTCAGCATAGAACTCAATGCGATCA |
| | GTATTTATCTTGCTCCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 85 (SEQ ID NO: 578) | CTGACCTGAACTTGATCTCGATCAACCCTTGAGGTTCGAGAAC |
| | ATTGCCCTTTGCAGTCCTCTCAGCATAGAACTCAATGCGATCG |
| | GTATTTATCTTGCTACCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 86 (SEQ ID NO: 579) | CTGACCTGAACTTGATCTCGATCAACCCTTGAGGTTCGAGAAC |
| | ATTGCCCTTTGCAGTCCTCTCAGCATAGAACTCAATGCGATCG |
| | GTATTTATCTTGCTGCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 87 (SEQ ID NO: 580) | CTGACCTGAACTTGATCTCGATCAACCCTTGAGGTTCGAGAAC |
| | ATTGCCCTTTGCAGTCCTCTCAGCATAGAACTCAATGCGATCG |
| | GTATTTATCTTGCTTCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 88 (SEQ ID NO: 581) | CTGACCTGAACTTGATCTCGATCAACCCTTGAGGTTCGAGAAC |
| | ATTGCCCTTTGCAGTCCTCTCAGCATAGAACTCAATGCGATCG |
| | GTATTTATCTTGCTCCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 89 (SEQ ID NO: 582) | CTGACCTGAACTTGATCTCGATCAACCCTTGAGGTTCGAGAAC |
| | ATTGCCCTTTGCAGTCCTCTCAGCATAGAACTCAATGCGATCT |
| | GTATTTATCTTGCTACCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 90 (SEQ ID NO: 583) | CTGACCTGAACTTGATCTCGATCAACCCTTGAGGTTCGAGAAC |
| | ATTGCCCTTTGCAGTCCTCTCAGCATAGAACTCAATGCGATCT |
| | GTATTTATCTTGCTGCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 91 (SEQ ID NO: 584) | CTGACCTGAACTTGATCTCGATCAACCCTTGAGGTTCGAGAAC |
| | ATTGCCCTTTGCAGTCCTCTCAGCATAGAACTCAATGCGATCT |
| | GTATTTATCTTGCTTCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 92 (SEQ ID NO: 585) | CTGACCTGAACTTGATCTCGATCAACCCTTGAGGTTCGAGAAC |
| | ATTGCCCTTTGCAGTCCTCTCAGCATAGAACTCAATGCGATCT |
| | GTATTTATCTTGCTCCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 93 (SEQ ID NO: 586) | CTGACCTGAACTTGATCTCGATCAACCCTTGAGGTTCGAGAAC |
| | ATTGCCCTTTGCAGTCCTCTCAGCATAGAACTCAATGCGATCC |
| | GTATTTATCTTGCTACCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 94 (SEQ ID NO: 587) | CTGACCTGAACTTGATCTCGATCAACCCTTGAGGTTCGAGAAC |
| | ATTGCCCTTTGCAGTCCTCTCAGCATAGAACTCAATGCGATCC |
| | GTATTTATCTTGCTGCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 95 (SEQ ID NO: 588) | CTGACCTGAACTTGATCTCGATCAACCCTTGAGGTTCGAGAAC |
| | ATTGCCCTTTGCAGTCCTCTCAGCATAGAACTCAATGCGATCC |
| | GTATTTATCTTGCTTCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 96 (SEQ ID NO: 589) | CTGACCTGAACTTGATCTCGATCAACCCTTGAGGTTCGAGAAC |
| | ATTGCCCTTTGCAGTCCTCTCAGCATAGAACTCAATGCGATCC |
| | GTATTTATCTTGCTCCCAATCACGACCCAAGCCCCTCCACGTA |
| | GCTCTGCAGCCTTGGGGATATATACAAAGGCAGGCTGATTGT |
| | ATGTCCTAAGGTTCTCAACAATTGTTGACC |
| 97 (SEQ ID NO: 590) | TTTGCAGTCCTCTCAGCATAGAACTCAATGCGATCNGTATTTA |
| | TCTTGCTNCCAATCACGACCCAAGCCCCTCCACGTAGCTCTGC |
| | AGCCTTGGGGATATA |
| 98 (SEQ ID NO: 591) | TATATCCCCAAGGCTGCAGAGCTACGTGGAGGGGCTTGGGTC |
| | GTGATTGGTAGCAAGATAAATACAGATCGAATTGAGTTCTAT |
| | GCTGAGAGGACTGCAAA |
| 99 (SEQ ID NO: 592) | TTTGCAGTCCTCTCAGCATAGAACTCAATTCGATCTGTATTTA |
| | TCTTGCTACCAATCACGACCCAAGCCCCTCCACGTAGCTCTGC |
| | AGCCTTGGGGATATA |
| 100 (SEQ ID NO: 593) | TATATCCCCAAGGCTGCAGAGCTACGTGGAGGGGCTTGGGTC |
| | GTGATTGGNAGCAAGATAAATACNGATCGAATTGAGTTCTAT |
| | GCTGAGAGGACTGCAAA |
| 101 (SEQ ID NO: 594) | TTTGCAGTCCTCTCAGCATAGAACTCAATTCGATCNGTATTTA |
| | TCTTGCTNCCAATCACGACCCAAGCCCCTCCACGTAGCTCTGC |
| | AGCCTTGGGGATATA |

### Example 13

A method to introduce targeted mutations into a native corn Zea mays ACCase gene utilizes PEG delivery of CRISPR-Cas9/Cas12a and GRONs into protoplasts. Examples of target spacer sequences for sgRNA targeting the corn ACCase gene are shown, but not limited to, those listed in the below Table 16. Sequence information for exemplary GRONs is shown, but not limited to those listed in the below Table 17.

TABLE 16
Exemplary target spacer sequences for corn
ACCase sgRNA

| Sequence No. | 5′ to 3′ sequence |
|---|---|
| 1 (SEQ ID NO: 595) | ggttaattgaaatcaagttc |
| 2 (SEQ ID NO: 596) | ttgatttcaattaacccttg |
| 3 (SEQ ID NO: 597) | gtaatgttctcgaacctcaa |
| 4 (SEQ ID NO: 598) | ggtaatgttctcgaacctca |
| 5 (SEQ ID NO: 599) | aggttcgagaacattacctt |
| 6 (SEQ ID NO: 600) | tatgctgaaaggactgccaa |
| 7 (SEQ ID NO: 601) | gcattgagtgttatgctgaa |
| 8 (SEQ ID NO: 602) | tttcagcataacactcaatg |
| 9 (SEQ ID NO: 603) | gcataacactcaatgcggtc |
| 10 (SEQ ID NO: 604) | tcgtggaggagcttgggttg |
| 11 (SEQ ID NO: 605) | agagcttcgtggaggagctt |
| 12 (SEQ ID NO: 606) | gagagcttcgtggaggagct |
| 13 (SEQ ID NO: 607) | atggctggagagcttcgtgg |
| 14 (SEQ ID NO: 608) | cctatggctggagagcttcg |
| 15 (SEQ ID NO: 609) | ccacgaagctctccagccat |
| 16 (SEQ ID NO: 610) | tttgtgtacattcctatggc |
| 17 (SEQ ID NO: 611) | tgcttttgtgtacattccta |
| 18 (SEQ ID NO: 612) | ataggaatgtacacaaaagc |
| 19 (SEQ ID NO: 613) | caggctgattatatgtccta |
| 20 (SEQ ID NO: 614) | caacaattgtcgagaacctt |
| 21 (SEQ ID NO: 615) | aattaacccttgaggttcgagaac |
| 22 (SEQ ID NO: 616) | gcagtcctttcagcataacactca |
| 23 (SEQ ID NO: 617) | agcataacactcaatgcggtctgg |
| 24 (SEQ ID NO: 618) | ttttgctatcgaccacaacccaag |
| 25 (SEQ ID NO: 619) | gctatcgaccacaacccaagctcc |
| 26 (SEQ ID NO: 620) | ctatcgaccacaacccaagctcct |
| 27 (SEQ ID NO: 621) | tgtacattcctatggctggagagc |
| 28 (SEQ ID NO: 622) | gtgtacattcctatggctggagag |
| 29 (SEQ ID NO: 623) | gaacttgatttcaattaacc |
| 30 (SEQ ID NO: 624) | caagggttaattgaaatcaa |
| 31 (SEQ ID NO: 625) | ttgaggttcgagaacattac |
| 32 (SEQ ID NO: 626) | tgaggttcgagaacattacc |
| 33 (SEQ ID NO: 627) | aaggtaatgttctcgaacct |
| 34 (SEQ ID NO: 628) | ttggcagtcctttcagcata |
| 35 (SEQ ID NO: 629) | ttcagcataacactcaatgc |
| 36 (SEQ ID NO: 630) | cattgagtgttatgctgaaa |
| 37 (SEQ ID NO: 631) | gaccgcattgagtgttatgc |
| 38 (SEQ ID NO: 632) | caacccaagctcctccacga |
| 39 (SEQ ID NO: 633) | aagctcctccacgaagctct |
| 40 (SEQ ID NO: 634) | agctcctccacgaagctctc |
| 41 (SEQ ID NO: 635) | ccacgaagctctccagccat |
| 42 (SEQ ID NO: 636) | cgaagctctccagccatagg |
| 43 (SEQ ID NO: 637) | atggctggagagcttcgtgg |
| 44 (SEQ ID NO: 638) | gccataggaatgtacacaaa |
| 45 (SEQ ID NO: 639) | taggaatgtacacaaaagca |
| 46 (SEQ ID NO: 640) | gcttttgtgtacattcctat |
| 47 (SEQ ID NO: 641) | taggacatataatcagcctg |
| 48 (SEQ ID NO: 642) | aaggttctcgacaattgttg |
| 49 (SEQ ID NO: 643) | gttctcgaacctcaagggttaatt |
| 50 (SEQ ID NO: 644) | tgagtgttatgctgaaaggactgc |
| 51 (SEQ ID NO: 645) | ccagaccgcattgagtgttatgct |
| 52 (SEQ ID NO: 646) | cttgggttgtggtcgatagcaaaa |
| 53 (SEQ ID NO: 647) | ggagcttgggttgtggtcgatagc |
| 54 (SEQ ID NO: 648) | aggagcttgggttgtggtcgatag |
| 55 (SEQ ID NO: 649) | gctctccagccataggaatgtaca |
| 56 (SEQ ID NO: 650) | ctctccagccataggaatgtacac |
| 57 (SEQ ID NO: 651) | ggagcttcgtggaggagctt |

TABLE 17
Exemplary corn ACCase GRON Sequences

| Sequence No. | 5′ to 3′ (V = CY3; H = 3′DMT dC CPG) |
|---|---|
| 1 (SEQ ID NO: 652) | TACATTCCTATGGCTGGAGAGCTTCGTGGAGGAGCTTGGGTTGTG |
| | GTCGGTAGCAAAATAAATCCAGACCGCATTGAGTGTTATGCTGAA |
| | AGGACTGCCAA |
| 2 (SEQ ID NO: 653) | TACATTCCTATGGCTGGAGAGCTTCGTGGAGGAGCTTGGGTTGTG |
| | GTCGGCAGCAAAATAAATCCAGACCGCATTGAGTGTTATGCTGAA |
| | AGGACTGCCAA |
| 3 (SEQ ID NO: 654) | TACATTCCTATGGCTGGAGAGCTTCGTGGAGGAGCTTGGGTTGTG |
| | GTCGGAAGCAAAATAAATCCAGACCGCATTGAGTGTTATGCTGAA |
| | AGGACTGCCAA |
| 4 (SEQ ID NO: 655) | TACATTCCTATGGCTGGAGAGCTTCGTGGAGGAGCTTGGGTTGTG |
| | GTCGGGAGCAAAATAAATCCAGACCGCATTGAGTGTTATGCTGAA |
| | AGGACTGCCAA |
| 5 (SEQ ID NO: 656) | TACATTCCTATGGCTGGAGAGCTTCGTGGAGGAGCTTGGGTTGTG |
| | GTCGATAGCAAAATAAATACTGACCGCATTGAGTGTTATGCTGAA |
| | AGGACTGCCAA |
| 6 (SEQ ID NO: 657) | TACATTCCTATGGCTGGAGAGCTTCGTGGAGGAGCTTGGGTTGTG |
| | GTCGATAGCAAAATAAATACCGACCGCATTGAGTGTTATGCTGAA |
| | AGGACTGCCAA |
| 7 (SEQ ID NO: 658) | TACATTCCTATGGCTGGAGAGCTTCGTGGAGGAGCTTGGGTTGTG |
| | GTCGATAGCAAAATAAATACAGACCGCATTGAGTGTTATGCTGAA |
| | AGGACTGCCAA |
| 8 (SEQ ID NO: 659) | TACATTCCTATGGCTGGAGAGCTTCGTGGAGGAGCTTGGGTTGTG |
| | GTCGATAGCAAAATAAATACGGACCGCATTGAGTGTTATGCTGAA |
| | AGGACTGCCAA |
| 9 (SEQ ID NO: 660) | TACATTCCTATGGCTGGAGAGCTTCGTGGAGGAGCTTGGGTTGTG |
| | GTCGGTAGCAAAATAAATACTGACCGCATTGAGTGTTATGCTGAA |
| | AGGACTGCCAA |
| 10 (SEQ ID NO: 661) | TACATTCCTATGGCTGGAGAGCTTCGTGGAGGAGCTTGGGTTGTG |
| | GTCGGCAGCAAAATAAATACTGACCGCATTGAGTGTTATGCTGAA |
| | AGGACTGCCAA |
| 11 (SEQ ID NO: 662) | TACATTCCTATGGCTGGAGAGCTTCGTGGAGGAGCTTGGGTTGTG |
| | GTCGGAAGCAAAATAAATACTGACCGCATTGAGTGTTATGCTGAA |
| | AGGACTGCCAA |
| 12 (SEQ ID NO: 663) | TACATTCCTATGGCTGGAGAGCTTCGTGGAGGAGCTTGGGTTGTG |
| | GTCGGGAGCAAAATAAATACTGACCGCATTGAGTGTTATGCTGAA |
| | AGGACTGCCAA |
| 13 (SEQ ID NO: 664) | TACATTCCTATGGCTGGAGAGCTTCGTGGAGGAGCTTGGGTTGTG |
| | GTCGGTAGCAAAATAAATACCGACCGCATTGAGTGTTATGCTGAA |
| | AGGACTGCCAA |
| 14 (SEQ ID NO: 665) | TACATTCCTATGGCTGGAGAGCTTCGTGGAGGAGCTTGGGTTGTG |
| | GTCGGCAGCAAAATAAATACCGACCGCATTGAGTGTTATGCTGAA |
| | AGGACTGCCAA |
| 15 (SEQ ID NO: 666) | TACATTCCTATGGCTGGAGAGCTTCGTGGAGGAGCTTGGGTTGTG |
| | GTCGGAAGCAAAATAAATACCGACCGCATTGAGTGTTATGCTGAA |
| | AGGACTGCCAA |
| 16 (SEQ ID NO: 667) | TACATTCCTATGGCTGGAGAGCTTCGTGGAGGAGCTTGGGTTGTG |
| | GTCGGGAGCAAAATAAATACCGACCGCATTGAGTGTTATGCTGAA |
| | AGGACTGCCAA |
| 17 (SEQ ID NO: 668) | TACATTCCTATGGCTGGAGAGCTTCGTGGAGGAGCTTGGGTTGTG |
| | GTCGGTAGCAAAATAAATACAGACCGCATTGAGTGTTATGCTGAA |
| | AGGACTGCCAA |
| 18 (SEQ ID NO: 669) | TACATTCCTATGGCTGGAGAGCTTCGTGGAGGAGCTTGGGTTGTG |
| | GTCGGCAGCAAAATAAATACAGACCGCATTGAGTGTTATGCTGAA |
| | AGGACTGCCAA |
| 19 (SEQ ID NO: 670) | TACATTCCTATGGCTGGAGAGCTTCGTGGAGGAGCTTGGGTTGTG |
| | GTCGGAAGCAAAATAAATACAGACCGCATTGAGTGTTATGCTGA |
| | AAGGACTGCCAA |
| 20 (SEQ ID NO: 671) | TACATTCCTATGGCTGGAGAGCTTCGTGGAGGAGCTTGGGTTGTG |
| | GTCGGGAGCAAAATAAATACAGACCGCATTGAGTGTTATGCTGA |
| | AAGGACTGCCAA |
| 21 (SEQ ID NO: 672) | TACATTCCTATGGCTGGAGAGCTTCGTGGAGGAGCTTGGGTTGTG |
| | GTCGGTAGCAAAATAAATACGGACCGCATTGAGTGTTATGCTGAA |
| | AGGACTGCCAA |
| 22 (SEQ ID NO: 673) | TACATTCCTATGGCTGGAGAGCTTCGTGGAGGAGCTTGGGTTGTG |
| | GTCGGCAGCAAAATAAATACGGACCGCATTGAGTGTTATGCTGAA |
| | AGGACTGCCAA |
| 23 (SEQ ID NO: 674) | TACATTCCTATGGCTGGAGAGCTTCGTGGAGGAGCTTGGGTTGTG |
| | GTCGGAAGCAAAATAAATACGGACCGCATTGAGTGTTATGCTGA |
| | AAGGACTGCCAA |
| 24 (SEQ ID NO: 675) | TACATTCCTATGGCTGGAGAGCTTCGTGGAGGAGCTTGGGTTGTG |
| | GTCGGGAGCAAAATAAATACGGACCGCATTGAGTGTTATGCTGA |
| | AAGGACTGCCAA |
| 25 (SEQ ID NO: 676) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGTAGCAAAATAAATCCAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 26 (SEQ ID NO: 677) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGCAGCAAAATAAATCCAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 27 (SEQ ID NO: 678) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGAAGCAAAATAAATCCAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 28 (SEQ ID NO: 679) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGGAGCAAAATAAATCCAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 29 (SEQ ID NO: 680) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGATAGCAAAATAAATACTGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 30 (SEQ ID NO: 681) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGATAGCAAAATAAATACCGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 31 (SEQ ID NO: 682) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGATAGCAAAATAAATACAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 32 (SEQ ID NO: 683) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGATAGCAAAATAAATACGGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 33 (SEQ ID NO: 684) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGTAGCAAAATAAATACTGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 34 (SEQ ID NO: 685) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGCAGCAAAATAAATACTGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 35 (SEQ ID NO: 686) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGAAGCAAAATAAATACTGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 36 (SEQ ID NO: 687) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGGAGCAAAATAAATACTGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 37 (SEQ ID NO: 688) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGTAGCAAAATAAATACCGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 38 (SEQ ID NO: 689) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGCAGCAAAATAAATACCGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 39 (SEQ ID NO: 690) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGAAGCAAAATAAATACCGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 40 (SEQ ID NO: 691) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGGAGCAAAATAAATACCGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 41 (SEQ ID NO: 692) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGTAGCAAAATAAATACAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 42 (SEQ ID NO: 693) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGCAGCAAAATAAATACAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 43 (SEQ ID NO: 694) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGAAGCAAAATAAATACAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 44 (SEQ ID NO: 695) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGGAGCAAAATAAATACAGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 45 (SEQ ID NO: 696) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGTAGCAAAATAAATACGGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 46 (SEQ ID NO: 697) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGCAGCAAAATAAATACGGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 47 (SEQ ID NO: 698) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGAAGCAAAATAAATACGGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 48 (SEQ ID NO: 699) | GGTCAACAATTGTCGAGAACCTTAGGACATATAATCAGCCTGCTT |
| | TTGTGTACATTCCTATGGCTGGAGAGCTTCGTGGAGGAGCTTGGG |
| | TTGTGGTCGGGAGCAAAATAAATACGGACCGCATTGAGTGTTATG |
| | CTGAAAGGACTGCCAAAGGTAATGTTCTCGAACCTCAAGGGTTAA |
| | TTGAAATCAAGTTCAGGTCAG |
| 49 (SEQ ID NO: 700) | TTGGCAGTCCTTTCAGCATAACACTCAATGCGGTCTGGATTTATTT |
| | TGCTACCGACCACAACCCAAGCTCCTCCACGAAGCTCTCCAGCCA |
| | TAGGAATGTA |
| 50 (SEQ ID NO: 701) | TTGGCAGTCCTTTCAGCATAACACTCAATGCGGTCTGGATTTATTT |
| | TGCTGCCGACCACAACCCAAGCTCCTCCACGAAGCTCTCCAGCCA |
| | TAGGAATGTA |
| 51 (SEQ ID NO: 702) | TTGGCAGTCCTTTCAGCATAACACTCAATGCGGTCTGGATTTATTT |
| | TGCTTCCGACCACAACCCAAGCTCCTCCACGAAGCTCTCCAGCCA |
| | TAGGAATGTA |
| 52 (SEQ ID NO: 703) | TTGGCAGTCCTTTCAGCATAACACTCAATGCGGTCTGGATTTATTT |
| | TGCTCCCGACCACAACCCAAGCTCCTCCACGAAGCTCTCCAGCCA |
| | TAGGAATGTA |
| 53 (SEQ ID NO: 704) | TTGGCAGTCCTTTCAGCATAACACTCAATGCGGTCAGTATTTATTT |
| | TGCTATCGACCACAACCCAAGCTCCTCCACGAAGCTCTCCAGCCA |
| | TAGGAATGTA |
| 54 (SEQ ID NO: 705) | TTGGCAGTCCTTTCAGCATAACACTCAATGCGGTCGGTATTTATTT |
| | TGCTATCGACCACAACCCAAGCTCCTCCACGAAGCTCTCCAGCCA |
| | TAGGAATGTA |
| 55 (SEQ ID NO: 706) | TTGGCAGTCCTTTCAGCATAACACTCAATGCGGTCTGTATTTATTT |
| | TGCTATCGACCACAACCCAAGCTCCTCCACGAAGCTCTCCAGCCA |
| | TAGGAATGTA |
| 56 (SEQ ID NO: 707) | TTGGCAGTCCTTTCAGCATAACACTCAATGCGGTCCGTATTTATTT |
| | TGCTATCGACCACAACCCAAGCTCCTCCACGAAGCTCTCCAGCCA |
| | TAGGAATGTA |
| 57 (SEQ ID NO: 708) | TTGGCAGTCCTTTCAGCATAACACTCAATGCGGTCAGTATTTATTT |
| | TGCTACCGACCACAACCCAAGCTCCTCCACGAAGCTCTCCAGCCA |
| | TAGGAATGTA |
| 58 (SEQ ID NO: 709) | TTGGCAGTCCTTTCAGCATAACACTCAATGCGGTCAGTATTTATTT |
| | TGCTGCCGACCACAACCCAAGCTCCTCCACGAAGCTCTCCAGCCA |
| | TAGGAATGTA |
| 59 (SEQ ID NO: 710) | TTGGCAGTCCTTTCAGCATAACACTCAATGCGGTCAGTATTTATTT |
| | TGCTTCCGACCACAACCCAAGCTCCTCCACGAAGCTCTCCAGCCA |
| | TAGGAATGTA |
| 60 (SEQ ID NO: 711) | TTGGCAGTCCTTTCAGCATAACACTCAATGCGGTCAGTATTTATTT |
| | TGCTCCCGACCACAACCCAAGCTCCTCCACGAAGCTCTCCAGCCA |
| | TAGGAATGTA |
| 61 (SEQ ID NO: 712) | TTGGCAGTCCTTTCAGCATAACACTCAATGCGGTCGGTATTTATTT |
| | TGCTACCGACCACAACCCAAGCTCCTCCACGAAGCTCTCCAGCCA |
| | TAGGAATGTA |
| 62 (SEQ ID NO: 713) | TTGGCAGTCCTTTCAGCATAACACTCAATGCGGTCGGTATTTATTT |
| | TGCTGCCGACCACAACCCAAGCTCCTCCACGAAGCTCTCCAGCCA |
| | TAGGAATGTA |
| 63 (SEQ ID NO: 714) | TTGGCAGTCCTTTCAGCATAACACTCAATGCGGTCGGTATTTATTT |
| | TGCTTCCGACCACAACCCAAGCTCCTCCACGAAGCTCTCCAGCCA |
| | TAGGAATGTA |
| 64 (SEQ ID NO: 715) | TTGGCAGTCCTTTCAGCATAACACTCAATGCGGTCGGTATTTATTT |
| | TGCTCCCGACCACAACCCAAGCTCCTCCACGAAGCTCTCCAGCCA |
| | TAGGAATGTA |
| 65 (SEQ ID NO: 716) | TTGGCAGTCCTTTCAGCATAACACTCAATGCGGTCTGTATTTATTT |
| | TGCTACCGACCACAACCCAAGCTCCTCCACGAAGCTCTCCAGCCA |
| | TAGGAATGTA |
| 66 (SEQ ID NO: 717) | TTGGCAGTCCTTTCAGCATAACACTCAATGCGGTCTGTATTTATTT |
| | TGCTGCCGACCACAACCCAAGCTCCTCCACGAAGCTCTCCAGCCA |
| | TAGGAATGTA |
| 67 (SEQ ID NO: 718) | TTGGCAGTCCTTTCAGCATAACACTCAATGCGGTCTGTATTTATTT |
| | TGCTTCCGACCACAACCCAAGCTCCTCCACGAAGCTCTCCAGCCA |
| | TAGGAATGTA |
| 68 (SEQ ID NO: 719) | TTGGCAGTCCTTTCAGCATAACACTCAATGCGGTCTGTATTTATTT |
| | TGCTCCCGACCACAACCCAAGCTCCTCCACGAAGCTCTCCAGCCA |
| | TAGGAATGTA |
| 69 (SEQ ID NO: 720) | TTGGCAGTCCTTTCAGCATAACACTCAATGCGGTCCGTATTTATTT |
| | TGCTACCGACCACAACCCAAGCTCCTCCACGAAGCTCTCCAGCCA |
| | TAGGAATGTA |
| 70 (SEQ ID NO: 721) | TTGGCAGTCCTTTCAGCATAACACTCAATGCGGTCCGTATTTATTT |
| | TGCTGCCGACCACAACCCAAGCTCCTCCACGAAGCTCTCCAGCCA |
| | TAGGAATGTA |
| 71 (SEQ ID NO: 722) | TTGGCAGTCCTTTCAGCATAACACTCAATGCGGTCCGTATTTATTT |
| | TGCTTCCGACCACAACCCAAGCTCCTCCACGAAGCTCTCCAGCCA |
| | TAGGAATGTA |
| 72 (SEQ ID NO: 723) | TTGGCAGTCCTTTCAGCATAACACTCAATGCGGTCCGTATTTATTT |
| | TGCTCCCGACCACAACCCAAGCTCCTCCACGAAGCTCTCCAGCCA |
| | TAGGAATGTA |
| 73 (SEQ ID NO: 724) | CTGACCTGAACTTGATTTCAATTAACCCTTGAGGTTCGAGAACAT |
| | TACCTTTGGCAGTCCTTTCAGCATAACACTCAATGCGGTCTGGATT |
| | TATTTTGCTACCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 74 (SEQ ID NO: 725) | CTGACCTGAACTTGATTTCAATTAACCCTTGAGGTTCGAGAACAT |
| | TACCTTTGGCAGTCCTTTCAGCATAACACTCAATGCGGTCTGGATT |
| | TATTTTGCTGCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 75 (SEQ ID NO: 726) | CTGACCTGAACTTGATTTCAATTAACCCTTGAGGTTCGAGAACAT |
| | TACCTTTGGCAGTCCTTTCAGCATAACACTCAATGCGGTCTGGATT |
| | TATTTTGCTTCCGACCACAACCCAAGCTCCTCCACGAAGCTCTCCA |
| | GCCATAGGAATGTACACAAAAGCAGGCTGATTATATGTCCTAAGG |
| | TTCTCGACAATTGTTGACC |
| 76 (SEQ ID NO: 727) | CTGACCTGAACTTGATTTCAATTAACCCTTGAGGTTCGAGAACAT |
| | TACCTTTGGCAGTCCTTTCAGCATAACACTCAATGCGGTCTGGATT |
| | TATTTTGCTCCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 77 (SEQ ID NO: 728) | CTGACCTGAACTTGATTTCAATTAACCCTTGAGGTTCGAGAACAT |
| | TACCTTTGGCAGTCCTTTCAGCATAACACTCAATGCGGTCAGTATT |
| | TATTTTGCTATCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 78 (SEQ ID NO: 729) | CTGACCTGAACTTGATTTCAATTAACCCTTGAGGTTCGAGAACAT |
| | TACCTTTGGCAGTCCTTTCAGCATAACACTCAATGCGGTCGGTATT |
| | TATTTTGCTATCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 79 (SEQ ID NO: 730) | CTGACCTGAACTTGATTTCAATTAACCCTTGAGGTTCGAGAACAT |
| | TACCTTTGGCAGTCCTTTCAGCATAACACTCAATGCGGTCTGTATT |
| | TATTTTGCTATCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 80 (SEQ ID NO: 731) | CTGACCTGAACTTGATTTCAATTAACCCTTGAGGTTCGAGAACAT |
| | TACCTTTGGCAGTCCTTTCAGCATAACACTCAATGCGGTCCGTATT |
| | TATTTTGCTATCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 81 (SEQ ID NO: 732) | CTGACCTGAACTTGATTTCAATTAACCCTTGAGGTTCGAGAACAT |
| | TACCTTTGGCAGTCCTTTCAGCATAACACTCAATGCGGTCAGTATT |
| | TATTTTGCTACCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 82 (SEQ ID NO: 733) | CTGACCTGAACTTGATTTCAATTAACCCTTGAGGTTCGAGAACAT |
| | TACCTTTGGCAGTCCTTTCAGCATAACACTCAATGCGGTCAGTATT |
| | TATTTTGCTGCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 83 (SEQ ID NO: 734) | CTGACCTGAACTTGATTTCAATTAACCCTTGAGGTTCGAGAACAT |
| | TACCTTTGGCAGTCCTTTCAGCATAACACTCAATGCGGTCAGTATT |
| | TATTTTGCTTCCGACCACAACCCAAGCTCCTCCACGAAGCTCTCCA |
| | GCCATAGGAATGTACACAAAAGCAGGCTGATTATATGTCCTAAGG |
| | TTCTCGACAATTGTTGACC |
| 84 (SEQ ID NO: 735) | CTGACCTGAACTTGATTTCAATTAACCCTTGAGGTTCGAGAACAT |
| | TACCTTTGGCAGTCCTTTCAGCATAACACTCAATGCGGTCAGTATT |
| | TATTTTGCTCCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 85 (SEQ ID NO: 736) | CTGACCTGAACTTGATTTCAATTAACCCTTGAGGTTCGAGAACAT |
| | TACCTTTGGCAGTCCTTTCAGCATAACACTCAATGCGGTCGGTATT |
| | TATTTTGCTACCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 86 (SEQ ID NO: 737) | CTGACCTGAACTTGATTTCAATTAACCCTTGAGGTTCGAGAACAT |
| | TACCTTTGGCAGTCCTTTCAGCATAACACTCAATGCGGTCGGTATT |
| | TATTTTGCTGCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 87 (SEQ ID NO: 738) | CTGACCTGAACTTGATTTCAATTAACCCTTGAGGTTCGAGAACAT |
| | TACCTTTGGCAGTCCTTTCAGCATAACACTCAATGCGGTCGGTATT |
| | TATTTTGCTTCCGACCACAACCCAAGCTCCTCCACGAAGCTCTCCA |
| | GCCATAGGAATGTACACAAAAGCAGGCTGATTATATGTCCTAAGG |
| | TTCTCGACAATTGTTGACC |
| 88 (SEQ ID NO: 739) | CTGACCTGAACTTGATTTCAATTAACCCTTGAGGTTCGAGAACAT |
| | TACCTTTGGCAGTCCTTTCAGCATAACACTCAATGCGGTCGGTATT |
| | TATTTTGCTCCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 89 (SEQ ID NO: 740) | CTGACCTGAACTTGATTTCAATTAACCCTTGAGGTTCGAGAACAT |
| | TACCTTTGGCAGTCCTTTCAGCATAACACTCAATGCGGTCTGTATT |
| | TATTTTGCTACCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 90 (SEQ ID NO: 741) | CTGACCTGAACTTGATTTCAATTAACCCTTGAGGTTCGAGAACAT |
| | TACCTTTGGCAGTCCTTTCAGCATAACACTCAATGCGGTCTGTATT |
| | TATTTTGCTGCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 91 (SEQ ID NO: 742) | CTGACCTGAACTTGATTTCAATTAACCCTTGAGGTTCGAGAACAT |
| | TACCTTTGGCAGTCCTTTCAGCATAACACTCAATGCGGTCTGTATT |
| | TATTTTGCTTCCGACCACAACCCAAGCTCCTCCACGAAGCTCTCCA |
| | GCCATAGGAATGTACACAAAAGCAGGCTGATTATATGTCCTAAGG |
| | TTCTCGACAATTGTTGACC |
| 92 (SEQ ID NO: 743) | CTGACCTGAACTTGATTTCAATTAACCCTTGAGGTTCGAGAACAT |
| | TACCTTTGGCAGTCCTTTCAGCATAACACTCAATGCGGTCTGTATT |
| | TATTTTGCTCCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 93 (SEQ ID NO: 744) | CTGACCTGAACTTGATTTCAATTAACCCTTGAGGTTCGAGAACAT |
| | TACCTTTGGCAGTCCTTTCAGCATAACACTCAATGCGGTCCGTATT |
| | TATTTTGCTACCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 94 (SEQ ID NO: 745) | CTGACCTGAACTTGATTTCAATTAACCCTTGAGGTTCGAGAACAT |
| | TACCTTTGGCAGTCCTTTCAGCATAACACTCAATGCGGTCCGTATT |
| | TATTTTGCTGCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 95 (SEQ ID NO: 746) | CTGACCTGAACTTGATTTCAATTAACCCTTGAGGTTCGAGAACAT |
| | TACCTTTGGCAGTCCTTTCAGCATAACACTCAATGCGGTCCGTATT |
| | TATTTTGCTTCCGACCACAACCCAAGCTCCTCCACGAAGCTCTCCA |
| | GCCATAGGAATGTACACAAAAGCAGGCTGATTATATGTCCTAAGG |
| | TTCTCGACAATTGTTGACC |
| 96 (SEQ ID NO: 747) | CTGACCTGAACTTGATTTCAATTAACCCTTGAGGTTCGAGAACAT |
| | TACCTTTGGCAGTCCTTTCAGCATAACACTCAATGCGGTCCGTATT |
| | TATTTTGCTCCCGACCACAACCCAAGCTCCTCCACGAAGCTCTCC |
| | AGCCATAGGAATGTACACAAAAGCAGGCTGATTATATGTCCTAAG |
| | GTTCTCGACAATTGTTGACC |
| 97 (SEQ ID NO: 748) | TACATTCCTATGGCTGGAGAGCTTCGTGGAGGAGCTTGGGTTGTG |
| | ATCGGTAGCAAAATAAATACAGACCGCATTGAGTGTTATGCTGAG |
| | AGGACTGCCAA |
| 98 (SEQ ID NO: 749) | TTGGCAGTCCTCTCAGCATAACACTCAATGCGGTCTGTATTTATTT |
| | TGCTACCGATCACAACCCAAGCTCCTCCACGAAGCTCTCCAGCCA |
| | TAGGAATGTA |
| 99 (SEQ ID NO: 750) | TACATTCCTATGGCTGGAGAGCTTCGTGGAGGAGCTTGGGTTGTG |
| | ATCGGNAGCAAAATAAATACNGACCGCATTGAGTGTTATGCTGA |
| | GAGGACTGCCAA |
| 100 (SEQ ID NO: 751) | TTGGCAGTCCTCTCAGCATAACACTCAATGCGGTCNGTATTTATTT |
| | TGCTNCCGATCACAACCCAAGCTCCTCCACGAAGCTCTCCAGCCA |
| | TAGGAATGTA |
| 101 (SEQ ID NO: 752) | TACATTCCTATGGCTGGAGAGCTTCGTGGAGGAGCTTGGGTTGTG |
| | GTCGGNAGCAAAATAAATACNGACCGCATTGAGTGTTATGCTGA |
| | AAGGACTGCCAA |
| 102 (SEQ ID NO: 753) | TTGGCAGTCCTTTCAGCATAACACTCAATGCGGTCNGTATTTATTT |
| | TGCTNCCGACCACAACCCAAGCTCCTCCACGAAGCTCTCCAGCCA |
| | TAGGAATGTA |

### Example 14

A method to introduce targeted mutations into a native sorghum Sorghum bicolor ACCase gene utilizes PEG delivery of CRISPR-Cas9/Cas12a and GRONs into protoplasts. Examples of target spacer sequences for sgRNA targeting the sorghum ACCase gene are shown, but not limited to, those listed in the below Table 18. Sequence information for exemplary GRONs is shown, but not limited to those listed in the below Table 19.

TABLE 18
Exemplary target spacer sequences for sorghum ACCase sgRNA

| Sequence No. | 5′ to 3′ sequence |
|---|---|
| 1 (SEQ ID NO: 754) | ggttaattgaaatcaagttc |
| 2 (SEQ ID NO: 755) | ttgatttcaattaacccttg |
| 3 (SEQ ID NO: 756) | gtaatgttctcgaacctcaa |
| 4 (SEQ ID NO: 757) | ggtaatgttctcgaacctca |
| 5 (SEQ ID NO: 758) | aggttcgagaacattacctt |
| 6 (SEQ ID NO: 759) | tatgctgagaggactgccaa |
| 7 (SEQ ID NO: 760) | gcattgagtgttatgctgag |
| 8 (SEQ ID NO: 761) | tctcagcataacactcaatg |
| 9 (SEQ ID NO: 762) | gcataacactcaatgcggtc |
| 10 (SEQ ID NO: 763) | tcgtggaggagcttgggttg |
| 11 (SEQ ID NO: 764) | agagcttcgtggaggagctt |
| 12 (SEQ ID NO: 765) | gagagcttcgtggaggagct |
| 13 (SEQ ID NO: 766) | atggctggagagcttcgtgg |
| 14 (SEQ ID NO: 767) | cctatggctggagagcttcg |
| 15 (SEQ ID NO: 768) | ccacgaagctctccagccat |
| 16 (SEQ ID NO: 769) | tttgtctacattcctatggc |
| 17 (SEQ ID NO: 770) | tgcgtttgtctacattccta |
| 18 (SEQ ID NO: 771) | ataggaatgtagacaaacgc |
| 19 (SEQ ID NO: 772) | caggctgattatatgtccta |
| 20 (SEQ ID NO: 773) | caacaattgtcgagaacctt |
| 21 (SEQ ID NO: 774) | aattaacccttgaggttcgagaac |
| 22 (SEQ ID NO: 775) | gcagtcctctcagcataacactca |
| 23 (SEQ ID NO: 776) | ttttgctatcgaccacaacccaag |
| 24 (SEQ ID NO: 777) | gctatcgaccacaacccaagctcc |
| 25 (SEQ ID NO: 778) | ctatcgaccacaacccaagctcct |
| 26 (SEQ ID NO: 779) | tctacattcctatggctggagagc |
| 27 (SEQ ID NO: 780) | gaacttgatttcaattaacc |
| 28 (SEQ ID NO: 781) | caagggttaattgaaatcaa |
| 29 (SEQ ID NO: 782) | ttgaggttcgagaacattac |
| 30 (SEQ ID NO: 783) | tgaggttcgagaacattacc |
| 31 (SEQ ID NO: 784) | aaggtaatgttctcgaacct |
| 32 (SEQ ID NO: 785) | ttggcagtcctctcagcata |
| 33 (SEQ ID NO: 786) | ctcagcataacactcaatgc |
| 34 (SEQ ID NO: 787) | cattgagtgttatgctgaga |
| 35 (SEQ ID NO: 788) | gaccgcattgagtgttatgc |
| 36 (SEQ ID NO: 789) | caacccaagctcctccacga |
| 37 (SEQ ID NO: 790) | aagctcctccacgaagctct |
| 38 (SEQ ID NO: 791) | agctcctccacgaagctctc |
| 39 (SEQ ID NO: 792) | ccacgaagctctccagccat |
| 40 (SEQ ID NO: 793) | cgaagctctccagccatagg |
| 41 (SEQ ID NO: 794) | atggctggagagcttcgtgg |
| 42 (SEQ ID NO: 795) | gccataggaatgtagacaaa |
| 43 (SEQ ID NO: 796) | taggaatgtagacaaacgca |
| 44 (SEQ ID NO: 797) | gcgtttgtctacattcctat |
| 45 (SEQ ID NO: 798) | taggacatataatcagcctg |
| 46 (SEQ ID NO: 799) | aaggttctcgacaattgttg |
| 47 (SEQ ID NO: 800) | gttctcgaacctcaagggttaatt |
| 48 (SEQ ID NO: 801) | tgagtgttatgctgagaggactgc |
| 49 (SEQ ID NO: 802) | cttgggttgtggtcgatagcaaaa |
| 50 (SEQ ID NO: 803) | ggagcttgggttgtggtcgatagc |
| 51 (SEQ ID NO: 804) | aggagcttgggttgtggtcgatag |
| 52 (SEQ ID NO: 805) | gctctccagccataggaatgtaga |

TABLE 19
Exemplary sorghum ACCase GRON Sequences

| Sequence No. | 5′ to 3′ (V = CY3; H = 3′DMT dC CPG) |
|---|---|
| 1 (SEQ ID NO: 806) | TACATTCCTATGGCTGGAGAGCTTCGTGGAGGAGCTTGGGTTGT |
| | GGTCGGTAGCAAAATAAATCCAGACCGCATTGAGTGTTATGCTG |
| | AGAGGACTGCCAA |
| 2 (SEQ ID NO: 807) | TACATTCCTATGGCTGGAGAGCTTCGTGGAGGAGCTTGGGTTGT |
| | GGTCGGCAGCAAAATAAATCCAGACCGCATTGAGTGTTATGCTG |
| | AGAGGACTGCCAA |
| 3 (SEQ ID NO: 808) | TACATTCCTATGGCTGGAGAGCTTCGTGGAGGAGCTTGGGTTGT |
| | GGTCGGAAGCAAAATAAATCCAGACCGCATTGAGTGTTATGCTG |
| | AGAGGACTGCCAA |
| 4 (SEQ ID NO: 809) | TACATTCCTATGGCTGGAGAGCTTCGTGGAGGAGCTTGGGTTGT |
| | GGTCGGGAGCAAAATAAATCCAGACCGCATTGAGTGTTATGCTG |
| | AGAGGACTGCCAA |
| 5 (SEQ ID NO: 810) | TACATTCCTATGGCTGGAGAGCTTCGTGGAGGAGCTTGGGTTGT |
| | GGTCGATAGCAAAATAAATACTGACCGCATTGAGTGTTATGCTG |
| | AGAGGACTGCCAA |
| 6 (SEQ ID NO: 811) | TACATTCCTATGGCTGGAGAGCTTCGTGGAGGAGCTTGGGTTGT |
| | GGTCGATAGCAAAATAAATACCGACCGCATTGAGTGTTATGCTG |
| | AGAGGACTGCCAA |
| 7 (SEQ ID NO: 812) | TACATTCCTATGGCTGGAGAGCTTCGTGGAGGAGCTTGGGTTGT |
| | GGTCGATAGCAAAATAAATACAGACCGCATTGAGTGTTATGCTG |
| | AGAGGACTGCCAA |
| 8 (SEQ ID NO: 813) | TACATTCCTATGGCTGGAGAGCTTCGTGGAGGAGCTTGGGTTGT |
| | GGTCGATAGCAAAATAAATACGGACCGCATTGAGTGTTATGCTG |
| | AGAGGACTGCCAA |
| 9 (SEQ ID NO: 814) | TACATTCCTATGGCTGGAGAGCTTCGTGGAGGAGCTTGGGTTGT |
| | GGTCGGTAGCAAAATAAATACTGACCGCATTGAGTGTTATGCTG |
| | AGAGGACTGCCAA |
| 10 (SEQ ID NO: 815) | TACATTCCTATGGCTGGAGAGCTTCGTGGAGGAGCTTGGGTTGT |
| | GGTCGGCAGCAAAATAAATACTGACCGCATTGAGTGTTATGCTG |
| | AGAGGACTGCCAA |
| 11 (SEQ ID NO: 816) | TACATTCCTATGGCTGGAGAGCTTCGTGGAGGAGCTTGGGTTGT |
| | GGTCGGAAGCAAAATAAATACTGACCGCATTGAGTGTTATGCTG |
| | AGAGGACTGCCAA |
| 12 (SEQ ID NO: 817) | TACATTCCTATGGCTGGAGAGCTTCGTGGAGGAGCTTGGGTTGT |
| | GGTCGGGAGCAAAATAAATACTGACCGCATTGAGTGTTATGCTG |
| | AGAGGACTGCCAA |
| 13 (SEQ ID NO: 818) | TACATTCCTATGGCTGGAGAGCTTCGTGGAGGAGCTTGGGTTGT |
| | GGTCGGTAGCAAAATAAATACCGACCGCATTGAGTGTTATGCTG |
| | AGAGGACTGCCAA |
| 14 (SEQ ID NO: 819) | TACATTCCTATGGCTGGAGAGCTTCGTGGAGGAGCTTGGGTTGT |
| | GGTCGGCAGCAAAATAAATACCGACCGCATTGAGTGTTATGCTG |
| | AGAGGACTGCCAA |
| 15 (SEQ ID NO: 820) | TACATTCCTATGGCTGGAGAGCTTCGTGGAGGAGCTTGGGTTGT |
| | GGTCGGAAGCAAAATAAATACCGACCGCATTGAGTGTTATGCTG |
| | AGAGGACTGCCAA |
| 16 (SEQ ID NO: 821) | TACATTCCTATGGCTGGAGAGCTTCGTGGAGGAGCTTGGGTTGT |
| | GGTCGGGAGCAAAATAAATACCGACCGCATTGAGTGTTATGCTG |
| | AGAGGACTGCCAA |
| 17 (SEQ ID NO: 822) | TACATTCCTATGGCTGGAGAGCTTCGTGGAGGAGCTTGGGTTGT |
| | GGTCGGTAGCAAAATAAATACAGACCGCATTGAGTGTTATGCTG |
| | AGAGGACTGCCAA |
| 18 (SEQ ID NO: 823) | TACATTCCTATGGCTGGAGAGCTTCGTGGAGGAGCTTGGGTTGT |
| | GGTCGGCAGCAAAATAAATACAGACCGCATTGAGTGTTATGCTG |
| | AGAGGACTGCCAA |
| 19 (SEQ ID NO: 824) | TACATTCCTATGGCTGGAGAGCTTCGTGGAGGAGCTTGGGTTGT |
| | GGTCGGAAGCAAAATAAATACAGACCGCATTGAGTGTTATGCTG |
| | AGAGGACTGCCAA |
| 20 (SEQ ID NO: 825) | TACATTCCTATGGCTGGAGAGCTTCGTGGAGGAGCTTGGGTTGT |
| | GGTCGGGAGCAAAATAAATACAGACCGCATTGAGTGTTATGCTG |
| | AGAGGACTGCCAA |
| 21 (SEQ ID NO: 826) | TACATTCCTATGGCTGGAGAGCTTCGTGGAGGAGCTTGGGTTGT |
| | GGTCGGTAGCAAAATAAATACGGACCGCATTGAGTGTTATGCTG |
| | AGAGGACTGCCAA |
| 22 (SEQ ID NO: 827) | TACATTCCTATGGCTGGAGAGCTTCGTGGAGGAGCTTGGGTTGT |
| | GGTCGGCAGCAAAATAAATACGGACCGCATTGAGTGTTATGCTG |
| | AGAGGACTGCCAA |
| 23 (SEQ ID NO: 828) | TACATTCCTATGGCTGGAGAGCTTCGTGGAGGAGCTTGGGTTGT |
| | GGTCGGAAGCAAAATAAATACGGACCGCATTGAGTGTTATGCTG |
| | AGAGGACTGCCAA |
| 24 (SEQ ID NO: 829) | TACATTCCTATGGCTGGAGAGCTTCGTGGAGGAGCTTGGGTTGT |
| | GGTCGGGAGCAAAATAAATACGGACCGCATTGAGTGTTATGCTG |
| | AGAGGACTGCCAA |
| 25 (SEQ ID NO: 830) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGTAGCAAAATAAATCCAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 26 (SEQ ID NO: 831) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGCAGCAAAATAAATCCAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 27 (SEQ ID NO: 832) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGAAGCAAAATAAATCCAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 28 (SEQ ID NO: 833) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGGAGCAAAATAAATCCAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 29 (SEQ ID NO: 834) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGATAGCAAAATAAATACTGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 30 (SEQ ID NO: 835) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGATAGCAAAATAAATACCGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 31 (SEQ ID NO: 836) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGATAGCAAAATAAATACAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 32 (SEQ ID NO: 837) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGATAGCAAAATAAATACGGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 33 (SEQ ID NO: 838) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGTAGCAAAATAAATACTGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 34 (SEQ ID NO: 839) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGCAGCAAAATAAATACTGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 35 (SEQ ID NO: 840) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGAAGCAAAATAAATACTGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 36 (SEQ ID NO: 841) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGGAGCAAAATAAATACTGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 37 (SEQ ID NO: 842) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGTAGCAAAATAAATACCGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 38 (SEQ ID NO: 843) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGCAGCAAAATAAATACCGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 39 (SEQ ID NO: 844) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGAAGCAAAATAAATACCGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 40 (SEQ ID NO: 845) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGGAGCAAAATAAATACCGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 41 (SEQ ID NO: 846) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGTAGCAAAATAAATACAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 42 (SEQ ID NO: 847) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGCAGCAAAATAAATACAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 43 (SEQ ID NO: 848) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGAAGCAAAATAAATACAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 44 (SEQ ID NO: 849) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGGAGCAAAATAAATACAGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 45 (SEQ ID NO: 850) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGTAGCAAAATAAATACGGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 46 (SEQ ID NO: 851) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGCAGCAAAATAAATACGGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 47 (SEQ ID NO: 852) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGAAGCAAAATAAATACGGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 48 (SEQ ID NO: 853) | GGTCAACAATTGTCGAGAACCTTAGGACATATAATCAGCCTGCG |
| | TTTGTCTACATTCCTATGGCTGGAGAGCTTCGTGGAGGAGCTTGG |
| | GTTGTGGTCGGGAGCAAAATAAATACGGACCGCATTGAGTGTTA |
| | TGCTGAGAGGACTGCCAAAGGTAATGTTCTCGAACCTCAAGGGT |
| | TAATTGAAATCAAGTTCAGGTCAGA |
| 49 (SEQ ID NO: 854) | TTGGCAGTCCTCTCAGCATAACACTCAATGCGGTCTGGATTTATT |
| | TTGCTACCGACCACAACCCAAGCTCCTCCACGAAGCTCTCCAGC |
| | CATAGGAATGTA |
| 50 (SEQ ID NO: 855) | TTGGCAGTCCTCTCAGCATAACACTCAATGCGGTCTGGATTTATT |
| | TTGCTGCCGACCACAACCCAAGCTCCTCCACGAAGCTCTCCAGC |
| | CATAGGAATGTA |
| 51 (SEQ ID NO: 856) | TTGGCAGTCCTCTCAGCATAACACTCAATGCGGTCTGGATTTATT |
| | TTGCTTCCGACCACAACCCAAGCTCCTCCACGAAGCTCTCCAGCC |
| | ATAGGAATGTA |
| 52 (SEQ ID NO: 857) | TTGGCAGTCCTCTCAGCATAACACTCAATGCGGTCTGGATTTATT |
| | TTGCTCCCGACCACAACCCAAGCTCCTCCACGAAGCTCTCCAGCC |
| | ATAGGAATGTA |
| 53 (SEQ ID NO: 858) | TTGGCAGTCCTCTCAGCATAACACTCAATGCGGTCAGTATTTATT |
| | TTGCTATCGACCACAACCCAAGCTCCTCCACGAAGCTCTCCAGCC |
| | ATAGGAATGTA |
| 54 (SEQ ID NO: 859) | TTGGCAGTCCTCTCAGCATAACACTCAATGCGGTCGGTATTTATT |
| | TTGCTATCGACCACAACCCAAGCTCCTCCACGAAGCTCTCCAGCC |
| | ATAGGAATGTA |
| 55 (SEQ ID NO: 860) | TTGGCAGTCCTCTCAGCATAACACTCAATGCGGTCTGTATTTATT |
| | TTGCTATCGACCACAACCCAAGCTCCTCCACGAAGCTCTCCAGCC |
| | ATAGGAATGTA |
| 56 (SEQ ID NO: 861) | TTGGCAGTCCTCTCAGCATAACACTCAATGCGGTCCGTATTTATT |
| | TTGCTATCGACCACAACCCAAGCTCCTCCACGAAGCTCTCCAGCC |
| | ATAGGAATGTA |
| 57 (SEQ ID NO: 862) | TTGGCAGTCCTCTCAGCATAACACTCAATGCGGTCAGTATTTATT |
| | TTGCTACCGACCACAACCCAAGCTCCTCCACGAAGCTCTCCAGC |
| | CATAGGAATGTA |
| 58 (SEQ ID NO: 863) | TTGGCAGTCCTCTCAGCATAACACTCAATGCGGTCAGTATTTATT |
| | TTGCTGCCGACCACAACCCAAGCTCCTCCACGAAGCTCTCCAGC |
| | CATAGGAATGTA |
| 59 (SEQ ID NO: 864) | TTGGCAGTCCTCTCAGCATAACACTCAATGCGGTCAGTATTTATT |
| | TTGCTTCCGACCACAACCCAAGCTCCTCCACGAAGCTCTCCAGCC |
| | ATAGGAATGTA |
| 60 (SEQ ID NO: 865) | TTGGCAGTCCTCTCAGCATAACACTCAATGCGGTCAGTATTTATT |
| | TTGCTCCCGACCACAACCCAAGCTCCTCCACGAAGCTCTCCAGCC |
| | ATAGGAATGTA |
| 61 (SEQ ID NO: 866) | TTGGCAGTCCTCTCAGCATAACACTCAATGCGGTCGGTATTTATT |
| | TTGCTACCGACCACAACCCAAGCTCCTCCACGAAGCTCTCCAGC |
| | CATAGGAATGTA |
| 62 (SEQ ID NO: 867) | TTGGCAGTCCTCTCAGCATAACACTCAATGCGGTCGGTATTTATT |
| | TTGCTGCCGACCACAACCCAAGCTCCTCCACGAAGCTCTCCAGC |
| | CATAGGAATGTA |
| 63 (SEQ ID NO: 868) | TTGGCAGTCCTCTCAGCATAACACTCAATGCGGTCGGTATTTATT |
| | TTGCTTCCGACCACAACCCAAGCTCCTCCACGAAGCTCTCCAGCC |
| | ATAGGAATGTA |
| 64 (SEQ ID NO: 869) | TTGGCAGTCCTCTCAGCATAACACTCAATGCGGTCGGTATTTATT |
| | TTGCTCCCGACCACAACCCAAGCTCCTCCACGAAGCTCTCCAGCC |
| | ATAGGAATGTA |
| 65 (SEQ ID NO: 870) | TTGGCAGTCCTCTCAGCATAACACTCAATGCGGTCTGTATTTATT |
| | TTGCTACCGACCACAACCCAAGCTCCTCCACGAAGCTCTCCAGC |
| | CATAGGAATGTA |
| 66 (SEQ ID NO: 871) | TTGGCAGTCCTCTCAGCATAACACTCAATGCGGTCTGTATTTATT |
| | TTGCTGCCGACCACAACCCAAGCTCCTCCACGAAGCTCTCCAGC |
| | CATAGGAATGTA |
| 67 (SEQ ID NO: 872) | TTGGCAGTCCTCTCAGCATAACACTCAATGCGGTCTGTATTTATT |
| | TTGCTTCCGACCACAACCCAAGCTCCTCCACGAAGCTCTCCAGCC |
| | ATAGGAATGTA |
| 68 (SEQ ID NO: 873) | TTGGCAGTCCTCTCAGCATAACACTCAATGCGGTCTGTATTTATT |
| | TTGCTCCCGACCACAACCCAAGCTCCTCCACGAAGCTCTCCAGCC |
| | ATAGGAATGTA |
| 69 (SEQ ID NO: 874) | TTGGCAGTCCTCTCAGCATAACACTCAATGCGGTCCGTATTTATT |
| | TTGCTACCGACCACAACCCAAGCTCCTCCACGAAGCTCTCCAGC |
| | CATAGGAATGTA |
| 70 (SEQ ID NO: 875) | TTGGCAGTCCTCTCAGCATAACACTCAATGCGGTCCGTATTTATT |
| | TTGCTGCCGACCACAACCCAAGCTCCTCCACGAAGCTCTCCAGC |
| | CATAGGAATGTA |
| 71 (SEQ ID NO: 876) | TTGGCAGTCCTCTCAGCATAACACTCAATGCGGTCCGTATTTATT |
| | TTGCTTCCGACCACAACCCAAGCTCCTCCACGAAGCTCTCCAGCC |
| | ATAGGAATGTA |
| 72 (SEQ ID NO: 877) | TTGGCAGTCCTCTCAGCATAACACTCAATGCGGTCCGTATTTATT |
| | TTGCTCCCGACCACAACCCAAGCTCCTCCACGAAGCTCTCCAGCC |
| | ATAGGAATGTA |
| 73 (SEQ ID NO: 878) | TCTGACCTGAACTTGATTTCAATTAACCCTTGAGGTTCGAGAACA |
| | TTACCTTTGGCAGTCCTCTCAGCATAACACTCAATGCGGTCTGGA |
| | TTTATTTTGCTACCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 74 (SEQ ID NO: 879) | TCTGACCTGAACTTGATTTCAATTAACCCTTGAGGTTCGAGAACA |
| | TTACCTTTGGCAGTCCTCTCAGCATAACACTCAATGCGGTCTGGA |
| | TTTATTTTGCTGCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 75 (SEQ ID NO: 880) | TCTGACCTGAACTTGATTTCAATTAACCCTTGAGGTTCGAGAACA |
| | TTACCTTTGGCAGTCCTCTCAGCATAACACTCAATGCGGTCTGGA |
| | TTTATTTTGCTTCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 76 (SEQ ID NO: 881) | TCTGACCTGAACTTGATTTCAATTAACCCTTGAGGTTCGAGAACA |
| | TTACCTTTGGCAGTCCTCTCAGCATAACACTCAATGCGGTCTGGA |
| | TTTATTTTGCTCCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 77 (SEQ ID NO: 882) | TCTGACCTGAACTTGATTTCAATTAACCCTTGAGGTTCGAGAACA |
| | TTACCTTTGGCAGTCCTCTCAGCATAACACTCAATGCGGTCAGTA |
| | TTTATTTTGCTATCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 78 (SEQ ID NO: 883) | TCTGACCTGAACTTGATTTCAATTAACCCTTGAGGTTCGAGAACA |
| | TTACCTTTGGCAGTCCTCTCAGCATAACACTCAATGCGGTCGGTA |
| | TTTATTTTGCTATCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 79 (SEQ ID NO: 884) | TCTGACCTGAACTTGATTTCAATTAACCCTTGAGGTTCGAGAACA |
| | TTACCTTTGGCAGTCCTCTCAGCATAACACTCAATGCGGTCTGTA |
| | TTTATTTTGCTATCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 80 (SEQ ID NO: 885) | TCTGACCTGAACTTGATTTCAATTAACCCTTGAGGTTCGAGAACA |
| | TTACCTTTGGCAGTCCTCTCAGCATAACACTCAATGCGGTCCGTA |
| | TTTATTTTGCTATCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 81 (SEQ ID NO: 886) | TCTGACCTGAACTTGATTTCAATTAACCCTTGAGGTTCGAGAACA |
| | TTACCTTTGGCAGTCCTCTCAGCATAACACTCAATGCGGTCAGTA |
| | TTTATTTTGCTACCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 82 (SEQ ID NO: 887) | TCTGACCTGAACTTGATTTCAATTAACCCTTGAGGTTCGAGAACA |
| | TTACCTTTGGCAGTCCTCTCAGCATAACACTCAATGCGGTCAGTA |
| | TTTATTTTGCTGCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 83 (SEQ ID NO: 888) | TCTGACCTGAACTTGATTTCAATTAACCCTTGAGGTTCGAGAACA |
| | TTACCTTTGGCAGTCCTCTCAGCATAACACTCAATGCGGTCAGTA |
| | TTTATTTTGCTTCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 84 (SEQ ID NO: 889) | TCTGACCTGAACTTGATTTCAATTAACCCTTGAGGTTCGAGAACA |
| | TTACCTTTGGCAGTCCTCTCAGCATAACACTCAATGCGGTCAGTA |
| | TTTATTTTGCTCCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 85 (SEQ ID NO: 890) | TCTGACCTGAACTTGATTTCAATTAACCCTTGAGGTTCGAGAACA |
| | TTACCTTTGGCAGTCCTCTCAGCATAACACTCAATGCGGTCGGTA |
| | TTTATTTTGCTACCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 86 (SEQ ID NO: 891) | TCTGACCTGAACTTGATTTCAATTAACCCTTGAGGTTCGAGAACA |
| | TTACCTTTGGCAGTCCTCTCAGCATAACACTCAATGCGGTCGGTA |
| | TTTATTTTGCTGCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 87 (SEQ ID NO: 892) | TCTGACCTGAACTTGATTTCAATTAACCCTTGAGGTTCGAGAACA |
| | TTACCTTTGGCAGTCCTCTCAGCATAACACTCAATGCGGTCGGTA |
| | TTTATTTTGCTTCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 88 (SEQ ID NO: 893) | TCTGACCTGAACTTGATTTCAATTAACCCTTGAGGTTCGAGAACA |
| | TTACCTTTGGCAGTCCTCTCAGCATAACACTCAATGCGGTCGGTA |
| | TTTATTTTGCTCCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 89 (SEQ ID NO: 894) | TCTGACCTGAACTTGATTTCAATTAACCCTTGAGGTTCGAGAACA |
| | TTACCTTTGGCAGTCCTCTCAGCATAACACTCAATGCGGTCTGTA |
| | TTTATTTTGCTACCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 90 (SEQ ID NO: 895) | TCTGACCTGAACTTGATTTCAATTAACCCTTGAGGTTCGAGAACA |
| | TTACCTTTGGCAGTCCTCTCAGCATAACACTCAATGCGGTCTGTA |
| | TTTATTTTGCTGCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 91 (SEQ ID NO: 896) | TCTGACCTGAACTTGATTTCAATTAACCCTTGAGGTTCGAGAACA |
| | TTACCTTTGGCAGTCCTCTCAGCATAACACTCAATGCGGTCTGTA |
| | TTTATTTTGCTTCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 92(SEQ ID NO: 897) | TCTGACCTGAACTTGATTTCAATTAACCCTTGAGGTTCGAGAACA |
| | TTACCTTTGGCAGTCCTCTCAGCATAACACTCAATGCGGTCTGTA |
| | TTTATTTTGCTCCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 93 (SEQ ID NO: 898) | TCTGACCTGAACTTGATTTCAATTAACCCTTGAGGTTCGAGAACA |
| | TTACCTTTGGCAGTCCTCTCAGCATAACACTCAATGCGGTCCGTA |
| | TTTATTTTGCTACCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 94 (SEQ ID NO: 899) | TCTGACCTGAACTTGATTTCAATTAACCCTTGAGGTTCGAGAACA |
| | TTACCTTTGGCAGTCCTCTCAGCATAACACTCAATGCGGTCCGTA |
| | TTTATTTTGCTGCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 95 (SEQ ID NO: 900) | TCTGACCTGAACTTGATTTCAATTAACCCTTGAGGTTCGAGAACA |
| | TTACCTTTGGCAGTCCTCTCAGCATAACACTCAATGCGGTCCGTA |
| | TTTATTTTGCTTCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 96 (SEQ ID NO: 901) | TCTGACCTGAACTTGATTTCAATTAACCCTTGAGGTTCGAGAACA |
| | TTACCTTTGGCAGTCCTCTCAGCATAACACTCAATGCGGTCCGTA |
| | TTTATTTTGCTCCCGACCACAACCCAAGCTCCTCCACGAAGCTCT |
| | CCAGCCATAGGAATGTAGACAAACGCAGGCTGATTATATGTCCT |
| | AAGGTTCTCGACAATTGTTGACC |
| 97 (SEQ ID NO: 902) | TACATTCCTATGGCTGGAGAGCTTCGTGGAGGAGCTTGGGTTGT |
| | GGTCGGNAGCAAAATAAATACNGACCGCATTGAGTGTTATGCTG |
| | AGAGGACTGCCAA |
| 98 (SEQ ID NO: 903) | TTGGCAGTCCTCTCAGCATAACACTCAATGCGGTCNGTATTTATT |
| | TTGCTNCCGACCACAACCCAAGCTCCTCCACGAAGCTCTCCAGC |
| | CATAGGAATGTA |

### Example 15

A method to introduce targeted mutations into a native barley Hordeum vulgare ACCase gene utilizes PEG delivery of CRISPR-Cas9/Cas12a and GRONs into protoplasts. Examples of target spacer sequences for sgRNA targeting the barley ACCase gene are shown, but not limited to, those listed in the below Table 20. Sequence information for exemplary GRONs is shown, but not limited to those listed in the below Table 21.

TABLE 20
Exemplary target spacer sequences for barley ACCase sgRNA

| Sequence No. | 5′ to 3′ sequence |
|---|---|
| 1 (SEQ ID NO: 904) | ggagctacgtggaggggctt |
| 2 (SEQ ID NO: 905) | cagagctacgtggaggggct |
| 3 (SEQ ID NO: 906) | agagctacgtggaggggctt |

TABLE 21
Exemplary barley ACCase GRON Sequences

| Sequence No. | 5′ to 3′ (V = CY3; H = 3′DMT dC CPG) |
|---|---|
| 1 (SEQ ID NO: 907) | TATATCCCCAAGGCTGCAGAGCTACGTGGAGGGGCTTGGGTTGT |
| | GATTGGTAGCAAGATAAATACAGATCGCATTGAGTTCTATGCTG |
| | AGAGGACTGCAAA |
| 2 (SEQ ID NO: 908) | TTTGCAGTCCTCTCAGCATAGAACTCAATGCGATCTGTATTTATC |
| | TTGCTACCAATCACAACCCAAGCCCCTCCACGTAGCTCTGCAGCC |
| | TTGGGGATATA |
| 3 (SEQ ID NO: 909) | TATATCCCCAAGGCTGCAGAGCTACGTGGAGGGGCTTGGGTTGT |
| | GATTGGNAGCAAGATAAATACNGATCGCATTGAGTTCTATGCTG |
| | AGAGGACTGCAAA |
| 4 (SEQ ID NO: 910) | TTTGCAGTCCTCTCAGCATAGAACTCAATGCGATCNGTATTTATC |
| | TTGCTNCCAATCACAACCCAAGCCCCTCCACGTAGCTCTGCAGCC |
| | TTGGGGATATA |

### Example 16

A method to introduce targeted mutations into a native Kentucky blue-grass Poa pratensis ACCase gene utilizes PEG delivery of CRISPR-Cas9/Cas12a and GRONs into protoplasts. Examples of target spacer sequences for sgRNA targeting the Kentucky blue-grass ACCase gene are shown, but not limited to, those listed in the below Table 22. Sequence information for exemplary GRONs is shown, but not limited to those listed in the below Table 23.

TABLE 22
Exemplary target spacer sequences for Kentucky
blue-grass ACCase sgRNA

| Sequence No. | 5′ to 3′ sequence |
|---|---|
| 1 (SEQ ID NO: 911) | ggagctacgtggaggggctt |
| 2 (SEQ ID NO: 912) | cagagctgcgtggaggggct |
| 3 (SEQ ID NO: 913) | agagctgcgtggaggggctt |

TABLE 23
Exemplary Kentucky blue-grass ACCase GRON Sequences

| Sequence No. | 5′ to 3′ (V = CY3; H = 3′DMT dC CPG) |
|---|---|
| 1 (SEQ ID NO: 914) | TATATCCCCAAGGCTGCAGAGCTACGTGGAGGGGCTTGGGTCGT |
| | GATTGGTAGCAAGATAAACACAGATCGGATTGAGTGCTACGCTG |
| | AGAGGACCGCAAA |
| 2 (SEQ ID NO: 915) | TTTGCGGTCCTCTCAGCGTAGCACTCAATCCGATCTGTGTTTATC |
| | TTGCTACCAATCACGACCCAAGCCCCTCCACGTAGCTCTGCAGCC |
| | TTGGGGATATA |
| 3 (SEQ ID NO: 916) | TATATCCCCAAGGCTGCAGAGCTACGTGGAGGGGCTTGGGTCGT |
| | GATTGGTAGCAAGATAAACACAGATCGCATTGAGTGCTATGCTG |
| | AGAGGACTGCAAA |
| 4 (SEQ ID NO: 917) | TTTGCAGTCCTCTCAGCATAGCACTCAATGCGATCTGTGTTTATC |
| | TTGCTACCAATCACGACCCAAGCCCCTCCACGTAGCTCTGCAGCC |
| | TTGGGGATATA |
| 5 (SEQ ID NO: 918) | TATATCCCCAAGGCTGCAGAGCTACGTGGAGGGGCTTGGGTCGT |
| | GATTGGNAGCAAGATAAACACNGATCGGATTGAGTGCTACGCTG |
| | AGAGGACCGCAAA |
| 6 (SEQ ID NO: 919) | TTTGCGGTCCTCTCAGCGTAGCACTCAATCCGATCNGTGTTTATC |
| | TTGCTNCCAATCACGACCCAAGCCCCTCCACGTAGCTCTGCAGCC |
| | TTGGGGATATA |
| 7 (SEQ ID NO: 920) | TATATCCCCAAGGCTGCAGAGCTACGTGGAGGGGCTTGGGTCGT |
| | GATTGGNAGCAAGATAAACACNGATCGCATTGAGTGCTATGCTG |
| | AGAGGACTGCAAA |
| 8 (SEQ ID NO: 921) | TTTGCAGTCCTCTCAGCATAGCACTCAATGCGATCNGTGTTTATC |
| | TTGCTNCCAATCACGACCCAAGCCCCTCCACGTAGCTCTGCAGCC |
| | TTGGGGATATA |

### Example 17

A method to introduce targeted mutations into a native oats Avena sativa ACCase gene utilizes PEG delivery of CRISPR-Cas9/Cas12a and GRONs into protoplasts. Examples of target spacer sequences for sgRNA targeting the oats ACCase gene are shown, but not limited to, those listed in the below Table 24. Sequence information for exemplary GRONs is shown, but not limited to those listed in the below Table 25.

TABLE 24
Exemplary target spacer sequences for oats
ACCase sgRNA

| Sequence No. | 5′ to 3′ sequence |
|---|---|
| 1 (SEQ ID NO: 922) | ggagctgcgtggaggggctt |
| 2 (SEQ ID NO: 923) | cagagctacgtggaggggct |
| 3 (SEQ ID NO: 924) | agagctacgtggaggggctt |

TABLE 25
Exemplary oats ACCase GRON Sequences

| Sequence No. | 5′ to 3′ (V = CY3; H = 3′DMT dC CPG) |
|---|---|
| 1 (SEQ ID NO: 925) | TATATCCCCAAGGCTGCAGAGCTGCGTGGAGGGGCTTGGGTCGT |
| | GATTGGTAGCAAGATAAATACAGATCGCATTGAGTGCTATGCTG |
| | AGACGACTGCAAA |
| 2 (SEQ ID NO: 926) | TTTGCAGTCGTCTCAGCATAGCACTCAATGCGATCTGTATTTATC |
| | TTGCTACCAATCACGACCCAAGCCCCTCCACGCAGCTCTGCAGC |
| | CTTGGGGATATA |
| 3 (SEQ ID NO: 927) | TATATCCCCAAGGCTGCAGAGCTGCGTGGAGGGGCTTGGGTCGT |
| | GATTGGNAGCAAGATAAATACNGATCGCATTGAGTGCTATGCTG |
| | AGACGACTGCAAA |
| 4 (SEQ ID NO: 928) | TTTGCAGTCGTCTCAGCATAGCACTCAATGCGATCNGTATTTATC |
| | TTGCTNCCAATCACGACCCAAGCCCCTCCACGCAGCTCTGCAGC |
| | CTTGGGGATATA |

### Example 18

A method to introduce targeted mutations into a native foxtail millet Setaria italica ACCase gene utilizes PEG delivery of CRISPR-Cas9/Cas12a and GRONs into protoplasts. Examples of target spacer sequences for sgRNA targeting the foxtail millet ACCase gene are shown, but not limited to, those listed in the below Table 26. Sequence information for exemplary GRONs is shown, but not limited to those listed in the below Table 27.

TABLE 26
Exemplary target spacer sequences for foxtail
millet ACCase sgRNA

| Sequence No. | 5′ to 3′ sequence |
|---|---|
| 1 (SEQ ID NO: 929) | ggagctgcgtggaggagctt |
| 2 (SEQ ID NO: 930) | agagctgcgtggaggagctt |
| 3 (SEQ ID NO: 931) | gagagctgcgtggaggagct |

TABLE 27
Exemplary foxtail millet ACCase GRON Sequences

| Sequence No. | 5′ to 3′ (V = CY3; H = 3′DMT dC CPG) |
|---|---|
| 1 (SEQ ID NO: 932) | TACATTCCTATGGCTGGAGAGCTGCGTGGAGGAGCTTGGGTTGT |
| | GGTTGGTAGCAAAATAAATACAGACCGAATTGAGTGTTATGCTG |
| | AGAGGACTGCTAA |
| 2 (SEQ ID NO: 933) | TTAGCAGTCCTCTCAGCATAACACTCAATTCGGTCTGTATTTATT |
| | TTGCTACCAACCACAACCCAAGCTCCTCCACGCAGCTCTCCAGCC |
| | ATAGGAATGTA |
| 3 (SEQ ID NO: 934) | TACATTCCTATGGCTGGAGAGCTGCGTGGAGGAGCTTGGGTTGT |
| | GGTTGGNAGCAAAATAAATACNGACCGAATTGAGTGTTATGCTG |
| | AGAGGACTGCTAA |
| 4 (SEQ ID NO: 935) | TTAGCAGTCCTCTCAGCATAACACTCAATTCGGTCNGTATTTATT |
| | TTGCTNCCAACCACAACCCAAGCTCCTCCACGCAGCTCTCCAGCC |
| | ATAGGAATGTA |

### Example 19

A method to introduce targeted mutations into a native switchgrass Panicum virgatum ACCase gene utilizes PEG delivery of CRISPR-Cas9/Cas12a and GRONs into protoplasts. Examples of target spacer sequences for sgRNA targeting the switchgrass ACCase gene are shown, but not limited to, those listed in the below Table 28. Sequence information for exemplary GRONs is shown, but not limited to those listed in the below Table 29.

TABLE 28
Exemplary target spacer sequences for switchgrass
ACCase sgRNA

| Sequence No. | 5′ to 3′ sequence |
|---|---|
| 1 (SEQ ID NO: 936) | ggagctacgtggtggagctt |
| 2 (SEQ ID NO: 937) | gagagctacgtggtggagct |
| 3 (SEQ ID NO: 938) | agagctacgtggtggagctt |

TABLE 29
Exemplary switchgrass ACCase GRON Sequences

| Sequence No. | 5′ to 3′ (V = CY3; H = 3′DMT dC CPG) |
|---|---|
| 1 (SEQ ID NO: 939) | TACATTCCTATGGCTGGAGAGCTACGTGGTGGAGCTTGGGTTGTT |
| | GTTGGTAGCAAAATAAATACAGACCGAATTGAGTGTTATGCTGA |
| | GAGGACTGCGAA |
| 2 (SEQ ID NO: 940) | TTCGCAGTCCTCTCAGCATAACACTCAATTCGGTCTGTATTTATT |
| | TTGCTACCAACAACAACCCAAGCTCCACCACGTAGCTCTCCAGC |
| | CATAGGAATGTA |
| 3 (SEQ ID NO: 941) | TACATTCCTATGGCTGGAGAGCTACGTGGTGGAGCTTGGGTTGTT |
| | GTTGGNAGCAAAATAAATACNGACCGAATTGAGTGTTATGCTGA |
| | GAGGACTGCGAA |
| 4 (SEQ ID NO: 942) | TTCGCAGTCCTCTCAGCATAACACTCAATTCGGTCNGTATTTATT |
| | TTGCTNCCAACAACAACCCAAGCTCCACCACGTAGCTCTCCAGC |
| | CATAGGAATGTA |

### Example 20

A method to introduce targeted mutations into a native perennial ryegrass Lolium perenne ACCase gene utilizes PEG delivery of CRISPR-Cas9/Cas12a and GRONs into protoplasts. Examples of target spacer sequences for sgRNA targeting the perennial ryegrass ACCase gene are shown, but not limited to, those listed in the below Table 30. Sequence information for exemplary GRONs is shown, but not limited to those listed in the below Table 31.

TABLE 30
Exemplary target spacer sequences for perennial
kengrass ACCase sgRNA

| Sequence No. | 5′ to 3′ sequence |
|---|---|
| 1 (SEQ ID NO: 943) | ggagctacgtggaggtgctt |
| 2 (SEQ ID NO: 944) | agagctacgtggaggtgctt |
| 3 (SEQ ID NO: 945) | cagagctacgtggaggtgct |

TABLE 31
Exemplary perennial ryegrass ACCase GRON Sequences

| Sequence No. | 5′ to 3′ (V = CY3; H = 3′DMT dC CPG) |
|---|---|
| 1 (SEQ ID NO: 946) | TATATCCCCAAGGCTGCAGAGCTACGTGGAGGTGCTTGGGTCGT |
| | GATTGGTAGCAAGATAAATACAGATCGCATTGAGTGCTATGCTG |
| | AGACAACTGCAAA |
| 2 (SEQ ID NO: 947) | TTTGCAGTTGTCTCAGCATAGCACTCAATGCGATCTGTATTTATC |
| | TTGCTACCAATCACGACCCAAGCACCTCCACGTAGCTCTGCAGC |
| | CTTGGGGATATA |
| 3 (SEQ ID NO: 948) | TATATCCCCAAGGCTGCAGAGCTACGTGGAGGTGCTTGGGTCGT |
| | GATTGGNAGCAAGATAAATACNGATCGCATTGAGTGCTATGCTG |
| | AGACAACTGCAAA |
| 4 (SEQ ID NO: 949) | TTTGCAGTTGTCTCAGCATAGCACTCAATGCGATCNGTATTTATC |
| | TTGCTNCCAATCACGACCCAAGCACCTCCACGTAGCTCTGCAGC |
| | CTTGGGGATATA |

One skilled in the art readily appreciates that the present disclosure is well adapted to carry out the objects and obtain the ends and advantages mentioned, as well as those inherent therein. The examples provided herein are representative of preferred embodiments, are exemplary, and are not intended as limitations on the scope of the disclosure.

It will be readily apparent to a person skilled in the art that varying substitutions and modifications may be made to the disclosure disclosed herein without departing from the scope and spirit of the disclosure.

The disclosure illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

Thus, it should be understood that although the present disclosure has been specifically disclosed by preferred embodiments and optional features, modification, improvement, and variation of the disclosures disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

We claim:

1. A plant or plant cell having two or more mutations in an ACCase gene such that the mutated gene encodes an ACCase protein comprising a first amino acid substitution at a position corresponding to position D2078 of SEQ ID NO: 2 and a second amino acid substitution at a position corresponding to position P2083 of SEQ ID NO: 2.

2. The plant or plant cell of claim 1, wherein the first substitution is selected from D2078G, D2078K, D2078T, and D2078V.

3. The plant or plant cell of claim 1 or 2, wherein the first substitution is D2078G.

4. The plant or plant cell of any of the preceding claims, wherein the second substitution is selected from P2083T, P2083S, P2083N, and P2083Q.

5. The plant or plant cell of any of the preceding claims, wherein the second substitution is P2083T.

6. The plant or plant cell of any of the preceding claims, wherein the first substitution is D2078G, and the second substitution is P2083T.

7. The plant or plant cell of any of the preceding claims, wherein the plant or plant cell belongs to the genus Oryza.

8. An Oryza plant or plant cell having two or more mutations in an ACCase gene such that the mutated gene encodes a protein comprising a first amino acid substitution at a position corresponding to position D2089 of SEQ ID NO: 4 and a second amino acid substitution at a position corresponding to position P2094 of SEQ ID NO: 4; or a first amino acid substitution at a position corresponding to position D2089 of SEQ ID NO: 954 and a second amino acid substitution at a position corresponding to position P2094 of SEQ ID NO: 954.

9. The Oryza plant or plant cell of claim 8, wherein the first substitution is D2089G.

10. The Oryza plant or plant cell of any of the preceding claims, wherein the second substitution is P2094T.

11. The Oryza plant or plant cell of any of the preceding claims, wherein the first substitution is D2089G, and the second substitution is P2094T.

12. An Oryza plant or plant cell having two or more mutations in an ACCase gene such that the mutated gene encodes a protein comprising a first amino acid substitution at a position corresponding to position D2076 of SEQ ID NO: 6 and a second amino acid substitution at a position corresponding to position P2081 of SEQ ID NO: 6.

13. The Oryza plant or plant cell of claim 12, wherein the first substitution D2076G.

14. The Oryza plant or plant cell of any of the preceding claims, wherein the second substitution is P2081T.

15. The Oryza plant or plant cell of any of the preceding claims, wherein the first substitution is D2076G, and the second substitution is P2081T.

16. A plant or plant cell expressing an ACCase protein comprising a first amino acid substitution at a position corresponding to position D2078 of SEQ ID NO: 2 and a second amino acid substitution at a position corresponding to position P2083 of SEQ ID NO: 2.

17. The plant or plant cell of claim 16, wherein the first substitution is selected from D2078G, D2078K, D2078T, and D2078V.

18. The plant or plant cell of claim 16 or 17, wherein the first substitution is D2078G.

19. The plant or plant cell of one of claims 16-18, wherein the second substitution is selected from P2083T, P2083S, P2083N, and P2083Q.

20. The plant or plant cell of one of claims 16-19, wherein the second substitution is P2083T.

21. The plant or plant cell of one of claims 16-20, wherein the first substitution is D2078G, and the second substitution is P2083T.

22. The plant or plant cell of one of claims 16-21, wherein the plant or plant cell belongs to the genus Oryza.

23. An Oryza plant or plant cell expressing a protein comprising a first amino acid substitution at a position corresponding to position D2089 of SEQ ID NO: 4 and a second amino acid substitution at a position corresponding to position P2094 of SEQ ID NO: 4; or a first amino acid substitution at a position corresponding to position D2089 of SEQ ID NO: 954 and a second amino acid substitution at a position corresponding to position P2094 of SEQ ID NO: 954.

24. The Oryza plant or plant cell of claim 23, wherein the first substitution is D2089G.

25. The Oryza plant or plant cell of one of claim 23 or 24, wherein the second substitution is P2094T.

26. The Oryza plant or plant cell of one of claims 23-25, wherein the first substitution is D2089G, and the second substitution is P2094T.

27. An Oryza plant or plant cell expressing a protein comprising a first amino acid substitution at a position corresponding to position D2076 of SEQ ID NO: 6 and a second amino acid substitution at a position corresponding to position P2081 of SEQ ID NO: 6.

28. The Oryza plant or plant cell of claim 27, wherein the first substitution D2076G.

29. The Oryza plant or plant cell of one of claim 27 or 28, wherein the second substitution is P2081T.

30. The Oryza plant or plant cell of one of claims 27-29, wherein the first substitution is D2076G, and the second substitution is P2081T.

31. The plant or plant cell of any of the preceding claims wherein such plant or plant cell is resistant to one or more of alloxydim, butroxydim, clethodim, cloproxydim, cycloxydim, profoxydim, sethoxydim, tepraloxydim, tralkoxydim, chlorazifop, clodinafop, clofop, diclofop-methyl, fenoxaprop, fenoxaprop-P-ethyl, fenthiaprop, fluazifop, fluazifop-P-butyl, haloxyfop, haloxyfop-P-methyl, isoxapyrifop, propaquizafop, quizalofop, quizalofop-P-ethyl, trifop, pinoxaden, or an agronomically acceptable salts or esters thereof.

32. The plant or plant cell of any of the preceding claims wherein such plant or plant cell is resistant to clethodim.

33. The plant or plant cell of any of the preceding claims wherein such plant or plant cell is fertile.

34. The plant or plant cell of any of the preceding claims wherein such plant or plant cell nontransgenic with respect to the ACCase protein.

35. The plant or plant cell of any of the preceding claims wherein the plant species is selected from the group consisting of canola, sunflower, corn, tobacco, sugar beet, cotton, wheat (including but not limited to Triticum spp., Triticum aestivum, Triticum durum Triticum timopheevii, Triticum monococcum, Triticum spelta, Triticum zhukovskyi and Triticum urartu and hybrids thereof), barley (including but not limited to Hordeum vulgare L., Hordeum comosum, Hordeum depressum, Hordeum intercedens, Hordeum jubatum, Hordeum marinum, Hordeum marinum, Hordeum parodii, Hordeum pusillum, Hordeum secalinum, and Hordeum spontaneum), rice (including but not limited to Oryza sativa subsp. indica, Oryza sativa subsp. japonica, Oryza sativa subsp. javanica, Oryza sativa subsp. glutinosa (glutinous rice), Oryza sativa Aromatica group (e.g., basmati), and Oryza sativa (floating rice group)), alfalfa, sorghum (e.g., Sorghum bicolor), foxtail millet (e.g., Setaria italica), switchgrass (e.g., Panicum virgatum), Kentucky blue-grass (e.g., Poa pratensis), tomato, mango, peach, apple, pear, strawberry, banana, melon, cassava, potato, carrot, lettuce, onion, soy bean, soya spp, sugar cane, pea, chickpea, field pea, fava bean, lentils, turnip, rutabaga, brussels sprouts, lupin, cauliflower, kale, field beans, poplar, pine, eucalyptus, grape, citrus, triticale, alfalfa, rye (including but not limited to Secale sylvestre, Secale strictum, Secale cereale, Secale vavilovii, Secale africanum, Secale ciliatoglume, Secale ancestrale, and Secale montanum), perennial ryegrass (e.g., Lolium perenne), oats (e.g., Avena sativa), turf (including but not limited to Turf grass include Zoysia japonica, Agrostris palustris, Poa pratensis, Poa annua, Digitaria sanguinalis, Cyperus rotundus, Kyllinga brevifolia, Cyperus amuricus, Erigeron canadensis, Hydrocotyle sibthorpioides, Kummerowia striata, Euphorbia humifusa, and Viola arvensis) and forage grasses, flax, oilseed rape, cotton, mustard, cucumber, morning glory, balsam, pepper, eggplant, marigold, lotus, cabbage, daisy, carnation, tulip, iris, and lily.

36. A method of making a plant or plant cell of any of the preceding claims comprising modifying an ACCase gene of a plant or plant cell.

37. A method of making a plant or plant cell of any of the preceding claims, said method comprising exposing a plant cell to a DNA cutter and a modified GRON configured to introduce one or more base pair mismatches with a genomic target site.

38. A method according to claim 37, wherein the DNA cutter is site-specific and is configured to introduce a double strand break or a single strand nick within 2 kb of the genomic target site.

39. A method for making a plant or plant cell of any of the preceding claims comprising:
a) introducing into plant cells a gene repair oligonucleobase configured to introduce one or more a targeted mutations in the ACCase gene to produce plant cells with a mutant ACCase gene that expresses an ACCase protein that is substituted at one or more amino acid positions,
b) selecting a plant cell exhibiting improved tolerance to one or more of alloxydim, butroxydim, clethodim, cloproxydim, cycloxydim, sethoxydim, tepraloxydim, tralkoxydim, chlorazifop, clodinafop, clofop, diclofop, fenoxaprop, fenoxaprop-P, fenthiaprop, fluazifop, fluazifop-P, haloxyfop, haloxyfop-P, isoxapyrifop, propaquizafop, quizalofop, quizalofop-P, trifop, pinoxaden, agronomically acceptable salts and esters of any of these herbicides, and combinations thereof as compared to a corresponding wild-type plant cell; and
c) regenerating a non-transgenic herbicide resistant plant having a mutated ACCase gene from said selected plant cell.

40. A method for making a plant or plant cell of any of the preceding claims comprising:
a) introducing into plant cells a gene repair oligonucleobase with a targeted mutation in the ACCase gene to produce plant cells with a mutant ACCase gene that expresses an ACCase protein that is substituted at one or more amino acid positions,
b) selecting a plant cell exhibiting improved tolerance to one or more of alloxydim, butroxydim, clethodim, cloproxydim, cycloxydim, sethoxydim, tepraloxydim, tralkoxydim, chlorazifop, clodinafop, clofop, diclofop, fenoxaprop, fenoxaprop-P, fenthiaprop, fluazifop, fluazifop-P, haloxyfop, haloxyfop-P, isoxapyrifop, propaquizafop, quizalofop, quizalofop-P, trifop, pinoxaden, agronomically acceptable salts and esters of any of these herbicides, and combinations thereof as compared to a corresponding wild-type plant cell; and
c) regenerating a non-transgenic herbicide tolerant plant having a mutated ACCase gene from said selected plant cell.

41. A method according to claim 39 or 40, further comprising introducing into the plant cells a DNA cutter, wherein the DNA cutter is site-specific and is configured to introduce a double strand break or a single strand nick within 2 kb of the targeted mutation.

42. A method according to claim 41, further comprising culturing and regeneration methods to obtain a plant that produces seeds, and the production of seeds and additional plants including descendant (progeny) plants that contain the mutated ACCase gene.

43. The method of any of the preceeding embodiments, wherein said plant is fertile.

44. A plant ACCase protein comprising a first amino acid substitution at a position corresponding to position D2078 of SEQ ID NO: 2 and a second amino acid substitution at a position corresponding to position P2083 of SEQ ID NO: 2.

45. The plant ACCase protein of claim 44, wherein the first substitution is selected from D2078G, D2078K, D2078T, and D2078V.

46. The plant ACCase protein of claim 44 or 45, wherein the first substitution is D2078G.

47. The plant ACCase protein of one of claims 44-46, wherein the second substitution is selected from P2083T, P2083S, P2083N, and P2083Q.

48. The plant ACCase protein of one of claims 44-47, wherein the second substitution is P2083T.

49. The plant ACCase protein of one of claims 44-47, wherein the first substitution is D2078G and the second substitution is P2083T.

50. The plant ACCase protein of one of claims 44-49, wherein the plant ACCase protein belongs to the genus Oryza.

51. An Oryza ACCase protein comprising a first amino acid substitution at a position corresponding to position D2089 of SEQ ID NO: 4 and a second amino acid substitution at a position corresponding to position P2094 of SEQ ID NO: 4; or a first amino acid substitution at a position corresponding to position D2089 of SEQ ID NO: 954 and a second amino acid substitution at a position corresponding to position P2094 of SEQ ID NO: 954.

52. The Oryza ACCase protein of claim 48, wherein the first substitution is D2089G.

53. The Oryza ACCase protein of one of claim 48 or 49, wherein the second substitution is P2094T.

54. The Oryza ACCase protein of one of claims 48-50, wherein the first substitution is D2089G, and the second substitution is P2094T.

55. An Oryza ACCase protein expressing a protein comprising a first amino acid substitution at a position corresponding to position D2076 of SEQ ID NO: 6 and a second amino acid substitution at a position corresponding to position P2081 of SEQ ID NO: 6.

56. The Oryza ACCase protein of claim 52, wherein the first substitution D2076G.

57. The Oryza ACCase protein of one of claims 52-53, wherein the second substitution is P2081T.

58. The Oryza ACCase protein of one of claims 52-54, wherein the first substitution is D2076G, and the second substitution is P2081T.
